(19) United States
(12) Patent Application Publication
(10) Pub. No.: US 2026/0193273 A9
(43) Pub. Date: Jul. 9, 2026

(54) PIPERAZINE DERIVATIVES USEFUL IN HIV THERAPY

(71) Applicants: VIIV HEALTHCARE COMPANY, Wilimington, DE (US); THE UNIVERSITY OF NORTH CAROLINA AT CHAPEL HILL, Chapel Hill, NC (US)

(72) Inventors: Christopher J. Aquino; Junghyun Lee Suh; Richard M. Dunham

(21) Appl. No.: 18/869,073

(22) PCT Filed: May 25, 2023
(86) PCT No.: PCT/US2023/067445

§ 371 Date: Nov. 25, 2024

Related U.S. Application Data
(60) Provisional application No. 63/346,362, filed on May 27, 2022.

(51) Int. Cl. C07D 519/00; A61K 31/496; A61K 31/5377; A61K 31/551; A61K 45/06; A61P 31/18

(52) U.S. Cl. CPC C07D 519/00; A61K 31/496; A61K 31/5377; A61K 31/551; A61K 45/06; A61P 31/18

(57) ABSTRACT

The invention relates to a compound of Formula (I) or a pharmaceutically acceptable salt thereof, compositions thereof, and methods of therapeutic treatment using the same.

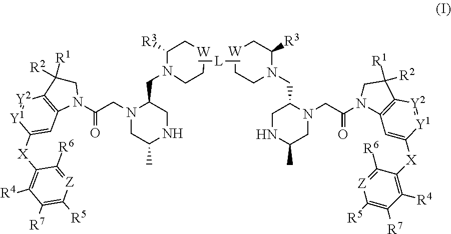

### CROSS REFERENCE TO RELATED APPLICATION

This application claims priority under 35 U.S.C. § 119(e) to U.S. Provisional Application No. 63/346,362, filed on May 27, 2022, the contents of which are incorporated by reference herein in its entirety, including drawings.

### FIELD OF THE INVENTION

The present invention relates to compounds, pharmaceutical compositions, and methods of use thereof in connection with individuals infected with HIV, HBV, or cancer.

### SEQUENCE LISTING

This application contains a Sequence Listing which has been submitted in XML format and is hereby incorporated by reference in its entirety. The XML copy is named PU66990.xml, is about 6.04 KB (6,187 bytes) in size and was created on May 18, 2023.

### BACKGROUND OF THE INVENTION

Human immunodeficiency virus type 1 (HIV-1) infection leads to the contraction of acquired immune deficiency disease (AIDS). Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still believed to be required due to a number of issues including, but not limited to undesirable drug-drug interactions; drug-food interactions; non-adherence to therapy; drug resistance due to mutation of the enzyme target; and inflammation related to the immunologic damage caused by the HIV infection.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur and the survival and quality of life are not normalized as compared to uninfected persons [Lohse Ann Intern Med 2007 146; 87-95]. Indeed, the incidence of several non-AIDS morbidities and mortalities, such as cardiovascular disease, frailty, and neurocognitive impairment, are increased in HAART-suppressed, HIV-infected subjects [Deeks Annu Rev Med 2011; 62:141-155]. This increased incidence of non-AIDS morbidity/mortality occurs in the context of, and is potentially caused by, elevated systemic inflammation related to the immunologic damage caused by HIV infection and residual HIV infection [Hunt J Infect Dis 2014][Byakagwa J Infect Dis 2014][Tenorio J Infect Dis 2014].

Modern antiretroviral therapy (ART) has the ability to effectively suppress HIV replication and improve health outcomes for HIV-infected persons, but is believed to not be capable of completely eliminating HIV viral reservoirs within the individual. HIV genomes can remain latent within mostly immune cells in the infected individual and may reactivate at any time, such that after interruption of ART, virus replication typically resumes within weeks. In a handful of individuals, the size of this viral reservoir has been significantly reduced and upon cessation of ART, the rebound of viral replication has been delayed [Henrich T J J Infect Dis 2013][Henrich T J Ann Intern Med 2014]. In one case, the viral reservoir was eliminated during treatment of leukemia and no viral rebound was observed during several years of follow-up [Hutter G N Engl J Med 2009]. These examples suggest the concept that reduction or elimination of the viral reservoir may be possible and can lead to viral remission or cure. As such, ways have been pursued to eliminate the viral reservoir, by direct molecular means, including excision of viral genomes with CRISPR/Cas9 systems, or to induce reactivation of the latent reservoir during ART so that the latent cells are eliminated. It is believed that reversal of latency is required to make latently infected cells vulnerable to clearance.

SMACm (Second Mitochondrial-derived Activator of Caspases mimetics) are a class of compounds that have recently entered clinical trials as potential cancer treatments. The drugs deplete and/or inhibit cellular inhibitor of apoptosis proteins (cIAP) that act as anti-apoptotic proteins, thereby promoting the cell death of cancer cells. Antagonism and/or depletion of cIAP also leads to activation of the non-canonical NF-kB signaling pathway, that may induce HIV expression and may enable elimination of HIV infected cells. In addition, SMAC mimetics may selectively promote the cell death of cells infected by HIV [Campbell Cell Host Microbe 2018] or HBV [Ebert Proc Nat Acad Sci 2013] by antagonizing anti-apoptotic proteins.

Recently, the targeting of the non-canonical NF-kB (ncNF-κB) pathway to reverse latency in cell line models was reported. The ncNF-κB pathway is typically activated by ligation of a subset of TNF receptor family members. In the steady state, a multimolecular complex with ubiquitin ligase activity consisting of TNF receptor-associated factor 2 (TRAF2), TRAF3, and cellular inhibitor of apoptosis protein-1 (cIAP1) associates with the cytoplasmic portion of the unligated receptor and constitutively ubiquitinylates and degrades the NF-κB-inducing kinase (NIK). Upon receptor ligation, cIAP1 ubiquitinylates TRAF3 and auto-ubiquitinylates, leading to proteasomal degradation of TRAF3 and cIAP1, thereby disinhibiting NIK accumulation. NIK is constitutively active and, once accumulated, phosphorylates the inhibitor of KB kinase-α (IKKα) homodimer. The activated IKKα/IKKα homodimer then phosphorylates the inactive p100 form of NFκB2 leading to ubiquitinylation by Skp1-Cul1-F-box ubiquitin ligase (SCFβTrCP) and proteasomal cleavage of p100, releasing the active p52 subunit. p52 associates with RelB, and this heterodimer translocates into the nucleus to drive transcription from KB promoter elements. In addition to receptor ligation, ncNF-κB can be activated by signaling intermediates of the apoptosis cascade. Cleavage of the second mitochondrial activator of caspases (SMAC) from the mitochondrial membrane exposes the N-terminal motif Ala-Val-Pro-Ile, which binds specifically to the baculovirus intermediate repeat (BIR) domains of the IAP proteins. Such BIR binding in cIAP1/2 activates the ubiquitin ligase activity of the TRAF2:TRAF3:cIAP complex, inducing autoubiquitinylation and degradation of cIAP1/2, NIK accumulation, and activation of the ncNF-κB pathway [Sampey bioRxiv 2018][Nixon Nature 2020]. Binding of SMAC to the BIR domains of XIAP and ML-IAP antagonizes the caspase inhibition activities of these molecules, often overexpressed in tumor cells, leading to potentiation of apoptosis.

Accordingly, the discovery and development of new SMACm molecules represent a currently unmet medical need.

### SUMMARY OF THE INVENTION

The present invention relates to compounds according to Formula (I):

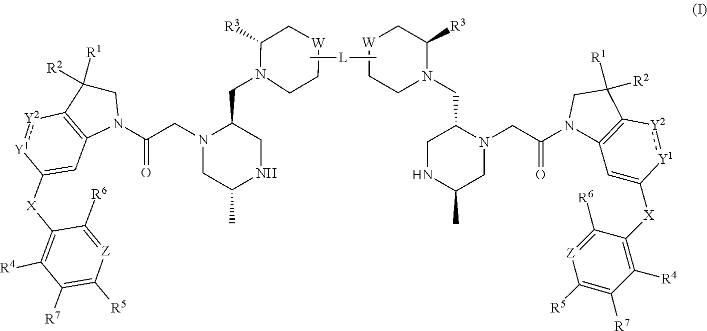

or a pharmaceutically acceptable salt thereof, wherein:
each R¹ and R² is —H, or —CH₃;
each R³ is —H or —CH₃;
each R⁴ is —H, —F, —Cl, —CH₃, —CF₃, —CN, —OH, —OCH₃, —C(O)N(CH₃)₂, —CH(CH₃)₂, or —C(O)OCH₃;
each R⁵ is —H, —F, —Cl, —Br, —CH₃, —CHF₂, or —CF₃;
each R⁶ is —H, —F, or —Cl;
each R⁷ is —H or —F;
each W is —CH—, —CH₂—, —O—, or —N—;
each X is —CH₂—, —O—, —NH—, or —NCH₃—;
each Y¹ is —CH— or —C(O)—;
each Y² is —N—, —NH—, or —NCH₃—;
each [symbol] represents a single or double bond, wherein when Y¹ is —CH—, Y² is —N- and [symbol] represents a double bond and when Y¹ is —C(O)—, Y² is —NH— or —NCH₃—, and [symbol] represents a single bond;
each Z is —CH— or —N—; and
L is a linker selected from the group consisting of

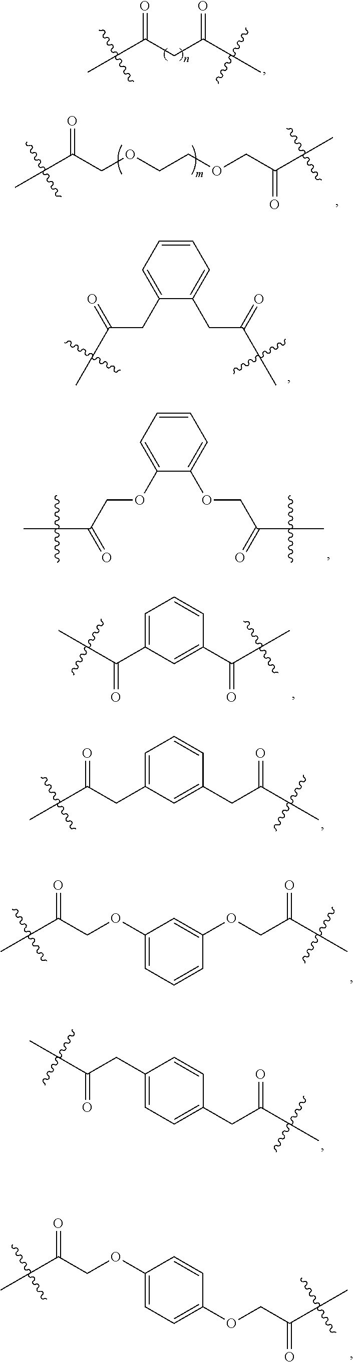

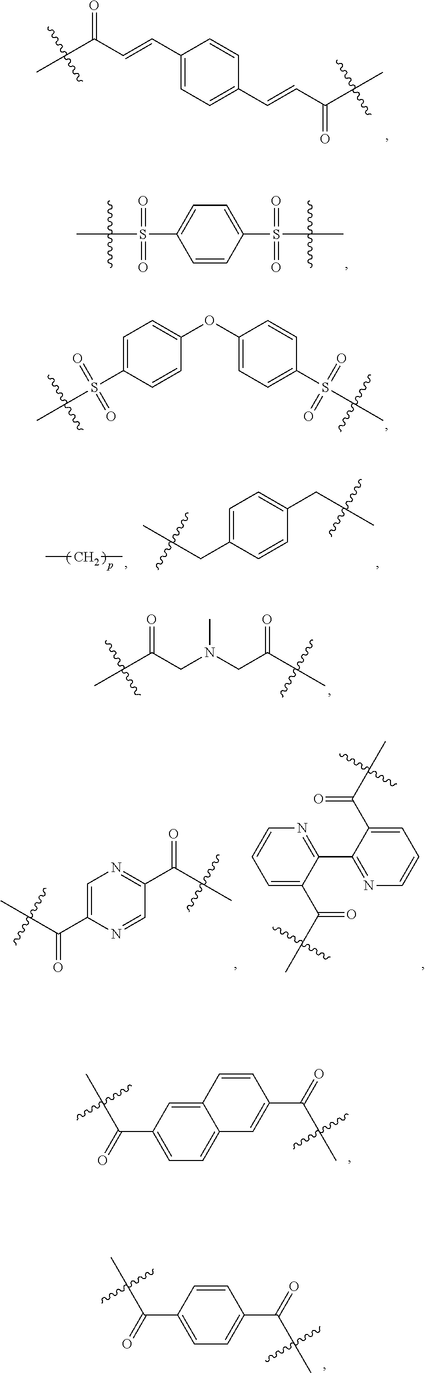

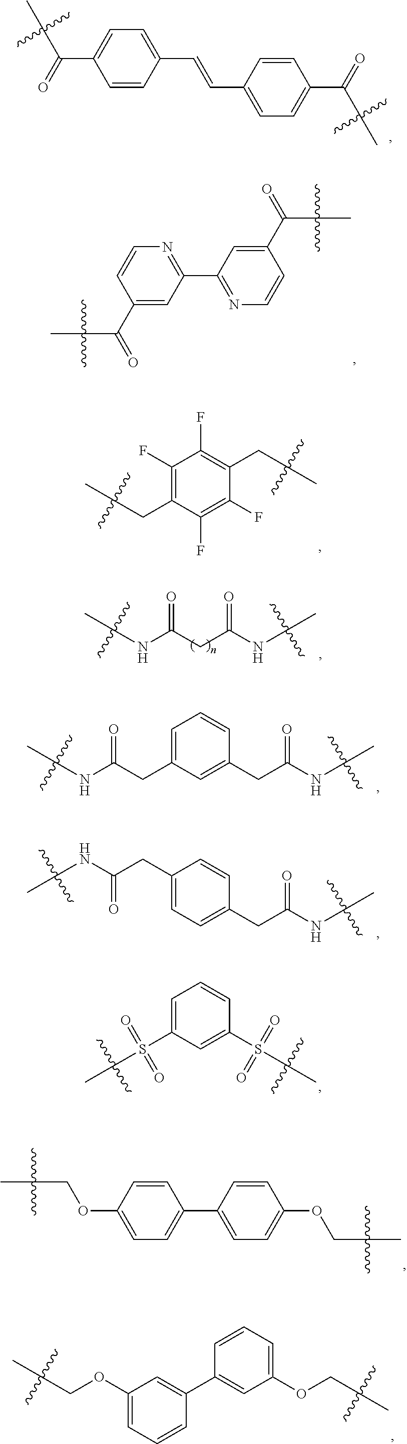

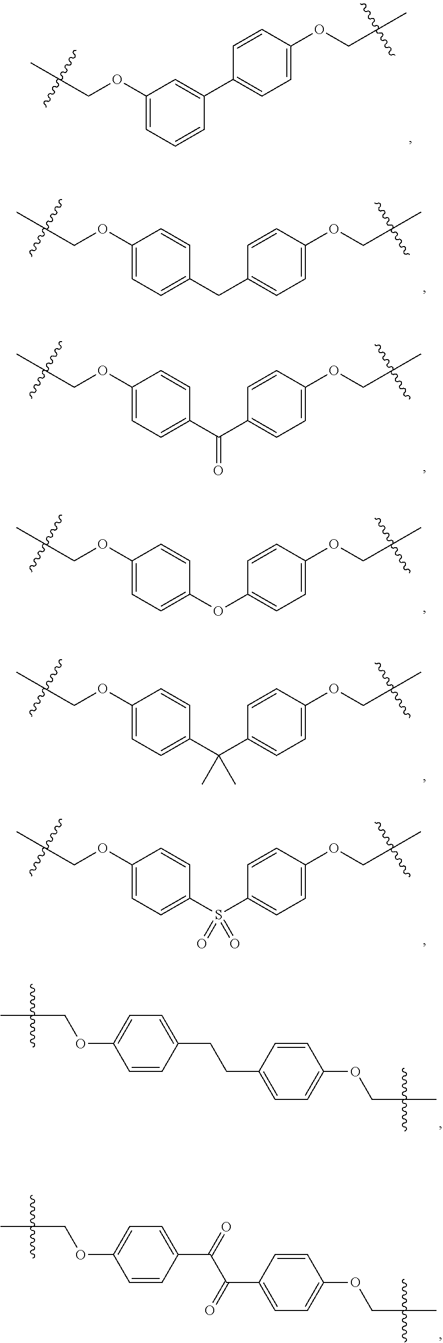

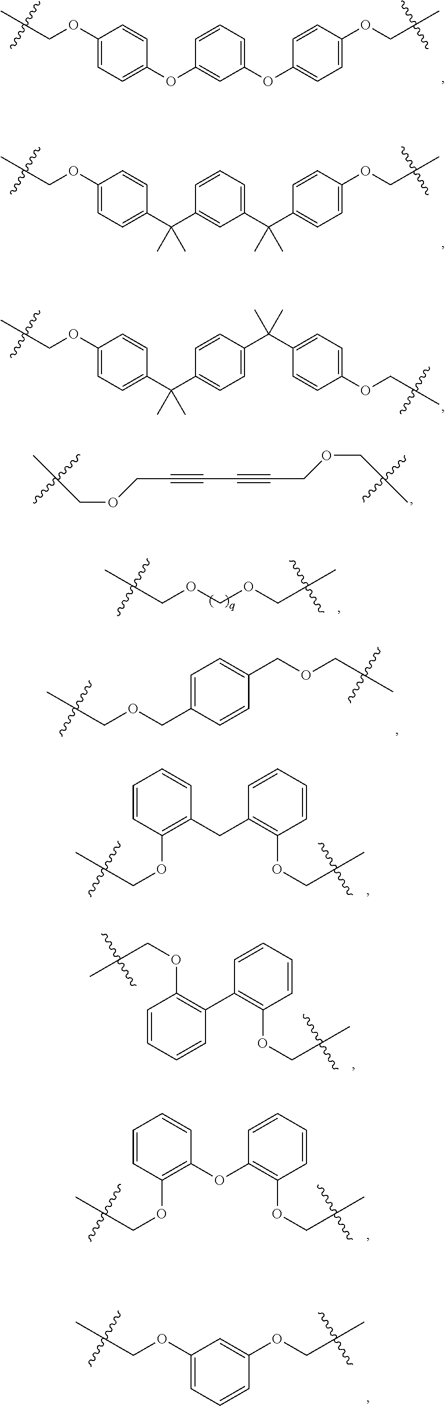

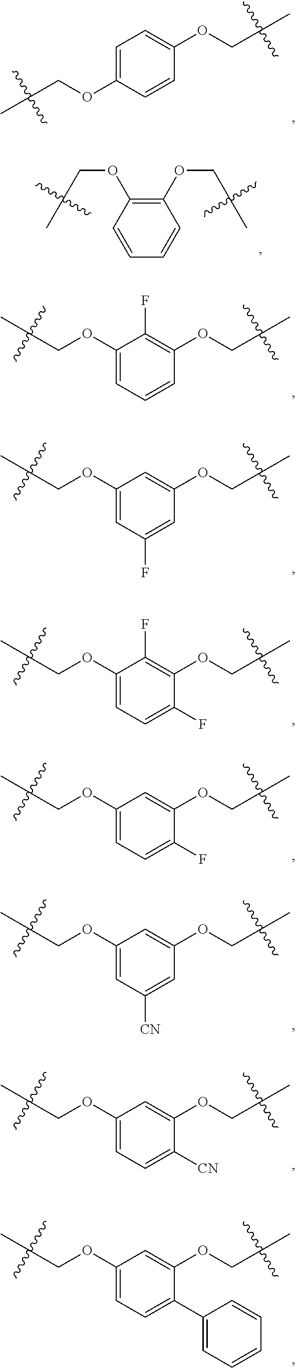

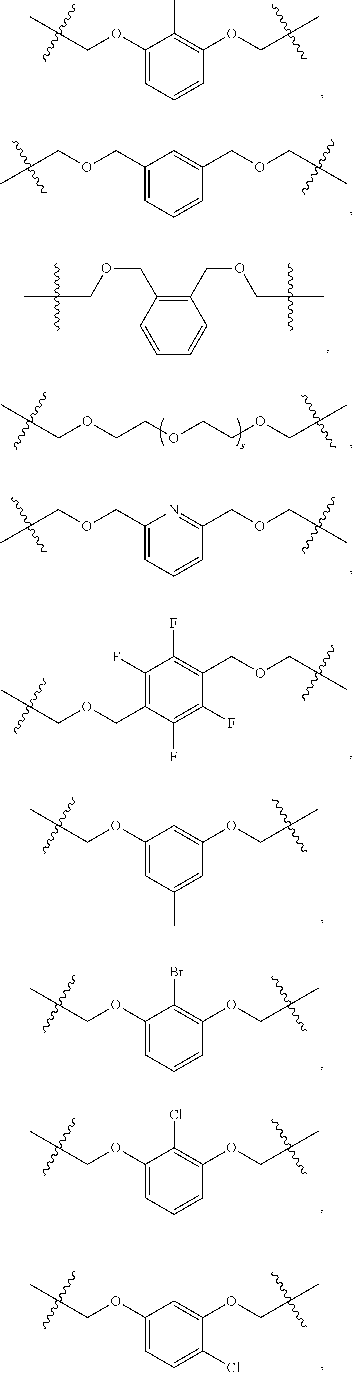

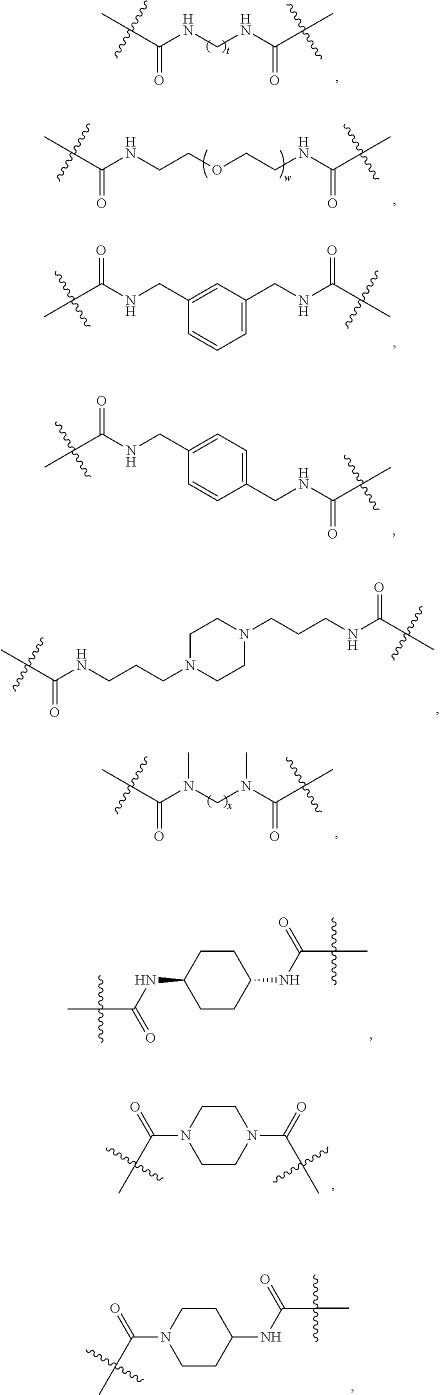

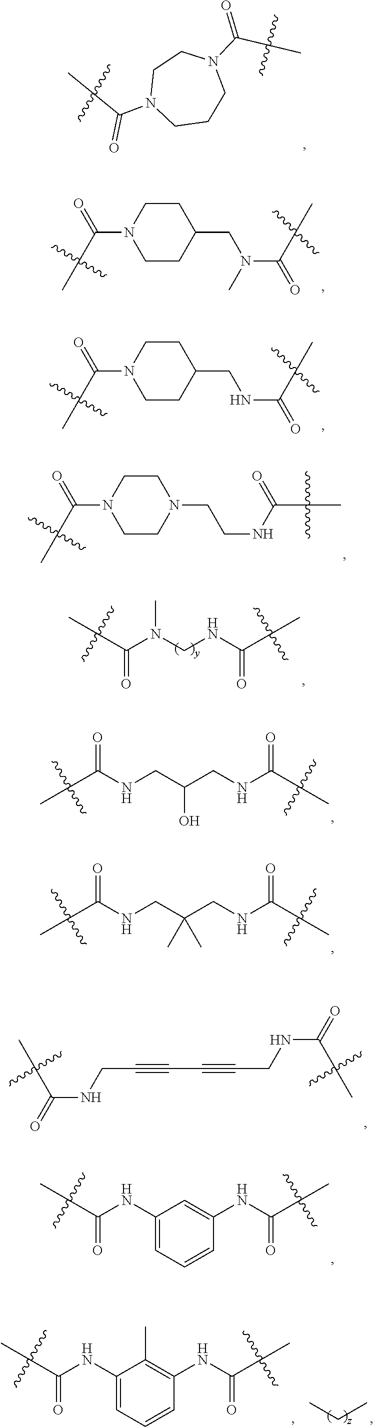

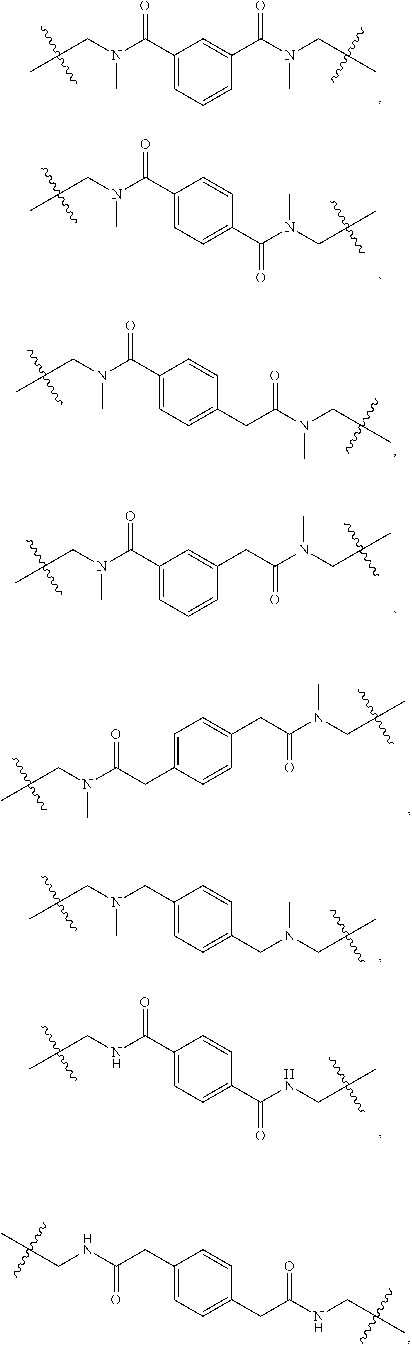

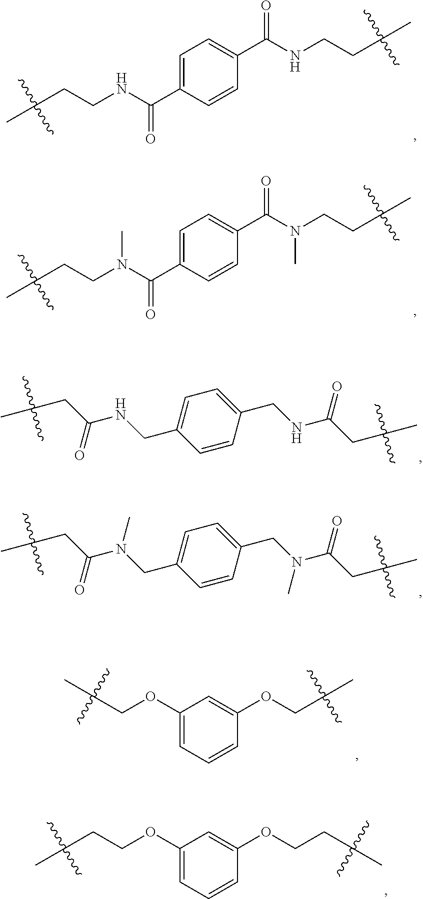 [SMILES: *CCNC(=O)c1ccc(C(=O)NCC*)cc1 ; *CCN(C)C(=O)c1ccc(C(=O)N(C)CC*)cc1 ; *CC(=O)NCc1ccc(CNC(=O)C*)cc1 ; *CC(=O)N(C)Cc1ccc(CN(C)C(=O)C*)cc1 ; *COc1cccc(OC*)c1 ; *CCOc1cccc(OCC*)c1]

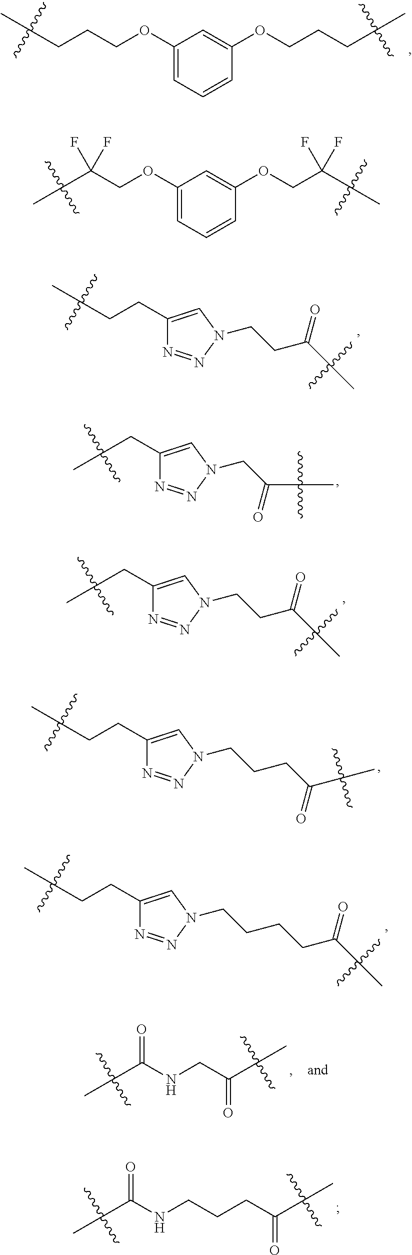

wherein [symbol] indicates a point of attachment; and wherein n is an integer from 2 to 15, m is an integer from 1 to 5, p is an integer from 1 to 20, q is an integer from 2 to 15, s is an integer from 1 to 8, t is an integer from 2 to 15, w is an integer from 1 to 10, x is an integer from 2 to 15, y is an integer from 2 to 15, and z is an integer from 2 to 15.

Another aspect of the invention provides a pharmaceutical composition comprising a compound according to Formula (I) or a pharmaceutically acceptable salt thereof and a pharmaceutically acceptable excipient.

In another aspect, the invention provides a method of treating an HIV infection in a human comprising administering to the human a therapeutically effective amount of a compound according to Formula (I) or pharmaceutically acceptable salt thereof, or a pharmaceutical composition comprising a compound according to Formula (I) or a pharmaceutically acceptable salt thereof and a pharmaceutically acceptable excipient.

In yet another aspect, the invention provides a use of a compound according to Formula (I) or pharmaceutically acceptable salt thereof, or a pharmaceutical composition comprising a compound according to Formula (I) or a pharmaceutically acceptable salt thereof and a pharmaceutically acceptable excipient, in the manufacture of a medicament for treating an HIV infection.

In still another aspect, the invention provides a method of treating cancer and pre-cancerous syndromes, in a human in need thereof, which comprises administering to the human a therapeutically effective amount of a compound according to Formula (I) or pharmaceutically acceptable salt thereof, or a pharmaceutical composition comprising a compound according to Formula (I) or a pharmaceutically acceptable salt thereof and a pharmaceutically acceptable excipient.

In another aspect, the invention provides a method of depleting HIV infected cells comprising administering to a subject a compound of Formula (I) or pharmaceutically acceptable salt thereof, or a pharmaceutical composition comprising a compound according to Formula (I) or a pharmaceutically acceptable salt thereof and a pharmaceutically acceptable excipient.

In still aspect, the invention provides a combination comprising a compound of Formula (I) or pharmaceutically acceptable salt thereof, and one or more pharmaceutical agents active against HIV. In certain aspects, these pharmaceutical agents active against HIV are selected from the group consisting of anti-retroviral agents, latency reversing agents, and agents for clearance therapy.

In yet another aspect, the invention provides a method of depleting HIV infected cells comprising administering to a subject a compound of Formula (I), or pharmaceutically acceptable salt thereof, or a pharmaceutical composition comprising a compound according to Formula (I) or a pharmaceutically acceptable salt thereof and a pharmaceutically acceptable excipient and one or more additional agents active against HIV. In certain aspects, these pharmaceutical agents active against HIV are selected from the group consisting of anti-retroviral agents, latency reversing agents, and agents for clearance therapy.

These and other aspects are encompassed by the invention as set forth herein.

### BRIEF DESCRIPTION OF THE FIGURES

FIG. 1 is a graph comparing rodent pharmacokinetic (PK) data of several compounds of Formula I with that of SMACm AZD5582 PK data; and

FIG. 2 is a table summarizing the activity and selectivity of certain SMAC mimetics disclosed herein with other mimetics.

### DETAILED DESCRIPTION

### Definitions

It is to be understood that the terminology used herein is for the purpose of describing particular embodiments only and is not intended to limit the scope of the present disclosure. In this specification and in the claims that follow, reference will be made to a number of terms that shall be defined to have the following meanings.

“Alkyl” refers to a saturated, straight or branched hydrocarbon moiety having from 1 to 6 carbon atoms unless specified otherwise. The term “(C₁-C₆) alkyl” refers to an alkyl moiety containing from 1 to 6 carbon atoms. Examples of “alkyl” as used herein include, but are not limited to, methyl, ethyl, n-propyl, isopropyl, n-butyl, isobutyl, s-butyl, t-butyl, pentyl, and hexyl.

“Alkylene” refers to a straight or branched chain divalent alkyl radical having from 1 to 6 carbon atoms unless specified otherwise. The term “(C₁-C₆) alkylene” refers to an alkylene containing from 1 to 6 carbon atoms. Examples of “alkylene” as used herein include, but are not limited to, methylene, ethylene, n-propylene, n-butylene, and die like.

“Substituted alkylene” refers to an alkylene having from 1 to 5 and, in some embodiments, 1 to 3 or 1 to 2 substituents selected from alkoxy, substituted alkoxy, acyl, acylamino, acyloxy, alkyl, substituted alkyl, alkoxy, amino, substituted amino, quaternary amino, aminocarbonyl, imino, amidino, aminocarbonylamino, amidinocarbonylamino, aminothiocarbonyl, aminocarbonylamino, aminothiocarbonylamino, aminocarbonyloxy, aminosulfonyl, aminosulfonyloxy, aminosulfonylamino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, phosphate, phosphonate, phosphinate, phosphonamidate, phosphorodiamidate, phosphoramidate monoester, cyclic phosphoramidate, cyclic phosphorodiamidate, phosphoramidate diester, sulfate, sulfonate, sulfonyl, substituted sulfonyl, sulfonyloxy, thioacyl, thiocyanate, thiol, alkylthio, and substituted alkylthio. It is understood that the above definition is not intended to include impermissible substitution patterns.

“Alkoxy” refers to a straight or branched alkoxy group having from 1 to 6 carbon atoms unless specified otherwise. For example, “C₁-C₆ alkoxy” means a straight or branched alkoxy group containing from 1 to 6 carbon atoms. Examples of “alkoxy” as used herein include, but are not limited to, methoxy, ethoxy, prop-1-oxy, prop-2-oxy, but-1-oxy, but-2-oxy, 2-methylprop-I-oxy, 2-methylprop-2-oxy, pentoxy and hexyloxy.

“Aryl” or “Ar” refers to an aromatic hydrocarbon ring. “Aryl” includes monocyclic, bicyclic, and tricyclic ring systems having a total of 5 to 15 ring member atoms unless specified otherwise, wherein at least one ring system is aromatic and wherein each ring in the system contains 3 to 7 member atoms “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system Examples of “alkoxy” as used herein include, but are not limited to, phenyl, naphthyl, indenyl, azulenyl, fluorenyl, anthracenyl, phenanthrenyl, tetrahydronaphthyl, indanyl, phenanthridinyl and the like.

“Arylene” refers to a divalent aromatic hydrocarbon ring. “Arylene” includes monocyclic, bicyclic, and tricyclic ring systems having a total of 5 to 15 ring member atoms unless specified otherwise, wherein at least one ring system is aromatic and wherein each ring in the system contains 3 to 7 member atoms. “Arylene” also includes ring systems wherein the aryl ring, as defined above, is fused with one or more carbocyclyl or heterocyclyl groups wherein the radicals or points of attachment are on the aryl ring, and in such instances, the number of carbon atoms continue to designate the number of carbon atoms in the aryl ring system Examples of“arylene” as used herein include or are derived from, but are not limited to, phenylene (para, meta, ortho), naphthalene, biphenyl, indole, triazole, and the like.

“Substituted arylene” refers to an arylene substituted with 1 to 8 and, in some embodiments, 1 to 5, 1 to 4, 1 to 3, or 1 to 2 substituents selected from alkyl, substituted alkyl, alkenyl, substituted alkenyl, alkynyl, substituted alkynyl, alkoxy, substituted alkoxy, acyl, acylamino, acyloxy, amino, substituted amino, quaternary amino, aminocarbonyl, imino, amidino, aminocarbonylamino, amidinocarbonylamino, aminothiocarbonyl, aminocarbonylamino, aminothiocarbonylamino, aminocarbonyloxy, aminosulfonyl, aminosulfonyloxy, aminosulfonylamino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, phosphate, phosphonate, phosphinate, phosphonamidate, phosphorodiamidate, phosphoramidate monoester, cyclic phosphoramidate, cyclic phosphorodiamidate, phosphoramidate diester, sulfate, sulfonate, sulfonyl, substituted sulfonyl, sulfonyloxy, thioacyl, thiocyanate, thiol, alkylthio, and substituted alkylthio. It is understood that the above definition is not intended to include impermissible substitution patterns.

“Compound” and “compounds” as used herein refers to a compound encompassed by the generic formulae disclosed herein, any subgenus of those generic formulae, and any forms of the compounds within the generic and subgeneric formulae, including the racemates, stereoisomers, and tautomers of the compound or compounds.

“Cyano” refers to a —C≡N functional group.

“Cycloalkylene” refers to a non-aromatic, saturated, cyclic divalent hydrocarbon ring containing 3 to 7 member ring atoms unless otherwise specified. “Heterocycloalkylene” refers to a “cycloalkylene” wherein at least one ring atom is a heteroatom. Examples of “cycloalkylene” as used include or are derived from, but are not limited to, cyclopropyl, cyclobutyl, cyclopentyl, cyclobexyl, and piperidine.

“Substituted cycloalkylene” refers to a cycloalkylene, as defined herein, having from 1 to 8, or 1 to 5, or in some embodiments 1 to 3 substituents selected from alkyl, substituted alkyl, alkenyl, substituted alkenyl, alkynyl, substituted alkynyl, alkoxy, substituted alkoxy, acyl, acylamino, acyloxy, amino, substituted amino, quaternary amino, aminocarbonyl, imino, amidino, aminocarbonylamino, amidinocarbonylamino, aminothiocarbonyl, aminocarbonylamino, aminothiocarbonylamino, aminocarbonyloxy, aminosulfonyl, aminosulfonyloxy, aminosulfonylamino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, phosphate, phosphonate, phosphinate, phosphonamidate, phosphorodiamidate, phosphoramidate monoester, cyclic phosphoramidate, cyclic phosphorodiamidate, phosphoramidate diester, sulfate, sulfonate, sulfonyl, substituted sulfonyl, sulfonyloxy, thioacyl, thiocyanate, thiol, alkylthio, and substituted alkylthio. The term “substituted cycloalkyl” includes substituted cycloalkenyl groups. It is understood that the above definition is not intended to include impermissible substitution patterns.

“Halo” or “halogen” refers to a fluorine (fluoro, F), chlorine (chloro, Cl), bromine (bromo, Br) or iodine (iodo, I).

“Heteroatom” refers to nitrogen, oxygen, or sulfur,

“Latency” means a concept describing 1) the dormant state of viral activity within a population of cells, wherein viral production, viral packaging, and host cell lysis does not occur, or occurs at a very low frequency, or 2) the down-regulation or absence of gene expression within an infected cell.

“Optionally” means that the subsequently described event(s) may or may not occur and includes both event(s) that occur and event(s) that do not occur.

“Solvate” or “solvates” of a compound refer to those compounds, where compounds is as defined above, that are bound to a stoichiometric or non-stoichiometric amount of a solvent. Solvates of a compound include solvates of all forms of the compound. In some embodiments, solvents are volatile, non-toxic, and/or acceptable for administration to humans in trace amounts. Suitable solvents include water.

“Stereoisomer” refers to compounds that differ in the chirality of one or more stereocenters. Stereoisomers include enantiomers and diastereomers. The compounds of the Formula (I) contain at least one asymmetric center (also referred to as a chiral center or stereocenter) and may therefore exist as individual enantiomers (also known as optical isomers), diastereomers, or other stereoisomeric forms, such as epimers, or as mixtures thereof. Chiral centers, such as chiral carbon atoms, may also be present in a substituent such as an alkyl group in which a carbon atom is attached to four different groups. Where the stereochemistry of a chiral center present in a compound of the invention or in any other chemical structure illustrated herein is not specified, the structure is intended to encompass any individual stereoisomer and also all mixtures thereof.

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N-moiety such as pyrazoles, imidazoles, benzimidazoles, triazoles, and tetrazoles. The compounds of the invention may exist in tautomeric forms. It is to be understood that any reference to a named compound or a structurally depicted compound is intended to encompass all tautomers of such compound. For example, in the embodiments disclosed herein, the compounds may exist in either the pyrrolopyridone or pyrrolohydroxypyridine tautomeric form or in any stoichiometric combination of the respective tautomers:

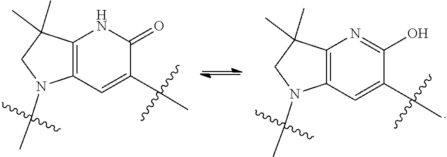 [SMILES: *c1cc2c([nH]c1=O)C(C)(C)CN2* ; *c1cc2c(nc1O)C(C)(C)CN2*]

“Pharmaceutically acceptable salt” refers to pharmaceutically acceptable salts derived from pharmaceutically acceptable counterions. Suitable salts include those described in P. Heinrich Stahl, Camille G. Wermuth (Eds.), Handbook of Pharmaceutical Salts Properties, Selection, and Use; 2002. It will be understood that if a compound of Formula (I) contains two or more basic moieties, the stoichiometry of salt formation may include 1, 2 or more equivalents of acid (i.e., acid addition salt). Such salts would contain 1, 2 or more acid counterions, for example, a dihydrochloride salt. Stoichiometric and non-stoichiometric forms of a pharmaceutically acceptable salt of a compound of Formula (I) are included within the scope of the invention, including sub-stoichiometric salts, for example where a counterion contains more than one acidic proton. Representative pharmaceutically acceptable acid addition salts include, but are not limited to, 4-acetamidobenzoate, acetate, adipate, alginate, ascorbate, aspartate, benzenesulfonate (besylate), benzoate, bisulfate, bitartrate, butyrate, calcium edetate, camphorate, camphorsulfonate (camsylate), caprate (decanoate), caproate (hexanoate), caprylate (octanoate), cinnamate, citrate, cyclamate, digluconate, 2,5-dihydroxybenzoate, disuccinate, dodecylsulfate (estolate), edetate (ethylenediaminetetraacetate), estolate (lauryl sulfate), ethane-1,2-disulfonate (edisylate), ethanesulfonate (esylate), formate, fumarate, galactarate (mucate), gentisate (2,5-dihydroxybenzoate), glucoheptonate (gluceptate), gluconate, glucuronate, glutamate, glutarate, glycerophosphorate, glycolate, hexylresorcinate, hippurate, hydrabamine (N,N′-di(dehydroabietyl)-ethylenediamine), hydrobromide, hydrochloride, hydroiodide, hydroxynaphthoate, isobutyrate, lactate, lactobionate, laurate, malate, maleate, malonate, mandelate, methanesulfonate (mesylate), methylsulfate, mucate, naphthalene-1,5-disulfonate (napadisylate), naphthalene-2-sulfonate (napsylate), nicotinate, nitrate, oleate, palmitate, p-aminobenzenesulfonate, p-aminosalicyclate, pamoate (embonate), pantothenate, pectinate, persulfate, phenylacetate, phenylethylbarbiturate, phosphate, polygalacturonate, propionate, p-toluenesulfonate (tosylate), pyroglutamate, pyruvate, salicylate, sebacate, stearate, subacetate, succinate, sulfamate, sulfate, tannate, tartrate, teoclate (8-chlorotheophyllinate), thiocyanate, triethiodide, undecanoate, undecylenate, and valerate.

‘Pharmaceutically acceptable’ refers to those compounds (including salts), materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

“Reversing HIV latency” refers to a treatment that upregulates the expression of integrated HIV genomes within latently infected cells, such as the agent that activates the non-canonical NF-kB pathway, leading to susceptibility of the infected cell to virally-induced cell death or immunologic clearance. In certain embodiments, the latent HIV infected cells are resting CD4⁺ T cells. As used herein, “depleting latent HIV infection” refers to the clearance of latently HIV-infected cells that may follow the reversal of HIV latency by reagents such as those that activate the non-canonical NF-kB pathway. In some embodiments, each of compounds 1-112 may be used in reversing HIV latency and/or depleting latent HIV infection.

“Therapeutically effective amount” means any amount which, as compared to a corresponding subject who has not received such amount, results in improved treatment, healing, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. Additionally, the active ingredient may be presented as a pharmaceutical composition.

“Treating viral infections” means to inhibit the replication of the particular virus, to inhibit viral transmission, and to ameliorate or alleviate the symptoms of the disease caused by the viral infection. The treatment is considered “therapeutic” if there is a reduction in viral load, decrease in mortality and/or morbidity. “Preventing viral infections” means to prevent the virus from establishing itself in the host. A treatment is considered “prophylactic” if the subject is exposed to the virus but does not become infected with the virus as a result of treatment.

Wherever dashed lines occur adjacent to single bonds denoted by solid lines, then the dashed line represents an optional double bond at that position. Wherever wavy lines “ [symbol] ” occur across a bond, “ [symbol] ” indicates a point of attachment. For example, the two wavy lines in the structure below indicate two different points of attachment to additional chemical moieties, and the dashed line in the structure below could either indicate a double bond at that position or a single bond at that position:

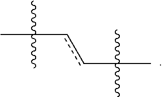

The presently disclosed subject matter will now be described more fully hereinafter. However, many modifications and other embodiments of the presently disclosed subject matter set forth herein will come to mind to one skilled in the art to which the presently disclosed subject matter pertains having the benefit of the teachings presented in the foregoing descriptions. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

Apoptosis, a type of programmed cell death, plays an important role in maintaining homeostasis and regulating the number of cells in higher organisms. Abnormal apoptosis is involved in a number of diseases, including autoimmune disorders, degenerative diseases of the Central Nervous System, cancer, and viral infections, such as HIV. The family of Inhibitor of Apoptosis Proteins (IAPs) plays a key role in the suppression of proapoptotic signaling in mammalian cells. SMACm, which mimic a critical tetrapeptide sequence from the second mitochondria-derived activator of caspase, have been shown to disrupt the binding of IAPs with their functional partner and restore apoptotic response to proapoptotic stimuli in cells. Since the early 2000s, great effort has focused on the design and preparation of SMAC mimetics as IAP antagonists, particularly in promoting cell death in tumor cells and more recently in reversing HIV latency. SMAC mimetics bind the BIR2 and BIR3 domains of IAP proteins, leading to inhibition (in the case of XIAP) and potentiation of apoptosis, or activation of the ubiquitin ligase activities (in the case of cIAP1 and cIAP2), leading to autoubiquitinylation and depletion of the activated proteins. As cIAP1 and 2 suppress the ncNF-kB pathway, it is observed that the activation of these proteins by SMACm leads to activation of ncNFkB transcription factor and leads to kB-dependent gene expression. Such investigations have explored the activation of the non-canonical NF-kB pathway (ncNF-kB) as a potential method by which SMAC mimics selectively deplete latent HIV cells. An example of an early SMAC mimetic studied in the context of HIV is monomeric SBI-0637142, prepared by researchers at the Sanford-Burnham Medical Research Institute. In HIV depletion tests, SBI-0637142 was found to be potent in cell line assays, but did not exhibit activity in p100-p52 conversion or HIV caRNA induction in primary cells. Much work has also been directed to the development of bivalent mimetics in the oncology field, which are covalently linked monomeric SMAC mimetics. AstraZeneca's AZD5582 and Medivir's Birinapant TL32711 are examples of dimeric SMAC mimetics. In HIV latency reversal studies, Birinapant TL32711 was not potent in Jurkat, p100-p52 conversion, or HIV caRNA induction. Conversely, AZD5582 exhibited an increase in cell-associated HIV RNA expression in resting CD4+ T cells through Jurkat assay experiments, p100-p52 conversion studies, and HIV cell-associated RNA induction (Sampey et al. bioRxiv 312447). However, AZD5582 can also demonstrate tolerability issues.

The IAP proteins targeted by SMAC mimetics for ncNF-kB activation are cIAP1 and cIAP2, with bivalent molecules possessing optimal linkers having the most efficient activity at depleting these two molecules and activating ncNF-kB. This ncNF-kB activating activity occurs perhaps through forming intermolecular ternary complexes wherein one bivalent SMAC mimetic interacts with one BIR domain in each of two different molecules of cIAP1 or cIAP2. XIAP is also bound and inhibited by SMAC mimetics, described in the literature as an intramolecular bond where one bivalent SMAC mimetic interacts with both the BIR2 and BIR3 domains of a single molecule of XIAP. XIAP is not thought to have a role in ncNF-kB activation by SMAC mimetics and is therefore likely an off target in the HIV latency reversal context.

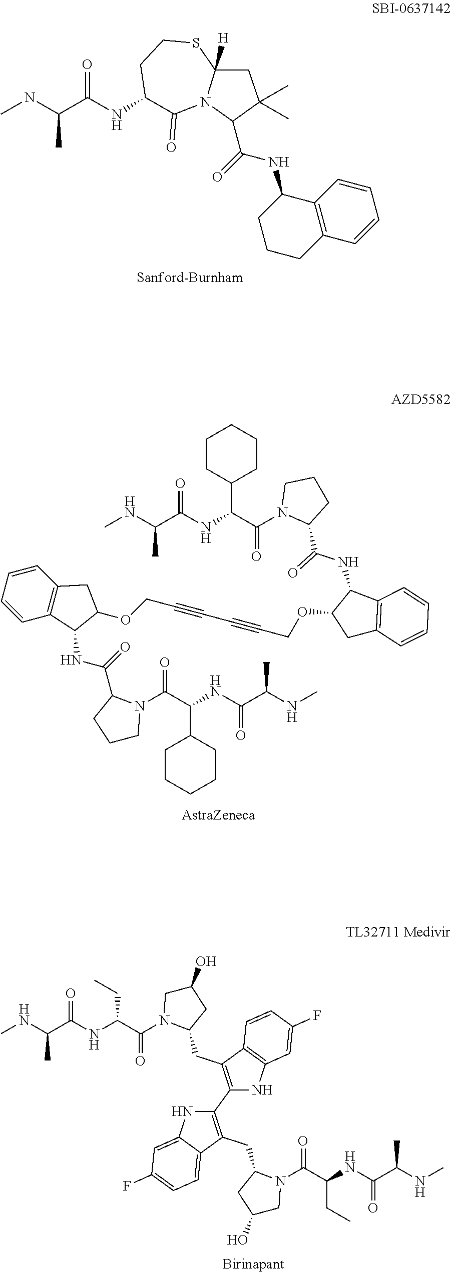

Disclosed herein are dimeric SMACm sufficiently potent and effective enough to activate ncNF-kB, reverse HIV latency in primary, unmodified primary human cells as single agents, making them suitable for consideration for further development. In particular, the dimeric SMACm of the disclosed compounds are optimized to favor binding of BIR3 over BIR2 especially those domains within XIAP, which should favor intermolecular ternary complex formation between two IAP proteins and therefore favor the depletion of cIAP1 and cIAP2 over inhibition of XIAP. Other SMACm, specifically monomeric molecules or dimeric molecules with unoptimized linkers or lacking this specificity for BIR3 over BIR2, are not believed to have the HIV latency reversal effect in primary human CD4+ T cells and likely also inhibit XIAP, leading to potential off-target effects through potentiation of unwanted apoptosis.

The invention provides compounds of Formulas (I), as well as various forms of these compounds set forth herein (e.g., pharmaceutically acceptable salts, tautomers, and stereoisomers). It should be appreciated that any reference to the compounds of Formulas (I) herein is clearly meant to also include, without limitation, those compounds set forth in Table 1.

In some embodiments, the invention provides a compound of the structure according to Formula (I):

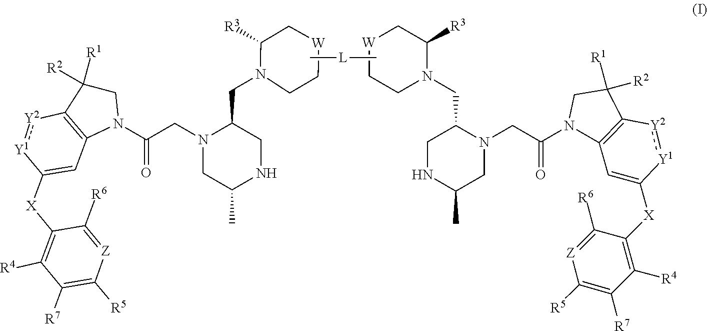

or a pharmaceutically acceptable salt thereof, or in other embodiments a compound of the structure according to Formula (Ia):

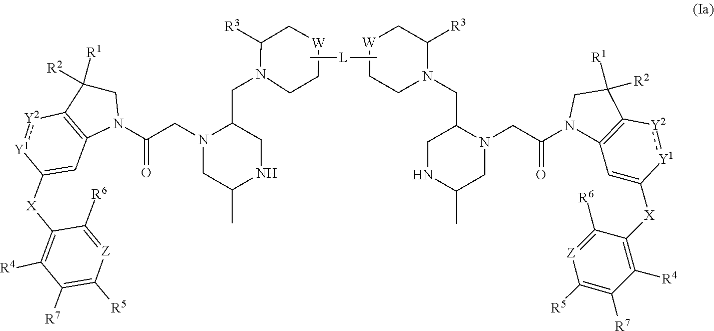

or a pharmaceutically acceptable salt or stereoisomer thereof; wherein:
each R¹ and R² is —CH₃;
each R³ is —H or —CH₃;
each R⁴ is —H, —F, —Cl, —CH₃, —CF₃, —CN, —OH, —OCH₃, —C(O)N(CH₃)₂, —CH(CH₃)₂, or —C(O)OCH₃;
each R⁵ is —H, —F, —Cl, —Br, —CH₃, —CHF₂, or —CF₃; each R⁶ is —H, —F, or —Cl;
each R⁷ is —H or —F;
each W is —CH—, —CH₂—, —O—, or —N—;
each X is —CH₂—, —O—, —NH—, or —NCH₃—;
each Yⁱ is —CH— or —C(O)—;
each Y² is —N—, —NH—, or —NCH₃—;
each [symbol] represents a single or double bond, wherein when Y¹ is —CH—, Y² is —N- and [symbol] represents a double bond and when Yⁱ is —C(O)—, Y² is —NH— or —NCH₃—, and [symbol] represents a single bond;
each Z is —CH— or —N—; and
L is a linker selected from the group consisting of

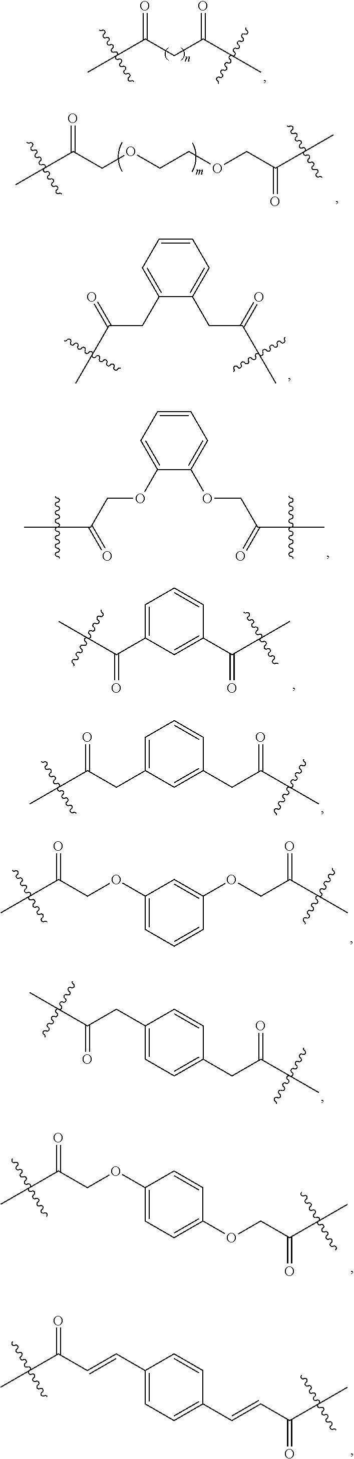

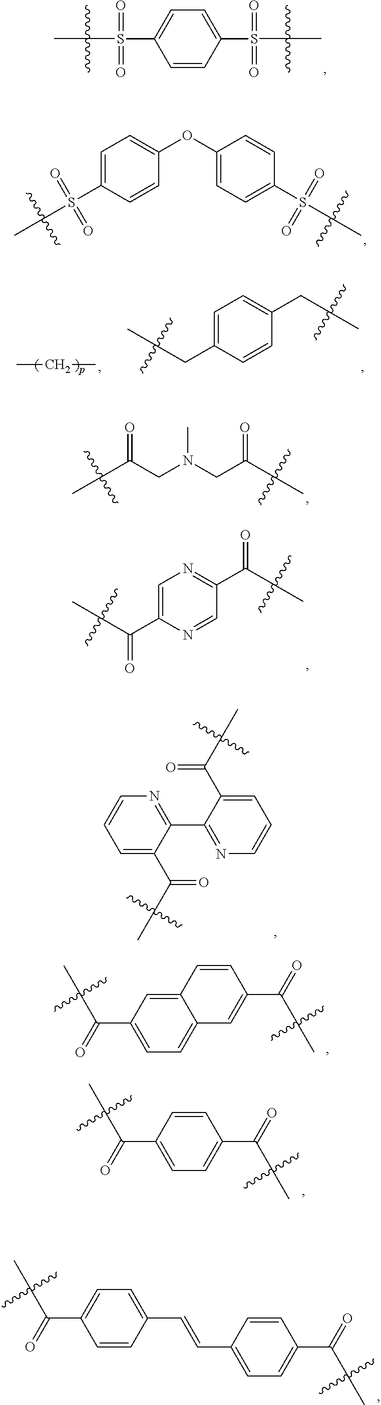

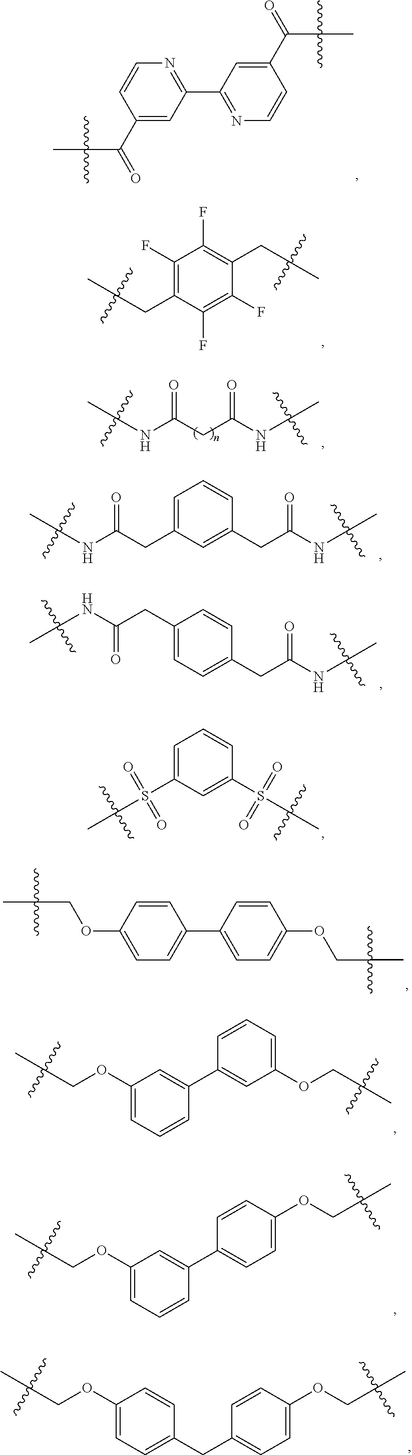

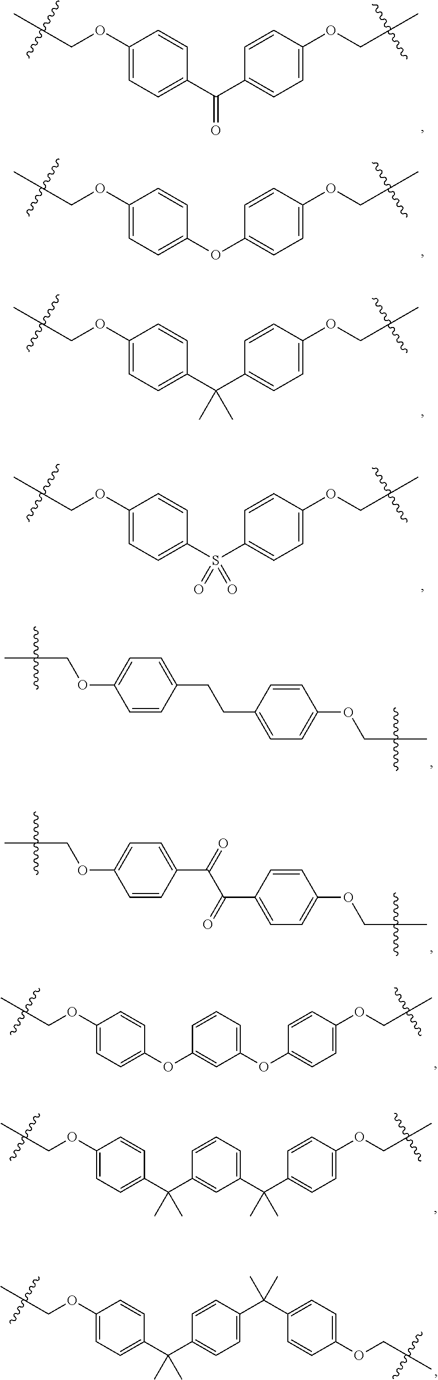

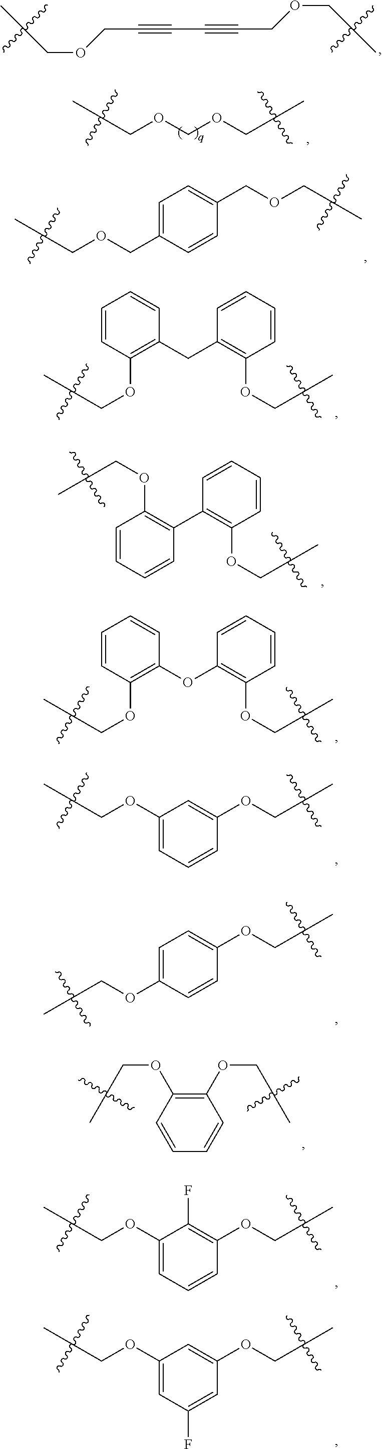

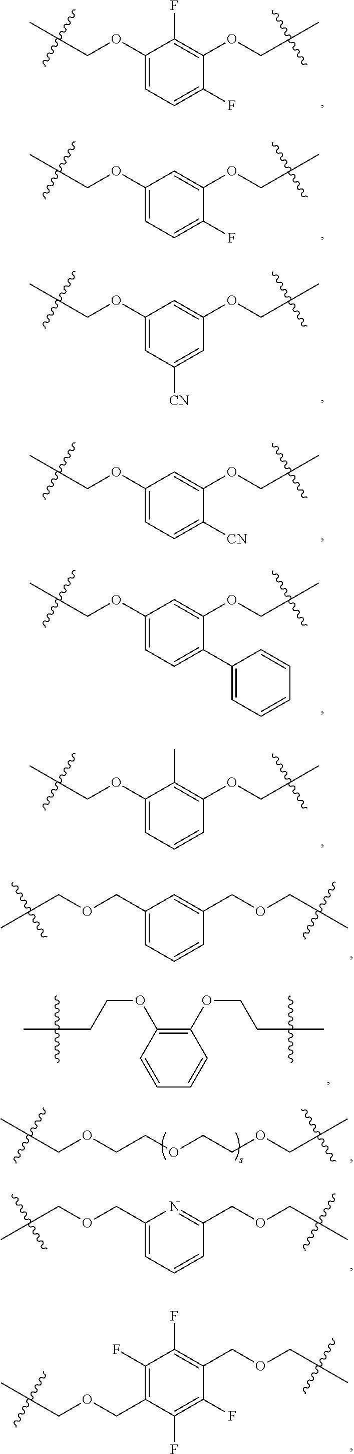

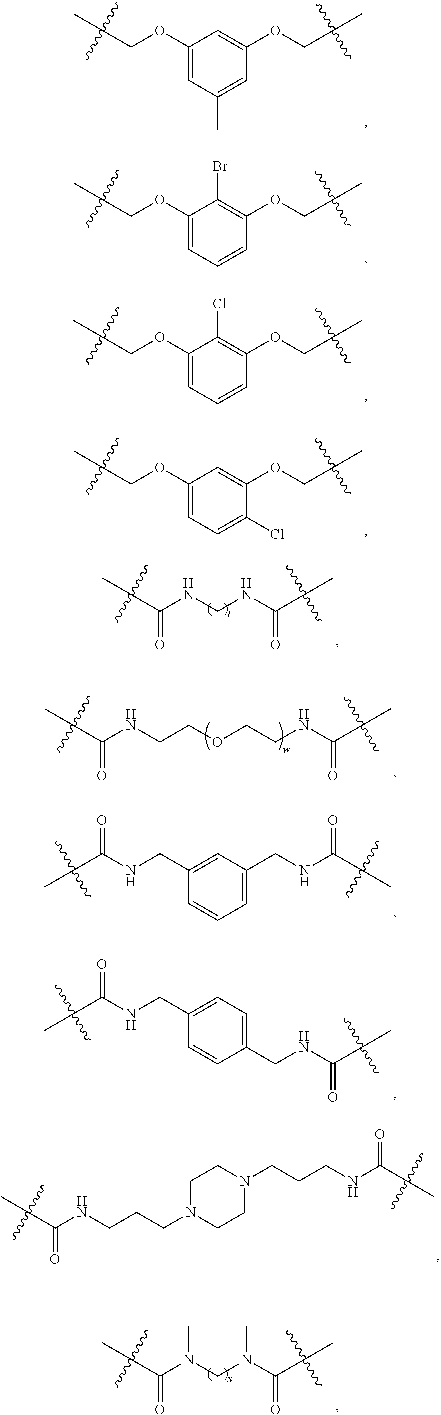

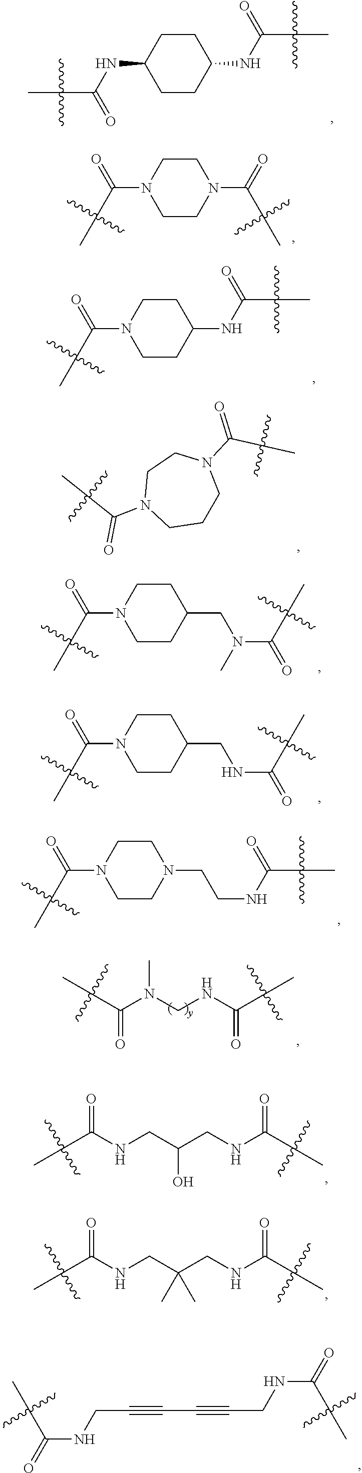

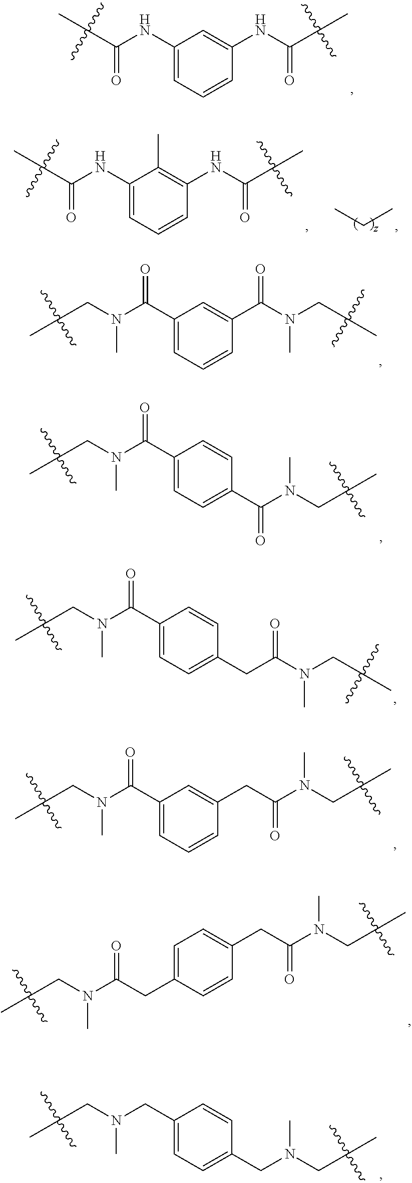

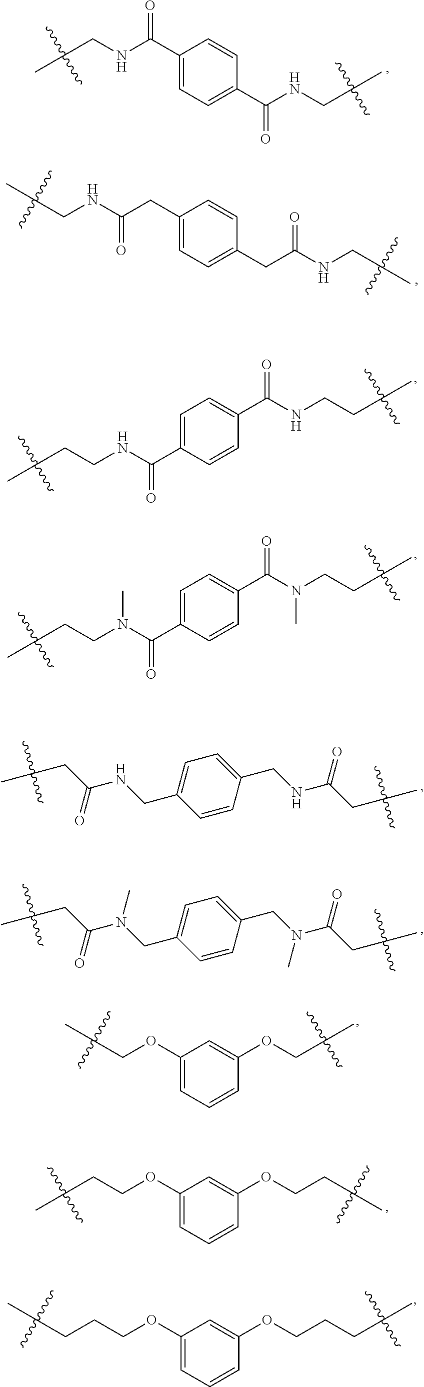

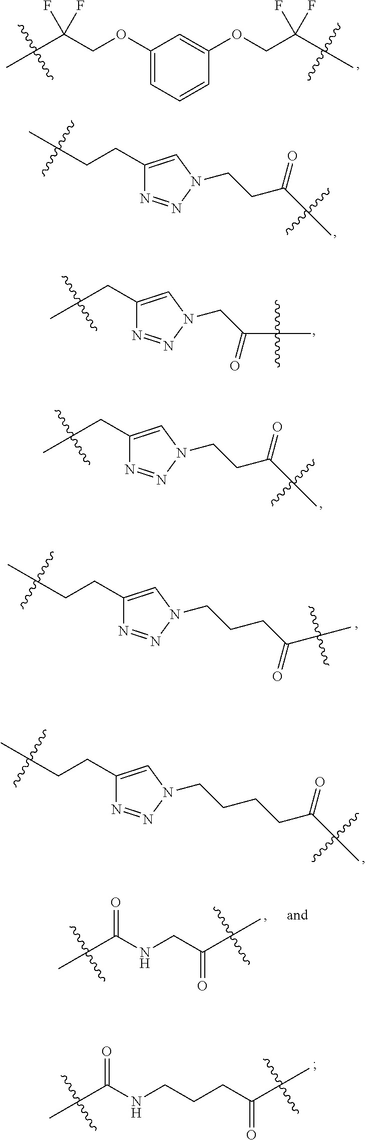

wherein [symbol] indicates a point of attachment; and wherein n is an integer from 2 to 15, m is an integer from 1 to 5, p is an integer from 1 to 20, q is an integer from 2 to 15, s is an integer from 1 to 8, t is an integer from 2 to 15, w is an integer from 1 to 10, x is an integer from 2 to 15, y is an integer from 2 to 15, and z is an integer from 2 to 15.

In some embodiments, each R¹ and R² is —CH₃. In other embodiments, each R¹ and R² is —H. In still other embodiments, each R¹ and R² is independently —H or —CH₃.

In some embodiments, each X is —CH₂—, each Z is —CH—, and each R⁴ is —H.

In some embodiments, each Yⁱ is —C(O)—, each Y² is —NH— or —NCH₃—, and each [symbol] represents a single bond.

In some embodiments, the compound is

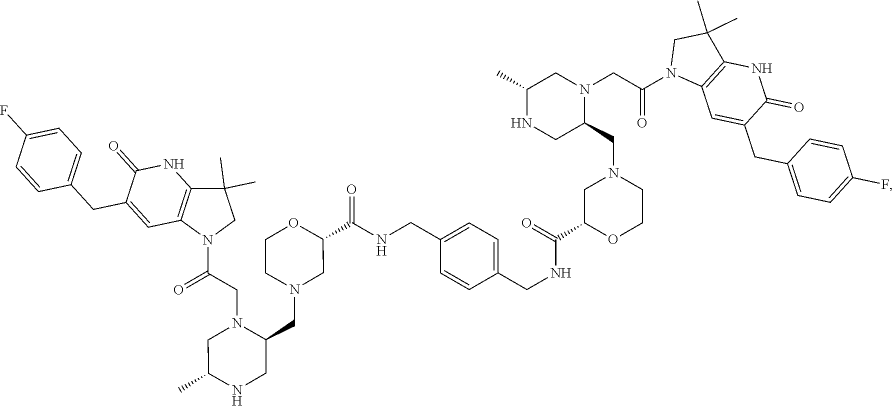 [SMILES: C[C@@H]1CN(CC(=O)N2CC(C)(C)c3[nH]c(=O)c(Cc4ccc(F)cc4)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(F)cc7)cc65)CCO4)cc3)C2)CN1]

or a pharmaceutically acceptable salt thereof.

In some embodiments, the compound is selected from the group consisting of

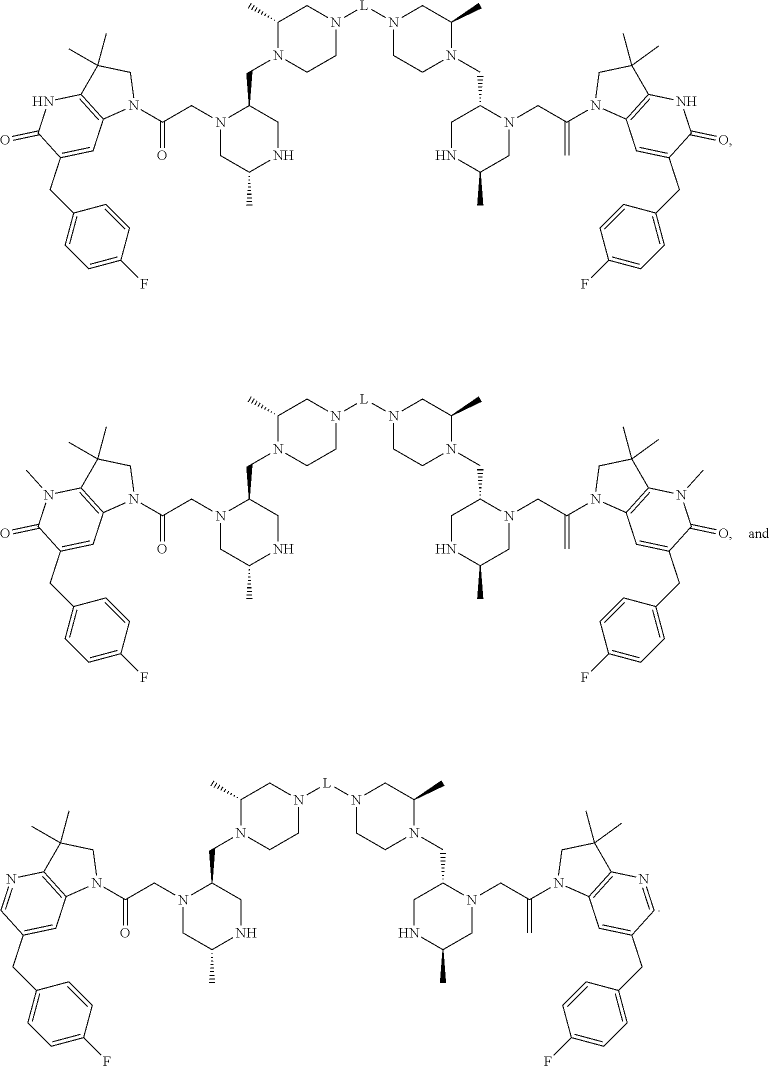

In some embodiments, L is a linker selected from the group consisting of

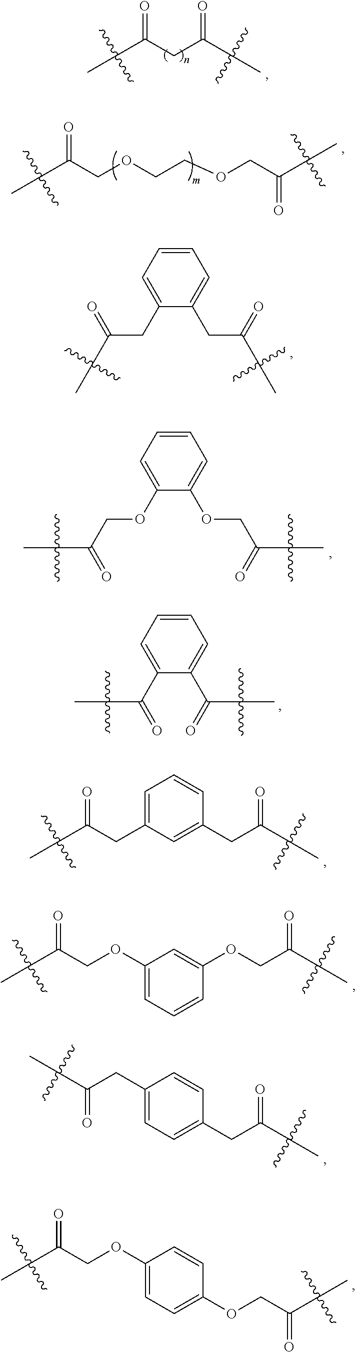

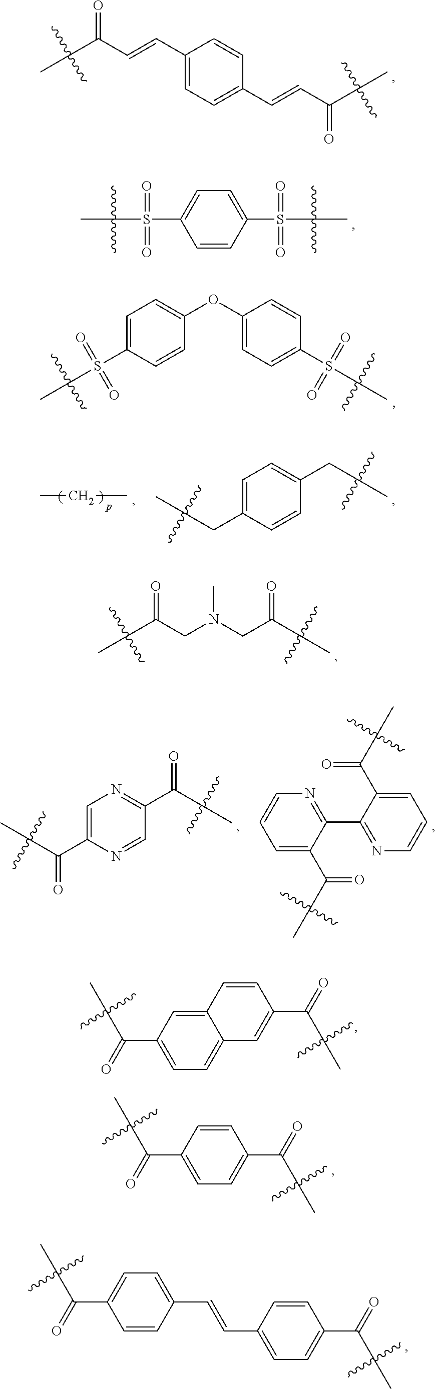

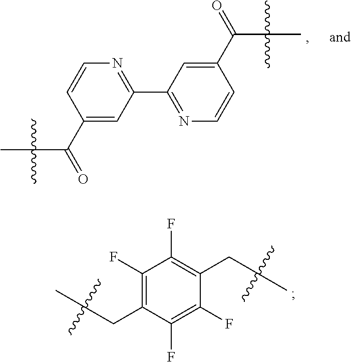 [SMILES: *C(=O)c1ccnc(-c2cc(C(*)=O)ccn2)c1 ; *Cc1c(F)c(F)c(C*)c(F)c1F]

wherein [symbol] indicates a point of attachment; and wherein n is an integer from 2 to 12, m is an integer from 1 to 3, and p is an integer from 1 to 12.

In some embodiments, the compound is

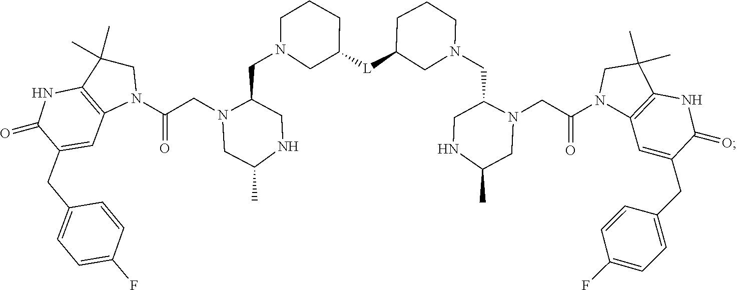

or a pharmaceutically acceptable salt or stereoisomer thereof.

In some embodiments, L is selected from the group consisting of:

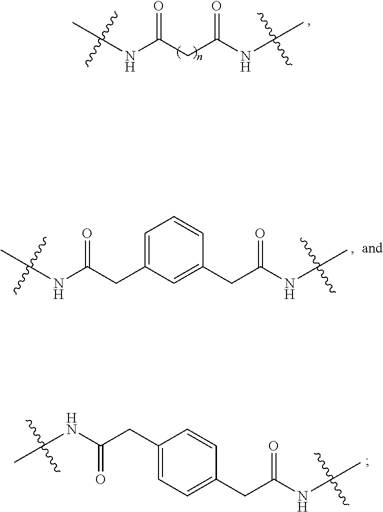

wherein [symbol] indicates a point of attachment; and wherein n is an integer from 2 to 8.

In some embodiments, the compound is selected from the group consisting of

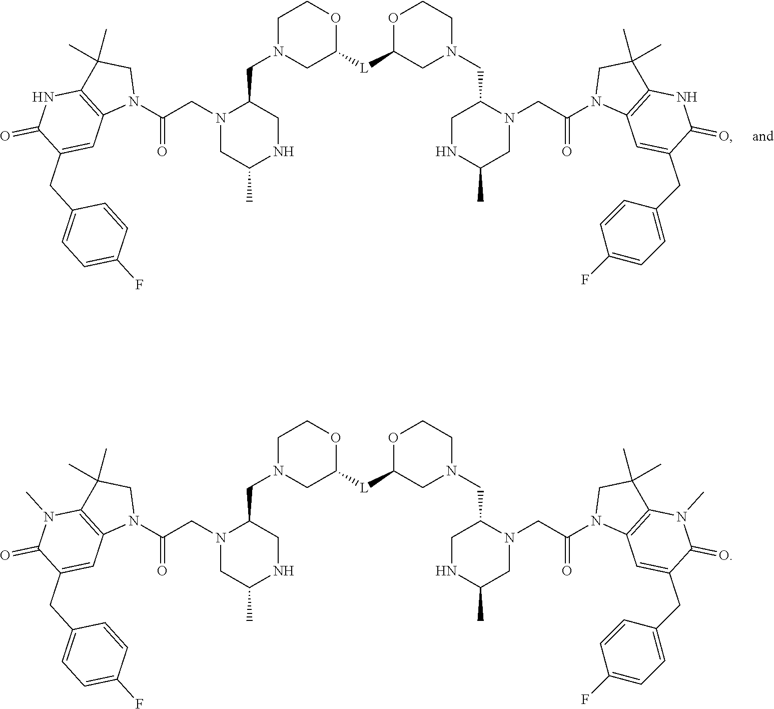

In some embodiments, L is selected from the group consisting of

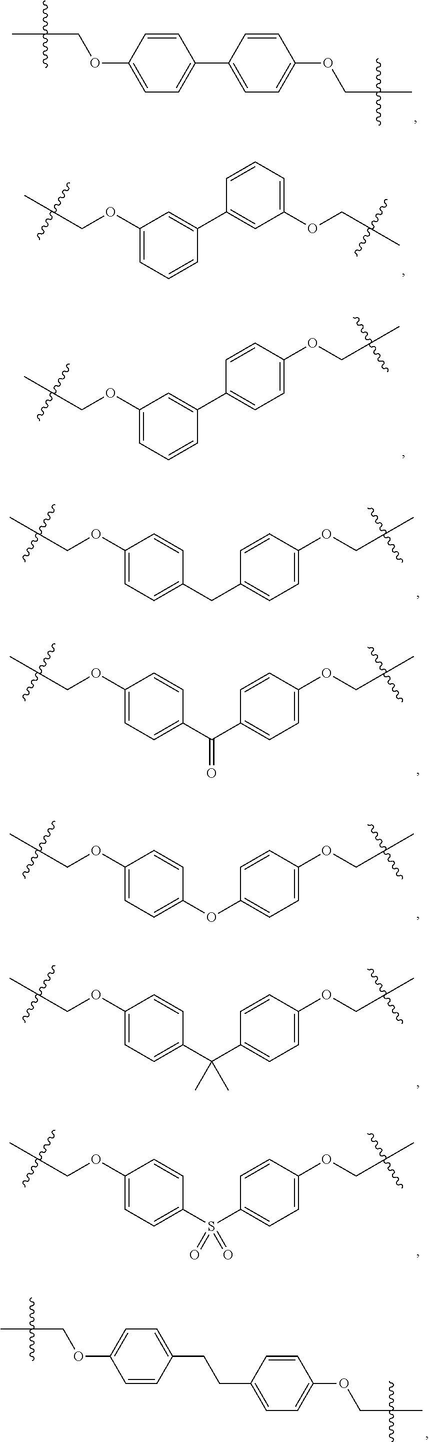

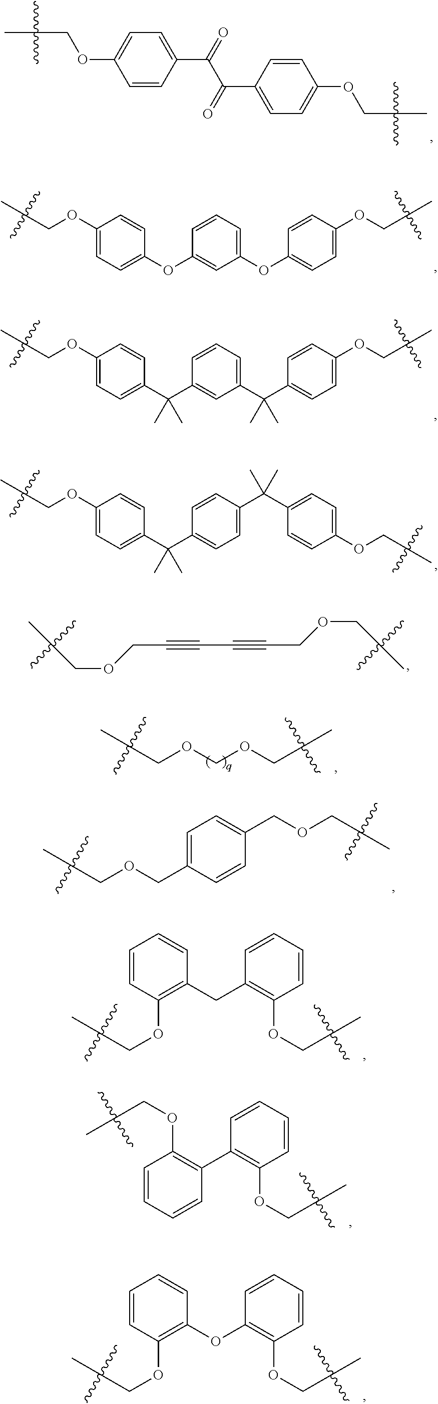

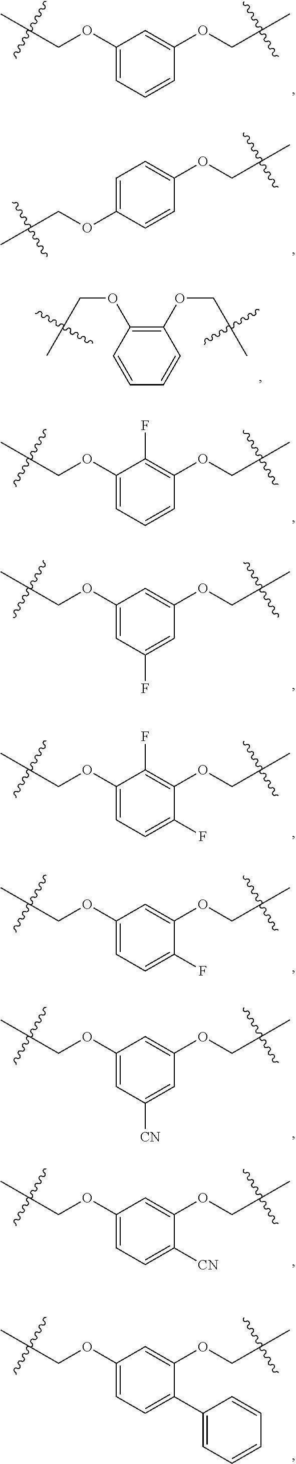

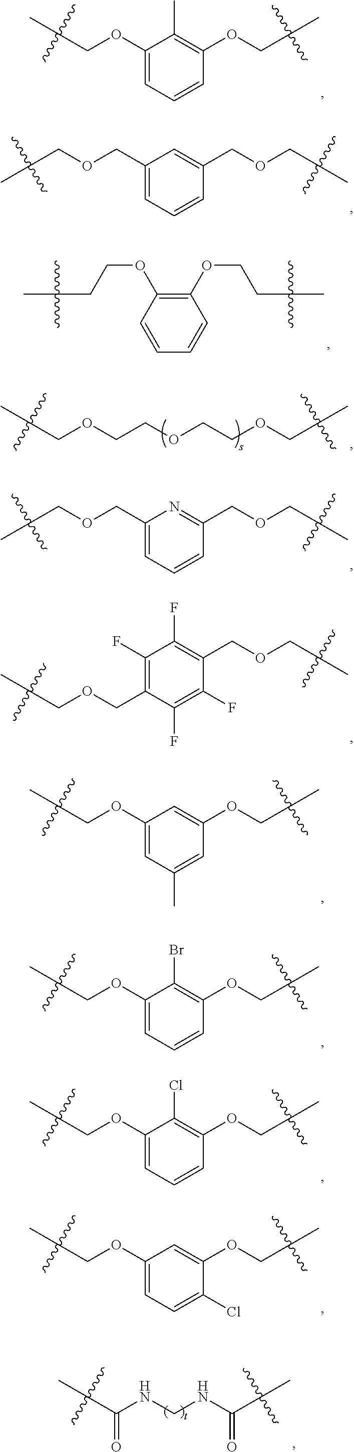

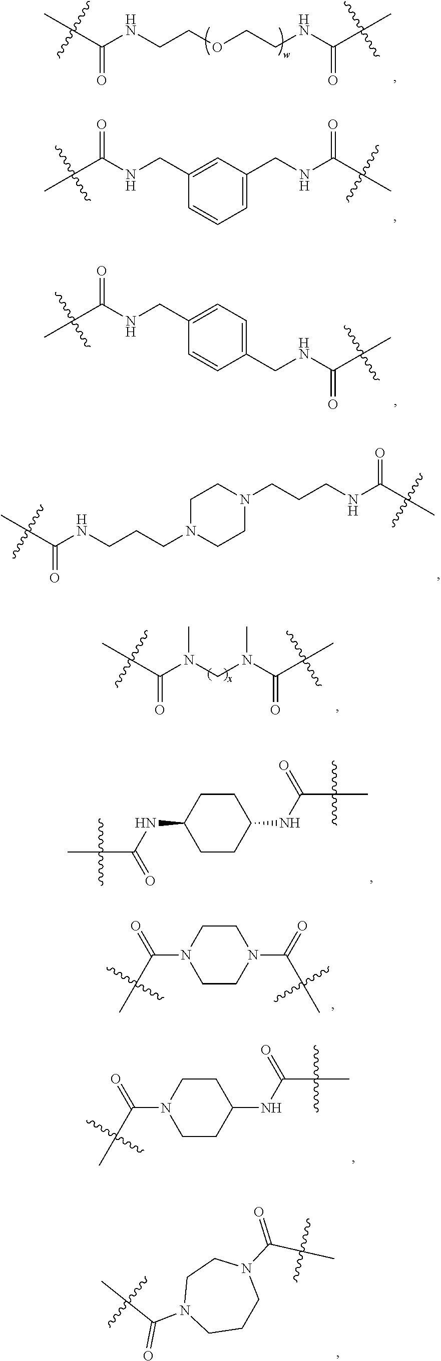

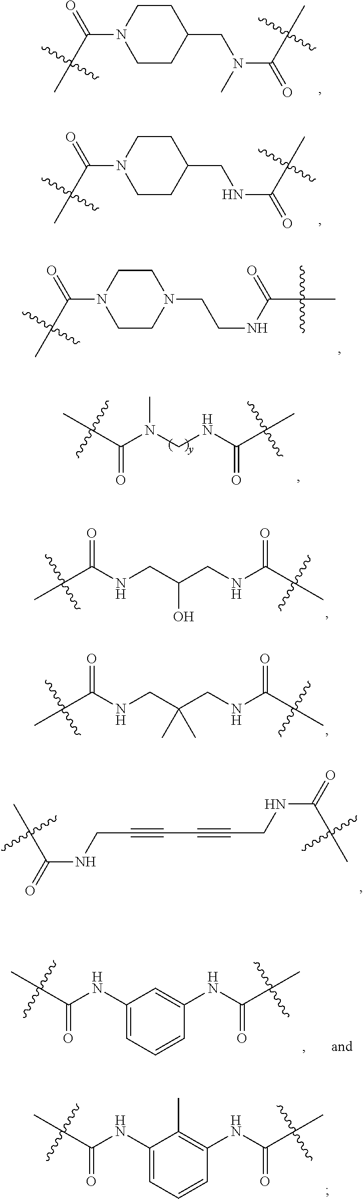

wherein “ [symbol] ” indicates a point of attachment; and wherein q is an integer from 2 to 8, s in an integer from 1 to 5, t is an integer from 2 to 8, w is an integer from 1 to 5, x is an integer from 2 to 8, and y is an integer from 2 to 8.

In some embodiments, the compound is

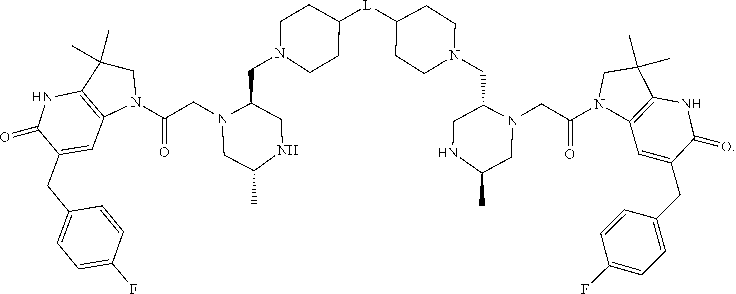

In some embodiments, L is selected from the group consisting of

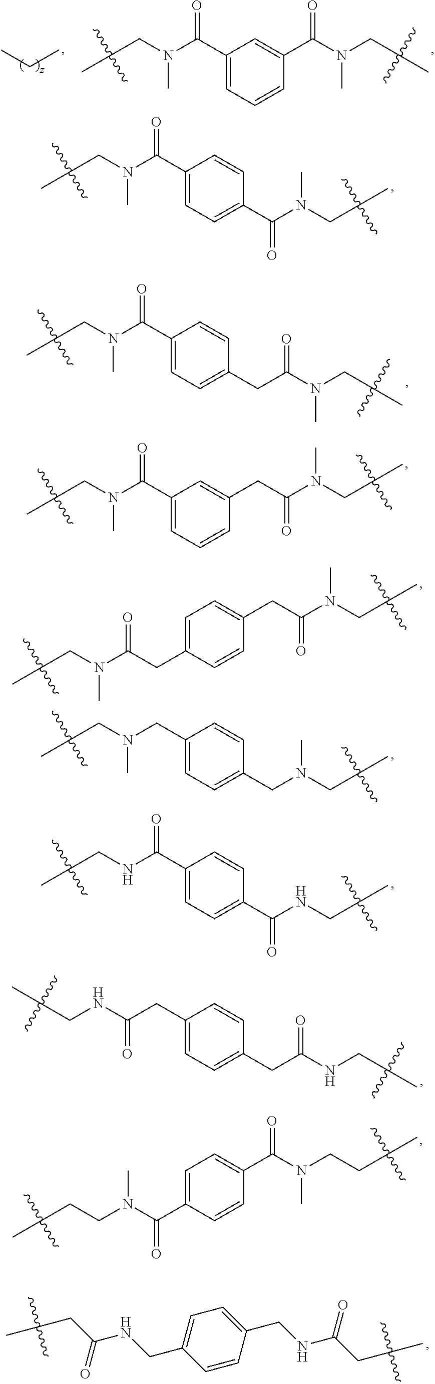

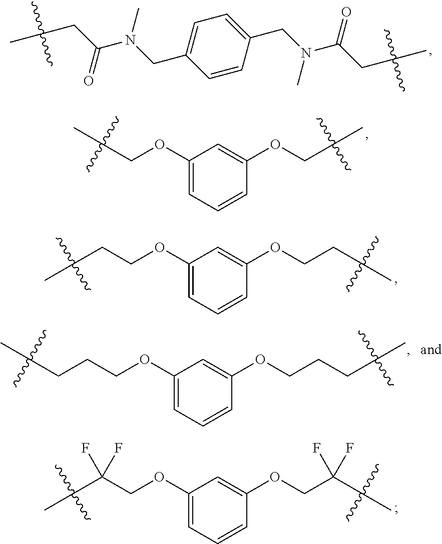

wherein “ [symbol] ” indicates a point of attachment; and wherein z is an integer from 2 to 8.

In some embodiments, the compound or pharmaceutically acceptable salt or stereoisomer thereof is a heterodimer selected from the group consisting of

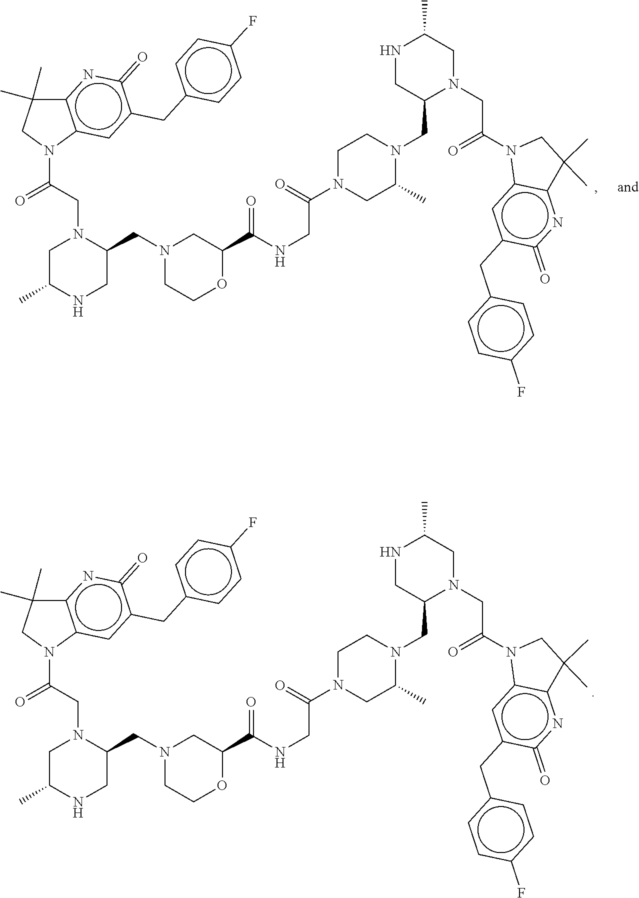

In other aspects, the invention provides a compound of the structure according to Formula (II):

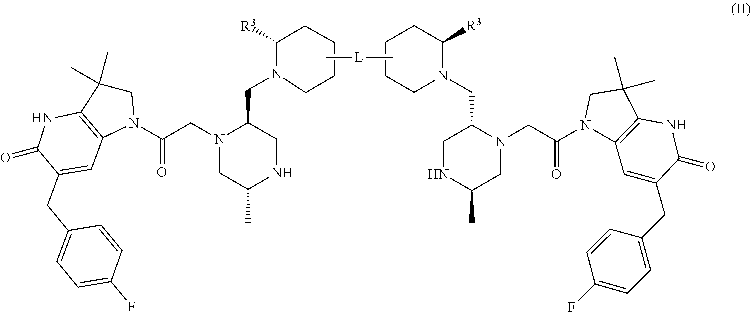

or a pharmaceutically acceptable salt or stereoisomer thereof, wherein:
each R³ is —H or —CH₃;
each W is —CH—, —O—, or —N—; and
L is a linker selected from the group consisting of

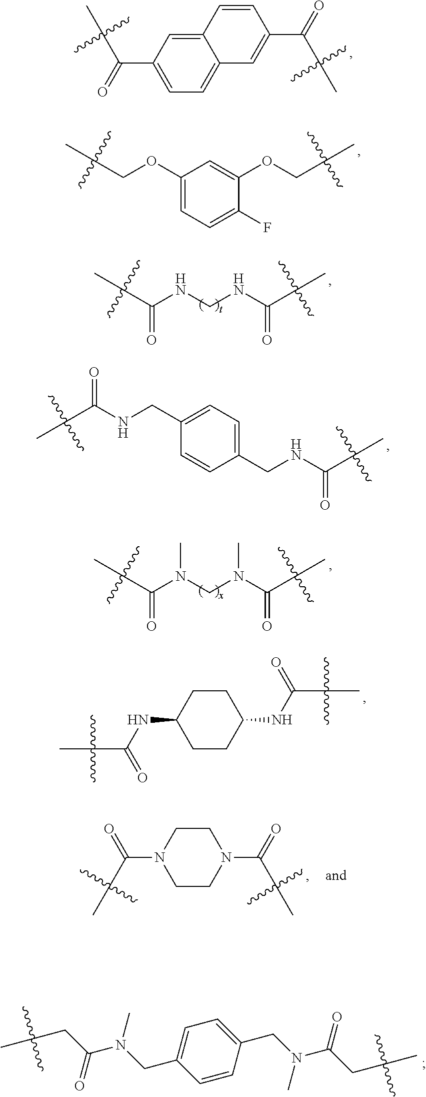

wherein “ [symbol] ” indicates a point of attachment; and wherein t is 6 or 8, and x is 3 or 6.

Exemplary compounds encompassed by the present invention include, without limitation, those in the following Table 1:

TABLE 1

| Comp | | |
|---|---|---|
| No. | Compound Structure | Compound Name |
| 1 | 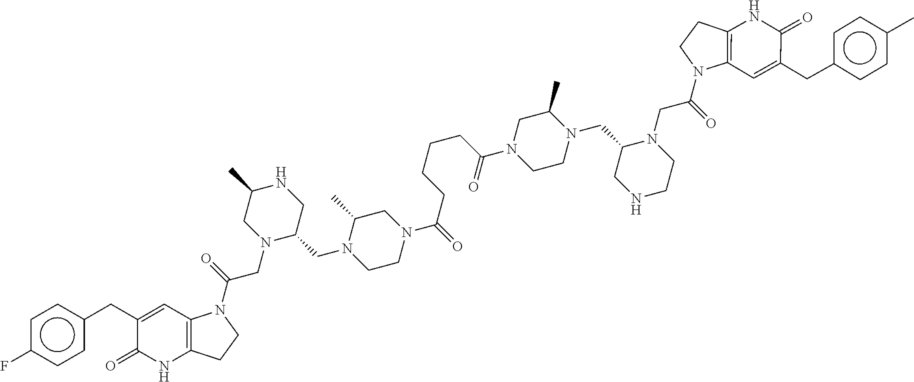 | 1,6-bis((R)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1- yl)hexane-1,6-dione |
| 2 | 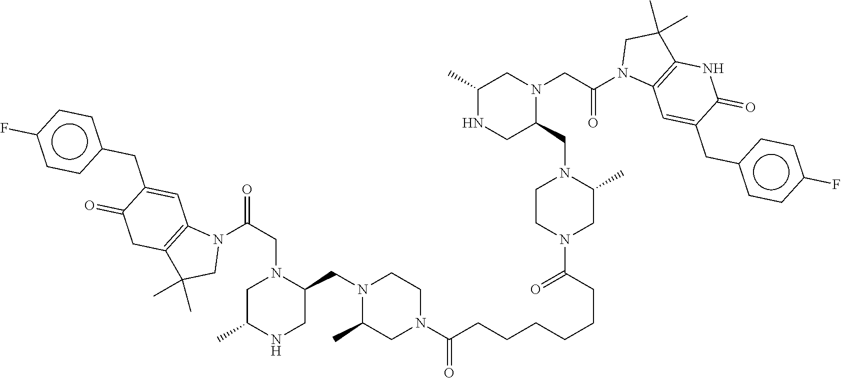 | 1,8-bis((R)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1- yl)octane-1,8-dione |
| 3 | 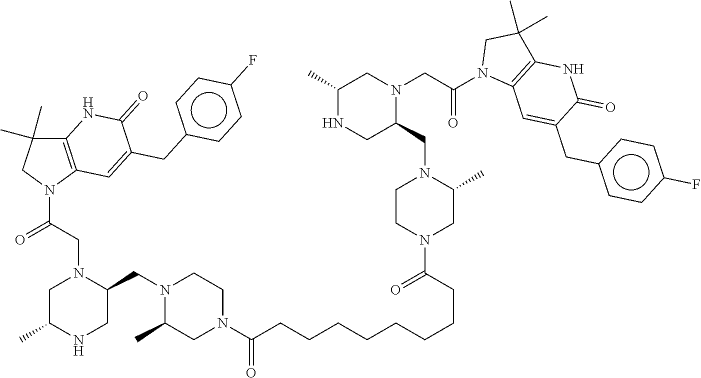 | 1,10-bis((R)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1- yl)decane-1,10-dione |
| 4 | 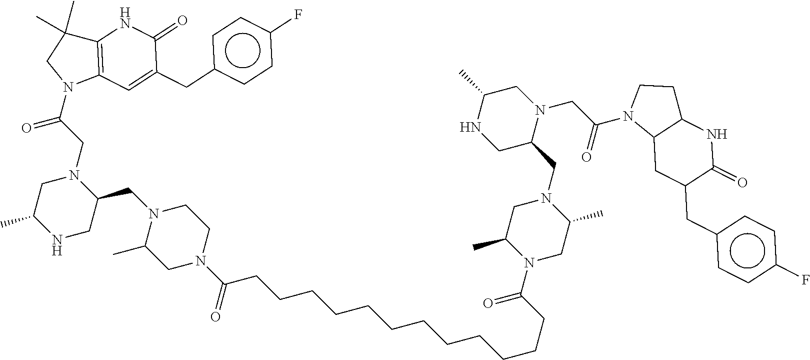 | 1,14-bis((R)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1- yl)tetradecane-1,14- dione |
| 5 | 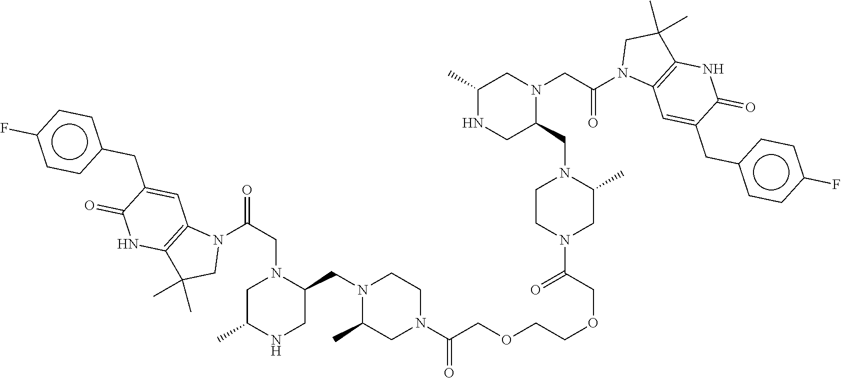 | 1,1′-(2,2′- ((3R,3′R,6R,6′R)- (((2R,2′R)-(2,2′- (ethane-1,2- diylbis(oxy))bis(acetyl)) bis(2- methylpiperazine-4,1- diyl))bis(methylene))bis (3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 6 | 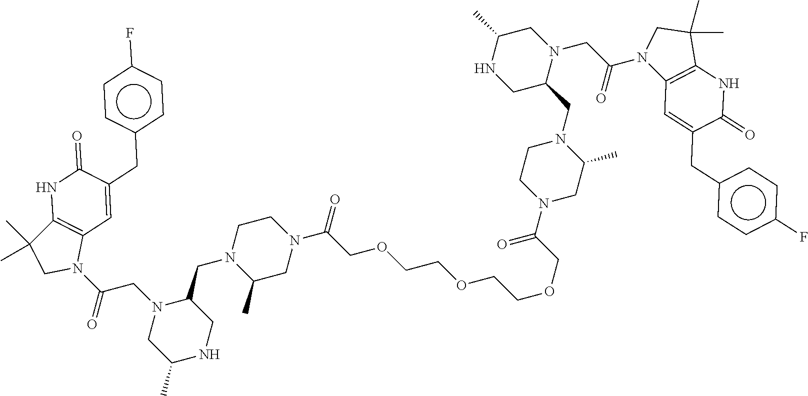 | 1,1′-(2,2′- ((3R,3′R,6R,6′R)- (((2R,2′R)-(2,2′- ((oxybis(ethane-2,1- diyl))bis(oxy))bis(acetyl)) bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 7 | 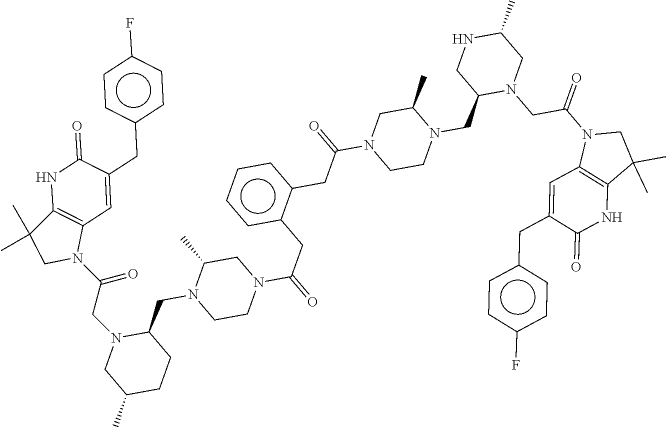 | 1,1′-(2,2′- ((3R,3′R,6R,6′R)- (((2R,2′R)-(2,2′-(1,2- phenylene)bis(acetyl)) bis(2-methylpiperazine- 4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 8 | 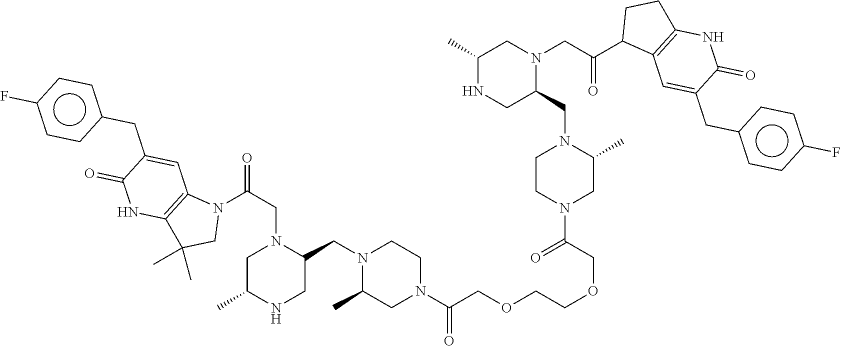 | 1,1′-(2,2′- ((3R,3′R,6R,6′R)- (((2R,2′R)-(2,2′-(1,2- phenylenebis(oxy))bis (acetyl))bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 9 | 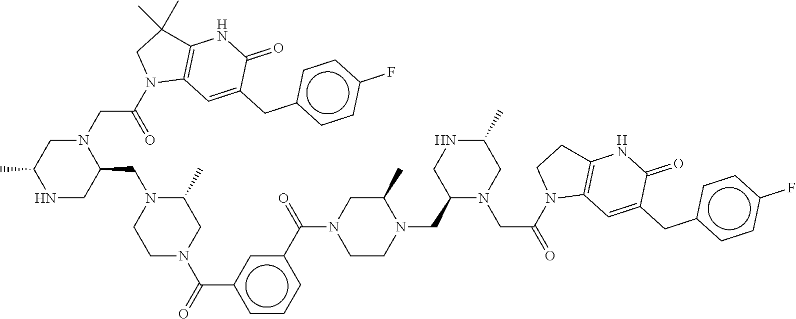 | 1,1′-(2,2′- ((3R,3′R,6R,6′R)- (((2R,2′R)- isophthaloylbis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 10 | 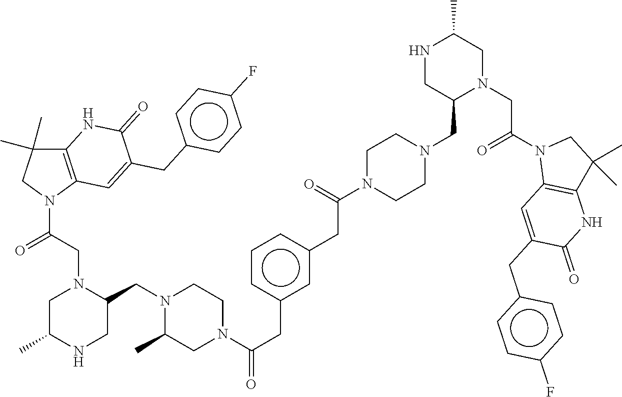 | 1,1′-(2,2′- ((3R,3′R,6R,6′R)- (((2R,2′R)-(2,2′-(1,3- phenylene)bis(acetyl)) bis(2-methylpiperazine- 4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 11 | 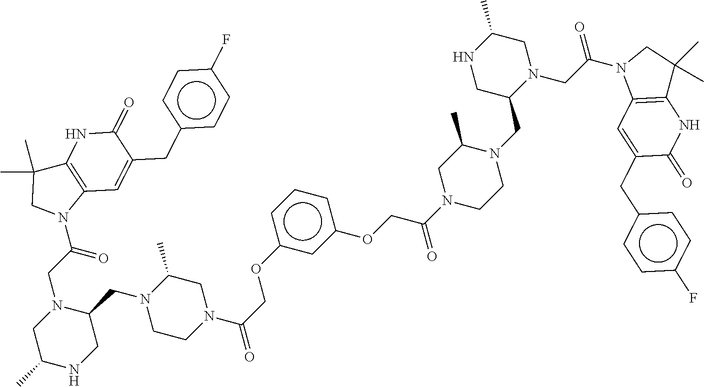 | 1,1′-(2,2′- ((3R,3′R,6R,6′R)- (((2R,2′R)-(2,2′-(1,3- phenylenebis(oxy))bis (acetyl))bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5HI- pyrrolo[3,2-b]pyridin- 5-one) |
| 12 | 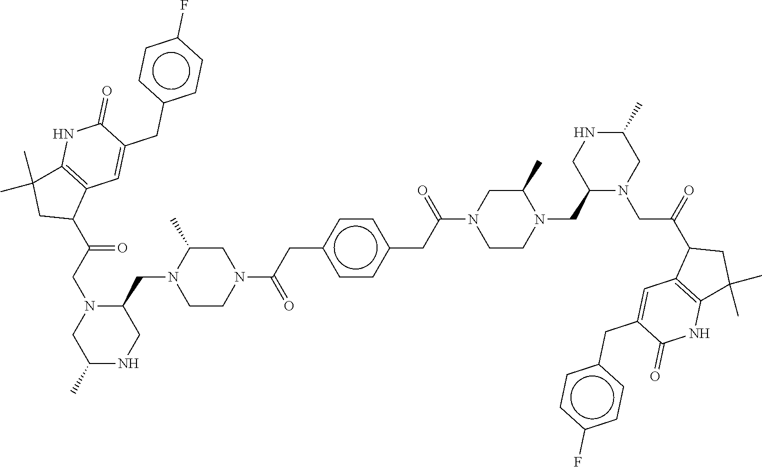 | 1,1′-(2,2′- ((3R,3′R,6R,6′R)- (((2R,2′R)-(2,2′-(1,4- phenylene)bis(acetyl)) bis(2-methylpiperazine- 4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 13 | 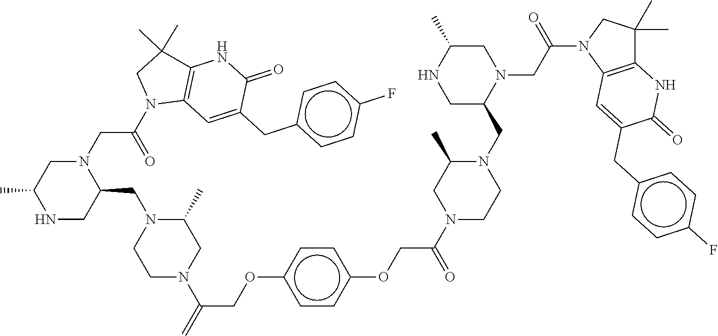 | 1,1′-(2,2′- ((3R,3′R,6R,6′R)- (((2R,2′R)-(2,2′-(1,4- phenylenebis(oxy))bis (acetyl))bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5HI- pyrrolo[3,2-b]pyridin- 5-one) |
| 14 | 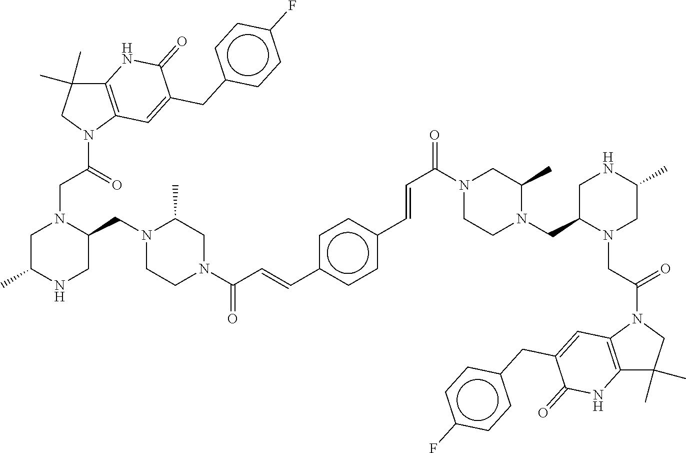 | 1,1′-(2,2′- ((3R,3′R,6R,6′R)- (((2R,2′R)-((2E,2′E)- 3,3′-(1,4- phenylene)bis(acryloyl)) bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 15 | 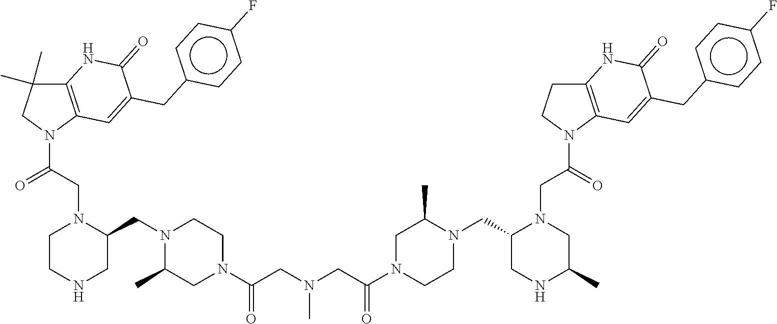 | 1,1′-(2,2′- ((3R,3′R,6R,6′R)- (((2R,2′R)-(2,2′- (methylazanediyl)bis (acetyl))bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 16 | 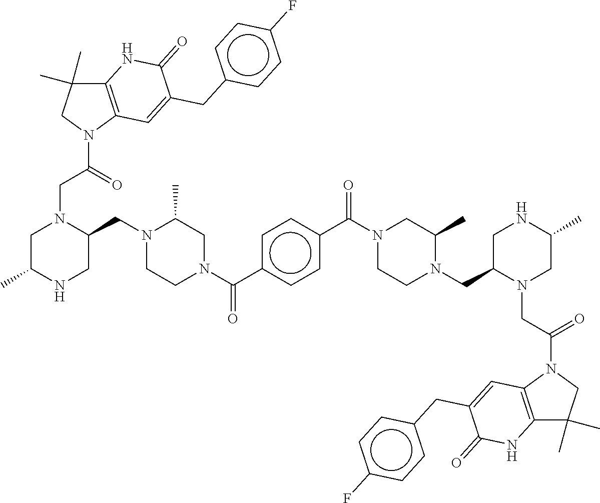 | 1,1′-(2,2′- ((3R,3′R,6R,6′R)- (((2R,2′R)-(pyrazine- 2,5-dicarbonyl)bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 17 | 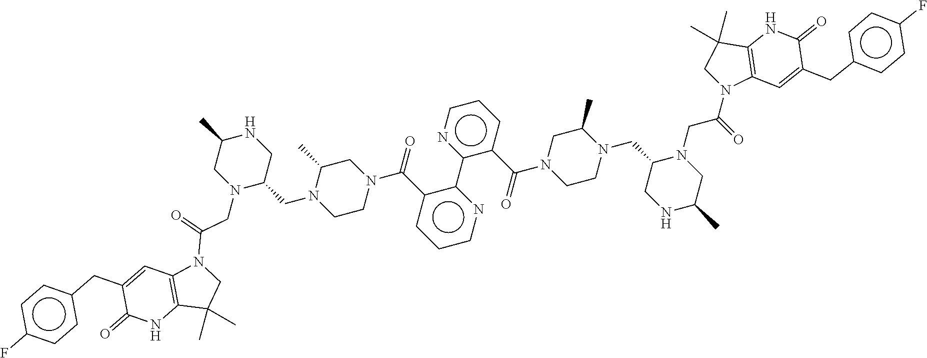 | 1,1′-(2,2′- ((3R,3′R,6R,6′R)- (((2R,2′R)-([2,2′- bipyridine]-3,3′- dicarbonyl)bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 18 | 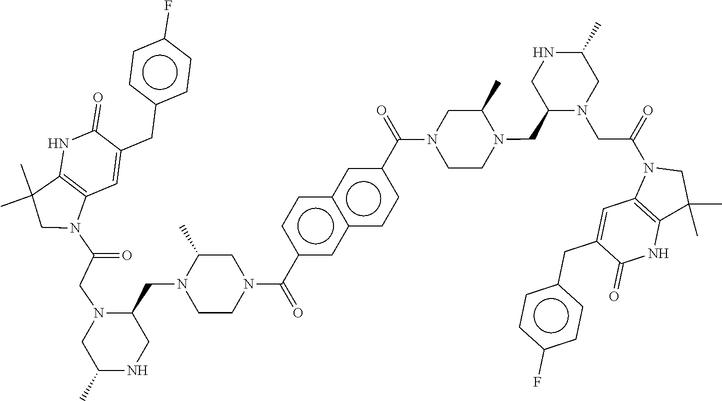 | 1,1′-(2,2′- ((3R,3′R,6R,6′R)- (((2R,2′R)- (naphthalene-2,6- dicarbonyl)bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 19 | 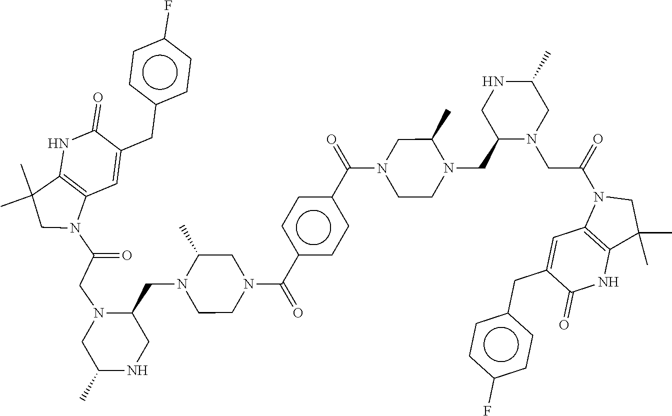 | 1,1′-(2,2′- ((3R,3′R,6R,6′R)- (((2R,2′R)- terephthaloylbis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 20 | 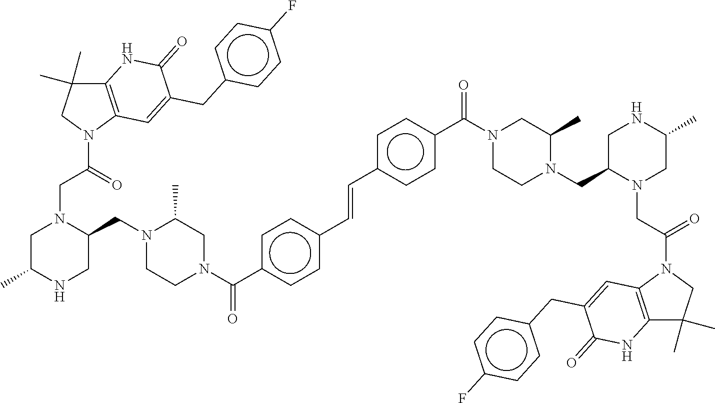 | 1,1′-(2,2′- ((3R,3′R,6R,6′R)- (((2R,2′R)-(4,4′-((E)- ethene-1,2- diyl)bis(benzoyl))bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 21 | 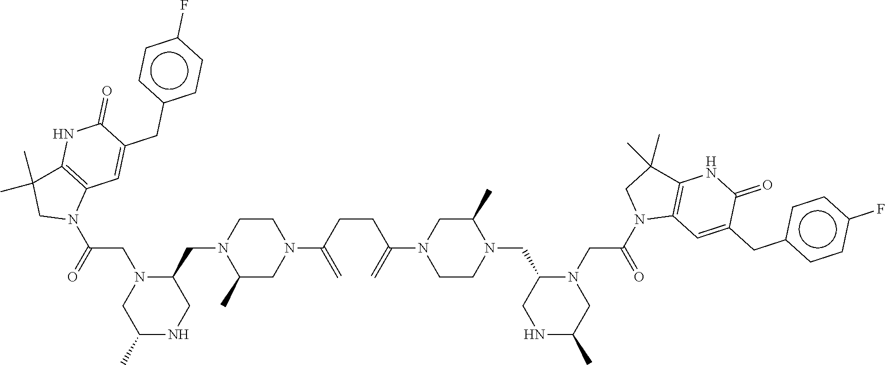 | 1,4-bis((R)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1- yl)butane-1,4-dione |
| 22 | 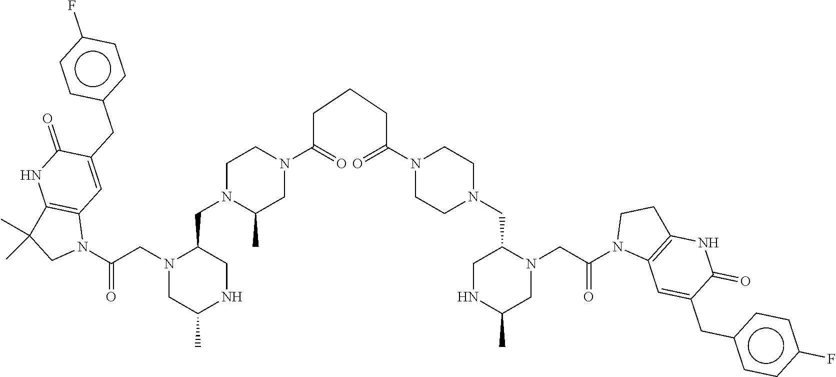 | 1,5-bis((R)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1- yl)pentane-1,5-dione |
| 23 | 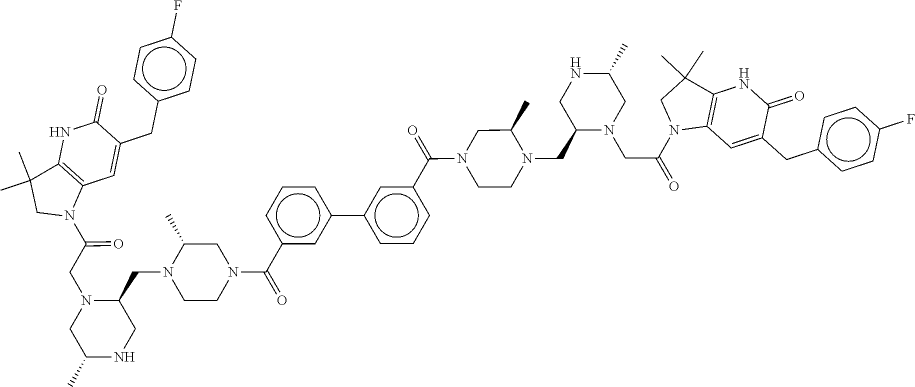 | 1,1′-(2,2′- ((3R,3′R,6R,6′R)- (((2R,2′R)-([2,2′- bipyridine]-4,4′- dicarbonyl)bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 24 | 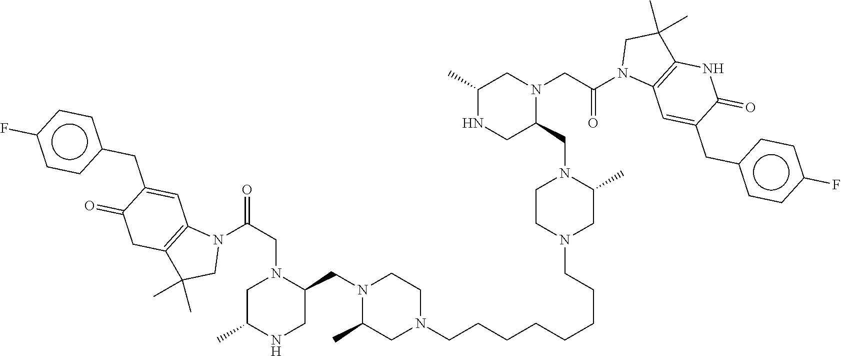 | 1,1′-(2,2′- ((3R,3′R,6R,6′R)- (((2R,2′R)-octane-1,8- diylbis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 25 | 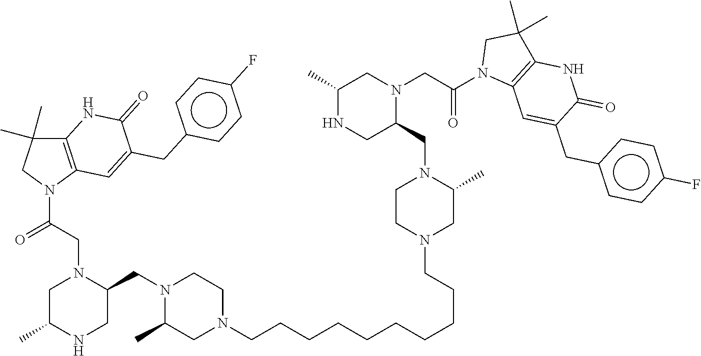 | 1,1′-(2,2′- ((3R,3′R,6R,6′R)- (((2R,2′R)-decane-1,10- diylbis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 26 | 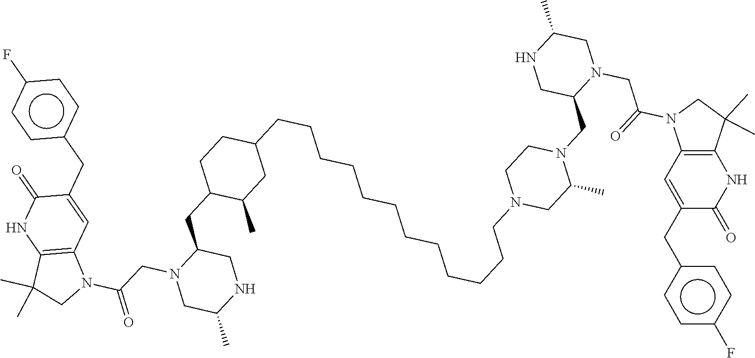 | 1,1′-(2,2′- ((3R,3′R,6R,6′R)- (((2R,2′R)-dodecane- 1,12-diylbis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 27 | 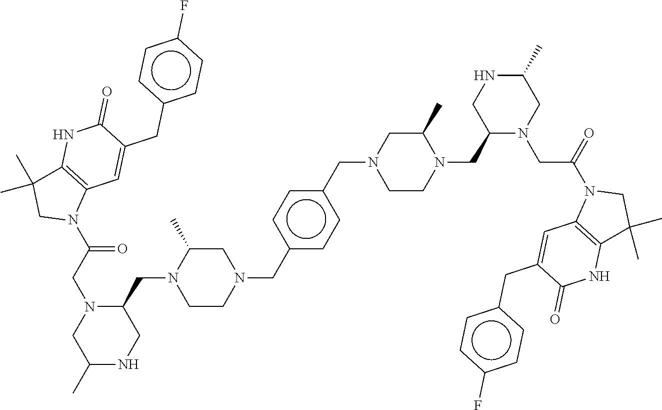 | 1,1′-(2,2′- ((3R,3′R,6R,6′R)- (((2R,2′R)-(1,4- phenylenebis(methylene)) bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 28 | 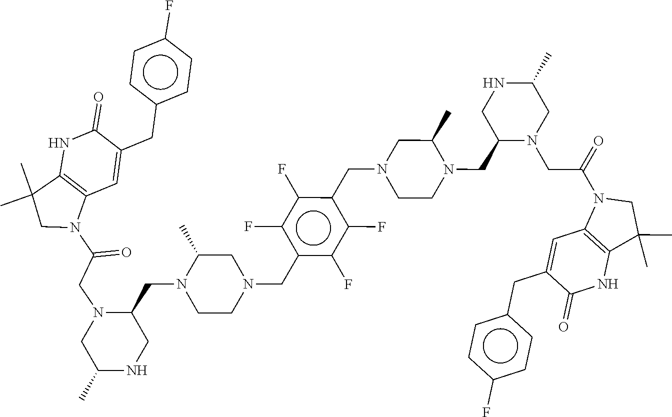 | 1,1′-(2,2′- ((3R,3′R,6R,6′R)- (((2R,2′R)-((perfluoro- 1,4- phenylene)bis(methylene)) bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 29 | 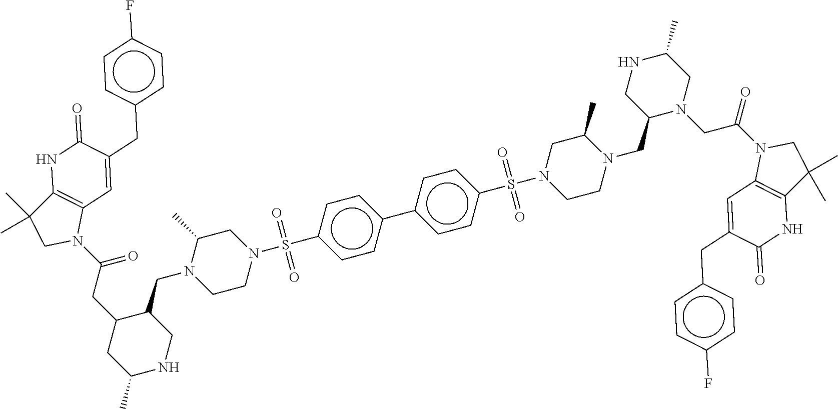 | 1,1′-(2,2′- ((3R,3′R,6R,6′R)- (((2R,2′R)-([1,1′- biphenyl]-4,4′- disulfonyl)bis(2- methylpiperazine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 30 | 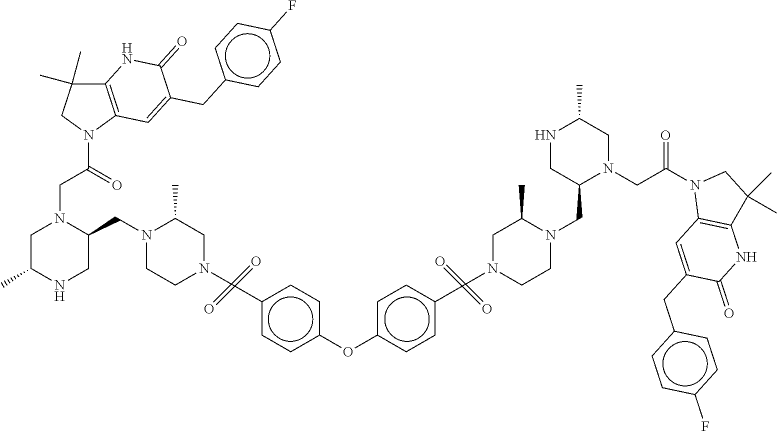 | 2,2′-((3R,3′R,6R,6′R)- (((2R,2′R)-(oxybis(4,1- phenylenesulfonyl))bis (2-methylpiperazine- 4,1- diyl))bis(methylene)) bis(3-methylpiperazine- 6,1-diyl))bis(1-(6-(4- fluorobenzyl)-3,3- dimethyl-5-oxo- 2,3,4,5-tetrahydro-1H- pyrrolo[3,2-b]pyridin- 1-yl)ethan-1-one) |
| 31 | 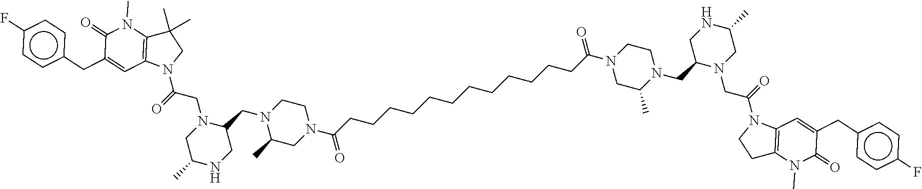 | 1,14-bis((R)-4- (((2R,5R)-1-(2-(6-(4- fluorobenzyl)-3,3,4- trimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1- yl)tetradecane-1,14- dione |
| 32 | 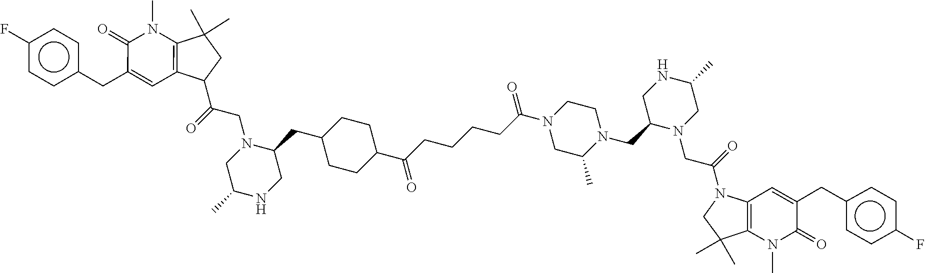 | 1,6-bis((R)-4- (((2R,5R)-1-(2-(6-(4- fluorobenzyl)-3,3,4- trimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1- yl)hexane-1,6-dione |
| 33 | 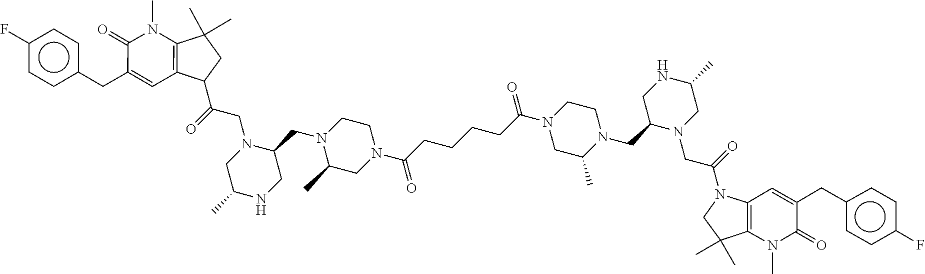 | 1,8-bis((R)-4- (((2R,5R)-1-(2-(6-(4- fluorobenzyl)-3,3,4- trimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1- yl)octane-1,8-dione |
| 34 | 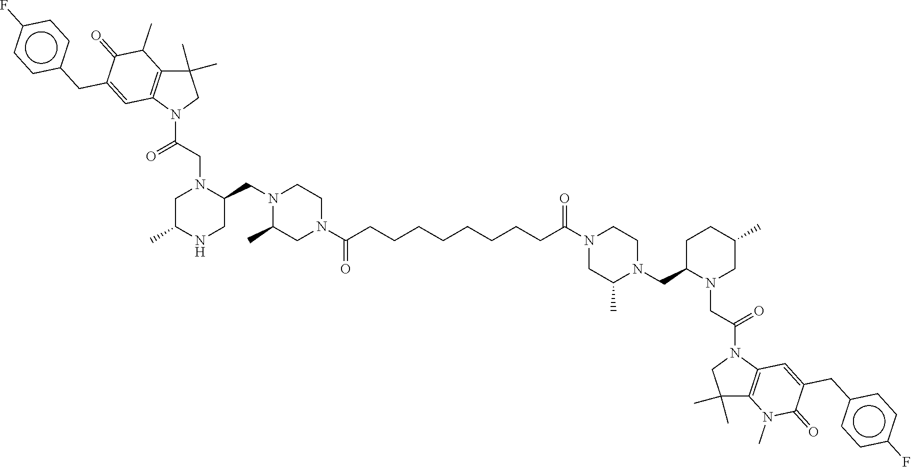 | 1,10-bis((R)-4- (((2R,5R)-1-(2-(6-(4- fluorobenzyl)-3,3,4- trimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1- yl)decane-1,10-dione |
| 35 | 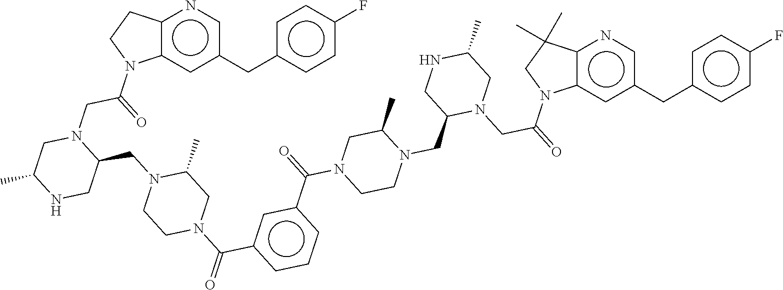 | 2,2′-((3R,3′R,6R,6′R)- (((2R,2′R)- isophthaloylbis(2- methylpiperazine-4,1- diyl))bis(methylene)) bis(3-methylpiperazine- 6,1-diyl))bis(1-(6-(4- fluorobenzyl)-3,3- dimethyl-2,3-dihydro- 1H-pyrrolo[3,2- b]pyridin-1-yl)ethan-1- one) |
| 36 | 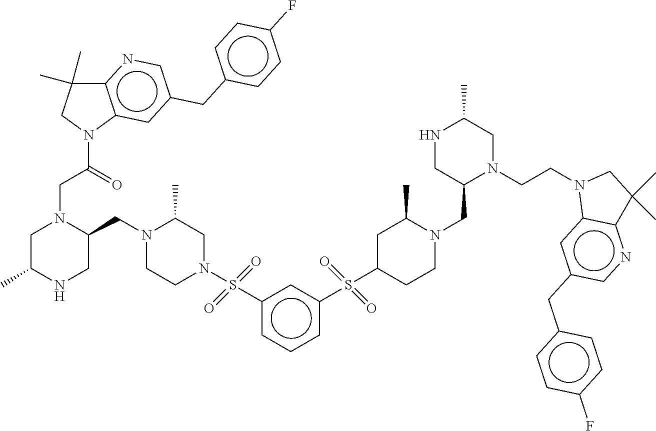 | 2,2′-((3R,3′R,6R,6′R)- (((2R,2′R)-(1,3- phenylenedisulfonyl) bis(2-methylpiperazine- 4,1- diyl))bis(methylene)) bis(3-methylpiperazine- 6,1-diyl))bis(1-(6-(4- fluorobenzyl)-3,3- dimethyl-2,3-dihydro- 1H-pyrrolo[3,2- b]pyridin-1-yl)ethan-1- one) |
| 37 | 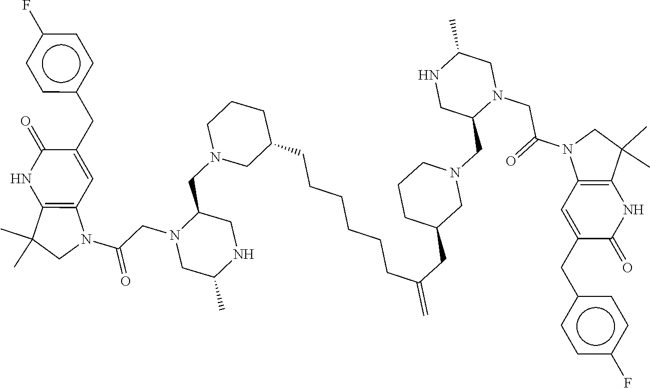 | N1,N6-bis((S)-1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)adipamide |
| 38 | 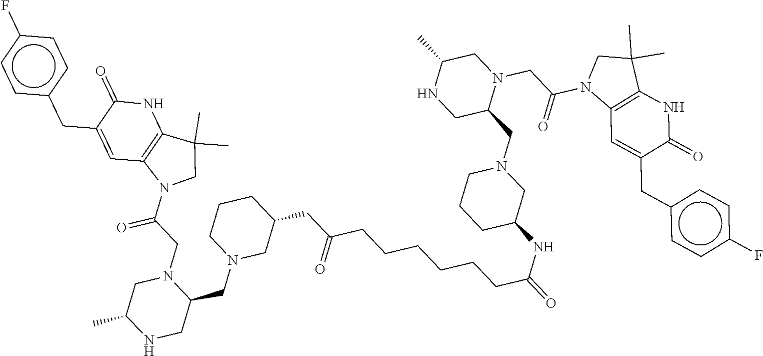 | N1,N8-bis((S)-1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)octanediamide |
| 39 | 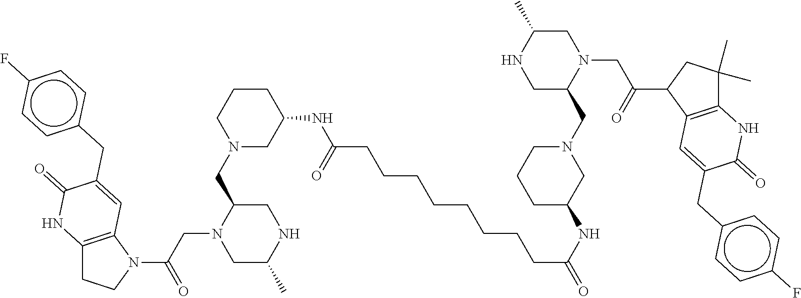 | N1,N10-bis((S)-1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)decanediamide |
| 40 | 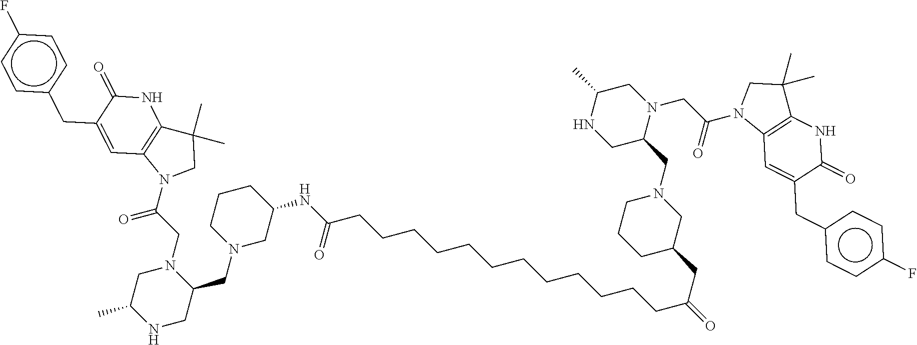 | N1,N14-bis((S)-1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)tetradecanediamide |
| 41 | 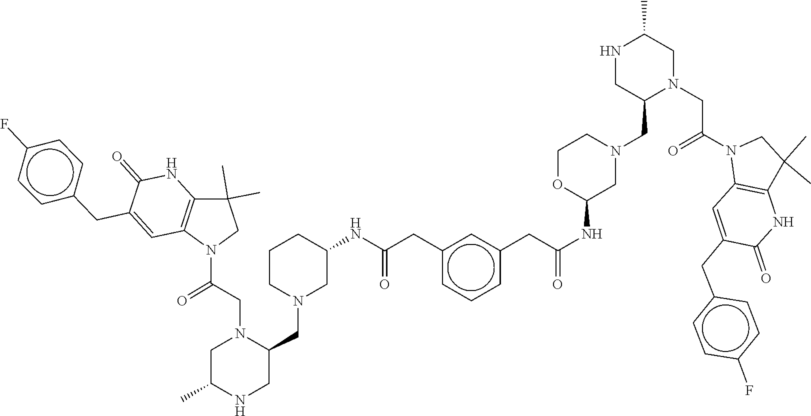 | 2,2′-(1,3- phenylene)bis(N-((S)- 1-(((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)acetamide) |
| 42 | 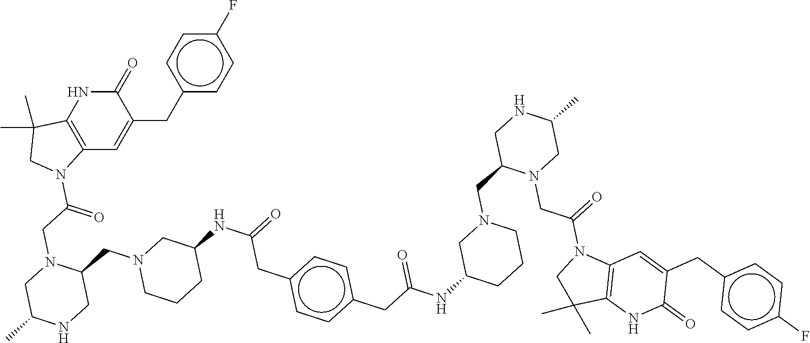 | 2,2′-(1,4- phenylene)bis(N-((S)- 1-(((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)acetamide) |
| 43 | 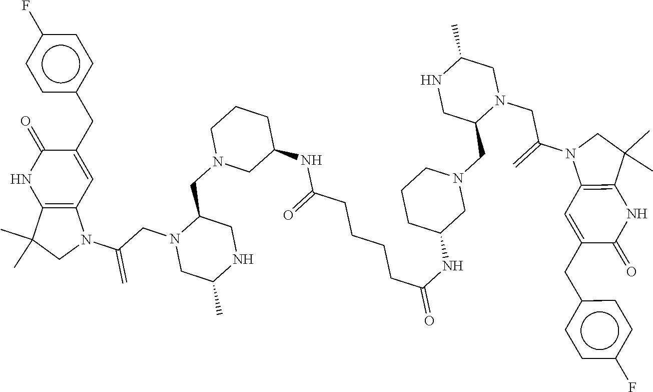 | N1,N6-bis((R)-1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)adipamide |
| 44 | 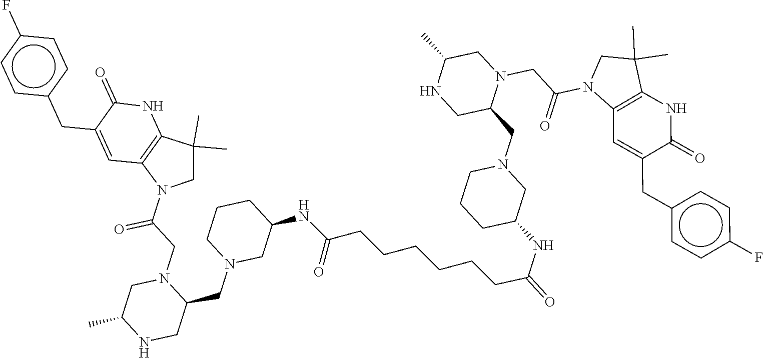 | N1,N8-bis((R)-1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)octanediamide |
| 45 | 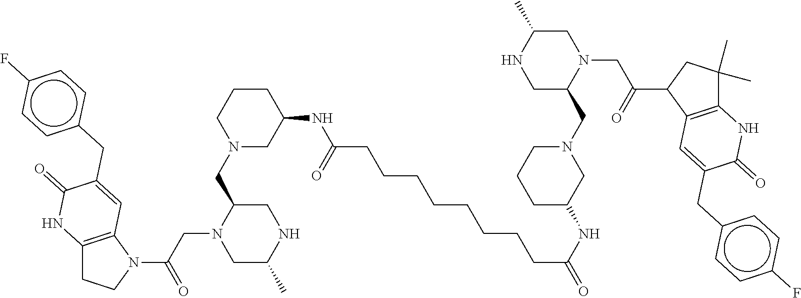 | N1,N10-bis((R)-1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)decanediamide |
| 46 | 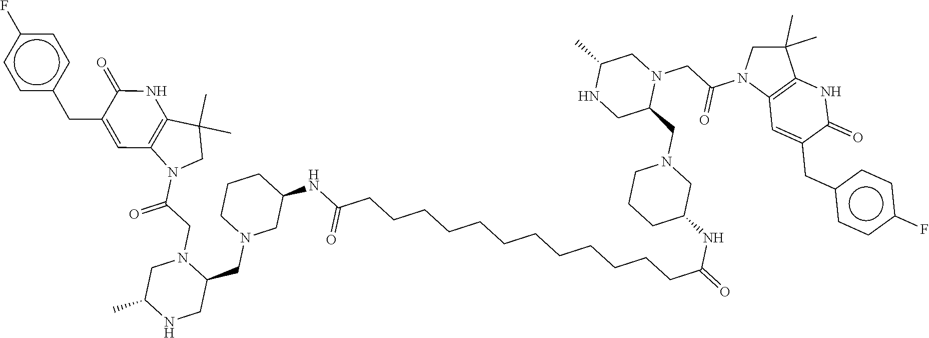 | N1,N14-bis((R)-1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)tetradecanediamide |
| 47 | 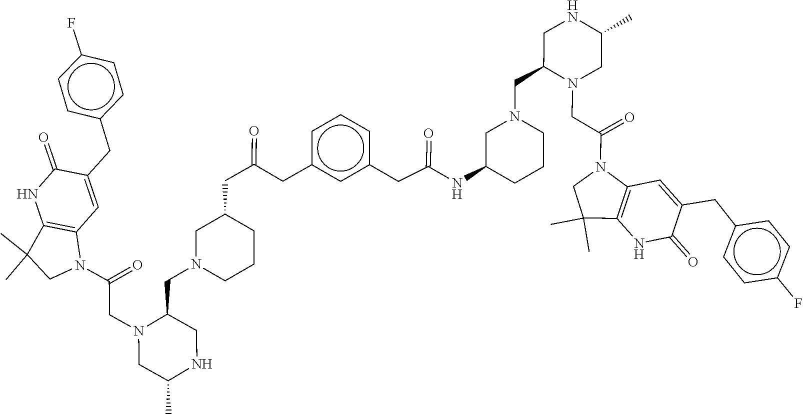 | 2,2′-(1,3- phenylene)bis(N-((R)- 1-(((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)acetamide) |
| 48 | 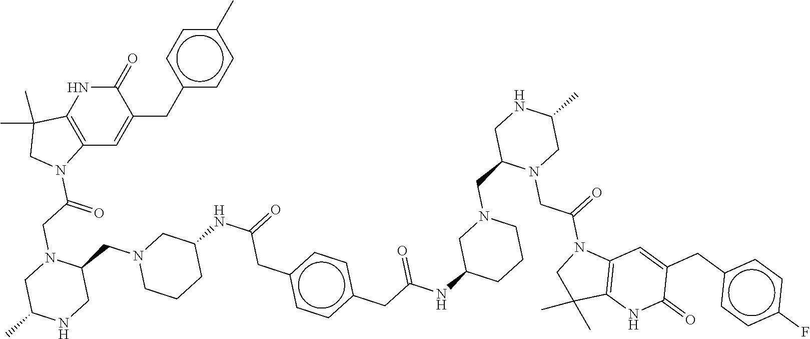 | 2,2′-(1,4- phenylene)bis(N-((R)- 1-(((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-3- yl)acetamide) |
| 49 | 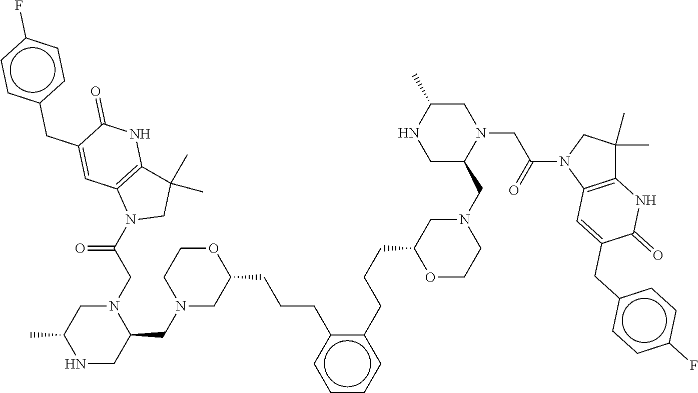 | 1,1′-(2,2′- ((3R,3′R,6R,6′R)- (((2S,2′S)-(((1,2- phenylenebis(methylene)) bis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 50 | 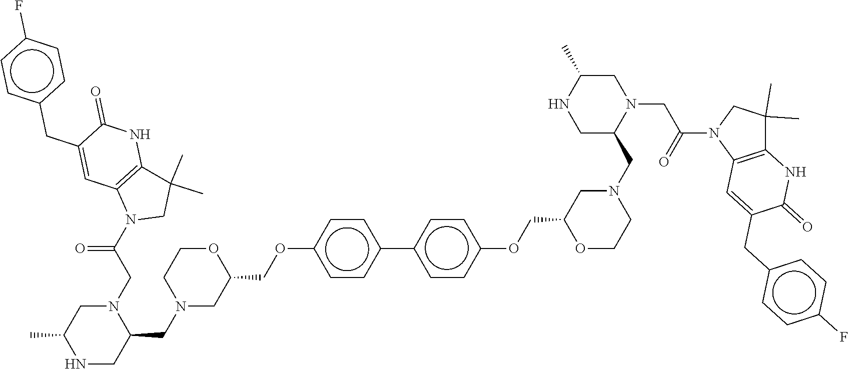 | 1,1′-(2,2′- ((3R,3′R,6R,6′R)- (((2S,2′S)-(([1,1′- biphenyl]-4,4′- diylbis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 51 | 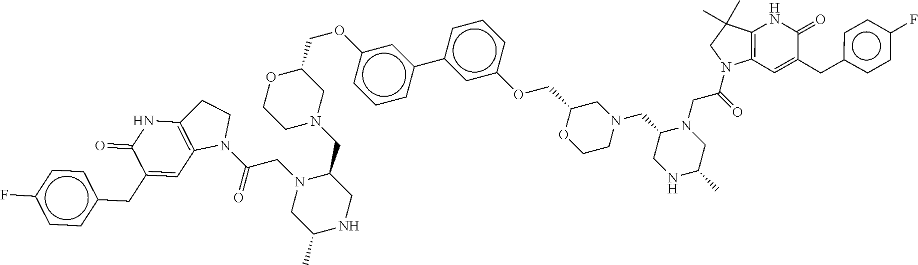 | 1,1′-(2,2′- ((3R,3′R,6R,6′R)- (((2S,2′S)-(([1,1′- biphenyl]-3,3′- diylbis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 52 | 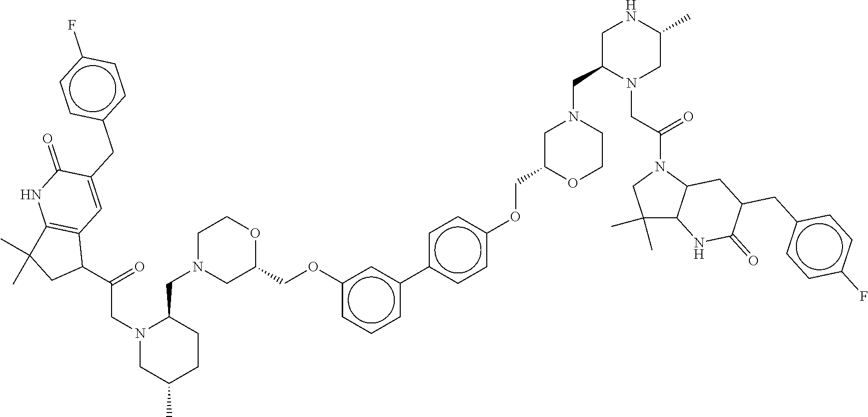 | 1,1′-(2,2′- ((3R,3′R,6R,6′R)- (((2S,2′S)-(([1,1′- biphenyl]-3,4′- diylbis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 53 | 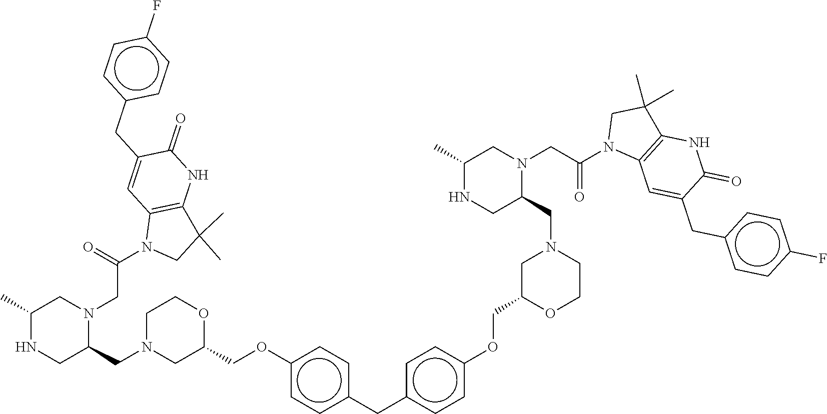 | 1,1′-(2,2′- ((3R,3′R,6R,6′R)- (((2S,2′S)- (((methylenebis(4,1- phenylene))bis(oxy)) bis(methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 54 | 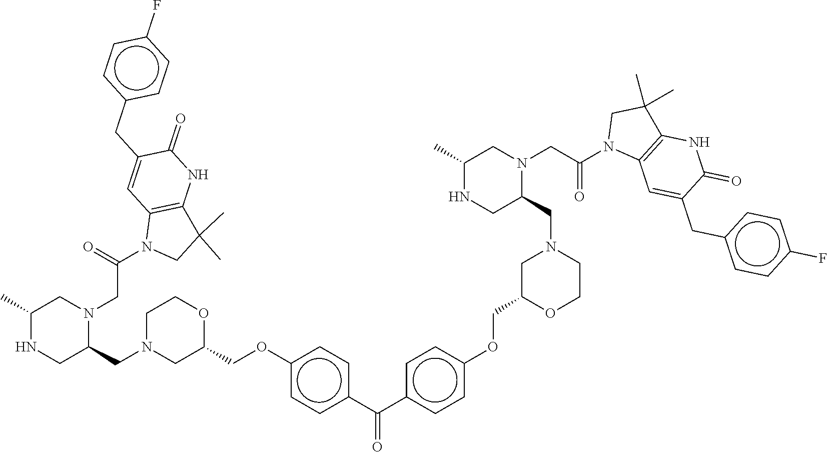 | 1,1′-(2,2′- ((3R,3′R,6R,6′R)- (((2S,2′S)- (((carbonylbis(4,1- phenylene))bis(oxy)) bis(methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 55 | 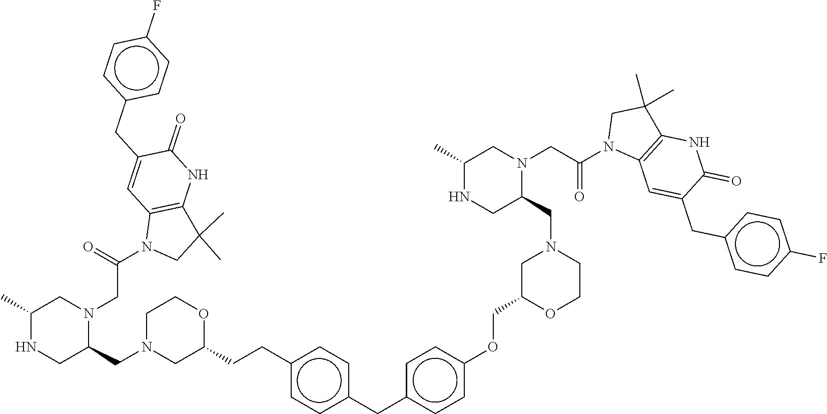 | 1,1′-(2,2′- ((3R,3′R,6R,6′R)- (((2S,2′S)- (((oxybis(4,1- phenylene))bis(oxy)) bis(methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 56 | 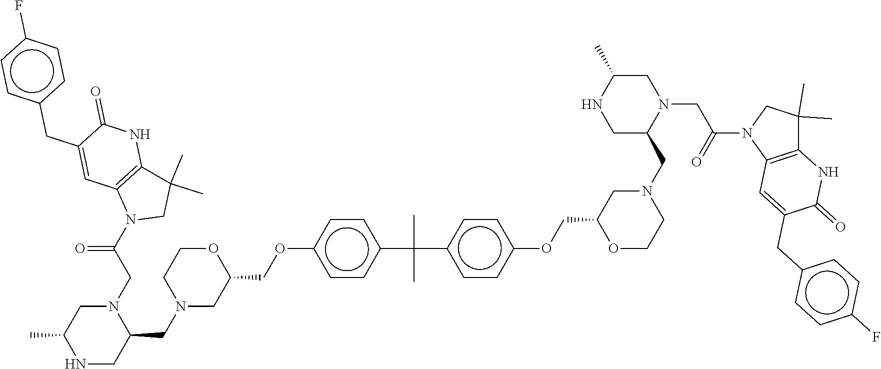 | 1,1′-(2,2′- ((3R,3′R,6R,6′R)- (((2S,2′S)-(((propane- 2,2-diylbis(4,1- phenylene))bis(oxy)) bis(methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5/- pyrrolo[3,2-b]pyridin- 5-one) |
| 57 | 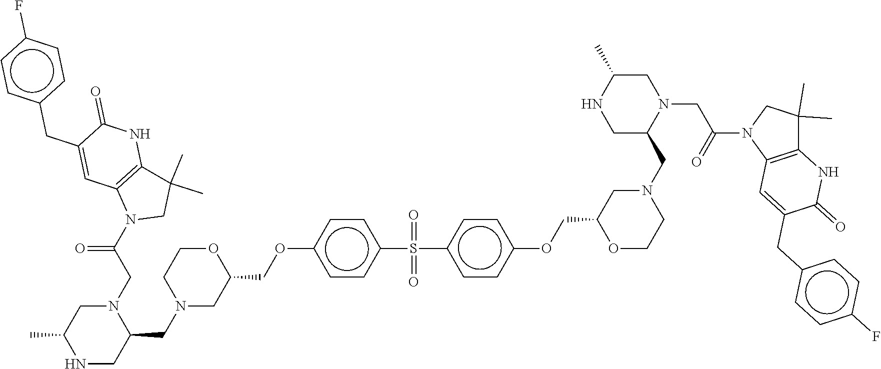 | 1,1′-(2,2′- ((3R,3′R,6R,6′R)- (((2S,2′S)- (((sulfonylbis(4,1- phenylene))bis(oxy)) bis(methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 58 | 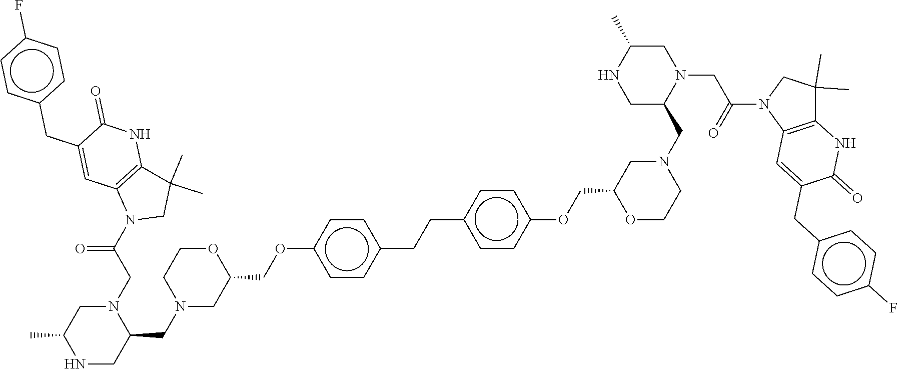 | 1,1′-(2,2′- ((3R,3′R,6R,6′R)- (((2S,2′S)-(((ethane- 1,2-diylbis(4,1- phenylene))bis(oxy)) bis(methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 59 | 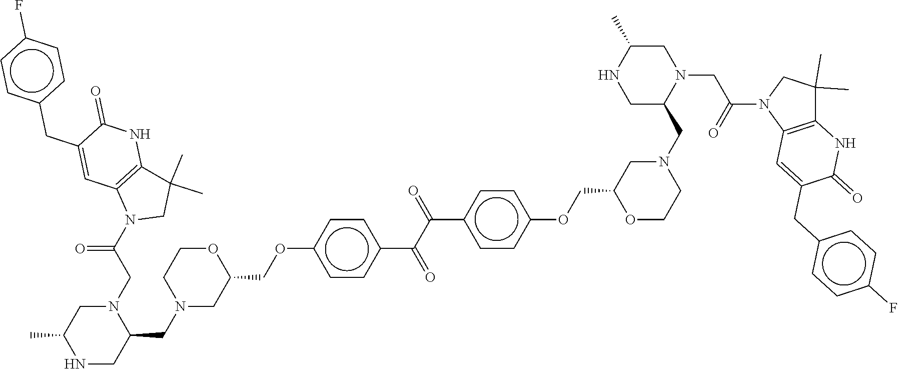 | 1,2-bis(4-(((S)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholin-2- yl)methoxy)phenyl)eth ane-1,2-dione |
| 60 | 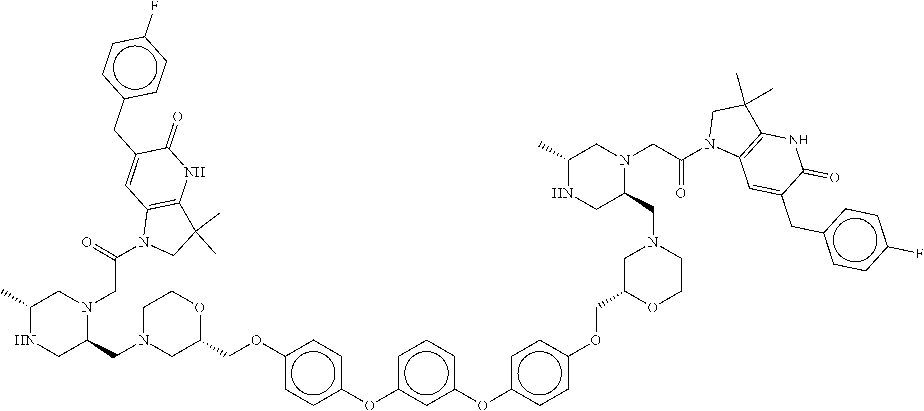 | 1,1′-(2,2′- ((3R,3′R,6R,6′R)- (((2S,2′S)-((((1,3- phenylenebis(oxy))bis (4,1- phenylene))bis(oxy)) bis(methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 61 | 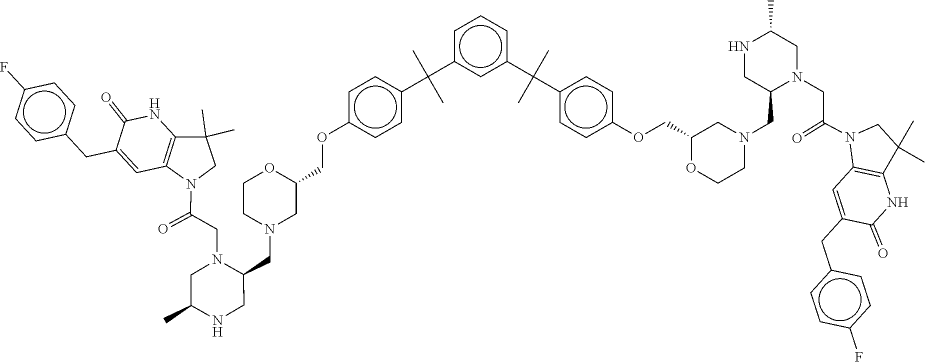 | 1,1′-(2,2′- ((3R,3′R,6R,6′R)- (((2S,2′S)-((((1,3- phenylenebis(propane- 2,2-diyl))bis(4,1- phenylene))bis(oxy)) bis(methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 62 | 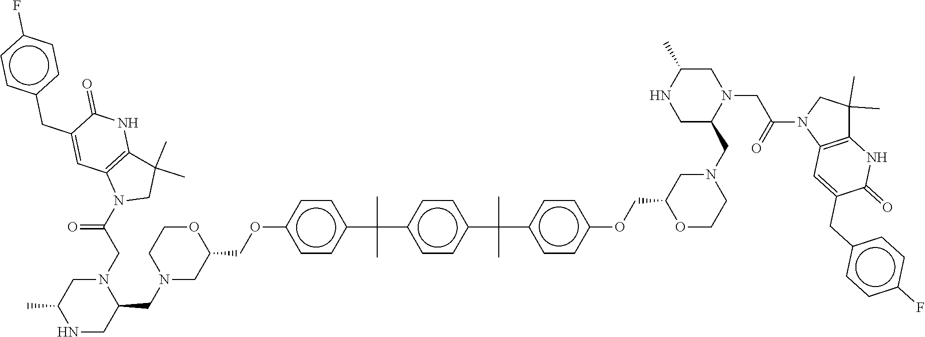 | 1,1′-(2,2′- ((3R,3′R,6R,6′R)- (((2S,2′S)-((((1,4- phenylenebis(propane- 2,2-diyl))bis(4,1- phenylene))bis(oxy)) bis(methylene))bis (morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 63 | 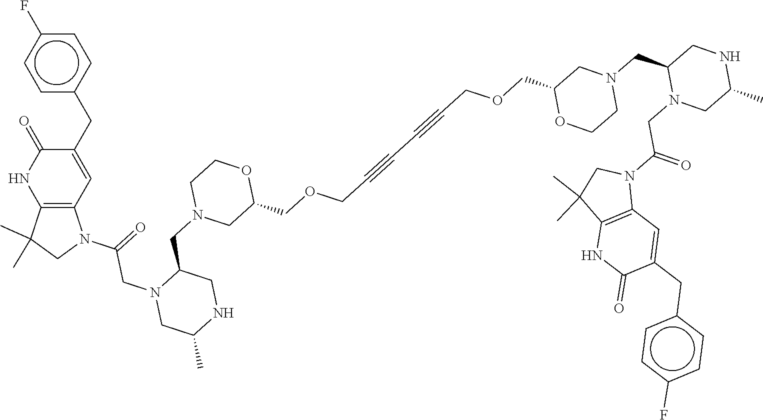 | 1,1′-(2,2′- ((3R,3′R,6R,6′R)- (((2S,2′S)-((hexa-2,4- diyne-1,6- diylbis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 64 | 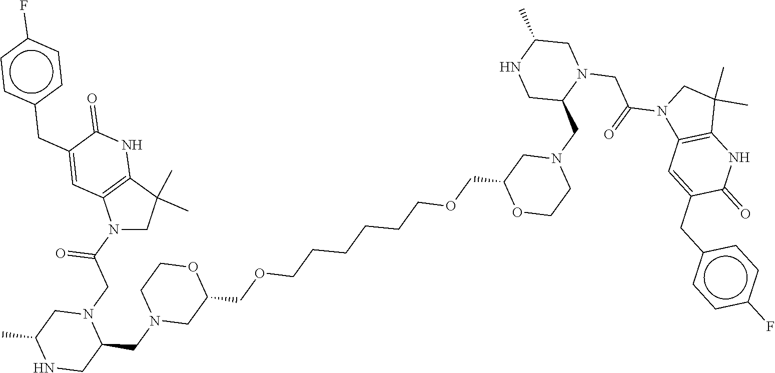 | 1,1′-(2,2′- ((3R,3′R,6R,6′R)- (((2S,2′S)-((hexane-1,6- diylbis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 65 | 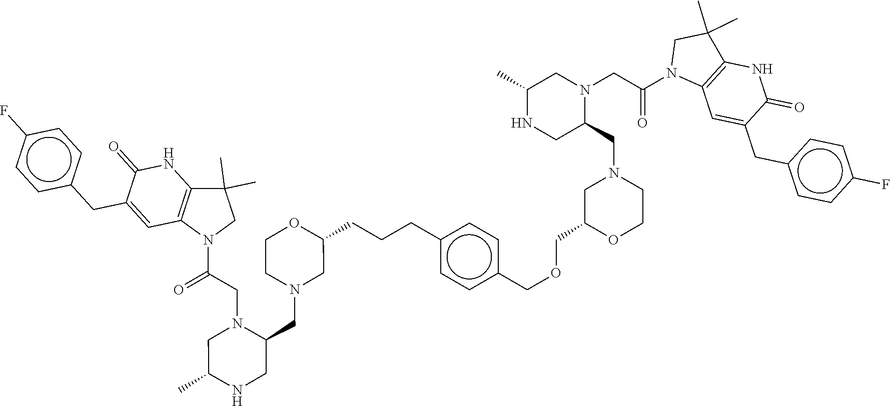 | 1,1′-(2,2′- ((3R,3′R,6R,6′R)- (((2S,2′S)-(((1,4- phenylenebis(methylene)) bis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 66 | 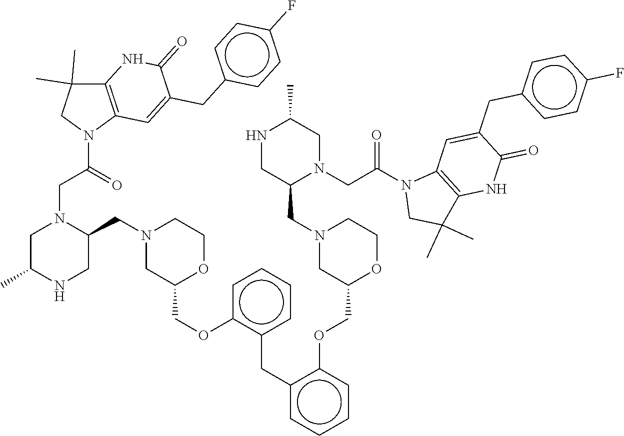 | 1,1′-(2,2′- ((3R,3′R,6R,6′R)- (((2S,2′S)- (((methylenebis(2,1- phenylene))bis(oxy)) bis(methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 67 | 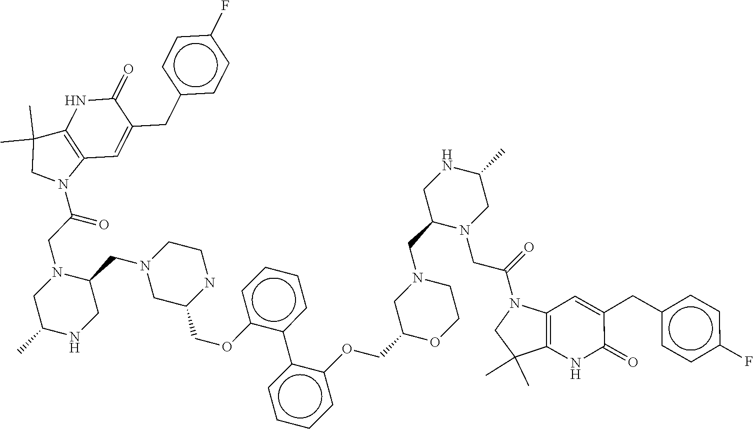 | 1,1′-(2,2′- ((3R,3′R,6R,6′R)- (((2S,2′S)-(([1,1′- biphenyl]-2,2′- diylbis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 68 | 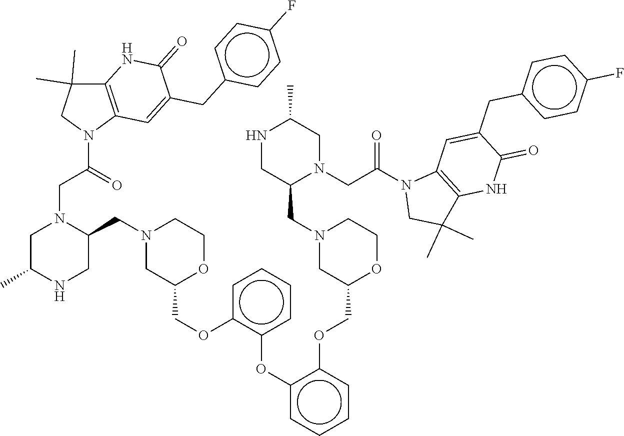 | 1,1′-(2,2′- ((3R,3′R,6R,6′R)- (((2S,2′S)- (((oxybis(2,1- phenylene))bis(oxy)) bis(methylene))bis (morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 69 | 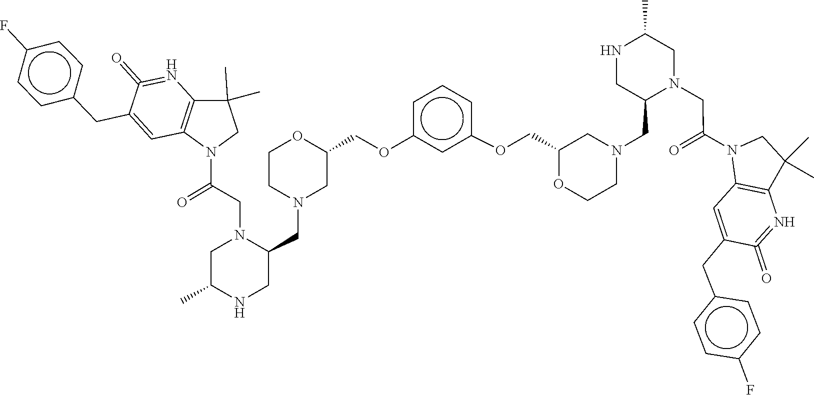 | 1,1′-(2,2′- ((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 70 | 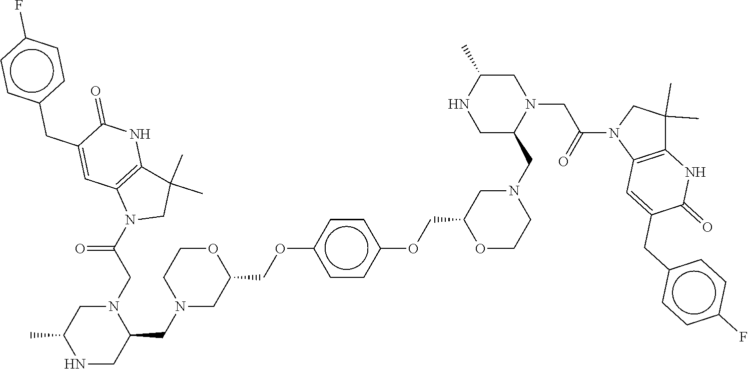 | 1,1′-(2,2′- ((3R,3′R,6R,6′R)- (((2S,2′S)-((1,4- phenylenebis(oxy))bis (methylene))bis (morpholine-2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 71 | 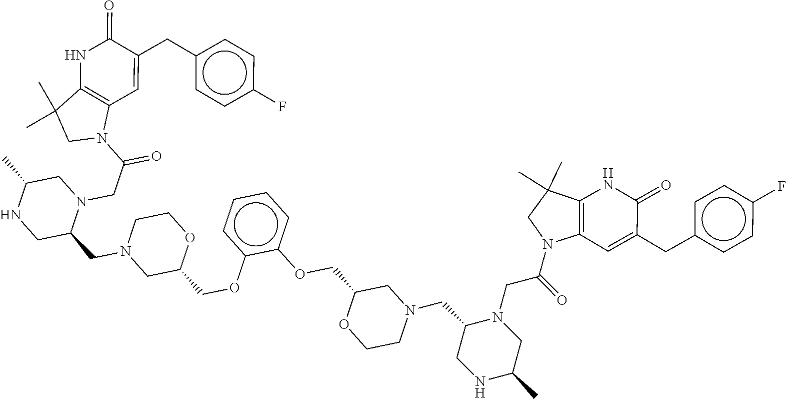 | 1,1′-(2,2′- ((3R,3′R,6R,6′R)- (((2S,2′S)-((1,2- phenylenebis(oxy))bis (methylene))bis (morpholine-2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 72 | 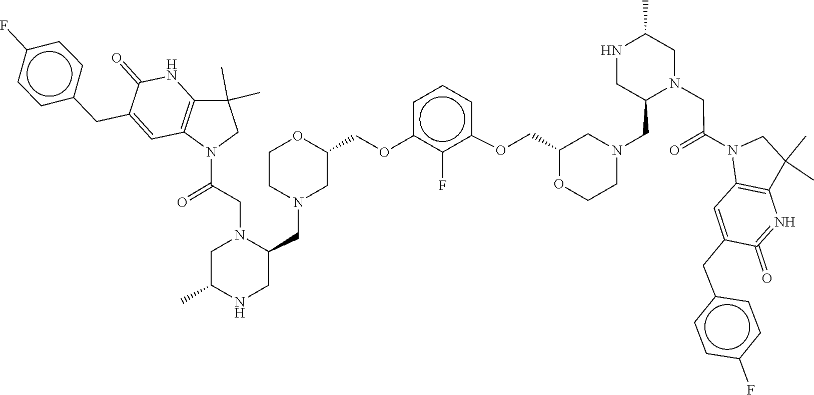 | 1,1′-(2,2′- ((3R,3′R,6R,6′R)- (((2S,2′S)-(((2-fluoro- 1,3- phenylene)bis(oxy))bis (methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 73 | 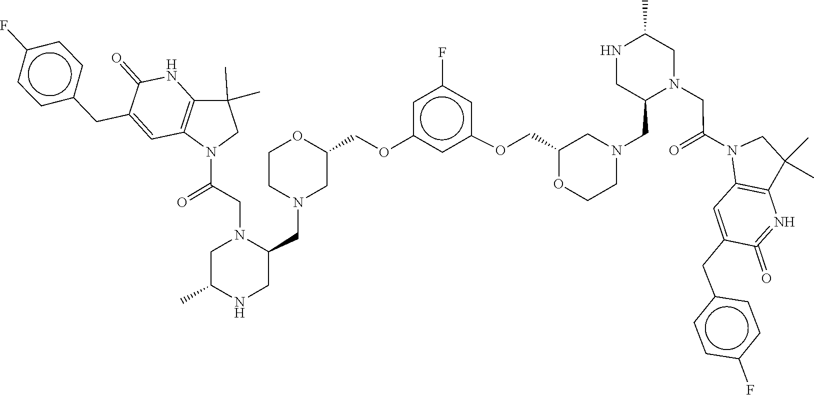 | 1,1′-(2,2′- ((3R,3′R,6R,6′R)- (((2S,2′S)-(((5-fluoro- 1,3- phenylene)bis(oxy))bis (methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 74 | 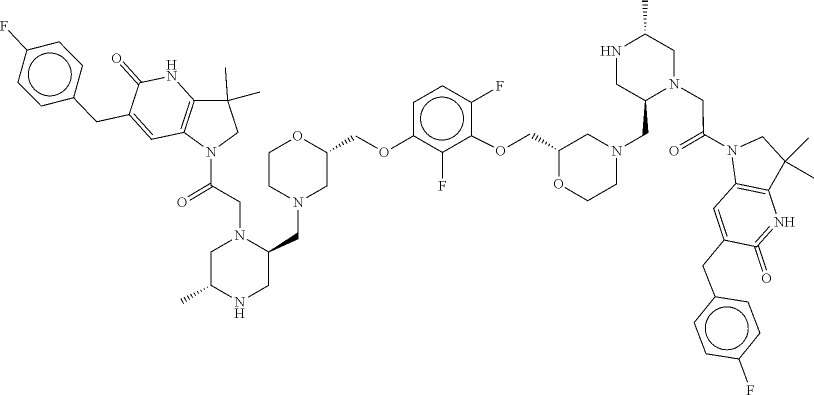 | 1,1′-(2,2′- ((3R,3′R,6R,6′R)- (((2S,2′S)-(((2,4- difluoro-1,3- phenylene)bis(oxy))bis (methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 75 | 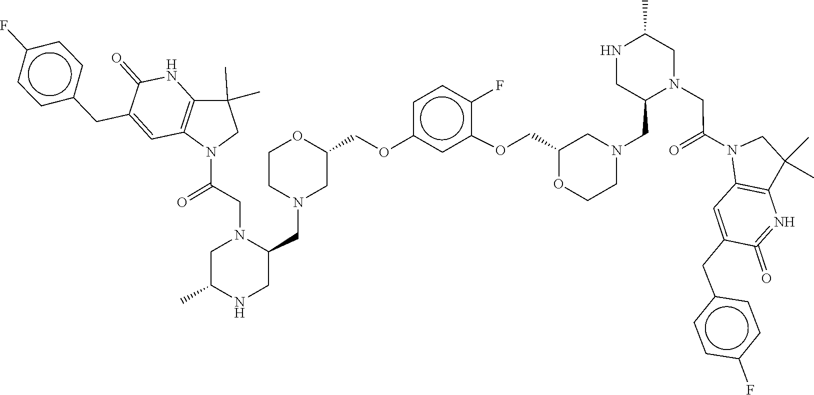 | 1,1′-(2,2′- ((3R,3′R,6R,6′R)- (((2S,2′S)-(((4-fluoro- 1,3- phenylene)bis(oxy))bis (methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 76 | 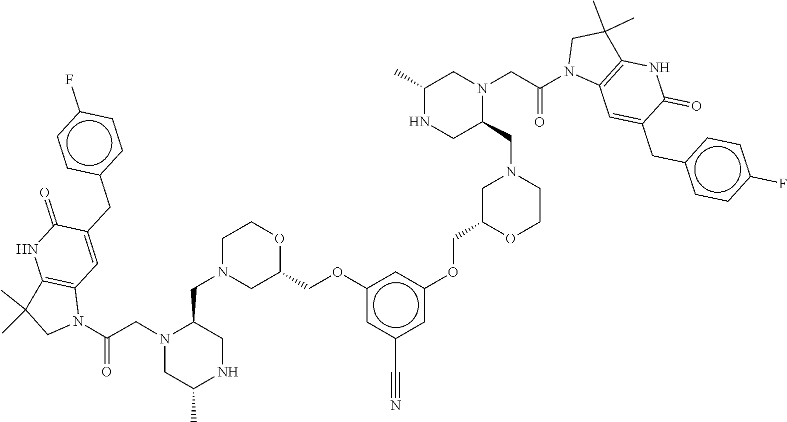 | 3,5-bis(((S)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholin-2- yl)methoxy)benzonitrile |
| 77 | 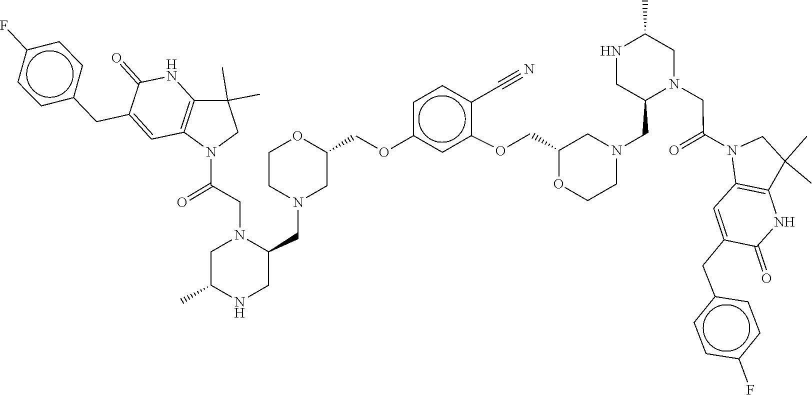 | 2,4-bis(((S)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholin-2- yl)methoxy)benzonitrile |
| 78 | 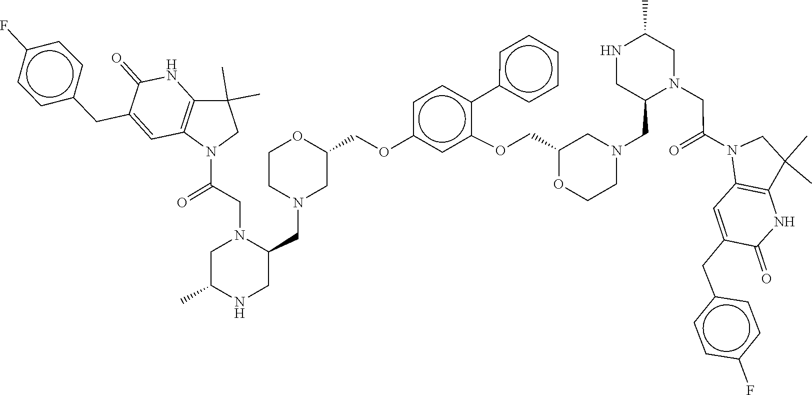 | 1,1′-(2,2′- ((3R,3′R,6R,6′R)- (((2S,2′S)-(([1,1′- biphenyl]-2,4- diylbis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 79 | 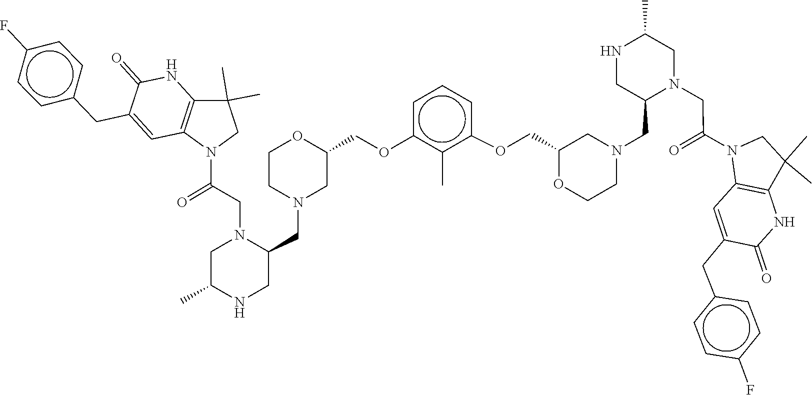 | 1,1′-(2,2′- ((3R,3′R,6R,6′R)- (((2S,2′S)-(((2-methyl- 1,3- phenylene)bis(oxy))bis (methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 80 | 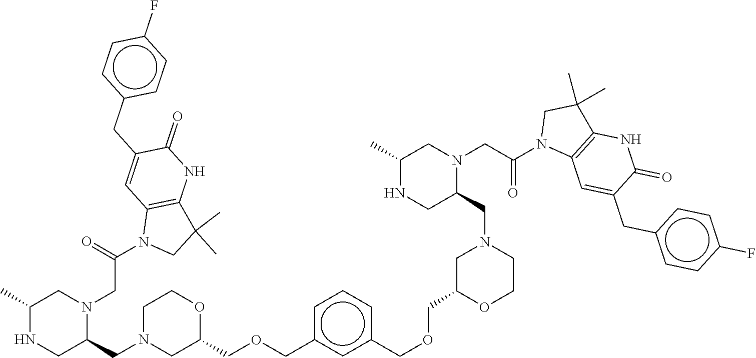 | 1,1′-(2,2′- ((3R,3′R,6R,6′R)- (((2S,2′S)-(((1,3- phenylenebis (methylene))bis (oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 81 | 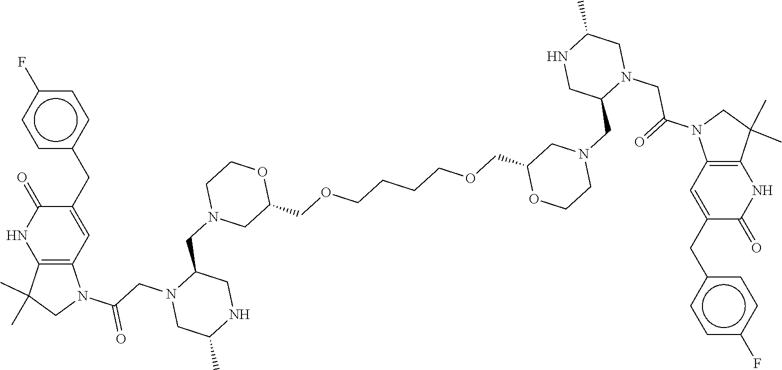 | 1,1′-(2,2′- ((3R,3′R,6R,6′R)- (((2S,2′S)-((butane-1,4- diylbis(oxy))bis(methylene)) bis(morpholine- 2,4. diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 82 | 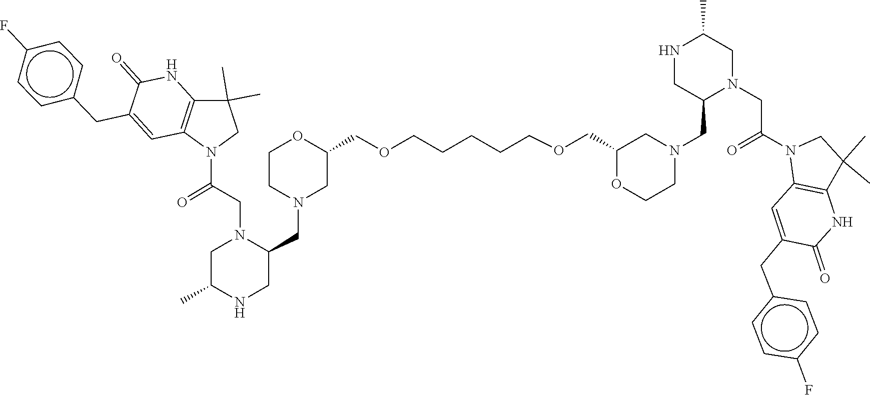 | 1,1′-(2,2′- ((3R,3′R,6R,6′R)- (((2S,2′S)-((pentane- 1,5- diylbis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 83 | 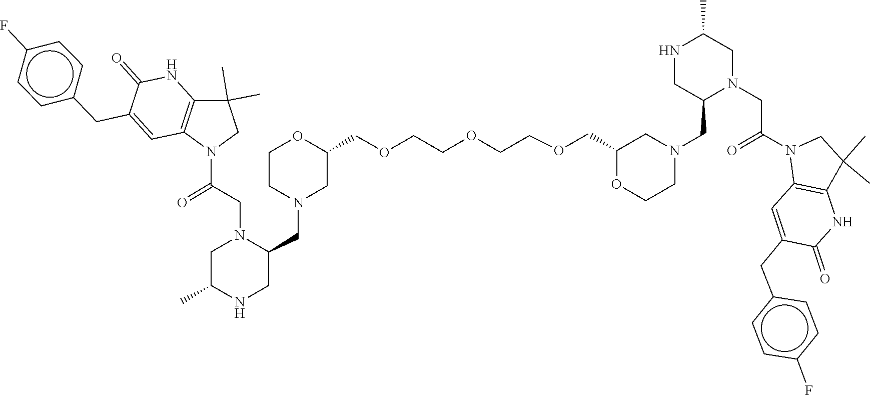 | 1,1′-(2,2′- ((3R,3′R,6R,6′R)- (((2S,2′S)- (((oxybis(ethane-2,1- diyl))bis(oxy))bis (methylene))bis (morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 84 | 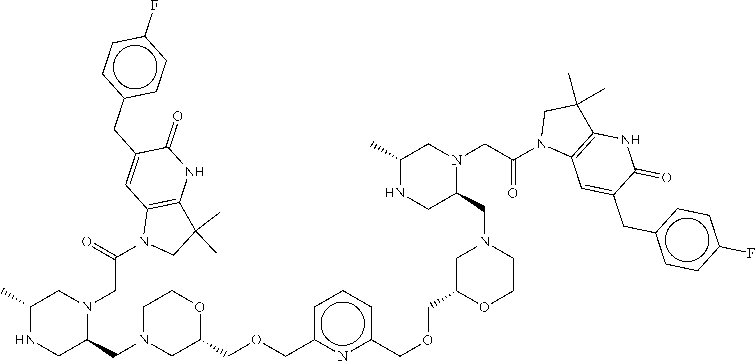 | 1,1′-(2,2′- ((3R,3′R,6R,6′R)- (((2S,2′S)-(((pyridine- 2,6- diylbis(methylene))bis (oxy))bis(methylene)) bis(morpholine-2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 85 | 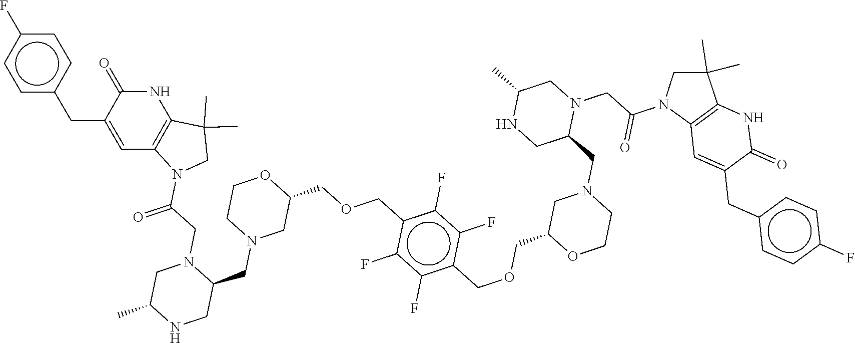 | 1,1′-(2,2′- ((3R,3′R,6R,6′R)- (((2S,2′S)-((((perfluoro- 1,4- phenylene)bis (methylene))bis(oxy)) bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 86 | 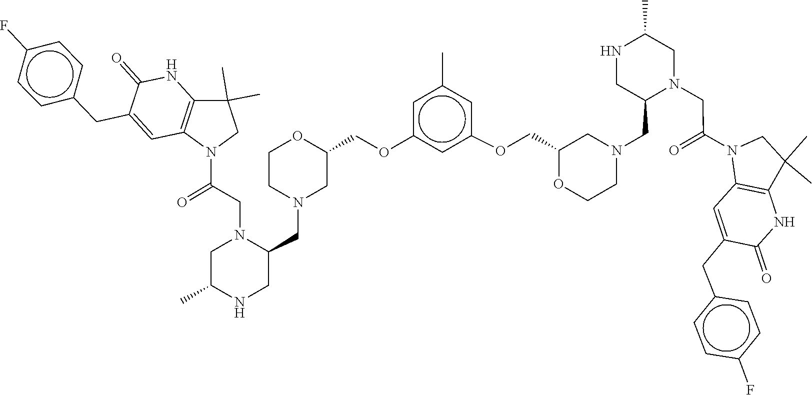 | 1,1′-(2,2′- ((3R,3′R,6R,6′R)- (((2S,2′S)-(((5-methyl- 1,3- phenylene)bis(oxy))bis (methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 87 | 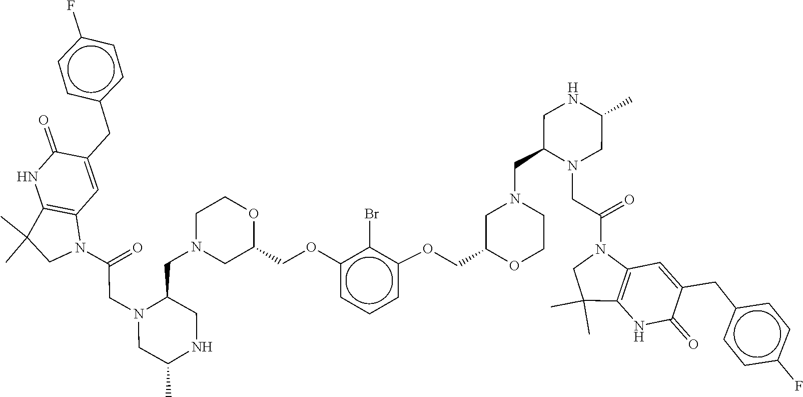 | 1,1′-(2,2′- ((3R,3′R,6R,6′R)- (((2S,2′S)-(((2-bromo- 1,3- phenylene)bis(oxy))bis (methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 88 | 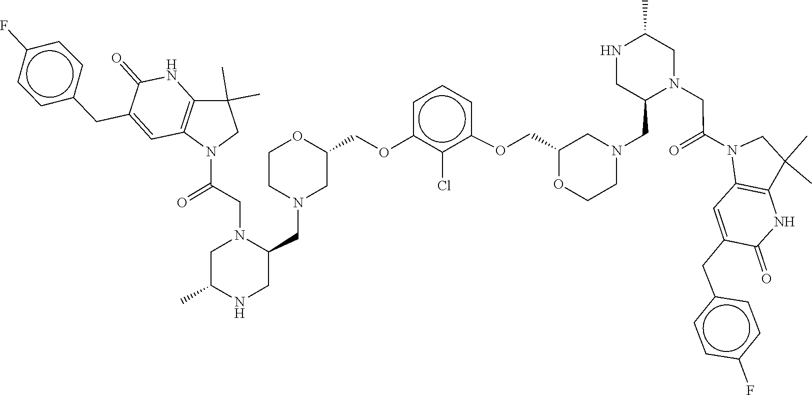 | 1,1′-(2,2′- ((3R,3′R,6R,6′R)- (((2S,2′S)-(((2-chloro- 1,3- phenylene)bis(oxy))bis (methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 89 | 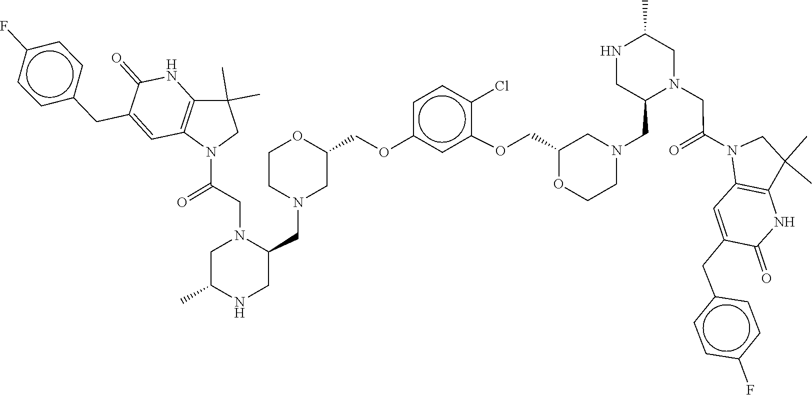 | 1,1′-(2,2′- ((3R,3′R,6R,6′R)- (((2S,2′S)-(((4-chloro- 1,3- phenylene)bis(oxy))bis (methylene))bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 90 | 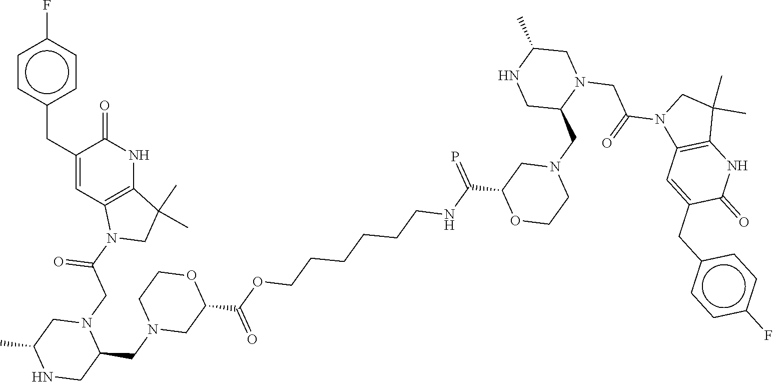 | (2S,2′S)-N,N-(hexane- 1,6-diyl)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 91 | 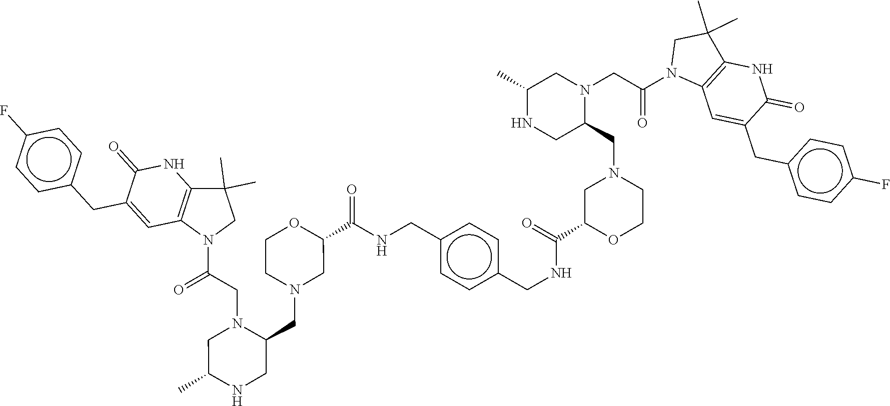 | (2S,2′S)-N,N-(1,4- phenylenebis(methylene)) bis(4-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 92 | 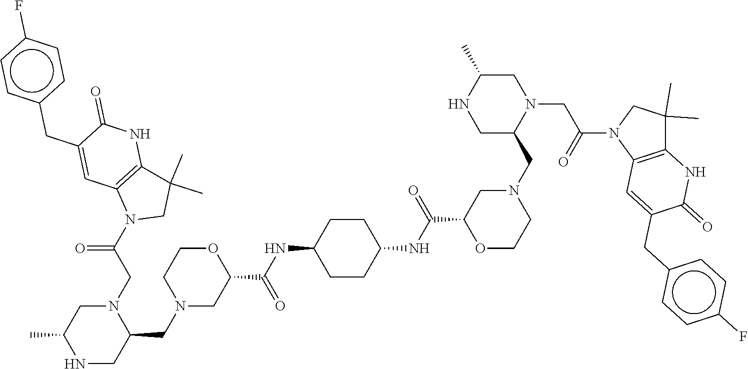 | (2S,2′S)-N,N-((1S,4S)- cyclohexane-1,4- diyl)bis(4-(((2R,5R)-1- (2-(6-(4-fluorobenzyl)- 3,3-dimethyl-5-oxo- 2,3,4,5-tetrahydro-1/- pyrrolo[3,2-b]pyridin- 1-yl)-2-oxoethyl)-5- methylpiperazin-2- yl)methyl)morpholine- 2-carboxamide) |
| 93 | 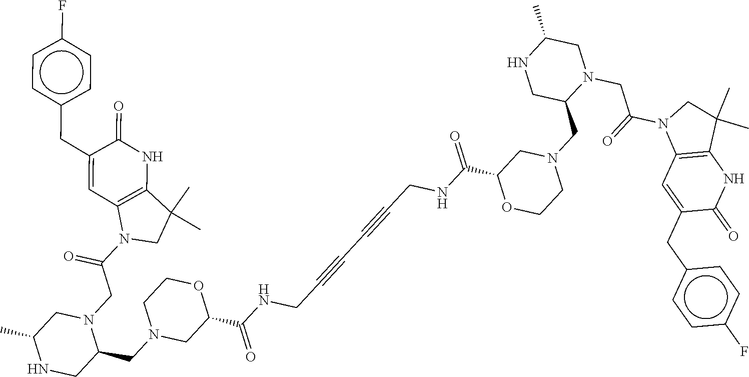 | (2S,2′S)-N,N-(hexa- 2,4-diyne-1,6- diyl)bis(4-(((2R,5R)-1- (2-(6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 94 | 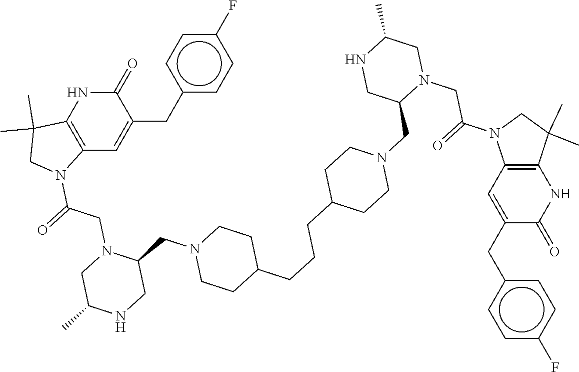 | 1,1′-(2,2′- ((3R,3′R,6R,6′R)- ((propane-1,3- diylbis(piperidine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 95 | 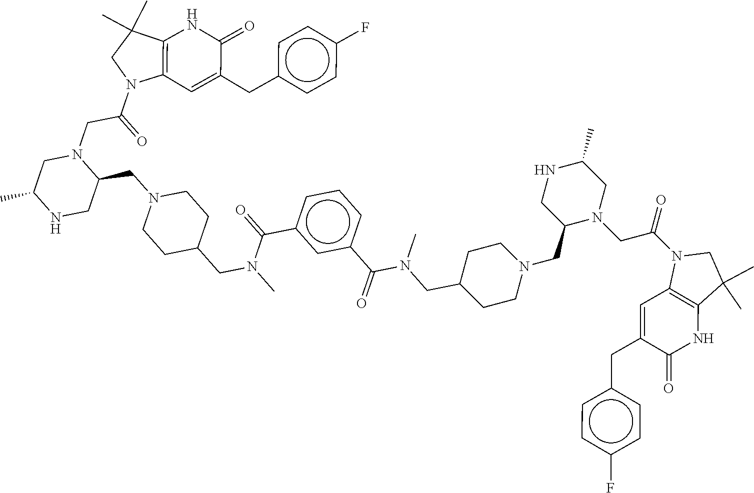 | N1,N3-bis((1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)methyl)-N1,N3- dimethylisophthalamide |
| 96 | 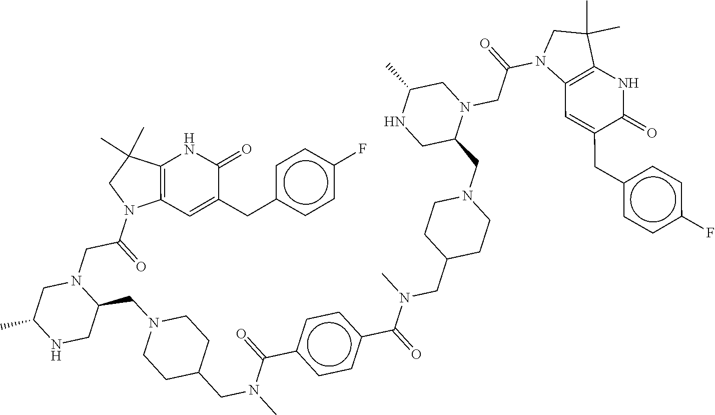 | N1,N4-bis((1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)methyl)-N1,N4- dimethylterephthalamide |
| 97 | 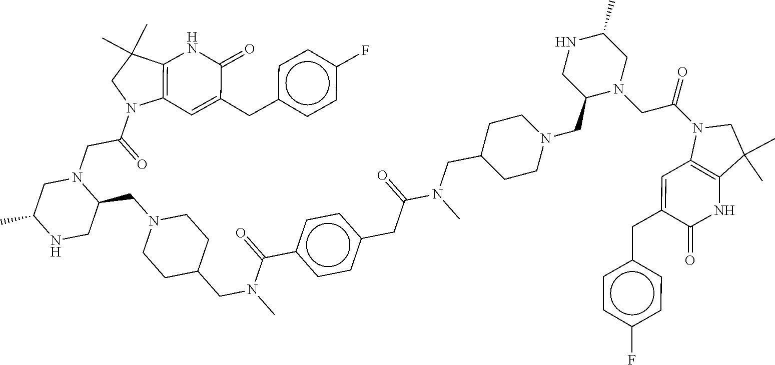 | N-((1-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)methyl)-4-(2-(((1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)methyl)(methyl)amino)- 2-oxoethyl)-N- methylbenzamide |
| 98 | 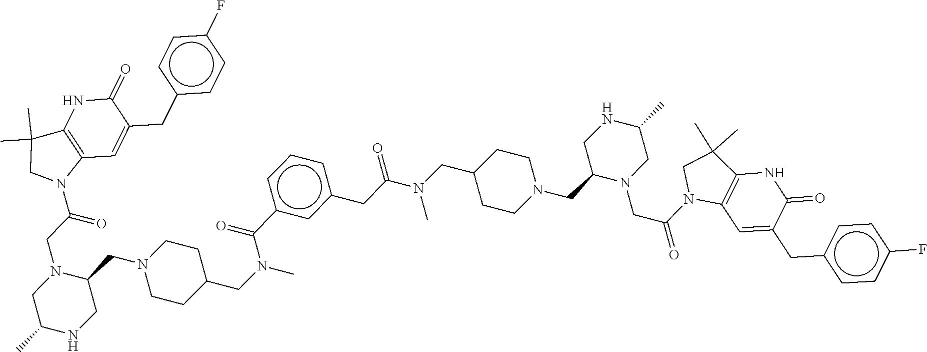 | N-((1-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)methyl)-3-(2-(((1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)methyl)(methyl) amino)-2-oxoethyl)-N- methylbenzamide |
| 99 | 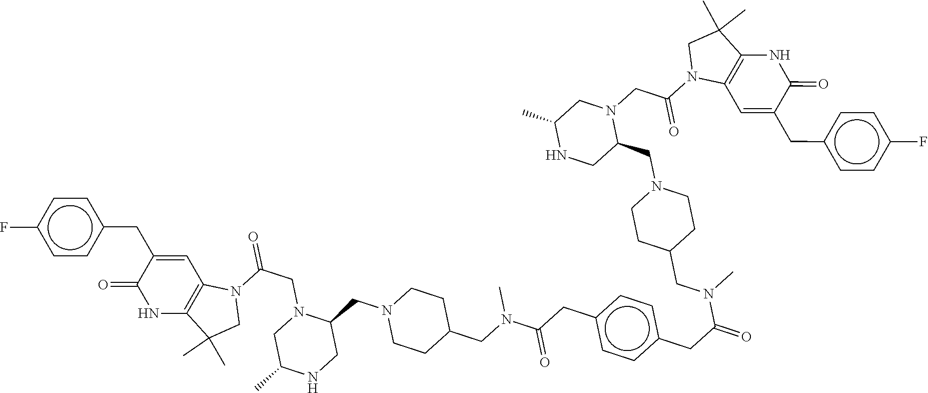 | 2,2′-(1,4- phenylene)bis(N-((1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)methyl)-N- methylacetamide) |
| 100 | 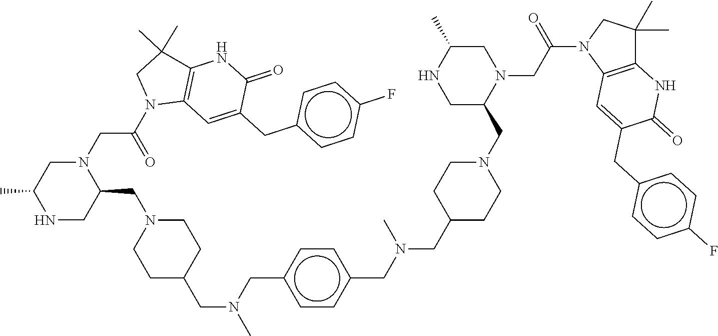 | 1,1′-(2,2′- ((3R,3′R,6R,6′R)- (((((1,4- phenylenebis(methylen e))bis(methylazanediyl)) bis(methylene))bis (piperidine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 101 | 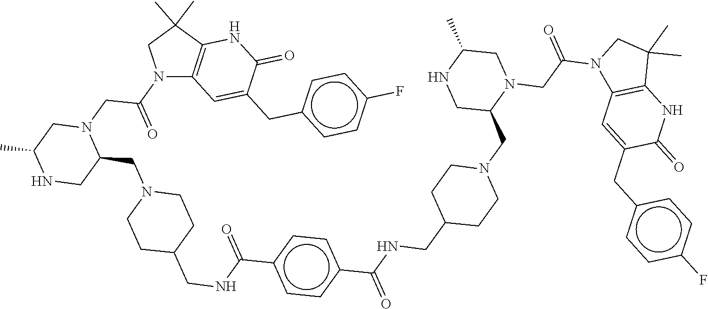 | N1,N4-bis((1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)methyl) terephthalamide |
| 102 | 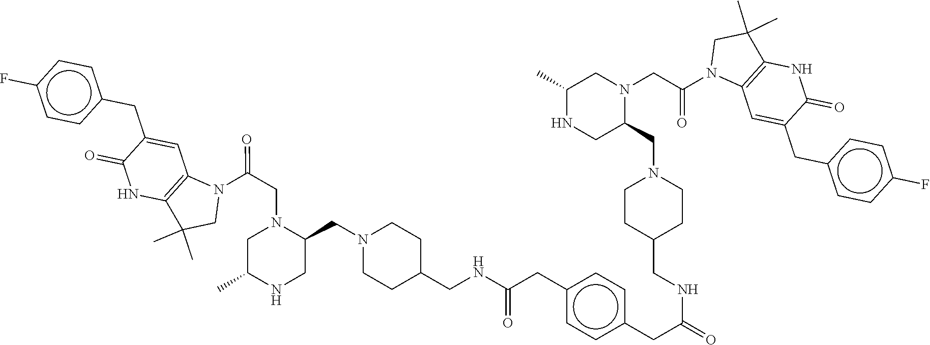 | 2,2′-(1,4- phenylene)bis(N-((1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)methyl)acetamide) |
| 103 | 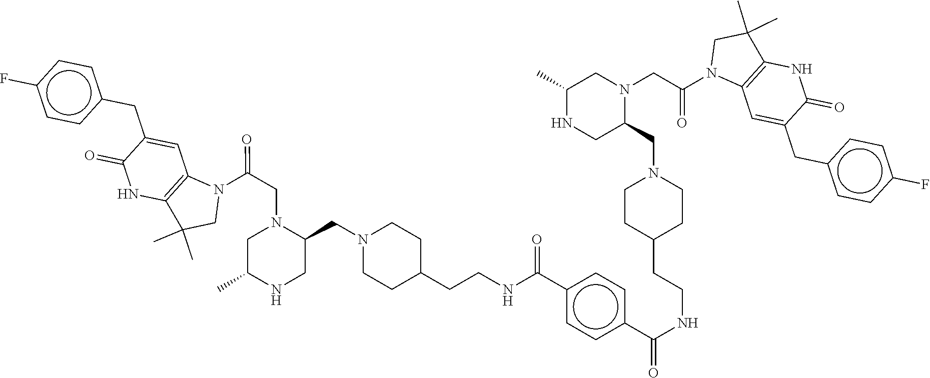 | N1,N4-bis(2-(1- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)ethyl)terephthalamide |
| 104 | 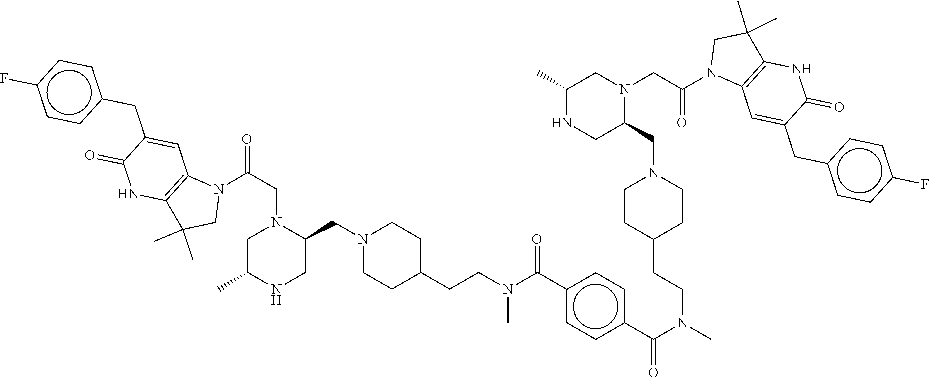 | N1,N4-bis(2-(1- (((2R,5R)-1-(2-(6-(4- fluorobenzyl)-3,3- dimethyl-5-oxo- 2,3,4,5-tetrahydro-1//- pyrrolo[3,2-b]pyridin- 1-yl)-2-oxoethyl)-5- methylpiperazin-2- yl)methyl)piperidin-4- yl)ethyl)-N1,N4- dimethylterephthalamide |
| 105 | 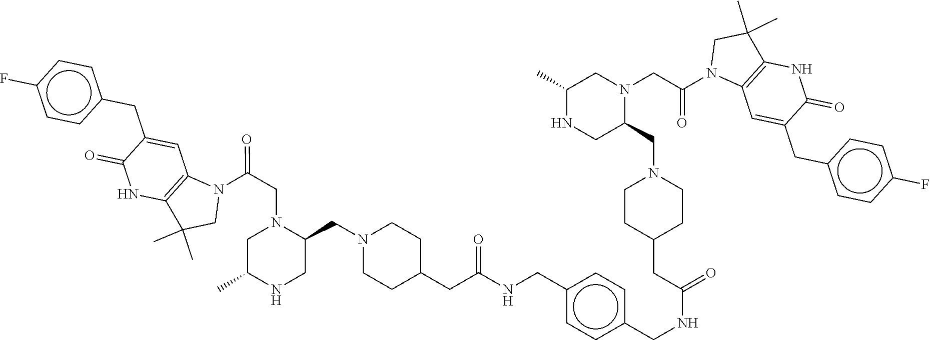 | N,N-(1,4- phenylenebis(methylene)) bis(2-(1-(((2R,5R)-1- (2-(6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)acetamide) |
| 106 | 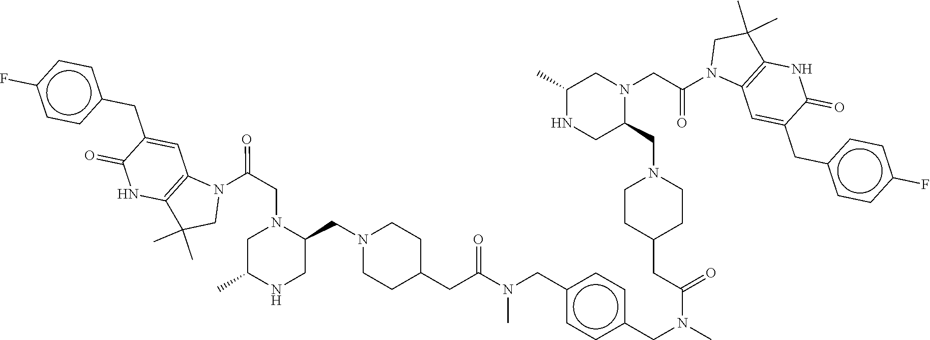 | N,N-(1,4- phenylenebis(methylene)) bis(2-(1-(((2R,5R)-1- (2-(6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)piperidin-4- yl)-N- methylacetamide) |
| 107 | 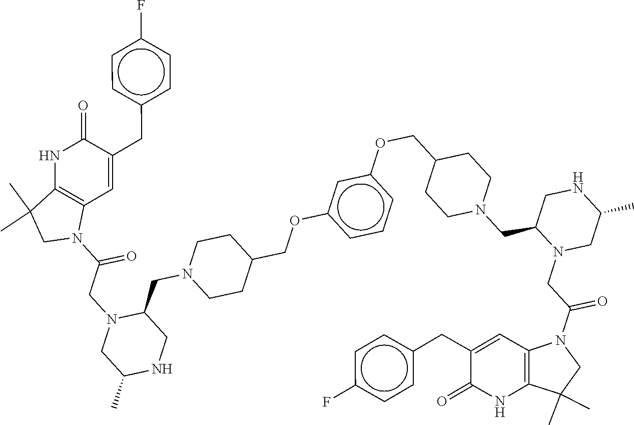 | 1,1′-(2,2′- ((3R,3′R,6R,6′R)- ((((1,3- phenylenebis(oxy))bis (methylene))bis (piperidine-4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 108 | 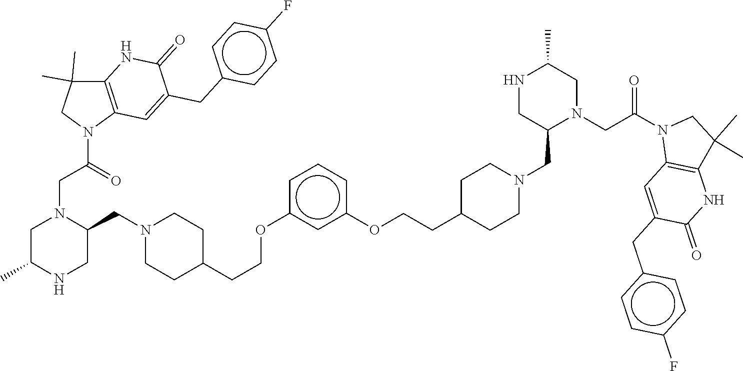 | 1,1′-(2,2′- ((3R,3′R,6R,6′R)- ((((1,3- phenylenebis(oxy))bis (ethane-2,1- diyl))bis(piperidine- 4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 109 | 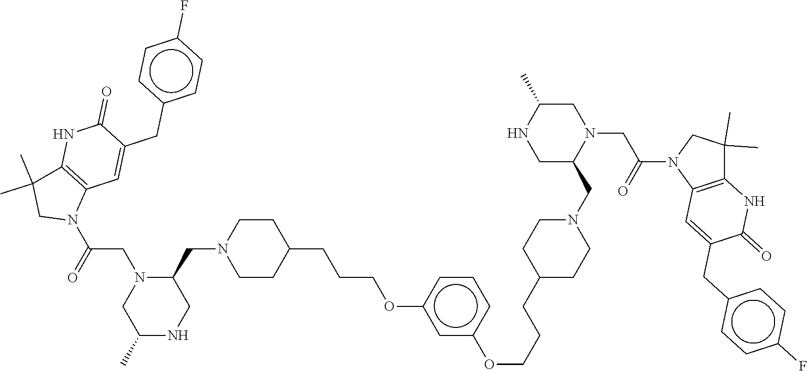 | 1,1′-(2,2′- ((3R,3′R,6R,6′R)- ((((1,3- phenylenebis(oxy))bis (propane-3,1- diyl))bis(piperidine- 4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 110 | 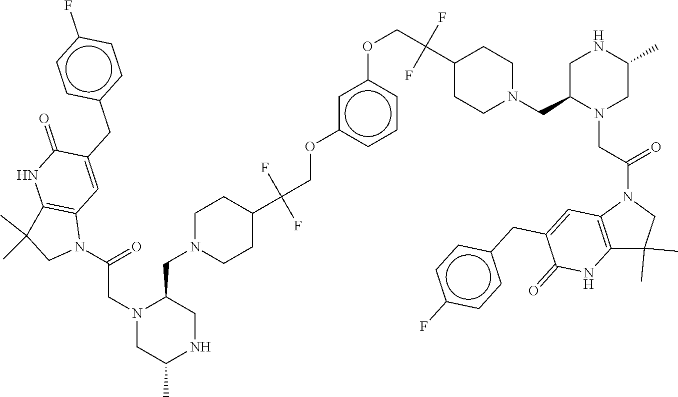 | 1,1′-(2,2′- ((3R,3′R,6R,6′R)- ((((1,3- phenylenebis(oxy))bis (1,1-difluoroethane-2,1- diyl))bis(piperidine- 4,1- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 111 | 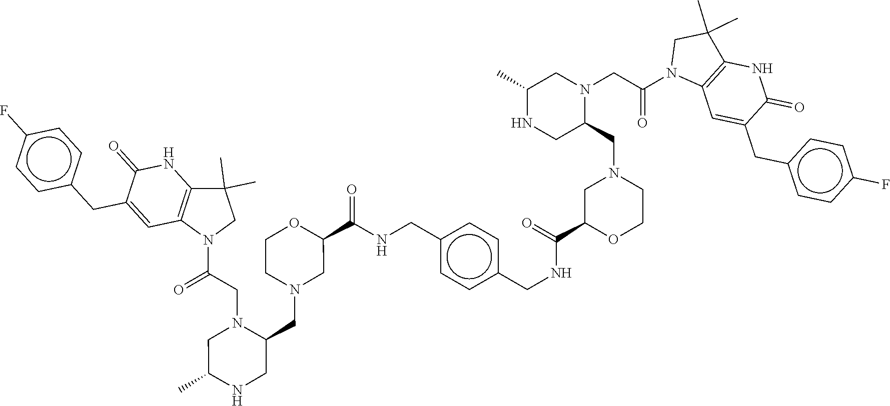 | (2R,2′R)-N,N′-(1,4- phenylenebis(methylen e))bis(4-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 112 | 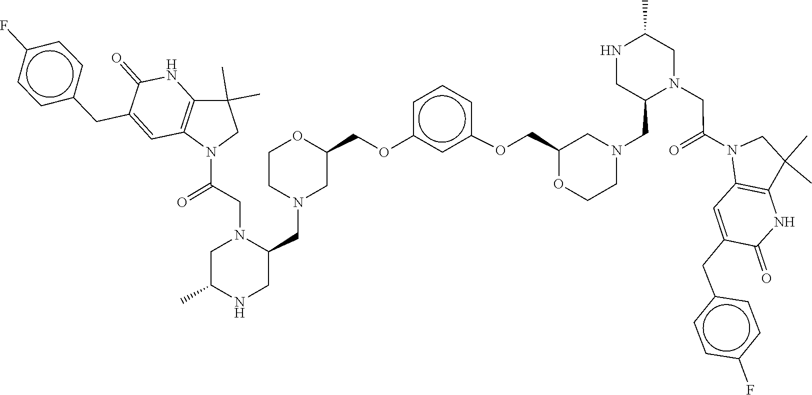 | 1,1′-(2,2′- ((3R,3′R,6R,6′R)- (((2R,2′R)-((1,3- phenylenebis(oxy))bis (methylene))bis(morpho line-2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 113 | 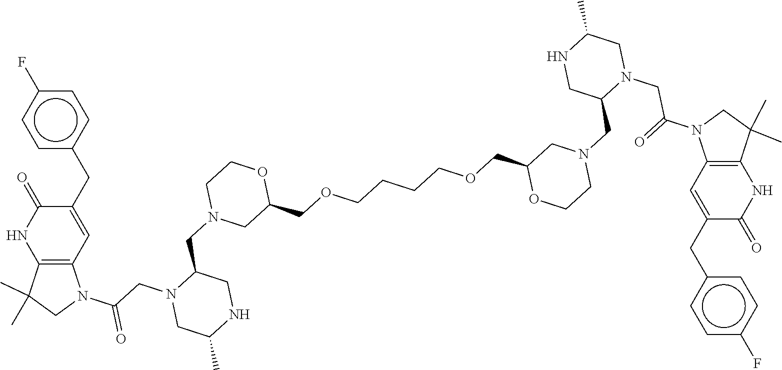 | 1,1′-(2,2′- ((3R,3′R,6R,6′R)- (((2R,2′R)-((butane-1,4- diylbis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 114 | 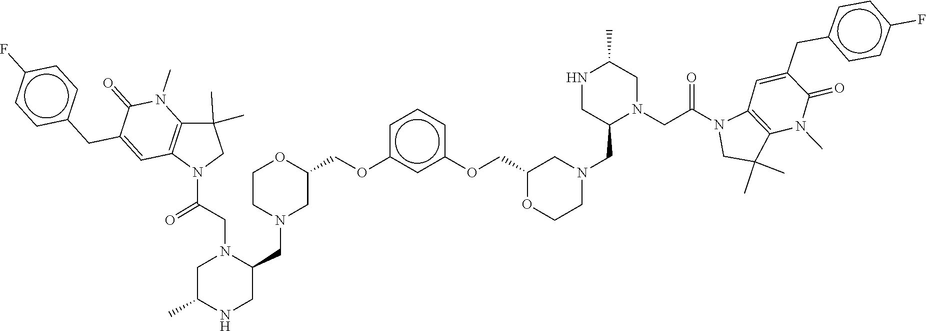 | 1,1′-(2,2′- ((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1- diyl))bis(acetyl))bis(6- (4-fluorobenzyl)-3,3,4- trimethyl-1,2,3,4- tetrahydro-5H- pyrrolo[3,2-b]pyridin- 5-one) |
| 115 | 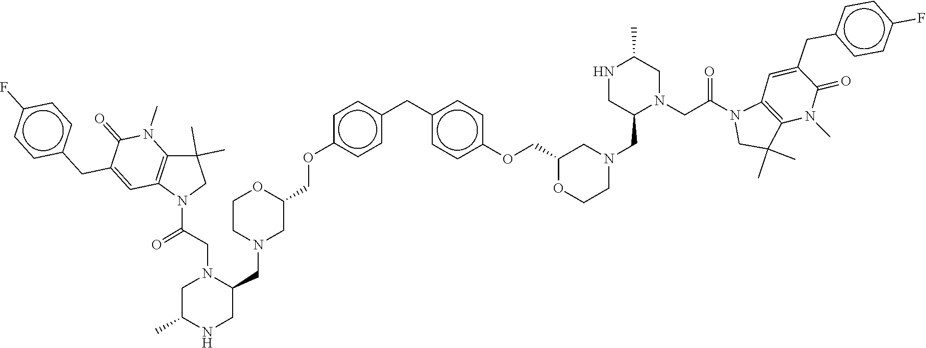 | 1,1′-(2,2′- ((3R,3′R,6R,6′R)- (((2S,2′S)- (((methylenebis(4,1- phenylene))bis(oxy)) bis(methylene))bis (morpholine- 2,4- diyl))bis(methylene)) bis(3-methylpiperazine- 6,1- diyl))bis(acetyl))bis(6- (4-fluorobenzyl)-3,3,4- trimethyl-1,2,3,4- tetrahydro-5H- pyrrolo[3,2-b]pyridin- 5-one) |
| 116 | 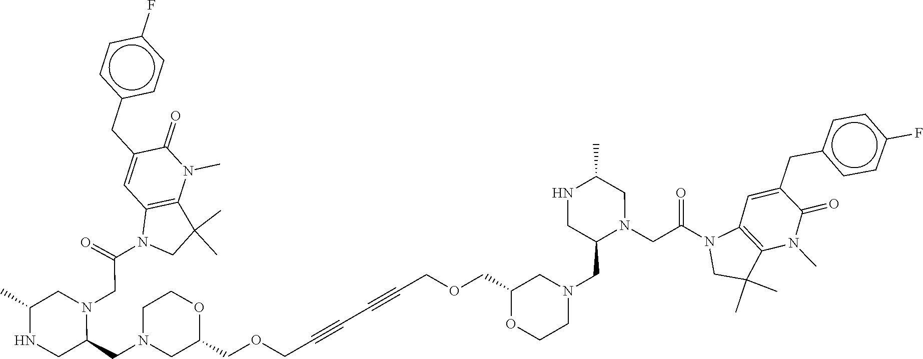 | 1,1′-(2,2′- ((3R,3′R,6R,6′R)- (((2S,2′S)-((hexa-2,4- diyne-1,6- diylbis(oxy))bis(methylene)) bis(morpholine- 2,4- diyl))bis(methylene)) bis(3-methylpiperazine- 6,1- diyl))bis(acetyl))bis(6- (4-fluorobenzyl)-3,3,4- trimethyl-1,2,3,4- tetrahydro-5H- pyrrolo[3,2-b]pyridin- 5-one) |
| 117 | 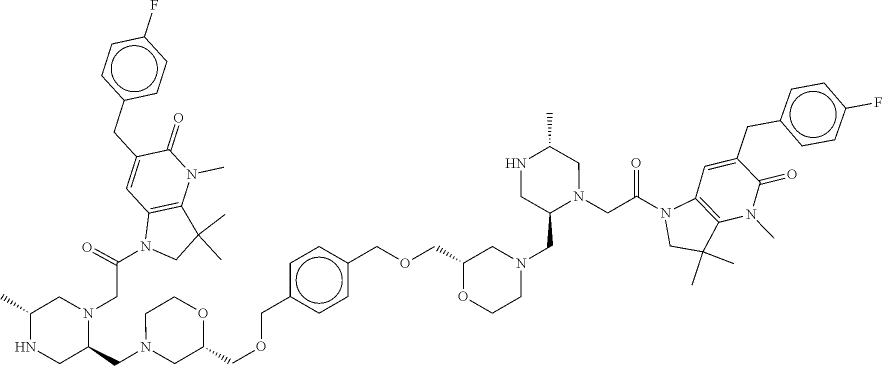 | 1,1′-(2,2′- ((3R,3′R,6R,6′R)- (((2S,2′S)-(((1,4- phenylenebis (methylene))bis (oxy))bis (methylene))bis (morpholine- 2,4- diyl))bis(methylene)) bis(3-methylpiperazine- 6,1- diyl))bis(acetyl))bis(6- (4-fluorobenzyl)-3,3,4- trimethyl-1,2,3,4- tetrahydro-5H- pyrrolo[3,2-b]pyridin- 5-one) |
| 118 | 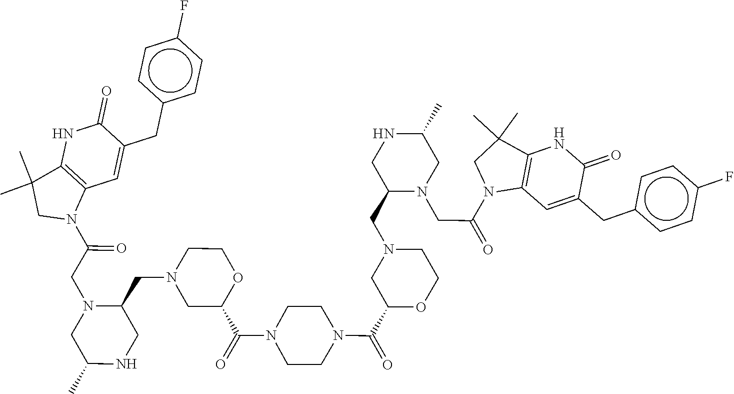 | 1,1′-(2,2′- ((3R,3′R,6R,6′R)- (((2S,2′S)-(piperazine- 1,4- dicarbonyl)bis (morpholine-2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 119 | 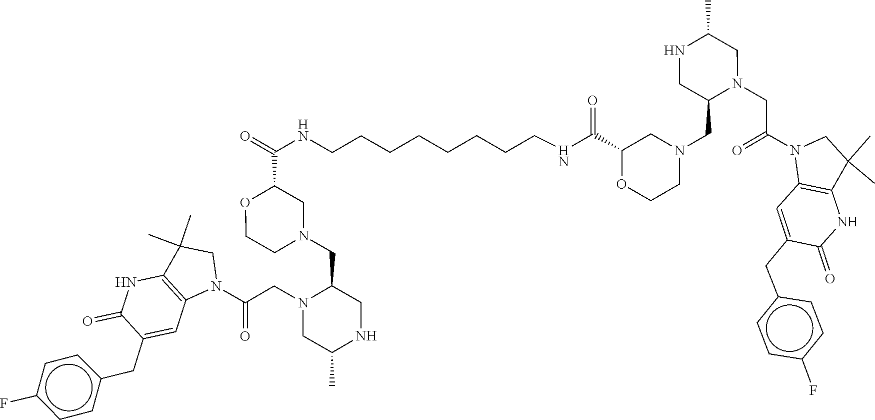 | (2S,2′S)-N,N-(octane- 1,8-diyl)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 120 | 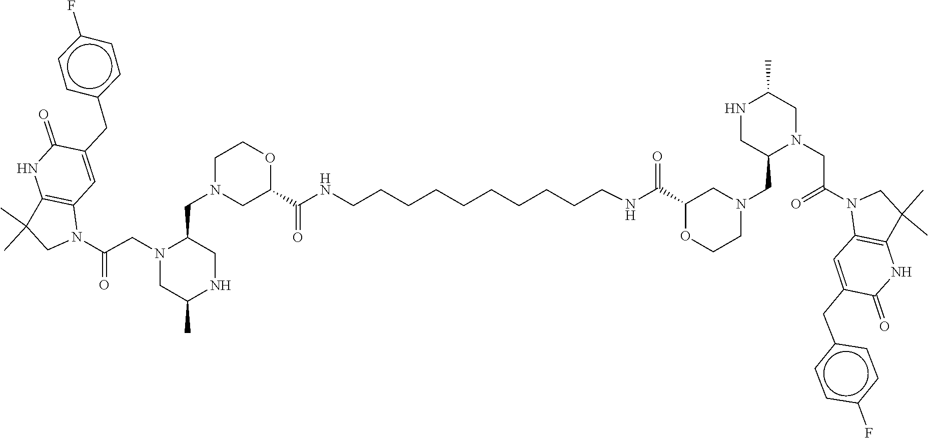 | (2S,2′S)-N,N-(decane- 1,10-diyl)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 121 | 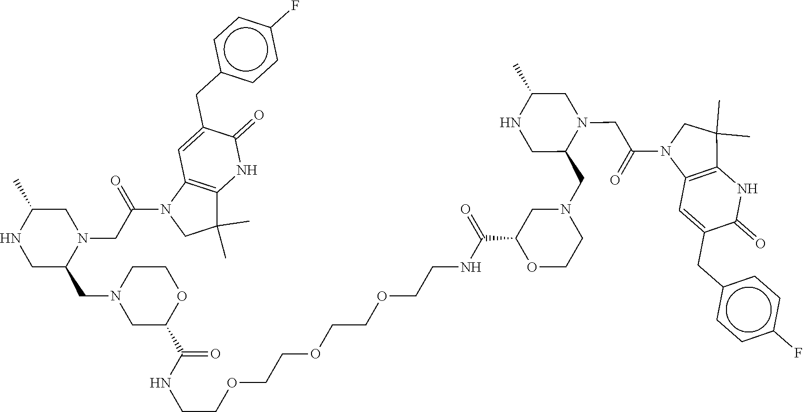 | (2S,2′S)-N,N′- (((oxybis(ethane-2,1- diyl))bis(oxy))bis(ethane- 2,1-diyl))bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 122 | 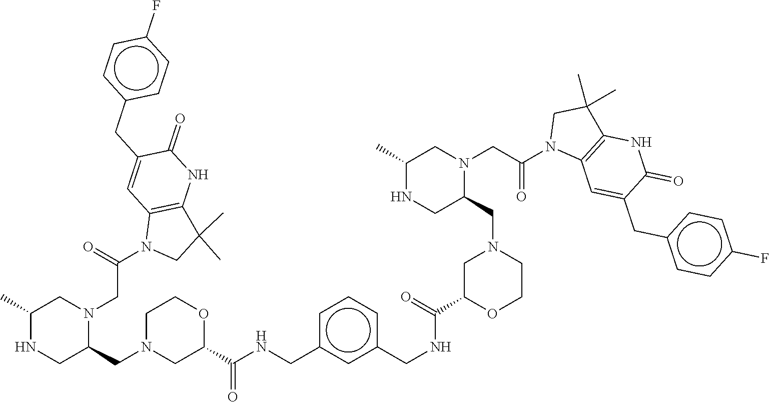 | (2S,2′S)-N,N-(1,3- phenylenebis(methylen e))bis(4-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 123 | 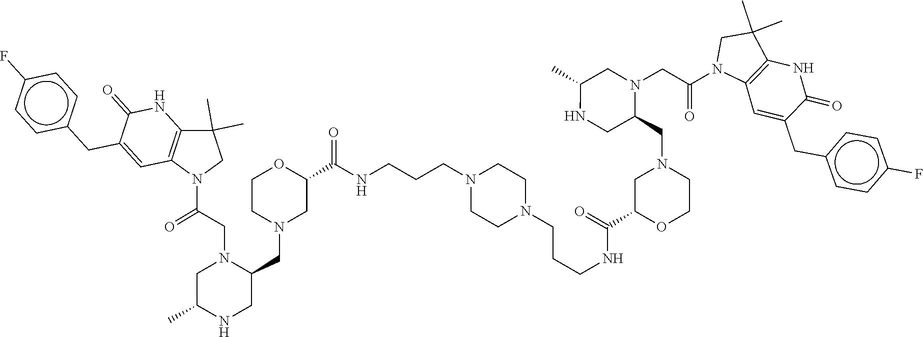 | (2S,2′S)-N,N′- (piperazine-1,4- diylbis(propane-3,1- diyl))bis(4-(((2R,5R)-1- (2-(6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 124 | 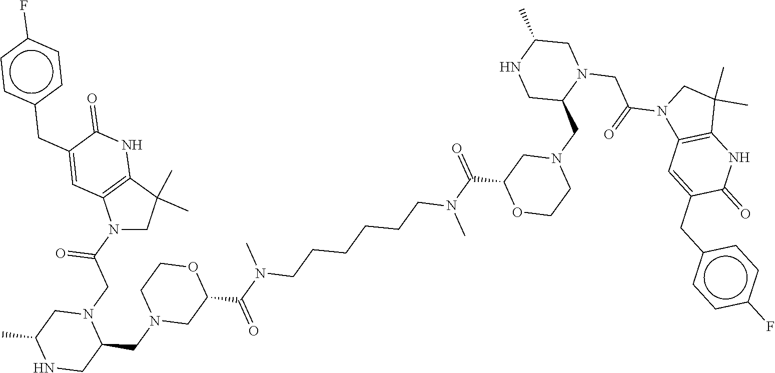 | (2S,2′S)-N,N′-(hexane- 1,6-diyl)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-N- methylmorpholine-2- carboxamide) |
| 125 | 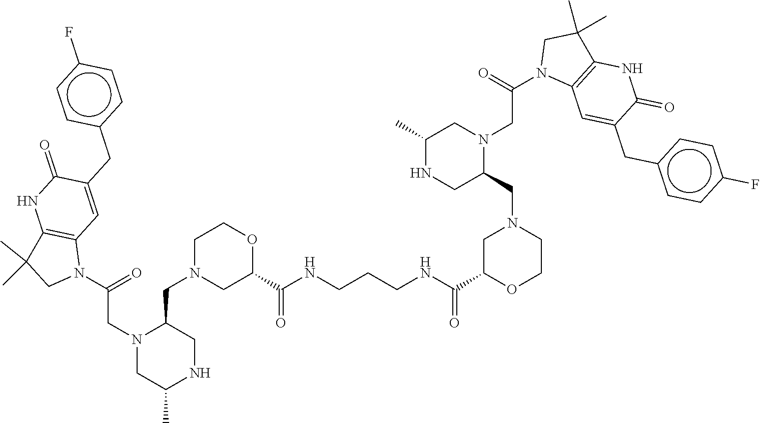 | (2S,2′S)-N,N′-(propane- 1,3-diyl)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 126 | 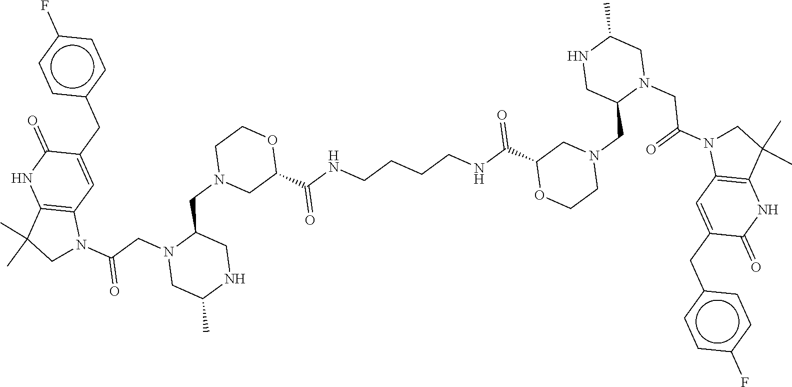 | (2S,2′S)-N,N-(butane- 1,4-diyl)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 127 | 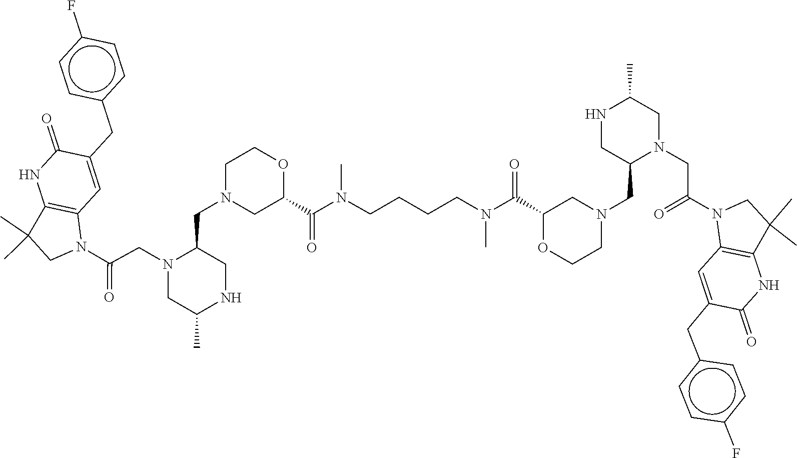 | (2S,2′S)-N,N′-(butane- 1,4-diyl)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-N- methylmorpholine-2- carboxamide) |
| 128 | 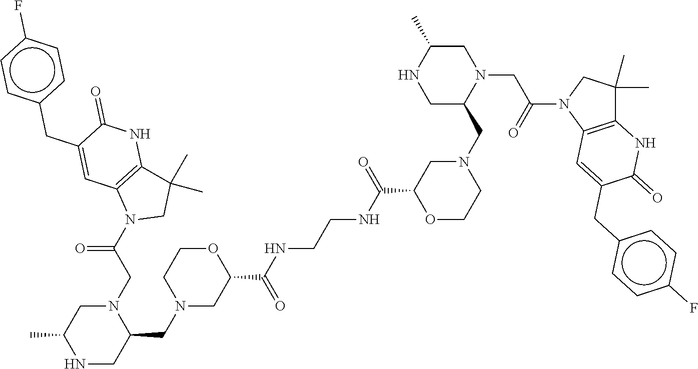 | (2S,2′S)-N,N′-(ethane- 1,2-diyl)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 129 | 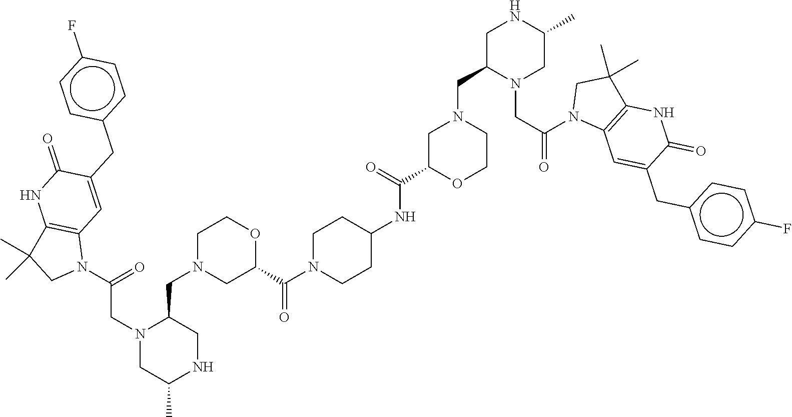 | (S)-4-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)-N-(1-((S)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carbonyl)piperidin-4- yl)morpholine-2- carboxamide |
| 130 | 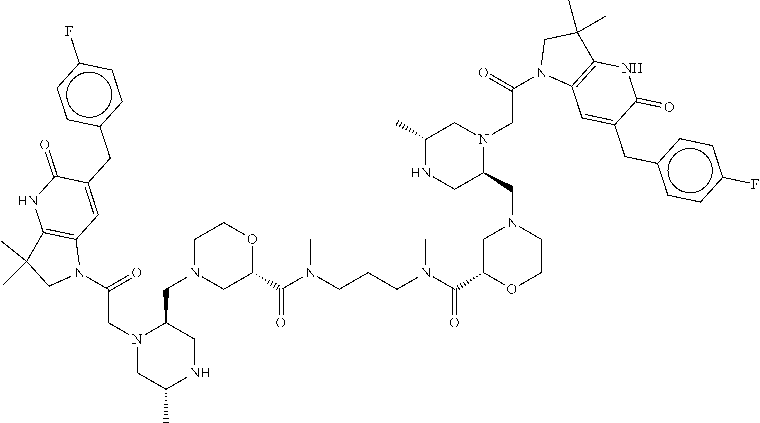 | (2S,2′S)-N,N-(propane- 1,3-diyl)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-N- methylmorpholine-2- carboxamide) |
| 131 | 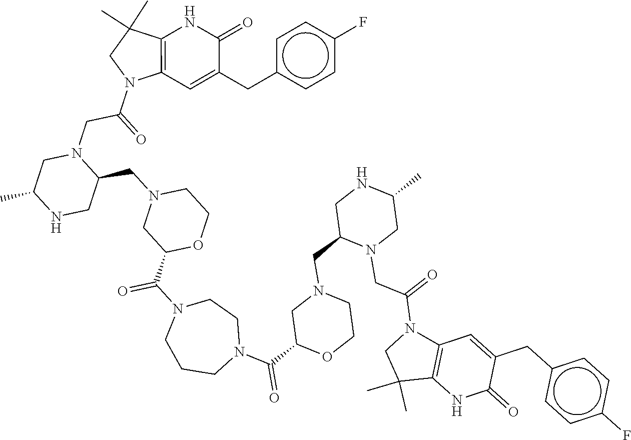 | 1,1′-(2,2′- ((3R,3′R,6R,6′R)- (((2S,2′S)-(1,4- diazepane-1,4- dicarbonyl)bis (morpholine-2,4- diyl))bis(methylene)) bis(3-methylpiperazine- 6,1- diyl))bis(acetyl))bis(6- (4-fluorobenzyl)-3,3- dimethyl-1,2,3,4- tetrahydro-5H- pyrrolo[3,2-b]pyridin- 5-one) |
| 132 | 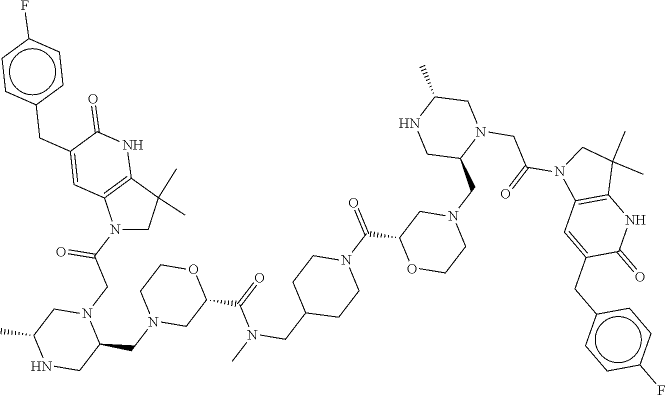 | (S)-4-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)-N-((1-((S)- 4-(((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carbonyl)piperidin-4- yl)methyl)-N- methylmorpholine-2- carboxamide |

| | | |
|---|---|---|
| 133 | 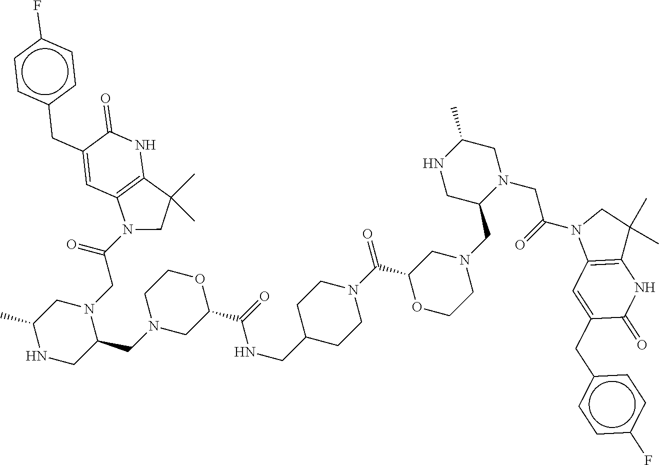 | (S)-4-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)-N-((1-((S)- 4-(((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carbonyl)piperidin-4- yl)methyl)morpholine- 2-carboxamide |
| 134 | 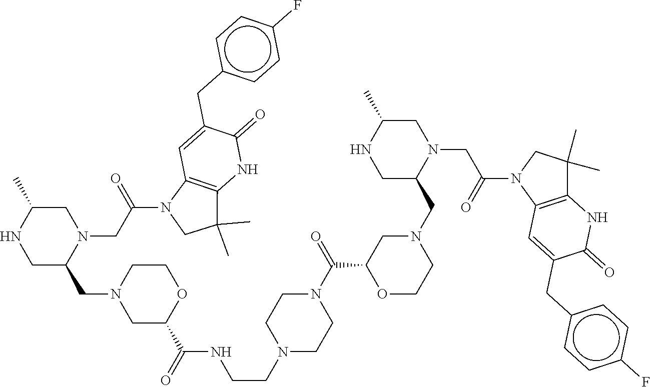 | (S)-4-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)-N-(2-(4- ((S)-4-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carbonyl)piperazin-1- yl)ethyl)morpholine-2- carboxamide |
| 135 | 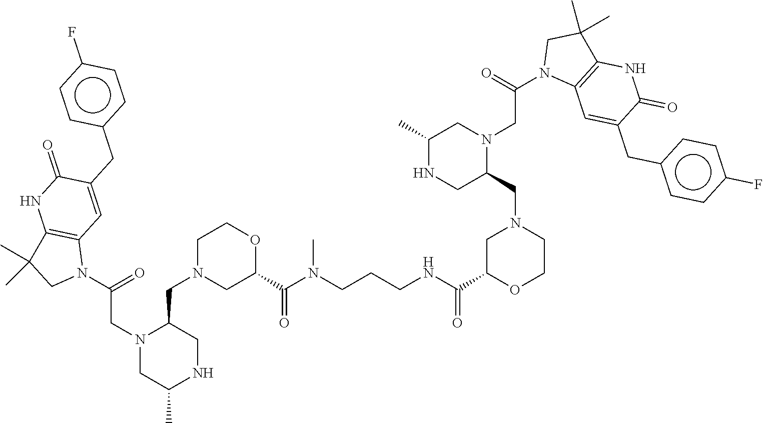 | (S)-4-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)-N-(3-((S)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2- carboxamido)propyl)- N-methylmorpholine-2- carboxamide |
| 136 | 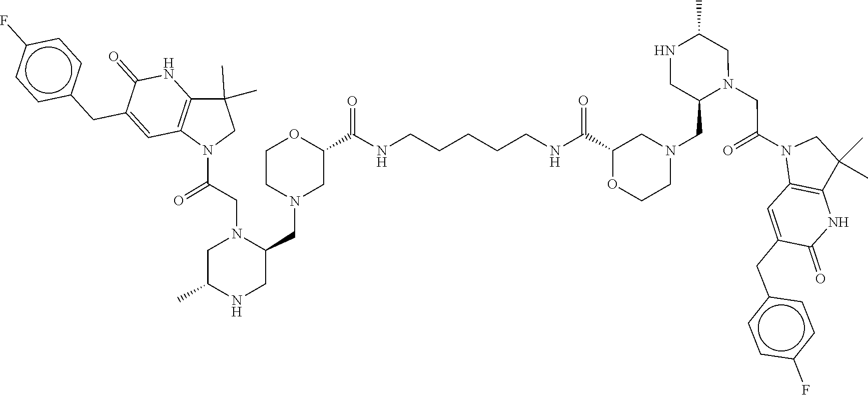 | (2S,2′S)-N,N′-(pentane- 1,5-diyl)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 137 | 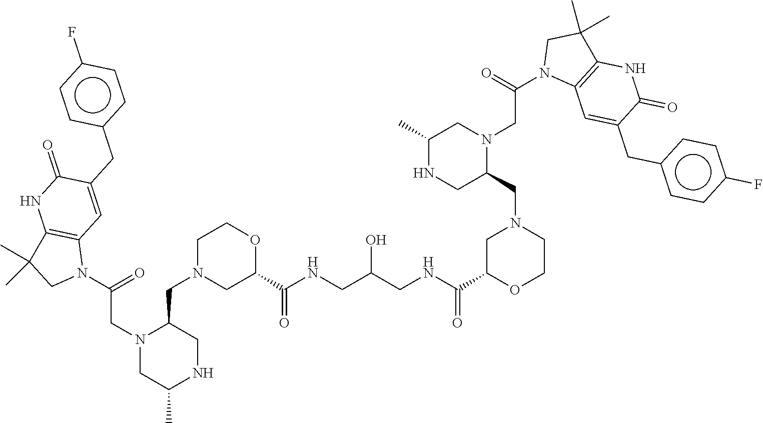 | (2S,2′S)-N,N′-(2- hydroxypropane-1,3- diyl)bis(4-(((2R,5R)-1- (2-(6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 138 | 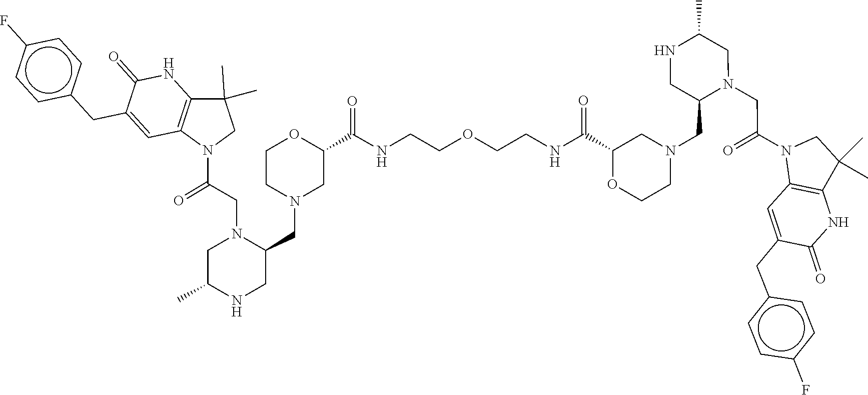 | (2S,2′S)-N,N′- (oxybis(ethane-2,1- diyl))bis(4-(((2R,5R)-1- (2-(6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 139 | 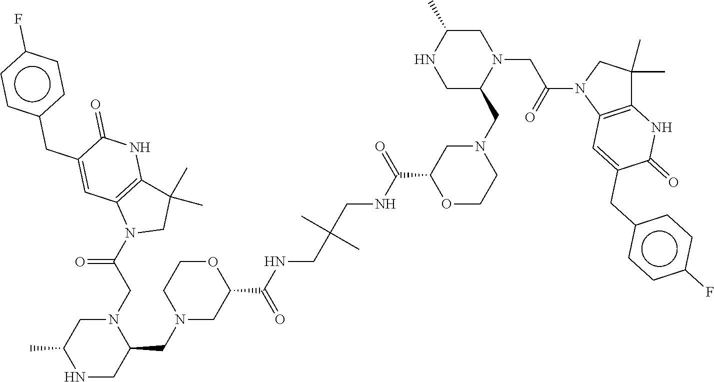 | (2S,2′S)-N,N′-(2,2- dimethylpropane-1,3- diyl)bis(4-(((2R,5R)-1- (2-(6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 140 | 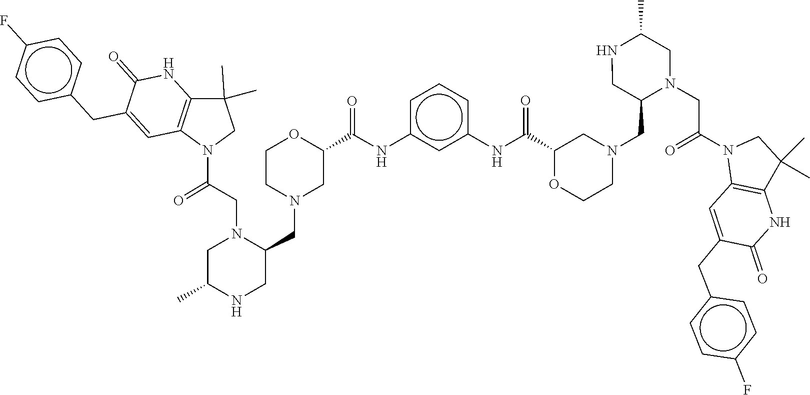 | (2S,2′S)-N,N′-(1,3- phenylene)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 141 | 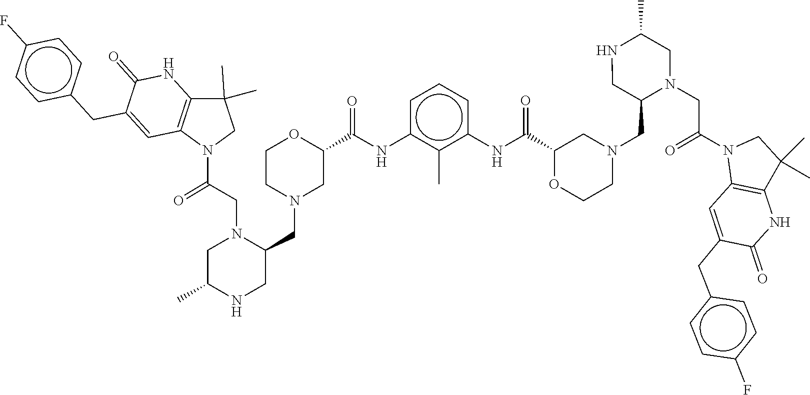 | (2S,2′S)-N,N′-(2- methyl-1,3- phenylene)bis(4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 142 | 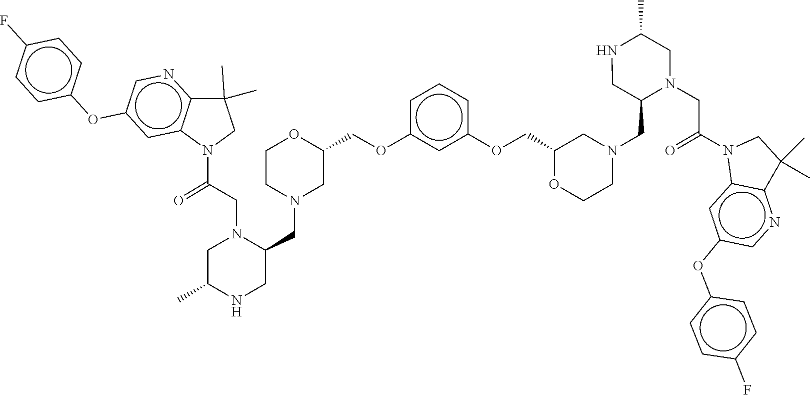 | 1-[6-(4- fluorophenoxy)-3,3- dimethyl-2H- pyrrolo[3,2-b]pyridin- 1-yl]-2-[(2R,5R)-2- [[(2S)-2-[[3-[[(2S)-4- [[(2R,5R)-1-[2-[6-(4- fluorophenoxy)-3,3- dimethyl-2H- pyrrolo[3,2-b]pyridin- 1-yl]-2-oxo-ethyl]-5- methyl-piperazin-2- yl]methyl]morpholin-2- yl]methoxy]phenoxy] methyl]morpholin-4- yl]methyl]-5-methyl- piperazin-1- yl]ethanone |
| 143 | 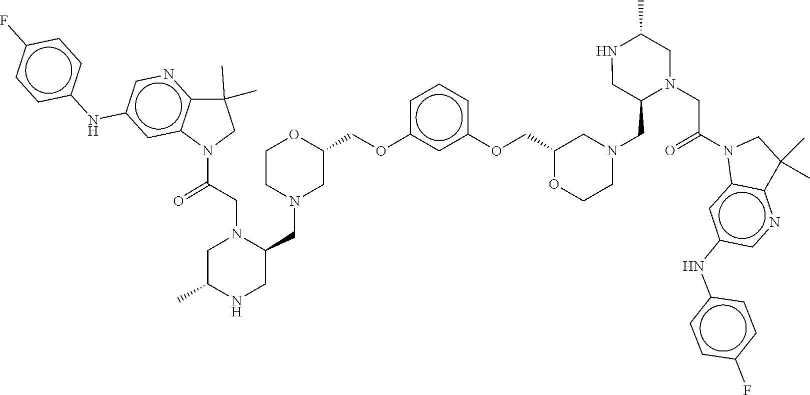 | -[6-(4-fluoroanilino)- 3,3-dimethyl-2H- pyrrolo[3,2-b]pyridin- 1-yl]-2-[(2R,5R)-2- [[(2S)-2-[[3-[[(2S)-4- [[(2R,5R)-1-[2-[6-(4- fluoroanilino)-3,3- dimethyl-2H- pyrrolo[3,2-b]pyridin- 1-yl]-2-oxo-ethyl]-5- methyl-piperazin-2- yl]methyl]morpholin-2- yl]methoxy]phenoxy] methyl]morpholin-4- yl]methyl]-5-methyl- piperazin-1- yl]ethenone |
| 144 | 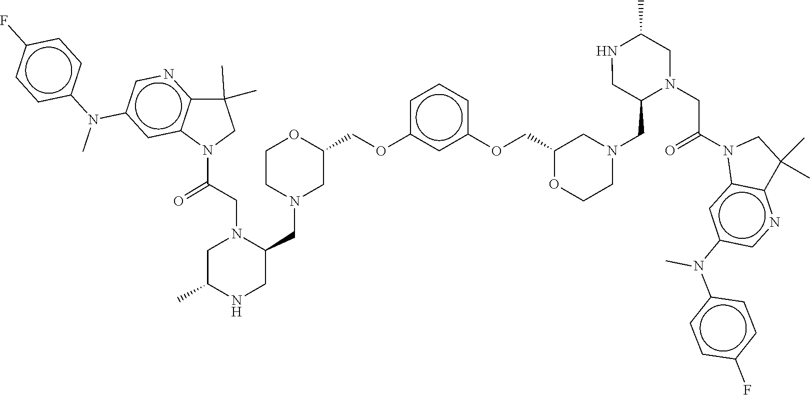 | 2,2′-((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1-diyl))bis(1-(6-((4- fluorophenyl)(methyl) amino)-3,3-dimethyl- 2,3-dihydro-1H- pyrrolo[3,2-b]pyridin- 1-yl)ethan-1-one) |
| 145 | 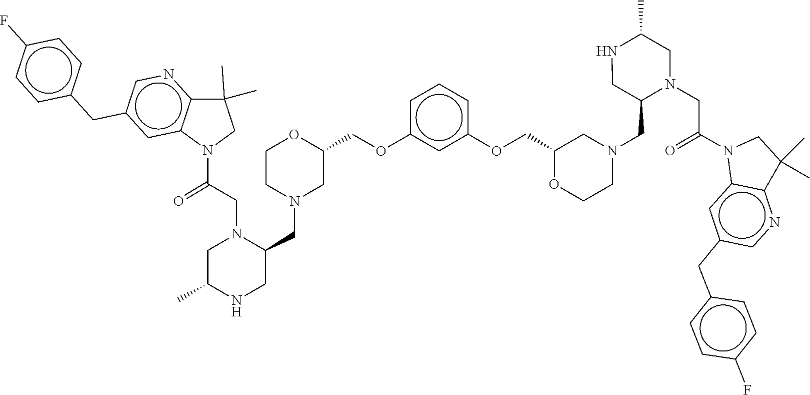 | 2,2′-((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1-diyl))bis(1-(6-(4- fluorobenzyl)-3,3- dimethyl-2,3-dihydro- 1H-pyrrolo[3,2- b]pyridin-1-yl)ethan-1- one) |
| 146 | 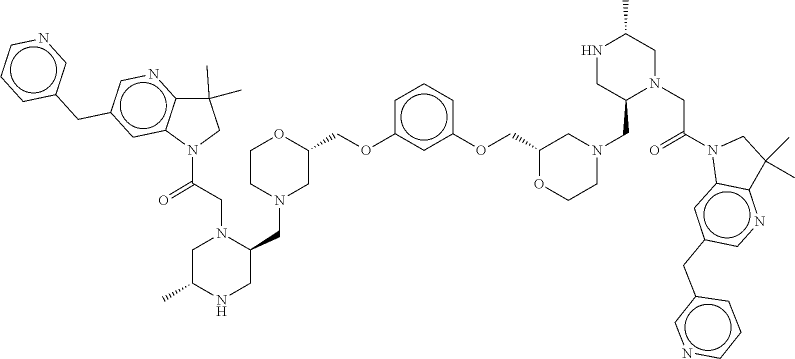 | 2,2′-((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1-diyl))bis(1-(3,3- dimethyl-6-(pyridin-3- ylmethyl)-2,3-dihydro- 1H-pyrrolo[3,2- b]pyridin-1-yl)ethan-1- one) |
| 147 | 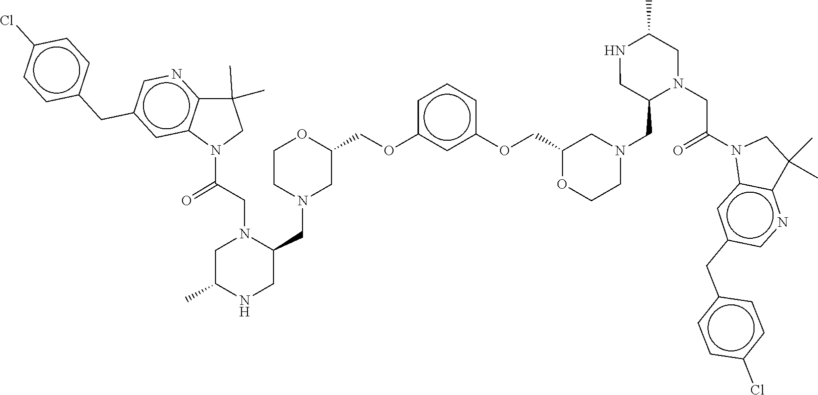 | 2,2′-((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1-diyl))bis(1-(6-(4- chlorobenzyl)-3,3- dimethyl-2,3-dihydro- 1H-pyrrolo[3,2- b]pyridin-1-yl)ethan-1- one) |
| 148 | 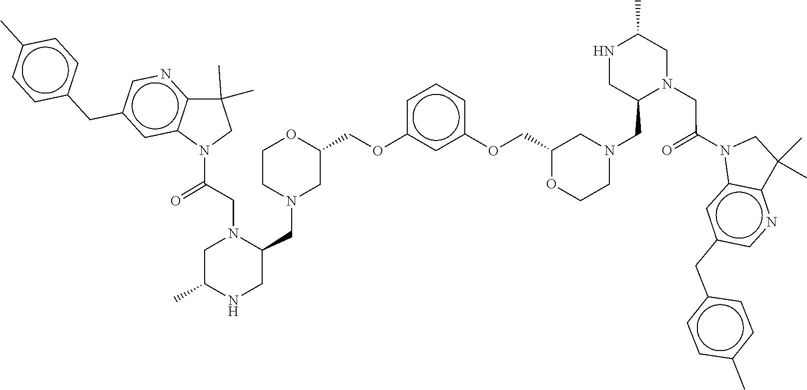 | 2,2′-((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1-diyl))bis(1-(3,3- dimethyl-6-(4- methylbenzyl)-2,3- dihydro-1H- pyrrolo[3,2-b]pyridin- 1-yl)ethan-1-one) |
| 149 | 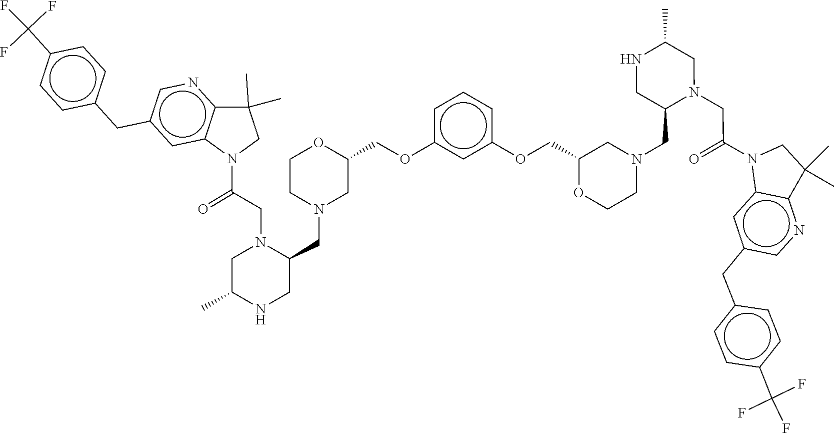 | 2,2′-((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1-diyl))bis(1-(3,3- dimethyl-6-(4- (trifluoromethyl)benzyl)- 2,3-dihydro-1H- pyrrolo[3,2-b]pyridin- 1-yl)ethan-1-one) |
| 150 | 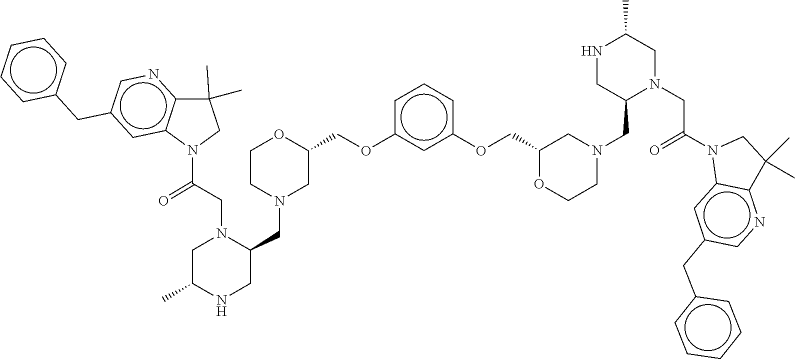 | 2,2′-((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1-diyl))bis(1-(6- benzyl-3,3-dimethyl- 2,3-dihydro-1H- pyrrolo[3,2-b]pyridin- 1-yl)ethan-1-one) |
| 151 | 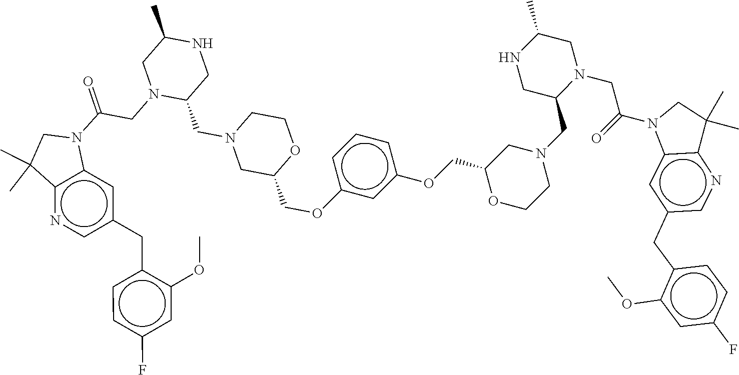 | 2,2′-((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1-diyl))bis(1-(6-(4- fluoro-2- methoxybenzyl)-3,3- dimethyl-2,3-dihydro- 1H-pyrrolo[3,2- b]pyridin-1-yl)ethan-1- one) |
| 152 | 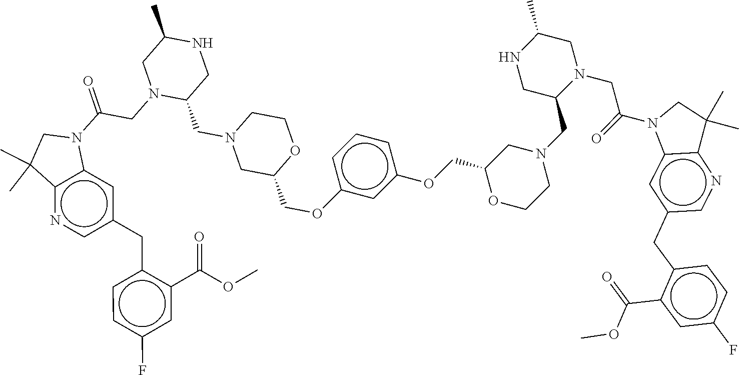 | dimethyl 6,6′-(((2,2′- ((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1- diyl))bis(acetyl))bis(3,3- dimethyl-2,3-dihydro- 1H-pyrrolo[3,2- b]pyridine-1,6- diyl))bis(methylene)) bis(3-fluorobenzoate) |
| 153 | 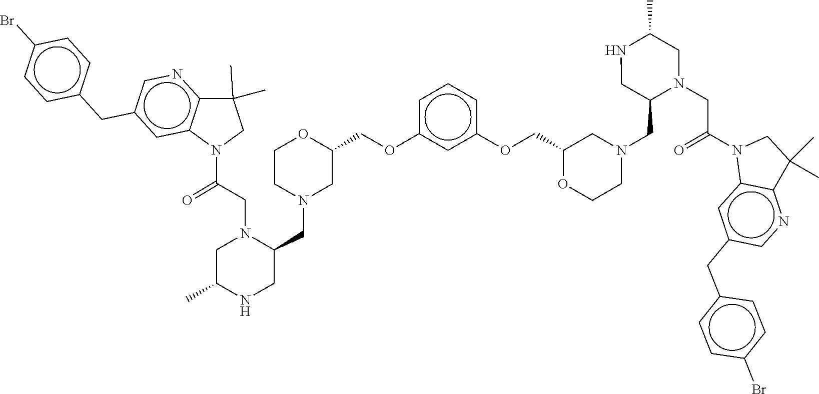 | 1-[6-[(4- bromophenyl)methyl]- 3,3-dimethyl-2H- pyrrolo[3,2-b]pyridin- 1-yl]-2-[(2R,5R)-2- [[(2S)-2-[3-[[(2S)-4- [[(2R,5R)-1-[2-[6-[(4- bromophenyl)methyl]- 3,3-dimethyl-2H- pyrrolo[3,2-b]pyridin- 1-yl]-2-oxo-ethyl]-5- methyl-piperazin-2- yl]methyl]morpholin-2- yl]methoxy]phenoxy] methyl]morpholin-4- yl]methyl]-5-methyl- piperazin-1- yl]ethanone |
| 154 | 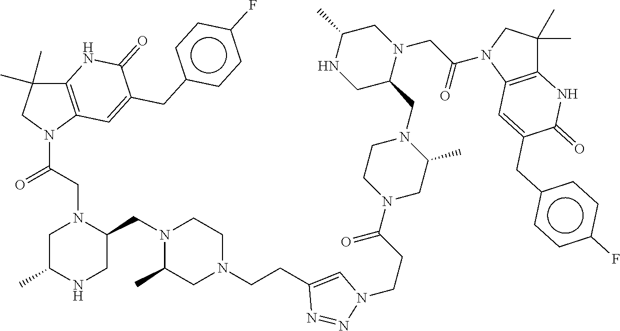 | 6-(4-fluorobenzyl)-1- (2-((2R,5R)-2-(((R)-4- (2-(1-(3-((R)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1-yl)- 3-oxopropyl)-1H-1,2,3- triazol-4-yl)ethyl)-2- methylpiperazin-1- yl)methyl)-5- methylpiperazin-1- yl)acetyl)-3,3- dimethyl-1,2,3,4- tetrahydro-5H- pyrrolo[3,2-b]pyridin- 5-one |
| 155 | 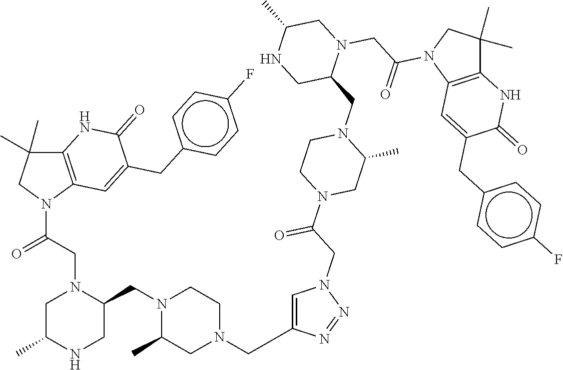 | 6-(4-fluorobenzyl)-1- (2-((2R,5R)-2-(((R)-4- ((1-(2-((R)-4-(((2R,5R)- 1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1-yl)- 2-oxoethyl)-1H-1,2,3- triazol-4-yl)methyl)-2- methylpiperazin-1- yl)methyl)-5- methylpiperazin-1- yl)acetyl)-3,3- dimethyl-1,2,3,4- tetrahydro-5H- pyrrolo[3,2-b]pyridin- 5-one |
| 156 | 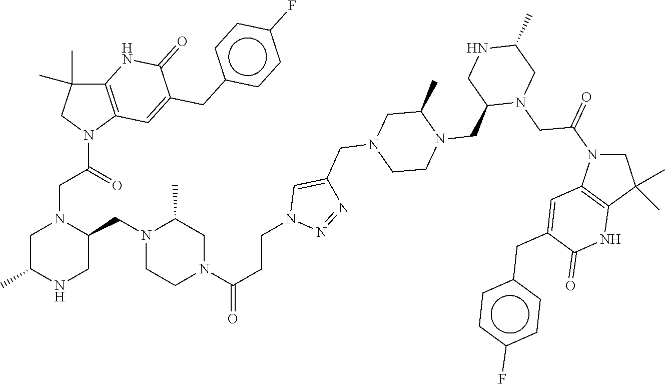 | 6-(4-fluorobenzyl)-1- (2-((2R,5R)-2-(((R)-4- ((1-(3-((R)-4-(((2R,5R)- 1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1-yl)- 3-oxopropyl)-1H-1,2,3- triazol-4-yl)methyl)-2- methylpiperazin-1- yl)methyl)-5- methylpiperazin-1- yl)acetyl)-3,3- dimethyl-1,2,3,4- tetrahydro-5H- pyrrolo[3,2-b]pyridin- 5-one |
| 157 | 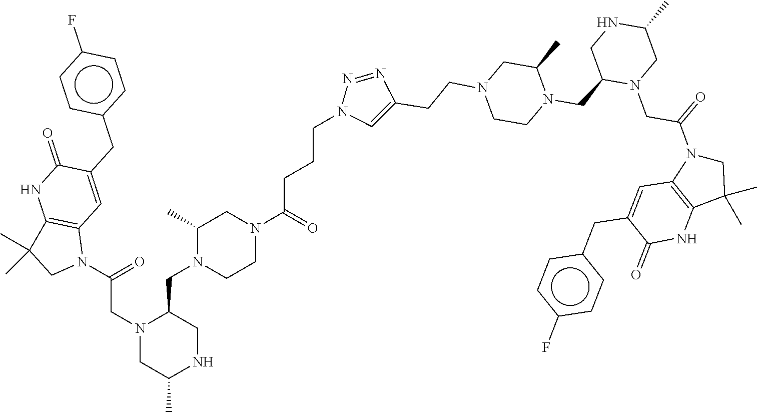 | 6-(4-fluorobenzyl)-1- (2-((2R,5R)-2-(((R)-4- (2-(1-(4-((R)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1-yl)- 4-oxobutyl)-1H-1,2,3- triazol-4-yl)ethyl)-2- methylpiperazin-1- yl)methyl)-5- methylpiperazin-1- yl)acetyl)-3,3- dimethyl-1,2,3,4- tetrahydro-5H- pyrrolo[3,2-b]pyridin- 5-one |
| 158 | 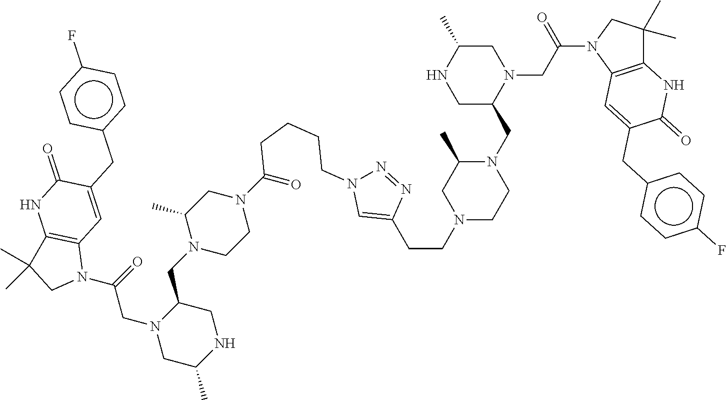 | 6-(4-fluorobenzyl)-1- (2-((2R,5R)-2-(((R)-4- (2-(1-(5-((R)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1-yl)- 5-oxopentyl)-1H-1,2,3- triazol-4-yl)ethyl)-2- methylpiperazin-1- yl)methyl)-5- methylpiperazin-1- yl)acetyl)-3,3- dimethyl-1,2,3,4- tetrahydro-5H- pyrrolo[3,2-b]pyridin- 5-one |
| 159 | 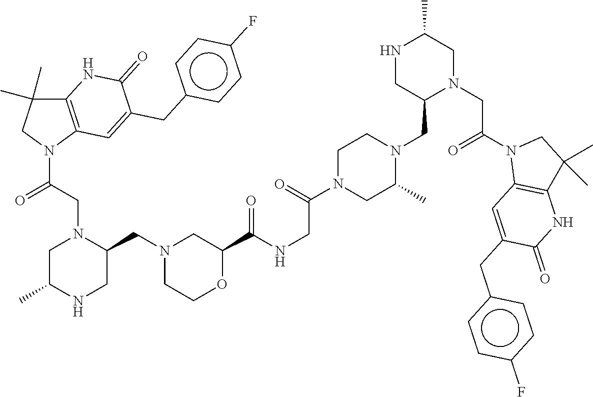 | (S)-4-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)-N-(2-((R)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1-yl)- 2- oxoethyl)morpholine- 2-carboxamide |
| 160 | 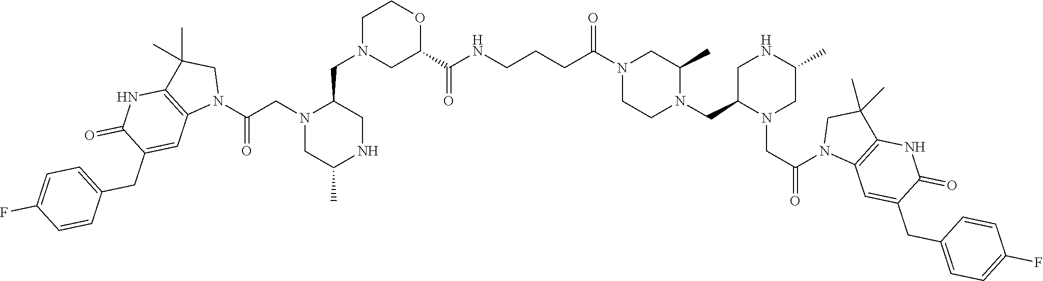 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCCC(=O)N3CCN(C[C@H]4CN[C@H](C)CN4CC(=O)N4CC(C)(C)c5[nH]c(=O)c(Cc6ccc(F)cc6)cc54)[C@H](C)C3)C2)CN1] | (S)-4-(((2R,5R)-1-(2- (6-(4-fluorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)-N-(4-((R)-4- (((2R,5R)-1-(2-(6-(4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)-3- methylpiperazin-1-yl)- 4- oxobutyl)morpholine- 2-carboxamide |
| 161 | 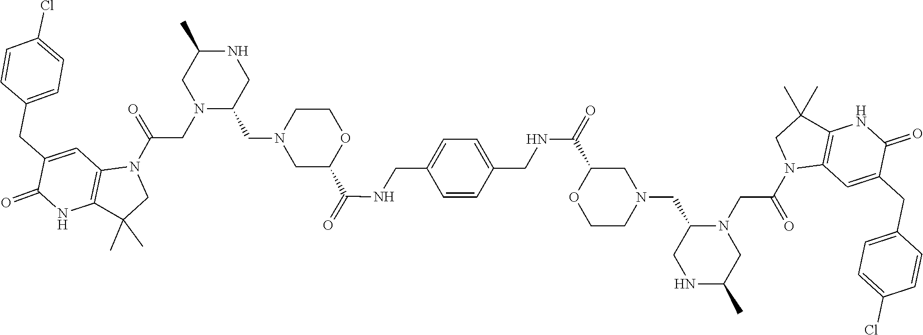 [SMILES: C[C@@H]1CN(CC(=O)N2CC(C)(C)c3[nH]c(=O)c(Cc4ccc(Cl)cc4)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(Cl)cc7)cc65)CCO4)cc3)C2)CN1] | (2S,2′S)-N,N′-(1,4- phenylenebis(methylene)) bis(4-(((2R,5R)-1- (2-(6-(4-chlorobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 162 | 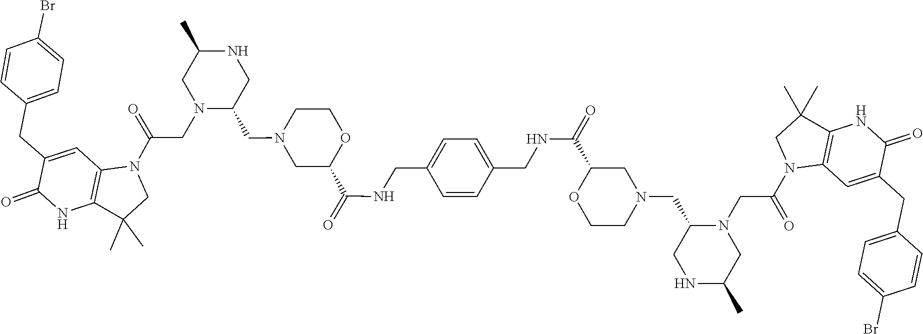 [SMILES: C[C@@H]1CN(CC(=O)N2CC(C)(C)c3[nH]c(=O)c(Cc4ccc(Br)cc4)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(Br)cc7)cc65)CCO4)cc3)C2)CN1] | (2S,2′S)-N,N′-(1,4- phenylenebis(methylene)) bis(4-(((2R,5R)-1- (2-(6-(4-bromobenzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 163 | 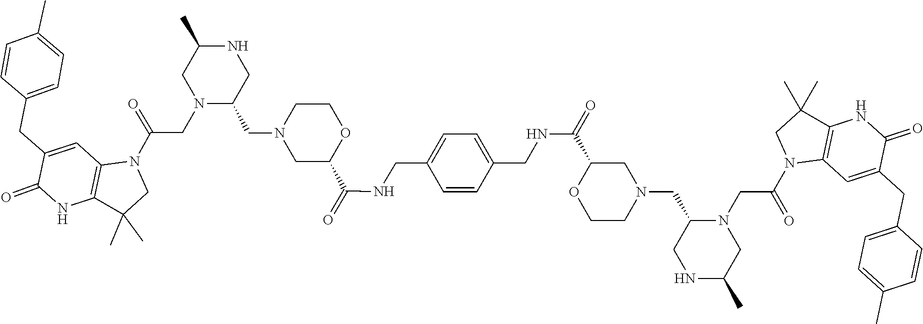 [SMILES: Cc1ccc(Cc2cc3c([nH]c2=O)C(C)(C)CN3C(=O)CN2C[C@@H](C)NC[C@@H]2CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(C)cc7)cc65)CCO4)cc3)C2)cc1] | (2S,2′S)-N,N′-(1,4- phenylenebis(methylen e))bis(4-(((2R,5R)-1- (2-(3,3-dimethyl-6-(4- methylbenzyl)-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 164 | 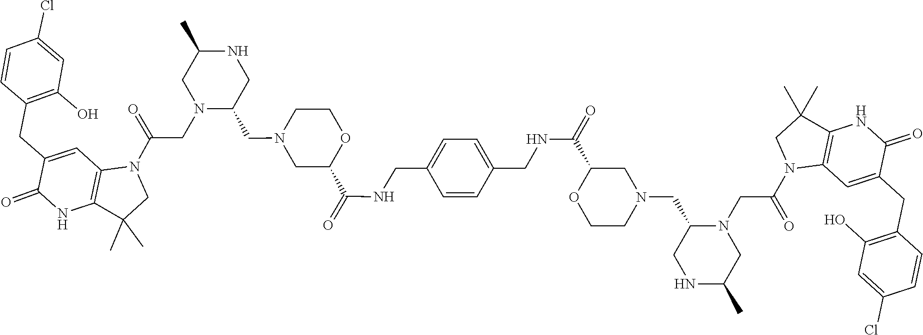 [SMILES: C[C@@H]1CN(CC(=O)N2CC(C)(C)c3[nH]c(=O)c(Cc4ccc(Cl)cc4O)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(Cl)cc7O)cc65)CCO4)cc3)C2)CN1] | (2S,2′S)-N,N′-(1,4- phenylenebis(methylene)) bis(4-(((2R,5R)-1- (2-(6-(4-chloro-2- hydroxybenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 165 | 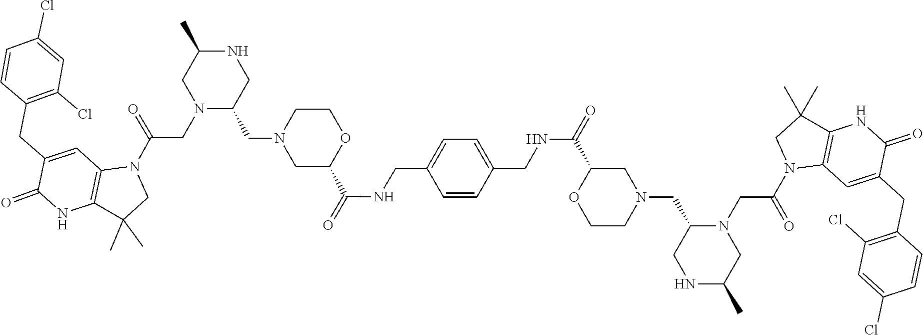 [SMILES: C[C@@H]1CN(CC(=O)N2CC(C)(C)c3[nH]c(=O)c(Cc4ccc(Cl)cc4Cl)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(Cl)cc7Cl)cc65)CCO4)cc3)C2)CN1] | (2S,2′S)-N,N′-(1,4- phenylenebis(methylene)) bis(4-(((2R,5R)-1- (2-(6-(2,4- dichlorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 166 | 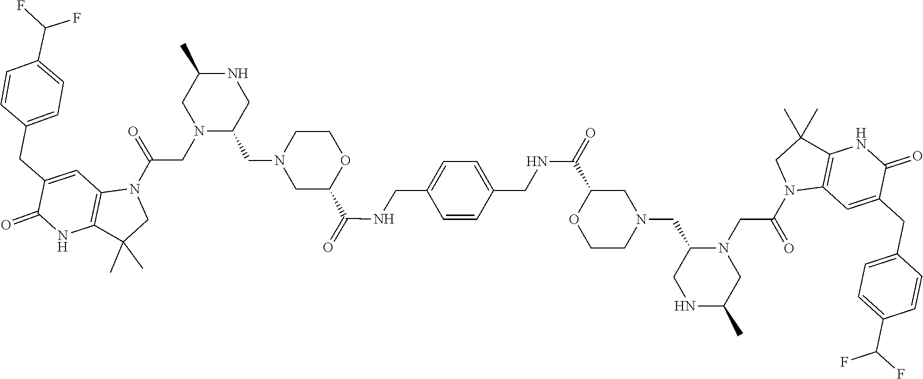 [SMILES: C[C@@H]1CN(CC(=O)N2CC(C)(C)c3[nH]c(=O)c(Cc4ccc(C(F)F)cc4)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(C(F)F)cc7)cc65)CCO4)cc3)C2)CN1] | (2S,2′S)-N,N′-(1,4- phenylenebis(methylene)) bis(4-(((2R,5R)-1- (2-(6-(4- (difluoromethyl)benzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 167 | 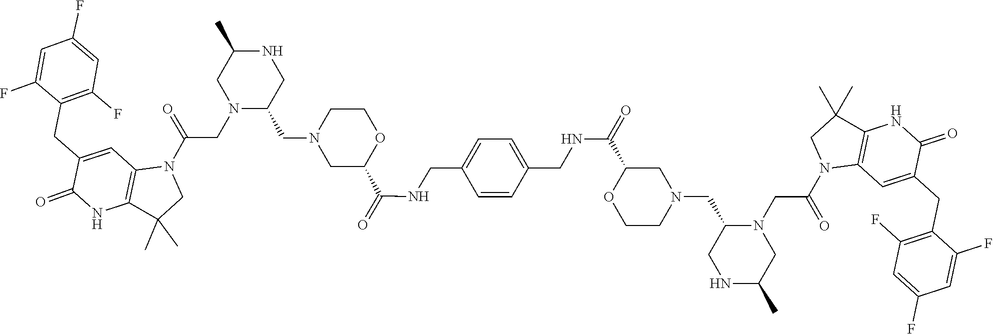 [SMILES: C[C@@H]1CN(CC(=O)N2CC(C)(C)c3[nH]c(=O)c(Cc4c(F)cc(F)cc4F)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7c(F)cc(F)cc7F)cc65)CCO4)cc3)C2)CN1] | (2S,2′S)-N,N′-(1,4- phenylenebis(methylene)) bis(4-(((2R,5R)-1- (2-(3,3-dimethyl-5- oxo-6-(2,4,6- trifluorobenzyl)- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 168 | 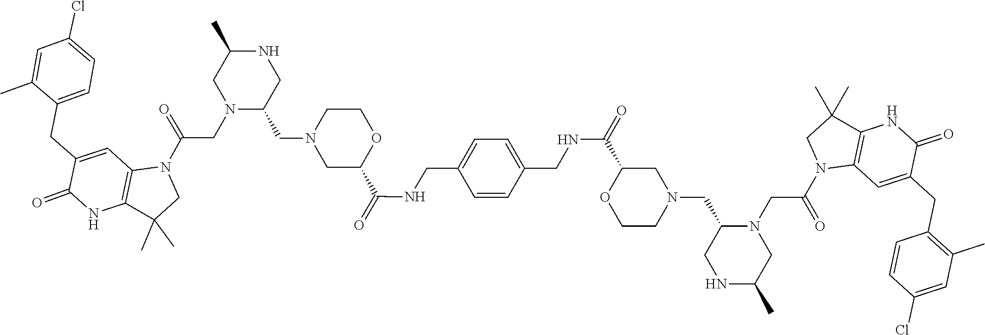 | (2S,2′S)-N,N′-(1,4- phenylenebis(methylene)) bis(4-(((2R,5R)-1- (2-(6-(4-chloro-2- methylbenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 169 | 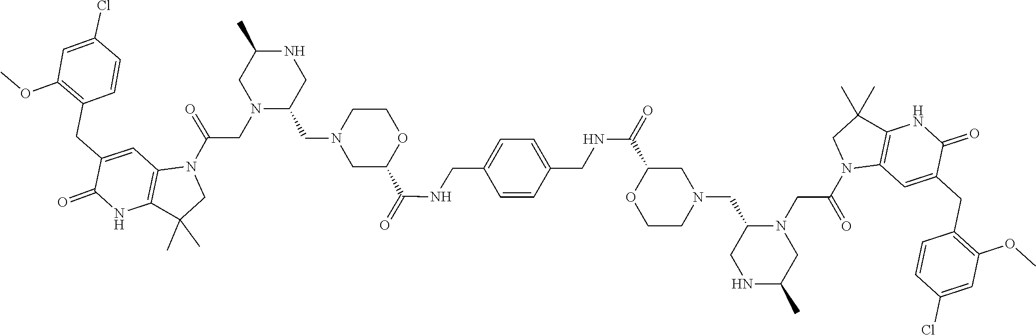 | (2S,2′S)-N,N′-(1,4- phenylenebis(methylene)) bis(4-(((2R,5R)-1- (2-(6-(4-chloro-2- methoxybenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 170 | 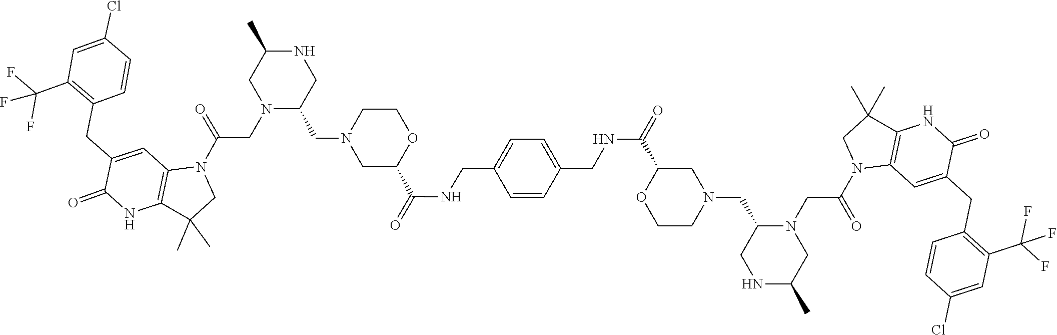 [SMILES: C[C@@H]1CN(CC(=O)N2CC(C)(C)c3[nH]c(=O)c(Cc4ccc(Cl)cc4C(F)(F)F)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(Cl)cc7C(F)(F)F)cc65)CCO4)cc3)C2)CN1] | (2S,2′S)-N,N′-(1,4- phenylenebis(methylene)) bis(4-(((2R,5R)-1- (2-(6-(4-chloro-2- (trifluoromethyl)benzyl)- 3,3-dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 171 | 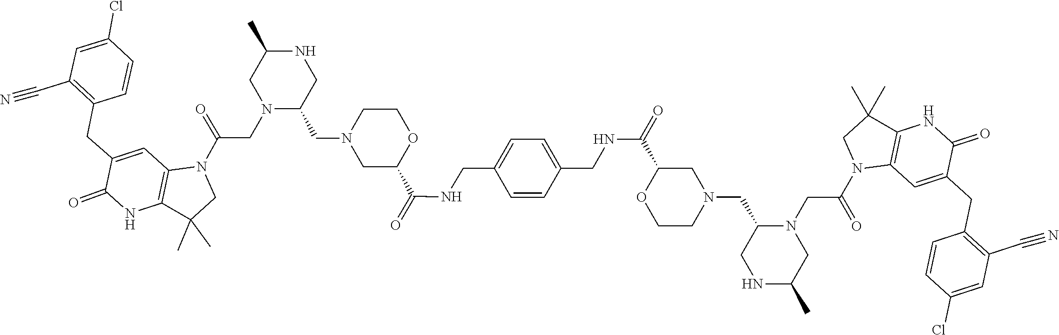 [SMILES: C[C@@H]1CN(CC(=O)N2CC(C)(C)c3[nH]c(=O)c(Cc4ccc(Cl)cc4C#N)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(Cl)cc7C#N)cc65)CCO4)cc3)C2)CN1] | (2S,2′S)-N,N′-(1,4- phenylenebis(methylene)) bis(4-(((2R,5R)-1- (2-(6-(4-chloro-2- cyanobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 172 | 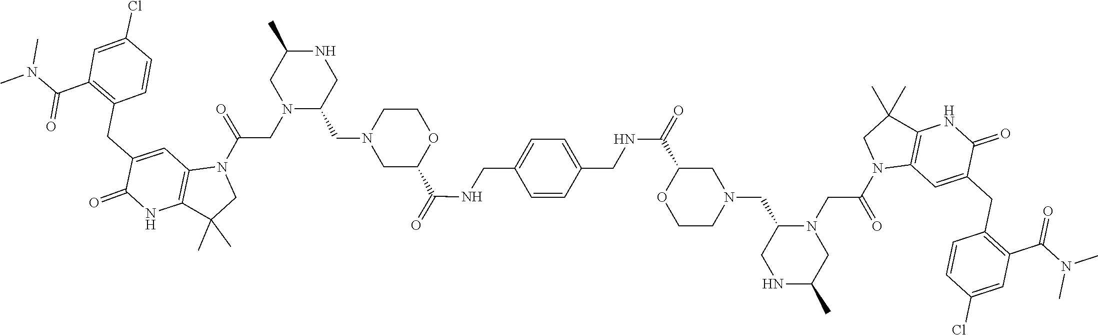 | (2S,2′S)-N,N′-(1,4- phenylenebis(methylene)) bis(4-(((2R,5R)-1- (2-(6-(4-chloro-2- (dimethylcarbamoyl) benzyl)-3,3-dimethyl-5- oxo-2,3,4,5-tetrahydro- 1H-pyrrolo[3,2- b]pyridin-1-yl)-2- oxoethyl)-5- methylpiperazin-2- yl)methyl)morpholine- 2-carboxamide) |
| 173 | 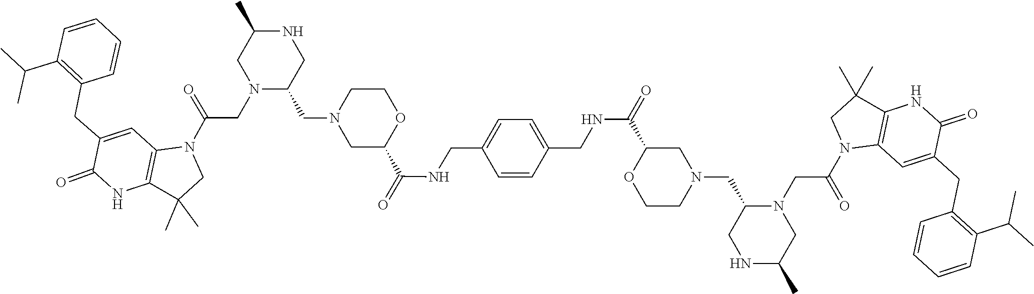 [SMILES: CC(C)c1ccccc1Cc1cc2c([nH]c1=O)C(C)(C)CN2C(=O)CN1C[C@@H](C)NC[C@@H]1CN1CCO[C@H](C(=O)NCc2ccc(CNC(=O)[C@@H]3CN(C[C@H]4CN[C@H](C)CN4CC(=O)N4CC(C)(C)c5[nH]c(=O)c(Cc6ccccc6C(C)C)cc54)CCO3)cc2)C1] | (2S,2′S)-N,N′-(1,4- phenylenebis(methylene)) bis(4-(((2R,5R)-1- (2-(6-(2- isopropylbenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 174 | 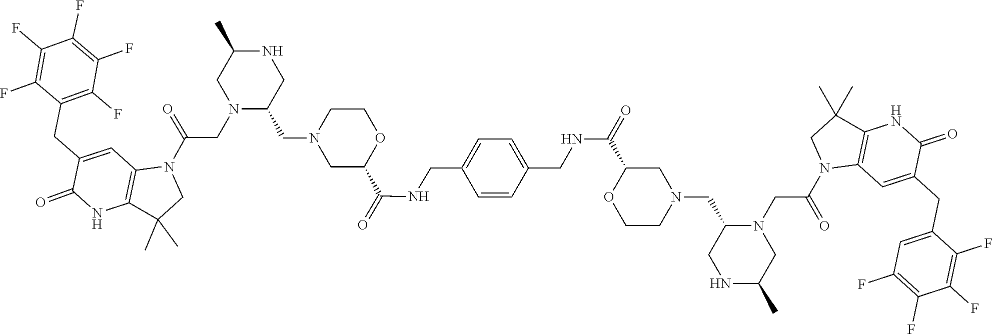 [SMILES: C[C@@H]1CN(CC(=O)N2CC(C)(C)c3[nH]c(=O)c(Cc4cc(F)c(F)c(F)c4F)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7c(F)c(F)c(F)c(F)c7F)cc65)CCO4)cc3)C2)CN1] | (2S,2′S)-N,N′-(1,4- phenylenebis(methylene)) bis(4-(((2R,5R)-1- (2-(3,3-dimethyl-5- oxo-6-(2,3,4,5- tetrafluorobenzyl)- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 175 | 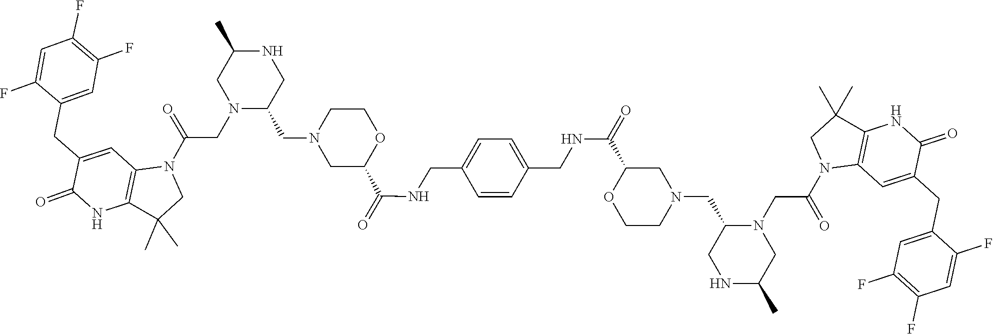 [SMILES: C[C@@H]1CN(CC(=O)N2CC(C)(C)c3[nH]c(=O)c(Cc4cc(F)c(F)cc4F)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7cc(F)c(F)cc7F)cc65)CCO4)cc3)C2)CN1] | (2S,2′S)-N,N′-(1,4- phenylenebis(methylene)) bis(4-(((2R,5R)-1- (2-(3,3-dimethyl-5- oxo-6-(2,4,5- trifluorobenzyl)- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 176 | 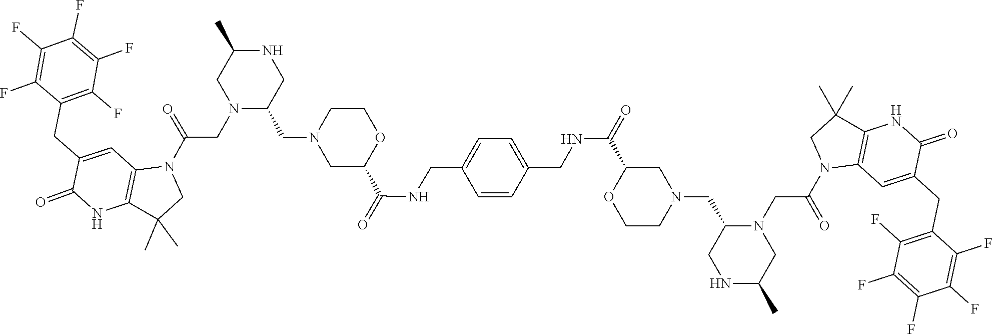 [SMILES: C[C@@H]1CN(CC(=O)N2CC(C)(C)c3[nH]c(=O)c(Cc4c(F)c(F)c(F)c(F)c4F)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7c(F)c(F)c(F)c(F)c7F)cc65)CCO4)cc3)C2)CN1] | (2S,2′S)-N,N′-(1,4- phenylenebis(methylene)) bis(4-(((2R,5R)-1- (2-(3,3-dimethyl-5- oxo-6- ((perfluorophenyl)methyl)- 2,3,4,5-tetrahydro- 1H-pyrrolo[3,2- b]pyridin-1-yl)-2- oxoethyl)-5- methylpiperazin-2- yl)methyl)morpholine- 2-carboxamide) |
| 177 | 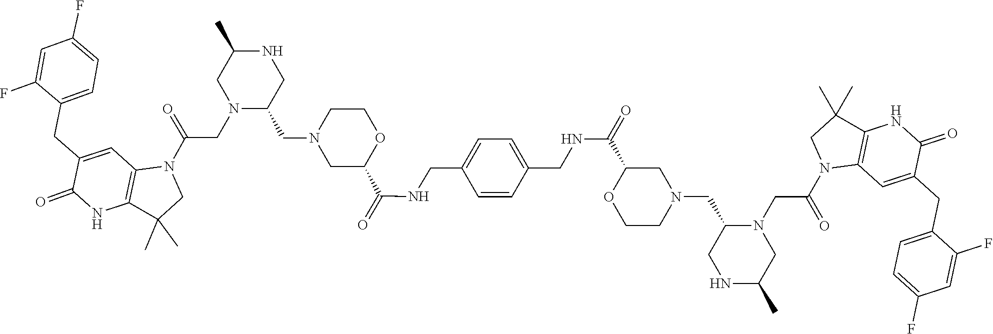 [SMILES: C[C@@H]1CN(CC(=O)N2CC(C)(C)c3[nH]c(=O)c(Cc4ccc(F)cc4F)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(F)cc7F)cc65)CCO4)cc3)C2)CN1] | (2S,2′S)-N,N′-(1,4- phenylenebis(methylene)) bis(4-(((2R,5R)-1- (2-(6-(2,4- difluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 178 | 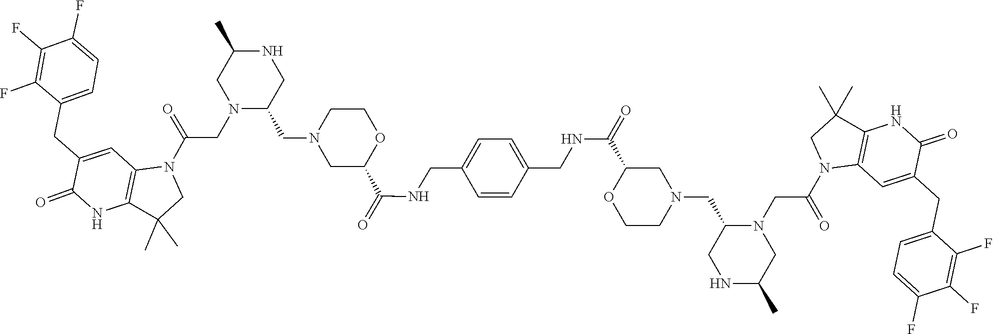 [SMILES: C[C@@H]1CN(CC(=O)N2CC(C)(C)c3[nH]c(=O)c(Cc4ccc(F)c(F)c4F)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(F)c(F)c7F)cc65)CCO4)cc3)C2)CN1] | (2S,2′S)-N,N′-(1,4- phenylenebis(methylene)) bis(4-(((2R,5R)-1- (2-(3,3-dimethyl-5- oxo-6-(2,3,4- trifluorobenzyl)- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 179 | 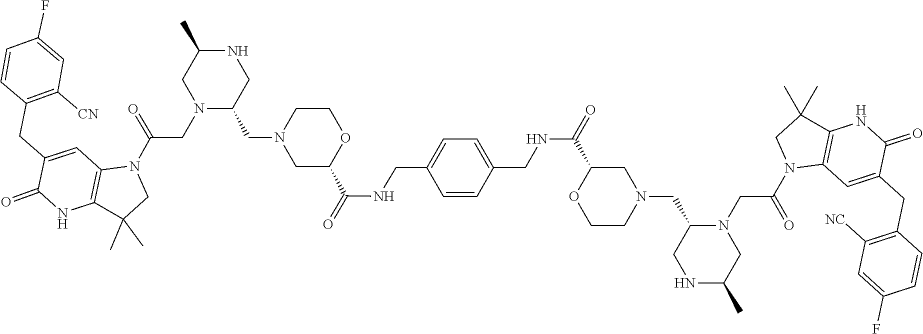 [SMILES: C[C@@H]1CN(CC(=O)N2CC(C)(C)c3[nH]c(=O)c(Cc4ccc(F)cc4C#N)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(F)cc7C#N)cc65)CCO4)cc3)C2)CN1] | (2S,2′S)-N,N′-(1,4- phenylenebis(methylene)) bis(4-(((2R,5R)-1- (2-(6-(2-cyano-4- fluorobenzyl)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 180 | 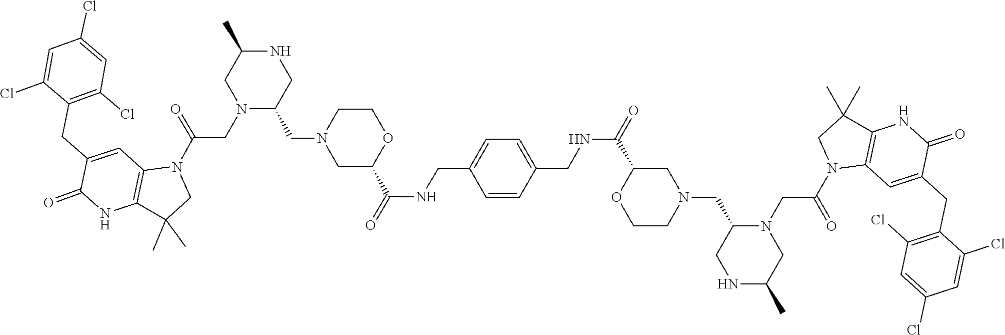 [SMILES: C[C@@H]1CN(CC(=O)N2CC(C)(C)c3[nH]c(=O)c(Cc4c(Cl)cc(Cl)cc4Cl)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7c(Cl)cc(Cl)cc7Cl)cc65)CCO4)cc3)C2)CN1] | (2S,2′S)-N,N′-(1,4- phenylenebis(methylene)) bis(4-(((2R,5R)-1- (2-(3,3-dimethyl-5- oxo-6-(2,4,6- trichlorobenzyl)- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 181 | 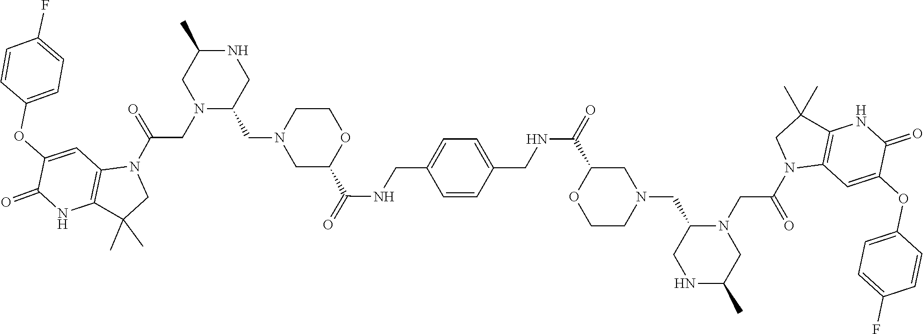 [SMILES: C[C@@H]1CN(CC(=O)N2CC(C)(C)c3[nH]c(=O)c(Oc4ccc(F)cc4)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Oc7ccc(F)cc7)cc65)CCO4)cc3)C2)CN1] | (2S,2′S)-N,N′-(1,4- phenylenebis(methylene)) bis(4-(((2R,5R)-1- (2-(6-(4- fluorophenoxy)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 182 | 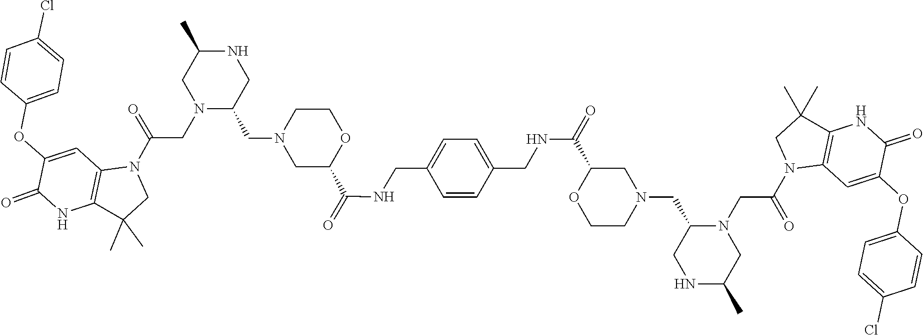 [SMILES: C[C@@H]1CN(CC(=O)N2CC(C)(C)c3[nH]c(=O)c(Oc4ccc(Cl)cc4)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Oc7ccc(Cl)cc7)cc65)CCO4)cc3)C2)CN1] | (2S,2′S)-N,N′-(1,4- phenylenebis(methylene)) bis(4-(((2R,5R)-1- (2-(6-(4- chlorophenoxy)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 183 | 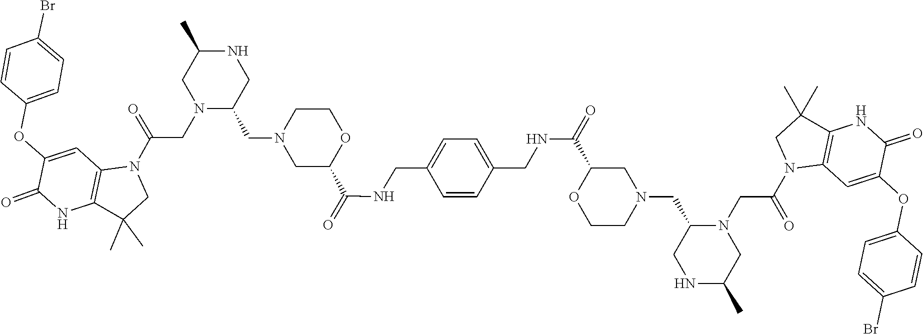 [SMILES: C[C@@H]1CN(CC(=O)N2CC(C)(C)c3[nH]c(=O)c(Oc4ccc(Br)cc4)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Oc7ccc(Br)cc7)cc65)CCO4)cc3)C2)CN1] | (2S,2′S)-N,N′-(1,4- phenylenebis(methylene)) bis(4-(((2R,5R)-1- (2-(6-(4- bromophenoxy)-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 184 | 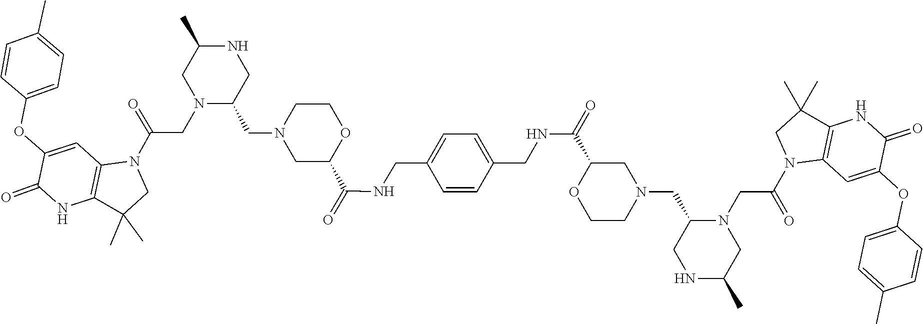 [SMILES: Cc1ccc(Oc2cc3c([nH]c2=O)C(C)(C)CN3C(=O)CN2C[C@@H](C)NC[C@@H]2CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Oc7ccc(C)cc7)cc65)CCO4)cc3)C2)cc1] | (2S,2′S)-N,N′-(1,4- phenylenebis(methylene)) bis(4-(((2R,5R)-1- (2-(3,3-dimethyl-5- oxo-6-(p-tolyloxy)- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 185 | 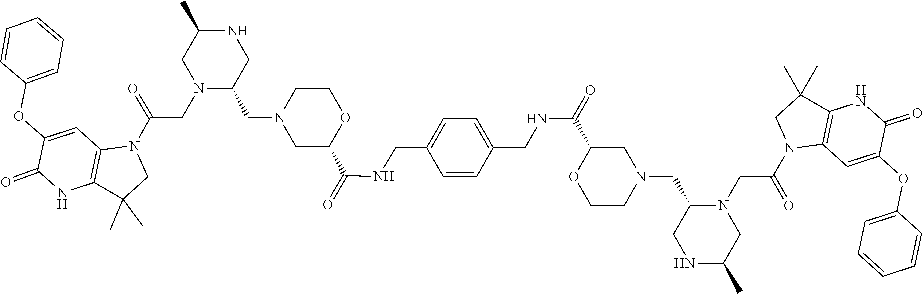 [SMILES: C[C@@H]1CN(CC(=O)N2CC(C)(C)c3[nH]c(=O)c(Oc4ccccc4)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Oc7ccccc7)cc65)CCO4)cc3)C2)CN1] | (2S,2′S)-N,N′-(1,4- phenylenebis(methylene)) bis(4-(((2R,5R)-1- (2-(3,3-dimethyl-5- oxo-6-phenoxy-2,3,4,5- tetrahydro-1H- pyrrolo[3,2-b]pyridin- 1-yl)-2-oxoethyl)-5- methylpiperazin-2- yl)methyl)morpholine- 2-carboxamide) |
| 186 | 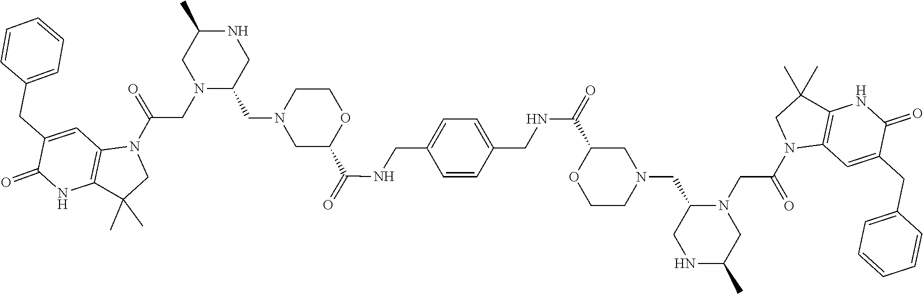 [SMILES: C[C@@H]1CN(CC(=O)N2CC(C)(C)c3[nH]c(=O)c(Cc4ccccc4)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccccc7)cc65)CCO4)cc3)C2)CN1] | (2S,2′S)-N,N′-(1,4- phenylenebis(methylene)) bis(4-(((2R,5R)-1- (2-(6-benzyl-3,3- dimethyl-5-oxo- 2,3,4,5-tetrahydro-1H- pyrrolo[3,2-b]pyridin- 1-yl)-2-oxoethyl)-5- methylpiperazin-2- yl)methyl)morpholine- 2-carboxamide) |
| 187 | 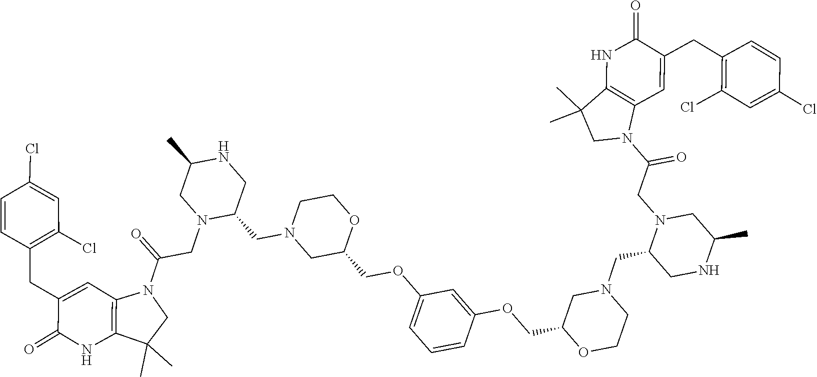 [SMILES: C[C@@H]1CN(CC(=O)N2CC(C)(C)c3[nH]c(=O)c(Cc4ccc(Cl)cc4Cl)cc32)[C@@H](CN2CCO[C@H](COc3cccc(OC[C@@H]4CN(C[C@H]5CN[C@H](C)CN5CC(=O)N5CC(C)(C)c6[nH]c(=O)c(Cc7ccc(Cl)cc7Cl)cc65)CCO4)c3)C2)CN1] | 1,1′-(2,2′- ((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1- diyl))bis(acetyl))bis(6- (2,4-dichlorobenzyl)- 3,3-dimethyl-1,2,3,4- tetrahydro-5H- pyrrolo[3,2-b]pyridin- 5-one) |
| 188 | 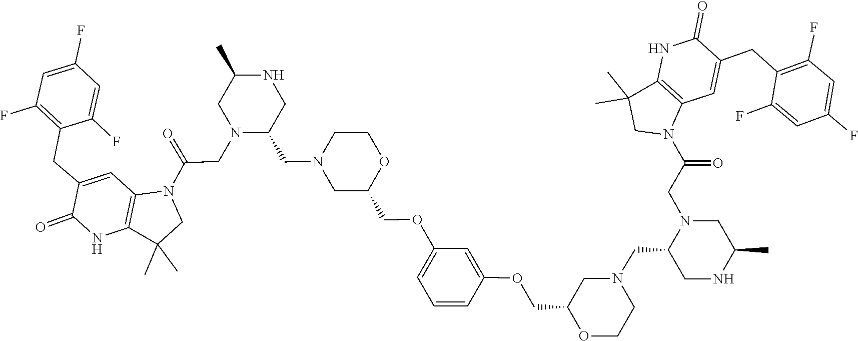 [SMILES: C[C@@H]1CN(CC(=O)N2CC(C)(C)c3[nH]c(=O)c(Cc4c(F)cc(F)cc4F)cc32)[C@@H](CN2CCO[C@H](COc3cccc(OC[C@@H]4CN(C[C@H]5CN[C@H](C)CN5CC(=O)N5CC(C)(C)c6[nH]c(=O)c(Cc7c(F)cc(F)cc7F)cc65)CCO4)c3)C2)CN1] | 1,1′-(2,2′- ((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1- diyl))bis(acetyl))bis(3,3- dimethyl-6-(2,4,6- trifluorobenzyl)- 1,2,3,4-tetrahydro-5H- pyrrolo[3,2-b]pyridin- 5-one) |
| 189 | 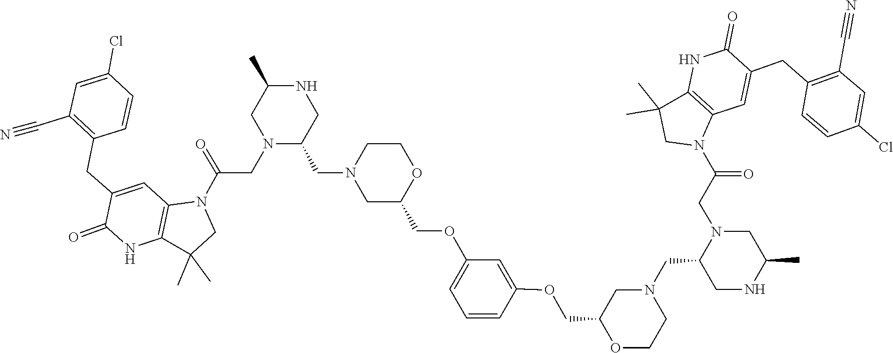 [SMILES: C[C@@H]1CN(CC(=O)N2CC(C)(C)c3[nH]c(=O)c(Cc4ccc(Cl)cc4C#N)cc32)[C@@H](CN2CCO[C@H](COc3cccc(OC[C@@H]4CN(C[C@H]5CN[C@H](C)CN5CC(=O)N5CC(C)(C)c6[nH]c(=O)c(Cc7ccc(Cl)cc7C#N)cc65)CCO4)c3)C2)CN1] | 6,6′-(((2,2′- ((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1- diyl))bis(acetyl))bis(3,3- dimethyl-5-oxo- 2,3,4,5-tetrahydro-1H- pyrrolo[3,2-b]pyridine- 1,6- diyl))bis(methylene)) bis(3-chlorobenzonitrile) |
| 190 | 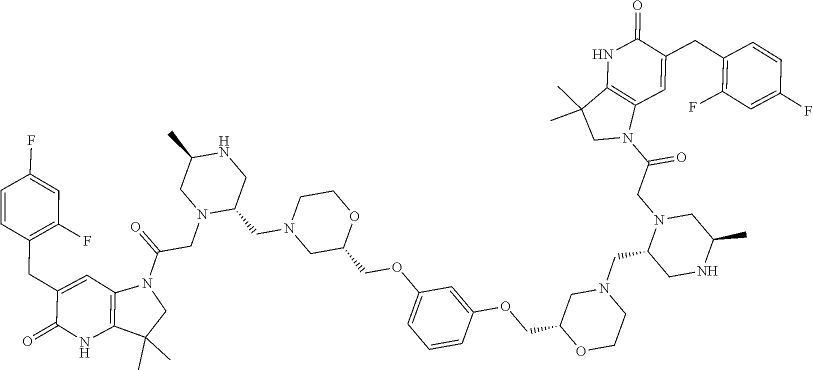 [SMILES: C[C@@H]1CN(CC(=O)N2CC(C)(C)c3[nH]c(=O)c(Cc4ccc(F)cc4F)cc32)[C@@H](CN2CCO[C@H](COc3cccc(OC[C@@H]4CN(C[C@H]5CN[C@H](C)CN5CC(=O)N5CC(C)(C)c6[nH]c(=O)c(Cc7ccc(F)cc7F)cc65)CCO4)c3)C2)CN1] | 1,1′-(2,2′- ((3R,3′R,6R,6′R)- (((2S,2′S)-((1,3- phenylenebis(oxy))bis (methylene))bis (morpholine-2,4- diyl))bis(methylene)) bis(3-methylpiperazine- 6,1- diyl))bis(acetyl))bis(6- (2,4-difluorobenzyl)- 3,3-dimethyl-1,2,3,4- tetrahydro-5H- pyrrolo[3,2-b]pyridin- 5-one) |

Pharmaceutically acceptable salts are also within the scope of the invention with respect to all compounds 1-190 set forth herein. In some embodiments, each of compounds 1-190 may be present generically as hydrochloride (i.e., HCl salts), e.g., more specifically a dihydrochloride, (2 HCl) salt. In some embodiments, the compounds 1-190 may be present generically as the mono, di, or mixture of mono and di-acid acetate, trifluoroacetate, citrate salt forms. In some embodiments, the salt form of compounds 1-190 may be a blend of the hydrohalide and dihydrohalide (i.e., the mono and di-acid) forms. Also, within the scope of the invention are any of compounds 1-190 present as a single species, including pharmaceutically acceptable salts thereof, as well as any of these compounds in free base form.

Specific examples of linkers (L) that can be used in accordance with the present invention include those selected from the group consisting of:

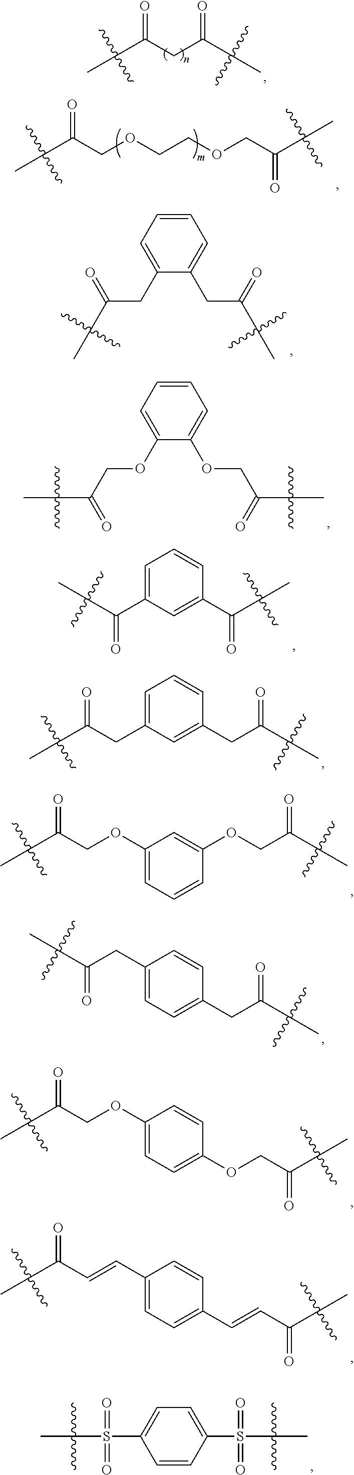

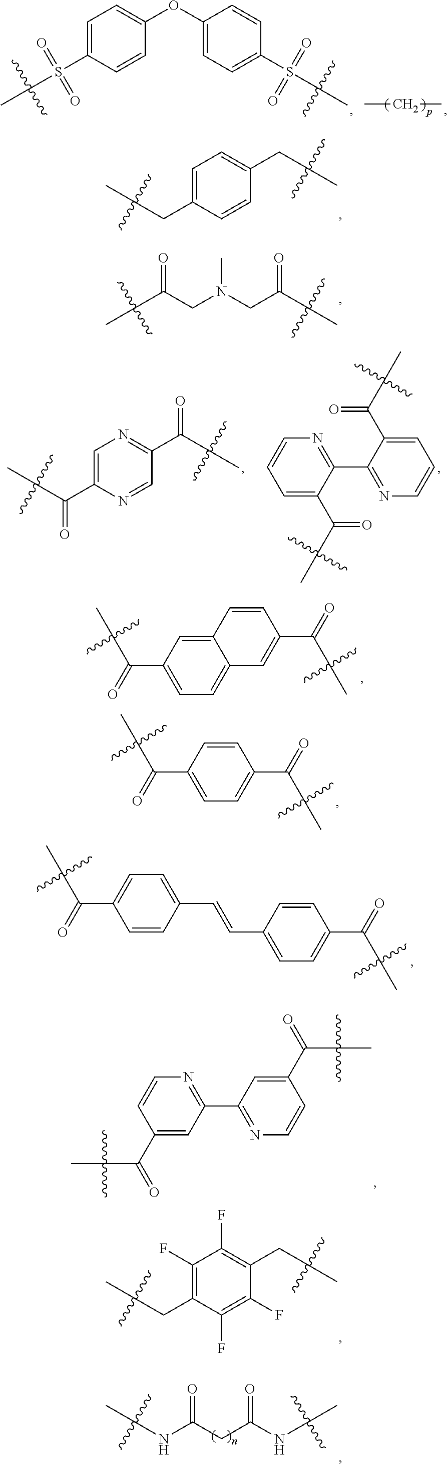

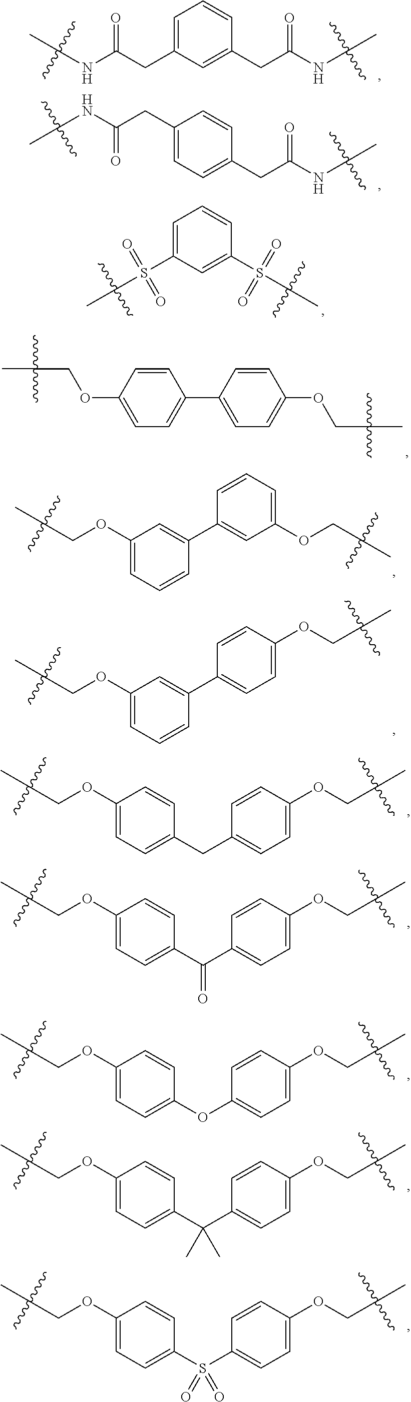

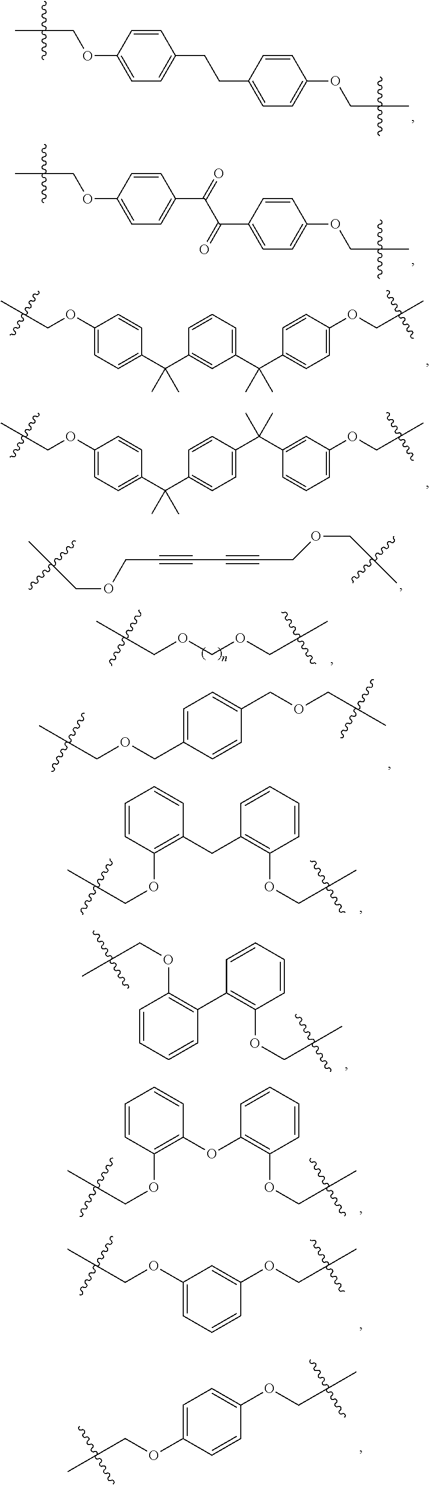

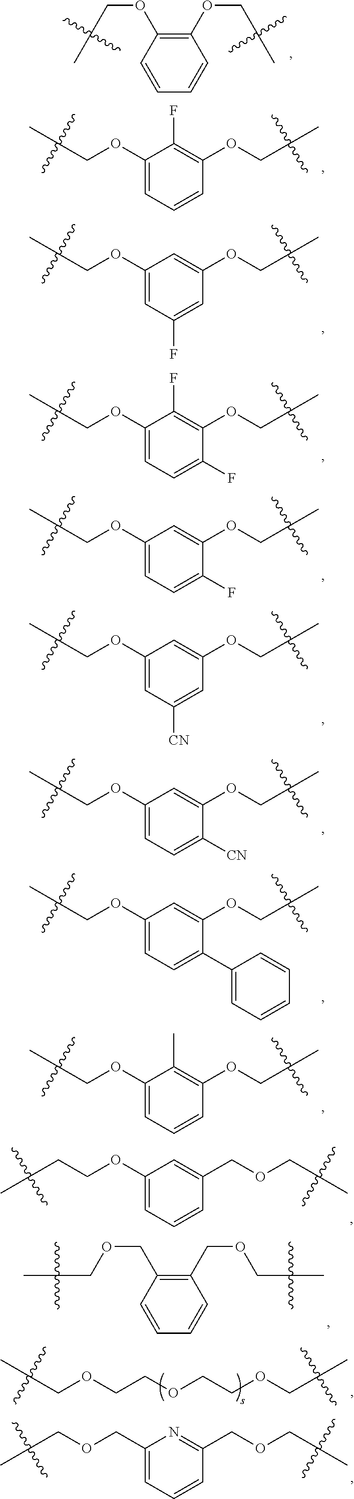

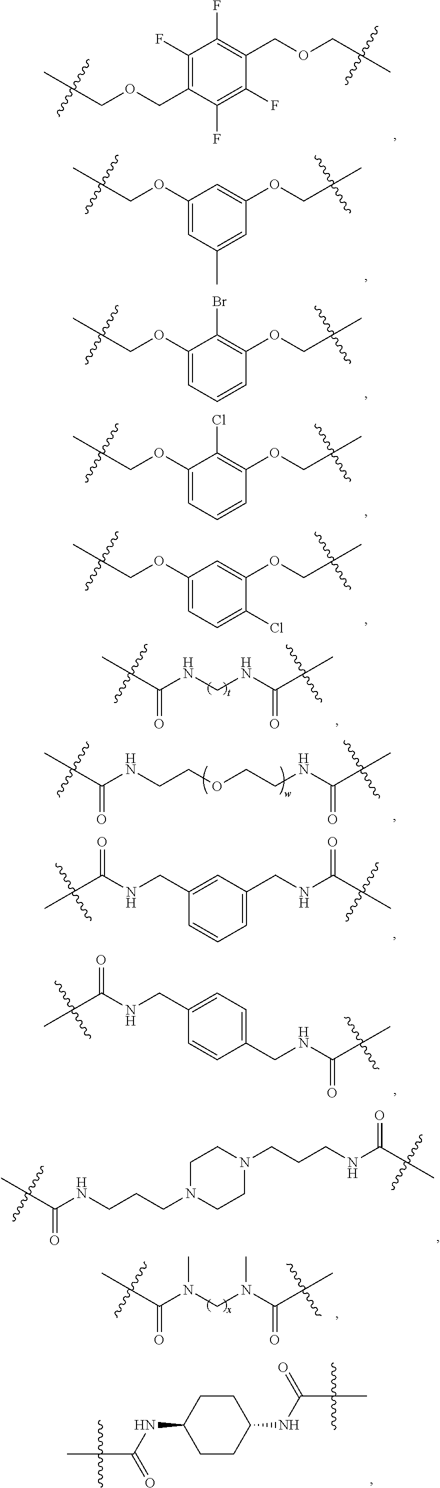

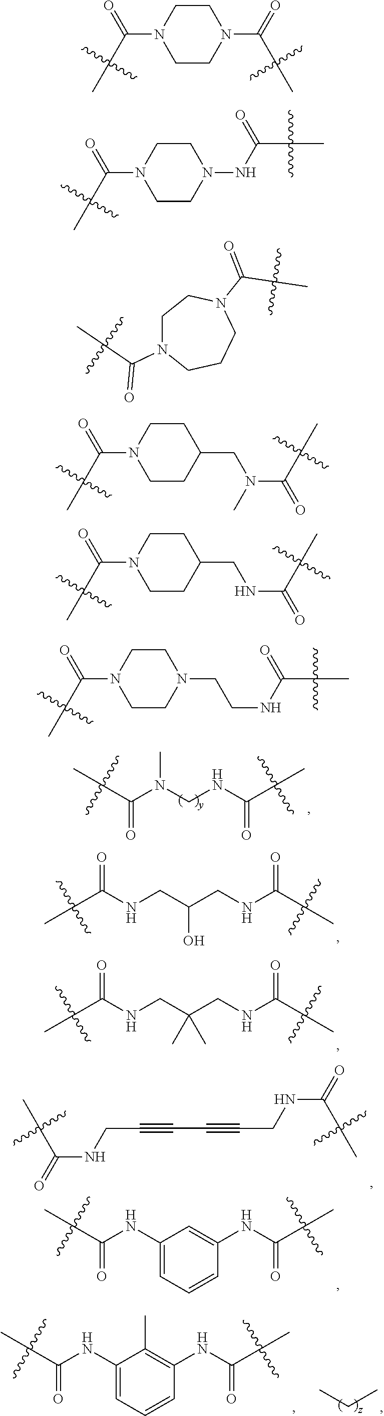

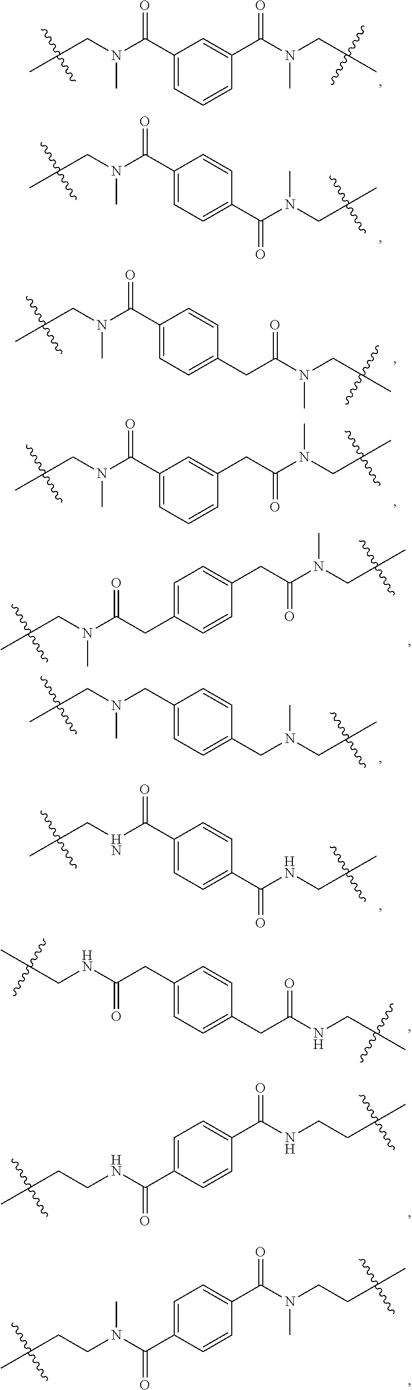

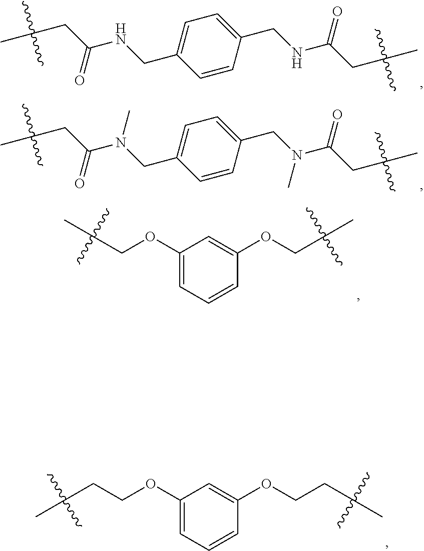

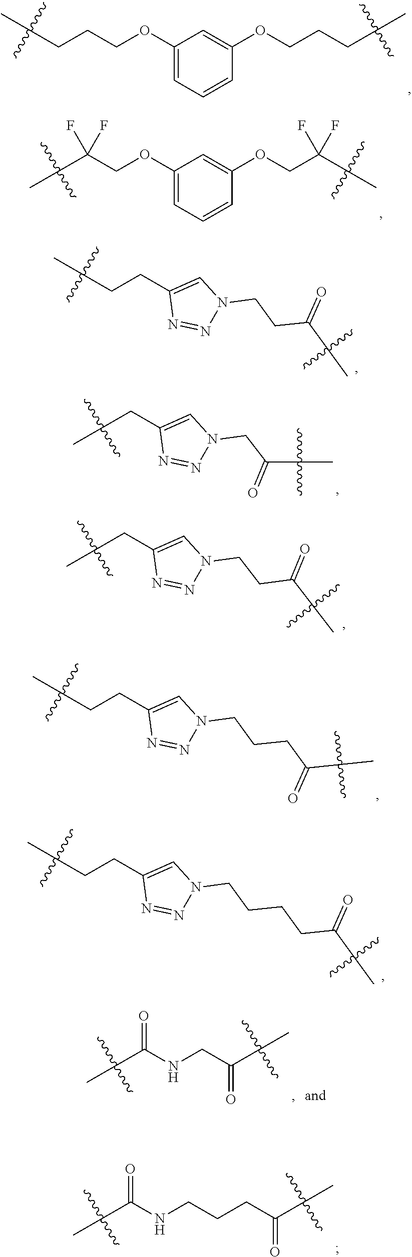

wherein “ [symbol] ” indicates a point of attachment.

In another embodiment of the present invention, there is provided a compound of Formula I or a pharmaceutically acceptable salt thereof for use in medical therapy.

In another embodiment of the present invention, there is provided a compound of Formula I or a pharmaceutically acceptable salt thereof for use in treating an HIV infection.

In another embodiment of the invention, there is provided a compound of Formula I wherein the compound or salt of the compound is used in the manufacture of a medicament for use in the treatment of an HIV infection in a human.

In some embodiments, the invention provides a method of curing an HIV infection in a subject comprising administering to the subject a compound of Formula I, as well as any compound of Table 1, along with pharmaceutically salts thereof. “Cure” or “Curing” a disease in a patient is used to denote the eradication, stoppage, halt or end of the human immunodeficiency virus or symptoms, or the progression of the symptoms or virus, for a defined period. As an example, in one embodiment, “cure” or “curing” refers to a therapeutic administration or a combination of administrations that alone or in combination with one or more other compounds induces and maintains sustained viral control (undetectable levels of plasma viremia by, e.g., a polymerase chain reaction (PCR) test, a bDNA (branched chain DNA) test or a NASBA (nucleic acid sequence based amplification) test,) of human immunodeficiency virus after a minimum of two years without any other therapeutic intervention. The above PCR, bDNA and NASBA tests are carried out using techniques known and familiar to one skilled in the art. As an example, the eradication, stoppage, halt or end of the human immunodeficiency virus or symptoms, or the progression of the symptoms or virus, may be sustained for a minimum of two years.

In some embodiments, the invention provides a method of curing an HIV infection in a subject comprising administering to the subject a pharmaceutical composition comprising a compound of Formula I, along with pharmaceutically salts thereof.

In some embodiments, the invention provides the use of a compound of Formula I, or a pharmaceutically acceptable salt thereof in the manufacture of a medicament for use in curing an HIV infection.

In some embodiments, the invention provides a compound of Formula I, or a pharmaceutically acceptable salt thereof for use in curing an HIV infection.

Combinations of compounds of Formulas I, and one or more agents useful in HIV therapy may also be used in methods of curing an HIV infection.

In one embodiment, the pharmaceutical formulation containing a compound of Formula I, or a salt thereof is a formulation adapted for parenteral administration. In another embodiment, the formulation is a long-acting parenteral formulation. In a further embodiment, the formulation is a nano-particle formulation.

The compounds of the present invention and their salts, solvates, or other pharmaceutically acceptable derivatives thereof, may be employed alone or in combination with other therapeutic agents. Therefore, in other embodiments, the methods of treating and/or preventing an HIV infection in a subject may in addition to administration of a compound of Formula I further comprising administration of one or more additional pharmaceutical agents active against HIV.

In such embodiments, the one or more additional agents active against HIV is selected from the group consisting of anti-retroviral agents, latency reversing agents, and agents for clearance therapy.

In other embodiments, the one or more additional agents active against HIV is selected from the group consisting of nucleotide reverse transcriptase inhibitors, non-nucleotide reverse transcriptase inhibitors, protease inhibitors, entry inhibitors, attachment and fusion inhibitors, integrase inhibitors, maturation inhibitors, CXCR4 and/or CCR5 inhibitors, histone deacetylase inhibitors, histone crotonyl transferase inhibitors, protein kinase C agonists, proteasome inhibitors, TLR7 agonists, bromodomain inhibitors, and neutralizing antibodies, and combinations thereof.

In certain embodiments, the one or more additional agents active against HIV is selected from the group consisting of zidovudine, didanosine, lamivudine, zalcitabine, abacavir, stavudine, adefovir, adefovir dipivoxil, fozivudine, todoxil, emtricitabine, alovudine, amdoxovir, elvucitabine, nevirapine, delavirdine, efavirenz, loviride, immunocal, oltipraz, capravirine, lersivirine, GSK2248761, TMC-278, TMC-125, etravirine, saquinavir, ritonavir, indinavir, nelfinavir, amprenavir, fosamprenavir, brecanavir, darunavir, atazanavir, tipranavir, palinavir, lasinavir, enfuvirtide, T-20, T-1249, PRO-542, PRO-140, TNX-355, BMS-806, BMS-663068 and BMS-626529, 5-Helix, raltegravir, elvitegravir, dolutegravir, cabotegravir, bictegravir, vicriviroc (Sch-C), Sch-D, TAK779, maraviroc, TAK449, didanosine, tenofovir, lopinavir, darunavir, vorinostat, panobinostat, romidepin, valpronic acid, mocetinostat, sodium corotonate, bryostatin, ingenol B, disulforam, GS-9620, JQ1, iBET151, bortezomib, epigallocatechin gallate, salinosporamide A, carfilzomib, broadly neutralizing antibodies (bNAb), eCD4-Ig, CD4-Ig, and dual-affinity re-targeting (DART) proteins.

As such, the compounds of the present invention of Formula (I) and any other pharmaceutically active agent(s) may be administered together or separately and, when administered separately, administration may occur simultaneously or sequentially, in any order. The amounts of the compounds of Formula (I) of the present invention and the other pharmaceutically active agent(s) and the relative timings of administration will be selected in order to achieve the desired combined therapeutic effect. The administration in combination of a compound of the present invention of Formula (I) and salts, solvates, or other pharmaceutically acceptable derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time. The amounts of the compound(s) of Formula (I) or salts thereof and the other pharmaceutically active agent(s) and the relative timings of administration will be selected in order to achieve the desired combined therapeutic effect.

In addition, the compounds of the present invention of Formula (I) may be used in combination with one or more other agents that may be useful in the treatment of HIV. They agents may include anti-retroviral agents, latency reversing agents, and agents for clearance therapy. Several examples of anti-retroviral agents are provided below:
Nucleotide reverse transcriptase inhibitors such as zidovudine, didanosine, lamivudine, zalcitabine, abacavir, stavudine, adefovir, adefovir dipivoxil, fozivudine, todoxil, emtricitabine, alovudine, amdoxovir, elvucitabine, and similar agents;
Non-nucleotide reverse transcriptase inhibitors (including an agent having anti-oxidation activity such as immunocal, oltipraz, etc.) such as nevirapine, delavirdine, efavirenz, loviride, immunocal, oltipraz, capravirine, lersivirine, GSK2248761, TMC-278, TMC-125, etravirine, and similar agents;
Protease inhibitors such as saquinavir, ritonavir, indinavir, nelfinavir, amprenavir, fosamprenavir, brecanavir, darunavir, atazanavir, tipranavir, palinavir, lasinavir, and similar agents;
Entry, attachment and fusion inhibitors such as enfuvirtide (T-20), T-1249, PRO-542, PRO-140, TNX-355, BMS-806, BMS-663068 and BMS-626529, 5-Helix and similar agents;
Integrase inhibitors such as raltegravir, elvitegravir, dolutegravir, cabotegravir, bictegravir and similar agents;
Maturation inhibitors such as PA-344 and PA-457, and similar agents; and
CXCR4 and/or CCR5 inhibitors such as vicriviroc (Sch-C), Sch-D, TAK779, maraviroc (UK 427,857), TAK449, as well as those disclosed in WO 02/74769, PCT/US03/39644, PCT/US03/39975, PCT/US03/39619, PCT/US03/39618, PCT/US03/39740, and PCT/US03/39732, and similar agents.

Further examples where the compounds of the present invention may be used in combination with one or more agents useful in the prevention or treatment of HIV are found in Table 2.

TABLE 2

| | Brand | | |
|---|---|---|---|
| FDA Approval | Name | Generic Name | Manufacturer |
| Nucleoside Reverse | | | |
| Transcriptase Inhibitors | | | |
| (NRTIs) | | | |
| 1987 | Retrovir | zidovudine, | GlaxoSmithKline |
| | | azidothymidine, | |
| | | AZT, ZDV | |
| 1991 | Videx | didanosine, | Bristol-Myers |
| | | dideoxyinosine, | Squibb |
| | | ddI | |
| 1992 | Hivid | zalcitabine, | Roche |
| | | dideoxycytidine, | Pharmaceuticals |
| | | ddC | |
| 1994 | Zerit | stavudine, d4T | Bristol-Myers |
| | | | Squibb |
| 1995 | Epivir | lamivudine, 3TC | GlaxoSmithKline |
| 1997 | Combivir | lamivudine + | GlaxoSmithKline |
| | | zidovudine | |
| 1998 | Ziagen | abacavir sulfate, | GlaxoSmithKline |
| | | ABC | |
| 2000 | Trizivir | abacavir + | GlaxoSmithKline |
| | | lamivudine + | |
| | | zidovudine | |
| 2000 | Videx EC | enteric coated | Bristol-Myers |
| | | didanosine, ddI EC | Squibb |
| 2001 | Viread | tenofovir | Gilead Sciences |
| | | disoproxil | |
| | | fumarate, TDF | |
| 2003 | Emtriva | emtricitabine, FTC | Gilead Sciences |
| 2004 | Epzicom | abacavir + | GlaxoSmithKline |
| | | lamivudine | |
| 2004 | Truvada | emtricitabine + | Gilead Sciences |
| | | tenofovir | |
| | | disoproxil | |
| | | fumarate | |
| Non-Nucleosides | | | |
| Reverse Transcriptase | | | |
| Inhibitors (NNRTIs) | | | |
| 1996 | Viramune | nevirapine, NVP | Boehringer |
| | | | Ingelheim |
| 1997 | Rescriptor | delavirdine, DLV | Pfizer |
| 1998 | Sustiva | efavirenz, EFV | Bristol-Myers |
| | | | Squibb |
| 2008 | Intelence | Etravirine | Tibotec |
| | | | Therapeutics |
| Protease | | | |
| Inhibitors (PIS) | | | |
| 1995 | Invirase | saquinavir | Roche |
| | | mesylate, SQV | Pharmaceuticals |
| 1996 | Norvir | ritonavir, RTV | Abbott |
| | | | Laboratories |
| 1996 | Crixivan | indinavir, IDV | Merck |
| 1997 | Viracept | nelfinavir | Pfizer |
| | | mesylate, NFV | |
| 1997 | Fortovase | saquinavir (no | Roche |
| | | longer marketed) | Pharmaceuticals |
| 1999 | Agenerase | amprenavir, APV | GlaxoSmithKline |
| 2000 | Kaletra | lopinavir + | Abbott |
| | | ritonavir, | Laboratories |
| | | LPV/RTV | |
| 2003 | Reyataz | atazanavir sulfate, | Bristol-Myers |
| | | ATV | Squibb |
| 2003 | Lexiva | fosamprenavir | GlaxoSmithKline |
| | | calcium, FOS- | |
| | | APV | |
| 2005 | Aptivus | tripranavir, TPV | Boehringer |
| | | | Ingelheim |
| 2006 | Prezista | Darunavir | Tibotec |
| | | | Therapeutics |
| Fusion Inhibitors | | | |
| 2003 | Fuzeon | Enfuvirtide, T-20 | Roche |
| | | | Pharmaceuticals & |
| | | | Trimeris |
| Entry Inhibitors | | | |
| 2007 | Selzentry | Maraviroc | Pfizer |
| Integrase Inhibitors | | | |
| 2007 | Isentress | Raltegravir | Merck |
| 2013 | Tivicay | Dolutegravir | ViiV Healthcare |
| — | — | Cabotegravir | |

The present invention may be used in combination with other agents that induce HIV expression, such as latency reversing agents. Several latency reversing agents include, but are not limited to, the following: histone deacetylase inhibitors (e.g., vorinostat, panobinostat, romidepin), histone crotonyl transferase inhibitors (sodium corotonate), protein kinase C agonists (e.g., bryostatin, ingenol B), disulfiram, TLR7 agonists (e.g., GS-9620), bromodomain inhibitors (e.g., JQ1, iBET151). Many of these agents are described in further detail below.

The present invention may be used in combination with other agents that induce HIV expression, such as agents for clearance therapy. Several examples of agents for clearance therapy, or of immunological combinations for clearance, include, but are not limited to, the following: neutralizing and broadly neutralizing antibodies (bNAb), eCD4-Ig, CD4-Ig, and dual-affinity re-targeting (DART) proteins.

The scope of combinations of compounds of this invention with HIV agents is not limited to those mentioned above, but includes in principle any combination with any pharmaceutical composition useful for the treatment and/or prevention of HIV. As noted, in such combinations the compounds of the present invention and other HIV agents may be administered separately or in conjunction. In addition, one agent may be prior to, concurrent to, or subsequent to the administration of other agent(s).

In some embodiments, the compounds of Formula I and Table 1 may be used in combination with one or more agents useful as pharmacological enhancers as well as with or without additional compounds for the prevention or treatment of HIV. Examples of such pharmacological enhancers (or pharmakinetic boosters) include, but are not limited to, ritonavir, GS-9350 (cobicistat), and SPI-452.

Ritonavir is 10-hydroxy-2-methyl-5-(1-methyethyl)-1-1[2-(1-methylethyl)-4-thiazolyl]-3,6-dioxo-8,11-bis(phenylmethyl)-2,4,7,12-tetraazatridecan-13-oic acid, 5-thiazolylmethyl ester, [5S-(5S*,8R*,10R*,11R*)] and is available from Abbott Laboratories of Abbott Park, Illinois, as Norvir. Ritonavir is an HIV protease inhibitor indicated with other antiretroviral agents for the treatment of HIV infection. Ritonavir also inhibits P450 mediated drug metabolism as well as the P-glycoprotein (Pgp) cell transport system, thereby resulting in increased concentrations of active compound within the organism.

GS-9350 (cobicistat) is a compound being developed by Gilead Sciences of Foster City California as a pharmacological enhancer.

SPI-452 is a compound being developed by Sequoia Pharmaceuticals of Gaithersburg, Maryland, as a pharmacological enhancer.

In some embodiments, a compound of Formula I is used in combination with ritonavir. In one embodiment, the combination is an oral fixed dose combination. In another embodiment, the compound of Formula I is formulated as a long-acting parenteral injection and ritonavir is formulated as an oral composition. In one embodiment, a kit containing the compound of Formula I is formulated as a long-acting parenteral injection and ritonavir formulated as an oral composition. In another embodiment, the compound of Formula I is formulated as a long-acting parenteral injection and ritonavir is formulated as an injectable composition. In one embodiment, a kit containing the compound of Formula I is formulated as a long-acting parenteral injection and ritonavir formulated as an injectable composition.

In some embodiments, a compound of Formula I is used in combination with GS-9350. In one embodiment, the combination is an oral fixed dose combination. In another embodiment, the compound of Formula I is formulated as a long-acting parenteral injection and GS-9350 is formulated as an oral composition. In one embodiment, there is provided a kit containing the compound of Formula I is formulated as a long-acting parenteral injection and GS-9350 formulated as an oral composition. In another embodiment, the compound of Formula I is formulated as a long-acting parenteral injection and GS-9350 is formulated as an injectable composition. In one embodiment, is a kit containing the compound of Formula I is formulated as a long-acting parenteral injection and GS-9350 formulated as an injectable composition.

In some embodiments, a compound of Formula I is used in combination with SPI-452. In one embodiment, the combination is an oral fixed dose combination. In other embodiments, the compound of Formula I is formulated as a long-acting parenteral injection and SPI-452 is formulated as an oral composition. In still other embodiments, there is provided a kit containing the compound of Formula I is formulated as a long-acting parenteral injection and SPI-452 formulated as an oral composition. In other embodiments, the compound of Formula I is formulated as a long-acting parenteral injection and SPI-452 is formulated as an injectable composition. In some embodiments, there is provided a kit containing the compound of Formula I is formulated as a long-acting parenteral injection and SPI-452 formulated as an injectable composition.

In some embodiments, a compound of Formula I is used in combination with compounds which are found in previously filed PCT/CN2011/0013021, which is herein incorporated by reference.

The above other therapeutic agents, when employed in combination with the chemical entities described herein, may be used, for example, in those amounts indicated in the Physicians' Desk Reference (PDR) or as otherwise determined by one of ordinary skill in the art.

In other embodiments, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula I.

In still other embodiments, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula I, wherein said virus is an HIV virus. In some embodiments, the HIV virus is the HIV-1 virus.

In other embodiments, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula I, further comprising administration of a therapeutically effective amount of one or more agents active against an HIV virus.

In some embodiments, there is provided a method for treating a viral infection in a mammal mediated at least in part by a virus in the retrovirus family of viruses which method comprises administering to a mammal, that has been diagnosed with said viral infection or is at risk of developing said viral infection, a compound of Formula I, further comprising administration of a therapeutically effective amount of one or more agents active against the HIV virus, wherein said agent active against HIV virus is selected from Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

In another aspect, a method of depleting latent HIV infected cells comprising administering to a subject a compound of Formula (I) or a pharmaceutically acceptable salt thereof.

In various embodiments, the compound is selected from the group consisting of those compounds listed in Table 1. In some embodiments, a pharmaceutical composition comprising this compound, or a pharmaceutically acceptable salt thereof, and a pharmaceutically acceptable excipient including e.g., those set forth herein. In other embodiments, a method of treating an HIV infection in a subject comprising administering to the subject this compound or a pharmaceutically acceptable salt thereof, as well as combinations. In still other embodiments, a compound of Formula I or Table 1, or a pharmaceutically acceptable salt thereof, for use in treating an HIV infection. Some embodiments also include use of this compound, in the manufacture of a medicament for treating an HIV infection. Still other embodiments include a method of depleting latent HIV infected cells comprising administering to a subject this compound or a pharmaceutically acceptable salt thereof, as well as combinations thereof.

In various embodiments, the method of depleting latent HIV infection further comprises administering to the subject one or more additional agents active against HIV as disclosed hereinabove. As an example, in various embodiments, the one or more additional agents is selected from the group consisting of nucleotide reverse transcriptase inhibitors, non-nucleotide reverse transcriptase inhibitors, protease inhibitors, entry inhibitors, attachment and fusion inhibitors, integrase inhibitors, maturation inhibitors, CXCR4 and/or CCR5 inhibitors, histone deacetylase inhibitors, histone crotonyl transferase inhibitors, protein kinase C agonists, proteasome inhibitors, TLR7 agonists, bromodomain inhibitors, and antibodies for clearance therapy, and combinations thereof. In various embodiments, the one or more additional agents active against HIV is selected from the group consisting of zidovudine, didanosine, lamivudine, zalcitabine, abacavir, stavudine, adefovir, adefovir dipivoxil, fozivudine, todoxil, emtricitabine, alovudine, amdoxovir, elvucitabine, nevirapine, delavirdinee, efavirenz, loviride, immunocal, oltipraz, capravirine, lersivirine, GSK2248761, TMC-278, TMC-125, etravirine, saquinavir, ritonavir, indinavir, nelfinavir, amprenavir, fosamprenavir, brecanavir, darunavir, atazanavir, tipranavir, palinavir, lasinavir, enfuvirtide, T-20, T-1249, PRO-542, PRO-140, TNX-355, BMS-806, BMS-663068 and BMS-626529, 5-Helix, raltegravir, elvitegravir, dolutegravir, cabotegravir, bictegravir, vicriviroc (Sch-C), Sch-D, TAK779, maraviroc, TAK449, didanosine, tenofovir, lopinavir, darunavir, vorinostat, panobinostat, romidepin, valpronic acid, mocetinostat, sodium corotonate, bryostatin, ingenol B, disulforam, GS-9620, JQ1, iBET151, bortezomib, epigallocatechin gallate, salinosporamide A, carfilzomib, and neutralizing antibodies, eCD4-Ig, CD4-Ig, bNAb, DARTS and IgA.

The compounds according to Formula I and pharmaceutically acceptable salts thereof may be useful in the treatment of cancer, pre-cancerous syndromes. Suitably the present invention relates to a method for treating cancers selected from the group consisting of brain (gliomas), glioblastomas, astrocytomas, glioblastoma multiforme, Bannayan-Zonana syndrome, Cowden disease, Lhermitte-Duclos disease, Wilm's tumor, Ewing's sarcoma, Rhabdomyosarcoma, ependymoma, medulloblastoma, head and neck, kidney, liver, melanoma, ovarian, pancreatic, adenocarcinoma, ductal adenocarcinoma, adenosquamous carcinoma, acinar cell carcinoma, glucagonoma, insulinoma, prostate, sarcoma, osteosarcoma, giant cell tumor of bone, thyroid, lymphoblastic T cell leukemia, chronic myelogenous leukemia, chronic lymphocytic leukemia, hairy-cell leukemia, acute lymphoblastic leukemia, acute myelogenous leukemia, chronic neutrophilic leukemia, acute lymphoblastic T cell leukemia, plasmacytoma, Immunoblastic large cell leukemia, mantle cell leukemia, multiple myeloma, megakaryoblastic leukemia, multiple myeloma, acute megakaryocytic leukemia, promyelocytic leukemia, erythroleukemia, malignant lymphoma, hodgkins lymphoma, non-hodgkins lymphoma, lymphoblastic T cell lymphoma, Burkitt's lymphoma, follicular lymphoma, neuroblastoma, bladder cancer, urothelial cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Suitably the present invention relates to a method for treating pre-cancerous syndromes in a mammal, including a human, wherein the pre-cancerous syndrome is selected from: cervical intraepithelial neoplasia, monoclonal gammapathy of unknown significance (MGUS), myelodysplastic syndrome, aplastic anemia, cervical lesions, skin nevi (pre-melanoma), prostatic intraepithleial (intraductal) neoplasia (PIN), Ductal Carcinoma in situ (DCIS), colon polyps and severe hepatitis or cirrhosis.

The compounds of Formula (I) and pharmaceutically acceptable salts thereof may be co-administered with at least one other active agent known to be useful in the treatment of cancer or pre-cancerous syndromes.

By the term “co-administration” as used herein is meant either simultaneous administration or any manner of separate sequential administration of a c-MYC inhibiting compound, as described herein, and a further active agent or agents, known to be useful in the treatment of cancer, including chemotherapy and radiation treatment. The term further active agent or agents, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered by injection and another compound may be administered orally.

Examples of a further active ingredient or ingredients (anti-neoplastic agent) for use in combination or co-administered with the presently invented combinations are indicated below. This list is non-limiting. Additional anti-neoplastic agents are contemplated for use with the presently invented compounds.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; cell cycle signaling inhibitors; proteasome inhibitors; and inhibitors of cancer metabolism.

Examples of a further active ingredient or ingredients (anti-neoplastic agent) for use in combination or co-administered with the presently invented compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)-N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p.16-23, 1995).

Docetaxel, (2R,3S)-N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4‘-deoxy-C’-norvincaleukoblastine [R-(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first- and second-line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting, and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids, leading to cell death. Examples of antibiotic anti-neoplastic agents include, but are not limited to, actinomycins such as dactinomycin, anthrocyclins such as daunorubicin and doxorubicin; and bleomycins.

Dactinomycin, also known as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S, 10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed, and cell death follows. Examples of antimetabolite anti-neoplastic agents include, but are not limited to, fluorouracil, methotrexate, cytarabine, mecaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythron-hydroxy-nonyl-adenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl) methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary, and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I—DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I—DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal transduction inhibitors useful in the present invention include inhibitors of receptor tyrosine kinases, non-receptor tyrosine kinases, SH2/SH3 domain blockers, serine/threonine kinases, phosphotidylinositol-3 kinases, myo-inositol signaling, and Ras oncogenes.

Several protein tyrosine kinases catalyze the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor—I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Suitably, the pharmaceutically active compounds of the invention are used in combination with a VEGFR inhibitor, suitably 5-[[4-[(2,3-dimethyl-2H-indazol-6-yl)methylamino]-2-pyrimidinyl]amino]-2-methylbenzenesulfonamide, or a pharmaceutically acceptable salt, suitably the monohydrochloride salt thereof, which is disclosed and claimed in in International Application No. PCT/US01/49367, having an International filing date of Dec. 19, 2001, International Publication Number WO02/059110 and an International Publication date of Aug. 1, 2002, the entire disclosure of which is hereby incorporated by reference, and which is the compound of Example 69. 5-[[4-[(2,3-dimethyl-2H-indazol-6-yl)methylamino]-2-pyrimidinyl]amino]-2-methylbenzenesulfonamide can be prepared as described in International Application No. PCT/US01/49367.

Suitably, 5-[[4-[(2,3-dimethyl-2H-indazol-6-yl)methylamino]-2-pyrimidinyl]amino]-2-methylbenzenesulfonamide is in the form of a monohydrochloride salt. This salt form can be prepared by one of skill in the art from the description in International Application No. PCT/US01/49367, having an International filing date of Dec. 19, 2001.

5-[[4-[(2,3-dimethyl-2H-indazol-6-yl)methylamino]-2-pyrimidinyl]amino]-2-methylbenzenesulfonamide is sold commercially as the monohydrochloride salt and is known by the generic name pazopanib and the trade name VOTRIENT©.

Pazopanib is implicated in the treatment of cancer and ocular diseases/angiogenesis. Suitably the present invention relates to the treatment of cancer and ocular diseases/angiogenesis, suitably age-related macular degeneration, which method comprises the administration of a compound of Formula (I) alone or in combination with pazopanib.

Tyrosine kinases, which are not growth factor receptor kinases are termed non-receptor tyrosine kinases. Non-receptor tyrosine kinases for use in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S. and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta). IkB kinase family (IKKa, IKKb), PKB family kinases, akt kinase family members, PDK1 and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; Pearce, L. R et al. Nature Reviews Molecular Cell Biology (2010) 11, 9-22. and Martinez-Iacaci, L., et al, Int. J. Cancer (2000), 88(1), 44-52.

Suitably, the pharmaceutically active compounds of the invention are used in combination with a MEK inhibitor. Suitably, N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate, suitably the dimethyl sulfoxide solvate, thereof, which is disclosed and claimed in International Application No. PCT/JP2005/011082, having an International filing date of Jun. 10, 2005; International Publication Number WO 2005/121142 and an International Publication date of Dec. 22, 2005, the entire disclosure of which is hereby incorporated by reference. N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide, can be prepared as described in United States Patent Publication No. US 2006/0014768, Published Jan. 19, 2006, the entire disclosure of which is hereby incorporated by reference.

Suitably, the pharmaceutically active compounds of the invention are used in combination with a B-Raf inhibitor. Suitably, N-{3-[5-(2-Amino-4-pyrimidinyl)-2-(1,1-dimethylethyl)-1,3-thiazol-4-yl]-2-fluorophenyl}-2,6-difluorobenzenesulfonamide, or a pharmaceutically acceptable salt thereof, which is disclosed and claimed, in International Application No. PCT/US2009/042682, having an International filing date of May 4, 2009, the entire disclosure of which is hereby incorporated by reference. N-{3-[5-(2-Amino-4-pyrimidinyl)-2-(1,1-dimethylethyl)-1,3-thiazol-4-yl]-2-fluorophenyl}-2,6-difluorobenzenesulfonamide can be prepared as described in International Application No. PCT/US2009/042682.

Suitably, the pharmaceutically active compounds of the invention are used in combination with an Akt inhibitor. Suitably, N-{(1S)-2-amino-1-[(3,4-difluorophenyl)methyl]ethyl}-5-chloro-4-(4-chloro-1-methyl-1H-pyrazol-5-yl)-2-furancarboxamide or a pharmaceutically acceptable salt thereof, which is disclosed and claimed in International Application No. PCT/US2008/053269, having an International filing date of Feb. 7, 2008; International Publication Number WO 2008/098104 and an International Publication date of Aug. 14, 2008, the entire disclosure of which is hereby incorporated by reference. N-{(1S)-2-amino-1-[(3,4-difluorophenyl)methyl]ethyl}-5-chloro-4-(4-chloro-1-methyl-1H-pyrazol-5-yl)-2-furancarboxamide is the compound of example 224 and can be prepared as described in International Application No. PCT/US2008/053269.

Suitably, the pharmaceutically active compounds of the invention are used in combination with an Akt inhibitor. Suitably, N-{(1S)-2-amino-1-[(3-fluorophenyl)methyl]ethyl}-5-chloro-4-(4-chloro-1-methyl-1H-pyrazol-5-yl)-2-thiophenecarboxamide or a pharmaceutically acceptable salt thereof, which is disclosed and claimed in International Application No. PCT/US2008/053269, having an International filing date of Feb. 7, 2008; International Publication Number WO 2008/098104 and an International Publication date of Aug. 14, 2008, the entire disclosure of which is hereby incorporated by reference. N-{(1S)-2-amino-1-[(3-fluorophenyl)methyl]ethyl}-5-chloro-4-(4-chloro-1-methyl-1H-pyrazol-5-yl)-2-thiophenecarboxamide is the compound of example 96 and can be prepared as described in International Application No. PCT/US2008/053269. Suitably, N-{(1S)-2-amino-1-[(3-fluorophenyl)methyl]ethyl}-5-chloro-4-(4-chloro-1-methyl-1H-pyrazol-5-yl)-2-thiophenecarboxamide is in the form of a hydrochloride salt. The salt form can be prepared by one of skill in the art from the description in International Application No. PCT/US2010/022323, having an International filing date of Jan. 28, 2010.

Inhibitors of Phosphotidylinositol-3 Kinase family members including blockers of PI3-kinase, ATM, DNA-PK, and Ku may also be useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also, of interest in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example, Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signaling in Breast cancer:erbB Family Receptor Tyrosine Kinases, Breast cancer Res., 2000, 2(3), 176-183); and 2CB VEGFR2 specific antibody (see Brekken, R. A. et al, Selective Inhibition of VEGFR2 Activity by a monoclonal Anti-VEGF antibody blocks tumor growth in mice, Cancer Res. (2000) 60, 5117-5124).

Non-receptor kinase angiogenesis inhibitors may also be useful in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Accordingly, non-receptor tyrosine kinase inhibitors may be used in combination with the compounds of the present invention. For example, anti-VEGF antibodies, which do not recognize VEGFR (the receptor tyrosine kinase), but bind to the ligand; small molecule inhibitors of integrin (alphaᵥ beta₃) that will inhibit angiogenesis; endostatin and angiostatin (non-RTK) may also prove useful in combination with the disclosed compounds. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of Formula (I). There are a number of immunologic strategies to generate an immune response. These strategies are generally in the realm of tumor vaccinations. The efficacy of immunologic approaches may be greatly enhanced through combined inhibition of signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signaling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signaling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230. Further, p21WAF1/CIP1 has been described as a potent and universal inhibitor of cyclin-dependent kinases (Cdks) (Ball et al., Progress in Cell Cycle Res., 3: 125 (1997)). Compounds that are known to induce expression of p21WAF1/CIP1 have been implicated in the suppression of cell proliferation and as having tumor suppressing activity (Richon et al., Proc. Nat Acad. Sci. U.S.A. 97(18): 10014-10019 (2000)), and are included as cell cycle signaling inhibitors. Histone deacetylase (HDAC) inhibitors are implicated in the transcriptional activation of p21WAF1/CIP1 (Vigushin et al., Anticancer Drugs, 13(1): 1-13 (January 2002)), and are suitable cell cycle signaling inhibitors for use in combination herein.

Examples of such HDAC inhibitors include:

1. Vorinostat, including pharmaceutically acceptable salts thereof. Marks et al., Nature Biotechnology 25, 84 to 90 (2007); Stenger, Community Oncology 4, 384-386 (2007).

Vorinostat has the following chemical structure and name:

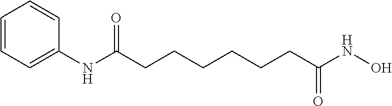 [SMILES: O=C(CCCCCCC(=O)Nc1ccccc1)NO]

N-hydroxy-N-phenyl-octanediamide

2. Romidepsin, including pharmaceutically acceptable salts thereof. Vinodhkumar et al., Biomedicine & Pharmacotherapy 62 (2008) 85-93. Romidepsin, has the following chemical structure and name:

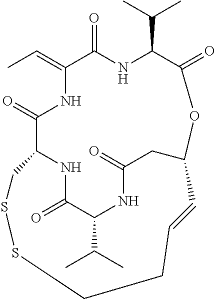 [SMILES: C/C=C1\NC(=O)[C@H]2CSSCC/C=C/[C@H](CC(=O)N[C@H](C(C)C)C(=O)N2)OC(=O)[C@H](C(C)C)NC1=O]

(1S,4S,7Z,10S,16E,21R)-7-ethylidene-4,21-di(propan-2-yl)-2-oxa-12,13-dithia-5,8,20,23-tetrazabicyclo[8.7.6]tricos-16-ene-3,6,9,19,22-pentone

3. Panobinostat, including pharmaceutically acceptable salts thereof. Drugs of the Future 32(4): 315-322 (2007).

Panobinostat, has the following chemical structure and name:

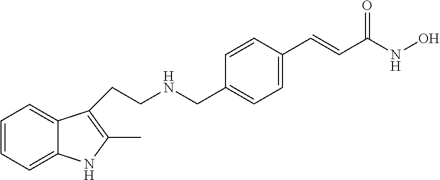 [SMILES: Cc1[nH]c2ccccc2c1CCNCc1ccc(/C=C/C(=O)NO)cc1]

(2E)-N-hydroxy-3-[4-({[2-(2-methyl-1H-indol-3-yl)ethyl]amino}methyl)phenyl]acrylamide

4. Valproic acid, including pharmaceutically acceptable salts thereof. Gottlicher, et al., EMBO J. 20(24): 6969-6978 (2001).

Valproic acid, has the following chemical structure and name:

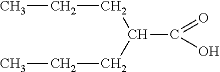

2-propylpentanoic acid

5. Mocetinostat (MGCD0103), including pharmaceutically acceptable salts thereof. Balasubramanian et al., Cancer Letters 280: 211-221 (2009). Mocetinostat, has the following chemical structure and name:

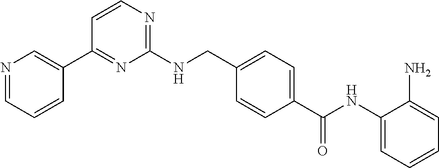 [SMILES: Nc1ccccc1NC(=O)c1ccc(CNc2nccc(-c3cccnc3)n2)cc1]

N-(2-Aminophenyl)-4-[[(4-pyridin-3-ylpyrimidin-2-yl)amino]methyl]benzamide

Further examples of such HDAC inhibitors are included in Bertrand European Journal of Medicinal Chemistry 45, (2010) 2095-2116, particularly the compounds of Table 3 therein as indicated below.

TABLE 3
Hydroxamic acids

| | |
|---|---|
| 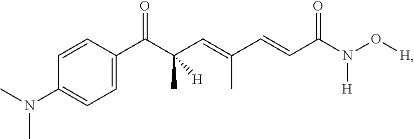 | 1 |
| Trichostatine A (TSA) | |
| 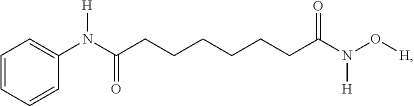 | 2 |
| SAHA | |
| 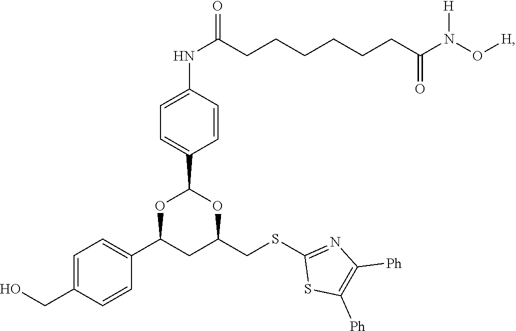 | 3 |
| Tubacin | |
| 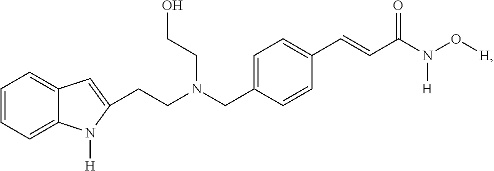 | 4 |
| LAQ824 | |
| 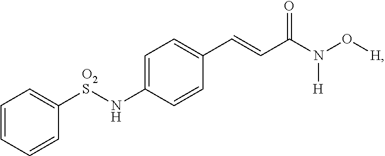 | 5 |
| Sulfonamide | |
| 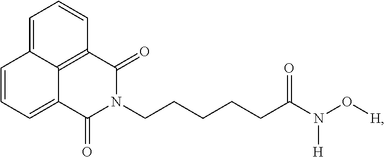 | 6 |
| Scriptaid | |
| 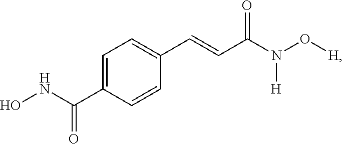 | 7 |
| CBHA | |
| 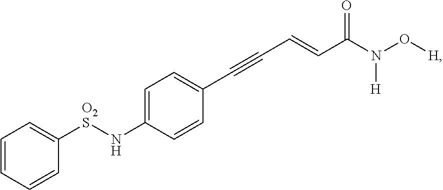 | 8 |
| Oxamflatin | |

Cyclic tetrapeptides

| | |
|---|---|
| 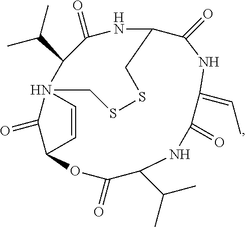 | 9 |
| FK228 | |
| 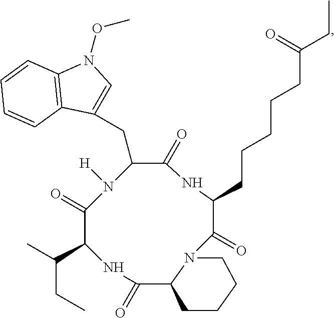 | 10 |
| Apicidin | |

Short chain carboxylic acids

| | |
|---|---|
| 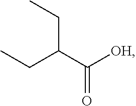 | 11 |
| Valproic acid | |
| 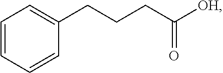 [SMILES: O=C(O)CCCc1ccccc1] | 12 |
| Phenylbutyric acid | |

Benzamides

| | |
|---|---|
| 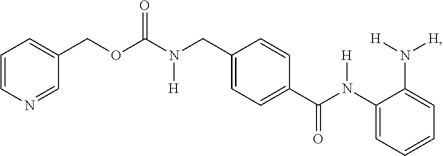 | 13 |
| MS-275 | |
| 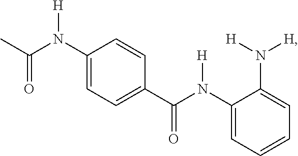 | 14 |
| Cl-994 | |

Keto derivatives

| | |
|---|---|
| 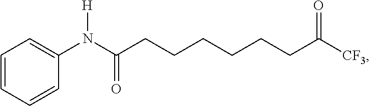 [SMILES: O=C(CCCCCCC(=O)C(F)(F)F)Nc1ccccc1] | 15 |
| Trifluoromethyl cetone | |
| 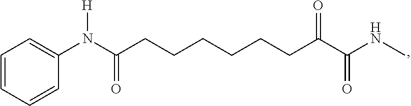 [SMILES: CNC(=O)C(=O)CCCCCCC(=O)Nc1ccccc1] | 16 |
| alpha-cetoamide | |

Proteasome inhibitors are drugs that block the action of proteasomes, cellular complexes that break down proteins, like the p53 protein. Several proteasome inhibitors are marketed or are being studied in the treatment of cancer. Suitable proteasome inhibitors for use in combination herein include:

1. Bortezomib (Velcade®), including pharmaceutically acceptable salts thereof. Adams J, Kauffman M (2004), Cancer Invest 22 (2): 304-11. Bortezomib has the following chemical structure and name.

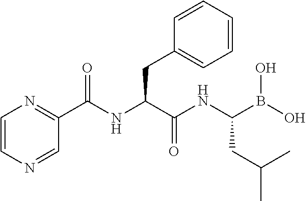 [SMILES: CC(C)C[C@H](NC(=O)[C@H](Cc1ccccc1)NC(=O)c1cnccn1)B(O)O]

[(1R)-3-methyl-1-({(2S)-3-phenyl-2-[(pyrazin-2-ylcarbonyl)amino]propanoyl}amino)butyl]boronic acid

2. Disulfiram, including pharmaceutically acceptable salts thereof. Bouma et al. (1998). J. Antimicrob. Chemother. 42 (6): 817-20. Disulfiram has the following chemical structure and name.

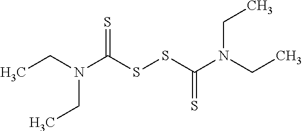 [SMILES: CCN(CC)C(=S)SSC(=S)N(CC)CC]

1,1′,1″,1′″-[disulfanediylbis(carbonothioylnitrilo)]tetraethane

3. Epigallocatechin gallate (EGCG), including pharmaceutically acceptable salts thereof. Williamson et al., (December 2006), The Journal of Allergy and Clinical Immunology 118 (6): 1369-74.

Epigallocatechin gallate has the following chemical structure and name.

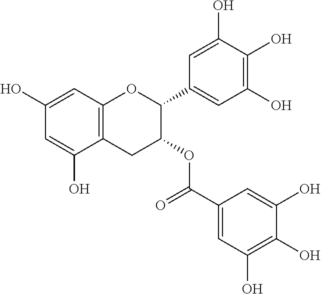 [SMILES: O=C(O[C@@H]1Cc2c(O)cc(O)cc2O[C@@H]1c1cc(O)c(O)c(O)c1)c1cc(O)c(O)c(O)c1]

[(2R,3R)-5,7-dihydroxy-2-(3,4,5-trihydroxyphenyl)chroman-3-yl]3,4,5-trihydroxybenzoate

4. Salinosporamide A, including pharmaceutically acceptable salts thereof. Feling et at., (2003), Angew. Chem. Int. Ed. Engl. 42 (3): 355-7.

Salinosporamide A has the following chemical structure and name.

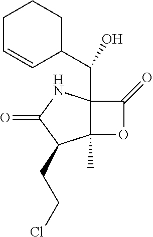 [SMILES: C[C@@]12OC(=O)C1([C@@H](O)C1C=CCCC1)NC(=O)[C@@H]2CCCl]

(4R,5S)-4-(2-chloroethyl)-1-((1S)-cyclohex-2-enyl(hydroxy)methyl)-5-methyl-6-oxa-2-azabicyclo3.2.0heptane-3,7-dione

5. Carfilzomib, including pharmaceutically acceptable salts thereof. Kuhn D J, et al, Blood, 2007, 110:3281-3290.

Carfilzomib has the following chemical structure and name.

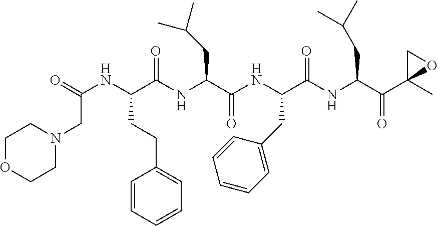 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

(S)-4-methyl-N-((S)-1-(((S)-4-methyl-1-((R)-2-methyloxiran-2-yl)-1-oxopentan-2-yl)amino)-1-oxo-3-phenylpropan-2-yl)-2-((S)-2-(2-morpholinoacetamido)-4-phenylbutanamido)pentanamide

The 70 kilodalton heat shock proteins (Hsp70s) and 90 kilodalton heat shock proteins (Hsp90s) are a families of ubiquitously expressed heat shock proteins. Hsp70s and Hsp90s are over expressed certain cancer types. Several Hsp70s and Hsp90s inhibitors are being studied in the treatment of cancer. Suitable Hsp70s and Hsp90s inhibitors for use in combination herein include:

1. 17-AAG (Geldanamycin), including pharmaceutically acceptable salts thereof. Jia W et al. Blood. 2003 Sep. 1; 102(5):1824-32.

17-AAG(Geldanamycin) has the following chemical structure and name.

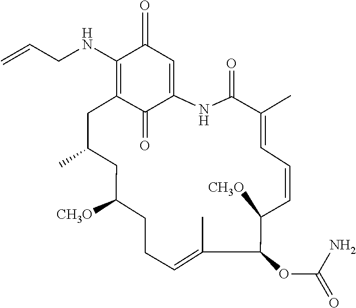

17-(Allylamino)-17-demethoxygeldanamycin

2. Radicicol, including pharmaceutically acceptable salts thereof (Lee et al., Mol Cell Endocrinol. 2002, 188, 47-54). Radicicol has the following chemical structure and name.

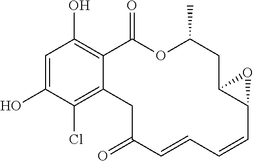 [SMILES: C[C@@H]1C[C@H]2O[C@@H]2/C=C\C=C\C(=O)Cc2c(Cl)c(O)cc(O)c2C(=O)O1]

(1aR,2Z,4E,14R,15aR)-8-chloro-9,11-dihydroxy-14-methyl-15,15a-dihydro-1aH-benzo[c]oxireno[2,3-k][1]oxacyclotetradecine-6,12(7H,14H)-dione

Inhibitors of cancer metabolism—Many tumor cells show a markedly different metabolism from that of normal tissues. For example, the rate of glycolysis, the metabolic process that converts glucose to pyruvate, is increased, and the pyruvate generated is reduced to lactate, rather than being further oxidized in the mitochondria via the tricarboxylic acid (TCA) cycle. This effect is often seen even under aerobic conditions and is known as the Warburg Effect.

Lactate dehydrogenase A (LDH-A), an isoform of lactate dehydrogenase expressed in muscle cells, plays a pivotal role in tumor cell metabolism by performing the reduction of pyruvate to lactate, which can then be exported out of the cell. The enzyme has been shown to be upregulated in many tumor types. The alteration of glucose metabolism described in the Warburg effect is critical for growth and proliferation of cancer cells and knocking down LDH-A using RNA-i has been shown to lead to a reduction in cell proliferation and tumor growth in xenograft models.
D. A. Tennant et. al., Nature Reviews, 2010, 267.
P. Leder, et. al., Cancer Cell, 2006, 9, 425.

High levels of fatty acid synthase (FAS) have been found in cancer precursor lesions. Pharmacological inhibition of FAS affects the expression of key oncogenes involved in both cancer development and maintenance.
Alli et al. Oncogene (2005) 24, 39-46. doi:10.1038

Inhibitors of cancer metabolism, including inhibitors of LDH-A and inhibitors of fatty acid biosynthesis (or FAS inhibitors), are suitable for use in combination with the compounds of this invention.

In one embodiment, the cancer treatment method of the claimed invention includes the co-administration a compound of Formula I and/or a pharmaceutically acceptable salt thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, cell cycle signaling inhibitors; proteasome inhibitors; and inhibitors of cancer metabolism.

In one embodiment, a compound of Formula I is used as a chemosensitizer to enhance tumor cell killing.

In one embodiment, a compound of Formula I is used in combination as a chemosensitizer to enhance tumor cell killing.

In one embodiment, a compound of Formula I is used in combination with a compound that inhibits the activity of protein kinase R (PKR)-like ER kinase, PERK (PERK inhibitor).

Suitably, the compounds of Formula I and pharmaceutically acceptable salts thereof may be co-administered with at least one other active agent known to be inhibitors of PERK kinase (EIF2K3) for treating or lessening the severity of neurodegenerative diseases/injury, such as Alzheimer's disease, spinal cord injury, traumatic brain injury, ischemic stroke, stroke, diabetes, Parkinson disease, Huntington's disease, Creutzfeldt-Jakob Disease, and related prion diseases, progressive supranuclear palsy, amyotrophic lateral sclerosis, myocardial infarction, cardiovascular disease, inflammation, fibrosis, chronic and acute diseases of the liver, chronic and acute diseases of the lung, chronic and acute diseases of the kidney, chronic traumatic encephalopathy (CTE), neurodegeneration, dementia, traumatic brain injury, cognitive impairment, atherosclerosis, ocular diseases, arrhythmias, in organ transplantation and in the transportation of organs for transplantation.

“Chemotherapeutic” or “chemotherapeutic agent” is used in accordance with its plain ordinary meaning and refers to a chemical composition or compound having antineoplastic properties or the ability to inhibit the growth or proliferation of cells.

Additionally, the compounds described herein can be co-administered with conventional immunotherapeutic agents including, but not limited to, immunostimulants (e.g., Bacillus Calmette-Guerin (BCG), levamisole, interleukin-2, alpha-interferon, etc.), monoclonal antibodies (e.g., anti-CD20, anti-HER2, anti-CD52, anti-HLA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), and radioimmunotherapy (e.g., anti-CD20 monoclonal antibody conjugated to ¹¹¹In, ⁹⁰Y, or ¹³¹I, etc.).

In a further embodiment, the compounds described herein can be co-administered with conventional radiotherapeutic agents including, but not limited to, radionuclides such as ⁴⁷Sc, ⁶⁴C ⁶⁷C, ⁸⁹Sr, ⁸⁶Y, ⁸⁷Y, and ²¹²Bi, optionally conjugated to antibodies directed against tumor antigens.

Additional examples of a further active ingredient or ingredients (anti-neoplastic agent) for use in combination or co-administered with the compounds are anti-PD-L1 agents.

Anti-PD-L1 antibodies and methods of making the same are known in the art.

Such antibodies to PD-L1 may be polyclonal or monoclonal, and/or recombinant, and/or humanized.

Exemplary PD-L1 antibodies are disclosed in:
U.S. Pat. No. 8,217,149; Ser. No. 12/633,339;
U.S. Pat. No. 8,383,796; Ser. No. 13/091,936;
U.S. Pat. No. 8,552,154; Ser. No. 13/120,406;
US patent publication No. 20110280877; Ser. No. 13/068,337;
US Patent Publication No. 20130309250; Ser. No. 13/892,671;
WO2013019906;
WO2013079174;
U.S. application Ser. No. 13/511,538 (filed Aug. 7, 2012), which is the US National Phase of International Application No. PCT/US10/58007 (filed 2010);
and
U.S. application Ser. No. 13/478,511 (filed May 23, 2012).

Additional exemplary antibodies to PD-L1 (also referred to as CD274 or B7-H1) and methods for use are disclosed in U.S. Pat. No. 7,943,743; US20130034559, WO2014055897, U.S. Pat. Nos. 8,168,179; 7,595,048. PD-L1 antibodies are in development as immuno-modulatory agents for the treatment of cancer.

In one embodiment, the antibody to PD-L1 is an antibody disclosed in U.S. Pat. No. 8,217,149. In another embodiment, the anti-PD-L1 antibody comprises the CDRs of an antibody disclosed in U.S. Pat. No. 8,217,149.

In another embodiment, the antibody to PD-L1 is an antibody disclosed in U.S. application Ser. No. 13/511,538. In another embodiment, the anti-PD-L1 antibody comprises the CDRs of an antibody disclosed in U.S. application Ser. No. 13/511,538.

In another embodiment, the antibody to PD-L1 is an antibody disclosed in application Ser. No. 13/478,511. In another embodiment, the anti-PD-L1 antibody comprises the CDRs of an antibody disclosed in U.S. application Ser. No. 13/478,511.

In one embodiment, the anti-PD-L1 antibody is BMS-936559 (MDX-1105). In another embodiment, the anti-PD-L1 antibody is MPDL3280A (RG7446). In another embodiment, the anti-PD-L1 antibody is MEDI4736.

Additional examples of a further active ingredient or ingredients (anti-neoplastic agent) for use in combination or co-administered with the presently invented ATF4 pathway inhibiting compounds are PD-1 antagonist.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCDIL2, PDL2, B7-DC, Btdc and CD273 for PD-L2. In any embodiments of the aspects or embodiments of the present invention in which a human individual is to be treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in the any of the aspects of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)2, scFv and Fv fragments.

Examples of mAbs that bind to human PD-1, and useful in the various aspects and embodiments of the present invention, are described in U.S. Pat. Nos. 7,488,802, 7,521,051, 8,008,449, 8,354,509, 8,168,757, WO2004/004771, WO2004/072286, WO2004/056875, and US2011/0271358.

Specific anti-human PD-1 mAbs useful as the PD-1 antagonist in any of the aspects and embodiments of the present invention include: MK-3475, a humanized IgG4 mAb with the structure described in WHO Drug Information, Vol. 27, No. 2, pages 161-162 (2013) and which comprises the heavy and light chain amino acid sequences shown in FIG. 6; nivolumab, a human IgG4 mAb with the structure described in WHO Drug Information, Vol. 27, No. 1, pages 68-69 (2013) and which comprises the heavy and light chain amino acid sequences shown in FIG. 7; the humanized antibodies h409A11, h409A16 and h409A17, which are described in WO2008/156712, and AMP-514, which is being developed by Medimmune.

Other PD-1 antagonists useful in the any of the aspects and embodiments of the present invention include an immunoadhesion that specifically binds to PD-1, and preferably specifically binds to human PD-1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, medicaments and uses of the present invention include AMP-224 (also known as B7-DCIg), which is a PD-L2-FC fusion protein and binds to human PD-1.

Other examples of mAbs that bind to human PD-L1, and useful in the treatment method, medicaments and uses of the present invention, are described in WO2013/019906, WO2010/077634 A1 and U.S. Pat. No. 8,383,796. Specific anti-human PD-L1 mAbs useful as the PD-1 antagonist in the treatment method, medicaments and uses of the present invention include MPDL3280A, BMS-936559, MEDI4736, MSB0010718C.

KEYTRUDA/pembrolizumab is an anti-PD-1 antibody marketed for the treatment of lung cancer by Merck. The amino acid sequence of pembrolizumab and methods of using are disclosed in U.S. Pat. No. 8,168,757.

Opdivo/nivolumab is a fully human monoclonal antibody marketed by Bristol Myers Squibb directed against the negative immunoregulatory human cell surface receptor PD-1 (programmed death-1 or programmed cell death-1/PCD-1) with immunopotentiation activity. Nivolumab binds to and blocks the activation of PD-1, an Ig superfamily transmembrane protein, by its ligands PD-L1 and PD-L2, resulting in the activation of T-cells and cell-mediated immune responses against tumor cells or pathogens. Activated PD-1 negatively regulates T-cell activation and effector function through the suppression of PI3k/Akt pathway activation. Other names for nivolumab include: BMS-936558, MDX-1106, and ONO-4538. The amino acid sequence for nivolumab and methods of using and making are disclosed in U.S. Pat. No. 8,008,449.

Additional examples of a further active ingredient or ingredients (anti-neoplastic agent) for use in combination or co-administered with the compounds of the invention are immuno-modulators.

As used herein “immuno-modulators” refer to any substance including monoclonal antibodies that affects the immune system. The ICOS binding proteins of the present invention can be considered immune-modulators. Immuno-modulators can be used as anti-neoplastic agents for the treatment of cancer. For example, immune-modulators include, but are not limited to, anti-CTLA-4 antibodies such as ipilimumab (YERVOY) and anti-PD-1 antibodies (Opdivo/nivolumab and Keytruda/pembrolizumab). Other immuno-modulators include, but are not limited to, OX-40 antibodies, PD-L1 antibodies, LAG3 antibodies, TIM-3 antibodies, 41BB antibodies and GITR antibodies.

Yervoy (ipilimumab) is a fully human CTLA-4 antibody marketed by Bristol Myers Squibb. The protein structure of ipilimumab and methods are using are described in U.S. Pat. Nos. 6,984,720 and 7,605,238.

In another embodiment, this invention provides a compound of Table 1 described herein or a Formula (I) or a pharmaceutically acceptable salt or prodrug thereof, for use in the treatment of a hepatitis B virus-related disease, condition or disorder. This invention provides a compound of Table 1 or a pharmaceutically acceptable salt or prodrug thereof, for use in the treatment of a hepatitis B virus-related disease, condition or disorder. wherein the hepatitis B virus-related disease, condition or disorder may be jaundice, liver cancer, liver inflammation, liver fibrosis, liver cirrhosis, liver failure, diffuse hepatocellular inflammatory disease, hemophagocytic syndrome or serum hepatitis).

In some embodiments, the compound of the present invention of Formula (I) or pharmaceutically acceptable salts thereof, is selected from the group of compounds set forth in Table 1. Additionally, the present invention also encompasses each of these compounds individually and pharmaceutically acceptable salts thereof.

In other embodiments, there is provided a pharmaceutical composition comprising a pharmaceutically acceptable diluent and a therapeutically effective amount of a compound of Formula I or a pharmaceutically acceptable salt thereof.

In some embodiments, the compound(s) of the present invention, or a pharmaceutically acceptable salt thereof, is chosen from the compounds set forth in Table 1. The compounds of the present invention can be supplied in the form of a pharmaceutically acceptable salt. The terms “pharmaceutically acceptable salt” refer to salts prepared from pharmaceutically acceptable inorganic and organic acids and bases. Accordingly, the word “or” in the context of “a compound or a pharmaceutically acceptable salt thereof” is understood to refer to either a compound or a pharmaceutically acceptable salt thereof (alternative), or a compound and a pharmaceutically acceptable salt thereof (in combination).

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication. The skilled artisan will appreciate that pharmaceutically acceptable salts of compounds according to Formula I may be prepared. These pharmaceutically acceptable salts may be prepared in situ during the final isolation and purification of the compound, or by separately reacting the purified compound in its free acid or free base form with a suitable base or acid, respectively.

Compounds of the invention may be made according to various schemes described below

### Synthetic Methods

The methods of synthesis for the provided chemical entities employ readily available starting materials using the following general methods and procedures. It will be appreciated that where typical or preferred process conditions (i.e., reaction temperatures, times, mole ratios of reactants, solvents, pressures, etc.) are given; other process conditions can also be used unless otherwise stated. Optimum reaction conditions may vary with the particular reactants or solvent used, but such conditions can be determined by one skilled in the art by routine optimization procedures.

Additionally, the methods of this invention may employ protecting groups which prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the provided chemical entities may contain one or more chiral centers and such compounds can be prepared or isolated as pure stereoisomers, i.e., as individual enantiomers or diastereomers, or as stereoisomer-enriched mixtures. All such stereoisomers (and enriched mixtures) are included within the scope of this specification, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Ernka-Chemce or Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4th Edition), and Larock's Comprehensive Organic Transformations (VCH Publishers Inc., 1989).

Unless specified to the contrary, the reactions described herein take place at atmospheric pressure, generally within a temperature range from −78 C to 200 C. Further, except as employed in the Examples or as otherwise specified, reaction times and conditions are intended to be approximate, e.g., taking place at about atmospheric pressure within a temperature range of about −78 C to about 110 C over a period of about 1 to about 24 hours; reactions left to run overnight average a period of about 16 hours.

The terms “solvent,” “organic solvent,” and “inert solvent” each mean a solvent inert under the conditions of the reaction being described in conjunction therewith, including, for example, benzene, toluene, acetonitrile, tetrahydrofuranyl (“THF”), dimethylformamide (“DMF”), chloroform, methylene chloride (or dichloromethane), diethyl ether, methanol, N-methylpyrrolidone (“NMP”), pyridine and the like.

Isolation and purification of the chemical entities and intermediates described herein

can be affected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which may be separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

### Examples

The following examples serve to more fully describe the manner of making and using the above-described invention. It is understood that these examples in no way serve to limit the true scope of the invention, but rather are presented for illustrative purposes. In the examples below and the synthetic schemes above, the following abbreviations have the following meanings. If an abbreviation is not defined, it has its generally accepted meaning.

| | | |
|---|---|---|
| ACN, AcCN, | = | acetonitrile |
| CH₃CN, or MeCN | | |
| AcOH | = | acetic acid |
| aq. | = | aqueous |
| Boc₂O | = | di-tert-butyl dicarbonate |
| Boc | = | tert-butoxycarbonyl |
| BPO | = | benzoyl peroxide |
| br | = | broad |
| d | = | doublet |
| ° C. | = | degrees Celsius |
| DCM | = | dichloromethane |
| dd | = | doublet of doublets |
| DIAD | = | diisopropyl azodicarboxylate |
| DIEA | = | diisopropylethylamine |
| DMAP | = | 4-(dimethylamino)pyridine |
| DMF | = | N,N-dimethylformamide |
| DMSO | = | dimethylsulfoxide |
| EDC or EDCI | = | 1-ethyl-3-(3-dimethylaminopropyl) |
| | | carbodiimide |
| EtOAc | = | ethyl acetate |
| FA | = | formic acid |
| g | = | gram |
| h or hr | = | hours |
| H₂O | = | water |
| HATU | = | Hexafluorophosphate Azabenzotriazole |
| | | Tetramethyl Uronium |
| HBr | = | hydrogen bromide |
| HPLC | = | high performance liquid chromatography |
| Hz | = | Hertz |
| IC₅₀ | = | inhibitory concentration at 50% inhibition |
| J | = | coupling constant (given in Hz unless otherwise |
| | | indicated) |
| KOAc | = | potassium acetate |
| LCMS or LC-MS | | liquid chromatography mass spectroscopy |
| LHMDS | = | lithium bis(trimethylsilyl)amide |
| m | = | multiplet |
| M | = | molar |
| M+H⁺ | = | parent mass spectrum peak plus H⁺ |
| μL | = | Microliters |
| μM | = | Micromolar |
| m-CPBA | = | meta-chloroperoxybenzoic acid |
| MeOH or CH₃OH | = | methanol |
| mg | = | milligram |
| min | = | minutes |
| mL | = | milliliter |
| mM | = | millimolar |
| mmol | = | millimole |
| MPLC | = | medium pressure liquid chromatography |
| MS | = | mass spectrum |
| MsCl | = | methanesulfonyl chloride |
| N₂ | = | nitrogen |
| NaH | = | sodium hydride |
| NaHCO₃ | = | Sodium bicarbonate |
| Na₂SO₄ | = | sodium sulfate |
| NBS | = | N-bromosuccinimide |
| nm | = | nanomolar |
| NMR | = | nuclear magnetic resonance |
| PPh₃ or TPP | = | triphenylphosphine |
| ppm | = | parts per million |
| psi | = | pounds per square inch |
| RT | = | room temperature |
| s | = | singlet |
| sat. or satd. | = | saturated |
| t | = | triplet |
| TBS-Cl or TBDMS- | = | tert-Butyldimethylsilyl chloride |
| Cl | | |
| TEA | = | triethylamine |
| THF | = | tetrahydrofuran |
| TFA | = | trifluoroacetic acid |
| TLC | = | thin layer chromatography |

### Equipment Description

NMR Instrumentation and data:

¹H NMR spectra were recorded at 25° C. on various instruments as listed below with all spectrometers operating at 400 MHz.
Bruker AVANCE NEO 400 MHz/54 mm instrument (MRCA 400/54/ASC, 16971)
Bruker AVANCE III 400 MHz/54 mm UltraShield Plus, long hold time, instrument (BZH 439′400′70I, D335/54-6776)
Bruker AVANCE III 400 MHz/54 mm Ascend instrument (BZH 994′400′70I, D315′54-9223)
Varian 400MR 400 MHz/54 mm instrument (MRCA 400/54/ASC, MRY0020874)
Bruker AVANCE III 400 MHz/54 mm Ascend instrument (BZH 993′400′70I, D315′54-9213)
Bruker AVANCE III 400 MHz/54 mm Ascend instrument (BZH 1157′400′70I, D315′54-9574)
Bruker AVANCE III 400 MHz/54 mm Ascend instrument (BZH 1126′400′70I, D315′54-9527)
Bruker AVANCE NEO 400 MHz/54 mm Ascend instrument (BZH 1396′400′70I, D315′54-10089)
Bruker AVANCE NEO 400 MHz/54 mm Ascend instrument (BZH 1373′400′70I, D315′54-10026)
Varian 400MR 400 MHz/54 mm instrument (MRCA 400/54/ASC, 20609)
Varian 400MR 400 MHz/54 mm instrument (MRCA 400/54/ASC, 20188)

The data were processed and analyzed using Topspin 2.1 software. Where the number of protons assigned is less than the theoretical number of protons in the molecule, it is assumed that the apparently missing signal(s) is/are obscured by solvent and/or water peaks. In addition, where spectra were obtained in protic NMR solvents, exchange of NH and/or OH protons with solvent occurs and hence such signals are normally not observed. Chemical shifts are expressed in parts per million (ppm). Coupling constants are in units of hertz (Hz). Splitting patterns describe apparent multiplicities and are designated as s (singlet), d (doublet), t (triplet), q (quartet), quint (quintet), m (multiplet), br (broad). The chemical shifts are referenced to solvent peaks, which in ¹H NMR appear at 7.27 ppm for CDCl₃, 2.50 for DMSO-d₆, 4.79 for D₂O and 3.31 ppm for CD₃OD.

### Analytical LC-MS System and Method Description for Final Products:

In the following examples, compounds were characterized by mass spectroscopy using the systems and operating conditions set out below. Where atoms with different isotopes are present and a single mass quoted, the mass quoted for the compound is the monoisotopic mass (i.e. ³⁵CI; ⁷⁹Br, etc.).
Instrument: Agilent 1200 & 6100B
Scan Mode: Alternating Positive/Negative Electrospray
Scan Range: 100-1000 amu
LC Conditions: The LCMS analysis was conducted on a Kinetex C18 50*2.1 mm column (5 um particles). The gradient employed was:
   Mobile Phase A: Water+0.037% v/v TFA
   Mobile Phase B: Acetonitrile+0.018% v/v TFA

| | | | |
|---|---|---|---|
| Time | % A | % B | Flow Rate |

| | | | | |
|---|---|---|---|---|
| 0.00 | min | 95 | 5 | 1.0 ml/min |
| 0.4 | min | 95 | 5 | 1.0 ml/min |
| 3.0 | min | 5 | 95 | 1.0 ml/min |
| 4.0 | min | 5 | 95 | 1.0 ml/min |

UV detection provided by summed absorbance signal at 214 nm and 254 nm scanning.

### Analytical LC-MS System and Method Description for Intermediate Products:

### Method A:

Instrument: Shimadzu LCMS-2020
Scan Mode: Positive Electrospray Ionization
Scan Range: 100-1000 amu
LC Conditions: The LCMS analysis was conducted on a Luna-C18 2.0*30 mm (3 um particles) column. The gradient employed was:
   Mobile Phase A: Water+0.037% v/v TFA
   Mobile Phase B: Acetonitrile+0.018% v/v TFA

| Time | % A | % B | Flow Rate |
|---|---|---|---|
| 0.00 min | 99.9 | .1 | 0.8 ml/min |
| 0.10 min | 90.0 | 10.0 | 0.8 ml/min |
| 3.50 min | 20.0 | 80.0 | 0.8 ml/min |
| 3.80 min | 90.0 | 10.0 | 1.2 ml/min |
| 4.30 min | 90.0 | 10.0 | 1.2 ml/min |

UV detection provided by summed absorbance signal at 214 nm and 254 nm scanning.

### Method B:

Instrument: Agilent 1200 & 6110B
Scan Mode: Positive Electrospray Ionization
Scan Range: 100-1000 amu
LC Conditions: The LCMS analysis was conducted on a Xbridge Shield RP18 2.1*50 mm, (5 um particles) column. The gradient employed was:
   Mobile Phase A: 10 mM ammonium bicarbonate in water
   Mobile Phase B: Acetonitrile

| Time | % A | % B | Flow Rate |
|---|---|---|---|
| 0.00 min | 90.0 | 10.0 | 1.0 ml/min |
| 2.00 min | 10.0 | 90.0 | 1.0 ml/min |
| 2.48 min | 10.0 | 90.0 | 1.0 ml/min |
| 2.50 min | 90.0 | 10.0 | 1.2 ml/min |
| 3.00 min | 90.0 | 10.0 | 1.2 ml/min |

UV detection provided by summed absorbance signal at 214 nm and 254 nm scanning.

### Preparative LC-MS System and Method Description:

### Method A:

Instrument: Waters Fractionlynx system
Hardware: 2767 Dual Loop Autosampler/Fraction Collector; 2525 preparative pump; CFO (column fluidic organizer) for column selection; RMA (Waters reagent manager) as make up pump; Waters ZQ Mass Spectrometer; Waters 2996 Photo Diode Array detector.

### Waters ZQ Mass Spectrometer:

Scan Mode: Alternating Positive/Negative Electrospray
Scan Range: 100-2000 amu
LC Conditions: Preparative LCMS separations were conducted on a HALO C-18, 4.6*50 mm, 2.7 m, C18 column at 45° C. The gradient employed was:
   Mobile Phase A: Water+0.1% v/v Formic Acid
   Mobile Phase B: Acetonitrile+0.1% v/v Formic Acid

| | | | |
|---|---|---|---|
| Time | % A | % B | Flow Rate |

| | | | | |
|---|---|---|---|---|
| 0.00 | min | 95 | 5 | 1.5 ml/min |
| 1.0 | min | 5 | 95 | 1.5 ml/min |
| 2.0 | min | 5 | 95 | 1.5 ml/min |
| 2.5 | min | 95 | 5 | 1.5 ml/min |
| 3.0 | min | 95 | 5 | 1.5 ml/min |

UV detection provided by summed absorbance signal at 214 nm and 254 nm scanning.

### Method B:

Instrument: Agilent 1100 LC-MS preparative system:
Hardware: 1100 series “prepALS” autosampler; 1100 series “PrepPump” for preparative flow gradient and 1100 series “QuatPump” for pumping modifier in prep flow; 1100 series “MWD” Multi Wavelength Detector; 1100 series “LC-MSD VL” detector; 2× “Prep-FC” fraction collector; “Waters RMA” make-up pump; Agilent Active Splitter.
Agilent MS running conditions: Capillary voltage: 4000 V (3500 V on ES Negative);
Fragmentor/Gain: 150/1; Drying gas flow: 12.0 L/min; Gas Temperature: 350° C.;
Nebuliser Pressure: 50 psig; Scan Range: 125-800 amu; Ionisation Mode: ElectroSpray Positive or ElectroSpray Negative.
LC Conditions: Preparative LCMS separations were conducted on a HALO C-18, 4.6*50 mm, 2.7 m, C18 column at 45° C. The gradient employed was:
   Mobile Phase A: Water+0.1% v/v Formic Acid
   Mobile Phase B: Acetonitrile+0.1% v/v Formic Acid

| | | | |
|---|---|---|---|
| Time | % A | % B | Flow Rate |

| | | | | |
|---|---|---|---|---|
| 0.00 | min | 95 | 5 | 1.5 ml/min |
| 1.0 | min | 5 | 95 | 1.5 ml/min |
| 2.0 | min | 5 | 95 | 1.5 ml/min |
| 2.5 | min | 95 | 5 | 1.5 ml/min |
| 3.0 | min | 95 | 5 | 1.5 ml/min |

UV detection provided by summed absorbance signal at 214 nm and 254 nm scanning.

### Normal Phase Chromatographic Separations:

Silica gel chromatography was performed on Biotage instruments using pre-packaged disposable SiO₂ stationary phase columns with eluent flow rate range of 15 to 200 mL/min, UV detection (254 and 280 nm).

### Schemes and Experimental Procedures

The following schemes and procedures illustrate how compounds of the present invention can be prepared. The specific solvents and reaction conditions referred to are also illustrative and are not intended to be limiting. Compounds not described are either commercially available or are readily prepared by one skilled in the art using available starting materials. The Examples disclosed herein are for illustrative purposes only and are not intended to limit the compounds of the scope of the invention.

I. Preparation of bis-Phenol Starting Materials:

### Synthesis of bis-Phenol-045, [1,1′-biphenyl]-3,3′-diol

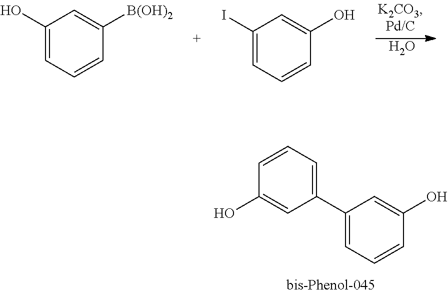 [SMILES: OB(O)c1cccc(O)c1 ; Oc1cccc(I)c1 ; O ; Oc1cccc(-c2cccc(O)c2)c1]

To a solution of (3-hydroxyphenyl)boronic acid (658 mg, 4.77 mmol, 1.05 eq), 3-iodophenol (1.00 g, 4.55 mmol, 1 eq), and K₂CO₃ (2.51 g, 18.2 mmol, 4 eq) in H₂O (50 mL) was added Pd/C (300 mg, 10% purity) in one portion at 15° C. under N₂. The mixture was stirred at 80° C. for 2 h under N₂. LCMS indicated the phenol was completely consumed and the desired mass was detected. The mixture was filtered and the filtrate was acidified by addition of aqueous HCl (1.0 M) to pH=4-5. The mixture was extracted with EtOAc (50 mL*3) and the combined organic phases were washed with brine (30 mL), dried over Na₂SO₄, filtered, and concentrated under reduced pressure to give the title compound (897 mg, 4.82 mmol, quantitative yield) as a yellow oil.

LCMS (ES, m/z): 187.0 [M+H]⁺.

### Synthesis of bis-Phenol-046, [1,1′-biphenyl]-3,4′-diol

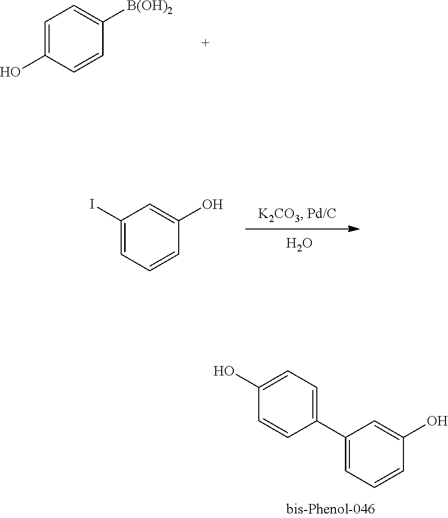 [SMILES: OB(O)c1ccc(O)cc1 ; Oc1cccc(I)c1 ; O ; Oc1ccc(-c2cccc(O)c2)cc1]

To a solution of (4-hydroxyphenyl)boronic acid (1.32 g, 9.55 mmol, 1.05 eq), 3-iodophenol (2 g, 9.09 mmol, 1 eq), and K₂CO₃ (5.03 g, 36.4 mmol, 4 eq) in H₂O (100 mL) was added Pd/C (300 mg, 10% purity) in one portion at 15° C. under N₂. The mixture was stirred at 80° C. for 2 h. LCMS showed indicated the phenol was completely consumed and the desired mass was detected. The mixture was filtered and the filtrate was acidified by addition of aqueous HCl (1.0 M) to pH=4-5. The mixture was extracted with EtOAc (50 mL*3) and the combined organic phases were washed with brine (30 mL), dried over Na₂SO₄, filtered, and concentrated under reduced pressure to give the title compound (1.9 g, 10.2 mmol, quantitative yield) as a brown solid.

LCMS (ES, m/z): 185.0 [M−H]⁻.

### Synthesis of bis-Phenol-055, 1,2-bis(4-hydroxyphenyl)ethane-1,2-dione

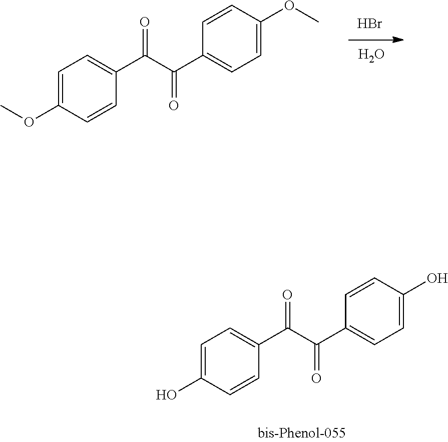

1,2-bis(4-methoxyphenyl)ethane-1,2-dione (2.00 g, 7.40 mmol, 1 eq) was dissolved in HBr/AcOH (40 mL, 33% HBr) and H₂O (15 mL). The reaction mixture was stirred at 140° C. for 16 h after which time LCMS indicated the reaction was complete. The mixture was diluted with water (100 mL) and extracted with ethyl acetate (60 mL*3). The organic phases were combined, washed with water (50 mL*3), dried over Na₂SO₄, and filtered. The filtrate was concentrated under vacuum to give the crude title compound (1.6 g, 6.61 mmol, 89% yield) as a red solid.

LCMS (ES, m/z): 241.0 [M−H]⁻.

### Synthesis of bis-Phenol-139, 5-fluorobenzene-1,3-diol

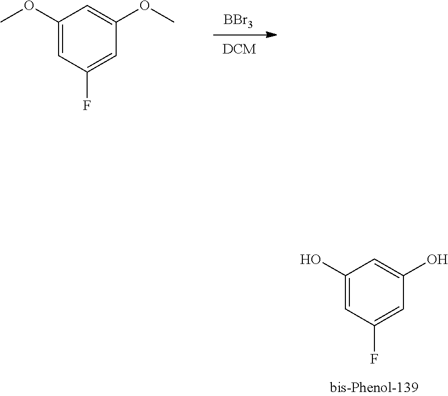

To a solution of 1-fluoro-3,5-dimethoxybenzene (1.50 g, 9.61 mmol, 1 eq) in DCM (50 mL) was added a solution of BBr₃ (5.53 g, 22.1 mmol, 2.13 mL, 2.3 eq) in DCM (50 mL) dropwise over 1.5 h at −65° C. under N₂. The reaction mixture was stirred at 15° C. for 10.5 h after which time LCMS indicated complete consumption of starting material and formation of product of desired mass. The mixture was cooled in an ice/water bath, 60 mL of methanol was added slowly, and the organic solvents were evaporated under vacuum. The residue was partitioned between ethyl acetate and aqueous sodium bicarbonate. The organic phase was isolated, dried over Na₂SO₄, and evaporated to dryness to give the crude product, 5-fluorobenzene-1,3-diol (1.2 g, 9.37 mmol, 97.6% yield), as a red solid. LCMS (ES, m/z): 127.1 [M−H]⁻.

### Synthesis of bis-Phenol-052, 4,4′-(ethane-1,2-diyl)diphenol

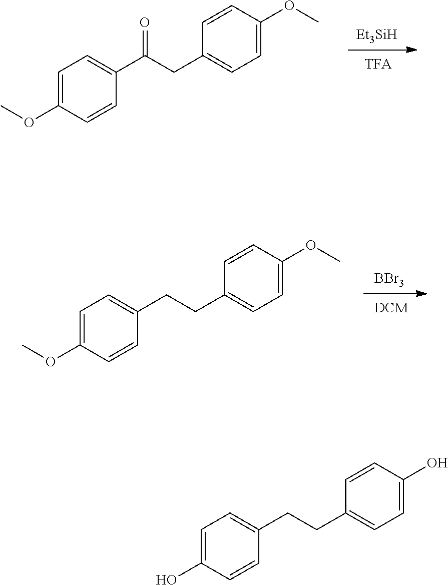

### Step 1 of 2: Synthesis of 1,2-bis(4-methoxyphenyl)ethane

To a solution of 1,2-bis(4-methoxyphenyl)ethan-1-one (5 g, 19.5 mmol, 1 eq) in TFA (20 mL) was added Et₃SiH (11.3 g, 97.5 mmol, 15.6 mL, 5 eq). The mixture was stirred at 20° C. for 12 h. TLC (Petroleum ether/Ethyl acetate=5:1, R_{f}=0.88) showed the reaction was completed. The resulting mixture was diluted with water (200 mL) and extracted with EtOAc (80 mL*3). The combined organic layers were washed with satd. NaHCO₃ aq. until pH=8~9. The organic layer was dried over anhydrous Na₂SO₄, filtered, and concentrated to afford a solid and oil mixture. This material was triturated with petroleum ether (50 mL), filtered and dried to give the title compound (4.4 g, 18.2 mmol, 93% yield) as a white solid.

¹H NMR (400 MHz, CHLOROFORM-d) 6=7.11-6.92 (m, 4H), 6.79-6.63 (m, 4H), 3.70 (s, 6H), 2.75 (s, 4H).

### Step 2 of 2: Synthesis of 4,4′-(ethane-1,2-diyl)diphenol

To a solution of 1,2-bis(4-methoxyphenyl)ethane (4.40 g, 18.2 mmol, 1 eq) in DCM (100 mL) was added BBr₃ (18.2 g, 72.6 mmol, 7.00 mL, 4 eq) at 0° C. The mixture was stirred at 20° C. for 16 h after which time LCMS indicated the reaction was complete. The reaction mixture was poured into water (200 mL) with stirring and the separated aqueous layer was extracted with EtOAc (100 mL*3). The combined organic extracts were washed with water (50 mL*3), dried over Na₂SO₄, and the solvent was removed under vacuum to give the title compound (3.84 g, 17.9 mmol, 98.7% yield) as a light yellow solid.

LCMS (ES, m/z): 213.1 [M−H]⁻.

II. Preparation of di-Bromide Starting Materials:

### Synthesis of di-Bromide-190, 1,4-bis(bromomethyl)-2,3,5,6-tetrafluorobenzene

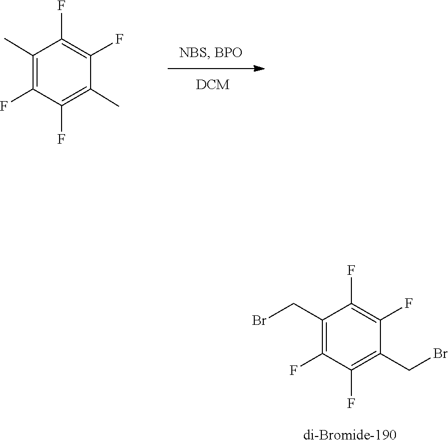

A mixture of 1,2,4,5-tetrafluoro-3,6-dimethylbenzene (0.9 g, 5.05 mmol, 1 eq), NBS (2.70 g, 15.16 mmol, 3 eq) and BPO (184 mg, 758 umol, 0.15 eq) in DCM (40 mL) was stirred at 35° C. for 12 h under irradiation with a 150 W lamp. TLC (Petroleum ether/Ethyl acetate=5:1, R_{f}=0.61) indicated complete consumption of the starting material and formation of a new spot. The reaction mixture was poured into H₂O (50 mL) and extracted with DCM (50 mL*2). The combined organic phases were washed with brine (50 mL), dried over Na₂SO₄, filtered, and concentrated under reduced pressure to give a residue. The residue was purified by column chromatography (SiO₂, Petroleum ether/Ethyl acetate=1:0 to 2:1) to give 1,4-bis(bromomethyl)-2,3,5,6-tetrafluorobenzene (1.40 g, 4.17 mmol, 82.5% yield) as a white solid.

¹H NMR (400 MHz, CHLOROFORM-d) 6=4.52 (s, 4H).

II. Preparation of bis-Amine Linkers, Methods A-M:

### Bis-Amine Linker, Method A:

### Synthesis of Linker A-017, 1,3-phenylenebis(((R)-3-methylpiperazin-1-yl)methanone)

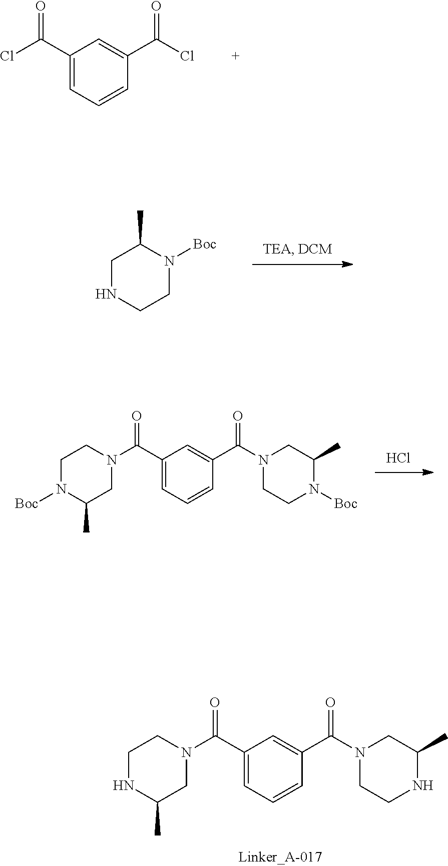 [SMILES: O=C(Cl)c1cccc(C(=O)Cl)c1 ; C[C@@H]1CNCCN1C(=O)OC(C)(C)C ; C[C@@H]1CN(C(=O)c2cccc(C(=O)N3CCN(C(=O)OC(C)(C)C)[C@H](C)C3)c2)CCN1C(=O)OC(C)(C)C ; Cl ; C[C@@H]1CN(C(=O)c2cccc(C(=O)N3CCN[C@H](C)C3)c2)CCN1]

### Step 1 of 2: Synthesis of di-tert-butyl 4,4′-isophthaloyl(2R,2′R)-bis(2-methylpiperazine-1-carboxylate)

To a solution of tert-butyl (R)-2-methylpiperazine-1-carboxylate (1.00 g, 5.20 mmol) in dichloromethane (30 mL) was added triethylamine (1.12 g, 11.1 mmol) followed by isophthaloyl dichloride (0.50 g, 2.5 mmol) slowly. The mixture was stirred at room temperature overnight, concentrated to dryness, and purified by flash chromatography (SiO₂, dichloromethane/methanol=15:1) to give the title compound (1.3 g, 2.4 mmol, 98% yield) as a white solid.

LCMS (ES, m/z): 553.8 [M+Na]⁺.

¹H NMR (400 MHz, DMSO) δ ppm 7.59-7.31 (m, 4H), 4.45-4.00 (m, 4H), 3.95-3.33 (m, 4H), 3.30-2.80 (m, 6H), 1.41 (s, 18H), 1.06 (d, J=48 Hz, 6H).

### Step 2 of 2: Synthesis of 1,3-phenylenebis(((R)-3-methylpiperazin-1-yl)methanone)

To a solution of di-tert-butyl 4,4′-isophthaloyl(2R,2′R)-bis(2-methylpiperazine-1-carboxylate) (1.3 g, 2.4 mmol) in dichloromethane (10 mL) was added HCl (10 mL, 4 M in dioxane). The resulting mixture was stirred at room temperature overnight and concentrated to dryness to give the crude title compound (0.90 g, 2.2 mmol, 91% yield, 2 HCl) as a white solid.

LCMS (ES, m/z): 330.9 [M+H]⁺.

### Synthesis of Linker A-167, 1,3-bis(((R)-3-methylpiperazin-1-yl)sulfonyl)benzene

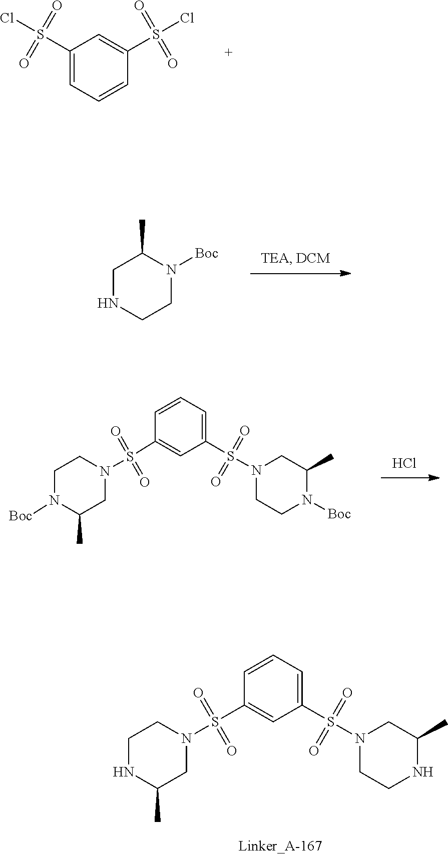 [SMILES: O=S(=O)(Cl)c1cccc(S(=O)(=O)Cl)c1 ; C[C@@H]1CNCCN1C(=O)OC(C)(C)C ; C[C@@H]1CN(S(=O)(=O)c2cccc(S(=O)(=O)N3CCN(C(=O)OC(C)(C)C)[C@H](C)C3)c2)CCN1C(=O)OC(C)(C)C ; Cl ; C[C@@H]1CN(S(=O)(=O)c2cccc(S(=O)(=O)N3CCN[C@H](C)C3)c2)CCN1]

The title compound was prepared according to the two-step procedure of Method A from tert-butyl (R)-2-methylpiperazine-1-carboxylate and benzene-1,3-disulfonyl dichloride.

LCMS (ES, m/z): 402.8 [M+H]⁺.

### Bis-Amine Linker Synthesis, Method B: Synthesis of Linker D-192, (2S,2'S)-2,2′-(((5-methyl-1,3-phenylene)bis(oxy))bis(methylene))dimorpholine

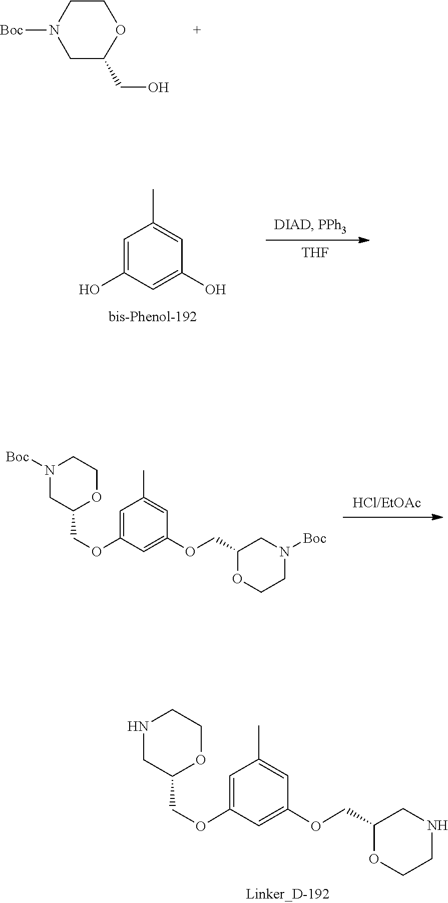

### Step 1 of 2: Synthesis of di-tert-butyl 2,2′-(((5-methyl-1,3-phenylene)bis(oxy))bis(methylene))(2S,2'S)-bis(morpholine-4-carboxylate)

To a mixture of tert-butyl (S)-2-(hydroxymethyl)morpholine-4-carboxylate (500 mg, 2.30 mmol, 2.5 eq), 5-methylbenzene-1,3-diol (114 mg, 920 umol, 1 eq) and PPh₃ (603 mg, 2.30 mmol, 2.5 eq) in THE (4 mL) was added DIAD (465 mg, 2.30 mmol, 447 μL, 2.5 eq) in one portion at 0° C. under N₂. The mixture was stirred at 60° C. for 12 h after which time LCMS indicated complete consumption of the diol and formation of a product of target mass. The mixture was concentrated to dryness and the residue was purified by column chromatography (SiO₂, Petroleum ether/Ethyl acetate=I/O to 0/1). The impure product was repurified by reverse-phase MPLC (40 g ISCO Reverse Phase (C18) flash column; mobile phase: [water-ACN]; B %: 0-55% @30 mL/min) to give the title compound (410 mg, 784 umol, 43% yield) as a white solid.

LCMS (ES, m/z): 423.2 [M-Boc+H⁺]

### Step 2 of 2: Synthesis of (2S,2'S)-2,2′-(((5-methyl-1,3-phenylene)bis(oxy))bis(methylene))dimorpholine

To a solution of di-tert-butyl 2,2′-(((5-methyl-1,3-phenylene)bis(oxy))bis(methylene))(2S,2'S)-bis(morpholine-4-carboxylate) (410 mg, 784 umol, 1 eq) in EtOAc (1.5 mL) was added HCl/EtOAc (4 M, 5 mL, 25 eq). The mixture was stirred at 15° C. for 2 h after which time LCMS indicated complete consumption of starting material and formation of a product of target mass. The reaction mixture was filtered to give the title compound (290 mg, 734 umol, 94% yield, 2 HCl), as a yellow solid.

LCMS (ES, m/z): 323.3 [M+H]⁺.

Bis-Amine Linker synthesis, Method C:

### Synthesis of Linker D-061, 1,4-bis(2-(4-(((S)-morpholin-2-yl)methoxy)phenyl)propan-2-yl)benzene

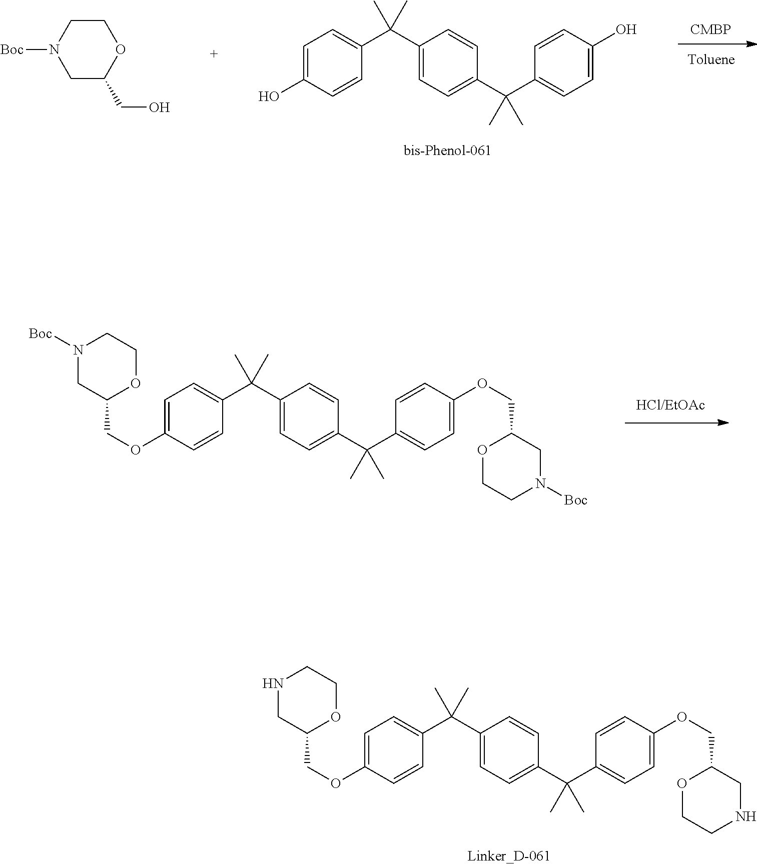 [SMILES: CC(C)(C)OC(=O)N1CCO[C@H](CO)C1 ; CC(C)(c1ccc(O)cc1)c1ccc(C(C)(C)c2ccc(O)cc2)cc1 ; CC(C)(C)OC(=O)N1CCO[C@H](COc2ccc(C(C)(C)c3ccc(C(C)(C)c4ccc(OC[C@@H]5CN(C(=O)OC(C)(C)C)CCO5)cc4)cc3)cc2)C1 ; CC(C)(c1ccc(OC[C@@H]2CNCCO2)cc1)c1ccc(C(C)(C)c2ccc(OC[C@@H]3CNCCO3)cc2)cc1]

### Step 1 of 2: Synthesis of di-tert-butyl 2,2′-((((1,4-phenylenebis(propane-2,2-diyl))bis(4,1-phenylene))bis(oxy))bis(methylene))(2S,2'S)-bis(morpholine-4-carboxylate)

Two reactions were carried out in parallel:

To a mixture of tert-butyl (2S)-2-(hydroxymethyl)morpholine-4-carboxylate (392 mg, 1.80 mmol, 2.5 eq), 4,4′-(1,4-phenylenebis(propane-2,2-diyl))diphenol (250 mg, 722 umol, 1 eq) in toluene (4 mL) was added (Tributylphosphoranylidene)acetonitrile (435 mg, 1.80 mmol, 2.5 eq) at 15° C. under N₂. The mixture was stirred at 80° C. for 12 h under N₂. LCMS showed the diol was completely consumed and a product of desired mass was detected. The two reactions were combined and concentrated in vacuo. The residue was purified by column chromatography (SiO2, Petroleum ether/Ethyl acetate=I/O to 0/1) to give impure product. This material was repurified by reversed-phase MPLC (80 g ISCO Reverse Phase (C18) flash column; mobile phase: [water-ACN]; B %: 0-60% @50 mL/min) to give the title compound (280 mg, 376 umol, 26% yield, impure) as a yellow oil.

TLC (Petroleum ether/Ethyl acetate=2:1): R_{f}=0.33

LCMS (ES, m/z): 767.4 [M+Na]⁺.

### Step 2 of 2: Synthesis of 1,4-bis(2-(4-(((S)-morpholin-2-ylmethoxy)phenyl)propan-2-yl)benzene

To a solution of di-tert-butyl 2,2′-((((1,4-phenylenebis(propane-2,2-diyl))bis(4,1-phenylene))bis(oxy))bis(methylene))(2S,2'S)-bis(morpholine-4-carboxylate) (280 mg, 376 umol, 1 eq) in EtOAc (1 mL), was added HCl/EtOAc (4 M, 2 mL). The mixture was stirred at 15° C. for 2 h. LCMS indicated the starting material was completely consumed and a product of desired mass was detected. The resulting solids were filtered off to give the title compound (220 mg, 356 umol, 95% yield, 2 HCl) as a white solid.

LCMS (ES, m/z): 545.3 [M+H]⁺.

Bis-Amine Linker synthesis, Method D:

### Synthesis of Linker D-147, di-tert-butyl 2,2′-(((1,2-phenylenebis(methylene))bis(oxy))bis(methylene))(2S,2'S)-bis(morpholine-4-carboxylate)

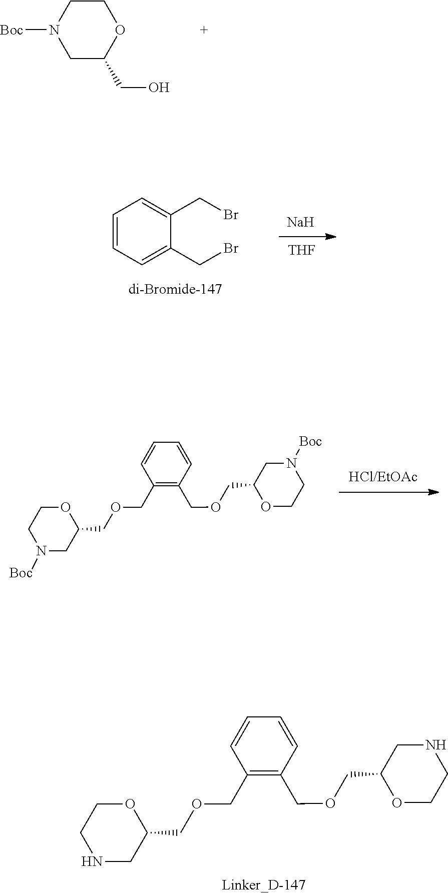

### Step 1 of 2: Synthesis of di-tert-butyl 2,2′-(((1,2-phenylenebis(methylene))bis(oxy))bis(methylene))(2S,2'S)-bis(morpholine-4-carboxylate)

NaH (197 mg, 4.93 mmol, 60% purity, 2.6 eq) was added to a solution of compound tert-butyl (S)-2-(hydroxymethyl)morpholine-4-carboxylate (1.03 g, 4.74 mmol, 2.5 eq) in THE (10 mL) at 0° C. and the mixture was stirred at 0° C. for 0.5 h. 1,2-bis(bromomethyl)benzene (500 mg, 1.89 mmol, 255 μL, 1 eq) was added to the mixture and stirred at 60° C. for 12 h. LCMS showed the starting material was completely consumed and a product of desired mass was detected. H₂O (15 ml) was added to the mixture and the mixture was extracted with EtOAc (15 mL*2). The combined organic layers were washed with brine (15 mL), dried over Na₂SO₄, and filtered. The filtrate was concentrated under reduced pressure to give the title compound (1.3 g, 2.42 mmol, quantitative yield) as a yellow oil.

LCMS (ES, m/z): 559.3 [M+Na]⁺.

### Step 2 of 2: Synthesis of Linker D-147, di-tert-butyl 2,2′-(((1,2-phenylenebis(methylene))bis(oxy))bis(methylene))(2S,2'S)-bis(morpholine-4-carboxylate)

To a solution of di-tert-butyl 2,2′-(((1,2-phenylenebis(methylene))bis(oxy))bis(methylene))(2S,2'S)-bis(morpholine-4-carboxylate) (1 g, 1.86 mmol, 1 eq) in EtOAc (5 mL) was added HCl/EtOAc (4 M, 5 mL) at 15° C. The mixture was stirred at 15 C for 2 h. LCMS showed complete consumption of starting material and formation of a product of desired mass. The mixture was filtered and the filter cake was dried to give the title compound (604 mg, 1.48 mmol, 79% yield, 2 HCl). LCMS (ES, m/z): 337.2 [M+H]⁺.

Bis-Amine Linker synthesis, Method E:

### Synthesis of Linker D-088, 1,6-bis(((S)-morpholin-2-yl)methoxy)hexa-2,4-diyne

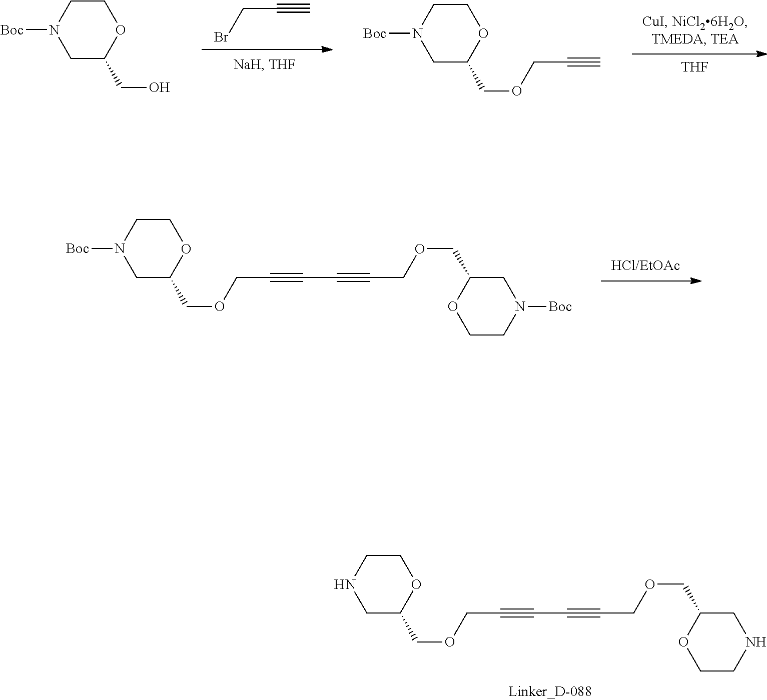

### Step 1 of 3: Synthesis of tert-butyl (S)-2-((prop-2-yn-1-yloxy)methyl)morpholine-4-carboxylate

NaH (276 mg, 6.90 mmol, 60% purity, 1.5 eq) was added to a solution of tert-butyl (S)-2-(hydroxymethyl)morpholine-4-carboxylate (1.00 g, 4.60 mmol, 1 eq) in THE (10 mL) at 0° C. and the mixture was stirred at 15° C. for 0.5 h. 3-Bromoprop-1-yne (1.21 g, 9.20 mmol, 2 eq) was added to the mixture and stirred at 15° C. for 12 h. TLC indicated formation of a new product and LCMS indicated the desired mass was detected. Saturated aqueous NH₄Cl (5 mL) was added to the mixture and the mixture was extracted with EtOAc (5 mL*2). The combined organic layers were washed with brine (10 mL), dried over Na₂SO₄, and filtered. The filtrate was concentrated under reduced pressure to give a residue. The residue was purified by column chromatography (SiO₂, Petroleum ether/Ethyl acetate=20/1 to 5/1) to give the title compound product (0.95 g, 3.72 mmol, 63% yield) as a yellow oil.

TLC (Petroleum ether/Ethyl acetate=5:1): R_{f}=0.43

LCMS (ES, m/z): 156.1 [M-Boc+H]⁺.

### Step 2 of 3: Synthesis of di-tert-butyl 2,2′-((hexa-2,4-diyne-1,6-diylbis(oxy))bis(methylene))(2S,2'S)-bis(morpholine-4-carboxylate)

A mixture of tert-butyl (S)-2-((prop-2-yn-1-yloxy)methyl)morpholine-4-carboxylate (450 mg, 1.76 mmol, 1 eq), CuI (67 mg, 352 umol, 0.2 eq), NiCl₂·6H₂O (84 mg, 353 umol, 0.2 eq), TMEDA (82 mg, 705 umol, 106 μL, 0.4 eq) and TEA (892 mg, 8.81 mmol, 1.23 mL, 5 eq) in THE (5 mL) was stirred at 15° C. for 12 h under aerobic conditions. TLC indicated formation of a new product. The reaction was concentrated in vacuo and the residue was purified by column chromatography (SiO₂, Petroleum ether/Ethyl acetate=5:1 to 1:1) to give the title compound (0.76 g, 1.49 mmol, 38% yield) as an oil.

TLC (Petroleum ether/Ethyl acetate=2:1): R_{f}=0.43

¹H NMR (400 MHz, CHLOROFORM-d) 6=4.40-4.18 (m, 4H), 4.01-3.79 (m, 6H), 3.67-3.49 (m, 8H), 3.14-2.55 (m, 4H), 2.07-2.03 (m, 1H), 1.48 (s, 18H).

### Step 3 of 3: Synthesis of Linker D-088, 1,6-bis(((S)-morpholin-2-yl)methoxy)hexa-2,4-diyne

HCl/EtOAc (4 M, 2 mL, 5.35 eq) was added to a solution of di-tert-butyl 2,2′-((hexa-2,4-diyne-1,6-diylbis(oxy))bis(methylene))(2S,2'S)-bis(morpholine-4-carboxylate) (0.76 g, 1.49 mmol, 1 eq) in EtOAc (6 mL) at 15° C. and the mixture was stirred at 15° C. for 12 h. LCMS indicated conversion to a product of target mass. The reaction mixture was filtered and concentrated under reduced pressure to give the title compound (0.27 g, 876 umol, 59% yield) as a white solid.

LCMS (ES, m/z): 309.3 [M+H]⁺.

¹H NMR (400 MHz, METHANOL-d₄) δ=4.88 (s, 14H), 4.38-4.26 (m, 4H), 4.14-4.10 (m, 1H), 4.08 (d, J=3.3 Hz, 1H), 3.94-3.87 (m, 2H), 3.86-3.78 (m, 2H), 3.68-3.58 (m, 4H), 3.36-3.32 (m, 2H), 3.29-3.25 (m, 2H), 3.19-3.11 (m, 2H), 3.04 (br t, J=12.0 Hz, 2H).

The following bis-Amine linkers were prepared from tert-butyl (S)-2-(hydroxymethyl)morpholine-4-carboxylate according to Methods B, C, D, or E as indicated in the Table 4:

TABLE 4

| bis- | | | | Starting Material ID | |
|---|---|---|---|---|---|
| Amine | | Exact | LCMS | (CAS # or | |
| Linker | Structure | Mass | (ES, m/z) | Reference Code) | Method |
| D-044 | 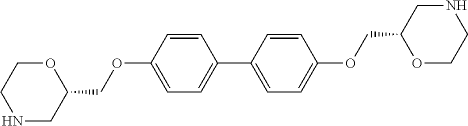 [SMILES: c1cc(-c2ccc(OC[C@@H]3CNCCO3)cc2)ccc1OC[C@@H]1CNCCO1] | 384.20 | 385.2 [M + H]⁺ | 92-88-6 | B |
| D-045 | 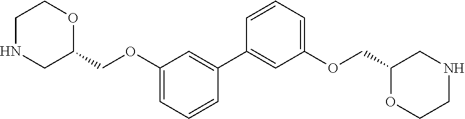 [SMILES: c1cc(OC[C@@H]2CNCCO2)cc(-c2cccc(OC[C@@H]3CNCCO3)c2)c1] | 384.20 | 385.1 [M + H]⁺ | 612-76-0, bis- Phenol- 045 | B |
| D-046 | 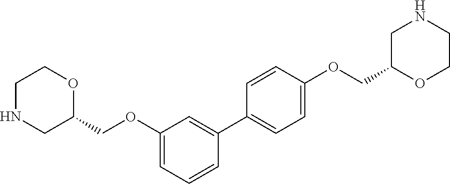 [SMILES: c1cc(OC[C@@H]2CNCCO2)cc(-c2ccc(OC[C@@H]3CNCCO3)cc2)c1] | 384.20 | 385.2 [M + H]⁺ | 18855-13-5, bis- phenol- 046 | B |
| D-047 | 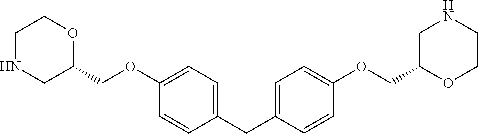 [SMILES: c1cc(OC[C@@H]2CNCCO2)ccc1Cc1ccc(OC[C@@H]2CNCCO2)cc1] | 398.22 | 399.2 [M + H]⁺ | 620-92-8 | B |
| D-048 | 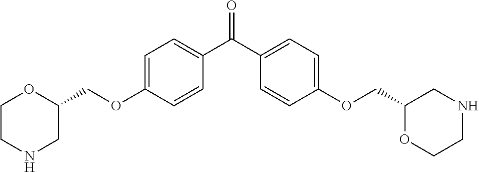 [SMILES: O=C(c1ccc(OC[C@@H]2CNCCO2)cc1)c1ccc(OC[C@@H]2CNCCO2)cc1] | 412.20 | 413.2 [M + H]⁺ | 611-99-4 | B |
| D-049 | 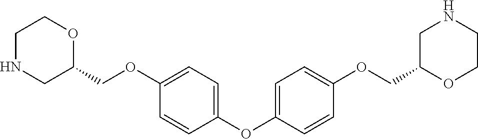 [SMILES: c1cc(Oc2ccc(OC[C@@H]3CNCCO3)cc2)ccc1OC[C@@H]1CNCCO1] | 400.20 | 401.2 [M + H]⁺ | 1965-09-9 | B |
| D-050 | 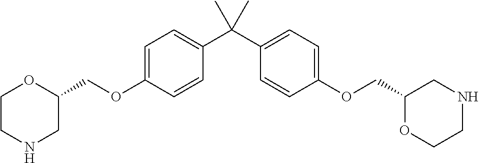 [SMILES: CC(C)(c1ccc(OC[C@@H]2CNCCO2)cc1)c1ccc(OC[C@@H]2CNCCO2)cc1] | 426.25 | 427.2 [M + H]⁺ | 80-05-7 | B |
| D-051 | 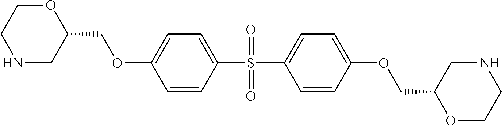 [SMILES: O=S(=O)(c1ccc(OC[C@@H]2CNCCO2)cc1)c1ccc(OC[C@@H]2CNCCO2)cc1] | 448.17 | 449.2 [M + H]⁺ | 80-09-1 | B |
| D-052 | 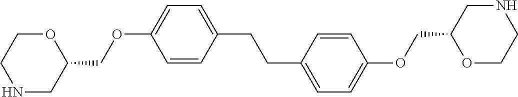 [SMILES: c1cc(OC[C@@H]2CNCCO2)ccc1CCc1ccc(OC[C@@H]2CNCCO2)cc1] | 412.24 | 413.1 [M + H]⁺ | 6052-84-2, bis-phenol- 052 | C |
| D-055 | 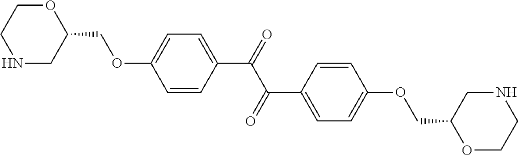 [SMILES: O=C(C(=O)c1ccc(OC[C@@H]2CNCCO2)cc1)c1ccc(OC[C@@H]2CNCCO2)cc1] | 440.19 | 441.1 [M + H]⁺ | 33288-79-8, bis-phenol- 055 | B |
| D-058 | 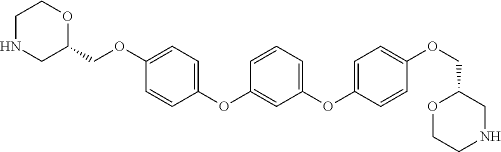 [SMILES: c1cc(Oc2ccc(OC[C@@H]3CNCCO3)cc2)cc(Oc2ccc(OC[C@@H]3CNCCO3)cc2)c1] | 492.23 | 493.2 [M + H]⁺ | 126716-90-3 | B |
| D-059 | 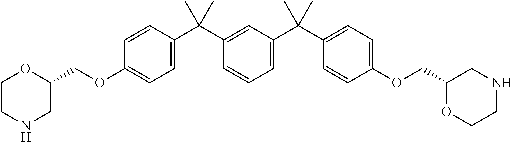 [SMILES: CC(C)(c1ccc(OC[C@@H]2CNCCO2)cc1)c1cccc(C(C)(C)c2ccc(OC[C@@H]3CNCCO3)cc2)c1] | 544.33 | 545.3 [M + H]⁺ | 13595-25-0 | B |
| D-061 | 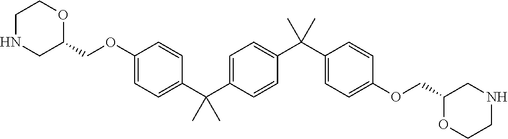 [SMILES: CC(C)(c1ccc(OC[C@@H]2CNCCO2)cc1)c1ccc(C(C)(C)c2ccc(OC[C@@H]3CNCCO3)cc2)cc1] | 544.33 | 545.3 [M + H]⁺ | 2167-51-3 | C |
| D-088 | 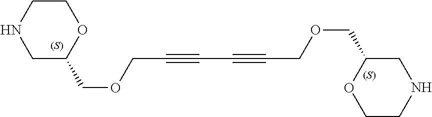 | 308.17 | 309.3 [M + H]⁺ | n/a | E |
| D-089 | 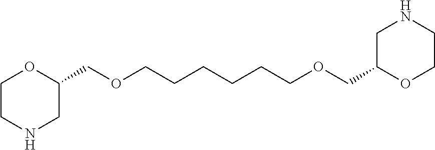 [SMILES: C(CCCOC[C@@H]1CNCCO1)CCOC[C@@H]1CNCCO1] | 316.24 | 317.3 [M + H]⁺ | 629-03-8 | D |
| D-093 | 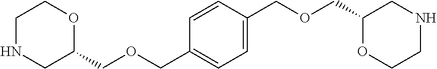 [SMILES: c1cc(COC[C@@H]2CNCCO2)ccc1COC[C@@H]1CNCCO1] | 336.20 | 337.1 [M + H]⁺ | 623-24-5 | D |
| D-120 | 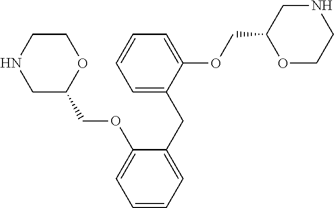 [SMILES: c1ccc(OC[C@@H]2CNCCO2)c(Cc2ccccc2OC[C@@H]2CNCCO2)c1] | 398.22 | 399.2 [M + H]⁺ | 2467-02-9 | B |
| D-121 | 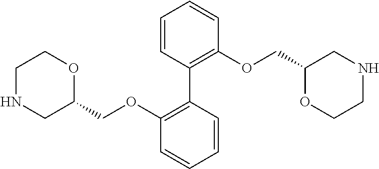 [SMILES: c1ccc(-c2ccccc2OC[C@@H]2CNCCO2)c(OC[C@@H]2CNCCO2)c1] | 384.20 | 385.2 [M + H]⁺ | 1806-29-7 | B |
| D-122 | 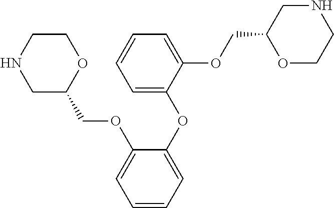 [SMILES: c1ccc(Oc2ccccc2OC[C@@H]2CNCCO2)c(OC[C@@H]2CNCCO2)c1] | 400.20 | 401.2 [M + H]⁺ | 15764-52-0 | B |
| D-131 | 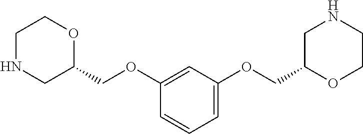 [SMILES: c1cc(OC[C@@H]2CNCCO2)cc(OC[C@@H]2CNCCO2)c1] | 308.17 | 309.2 [M + H]⁺ | 108-46-3 | B |
| D-136 | 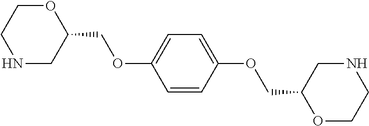 [SMILES: c1cc(OC[C@@H]2CNCCO2)ccc1OC[C@@H]1CNCCO1] | 308.17 | 309.2 [M + H]⁺ | 123-31-9 | C |
| D-137 | 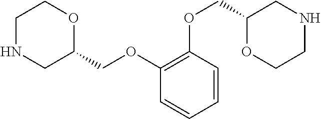 [SMILES: c1ccc(OC[C@@H]2CNCCO2)c(OC[C@@H]2CNCCO2)c1] | 308.17 | 309.2 [M + H]⁺ | 120-80-9 | C |
| D-138 | 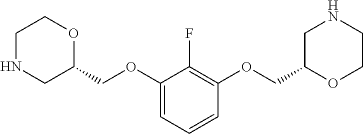 [SMILES: Fc1c(OC[C@@H]2CNCCO2)cccc1OC[C@@H]1CNCCO1] | 326.16 | 327.2 [M + H]⁺ | 103068-40-2 | B |
| D-139 | 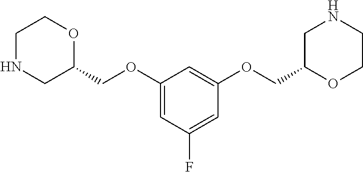 [SMILES: Fc1cc(OC[C@@H]2CNCCO2)cc(OC[C@@H]2CNCCO2)c1] | 326.16 | 327.1 [M + H]⁺ | 75996-29-1, bis-phenol- 139 | B |
| D-140 | 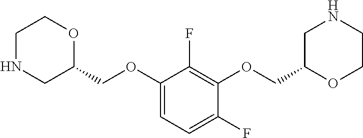 [SMILES: Fc1ccc(OC[C@@H]2CNCCO2)c(F)c1OC[C@@H]1CNCCO1] | 344.15 | 345.2 [M + H]⁺ | 195136-71-1 | B |
| D-141 | 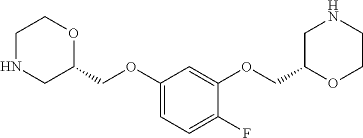 [SMILES: Fc1ccc(OC[C@@H]2CNCCO2)cc1OC[C@@H]1CNCCO1] | 326.16 | 327.1 [M + H]⁺ | 103068-41-3 | B |
| D-142 | 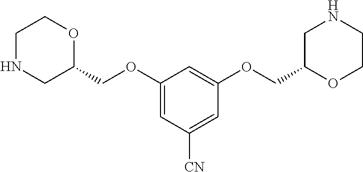 [SMILES: N#Cc1cc(OC[C@@H]2CNCCO2)cc(OC[C@@H]2CNCCO2)c1] | 333.17 | 334.1 [M + H]⁺ | 19179-36-3 | B |
| D-143 | 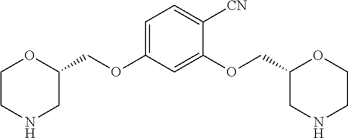 [SMILES: N#Cc1ccc(OC[C@@H]2CNCCO2)cc1OC[C@H]1CNCCO1] | 333.17 | 334.3 [M + H]⁺ | 64419-24-5 | B |
| D-144 | 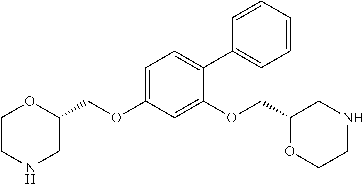 [SMILES: c1ccc(-c2ccc(OC[C@@H]3CNCCO3)cc2OC[C@@H]2CNCCO2)cc1] | 384.20 | 385.1 [M + H]⁺ | 134-52-1 | C |
| D-145 | 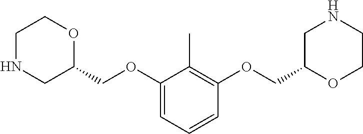 [SMILES: Cc1c(OC[C@@H]2CNCCO2)cccc1OC[C@@H]1CNCCO1] | 322.19 | 323.3 [M + H]⁺ | 608-25-3 | C |
| D-146 | 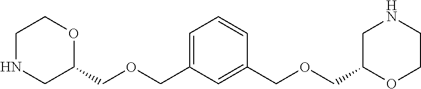 [SMILES: c1cc(COC[C@@H]2CNCCO2)cc(COC[C@@H]2CNCCO2)c1] | 336.20 | 337.2 [M + H]⁺ | 626-15-3 | D |
| D-147 | 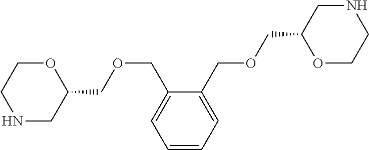 [SMILES: c1ccc(COC[C@@H]2CNCCO2)c(COC[C@@H]2CNCCO2)c1] | 336.20 | 337.2 [M + H]⁺ | 91-13-4 | D |
| D-166 | 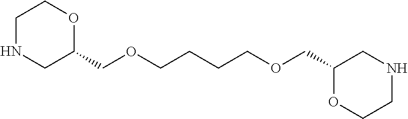 [SMILES: C(CCOC[C@@H]1CNCCO1)COC[C@@H]1CNCCO1] | 288.20 | 289.2 [M + H]⁺ | 110-52-1 | D |
| D-186 | 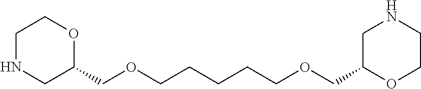 [SMILES: C(CCOC[C@@H]1CNCCO1)CCOC[C@@H]1CNCCO1] | 302.22 | 303.2 [M + H]⁺ | 111-24-0 | D |
| D-188 | 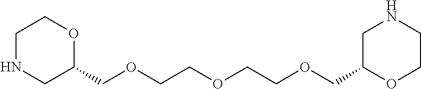 [SMILES: C1CO[C@H](COCCOCCOC[C@@H]2CNCCO2)CN1] | 304.20 | 305.2 [M + H]⁺ | 5414-19-7 | D |
| D-189 | 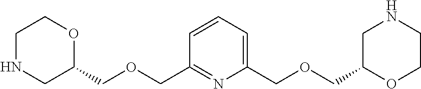 [SMILES: c1cc(COC[C@@H]2CNCCO2)nc(COC[C@@H]2CNCCO2)c1] | 336.20 | 337.1 [M + H]⁺ | 7703-74-4 | D |
| D-190 | 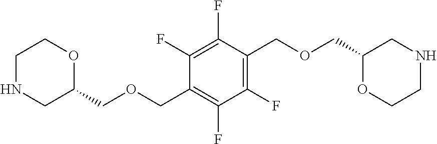 [SMILES: Fc1c(F)c(COC[C@@H]2CNCCO2)c(F)c(F)c1COC[C@@H]1CNCCO1] | 408.17 | 409.2 [M + H]⁺ | di-Bromide- 190 | D |
| D-192 | 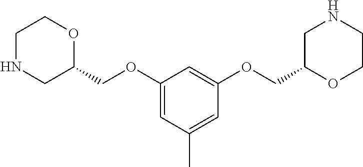 [SMILES: Cc1cc(OC[C@@H]2CNCCO2)cc(OC[C@@H]2CNCCO2)c1] | 322.19 | 323.3 [M + H]⁺ | 504-15-4 | B |
| D-194 | 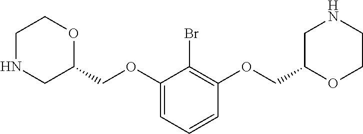 [SMILES: Brc1c(OC[C@@H]2CNCCO2)cccc1OC[C@@H]1CNCCO1] | 386.08, 388.08 | 389.2 [M + H]⁺ | 6751-75-3 | B |
| D-195 | 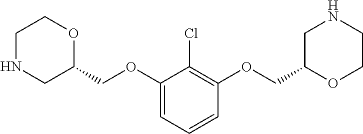 [SMILES: Clc1c(OC[C@@H]2CNCCO2)cccc1OC[C@@H]1CNCCO1] | 342.13 | 343.2 [M + H]⁺ | 6201-65-6 | B |
| D-198 | 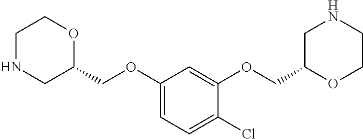 [SMILES: Clc1ccc(OC[C@@H]2CNCCO2)cc1OC[C@@H]1CNCCO1] | 342.13 | 343.2 [M + H]⁺ | 95-88-5 | B |

Bis-Amine Linker synthesis, Method F:

### Synthesis of Linker E-079, (2S,2'S)-N,N′-(1,4-phenylenebis(methylene))bis(morpholine-2-carboxamide)

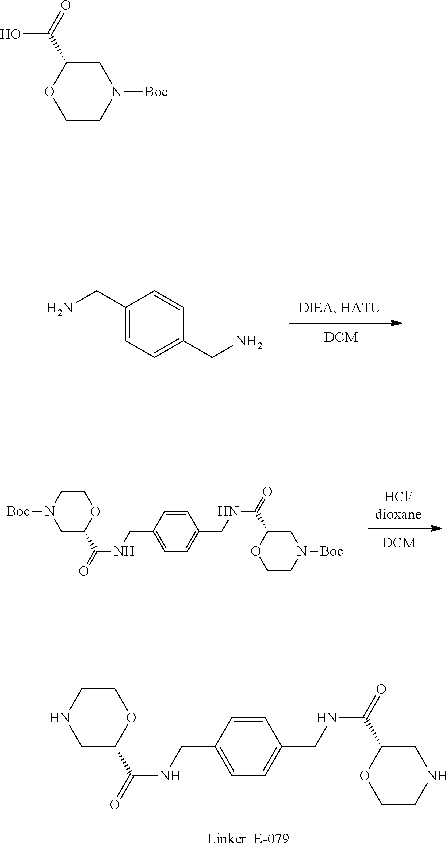 [SMILES: CC(C)(C)OC(=O)N1CCO[C@H](C(=O)O)C1 ; NCc1ccc(CN)cc1 ; CC(C)(C)OC(=O)N1CCO[C@H](C(=O)NCc2ccc(CNC(=O)[C@@H]3CN(C(=O)OC(C)(C)C)CCO3)cc2)C1 ; O=C(NCc1ccc(CNC(=O)[C@@H]2CNCCO2)cc1)[C@@H]1CNCCO1]

### Step 1 of 2: Synthesis of di-tert-butyl 2,2′-(((1,4-phenylenebis(methylene))bis(azanediyl))bis(carbonyl))(2S,2'S)-bis(morpholine-4-carboxylate)

To a solution of (S)-4-(tert-butoxycarbonyl)morpholine-2-carboxylic acid (2.00 g, 8.65 mmol, 2.2 eq), 1,4-phenylenedimethanamine (535 mg, 3.93 mmol, 1 eq) in DCM (20 mL) was added D1EA (2.03 g, 15.7 mmol, 2.74 mL, 4 eq) and HATU (3.74 g, 9.83 mmol, 2.5 eq). The mixture was stirred at 20° C. for 12 h after which time LC-MS indicated conversion to a product of target mass. The reaction mixture was poured into saturated aqueous NH₄Cl (100 mL) and extracted with DCM (50 mL*3). The combined organic phases were washed with brine (100 mL), dried over Na₂SO₄, filtered, and concentrated under reduced pressure to give a residue. The residue was triturated with MeOH (20 ml) and H₂O (20 ml) at 15° C. for 30 min. The mixture was filtered and the filter cake was dried to give the title compound (2.2 g, 3.91 mmol, 99% yield) as a white solid.

LCMS (ES, m/z): 463.2 [M-Boc+H]⁺.

¹H NMR (400 MHz, DMSO-d₆) δ=8.37 (br t, J=6.2 Hz, 2H), 7.16 (s, 4H), 4.24 (br d, J=6.0 Hz, 4H), 4.00 (br d, J=11.6 Hz, 2H), 3.89 (dd, J=3.1, 10.4 Hz, 4H), 3.71 (br d, J=13.0 Hz, 2H), 3.48 (dt, J=2.5, 11.4 Hz, 2H), 3.03-2.70 (m, 4H), 1.41 (s, 18H).

### Step 2 of 2: Synthesis of (2S,2'S)-N,N′-(1,4-phenylenebis(methylene))bis(morpholine-2-carboxamide)

To a mixture of di-tert-butyl 2,2′-(((1,4-phenylenebis(methylene))bis(azanediyl))bis(carbonyl))(2S,2'S)-bis(morpholine-4-carboxylate) (0.20 g, 355 umol, 1 eq) in DCM (4 mL) was added HCl/dioxane (4 M, 4 mL) at 15° C. The mixture was stirred at 15° C. for 1 hr. LCMS indicated consumption of the starting material and formation of a product of desired mass. The mixture was filtered and the filter cake was dried to give the title compound (168 mg, 386 umol, quantitative yield, 2 HCl) as a white solid.

LCMS (ES, m/z): 363.2 [M+H]⁺.

The following bis-amine linkers were prepared from (2S)-4-tert-butoxycarbonylmorpholine-2-carboxylic acid and a diamine according to Method F as indicated in the Table 5:

TABLE 5

| bis- | | | | Starting |
|---|---|---|---|---|
| Amine | | Exact | LCMS (ES, | Material |
| Linker | Structure | Mass | m/z) | (CAS #) |
| E-063 | 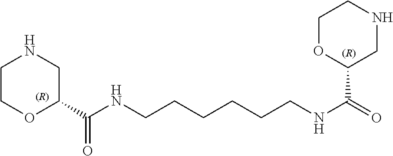 | 342.23 | 342.9 [M + H]⁺ | 124-09-4 |
| E-079 | 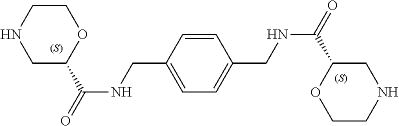 | 362.20 | 363.2 [M + H]⁺ | 539-48-0 |
| E-165 | 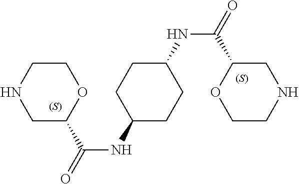 | 340.21 | — | 539-48-0 |

Bis-Amine Linker synthesis, Method G:

### Synthesis of Linker E-182, (2S,2'S)-N,N′-(hexa-2,4-diyne-1,6-diyl)bis(morpholine-2-carboxamide)

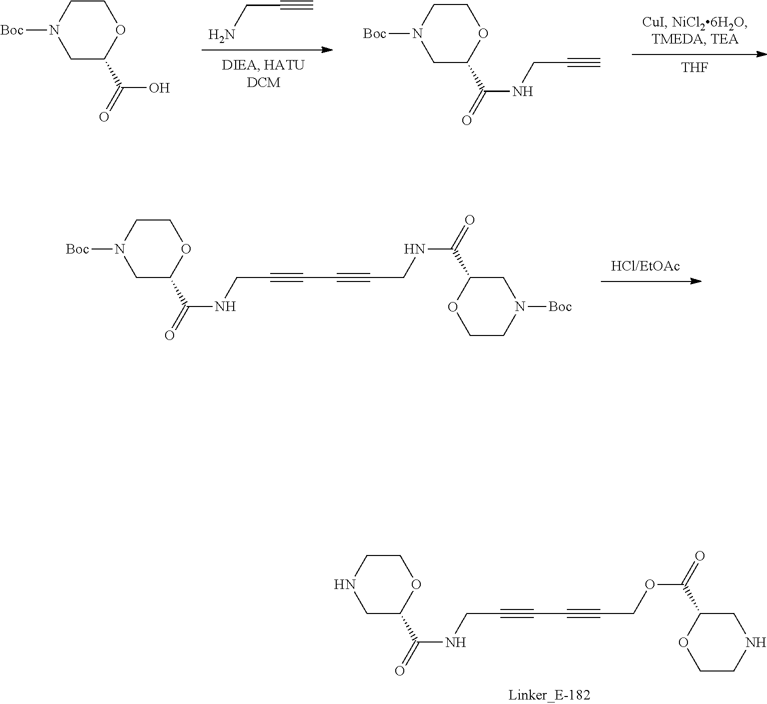

### Step 1 of 3: Synthesis of tert-butyl (S)-2-(prop-2-yn-1-ylcarbamoyl)morpholine-4-carboxylate

To a solution of (2S)-4-tert-butoxycarbonylmorpholine-2-carboxylic acid (2.00 g, 8.65 mmol, 1 eq) and prop-2-yn-1-amine (524 mg, 9.51 mmol, 609 μL, 1.1 eq) in DCM (20 mL) was added DIEA (3.35 g, 25.9 mmol, 4.52 mL, 3 eq) and HATU (4.28 g, 11.2 mmol, 1.3 eq). The mixture was stirred at 20° C. for 12 h after which time a product of desired mass was detected by LC-MS. The mixture was concentrated under reduced pressure and the residue was purified by column chromatography (SiO₂, Petroleum ether/Ethyl acetate=I/O to 0/1) to give the title compound (2.59 g, 9.65 mmol, quantitative yield) as a yellow solid. TLC (Petroleum ether/Ethyl acetate=1:1) R_{f}=0.53

### Step 2 of 3. Synthesis of di-tert-butyl 2,2′-((hexa-2,4-diyne-1,6-diylbis(azanediyl))bis(carbonyl))(2S,2'S)-bis(morpholine-4-carboxylate)

A mixture of tert-butyl (S)-2-(prop-2-yn-1-ylcarbamoyl)morpholine-4-carboxylate (1.00 g, 3.73 mmol, 1 eq), CuI (71 mg, 373 umol, 0.1 eq), NiCl₂·6H₂O (89 mg, 373 umol, 0.1 eq), TMEDA (86.6 mg, 745 umol, 112 μL, 0.2 eq) and TEA (1.89 g, 18.6 mmol, 2.59 mL, 5 eq) in THE (10 mL) was stirred at 15° C. for 12 h under aerobic conditions. LC-MS showed consumption of starting material and formation of a product of target mass. Water (30 ml) was added to the mixture and the mixture was extracted with EtOAc (50 mL*2). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=1:0 to 0:1) to give the title compound (690 mg, 1.29 mmol, 35% yield) as a yellow solid.

TLC (Petroleum ether/Ethyl acetate=1:1): R_{f}=0.24

LCMS (ES, m/z): 435.2 [M-Boc+H]⁺.

¹H NMR (400 MHz, CHLOROFORM-d) 6=6.82-6.69 (m, 2H), 4.41-4.26 (m, 2H), 4.15 (d, J=5.5 Hz, 4H), 3.99-3.87 (m, 5H), 3.65-3.51 (m, 2H), 2.97-2.82 (m, 2H), 2.81-2.66 (m, 2H), 1.61 (s, 1H), 1.52-1.32 (m, 18H).

### Step 3 of 3: Synthesis of Linker E-182, (2S,2'S)-N,N′-(hexa-2,4-diyne-1,6-diyl)bis(morpholine-2-carboxamide)

To a solution of di-tert-butyl 2,2′-((hexa-2,4-diyne-1,6-diylbis(azanediyl))bis(carbonyl))(2S,2'S)-bis(morpholine-4-carboxylate) (200 mg, 374 umol, 1 eq) in EtOAc (2 mL) was added HCl/dioxane (4 M, 2 mL, 21 eq). The mixture was stirred at 20° C. for 0.5 h. LC-MS showed consumption of starting material and formation of a product of target mass. The reaction mixture was filtered and the filtered cake was dried to give the title compound (110 mg, 270 umol, 72.2% yield, 2 HCl) as a white solid.

LCMS (ES, m/z): 335.2 [M+H]⁺.

Bis-Amine Linker synthesis, Method H:

### Synthesis of Linker F-157, N1,N4-bis(2-(piperidin-4-yl)ethyl)terephthalamide

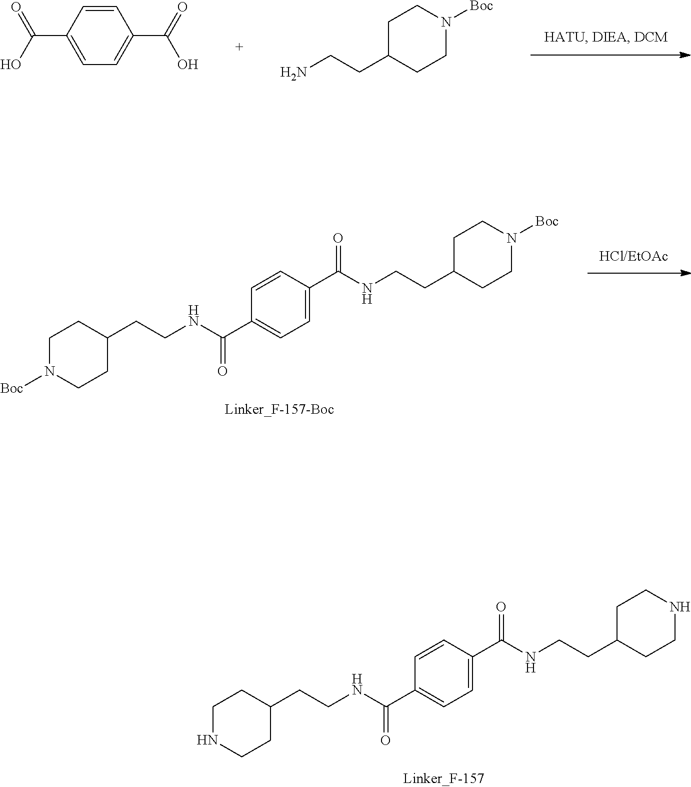 [SMILES: O=C(O)c1ccc(C(=O)O)cc1 ; CC(C)(C)OC(=O)N1CCC(CCN)CC1 ; CC(C)(C)OC(=O)N1CCC(CCNC(=O)c2ccc(C(=O)NCCC3CCN(C(=O)OC(C)(C)C)CC3)cc2)CC1 ; O=C(NCCC1CCNCC1)c1ccc(C(=O)NCCC2CCNCC2)cc1]

### Step 1 of 2: Synthesis of Linker F-157-Boc, di-tert-butyl 4,4′-((terephthaloylbis(azanediyl))bis(ethane-2,1-diyl))bis(piperidine-1-carboxylate)

To a solution of terephthalic acid (661 mg, 3.98 mmol, 1 eq) in DCM (80 mL) were added tert-butyl 4-(2-aminoethyl)piperidine-1-carboxylate (2.00 g, 8.76 mmol, 2.2 eq), DIEA (2.06 g, 15.9 mmol, 2.77 mL, 4 eq) and HATU (3.78 g, 9.95 mmol, 2.5 eq). The mixture was stirred at 15° C. for 12 h. LC-MS showed terephthalic acid was consumed completely and one main peak of desired mass was detected. The mixture was concentrated to dryness and the residue was purified by column chromatography (SiO₂, Ethyl acetate/Methanol=I/O to 3/1) to give the title compound (5.19 g, 8.85 mmol, quantitative yield) as a white solid.

LCMS (ES, m/z): 609.6 [M+Na]⁺.

### Step 2 of 2: Synthesis of Linker F-157, N1,N4-bis(2-(piperidin-4-yl)ethyl)terephthalamide

To a solution of di-tert-butyl 4,4′-((terephthaloylbis(azanediyl))bis(ethane-2,1-diyl))bis(piperidine-1-carboxylate) (1.00 g, 1.70 mmol, 1 eq) in EtOAc (4 mL) was added HCl/EtOAc (4 M, 6 mL). The mixture was stirred at 15° C. for 1 h. LC-MS showed complete consumption of starting material with formation of one main peak of desired mass. The solid was filtered, washed with EtOAc (3*1 mL) and dried under high vacuum to give the title compound (480 mg, 731 umol, 61.3% yield, 2 HCl) as a white solid.

LCMS (ES, m/z): 387.4 [M+H]⁺.

Bis-Amine Linker synthesis, Method I:

### Synthesis of Linker F-154,1,1′-(1,4-phenylene)bis(N-methyl-N-(piperidin-4-ylmethyl)methanamine)

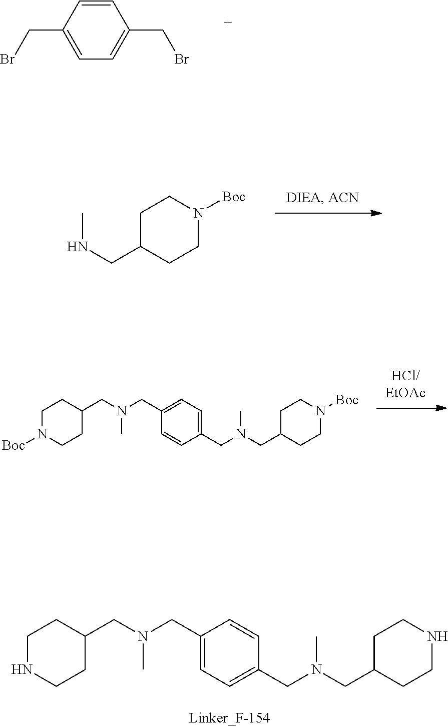 [SMILES: BrCc1ccc(CBr)cc1 ; CNCC1CCN(C(=O)OC(C)(C)C)CC1 ; CN(Cc1ccc(CN(C)CC2CCN(C(=O)OC(C)(C)C)CC2)cc1)CC1CCN(C(=O)OC(C)(C)C)CC1 ; CN(Cc1ccc(CN(C)CC2CCNCC2)cc1)CC1CCNCC1]

### Step 1 of 2: Synthesis of tert-butyl 4-[[[4-[[(1-tert-butoxycarbonyl-4-piperidyl)methyl-methyl-amino]methyl]phenyl]methyl-methyl-amino]methyl]piperidine-1-carboxylate

To a solution of 1,4-bis(bromomethyl)benzene (262 mg, 995 umol, 1 eq) in MeCN (10 mL) was added tert-butyl 4-(methylaminomethyl)piperidine-1-carboxylate (500 mg, 2.19 mmol, 2.2 eq) and DIEA (514 mg, 3.98 mmol, 693 μL, 4 eq). The mixture was stirred at 15° C. for 14 h after which time LC-MS indicated complete consumption of starting material with formation of a product of target mass. The mixture was concentrated under reduced pressure and the residue was purified by column chromatography (SiO₂, Ethyl acetate/Methanol=1:0 to 1:3) to give the title compound (360 mg, 644 umol, 65% yield) as a yellow oil.

TLC (Ethyl acetate/Methanol=10:1): R_{f}=0.4

LCMS (ES, m/z): 559.6 [M+H]⁺.

### Step 2 of 2: Synthesis of N-methyl-1-[4-4[[methyl(4-piperidylmethyl)amino]methyl]phenyl]-N-(4-Piperidylmethyl)methanamine

To a solution of tert-butyl 4-[[[4-[[(1-tert-butoxycarbonyl-4-piperidyl)methyl-methyl-amino]methyl]phenyl]methyl-methyl-amino]methyl]piperidine-1-carboxylate (360 mg, 644 umol, 1 eq) in EtOAc (4 mL) was added HCl/EtOAc (4 M, 4 mL). The mixture was stirred at 15° C. for 1 hr. LC-MS indicated complete consumption of starting material with formation of a product of target mass. The solid was filtered off, washed with EtOAc (3*1 mL), and dried under high vacuum to give the title compound (410 mg, 950 umol, quantitative yield, 2 HCl) as a white solid.

LCMS (ES, m/z): 359.4 [M+H]⁺.

Bis-Amine Linker synthesis, Method J:

### Synthesis of Linker F-158, N1,N4-dimethyl-N1,N4-bis(2-(piperidin-4-yl)ethyl)terephthalamide

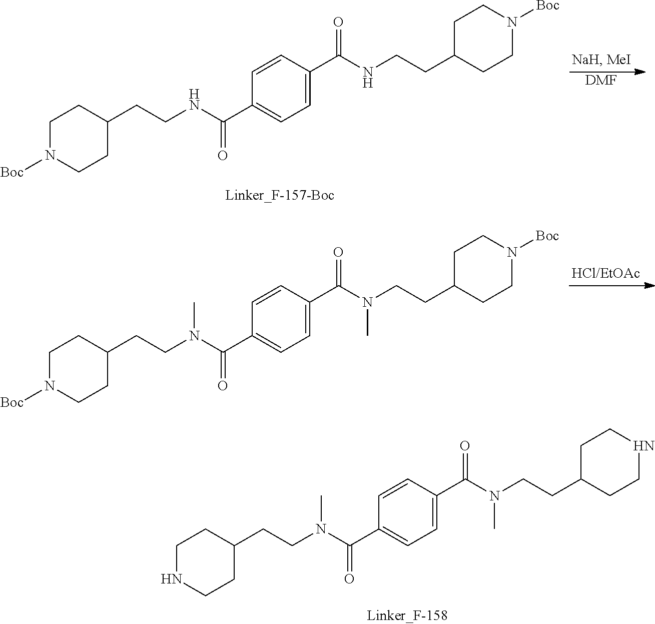

### Step 1 of 2: Synthesis of di-tert-butyl 4,4′-((terephthaloylbis(methylazanediyl))bis(ethane-2,1-diyl))bis(piperidine-1-carboxylate)

To a solution of Linker F-157-Boc, di-tert-butyl 4,4′-((terephthaloylbis(azanediyl))bis(ethane-2,1-diyl))bis(piperidine-1-carboxylate) (1.00 g, 1.70 mmol, 1 eq), in DMF (5 mL) was added NaH (170 mg, 4.26 mmol, 60% purity, 2.5 eq) at 0° C. The mixture was stirred at 0° C. for 0.5 h followed by addition of iodomethane (605 mg, 4.26 mmol, 265 μL, 2.5 eq) at 0° C. The mixture was stirred at 0° C. for 0.5 h followed by 25° C. for 1 hr. LC-MS showed complete consumption of starting material with formation of a single product of target mass. The reaction mixture was quenched with saturated aqueous NH₄Cl (15 mL) and extracted with EtOAc (10 mL*3). The combined organic layers were washed with brine (10 mL), dried over Na₂SO₄, filtered, and concentrated under reduced pressure to give a residue. The residue was purified by column chromatography (SiO₂, Ethyl acetate/Methanol=I/O to 3/1) to give the title compound (165 mg, 268 umol, 16% yield) as a yellow oil.

LCMS (ES, m/z): 637.6 [M+Na]⁺.

### Step 2 of 2: Synthesis of Linker F-158, N1,N4-dimethyl-N1,N4-bis(2-(piperidin-4-yl)ethyl)terephthalamide

To a solution of di-tert-butyl 4,4′-((terephthaloylbis(methylazanediyl))bis(ethane-2,1-diyl))bis(piperidine-1-carboxylate) (165 mg, 268 umol, 1 eq) in EtOAc (2 mL) was added HCl/EtOAc (4 M, 3 mL). The mixture was stirred at 15° C. for 1 h. LC-MS indicated complete consumption of starting material with formation of a single peak of target mass. The solid was filtered, washed with EtOAc (3*1 mL), and dried under high vacuum to give the title compound (117 mg, crude, 2 HCl) as a white solid.

LCMS (ES, m/z): 415.4 [M+H]⁺.

Bis-Amine Linker synthesis, Method K:

### Synthesis of Linker F-159, N,N′-(1,4-phenylenebis(methylene))bis(2-(piperidin-4-yl)acetamide)

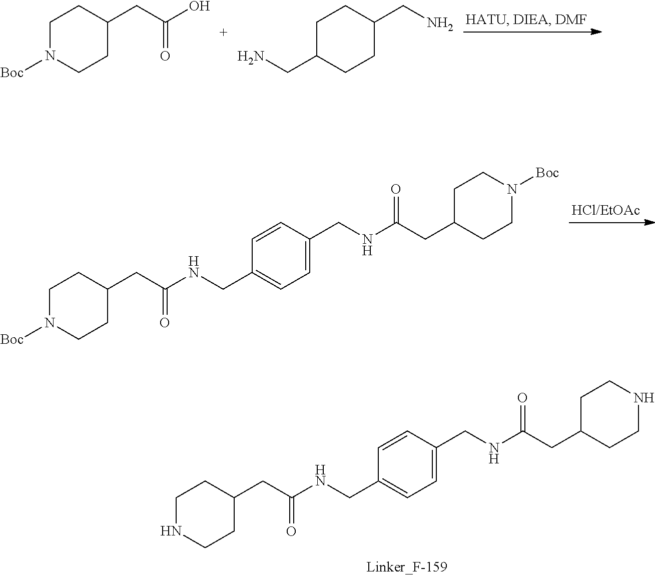

### Step 1 of 2: Synthesis of tert-butyl 4-[2-[[4-[[[2-(1-tert-butoxycarbonyl-4-piperidyl)acetyl]amino]methyl]phenyl]methylamino]-2-oxo-ethyl]piperidine-1-carboxylate

To a solution of 2-(1-tert-butoxycarbonyl-4-piperidyl)acetic acid (2.00 g, 8.22 mmol, 2.2 eq) in DCM (5 mL) were added [4-(aminomethyl)phenyl]methanamine (509 mg, 3.74 mmol, 1 eq), DIEA (1.93 g, 14.9 mmol, 2.60 mL, 4 eq) and HATU (3.55 g, 9.34 mmol, 2.5 eq). The mixture was stirred at 15° C. for 12 hr. LC-MS indicated complete conversion to a product of target mass. The mixture was concentrated under reduced pressure and the residue was purified by column chromatography (SiO₂, Ethyl acetate/Methanol=1:0 to 3:1) to give the title compound (5.49 g, 9.36 mmol, quantitative yield) as a white solid.

TLC (Ethyl acetate/Methanol=10:1): R_{f}=0.4

LCMS (ES, m/z): 587.6 [M+H]⁺.

### Step 2 of 2: Synthesis of Linker F-159, N,N′-(1,4-phenylenebis(methylene))bis(2-(piperidin-4-yl)acetamide)

To a solution of tert-butyl 4-[2-[[4-[[[2-(1-tert-butoxycarbonyl-4-piperidyl)acetyl]amino]methyl]phenyl]methylamino]-2-oxo-ethyl]piperidine-1-carboxylate (1.00 g, 1.70 mmol, 1 eq) in EtOAc (4 mL) was added HCl/EtOAc (4 M, 6 mL). The mixture was stirred at 15° C. for 1 h after which time LCMS indicated complete conversion to a product of target mass. The solid was filtered, washed with EtOAc (3*1 mL), and dried under high vacuum to give the title compound (440 mg, 670 umol, 56.2% yield, 2 HCl) as a white solid.

LCMS (ES, m/z): 387.4 [M+H]⁺.

Bis-Amine Linker synthesis, Method L:

### Synthesis of Linker F-161, 1,3-bis(piperidin-4-ylmethoxy)benzene

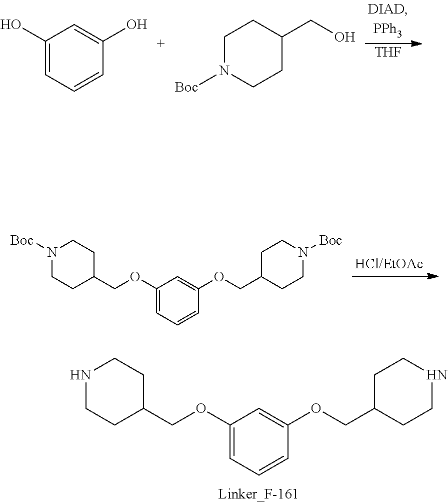

### Step 1 of 2: Synthesis of di-tert-butyl 4,4′-((1,3-phenylenebis(oxy))bis(methylene))bis(piperidine-1-carboxylate)

DIAD (939 mg, 4.64 mmol, 903 μL, 2.5 eq) was added to a mixture of benzene-1,3-diol (204 mg, 1.86 mmol, 310 μL, 1 eq), tert-butyl 4-(hydroxymethyl)piperidine-1-carboxylate (1.00 g, 4.64 mmol, 2.5 eq), and PPh3 (1.22 g, 4.64 mmol, 2.5 eq) in THE (15 mL) under N₂ at 0° C. followed by stirring at 60° C. for 12 h. LC-MS indicated complete consumption of starting material with formation of a single peak of target mass. The reaction mixture was quenched with H₂O (15 mL) and extracted with ethyl acetate (15 mL*2). The combined organic layers were washed with brine (30 mL), dried over Na₂SO₄, filtered, and concentrated under reduced pressure to give a residue. The residue was purified by column chromatography (SiO₂, Petroleum ether/Ethyl acetate=1:0 to 3:1) to give the title compound (513 mg, 1.02 mmol, 54.7% yield) as a white solid.

LCMS (ES, m/z): 505.5 [M+H]⁺.

### Step 2 of 2: Synthesis of 1,3-bis(piperidin-4-ylmethoxy)benzene

To a solution of di-tert-butyl 4,4′-((1,3-phenylenebis(oxy))bis(methylene))bis(piperidine-1-carboxylate) (690 mg, 1.37 mmol, 1 eq) in EtOAc (4 mL) was added HCl/EtOAc (4 M, 6 mL, 17.6 eq). The mixture was stirred at 15° C. for 1 h after which time LC-MS indicated complete consumption of starting material with formation of a single peak of target mass. The solid was filtered, washed with EtOAc (3*1 mL), and dried under high vacuum to give the title compound (400 mg, 1.31 mmol, 96.1% yield, 2 HCl) as a white solid.

LCMS (ES, m/z): 305.3 [M+H]⁺.

Bis-Amine Linker synthesis, Method M:

### Synthesis of Linker F-164, 1,3-bis(2,2-difluoro-2-(piperidin-4-yl)ethoxy)benzene

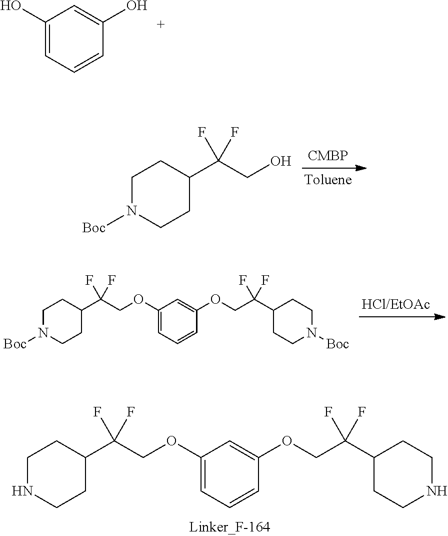

### Step 1 of 2: Synthesis of di-tert-butyl 4,4′-((1,3-phenylenebis(oxy))bis(1,1-difluoroethane-2,1-diyl))bis(piperidine-1-carboxylate)

To a solution of tert-butyl 4-(1,1-difluoro-2-hydroxy-ethyl)piperidine-1-carboxylate (169 mg, 639 umol, 2.2 eq), benzene-1,3-diol (32 mg, 290 umol, 48 μL, 1 eq), in toluene (5 mL) were added (Tributylphosphoranylidene)acetonitrile (175 mg, 726 umol, 2.5 eq) under N₂ atmosphere at 15° C. The mixture was stirred at 90° C. for 12 h after which time LC-MS indicated complete consumption of starting material with formation of a single peak of target mass. The mixture was concentrated to dryness and the residue was purified by column chromatography (SiO₂, Petroleum ether/Ethyl acetate=1:0 to 0:1). The impure product was repurified by preparative HPLC (column: Phenomenex Luna C₁₈ 200*40 mm*10 um; mobile phase: [water (0.2% FA)-ACN]; B %: 60-100%; 8 min) to give the title compound (50 mg, 83 umol, 14% yield) as a yellow oil.

LCMS (ES, m/z): 605.4 [M+H]⁺.

### Step 2 of 2. Synthesis of Linker F-164, 1,3-bis(2,2-difluoro-2-(piperidin-4-yl)ethoxy)benzene

To a solution of di-ter-butyl 4,4′-((1,3-phenylenebis(oxy))bis(1,1-difluoroethane-2,1-diyl))bis(piperidine-1l-carboxylate) (50 mg, 82.7 umol, 1 eq) in EtOAc (1 mL) was added HCl/EtOAc (4 M, 1 mL). The mixture was stirred at 15° C. for 1 h after which time LC-MS indicated complete consumption of starting material with formation of a single peak of target mass. The mixture was filtered and the filter cake was dried to give the title compound (31 mg, 64.9 umol, 78% yield, crude, 2 HCl) as a yellow solid.

LCMS (ES, mSz): 405.2 [M+H]⁺.

The following bis-Amine linkers were prepared according to Methods H, I, J, K, L, or M as indicated in Table 6.

TABLE 6

| | | | | Starting | | |
|---|---|---|---|---|---|---|
| | | | | Material | | |
| bis- | | | LCMS | (Linker | Starting | |
| Amine | | Exact | (ES, | Code | Material | |
| Linker | Structure | Mass | m/z) | or CAS #) | (CAS #) | Method |
| F-149 | 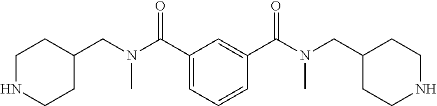 [SMILES: CN(CC1CCNCC1)C(=O)c1cccc(C(=O)N(C)CC2CCNCC2)c1] | 386.27 | 387.3 [M + H]⁺ | 121-91-5 | 138022-02-3 | H |
| F-150 | 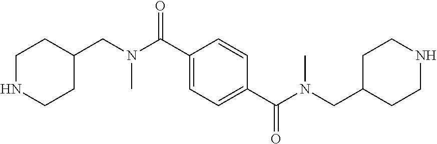 [SMILES: CN(CC1CCNCC1)C(=O)c1ccc(C(=O)N(C)CC2CCNCC2)cc1] | 386.27 | 387.4 [M + H]⁺ | 100-21-0 | 138022-02-3 | H |
| F-151 | 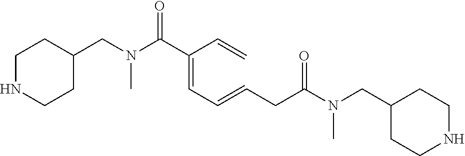 [SMILES: C=C/C(=C\C=C\CC(=O)N(C)CC1CCNCC1)C(=O)N(C)CC1CCNCC1] | 400.28 | 401.4 [M + H]⁺ | 501-89-3 | 138022-02-3 | H |
| F-152 | 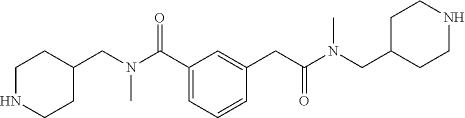 [SMILES: CN(CC1CCNCC1)C(=O)Cc1cccc(C(=O)N(C)CC2CCNCC2)c1] | 400.28 | 401.4 [M + H]⁺ | 2084-13-1 | 138022-02-3 | H |
| F-153 | 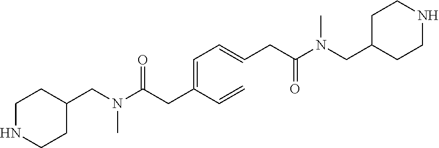 [SMILES: C=C/C(=C\C=C\CC(=O)N(C)CC1CCNCC1)CC(=O)N(C)CC1CCNCC1] | 414.30 | 415.5 [M + H]⁺ | 7325-46-4 | 138022-02-3 | H |
| F-154 | 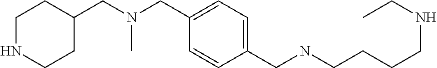 | 358.31 | 359.4 [M + H]⁺ | 623-24-5 | 138022-02-3 | I |
| F-155 | 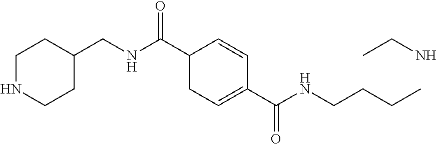 | 358.24 | 359.3 [M + H]⁺ | 100-21-0 | 144222-22-0 | H |
| F-156 | 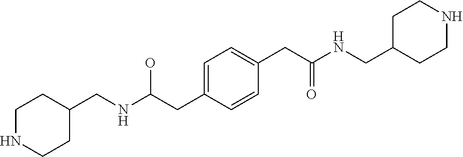 [SMILES: O=C(Cc1ccc(CC(O)NCC2CCNCC2)cc1)NCC1CCNCC1] | 386.27 | 387.4 [M + H]⁺ | 7325-46-9 | 144222-22-0 | H |
| F-157 | 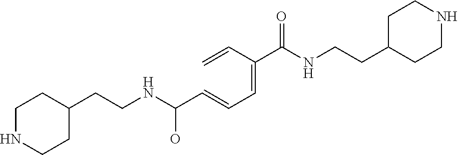 | 386.27 | 387.4 [M + H]⁺ | 100-21-0 | 146093-46-1 | H |
| F-158 | 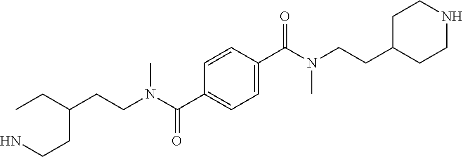 [SMILES: CCC(CCN)CCN(C)C(=O)c1ccc(C(=O)N(C)CCC2CCNCC2)cc1] | 414.30 | 415.4 [M + H]⁺ | Linker_F- 157-Boc | — | J |
| F-159 | 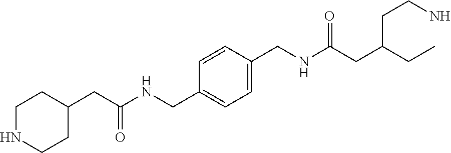 [SMILES: CCC(CCN)CC(=O)NCc1ccc(CNC(=O)CC2CCNCC2)cc1] | 386.27 | 387.4 [M + H]⁺ | 539-48-0 | 157688-46-5 | K |
| F-160 | 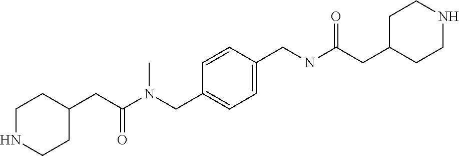 | 414.30 | 415.4 [M + H]⁺ | Linker_F- 159-Boc | — | J |
| F-161 | 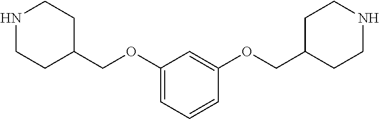 [SMILES: c1cc(OCC2CCNCC2)cc(OCC2CCNCC2)c1] | 304.22 | 305.3 [M + H]⁺ | 123855-51-6 | 108-46-3 | L |
| F-162 | 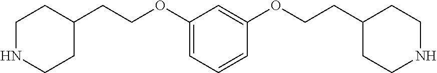 [SMILES: c1cc(OCCC2CCNCC2)cc(OCCC2CCNCC2)c1] | 332.25 | 333.3 [M + H]⁺ | 89151-44-0 | 108-46-3 | L |
| F-163 | 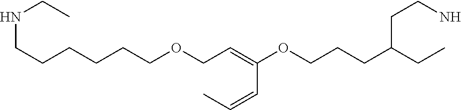 | 360.28 | 361.4 [M + H]⁺ | 156185-63-6 | 108-46-3 | L |
| F-164 | 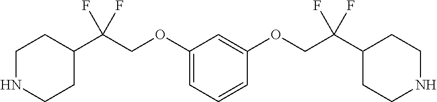 [SMILES: FC(F)(COc1cccc(OCC(F)(F)C2CCNCC2)c1)C1CCNCC1] | 404.21 | 405.2 [M + H]⁺ | 1258639-21-2 | 108-46-3 | M |

### Synthesis of bis-Amine Linker-G-079, (2R,2′R)-N,N′-(1,4-phenylenebis(methylene))bis(morpholine-2-carboxamide)

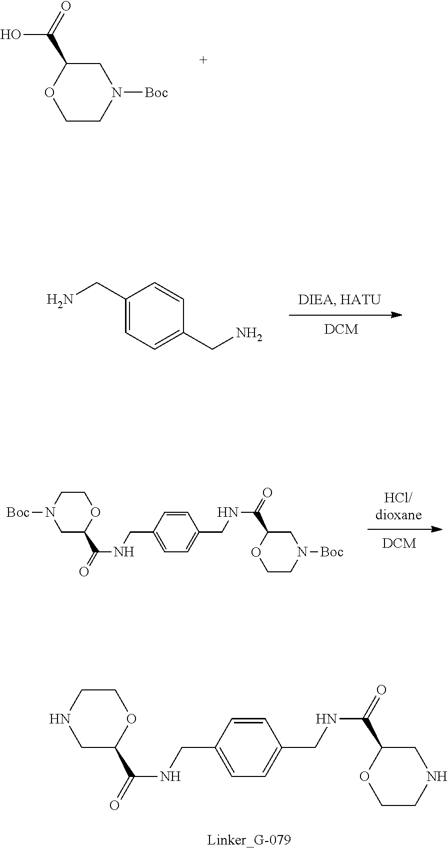 [SMILES: CC(C)(C)OC(=O)N1CCO[C@@H](C(=O)O)C1 ; NCc1ccc(CN)cc1 ; CC(C)(C)OC(=O)N1CCO[C@@H](C(=O)NCc2ccc(CNC(=O)[C@H]3CN(C(=O)OC(C)(C)C)CCO3)cc2)C1 ; O=C(NCc1ccc(CNC(=O)[C@H]2CNCCO2)cc1)[C@H]1CNCCO1]

The title compound was prepared according to the two-step procedure of Method F from (R)-4-(tert-butoxycarbonyl)morpholine-2-carboxylic acid and 1,4-phenylenedimethanamine.

LCMS (ES, m/z): 362.9 [M+H]⁺.

### Synthesis of bis-Amine Linkers 11-131, 1,3-bis(((R)-morpholin-2-yl)methoxy)benzene; and H1-166, 1,4-bis(((R)-morpholin-2-yl)methoxy)butane

Bis-Amine linkers H-131 and H-166 were prepared according to the two step procedures of Methods B and D, respectively, with the exception that tert-butyl (R)-2-(hydroxymethyl)morpholine-4-carboxylate was used instead of tert-butyl (S)-2-(hydroxymethyl)morpholine-4-carboxylate. Characterizing data for these bis-Amines are as shown in Table 7.

TABLE 7

| | | | | Starting | |
|---|---|---|---|---|---|
| | | | | Material | |
| bis- | | | | ID (CAS # or | |
| Amine | | Exact | LCMS (ES, | Reference | |
| Linker | Structure | Mass | m/z) | Code) | Method |
| H-131 | 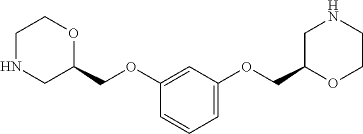 [SMILES: c1cc(OC[C@H]2CNCCO2)cc(OC[C@H]2CNCCO2)c1] | 308.17 | 308.9 [M + H]⁺ | 108-46-3 | B |
| H-166 | 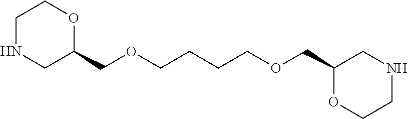 [SMILES: C(CCOC[C@H]1CNCCO1)COC[C@H]1CNCCO1] | 288.20 | 288.9 [M + H]⁺ | 110-52-1 | D |

### IV. Preparation of Key Intermediates:

### Preparation of Intermediate 1.5, tert-butyl (2R,5R)-5-(hydroxymethyl)-2-methylpiperazine-1-carboxylate

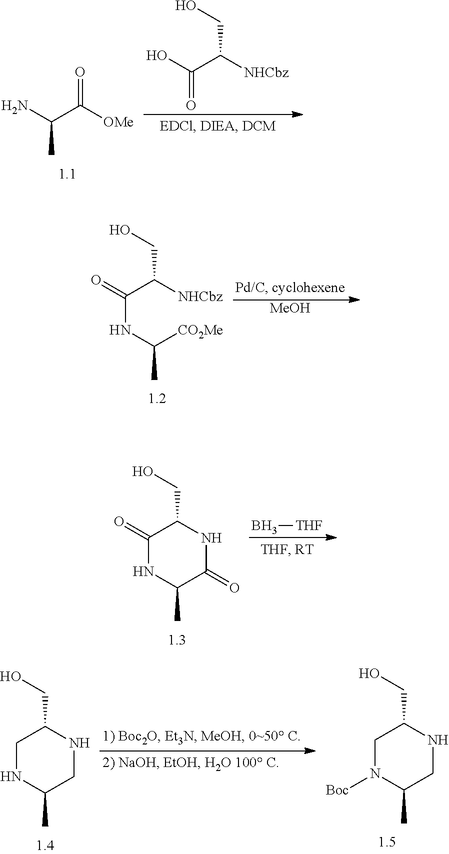

Step 1 of 4: Synthesis of Intermediate 1.2, methyl ((benzyloxy)carbonyl)-L-seryl-D-alaninate.

Two batches were carried out in parallel:

To a solution of methyl D-alaninate (46.7 g, 334.4 mmol, 1 eq, HCl), (2S)-2-(benzyloxycarbonylamino)-3-hydroxy-propanoic acid (80.0 g, 334 mmol, 1 eq), and EDCI (76.9 g, 401 mmol, 1.2 eq) in DCM (800 mL) was added DIEA (129.7 g, 1.00 mol, 174.8 mL, 3 eq). The mixture was stirred at 15° C. for 16 h. The two reactions were combined and concentrated under reduced pressure. The residue was diluted with saturated aqueous NaHCO₃ solution (1000 mL) and H₂O (1000 mL), and then extracted with ethyl acetate (1000 mL*3). The combined organic layers were washed with 2 M HCl (1000 mL) and brine (1000 mL), dried over Na₂SO₄, filtered, and concentrated under reduced pressure to give crude title compound (160 g, 493 mmol, 73.8% yield) as a white solid.

¹H NMR (400 MHz, CHLOROFORM-d) δ ppm 7.27-7.38 (m, 5H) 7.12 (br d, J=5.90 Hz, 1H) 6.02 (br d, J=7.40 Hz, 1H) 5.12 (s, 2H) 4.54 (br t, J=7.34 Hz, 1H) 4.28 (br s, 1H) 4.06 (br d, J=9.41 Hz, 1H) 3.71 (s, 4H) 1.39 (br d, J=6.78 Hz, 3H).

### Step 2 of 4: Synthesis of Intermediate 1.3, (3S,6R)-3-(hydroxymethyl)-6-methylpiperazine-2,5-dione

Four batches were carried out in parallel:

To a solution of methyl ((benzyloxy)carbonyl)-L-seryl-D-alaninate (35 g, 108 mmol, 1 eq) in MeOH (310 mL) was added Pd/C (5 g, 10% purity) and cyclohexene (205 mL). The mixture was stirred at 90° C. for 12 h. The four reactions were combined for work-up. The reaction mixture was filtered and the filter cake was washed with hot MeOH (250*3 mL). The filtrate was concentrated under reduced pressure to give the title compound (51.0 g, 322 mmol, 74.7% yield) as a white solid.

¹H NMR (400 MHz, DMSO-d₆) δ ppm 8.09 (s, 1H) 7.90 (br s, 1H) 5.11 (t, J=5.26 Hz, 1H) 3.92 (d, J=7.02 Hz, 1H) 3.71-3.78 (m, 1H) 3.65-3.71 (m, 1H) 3.52 (ddd, J=10.52, 5.04, 2.85 Hz, 1H) 1.23 (d, J=7.02 Hz, 3H).

### Step 3 of 4: Synthesis of Intermediate 1.4, ((2R,5R)-5-methylpiperazin-2-yl)methanol

Four batches were carried out in parallel:

To a solution of (3S,6R)-3-(hydroxymethyl)-6-methylpiperazine-2,5-dione (12.5 g, 79.0 mmol, 1 eq) in THE (50 mL) was added BH₃·THF (1 M, 474 mL, 474 mmol, 6 eq) at 20° C. The mixture was stirred at 70° C. for 12 h. The four reactions were combined for work-up. The solution was cooled in ice, then methanol (650 ml) was gradually added followed by 5 M HCl (150 ml). The mixture was heated at 70° C. for 2 h and then cooled to 25° C. The solids were filtered off, and the filter cake was washed with THE (250 mL*2) to give the title compound (60 g, 295 mmol, 93% yield, 2 HCl) as a white solid.

¹H NMR (400 MHz, DMSO-d₆) δ ppm 10.05 (br s, 4H) 5.59 (br s, 1H) 3.30-3.86 (m, 6H) 3.10 (q, J=12.28 Hz, 2H) 1.31 (d, J=6.58 Hz, 3H).

### Step 4 of 4: Synthesis of Intermediate 1.5, tert-butyl (2R,5R)-5-(hydroxymethyl)-2-methylpiperazine-1-carboxylate

Three batches were carried out in parallel:

To a solution of ((2R,5R)-5-methylpiperazin-2-yl)methanol (20.0 g, 98.5 mmol, 1 eq, 2 HCl) in MeOH (200 mL) was added Boc₂O (49.4 g, 226 mmol, 52.0 mL, 2.3 eq) and TEA (29.9 g, 295 mmol, 41.1 mL, 3 eq). The mixture was stirred at 50° C. for 12 h. The reaction mixture was filtered and the filtrate was concentrated under reduced pressure to give a residue. The residue was dissolved in EtOH (100 mL) and treated with NaOH (1.5 M, 328 mL, 5 eq) with stirring at 90° C. for 12 h. The three reactions were combined for work-up. The reaction mixture was concentrated under reduced pressure to remove the EtOH, and the pH was adjusted to ~9 with 1 M HCl. The mixture was extracted with DCM (800 mL*6) and the combined organic layers were dried over Na₂SO₄, filtered, and concentrated under reduced pressure to give the title compound as a white solid.

¹H NMR (400 MHz, METHANOL-d₄) δ ppm 4.04-4.16 (m, 1H) 3.76 (dd, J=14.03, 1.75 Hz, 1H) 3.47-3.55 (m, 1H) 3.57-3.66 (m, 1H) 3.47-3.55 (m, 1H) 3.21 (dd, J=14.03, 4.38 Hz, 1H) 3.02 (dd, J=13.15, 4.82 Hz, 1H) 2.86-2.95 (m, 1H) 2.51 (dd, J=12.94, 3.29 Hz, 1H) 1.46 (s, 9H) 1.23 (d, J=7.02 Hz, 3H).

### Preparation of Intermediate 2.2, 2-(4-fluorobenzyl)-4,4,5,5-tetramethyl-1,3,2-dioxaborolane

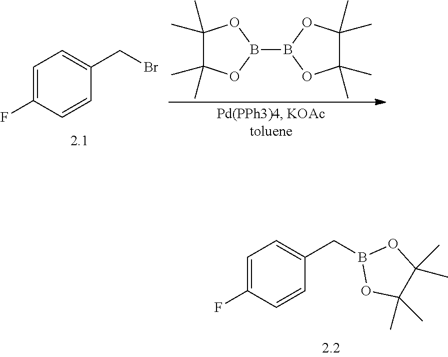

Five batches were carried out in parallel:

Pd(PPh₃)₄(4.58 g, 3.97 mmol, 0.03 eq) was added to a mixture of KOAc (19.5 g, 198 mmol, 1.5 eq), 4,4,5,5-tetramethyl-2-(4,4,5,5-tetramethyl-1,3,2-dioxaborolan-2-yl)-1,3,2-dioxaborolane (36.9 g, 145 mmol, 1.1 eq) and 1-(bromomethyl)-4-fluorobenzene (25.0 g, 132 mmol, 16.3 mL, 1 eq) in toluene (460 mL) under N₂. The mixture was stirred at 100° C. for 3 h. TLC (Petroleum ether/Ethyl acetate=10:1) indicated complete consumption of starting material and formation of new spots. The five reactions were combined for work-up. The reaction mixture was filtered and the mixture was quenched with H₂O (1500 ml) at 0° C. The mixture was extracted with EtOAc (1000 ml*3) and the combined organic phases were washed with brine (500 ml), dried over anhydrous Na₂SO₄, filtered, and concentrated in vacuo. The residue was purified by column chromatography (SiO₂, Petroleum ether/Ethyl acetate=300/1 to 0/1) to give the title compound (127 g, 538 mmol, 55.2% yield) as a yellow liquid.

¹H NMR (400 MHz, CHLOROFORM-d) 6=7.18-7.02 (m, 2H), 6.98-6.88 (m, 2H), 2.32-2.21 (m, 2H), 1.25 (br s, 1H).

### Preparation of Intermediate 3.14, tert-butyl (2R,5R)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-5-(hydroxymethyl)-2-methylpiperazine-1-carboxylate

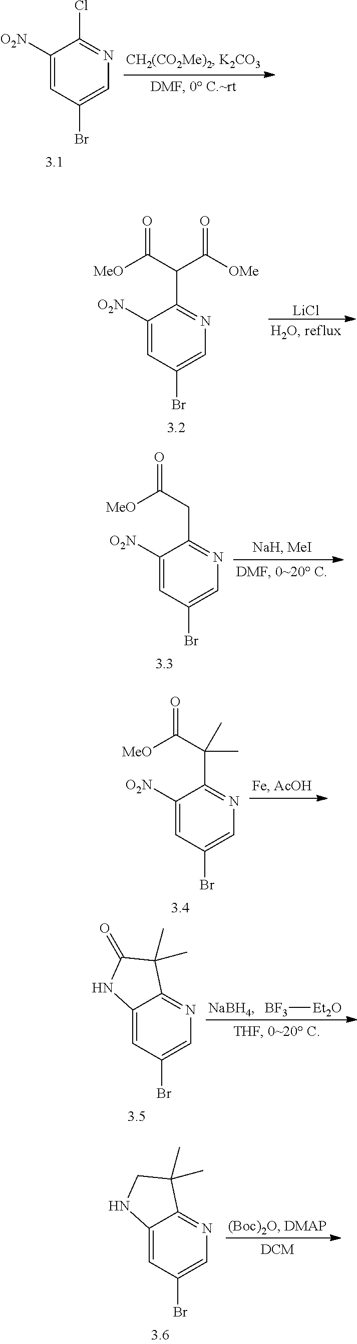

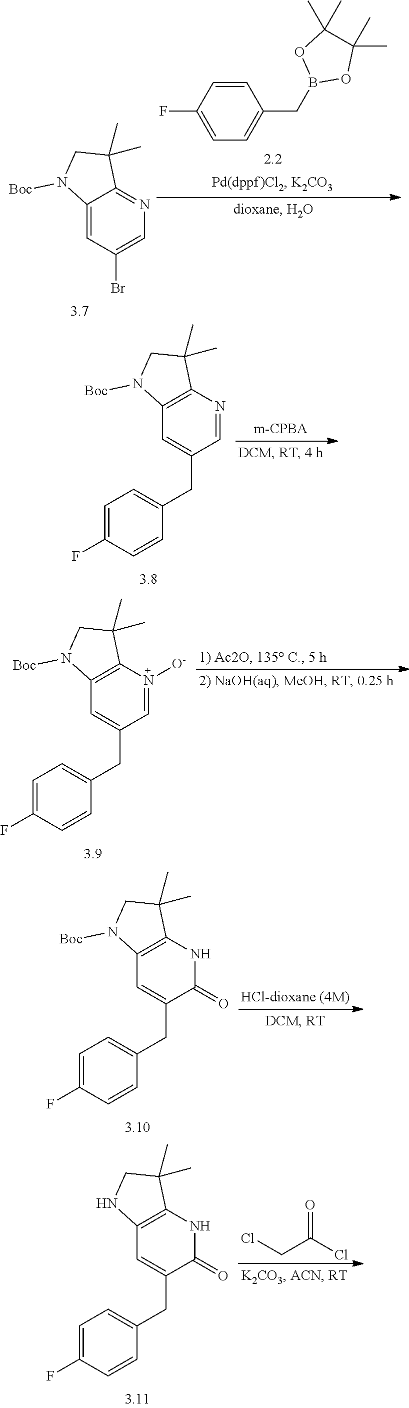

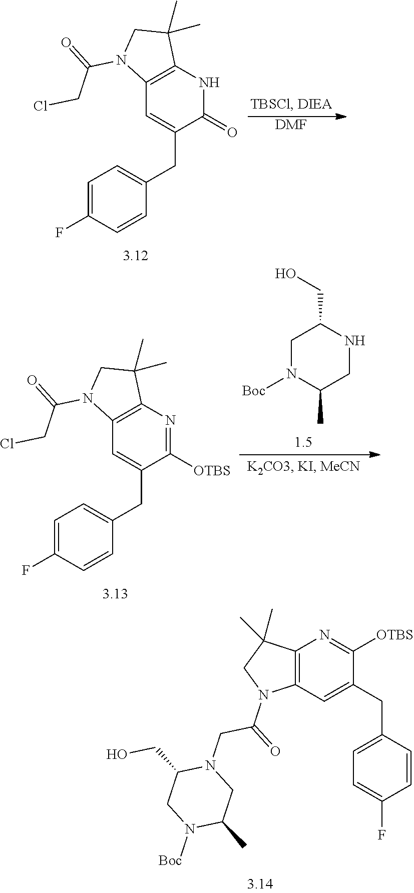

### Step 1 of 13: Synthesis of Intermediate 3.2, dimethyl 2-(5-bromo-3-nitropyridin-2-yl)malonate

Two batches were carried out in parallel: To a suspension of K₂CO₃ (464 g, 3.36 mol, 3.02 eq) in DMF (1000 mL) was added dropwise CH₂(CO₂Me)₂ (220.4 g, 1.67 mol, 192 mL, 1.50 eq) over 20 min at 0° C. followed by portion-wise addition of 5-bromo-2-chloro-3-nitropyridine (264 g, 1.11 mol, 1 eq). The reaction mixture was allowed to warm to 20° C., and stirred for 18 h. TLC (Petroleum ether/Ethyl acetate=2:1) indicated the reaction was almost complete. The two reactions were combined for work-up. The reaction mixture was poured into 2.0 M HCl (6.2 L) and extracted with EtOAc (3 L*3). The combined organic layers were washed with LiCl (1.0 M aqueous solution, 2 L) and brine (2 L), dried over Na₂SO₄, filtered, and concentrated in vacuo to give the crude product. The crude product was purified by column chromatography on silica gel (Petroleum ether/Ethyl acetate=20:1 to 1:1) to give the title compound (832 g, crude) as a yellow oil (containing dimethyl malonate residue).

TLC (Petroleum ether/Ethyl acetate=2:1) R_{f}=0.52.

¹H NMR (400 MHz, CHLOROFORM-d) 6=8.88 (d, J=1.9 Hz, 1H), 8.63 (d, J=1.9 Hz, 1H), 5.49 (s, 1H), 3.82 (s, 6H).

### Step 2 of 13: Synthesis of Intermediate 3.3, methyl 2-(5-bromo-3-nitropyridin-2-yl)acetate

Two reactions were carried out in parallel:

To a suspension of dimethyl 2-(5-bromo-3-nitropyridin-2-yl)malonate (416 g, 1.25 mol, 1 eq) in H₂O (600 mL) was added LiCl (265 g, 6.24 mol, 5 eq). The reaction mixture was heated at reflux for 72 h after which time TLC (Petroleum ether/Ethyl acetate=2:1) showed complete consumption of starting material. The two reactions were combined for work-up. The cooled reaction mixture was partitioned between EtOAc (3000 mL) and water (1000 mL). The isolated organic phase was dried over anhydrous Na₂SO₄, filtered, and concentrated in vacuo to give the crude product as a brown oil. The residue was purified by column chromatography (SiO₂, Petroleum ether/Ethyl acetate=50:1 to 20:1) to give the title compound (540 g, 1.96 mol, 77.1% yield) as a yellow oil.

¹H NMR (400 MHz, CHLOROFORM-d) δ ppm 8.85 (d, J=2.00 Hz, 1H), 8.58 (d, J=2.00 Hz, 1H), 4.29 (s, 2H), 3.73 (s, 3H).

### Step 3 of 13: Synthesis of Intermediate 3.4, methyl 2-(5-bromo-3-nitropyridin-2-yl)-2-methylpropanoate

Four reactions were carried out in parallel:

To a solution of methyl 2-(5-bromo-3-nitropyridin-2-yl)acetate (50.0 g, 182 mmol, 1 eq) in DMF (500 mL) was added NaH (16.0 g, 400 mmol, 60% purity, 2.2 eq) at 0° C. The mixture was stirred at 0° C. for 30 min, then Mel (64.5 g, 454 mmol, 28.3 mL, 2.5 eq) was added dropwise to the mixture at 0° C. over one hour. The mixture was stirred at 20° C. for 0.5 h after which time TLC (Petroleum ether/Ethyl acetate=10:1) indicated complete consumption of starting material and formation of a new product. The four reactions were combined for work-up. The reaction was quenched with saturated aqueous NH₄Cl (3000 mL) at 0° C. and the resulting mixture was extracted with EtOAc (3000 mL*2). The combined organics were washed with brine (1000 mL), dried over anhydrous Na₂SO₄, filtered, and concentrated in vacuo. The residue was purified by column chromatography (SiO₂, Petroleum ether/Ethyl acetate=1:0 to 4:1) to give the title compound (160 g, 528 mmol, 74.4% yield) as a yellow oil.

¹H NMR (400 MHz, CHLOROFORM-d) δ ppm 8.83 (d, J=2.13 Hz, 1H), 8.37 (d, J=2.13 Hz, 1H), 3.65 (s, 3H), 1.68 (s, 6H).

### Step 4 of 13: Synthesis of Intermediate 3.5, 6-bromo-3,3-dimethyl-1,3-dihydro-2H-pyrrolo[3,2-b]pyridin-2-one

Two reactions were carried out in parallel:

To a solution of methyl 2-(5-bromo-3-nitropyridin-2-yl)-2-methylpropanoate (80.0 g, 264 mmol, 1 eq) in AcOH (1000 mL) was added iron powder (73.7 g, 1.32 mol, 5 eq). The mixture was stirred at 100° C. for 1.5 h after which time TLC (Petroleum ether/Ethyl acetate=2:1) indicated complete consumption of starting material and formation of one new spot. The two reactions were combined for work-up. The mixture was filtered and the filtrate was concentrated to dryness. The crude product was treated with saturated aqueous NaHCO₃ (2000 mL) and then extracted with EtOAc (1500 mL*3). The combined organic phases were washed with brine (500 mL), dried over Na₂SO₄, filtered, and concentrated under reduced pressure to give the title compound (120 g, 498 mmol, 94.3% yield) as a yellow solid.

¹H NMR (400 MHz, CHLOROFORM-d) δ ppm 1.45 (s, 6H) 7.41 (d, J=1.75 Hz, 1H) 8.28 (d, J=1.75 Hz, 1H) 9.94 (br s, 1H).

### Step 5 of 13: Synthesis of Intermediate 3.6, 6-bromo-3,3-dimethyl-2,3-dihydro-1H-pyrrolo[3,2-b]pyridine

Three reactions were carried out in parallel:

To a solution of 6-bromo-3,3-dimethyl-1,3-dihydro-2H-pyrrolo[3,2-b]pyridin-2-one (45 g, 187 mmol, 1 eq) in THE (900 mL) was added NaBH₄ (35.3 g, 933 mmol, 5 eq) and BF₃·Et₂O (185 g, 1.30 mol, 161 mL, 7 eq) at 0° C. under N₂. The mixture was stirred at 20° C. for 12 h after which time TLC (Petroleum ether/Ethyl acetate=2:1) indicated complete consumption of starting material and formation of a new product. The three reactions were quenched separately with saturated aqueous NH₄Cl (300 mL) and extracted with EtOAc (1000 mL*2). The combined organic phases were washed with brine (500 mL), dried over anhydrous Na₂SO₄, filtered, and concentrated in vacuo. The three residues were combined and purified by column chromatography (SiO₂, Petroleum ether/Ethyl acetate=10:1 to 5:1) to give the title compound (90.0 g, 396 mmol, 71.0% yield) as a white solid.

1H NMR (400 MHz, CHLOROFORM-d) δ ppm 1.32 (s, 6H) 3.40 (s, 2H) 6.89 (d, J=1.75 Hz, 1H) 7.88 (d, J=1.75 Hz, 1H).

### Step 6 of 13: Synthesis of Intermediate 3.7, tert-butyl 6-bromo-3,3-dimethyl-2,3-dihydro-1H-pyrrolo[3,2-b]pyridine-1-carboxylate

Two reactions were carried out in parallel:

To a solution of 6-bromo-3,3-dimethyl-2,3-dihydro-1H-pyrrolo[3,2-b]pyridine (55.0 g, 242 mmol, 1 eq) and Boc₂O (38.7 g, 315 mmol, 1.3 eq) in DCM (900 mL) was added DMAP (32.5 g, 266 mmol, 1.1 eq) in portions at 0° C. The mixture was stirred at 30° C. for 12 h after which time TLC (Petroleum ether/Ethyl acetate=2:1) indicated complete consumption of the starting material. The two reactions were combined for work-up. The mixture was filtered and the filtrate was concentrated to give a crude product. The residue was purified by column chromatography (SiO₂, Petroleum ether/Ethyl acetate=1:0 to 4:1) to give the title compound (101 g, 309 mmol, 67.8% yield) as a colorless oil.

¹H NMR (400 MHz, CHLOROFORM-d) δ ppm 8.15 (d, J=2.00 Hz, 1H), 3.74 (br s, 2H), 1.49-1.63 (m, 9H), 1.36 (s, 6H).

### Step 7 of 13: Synthesis of Intermediate 3.8, tert-butyl 6-(4-fluorobenzyl)-3,3-dimethyl-2,3-dihydro-1H-pyrrolo[3,2-b]pyridine-1-carboxylate

Two reactions were carried out in parallel:

To a solution of tert-butyl 6-bromo-3,3-dimethyl-2,3-dihydro-1H-pyrrolo[3,2-b]pyridine-1-carboxylate (50 g, 153 mmol, 1 eq), and 2-(4-fluorobenzyl)-4,4,5,5-tetramethyl-1,3,2-dioxaborolane (50.5 g, 214 mmol, 1.4 eq) in dioxane (800 mL) and H₂O (80 mL) was added K₂CO₃ (52.8 g, 382 mmol, 2.5 eq) and Pd(dppf)Cl₂ (16.8 g, 22.9 mmol, 0.15 eq). The mixture was stirred at 80° C. for 12 h under N₂ after which time TLC (Petroleum ether/Ethyl acetate=3:1) indicated conversion to a new product. The two reactions were combined for work-up. The mixture was filtered and the filtrate was concentrated to dryness. The residue was purified by column chromatography (SiO₂, Petroleum ether/Ethyl acetate=40:1 to 5:1) to give the title compound (64.0 g, 180 mmol, 58.8% yield) as a yellow oil.

LCMS (ES, m/z): 357.3 [M+H]⁺.

### Step 8 of 13: Synthesis of Intermediate 3.9, 1-(tert-butoxycarbonyl)-6-(4-fluorobenzyl)-3,3-dimethyl-2,3-dihydro-1H-pyrrolo[3,2-b]pyridine 4-oxide

To a solution of tert-butyl 6-(4-fluorobenzyl)-3,3-dimethyl-2,3-dihydro-1H-pyrrolo[3,2-b]pyridine-1-carboxylate (35.0 g, 98.2 mmol 1 eq) in DCM (600 mL) was added m-CPBA (25.9 g, 128 mmol, 85% purity, 1.3 eq) at 0° C. The mixture was stirred at 15° C. for 4 h after which time LCMS indicated formation of a product of desired mass. The reaction was quenched with saturated aqueous Na₂SO₃ (600 mL) and then extracted with DCM (100 mL*2). The combined organics were washed with 1N NaOH (300 mL*2) and brine (300 mL), dried over anhydrous Na₂SO₄, filtered, and concentrated in vacuo to give the title compound (34 g, 91.3 mmol, 95.7% yield) as a red oil.

LCMS (ES, m/z): 373.3 [M+H]⁺.

### Step 9 of 13: Synthesis of Intermediate 3.10, tert-butyl 6-(4-fluorobenzyl)-3,3-dimethyl-5-oxo-2,3,4,5-tetrahydro-1H-pyrrolo[3,2-b]pyridine-1-carboxylate

A mixture of 1-(tert-butoxycarbonyl)-6-(4-fluorobenzyl)-3,3-dimethyl-2,3-dihydro-1H-pyrrolo[3,2-b]pyridine 4-oxide (34.0 g, 91.3 mmol, 1 eq) in Ac₂O (340 g, 315 mL) was stirred at 135° C. for 6 h after which time LCMS indicated complete consumption of starting material. The reaction was cooled to ambient temperature and the resulting solution was poured into ice-water (2000 g). The resulting brown solid was collected by filtration, dissolved in MeOH (300 mL), and treated with NaOH (1 M, 408 mL) with stirring at 15° C. for 1 h. LCMS indicated that the intermediate product was consumed and a new product of desired mass was detected. The mixture was concentrated to remove most of the MeOH under reduced pressure and treated with H₂O (1500 mL) with stirring at 15° C. for 1 h. The solids were filtered off and the filter cake was dried to give the title compound (32.0 g, 85.0 mmol, 94.1% yield) as a white solid.

LCMS (ES, m/z): 373.3 [M+H]⁺.

### Step 10 of 13: Synthesis of Intermediate 3.11, 6-(4-fluorobenzyl)-3,3-dimethyl-1,2,3,4-tetrahydro-5H-pyrrolo[3,2-b]pyridin-5-one

Two reactions were carried out in parallel:

To a solution of tert-butyl 6-(4-fluorobenzyl)-3,3-dimethyl-5-oxo-2,3,4,5-tetrahydro-1H-pyrrolo[3,2-b]pyridine-1-carboxylate (16 g, 43.0 mmol, 1 eq) in DCM (270 mL) was added HCl/dioxane (4 M, 270 mL, 25.1 eq) and the mixture was stirred for 12 h at 15° C. after which time LCMS indicated complete consumption of starting material and formation of a product of target mass. The two reactions were combined for work-up. The mixture was filtered and the filter cake was dried to give the title compound (26 g, 84.2 mmol, 100% yield, HCl) as a yellow solid.

LCMS (ES, m/z): 273.1 [M+H]⁺.

### Step 11 of 13: Synthesis of Intermediate 3.12, 1-(2-chloroacetyl)-6-(4-fluorobenzyl)-3,3-dimethyl-1,2,3,4-tetrahydro-5H-pyrrolo[3,2-b]pyridin-5-one

To a mixture of 6-(4-fluorobenzyl)-3,3-dimethyl-1,2,3,4-tetrahydro-5H-pyrrolo[3,2-b]pyridin-5-one (26.0 g, 84.2 mmol, 1 eq, HCl) and K₂CO₃ (29.1 g, 210 mmol, 2.5 eq) in CH₃CN (500 mL) was added a solution of 2-chloroacetyl chloride (11.9 g, 105 mmol, 8.40 mL, 1.3 eq) dissolved in CH₃CN (40 mL) dropwise with stirring at 0° C. The mixture was warmed to 15° C. and stirred for 4 h after which time LCMS indicated complete consumption of starting material and formation of a product of target mass. MeOH (200 mL) was added and the mixture was stirred at 20° C. for 0.5 h. The mixture was concentrated to dryness and the residue was treated with H₂O (500 mL) and extracted with EtOAc (400 mL*3). The combined organic phases were washed with brine (200 mL), dried over Na₂SO₄, filtered, and concentrated under reduced pressure to give a residue. The residue was purified by column chromatography (SiO₂, Petroleum ether/Ethyl acetate=10/1 to 0/1) to give the title compound (23.0 g, 65.9 mmol, 72.4% yield) as a yellow solid.

LCMS (ES, m/z): 349.2 [M+H]⁺.

### Step 12 of 13: Synthesis of Intermediate 3.13, 1-(5-((tert-butyldimethylsilyl)oxy)-6-(4-fluorobenzyl)-3,3-dimethyl-2,3-dihydro-1H-pyrrolo[3,2-b]pyridin-1-yl)-2-chloroethan-1-one

To a solution of 1-(2-chloroacetyl)-6-(4-fluorobenzyl)-3,3-dimethyl-1,2,3,4-tetrahydro-5H-pyrrolo[3,2-b]pyridin-5-one (20.0 g, 57.3 mmol, 1 eq) and DIEA (18.5 g, 143 mmol, 25 mL, 2.5 eq) in DMF (300 mL) was added TBDMSCl (9.51 g, 63.1 mmol, 1.1 eq) at 0° C. The mixture was stirred at 15° C. for 12 h after which time LCMS indicated complete consumption of starting material and formation of a new product of target mass. The reaction mixture was poured into saturated aqueous NH₄Cl (200 mL) and extracted with EtOAc (200 mL*3). The combined organic phases were washed with brine (200 mL), dried over Na₂SO₄, filtered, and concentrated under reduced pressure to give a residue. The residue was purified by column chromatography (SiO₂, Petroleum ether/Ethyl acetate=1:0 to 4:1) to give the title compound (20.2 g, 43.6 mmol, 76.1% yield) as a white solid. LCMS (ES, m/z): 463.2 [M+H]⁺.

### Step 13 of 13: Synthesis of Intermediate 3.14, tert-butyl (2R,5R)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-5-(hydroxymethyl)-2-methylpiperazine-1-carboxylate

A mixture of tert-butyl (2R,5R)-5-(hydroxymethyl)-2-methyl-piperazine-1-carboxylate (14.9 g, 64.8 mmol, 1.5 eq), 1-(5-((tert-butyldimethylsilyl)oxy)-6-(4-fluorobenzyl)-3,3-dimethyl-2,3-dihydro-1H-pyrrolo[3,2-b]pyridin-1-yl)-2-chloroethan-1-one (20 g, 43.2 mmol, 1 eq), K₂CO₃ (11.9 g, 86.4 mmol, 2 eq), and KI (7.17 g, 43.2 mmol, 1 eq) in MeCN (400 mL) was stirred at 50° C. for 12 h after which time TLC (Petroleum ether/Ethyl acetate=3:1) indicated nearly complete conversion of starting material to a new product. The mixture was filtered and the filtrate was concentrated to dryness. The residue was purified by column chromatography (SiO₂, Petroleum ether/Ethyl acetate=1:0 to 0:1) to give the title compound (26.5 g, 40.3 mmol, 93.4% yield) as a yellow solid.

LCMS (ES, m/z): 657.5 [M+H]⁺.

### Preparation of Intermediate 4.4, tert-butyl (2R,5R)-4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-(hydroxymethyl)-2-methylpiperazine-1-carboxylate

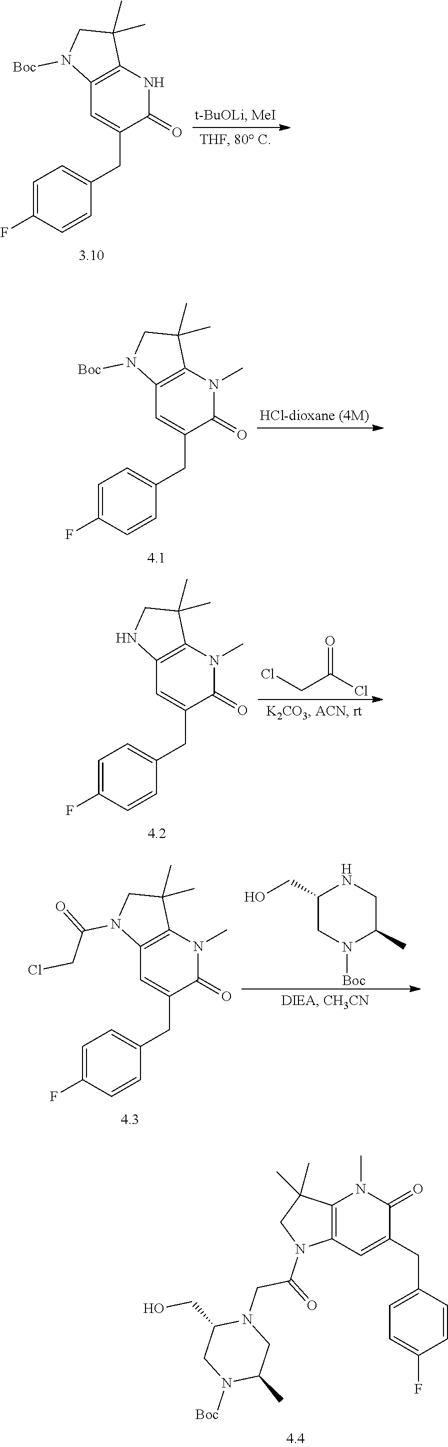

### Step 1 of 4: Synthesis of Intermediate 4.1, tert-butyl 6-(4-fluorobenzyl)-3,3,4-trimethyl-5-oxo-2,3,4,5-tetrahydro-1H-pyrrolo[3,2-b]pyridine-1-carboxylate

To a solution of tert-butyl 6-(4-fluorobenzyl)-3,3-dimethyl-5-oxo-2,3,4,5-tetrahydro-1H-pyrrolo[3,2-b]pyridine-1-carboxylate (4 g, 10.7 mmol, 1 eq) in THF (50 mL) was added t-BuOLi (1.72 g, 21.5 mmol, 1.94 mL, 2 eq) and Mel (3.05 g, 21.5 mmol, 1.34 mL, 2 eq) and the reaction mixture was stirred at 80° C. for 12 h. LC-MS indicated complete consumption of starting material with formation of a product of desired mass. The mixture was concentrated in vacuo and the residue was purified by column chromatography (SiO₂, Petroleum ether/Ethyl acetate=10/1 to 0/1) to give the title compound (3.20 g, 8.28 mmol, 77.1% yield) as a yellow solid.

LCMS (ES, m/z): 387.1 [M+H]⁺.

¹H NMR (400 MHz, CHLOROFORM-d) δ=8.13 (br s, 1H), 7.22 (br s, 1H), 6.95 (br s, 2H), 3.83 (s, 2H), 3.71-3.56 (m, 5H), 1.56-1.30 (m, 15H).

### Step 2 of 4: Synthesis of Intermediate 4.2, 6-(4-fluorobenzyl)-3,3,4-trimethyl-1,2,3,4-tetrahydro-5H-pyrrolo[3,2-b]pyridin-5-one

To a solution of tert-butyl 6-(4-fluorobenzyl)-3,3,4-trimethyl-5-oxo-2,3,4,5-tetrahydro-1H-pyrrolo[3,2-b]pyridine-1-carboxylate (3.20 g, 8.28 mmol, 1 eq) in DCM (30 mL) was added HCl/dioxane (4 M, 20 mL, 9.7 eq). The mixture was stirred at 25° C. for 12 h after which time LC-MS indicated complete consumption of starting material and formation of a product of target mass. The mixture was concentrated in vacuo to give the title compound (2.67 g, crude, HCl) as a yellow oil.

LCMS (ES, m/z): 287.1 [M+H]⁺.

### Step 3 of 4: Synthesis of Intermediate 4.3, 1-(2-chloroacetyl)-6-(4-fluorobenzyl)-3,3,4-trimethyl-1,2,3,4-tetrahydro-5H-pyrrolo[3,2-b]pyridin-5-one

A mixture of 6-(4-fluorobenzyl)-3,3,4-trimethyl-1,2,3,4-tetrahydro-5H-pyrrolo[3,2-b]pyridin-5-one (2.67 g, 9.32 mmol, 1 eq, HCl) and K₂CO₃ (2.84 g, 20.5 mmol, 2.2 eq) in MeCN (20 mL) was treated with a solution of 2-chloroacetyl chloride (1.26 g, 11.19 mmol, 890 μL, 1.2 eq) in MeCN (5 mL) dropwise with stirring at 0° C. The mixture was allowed to warm to 25° C. and stirred for 2 h after which time LC-MS showed consumption of starting material and formation of a product of target mass. The reaction was quenched with MeOH (10 mL) and the mixture was concentrated in vacuo. The residue was extracted with EtOAc (10 mL*2) and the combined organic phases were washed with brine (20 mL), dried over anhydrous Na₂SO₄, filtered, and concentrated in vacuo. The residue was purified by column chromatography (SiO₂, Petroleum ether/Ethyl acetate=10/1 to 0/1) to give the title compound (2.7 g, 7.44 mmol, 79.8% yield) as a white solid. TLC (Petroleum ether/Ethyl acetate=0:1) R_{f}=0.43.

LCMS (ES, m/z): 363.1 [M+H]⁺.

¹H NMR (400 MHz, CHLOROFORM-d) δ=8.13 (br s, 1H), 7.22 (br s, 1H), 6.95 (br s, 2H), 3.83 (s, 2H), 3.71-3.56 (m, 5H), 1.56-1.30 (m, 15H).

### Step 4 of 4: Synthesis of Intermediate 4.4, tert-butyl (2R,5R)-4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-(hydroxymethyl)-2-methylpiperazine-1-carboxylate

To a solution of 1-(2-chloroacetyl)-6-(4-fluorobenzyl)-3,3,4-trimethyl-1,2,3,4-tetrahydro-5H-pyrrolo[3,2-b]pyridin-5-one (2.70 g, 7.44 mmol, 1 eq) and tert-butyl (2R,5R)-5-(hydroxymethyl)-2-methylpiperazine-1-carboxylate (2.23 g, 9.67 mmol, 1.3 eq) in THE (30 mL) was added DIEA (2.89 g, 22.3 mmol, 3.89 mL, 3 eq). The mixture was stirred at 80° C. for 12 h after which time LC-MS indicated consumption of starting material and formation of a new product of target mass. The mixture was concentrated in vacuo and the residue was purified by column chromatography (SiO₂, Petroleum ether/Ethyl acetate=5/1 to 0/1) to give the title compound (3.5 g, 6.29 mmol, 58.3% yield) as a yellow solid.

LCMS (ES, m/z): 557.3 [M+H]⁺.

TLC (Petroleum ether/Ethyl acetate=0:1) R_{f}=0.10.

¹H NMR (400 MHz, CHLOROFORM-d) δ=8.34 (s, 1H), 7.32-7.28 (m, 2H), 6.96 (t, J=8.7 Hz, 2H), 4.19-4.09 (m, 2H), 3.97-3.87 (m, 2H), 3.85-3.74 (m, 5H), 3.64-3.55 (m, 5H), 3.54-3.42 (m, 2H), 3.29 (dd, J=4.1, 14.1 Hz, 1H), 2.98-2.89 (m, 2H), 2.63 (br dd, J=5.1, 12.1 Hz, 1H), 1.47 (s, 12H), 1.23 (d, J=6.5 Hz, 3H).

### Preparation of Intermediate 5.3, tert-butyl (2R,5R)-4-(2-(6-(4-fluorobenzyl)-3,3-dimethyl-2,3-dihydro-1H-pyrrolo[3,2-b]pyridin-1-yl)-2-oxoethyl)-5-(hydroxymethyl)-2-methylpiperazine-1-carboxylate

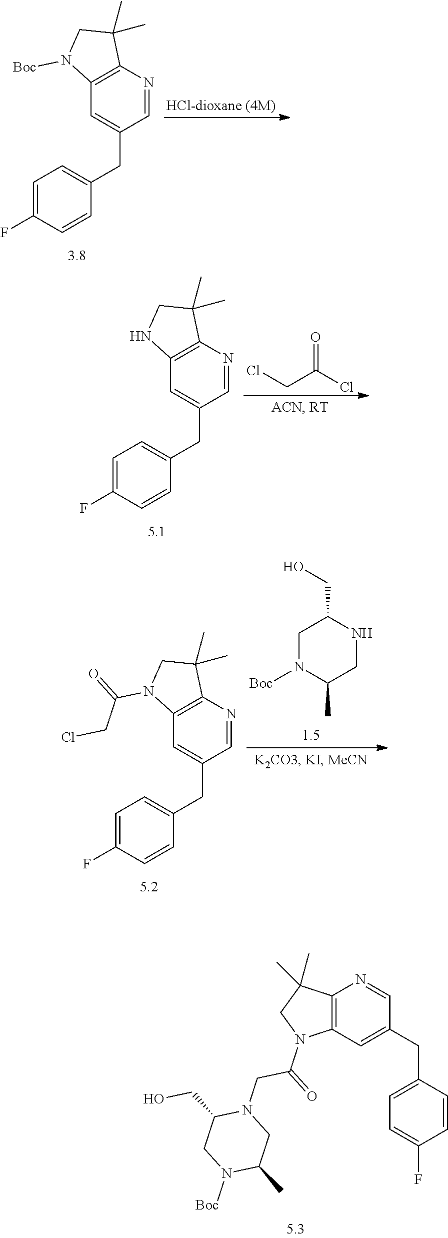

### Step 1 of 3: Synthesis of Intermediate 5.1, 6-(4-fluorobenzyl)-3,3-dimethyl-2,3-dihydro-1H-pyrrolo[3,2-b]pyridine

A solution of 6-(4-fluoro-benzyl)-3,3-dimethyl-2,3-dihydro-pyrrolo[3,2-b]pyridine-1-carboxylic acid tert-butyl ester (1.50 g, 4.21 mmol) in dichloromethane (10 mL) was treated with HCl (4 M in dioxane, 10 mL) and the mixture was stirred at room temperature overnight. The solvent was evaporated and the residue was partitioned between saturated aqueous sodium bicarbonate (200 mL) and ethyl acetate (3×60 mL). The combined organics were dried over Na₂SO₄, filtered, and evaporated in vacuo to afford the title compound (0.72 g, 2.81 mmol, 94% yield) as a light brown solid.

LCMS (ES, m/z): 257.1 [M+H]⁺.

¹H NMR (400 MHz, CDCl₃): δ ppm 7.78 (1H, s), 7.26 (2H, dd), 6.99-6.94 (2H, t), 6.57 (1H, d), 3.81 (2H, s), 3.36 (2H, s), 1.34 (6H, s).

### Step 2 of 3: Synthesis of Intermediate 5.2, 2-chloro-1-(6-(4-fluorobenzyl)-3,3-dimethyl-2,3-dihydro-1H-pyrrolo[3,2-b]pyridin-1-yl)ethan-1-one

To a stirred suspension of 6-(4-fluoro-benzyl)-3,3-dimethyl-2,3-dihydro-1H-pyrrolo[3,2-b]pyridine (500 mg, 1.95 mmol, HCl) in acetonitrile (10 mL) at 20° C. was added, steadily over 2 min, a solution of chloroacetyl chloride (287 mg, 2.53 mmol) in acetonitrile (1 mL) while maintaining the reaction temperature at or below 20° C. using an external ice-methanol bath. The resulting mixture was stirred at room temperature for 1.5 hours, concentrated, and the residue was diluted with methanol. The solution was stirred at room temperature for 5 min. followed by addition of 10% potassium carbonate solution. The mixture was extracted with ethyl acetate three times. The ethyl acetate layers were combined, washed with brine, dried over sodium sulphate, and concentrated to dryness. The residue was purified by column chromatography on silica gel eluting with petroleum/ethyl acetate (3:1) to afford the title compound (580 mg, 1.74 mmol, 89.2% yield), as a white solid.

LCMS (ES, m/z): 332.8 [M+H]⁺.

### Step 3 of 3, Synthesis of Intermediate 5.3, tert-butyl (2R,5R)-4-(2-(6-(4-fluorobenzyl)-3,3-dimethyl-2,3-dihydro-1H-pyrrolo[3,2-b]pyridin-1-yl)-2-oxoethyl)-5-(hydroxymethyl)-2-methylpiperazine-1-carboxylate

Finely ground potassium iodide (578 mg, 3.48 mmol) was added to a mixture of (2R,5R)-5-hydroxymethyl-2-methyl-piperazine-1-carboxylic acid tert-butyl ester (481 mg, 2.09 mmol), 2-chloro-1-[6-(4-fluorobenzyl)-3,3-dimethyl-2,3-dihydro-pyrrolo[3,2-b]pyridin-1-yl]-ethanone hydrochloride (580 mg, 1.74 mmol), potassium carbonate (960 mg, 6.96 mmol) and acetonitrile (30 mL) under nitrogen. The mixture was stirred at 20° C. overnight. The reaction mixture was concentrated under reduced pressure and the residue was partitioned between water and ethyl acetate. The organic phase was dried over Na₂SO₄, filtered, and evaporated in vacuo to give the title compound (760 mg, 1.44 mmol, 82.7% yield).

LCMS (ES, m/z): 526.8 [M+H]⁺.

### Preparation of Intermediate 6.2, tert-butyl (2R,5S)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methyl-5-(((R)-2-methylpiperazin-1-yl)methyl)piperazine-1-carboxylate

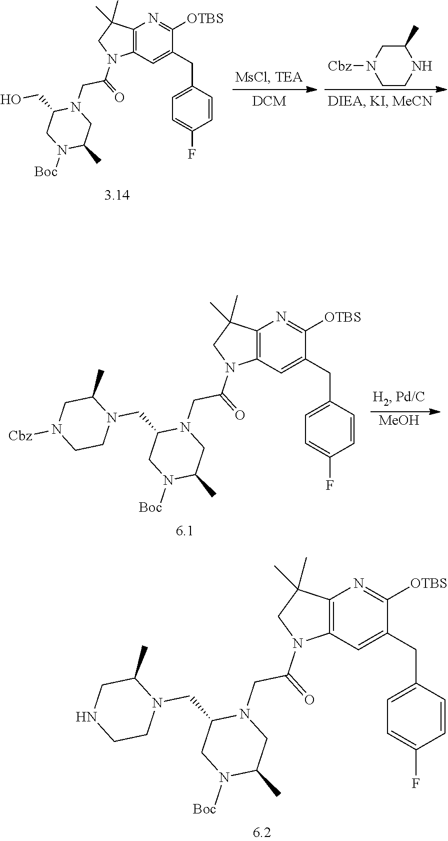

### Step 1 of 2: Synthesis of Intermediate 6.1, tert-butyl (2R,5S)-5-(((R)-4-((benzyloxy)carbonyl)-2-methylpiperazin-1-yl)methyl)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate

To a solution of tert-butyl (2R,5R)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-5-(hydroxymethyl)-2-methylpiperazine-1-carboxylate (10.0 g, 15.2 mmol, 1 eq) and DIEA (4.90 g, 38.1 mmol, 6.60 mL, 2.5 eq) in DCM (120 mL) was added MsCl (2.09 g, 18.3 mmol, 1.4 mL, 1.2 eq) dropwise at 0° C. The mixture was stirred at 15° C. for 1 h after which time TLC (Petroleum ether/Ethyl acetate=0:1) indicated complete conversion of starting material to a new product. Water (100 mL) was added to the mixture and extracted with DCM (50 mL*2). The combined organic phases were washed with brine (50 mL), dried over Na₂SO₄, filtered, and concentrated under reduced pressure to give the intermediate product (11.2 g, crude) as a yellow oil.

A mixture of the intermediate product (11.2 g, 15.2 mmol, 1 eq), benzyl (R)-3-methylpiperazine-1-carboxylate (3.93 g, 16.8 mmol, 1.1 eq), KI (2.53 g, 15.2 mmol, 1 eq), and DIEA (5.91 g, 45.7 mmol, 7.96 mL, 3 eq) in MeCN (150 mL) was stirred at 80° C. for 12 h. TLC indicated complete consumption of starting material with formation of a new product. The mixture was filtered and the filtrate was concentrated to dryness. The residue was purified by column chromatography (SiO₂, Petroleum ether/Ethyl acetate=1:0 to 2:1) to give the title compound (10.8 g, 12.3 mmol, 81.2% yield) as a yellow oil.

LCMS (ES, m/z): 873.8 [M+H]⁺.

### Step 2 of 2: Synthesis of Intermediate 6.2, tert-butyl (2R,5S)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methyl-5-(((R)-2-methylpiperazin-1-yl)methyl)piperazine-1-carboxylate

To a solution of tert-butyl (2R,5S)-5-(((R)-4-((benzyloxy)carbonyl)-2-methylpiperazin-1-yl)methyl)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate (5.00 g, 5.73 mmol, 1 eq) in MeOH (250 mL) was added Pd/C (4.00 g, 10% purity) under N₂ atmosphere. The suspension was degassed and purged with H₂. The mixture was stirred under H₂ (15 psi) at 50° C. for 12 h after which time LCMS showed complete consumption of starting material and formation of a product of target mass. The mixture was filtered and the filtrate was concentrated to give crude title compound (3.5 g, 4.74 mmol, quantitative yield) as a yellow solid.

LCMS (ES, m/z): 739.5 [M+H]⁺. Minor peak of 625.4 [M+H]⁺ corresponding to TBS cleaved product also noted.

### Preparation of Intermediate 7.2, tert-butyl (2R,5S)-4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-2-methyl-5-(((R)-2-methylpiperazin-1-yl)methyl)piperazine-1-carboxylate

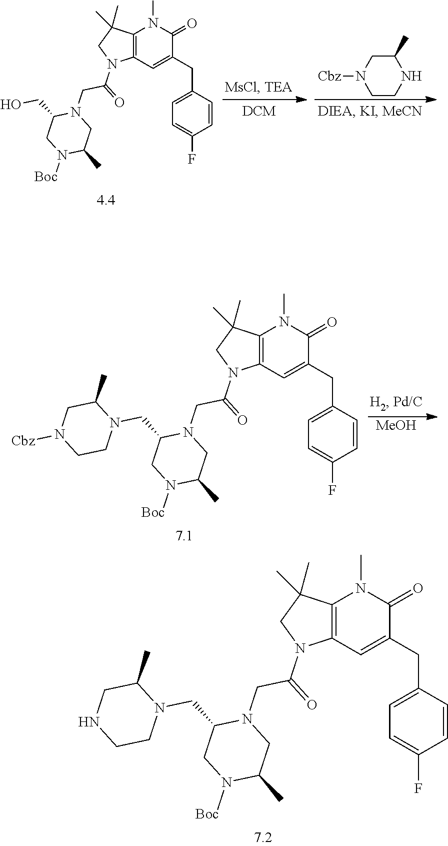

### Step 1 of 2: Synthesis of Intermediate 7.1, tert-butyl (2R,5S)-5-(((R)-4-((benzyloxy)carbonyl)-2-methylpiperazin-1-yl)methyl)-4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-2-methylpiperazine-1-carboxylate

To a solution of tert-butyl (2R,5R)-4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-(hydroxymethyl)-2-methylpiperazine-1-carboxylate (500 mg, 898 umol, 1 eq) and TEA (136 mg, 1.35 mmol, 187 uL, 1.5 eq) in DCM (5 mL) was added MsCl (123 mg, 1.08 mmol, 83.4 uL, 1.2 eq) at 0° C. The mixture was stirred at 25° C. for 0.5 h after which time TLC indicated conversion of starting material to a new product. The reaction was quenched with saturated aqueous NaHCO₃ (20 mL) and then extracted with DCM (10 mL*2). The combined organic phases were washed with brine (10 mL), dried over anhydrous Na₂SO₄, filtered, and concentrated in vacuo to give the intermediate product (500 mg, crude) as a yellow solid. To a solution of the intermediate product (1.00 g, 1.74 mmol, 1 eq), and benzyl (R)-3-methylpiperazine-1-carboxylate (367 mg, 1.56 mmol, 0.9 eq) in MeCN (10 mL) was added DIEA (674 mg, 5.22 mmol, 909 μL, 3 eq) and KI (289 mg, 1.74 mmol, 1 eq). The mixture was stirred at 80° C. for 12 h after which time TLC (Dichloromethane/Methanol=15:1, R_{f}=0.23) indicated complete consumption of starting material and formation of a major new product. The reaction mixture was filtered and the filtrate was concentrated in vacuo. The residue was dissolved in EtOAc and filtered. The filtrate was concentrated in vacuo to give the title compound (1.3 g, crude) as a yellow oil.

LCMS (ES, m/z): 773.5 [M+H]⁺.

### Step 2 of 2: Synthesis of Intermediate 7.2, tert-butyl (2R,5S)-4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-2-methyl-5-(((R)-2-methylpiperazin-1-yl)methyl)piperazine-1-carboxylate

To a solution of tert-butyl (2R,5S)-5-(((R)-4-((benzyloxy)carbonyl)-2-methylpiperazin-1-yl)methyl)-4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-2-methylpiperazine-1-carboxylate (1 g, 1.29 mmol, 1 eq) in MeOH (5 mL) was added Pd/C (50 mg, 129 umol, 10% purity) under N₂. The suspension was degassed under vacuum and purged with H₂ three times. The mixture was stirred under H₂ (15 psi) at 25° C. for 12 h after which time LCMS complete consumption of starting material and formation of one main peak of desired mass. The reaction mixture was filtered and the filtrate was concentrated in vacuo to give the title compound (800 mg, crude) as a yellow solid.

LCMS (ES, m/z): 639.4 [M+H]⁺.

### Preparation of Intermediate 8.2, tert-butyl (2R,5S)-5-(((S)-3-aminopiperidin-1-yl)methyl)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate

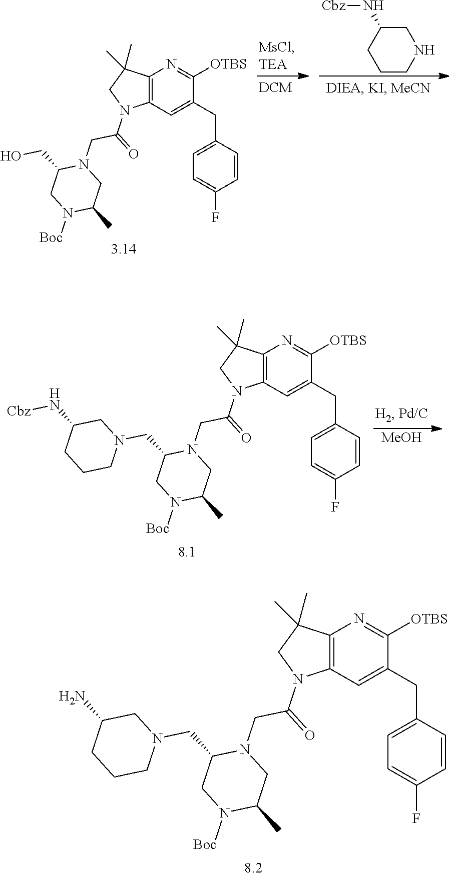

### Step 1 of 2: Synthesis of Intermediate 8.1, tert-butyl (2R,5S)-5-(((S)-3-(((benzyloxy)carbonyl)amino)piperidin-1-yl)methyl)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate

MsCl (290 mg, 2.53 mmol) was added to a solution of (2R,5R)-4-{2-[6-(4-fluorobenzyl)-3,3-dimethyl-2,3-dihydro-pyrrolo[3,2-b]pyridin-1-yl]-2-oxo-ethyl}-5-hydroxymethyl-2-methyl-piperazine-1-carboxylic acid tert-butyl ester (760 mg, 1.45 mmol) and triethylamine (293 mg, 2.89 mmol) in dichloromethane (20 mL) at 0° C. The solution was allowed to warm to room temperature and stirred under a nitrogen atmosphere overnight. The mixture was partitioned between aqueous ammonium chloride and dichloromethane and the isolated organic phase was dried over sodium sulphate, filtered, and evaporated in vacuo. The residue was purified by column chromatography on silica gel eluting with petroleum ether/ethyl acetate (1:1) to give the intermediate product (500 mg, 0.917 mmol, 63.7% yield), as a white solid.

A solution of the intermediate (2.24 g, 3.05 mmol, 1 eq), benzyl (S)-piperidin-3-ylcarbamate (785 mg, 3.35 mmol, 1.1 eq), DIEA (1.18 g, 9.14 mmol, 1.59 mL, 3 eq), and KI (506 mg, 3.05 mmol, 1 eq) in CH₃CN (50 mL) was stirred at 80° C. for 12 h after which time LCMS showed formation of product of target mass. The reaction mixture was filtered and concentrated under reduced pressure to give a residue. The residue was purified by column chromatography (SiO₂, Petroleum ether/Ethyl acetate=1:0 to 1:3) to give the title compound (1.40 g, 1.60 mmol, 52.6% yield) as a yellow oil.

TLC (Petroleum ether/Ethyl acetate=1:1) R_{f}=0.43 LCMS (ES, m/z): 873.6 [M+H]⁺.

### Step 2 of 2: Synthesis of Intermediate 8.2, tert-butyl (2R,5S)-5-(((S)-3-aminopiperidin-1-yl)methyl)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate

To a solution of tert-butyl (2R,5S)-5-(((S)-3-(((benzyloxy)carbonyl)amino)piperidin-1-yl)methyl)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate (950 mg, 1.09 mmol, 1 eq) in MeOH (50 mL) was added Pd/C (15 mg, 10% purity) under N₂. The suspension was degassed under vacuum and purged with H₂ several times. The mixture was stirred under H₂ (15 psi) at 50° C. for 12 h after which time LCMS indicated formation of a product of target mass. The mixture was filtered and the filtrate was concentrated to give crude title compound (0.64 g, 866 umol, 79% yield) as a yellow solid.

LCMS (ES, m/z): 739.5 [M+H]⁺. Minor peak of 625.4 [M+H]⁺ corresponding to TBS cleaved product also noted.

Preparation of Intermediate 9.2, tert-butyl (2R,5S)-5-(((R)-3-aminopiperidin-1-yl)methyl)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate.

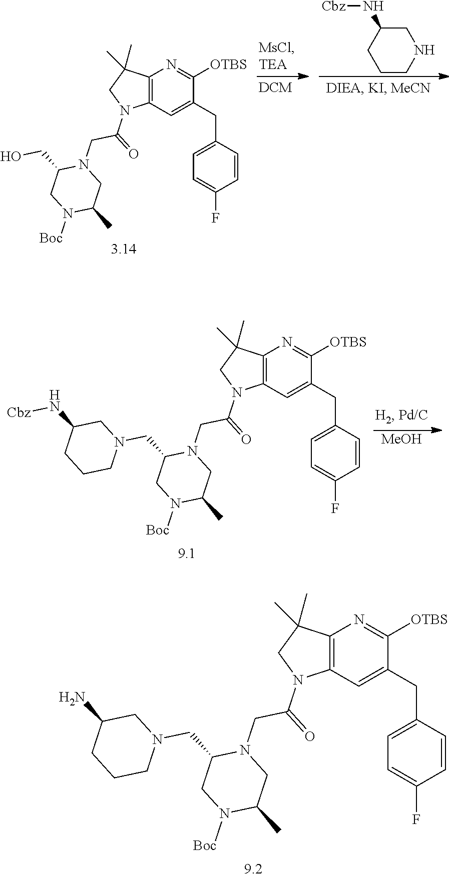

### Step 1 of 2: Synthesis of Intermediate 9.1, tert-butyl (2R,5S)-5-(((R)-3-(((benzyloxy)carbonyl)amino)piperidin-1-yl)methyl)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate

MsCl (290 mg, 2.53 mmol) was added to a solution of (2R,5R)-4-{2-[6-(4-fluorobenzyl)-3,3-dimethyl-2,3-dihydro-pyrrolo[3,2-b]pyridin-1-yl]-2-oxo-ethyl}-5-hydroxymethyl-2-methyl-piperazine-1-carboxylic acid tert-butyl ester (760 mg, 1.45 mmol) and triethylamine (293 mg, 2.89 mmol) in dichloromethane (20 mL) at 0° C. The solution was allowed to warm to room temperature and stirred under a nitrogen atmosphere overnight. The mixture was partitioned between aqueous ammonium chloride and dichloromethane and the isolated organic phase was dried over sodium sulphate, filtered, and evaporated in vacuo. The residue was purified by column chromatography on silica gel eluting with petroleum ether/ethyl acetate (1:1) to give the intermediate product (500 mg, 0.917 mmol, 63.7% yield) as a white solid.

A mixture of the intermediate product (2.8 g, 3.81 mmol, 1 eq), benzyl (R)-piperidin-3-ylcarbamate (982 mg, 4.19 mmol, 1.1 eq), KI (632 mg, 3.81 mmol, 1 eq), and DIEA (1.48 g, 11.4 mmol, 1.99 mL, 3 eq) in MeCN (50 mL) was stirred at 80° C. for 12 h after which time TLC indicated complete conversion of starting material to a new product. The mixture was filtered and the filtrate was concentrated to dryness. The residue was purified by column chromatography (SiO₂, Petroleum ether/Ethyl acetate=1:0 to 1:4) to give the title compound (2.02 g, 2.31 mmol, 60.7% yield) as a yellow oil.

LCMS (ES, m/z): 873.8 [M+H]⁺.

### Step 2 of 2: Synthesis of Intermediate 9.2, tert-butyl (2R,5S)-5-(((R)-3-aminopiperidin-1-yl)methyl)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate

To a solution of tert-butyl (2R,5S)-5-(((R)-3-(((benzyloxy)carbonyl)amino)piperidin-1-yl)methyl)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate (1.8 g, 2.06 mmol, 1 eq) in MeOH (120 mL) was added Pd/C (1.5 g, 10% purity) under N₂ atmosphere. The suspension was degassed and purged with H₂ several times. The mixture was stirred under H₂ (15 psi) at 50° C. for 12 h after which time LCMS indicated formation of a product of target mass. The mixture was filtered and the filtrate was concentrated to give crude title compound (1.3 g, 1.76 mmol, 85.5% yield) as a yellow solid.

LCMS (ES, m/z): 739.5 [M+H]⁺. Minor peak of 625.4 [M+H]⁺ corresponding to TBS cleaved product also noted.

### Preparation of Intermediate 10.2, (S)-4-(((2S,5R)-4-(tert-butoxycarbonyl)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxylic acid

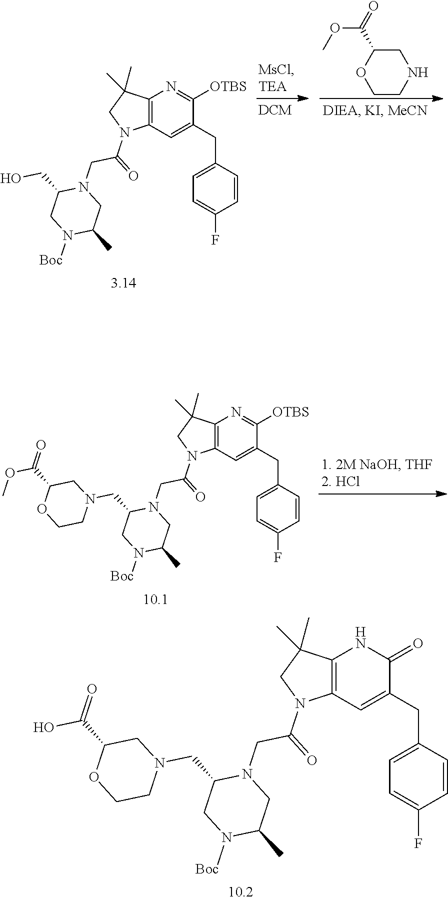

### Step 1 of 2: Synthesis of intermediate 10.1, methyl (S)-4-(((2S,5R)-4-(tert-butoxycarbonyl)-1-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-5-methylpiperazin-2-yl)methyl)morpholine-2-carboxylate

MsCl (290 mg, 2.53 mmol) was added to a solution of (2R,5R)-4-{2-[6-(4-fluorobenzyl)-3,3-dimethyl-2,3-dihydro-pyrrolo[3,2-b]pyridin-1-yl]-2-oxo-ethyl}-5-hydroxymethyl-2-methyl-piperazine-1-carboxylic acid tert-butyl ester (760 mg, 1.45 mmol) and triethylamine (293 mg, 2.89 mmol) in dichloromethane (20 mL) at 0° C. The solution was allowed to warm to room temperature and stirred under a nitrogen atmosphere overnight. The mixture was partitioned between aqueous ammonium chloride and dichloromethane and the isolated organic phase was dried over sodium sulphate, filtered, and evaporated in vacuo. The residue was purified by column chromatography on silica gel eluting with petroleum ether/ethyl acetate (1:1) to give the intermediate product (500 mg, 0.917 mmol, 63.7% yield), as a white solid. To a solution of the intermediate product (5.59 g, 7.61 mmol, 1 eq) in MeCN (100 mL) was added methyl (S)-morpholine-2-carboxylate (1.66 g, 9.13 mmol, 1.2 eq, HCl), DIEA (3.93 g, 30.4 mmol, 5.30 mL, 4 eq) and KI (1.26 g, 7.61 mmol, 1 eq). The mixture was stirred at 80° C. for 12 h after which time LC-MS indicated complete consumption of starting material and formation of a product of target mass. The mixture was concentrated under reduced pressure and the residue was purified by column chromatography (SiO₂, Petroleum ether/Ethyl acetate=I/O to 0/1) to give methyl the title compound (5.9 g, 7.53 mmol, 98% yield) as a yellow oil.

TLC (Petroleum ether/Ethyl acetate=1:2) R_{f}=0.6.

LCMS (ES, m/z): 784.7 [M+H]⁺.

### Step 2 of 2: Synthesis of Intermediate 10.2, (S)-4-(((2S,5R)-4-(tert-butoxycarbonyl)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxylic acid

To a solution of methyl (S)-4-(((2S,5R)-4-(tert-butoxycarbonyl)-1-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-5-methylpiperazin-2-yl)methyl)morpholine-2-carboxylate (5.9 g, 7.53 mmol, 1 eq) in THE (100 mL) was added NaOH (2 M, 7.53 mL, 2 eq). The mixture was stirred at 15° C. for 2 h after which time LCMS indicated consumption of starting material and formation of a product of target mass. The mixture was concentrated to remove most of the THF under reduced pressure. The reaction mixture was diluted with H₂O (10 mL) and 1N HCl was added to adjust the pH to 3-4. The mixture was filtered and the filter cake was dried to give the title compound (5.75 g, crude) as a yellow solid.

LCMS (ES, m/z): 656.5 [M+H]⁺.

### Preparation of Intermediate 11.3, di-tert-butyl 5,5′-(((2S,2'S)-((1,3-phenylenebis(oxy))bis(methylene))bis(morpholine-2,4-diyl))bis(methylene))(2R,2′R,5S,5'S)-bis(4-(2-(6-bromo-3,3-dimethyl-2,3-dihydro-1H-pyrrolo[3,2-b]pyridin-1-yl)-2-oxoethyl)-2-methylpiperazine-1-carboxylate)

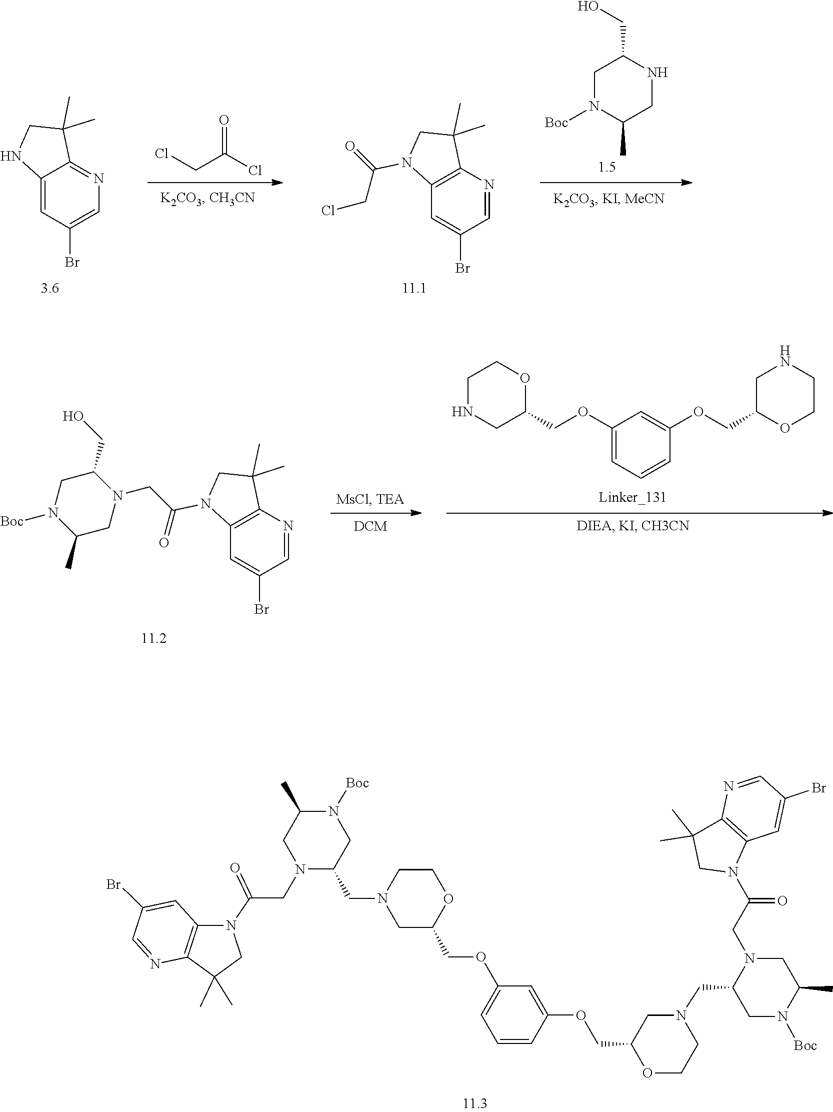

### Step 1 of 3: Synthesis of Intermediate 11.1, 1-(6-bromo-3,3-dimethyl-2,3-dihydro-1H-pyrrolo[3,2-b]pyridin-1-yl)-2-chloroethan-1-one

To a solution of 6-bromo-3,3-dimethyl-1,2-dihydropyrrolo[3,2-b]pyridine (1.5 g, 6.61 mmol, 1 eq) in MeCN (20 mL) was added K₂CO₃ (2.28 g, 16.5 mmol, 2.5 eq) and 2-chloroacetyl chloride (932 mg, 8.26 mmol, 656 μL, 1.25 eq) in MeCN (2 mL) at 0° C. The mixture was stirred at 25° C. for 4 after which time LCMS indicated complete conversion to a product of target mass. MeOH (10 mL) was added to the mixture and stirred at 20° C. for 0.5 h. The mixture was concentrated to dryness and the residue partitioned between H₂O (20 mL) and EtOAc (10 mL). The phases were separated and the aqueous phase was extracted with EtOAc (10 mL*3). The combined organic layers were washed with brine (10 mL), dried over Na₂SO₄, filtered, and concentrated under reduced pressure to give a residue. The residue was purified by column chromatography (SiO₂, Petroleum ether/Ethyl acetate=1:0 to 1:2) to give the title compound (2.00 g, 6.59 mmol, 99% yield) as a white solid.

TLC (Petroleum ether/Ethyl acetate=1:1) R_{f}=0.7

LCMS (ES, m/z): 303.1, 305.1 [M+H]⁺.

### Step 2 of 3: Synthesis of Intermediate 11.2, tert-butyl (2R,5R)-4-(2-(6-bromo-3,3-dimethyl-2,3-dihydro-1H-pyrrolo[3,2-b]pyridin-1-yl)-2-oxoethyl)-5-(hydroxymethyl)-2-methylpiperazine-1-carboxylate

To a solution of 1-(6-bromo-3,3-dimethyl-2H-pyrrolo[3,2-b]pyridin-1-yl)-2-chloro-ethanone (2.00 g, 6.59 mmol, 1 eq) in MeCN (50 mL) were added tert-butyl (2R,5R)-5-(hydroxymethyl)-2-methyl-piperazine-1-carboxylate (1.97 g, 8.56 mmol, 1.3 eq), K₂CO₃(1.82 g, 13.18 mmol, 2 eq) and KI (1.09 g, 6.59 mmol, 1 eq). The mixture was stirred at 50° C. for 12 h after which time LCMS indicated complete conversion to a product of target mass. The mixture was concentrated under reduced pressure and the residue was purified by column chromatography (SiO₂, Petroleum ether/Ethyl acetate=1:0 to 1:2) to give the title compound (2.45 g, 4.93 mmol, 75% yield) as a yellow solid.

TLC (Petroleum ether/Ethyl acetate=1:1) R_{f}=0.5

LCMS (ES, m/z): 497.3, 499.3 [M+H]⁺.

### Step 3 of 3: Synthesis of Intermediate 11.3, di-tert-butyl 5,5′-(((2S,2'S)-((1,3-phenylenebis(oxy))bis(methylene))bis(morpholine-2,4-diyl))bis(methylene))(2R,2′R,5S,5'S)-bis(4-(2-(6-bromo-3,3-dimethyl-2,3-dihydro-1H-pyrrolo[3,2-b]pyridin-1-yl)-2-oxoethyl)-2-methylpiperazine-1-carboxylate)

To a solution of tert-butyl (2R,5R)-4-[2-(6-bromo-3,3-dimethyl-2H-pyrrolo[3,2-b]pyridin-1-yl)-2-oxo-ethyl]-5-(hydroxymethyl)-2-methyl-piperazine-1-carboxylate (2.4 g, 4.82 mmol, 1 eq) in DCM (25 mL) was added DIEA (1.56 g, 12.1 mmol, 2.10 mL, 2.5 eq) and MsCl (829 mg, 7.24 mmol, 560 μL, 1.5 eq) at 0° C. The mixture was stirred at 15° C. for 0.5 h after which time TLC indicated complete consumption of the starting material with formation of a new spot (SiO₂, Petroleum ether/Ethyl acetate=0:1, R_{f}=0.7). The reaction mixture was quenched by addition H₂O (10 mL) and then extracted with DCM (10 mL*3). The combined organic layers were washed with brine (10 mL), dried over Na₂SO₄, filtered, and concentrated under reduced pressure to give the intermediate product as a yellow oil.

The intermediate product was dissolved in MeCN (50 mL) and treated with (2S)-2-[[3-[[(2S)-morpholin-2-yl]methoxy]phenoxy]methyl]morpholine (753 mg, 1.97 mmol, 1.00 eq, 2 HCl), DIEA (1.53 g, 11.85 mmol, 2.06 mL, 6 eq), and KI (656 mg, 3.95 mmol, 2 eq). The mixture was stirred at 80° C. for 12 h after which time LCMS indicated complete conversion to a product of target mass. The mixture was concentrated under reduced pressure and the residue was purified by column chromatography (SiO₂, Ethyl acetate/Methanol=1:0 to 3:1) to give the title compound (1.27 g, 1.00 mmol, 50.6% yield) as a yellow solid.

TLC (Ethyl acetate/Methanol=10:1) R_{f}=0.5

LCMS (ES, m/z): 1265.8, 1267.8, 1269.8 [M+H]⁺.

### V. Preparation of Examples 1-190

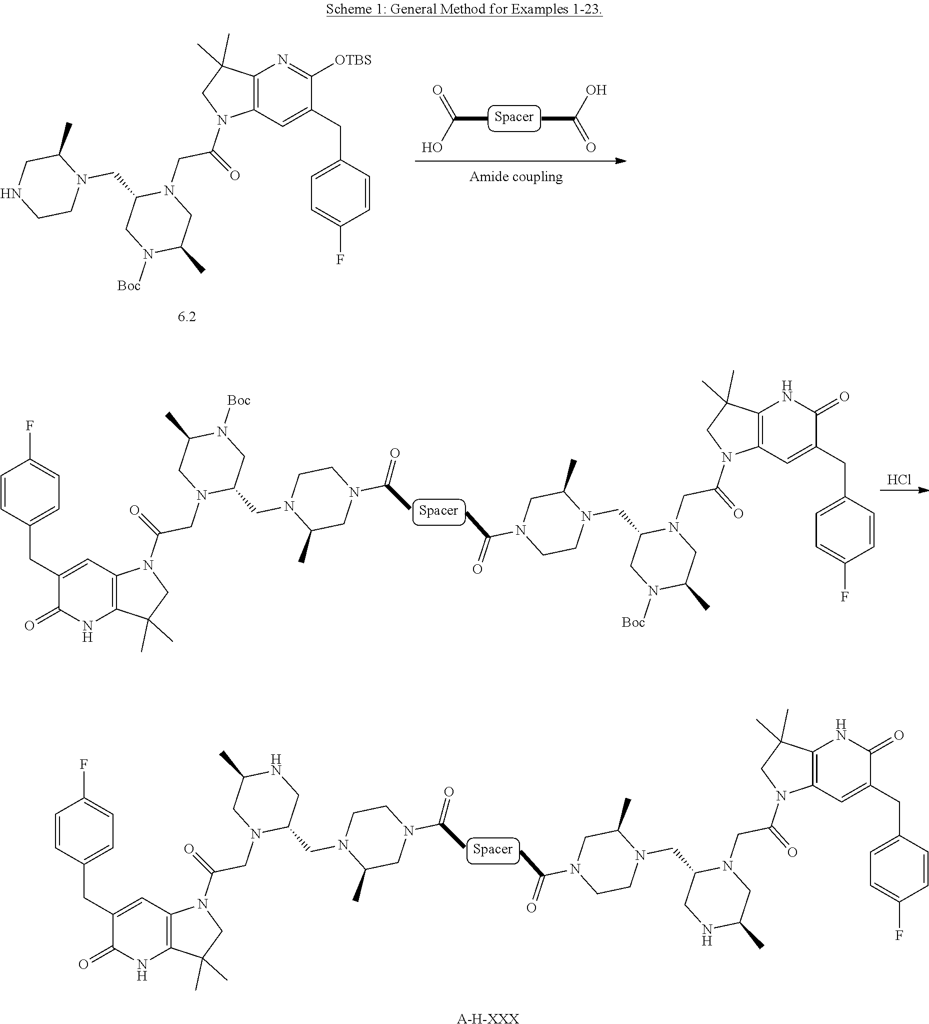

### Example 1, 1,6-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)hexane-1,6-dione

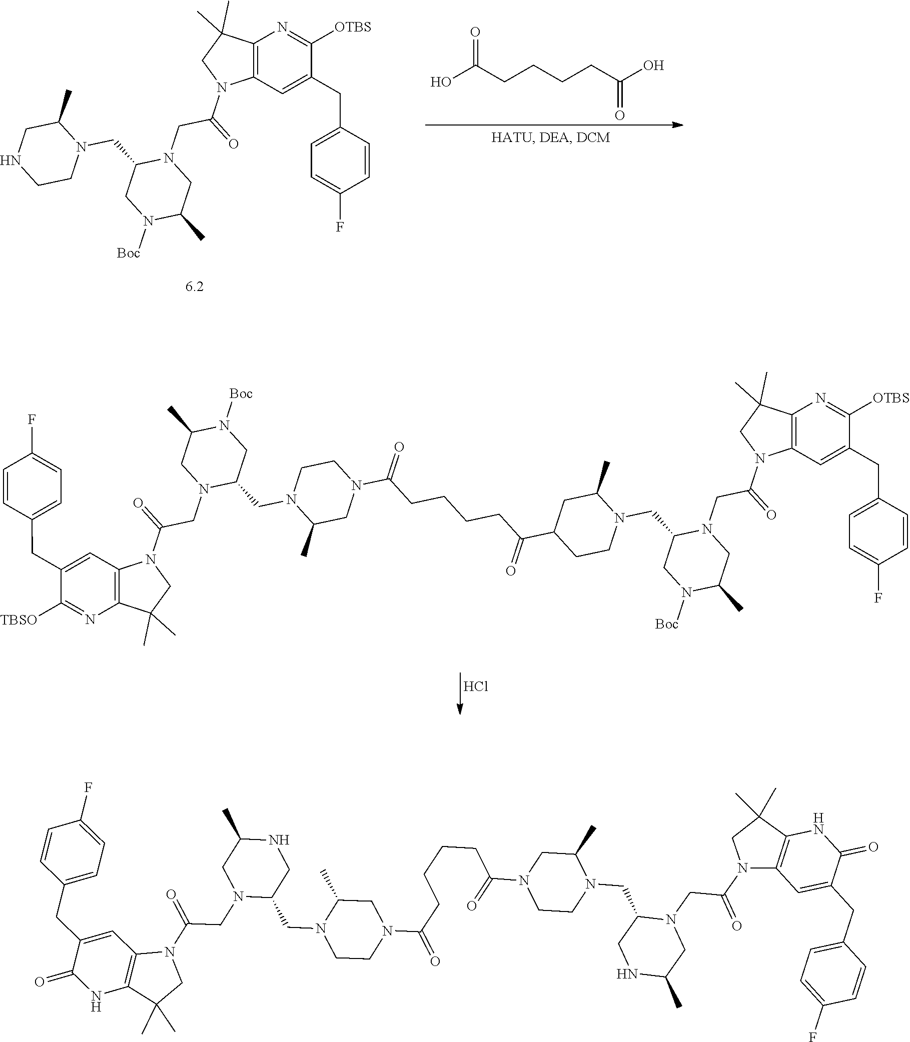

### Step 1 of 2: Synthesis of di-tert-butyl 5,5′-(((2R,2′R)-adipoylbis(2-methylpiperazine-4,1-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate)

HATU (65.1 mg, 171 umol, 2.5 eq) was added to a mixture of tert-butyl (2R,5S)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methyl-5-(((R)-2-methylpiperazin-1-yl)methyl)piperazine-1-carboxylate (Intermediate 6.2) (106 mg, 143 umol, 2.1 eq), adipic acid (10 mg, 68.4 umol, 11.3 uL, 1 eq) and DIEA (35.4 mg, 273 umol, 47.7 uL, 4 eq) in DCM (2 mL) at 15° C. The mixture was stirred at 15° C. for 12 h after which time LCMS indicated conversion to a product of target mass. The mixture was concentrated to dryness and the residue was purified by preparative TLC (Ethyl acetate/MeOH=10/1) to give the title compound (100 mg, 63.0 umol) as a yellow oil.

LCMS (ES, m/z): 1589.1 [M+H]⁺

### Step 2 of 2: Synthesis of 1,6-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)hexane-1,6-dione

A solution of di-tert-butyl 5,5′-(((2R,2′R)-adipoylbis(2-methylpiperazine-4,1-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate) (100 mg, 62.9 umol, 1 eq) in HCl/EtOAc (4M, 2 mL) was stirred at 15° C. for 1 h after which time LCMS indicated conversion to a product of target mass. The precipitate was filtered off and the filter cake was dried to give the crude product. The material was purified by preparative HPLC (FA condition; column: Phenomenex Luna C18 100*30 mm*5 um; mobile phase: [water(0.2% FA)-ACN]; B %: 15%-50%, 9 min) to give the title compound (38.5 mg, 30.5 umol, 48.4% yield, 99% purity, 2 FA) as a white solid.

LCMS (ES, m/z): 1159.7 [M+H]⁺

¹H NMR (400 MHz, METHANOL-d4) δ ppm 8.37-8.47 (m, 2H) 8.10-8.21 (m, 2H) 7.16-7.30 (m, 4H) 7.01 (td, J=8.63, 3.25 Hz, 4H) 3.65-3.96 (m, 15H) 3.40-3.63 (m, 5H) 3.17-3.29 (m, 3H) 2.85-3.11 (m, 11H) 2.76 (brt, J=11.51 Hz, 2H) 2.26-2.49 (m, 6H) 1.97-2.23 (m, 4H) 1.53 (br s, 4H) 1.33-1.42 (m, 12H) 1.26 (d, J=6.50 Hz, 6H) 1.03 (br dd, J=19.26, 5.75 Hz, 6H).

The following compounds (Examples 2-23) were prepared according to the same procedure as Example 1, replacing adipic acid with the appropriate bis-carboxylic acid:

### Example 2, 1,8-bis((R)-4-(((2R,5R)-1-(2-(6-(4-fluorobenzyl)-3,3-dimethyl-5-oxo-2,3,4,5-tetrahydro-1H-Pyrrolo[3,2-b]Pyridin-1-yl)-2-oxoethyl)-5-methylpiperazin-2-yl)methyl)-3-methylpiperazin-1-yl)octane-1,8-dione

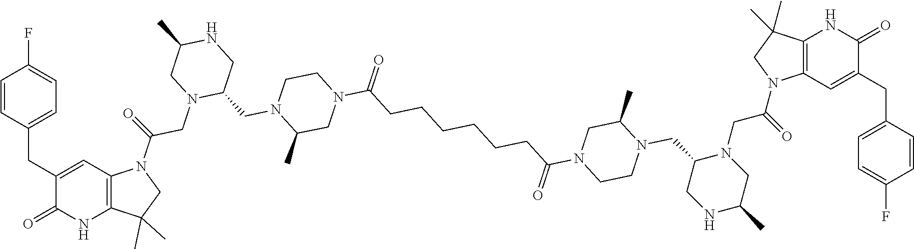 [SMILES: C[C@@H]1CN(CC(=O)N2CC(C)(C)c3[nH]c(=O)c(Cc4ccc(F)cc4)cc32)[C@@H](CN2CCN(C(=O)CCCCCCC(=O)N3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 1187.8 [M+H]P

¹H NMR (400 MHz, CD3OD, 299 K) δ (ppm)=8.19 (br s, 2H), 7.29 (br s, 4H), 7.06 (br t, J=7.7 Hz, 4H), 4.72-4.40 (m, 2H), 4.27-4.00 (m, 10H), 3.94 (br s, 11H), 3.61 (br s, 4H), 3.36 (br d, J=7.8 Hz, 7H), 3.28-2.82 (m, 11H), 2.56 (br s, 5H), 1.73 (br s, 4H), 1.51 (br s, 12H), 1.39 (br t, J=6.5 Hz, 12H).

### Example 3, 1,10-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)decane-1,10-dione

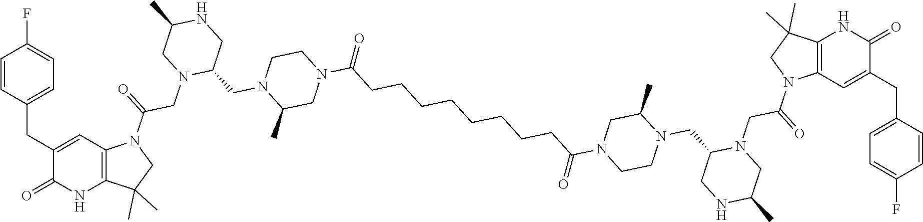 [SMILES: C[C@@H]1CN(CC(=O)N2CC(C)(C)c3[nH]c(=O)c(Cc4ccc(F)cc4)cc32)[C@@H](CN2CCN(C(=O)CCCCCCCCC(=O)N3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 1215.8 [M+H]⁺

¹H NMR (400 MHz, METHANOL-d4) δ=7.93 (br d, J=11.0 Hz, 2H), 7.24 (dd, J=5.6, 8.2 Hz, 4H), 7.01 (t, J=8.8 Hz, 4H), 3.99 (br d, J=8.3 Hz, 3H), 3.90 (br d, J=9.5 Hz, 3H), 3.83 (br s, 4H), 3.80 (br s, 5H), 3.76-3.59 (m, 5H), 3.59-3.37 (m, 7H), 3.30-3.20 (m, 5H), 3.20-2.85 (m, 10H), 2.57-2.45 (m, 4H), 1.69 (br s, 4H), 1.42 (br d, J=6.8 Hz, 20H), 1.33 (br s, 9H), 1.24 (br d, J=5.8 Hz, 3H).

### Example 4, 1,14-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)tetradecane-1,14-dione

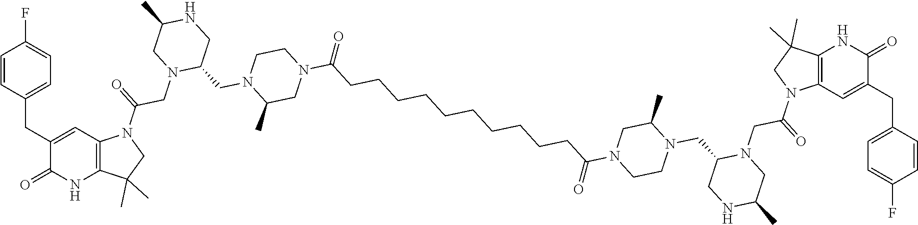 [SMILES: C[C@@H]1CN(CC(=O)N2CC(C)(C)c3[nH]c(=O)c(Cc4ccc(F)cc4)cc32)[C@@H](CN2CCN(C(=O)CCCCCCCCCCC(=O)N3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 1271.9 [M+H]⁺

¹H NMR (400 MHz, METHANOL-d₄) δ=7.94 (br d, J=10.8 Hz, 2H), 7.24 (dd, J=5.6, 8.2 Hz, 4H), 7.01 (t, J=8.7 Hz, 4H), 4.04-3.95 (m, 3H), 3.90 (br d, J=10.1 Hz, 3H), 3.84 (br s, 4H), 3.80 (br s, 5H), 3.78-3.61 (m, 5H), 3.53 (br d, J=6.2 Hz, 7H), 3.30-3.20 (m, 5H), 3.20-2.89 (m, 10H), 2.50 (br t, J=7.2 Hz, 4H), 1.67 (br s, 4H), 1.42 (d, J=6.5 Hz, 16H), 1.33 (br s, 20H), 1.25 (br d, J=5.7 Hz, 4H).

### Example 5, 1,1′-(2,2′-((3R,3′R,6R,6′R)-(((2R,2′R)-(2,2′-(ethane-1,2-diylbis(oxy))bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

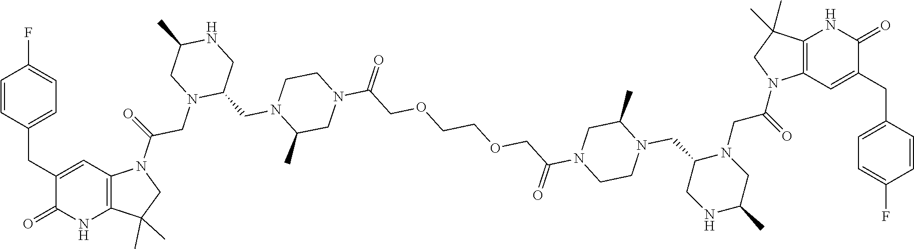 [SMILES: C[C@@H]1CN(CC(=O)N2CC(C)(C)c3[nH]c(=O)c(Cc4ccc(F)cc4)cc32)[C@@H](CN2CCN(C(=O)COCCOCC(=O)N3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 596.6 [M/2+H]³⁰

¹H NMR (400 MHz, METHANOL-d4) δ=7.95 (br s, 2H), 7.28 (br s, 4H), 7.08 (br s, 4H), 4.82-4.31 (m, 6H), 4.12-3.67 (m, 24H), 3.60 (br s, 4H), 3.35 (br s, 4H), 3.30-2.87 (m, 12H), 1.50-1.24 (m, 24H).

### Example 6, 1,1′-(2,2′-((3R,3′R,6R,6′R)-(((2R,2′R)-(2,2′-((oxybis(ethane-2,1-diyl))bis(oxy))bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

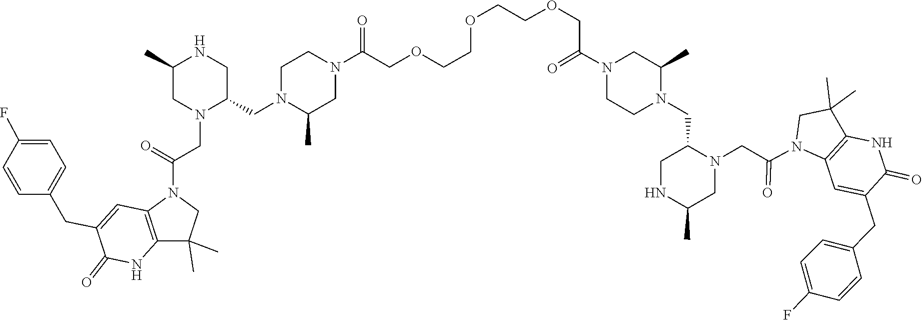 [SMILES: C[C@@H]1CN(CC(=O)N2CC(C)(C)c3[nH]c(=O)c(Cc4ccc(F)cc4)cc32)[C@@H](CN2CCN(C(=O)COCCOCCOCC(=O)N3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 618.6 [M/2+H]⁺

¹H NMR (400 MHz, METHANOL-d4) δ=7.94 (br s, 2H), 7.28-7.20 (m, 4H), 7.03 (s, 4H), 4.50-4.22 (m, 5H), 4.07-3.94 (m, 3H), 3.93-3.78 (m, 11H), 3.73 (br s, 12H), 3.59-3.46 (m, 5H), 3.44-3.32 (m, 4H), 3.25 (br d, J=13.4 Hz, 4H), 3.20-2.84 (m, 10H), 1.42 (d, J=6.4 Hz, 12H), 1.36-1.23 (m, 12H).

### Example 7, 1,1′-(2,2′-((3R,3′R,6R,6′R)-(((2R,2′R)-(2,2′-(1,2-phenylene)bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

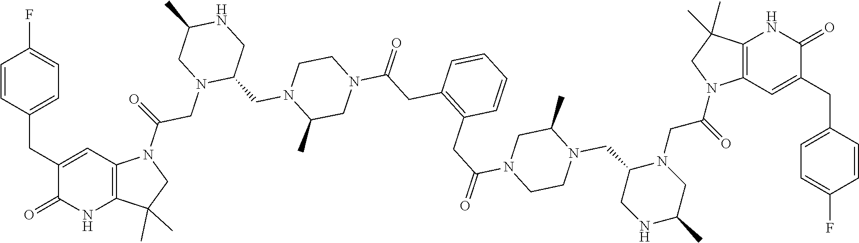 [SMILES: C[C@@H]1CN(CC(=O)N2CC(C)(C)c3[nH]c(=O)c(Cc4ccc(F)cc4)cc32)[C@@H](CN2CCN(C(=O)Cc3ccccc3CC(=O)N3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 604.6 [M/2+H]⁺

¹H NMR (400 MHz, METHANOL-d4) δ=8.52 (s, 2H), 8.14 (br d, J=4.4 Hz, 2H), 7.24-7.16 (m, 4H), 7.16-7.09 (m, 2H), 7.00 (q, J=7.6 Hz, 6H), 3.92-3.83 (m, 3H), 3.80 (br d, J=10.3 Hz, 8H), 3.73-3.63 (m, 7H), 3.61-3.34 (m, 5H), 3.29-3.15 (m, 5H), 3.11-2.67 (m, 14H), 2.47 (br s, 1H), 2.29 (br d, J=3.8 Hz, 1H), 2.18-1.98 (m, 4H), 1.36 (br d, J=15.5 Hz, 12H), 1.25 (br d, J=5.9 Hz, 6H), 1.03-0.94 (m, 6H).

### Example 8, 1,1′-(2,2′-((3R,3′R,6R,6′R)-(((2R,2′R)-(2,2′-(1,2-phenylenebis(oxy))bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

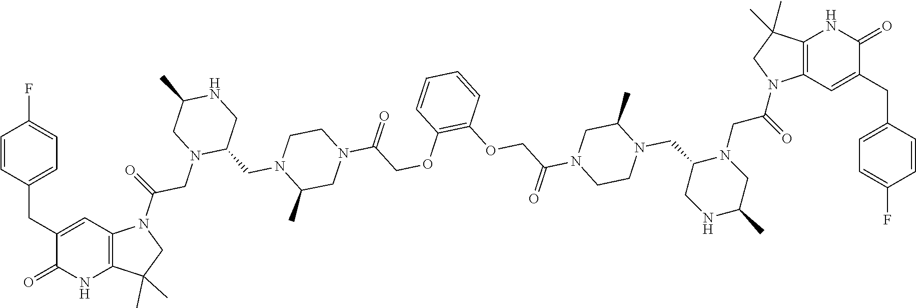 [SMILES: C[C@@H]1CN(CC(=O)N2CC(C)(C)c3[nH]c(=O)c(Cc4ccc(F)cc4)cc32)[C@@H](CN2CCN(C(=O)COc3ccccc3OCC(=O)N3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 620.6 [M/2+H]⁺

¹H NMR (400 MHz, METHANOL-d4) δ=8.54 (s, 2H), 8.15 (br t, J=8.0 Hz, 2H), 7.24-7.16 (m, 4H), 7.04-6.89 (m, 8H), 4.75 (br d, J=4.8 Hz, 6H), 3.92-3.73 (m, 13H), 3.69-3.58 (m, 2H), 3.35 (s, 2H), 3.25-3.11 (m, 4H), 3.08-2.78 (m, 12H), 2.76-2.62 (m, 2H), 2.54-1.90 (m, 7H), 1.37 (br d, J=5.6 Hz, 12H), 1.21 (br s, 6H), 1.07-0.95 (m, 6H).

### Example 9, 1,1′-(2,2′-((3R,3′R,6R,6′R)-(((2R,2′R)-isophthaloylbis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

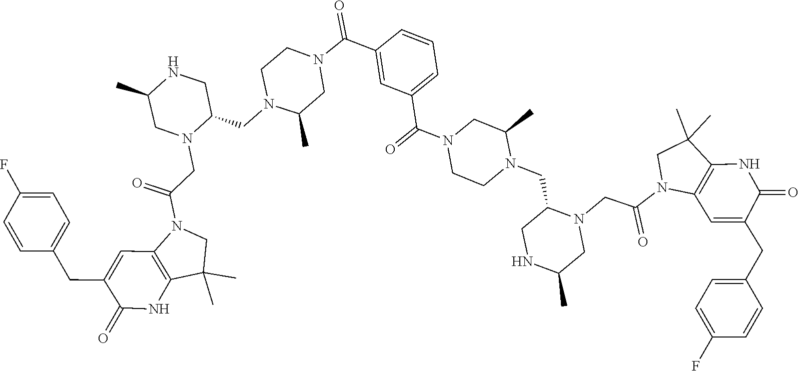 [SMILES: C[C@@H]1CN(CC(=O)N2CC(C)(C)c3[nH]c(=O)c(Cc4ccc(F)cc4)cc32)[C@@H](CN2CCN(C(=O)c3cccc(C(=O)N4CCN(C[C@H]5CN[C@H](C)CN5CC(=O)N5CC(C)(C)c6[nH]c(=O)c(Cc7ccc(F)cc7)cc65)[C@H](C)C4)c3)C[C@H]2C)CN1]

LCMS (ES, m/z): 590.5 [M/2+H]⁺

¹H NMR (400 MHz, METHANOL-d4) δ=8.80-8.33 (m, 2H), 8.27-8.08 (m, 2H), 7.60-7.53 (m, 1H), 7.45 (br d, J=7.3 Hz, 2H), 7.19 (br s, 5H), 6.97 (br s, 4H), 4.04-3.46 (m, 18H), 3.35 (s, 2H), 3.25-3.13 (m, 4H), 3.11-2.57 (m, 14H), 2.49-1.87 (m, 6H), 1.45-1.29 (m, 12H), 1.26 (br s, 6H), 1.12-0.89 (m, 6H).

### Example 10, 1,1′-(2,2′-((3R,3′R,6R,6′R)-(((2R,2′R)-(2,2′-(1,3-phenylene)bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

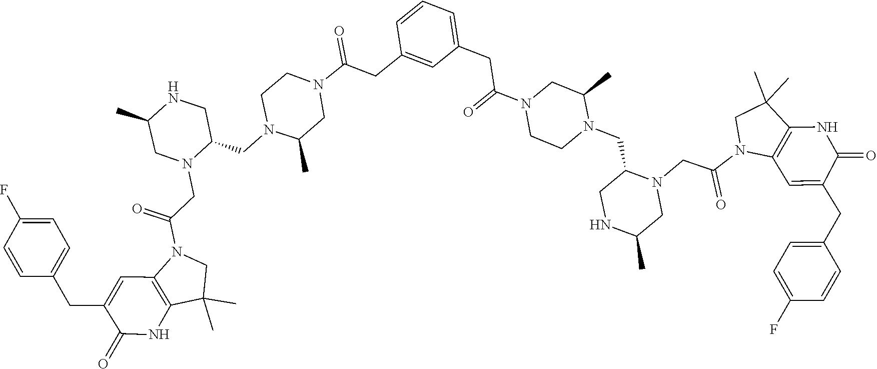 [SMILES: C[C@@H]1CN(CC(=O)N2CC(C)(C)c3[nH]c(=O)c(Cc4ccc(F)cc4)cc32)[C@@H](CN2CCN(C(=O)Cc3cccc(CC(=O)N4CCN(C[C@H]5CN[C@H](C)CN5CC(=O)N5CC(C)(C)c6[nH]c(=O)c(Cc7ccc(F)cc7)cc65)[C@H](C)C4)c3)C[C@H]2C)CN1]

LCMS (ES, m/z): 604.6 [M/2+H]⁺

¹H NMR (400 MHz, METHANOL-d4) δ=8.45 (s, 2H), 8.29-8.17 (m, 2H), 7.26-7.19 (m, 4H), 7.07-6.95 (m, 6H), 6.85-6.59 (m, 2H), 4.10-3.79 (m, 10H), 3.77-3.45 (m, 13H), 3.28-3.08 (m, 8H), 3.07-2.90 (m, 5H), 2.89-2.47 (m, 7H), 2.23-2.03 (m, 3H), 2.00-1.88 (m, 2H), 1.38 (s, 6H), 1.26 (br d, J=6.0 Hz, 7H), 1.20 (br d, J=4.0 Hz, 3H), 1.12 (s, 2H), 1.00-0.92 (m, 6H).

### Example 11, 1,1′-(2,2′-((3R,3′R,6R,6′R)-(((2R,2′R)-(2,2′-(1,3-phenylenebis(oxy))bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

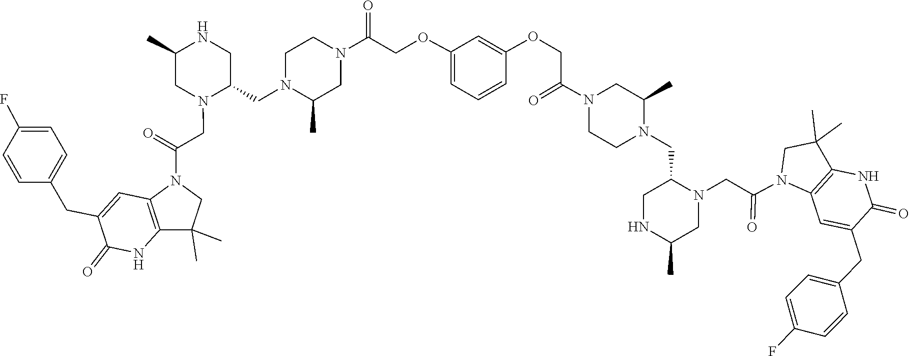 [SMILES: C[C@@H]1CN(CC(=O)N2CC(C)(C)c3[nH]c(=O)c(Cc4ccc(F)cc4)cc32)[C@@H](CN2CCN(C(=O)COc3cccc(OCC(=O)N4CCN(C[C@H]5CN[C@H](C)CN5CC(=O)N5CC(C)(C)c6[nH]c(=O)c(Cc7ccc(F)cc7)cc65)[C@H](C)C4)c3)C[C@H]2C)CN1]

LCMS (ES, m/z): 620.6 [M/2+H]⁺

¹H NMR (400 MHz, METHANOL-d4) δ=8.49 (s, 2H), 8.26-8.13 (m, 2H), 7.22 (br d, J=4.9 Hz, 4H), 7.09-6.89 (m, 5H), 6.51-6.21 (m, 3H), 4.68 (br d, J=9.0 Hz, 4H), 4.04-3.42 (m, 19H), 3.30-3.18 (m, 6H), 3.15-2.87 (m, 11H), 2.73 (br t, J=11.9 Hz, 2H), 2.53-2.35 (m, 2H), 2.24-2.00 (m, 4H), 1.43-1.38 (m, 6H), 1.37-1.30 (m, 6H), 1.25 (d, J=6.4 Hz, 6H), 1.11-0.97 (m, 6H).

### Example 12, 1,1′-(2,2′-((3R,3′R,6R,6′R)-(((2R,2′R)-(2,2′-(1,4-phenylene)bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

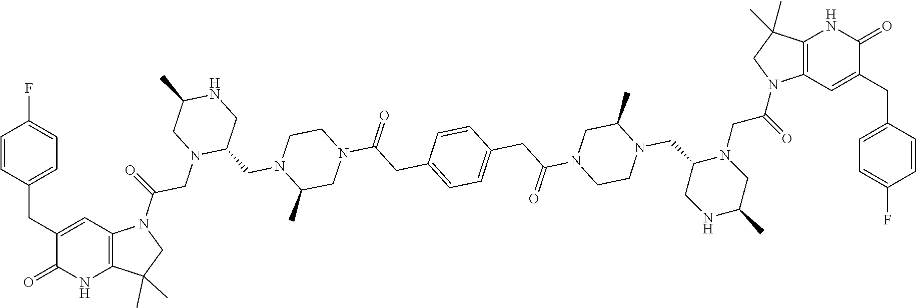 [SMILES: C[C@@H]1CN(CC(=O)N2CC(C)(C)c3[nH]c(=O)c(Cc4ccc(F)cc4)cc32)[C@@H](CN2CCN(C(=O)Cc3ccc(CC(=O)N4CCN(C[C@H]5CN[C@H](C)CN5CC(=O)N5CC(C)(C)c6[nH]c(=O)c(Cc7ccc(F)cc7)cc65)[C@H](C)C4)cc3)C[C@H]2C)CN1]

LCMS (ES, m/z): 604.6 [M/2+H]⁺

¹H NMR (400 MHz, METHANOL-d4) δ=8.46 (br s, 2H), 8.16-8.10 (m, 2H), 7.20 (br t, J=6.2 Hz, 4H), 7.07-6.95 (m, 8H), 3.89-3.76 (m, 9H), 3.75-3.56 (m, 11H), 3.55-3.45 (m, 2H), 3.29-3.15 (m, 6H), 3.13-2.68 (m, 14H), 2.47 (br d, J=6.6 Hz, 1H), 2.30-2.20 (m, 1H), 2.15-1.97 (m, 4H), 1.34 (br s, 6H), 1.26 (dd, J=6.1, 10.0 Hz, 12H), 1.03-0.93 (m, 6H).

### Example 13, 1,1′-(2,2′-((3R,3′R,6R,6′R)-(((2R,2′R)-(2,2′-(1,4-phenylenebis(oxy))bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

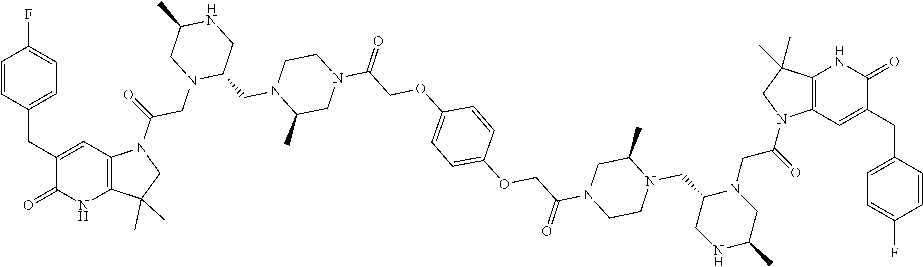 [SMILES: C[C@@H]1CN(CC(=O)N2CC(C)(C)c3[nH]c(=O)c(Cc4ccc(F)cc4)cc32)[C@@H](CN2CCN(C(=O)COc3ccc(OCC(=O)N4CCN(C[C@H]5CN[C@H](C)CN5CC(=O)N5CC(C)(C)c6[nH]c(=O)c(Cc7ccc(F)cc7)cc65)[C@H](C)C4)cc3)C[C@H]2C)CN1]

LCMS (ES, m/z): 620.6 [M/2+H]⁺

¹H NMR (400 MHz, METHANOL-d4) δ=8.44 (s, 2H), 8.20-8.12 (m, 2H), 7.21 (ddd, J=2.8, 5.3, 8.4 Hz, 4H), 6.98 (q, J=8.9 Hz, 4H), 6.79-6.66 (m, 4H), 4.73-4.59 (m, 4H), 4.01-3.42 (m, 19H), 3.29-3.20 (m, 4H), 3.17-3.02 (m, 3H), 3.02-2.84 (m, 8H), 2.74 (br t, J=11.8 Hz, 2H), 2.45 (br d, J=3.1 Hz, 2H), 2.24-2.01 (m, 4H), 1.39 (s, 6H), 1.32 (br d, J=18.7 Hz, 6H), 1.26 (d, J=6.4 Hz, 6H), 1.11-0.96 (m, 6H).

### Example 14, 1,1′-(2,2′-((3R,3′R,6R,6′R)-(((2R,2′R)-((2E,2′E)-3,3′-(1,4-phenylene)bis(acryloyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

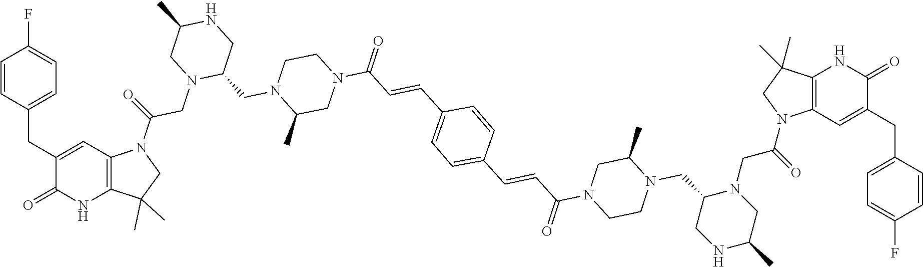 [SMILES: C[C@@H]1CN(CC(=O)N2CC(C)(C)c3[nH]c(=O)c(Cc4ccc(F)cc4)cc32)[C@@H](CN2CCN(C(=O)/C=C/c3ccc(/C=C/C(=O)N4CCN(C[C@H]5CN[C@H](C)CN5CC(=O)N5CC(C)(C)c6[nH]c(=O)c(Cc7ccc(F)cc7)cc65)[C@H](C)C4)cc3)C[C@H]2C)CN1]

LCMS (ES, m/z): 616.5 [M/2+H]⁺

¹H NMR (400 MHz, METHANOL-d4) δ=8.47 (s, 2H), 8.16 (s, 2H), 7.65 (s, 4H), 7.60-7.52 (m, 2H), 7.23 (br s, 4H), 7.15 (br d, J=15.4 Hz, 2H), 7.05-6.95 (m, 4H), 3.91-3.77 (m, 16H), 3.47 (br s, 3H), 3.38 (br d, J=11.7 Hz, 2H), 3.30-3.10 (m, 4H), 3.08-2.91 (m, 9H), 2.79 (br t, J=11.9 Hz, 2H), 2.50 (br s, 2H), 2.32-2.04 (m, 4H), 1.40 (d, J=5.3 Hz, 12H), 1.27 (d, J=6.4 Hz, 6H), 1.07 (br d, J=2.4 Hz, 6H).

### Example 15, 1,1′-(2,2′-((3R,3′R,6R,6′R)-(((2R,2′R)-(2,2′-(methylazanediyl)bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

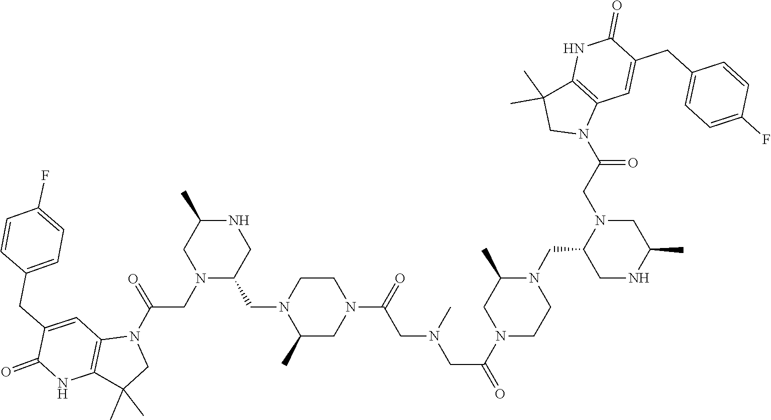 [SMILES: C[C@@H]1CN(CC(=O)N2CC(C)(C)c3[nH]c(=O)c(Cc4ccc(F)cc4)cc32)[C@@H](CN2CCN(C(=O)CN(C)CC(=O)N3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 581.0 [M/2+H]⁺

¹H NMR (400 MHz, METHANOL-d4) δ=8.01 (s, 2H), 7.26 (dd, J=5.6, 8.0 Hz, 4H), 7.06 (t, J=8.8 Hz, 4H), 4.64-4.31 (m, 4H), 4.04-3.79 (m, 13H), 3.79-3.59 (m, 5H), 3.58-3.32 (m, 12H), 3.30-3.16 (m, 4H), 3.15-2.60 (m, 11H), 1.42 (br d, J=6.0 Hz, 12H), 1.37-1.21 (m, 12H).

### Example 16, 1,1′-(2,2′-((3R,3′R,6R,6′R)-(((2R,2′R)-(pyrazine-2,5-dicarbonyl)bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

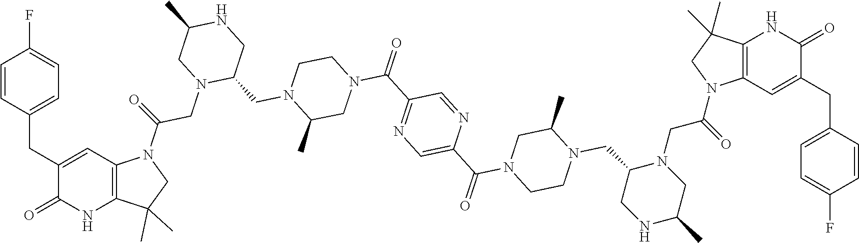 [SMILES: C[C@@H]1CN(CC(=O)N2CC(C)(C)c3[nH]c(=O)c(Cc4ccc(F)cc4)cc32)[C@@H](CN2CCN(C(=O)c3cnc(C(=O)N4CCN(C[C@H]5CN[C@H](C)CN5CC(=O)N5CC(C)(C)c6[nH]c(=O)c(Cc7ccc(F)cc7)cc65)[C@H](C)C4)cn3)C[C@H]2C)CN1]

LCMS (ES, m/z): 591.5 [M/2+H]⁺

¹H NMR (400 MHz, METHANOL-d4) δ=8.78 (d, J=4.4 Hz, 2H), 8.47 (s, 2H), 8.16-8.10 (m, 2H), 7.21 (ddd, J=5.5, 8.5, 11.8 Hz, 4H), 7.04-6.91 (m, 4H), 4.04-3.94 (m, 1H), 3.90-3.70 (m, 14H), 3.64 (br dd, J=2.8, 14.2 Hz, 1H), 3.55-3.45 (m, 3H), 3.41-3.32 (m, 3H), 3.29-3.16 (m, 4H), 3.08-2.84 (m, 10H), 2.77 (br t, J=11.7 Hz, 2H), 2.64 (br s, 1H), 2.48 (br d, J=5.1 Hz, 1H), 2.31-2.08 (m, 4H), 1.39 (s, 12H), 1.26 (dd, J=2.3, 6.1 Hz, 6H), 1.11 (br d, J=6.2 Hz, 3H), 0.97 (br d, J=6.2 Hz, 3H).

### Example 17, 1,1′-(2,2′-((3R,3′R,6R,6′R)-(((2R,2′R)-([2,2′-bipyridine]-3,3′-dicarbonyl)bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

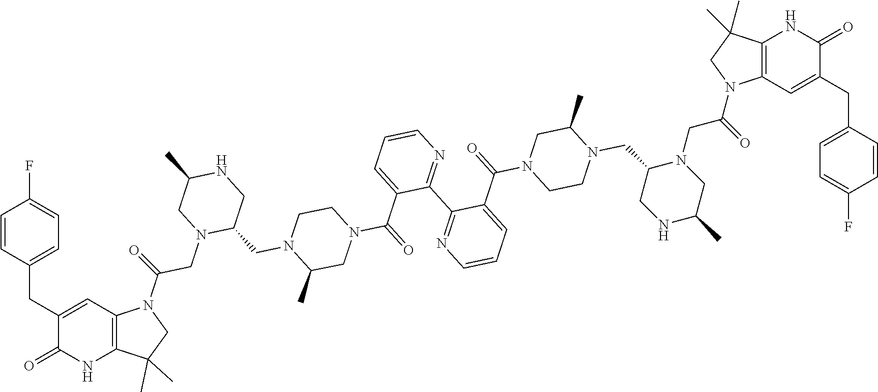 [SMILES: C[C@@H]1CN(CC(=O)N2CC(C)(C)c3[nH]c(=O)c(Cc4ccc(F)cc4)cc32)[C@@H](CN2CCN(C(=O)c3cccnc3-c3ncccc3C(=O)N3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 629.7 [M/2+H]P

¹H NMR (400 MHz, METHANOL-d4) δ=9.03-8.69 (m, 2H), 8.32-7.88 (m, 4H), 7.82-7.42 (m, 2H), 7.32-6.78 (m, 8H), 4.83-4.40 (m, 2H), 4.16-3.68 (m, 20H), 3.65-3.43 (m, 6H), 3.41-3.33 (m, 4H), 3.28-2.53 (m, 10H), 1.44 (br s, 12H), 1.40-1.02 (m, 12H).

### Example 18, 1,1′-(2,2′-((3R,3′R,6R,6′R)-(((2R,2′R)-(naphthalene-2,6-dicarbonyl)bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

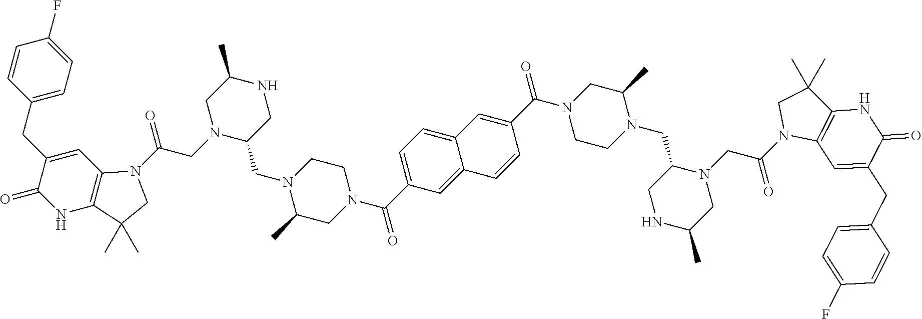 [SMILES: C[C@@H]1CN(CC(=O)N2CC(C)(C)c3[nH]c(=O)c(Cc4ccc(F)cc4)cc32)[C@@H](CN2CCN(C(=O)c3ccc4cc(C(=O)N5CCN(C[C@H]6CN[C@H](C)CN6CC(=O)N6CC(C)(C)c7[nH]c(=O)c(Cc8ccc(F)cc8)cc76)[C@H](C)C5)ccc4c3)C[C@H]2C)CN1]

LCMS (ES, m/z): 615.5 [M/2+H]⁺

¹H NMR (400 MHz, METHANOL-d4) δ=8.51 (s, 2H), 8.12-7.97 (m, 4H), 7.91 (s, 2H), 7.49 (br s, 2H), 7.23-6.66 (m, 8H), 4.09-3.90 (m, 2H), 3.89-3.66 (m, 13H), 3.53-3.36 (m, 4H), 3.29-3.19 (m, 4H), 3.18-2.88 (m, 11H), 2.87-2.63 (m, 4H), 2.62-2.25 (m, 3H), 2.19-2.06 (m, 3H), 1.45-1.38 (m, 12H), 1.24 (br d, J=6.0 Hz, 6H), 1.19-1.11 (m, 3H), 0.85 (br d, J=3.8 Hz, 3H).

### Example 19, 1,1′-(2,2′-((3R,3′R,6R,6′R)-(((2R,2′R)-terephthaloylbis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

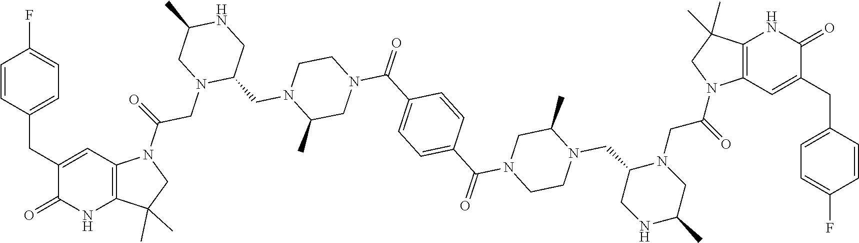 [SMILES: C[C@@H]1CN(CC(=O)N2CC(C)(C)c3[nH]c(=O)c(Cc4ccc(F)cc4)cc32)[C@@H](CN2CCN(C(=O)c3ccc(C(=O)N4CCN(C[C@H]5CN[C@H](C)CN5CC(=O)N5CC(C)(C)c6[nH]c(=O)c(Cc7ccc(F)cc7)cc65)[C@H](C)C4)cc3)C[C@H]2C)CN1]

LCMS (ES, m/z): 590.6 [M/2+H]⁺

¹H NMR (400 MHz, METHANOL-d4) δ=8.53 (s, 2H), 8.12 (br s, 2H), 7.40 (br s, 4H), 7.19 (br d, J=12.2 Hz, 4H), 7.04-6.87 (m, 4H), 4.01-3.68 (m, 14H), 3.53-3.32 (m, 5H), 3.22 (br d, J=6.5 Hz, 4H), 3.13 (br dd, J=2.6, 4.1 Hz, 2H), 2.96 (br s, 8H), 2.83-2.65 (m, 3H), 2.62-2.21 (m, 3H), 2.18-2.06 (m, 3H), 1.39 (s, 12H), 1.23 (br d, J=6.0 Hz, 6H), 1.12 (br d, J=3.5 Hz, 3H), 0.90 (br s, 3H).

### Example 20, 1,1′-(2,2′-((3R,3′R,6R,6′R)-(((2R,2′R)-(4,4′-((E)-ethene-1,2-diyl)bis(benzoyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

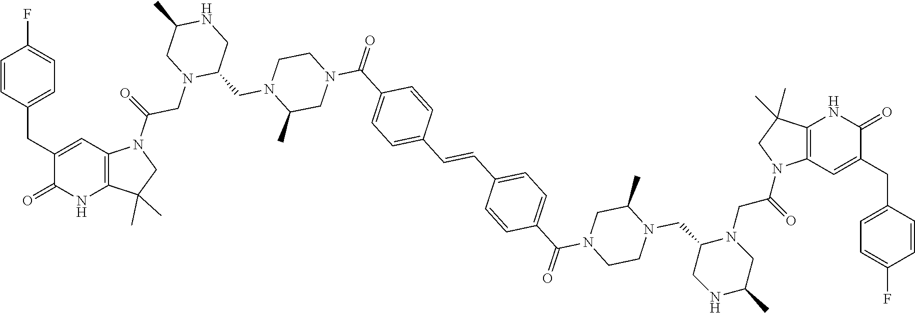 [SMILES: C[C@@H]1CN(CC(=O)N2CC(C)(C)c3[nH]c(=O)c(Cc4ccc(F)cc4)cc32)[C@@H](CN2CCN(C(=O)c3ccc(/C=C/c4ccc(C(=O)N5CCN(C[C@H]6CN[C@H](C)CN6CC(=O)N6CC(C)(C)c7[nH]c(=O)c(Cc8ccc(F)cc8)cc76)[C@H](C)C5)cc4)cc3)C[C@H]2C)CN1]

LCMS (ES, m/z): 641.6 [M/2+H]⁺

¹H NMR (400 MHz, METHANOL-d4) δ=8.51 (s, 2H), 8.08 (br s, 2H), 7.68 (d, J=8.2 Hz, 4H), 7.38-7.29 (m, 6H), 7.26-6.81 (m, 8H), 4.08-3.70 (m, 15H), 3.54-3.35 (m, 5H), 3.30-3.11 (m, 4H), 3.10-2.83 (m, 10H), 2.76 (br t, J=11.8 Hz, 2H), 2.61-2.41 (m, 2H), 2.34-2.06 (m, 4H), 1.41 (d, J=8.8 Hz, 12H), 1.25 (d, J=6.4 Hz, 6H), 1.19-0.84 (m, 6H).

### Example 21, 1,4-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)butane-1,4-dione

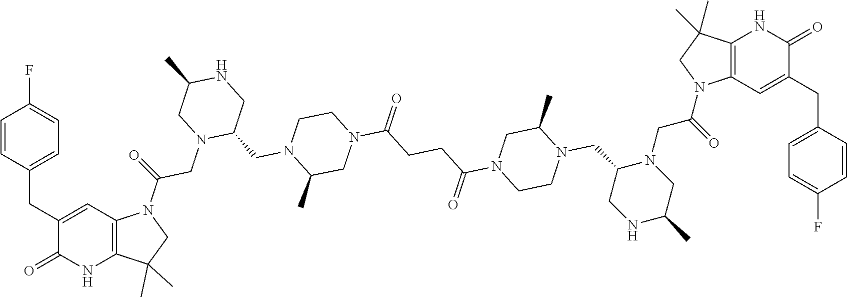 [SMILES: C[C@@H]1CN(CC(=O)N2CC(C)(C)c3[nH]c(=O)c(Cc4ccc(F)cc4)cc32)[C@@H](CN2CCN(C(=O)CCC(=O)N3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 566.6 [M/2+H]⁺

¹H NMR (400 MHz, METHANOL-d4) δ=8.46 (br s, 2H), 8.16 (br s, 2H), 7.28-7.19 (m, 4H), 7.00 (br t, J=8.2 Hz, 4H), 3.91-3.60 (m, 16H), 3.49 (br d, J=1.8 Hz, 3H), 3.34 (br s, 1H), 3.28 (br d, J=6.2 Hz, 3H), 3.08-2.86 (m, 11H), 2.76 (br t, J=11.7 Hz, 2H), 2.64-2.38 (m, 6H), 2.25-2.03 (m, 4H), 1.38 (s, 12H), 1.26 (d, J=6.4 Hz, 6H), 1.10-0.97 (m, 6H).

### Example 22, 1,5-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)pentane-1,5-dione

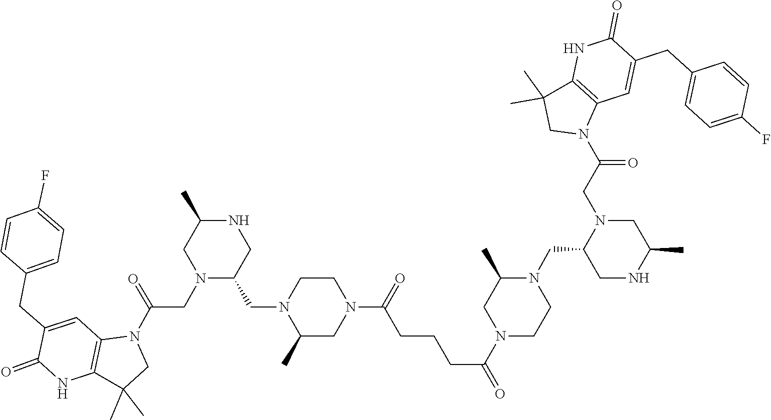 [SMILES: C[C@@H]1CN(CC(=O)N2CC(C)(C)c3[nH]c(=O)c(Cc4ccc(F)cc4)cc32)[C@@H](CN2CCN(C(=O)CCCC(=O)N3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 573.6 [M/2+H]P

¹H NMR (400 MHz, METHANOL-d4) δ=8.38 (s, 2H), 8.23-8.13 (m, 2H), 7.27-7.17 (m, 4H), 7.07-6.96 (m, 4H), 4.05-3.70 (m, 13H), 3.70-3.42 (m, 6H), 3.29-3.05 (m, 6H), 3.04-2.80 (m, 8H), 2.80-2.66 (m, 3H), 2.55-2.25 (m, 6H), 2.23-2.07 (m, 2H), 2.06-1.88 (m, 2H), 1.77 (br s, 2H), 1.39 (br d, J=4.0 Hz, 6H), 1.36-1.25 (m, 12H), 1.06 (br t, J=5.7 Hz, 3H), 0.98 (br t, J=7.3 Hz, 3H).

### Example 23, 1,1′-(2,2′-((3R,3′R,6R,6′R)-(((2R,2′R)-([2,2′-bipyridine]-4,4′-dicarbonyl)bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

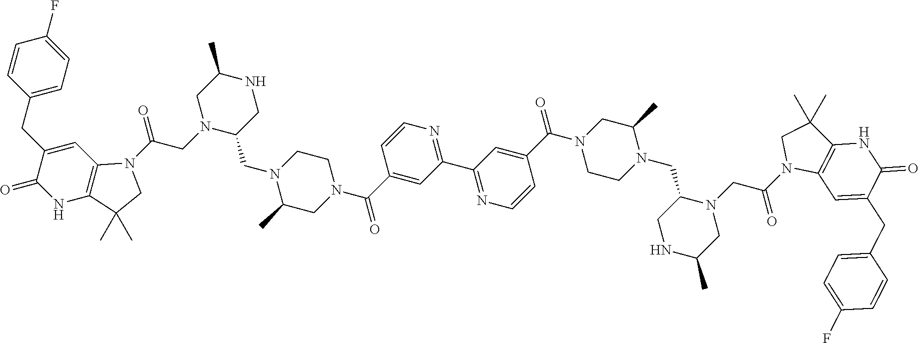 [SMILES: C[C@@H]1CN(CC(=O)N2CC(C)(C)c3[nH]c(=O)c(Cc4ccc(F)cc4)cc32)[C@@H](CN2CCN(C(=O)c3ccnc(-c4cc(C(=O)N5CCN(C[C@H]6CN[C@H](C)CN6CC(=O)N6CC(C)(C)c7[nH]c(=O)c(Cc8ccc(F)cc8)cc76)[C@H](C)C5)ccn4)c3)C[C@H]2C)CN1]

LCMS (ES, m/z): 629.5 [M/2+H]⁺

¹H NMR (400 MHz, METHANOL-d4) δ=8.77 (br d, J=4.3 Hz, 2H), 8.53 (br s, 2H), 8.34 (br s, 2H), 8.12 (br s, 1H), 8.04 (s, 1H), 7.39 (br s, 2H), 7.19 (br s, 2H), 7.10 (br s, 2H), 6.95 (br t, J=8.1 Hz, 2H), 6.83 (br t, J=8.3 Hz, 2H), 3.97-3.69 (m, 14H), 3.51-3.35 (m, 4H), 3.20 (br s, 6H), 3.08-2.67 (m, 12H), 2.60 (br s, 1H), 2.45 (br s, 1H), 2.30 (br s, 1H), 2.14 (br d, J=12.6 Hz, 3H), 1.47-1.34 (m, 12H), 1.22 (br s, 6H), 1.14 (br d, J=5.8 Hz, 3H), 0.91 (br s, 3H).

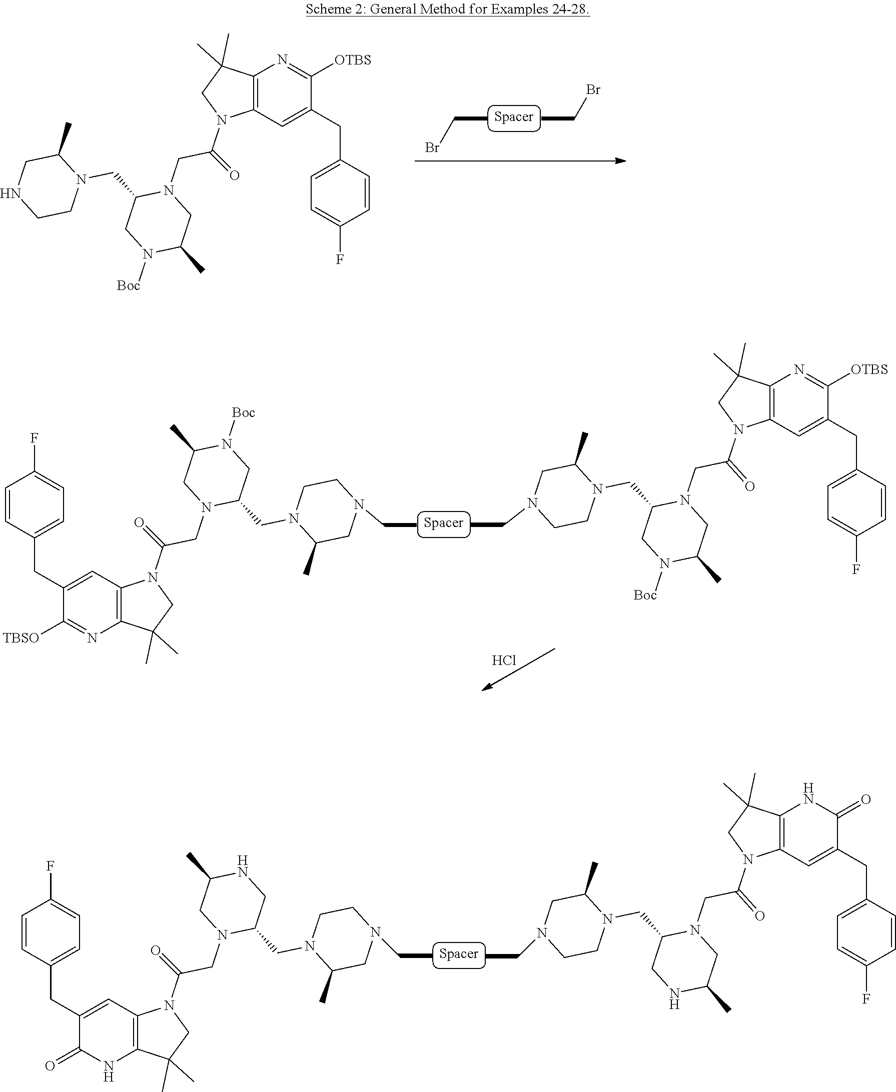

### Example 24, 1,1′-(2,2′-((3R,3′R,6R,6′R)-(((2R,2′R)-octane-1,8-diylbis(2-methyl piperazine-4,1-diyl))bis(methylene))bis(3-methyl piperazine-6,1-diyl))bis(acetyl))bis(6-(4-fluorobenzyl)-3,3-dimethyl-1,2,3,4-tetrahydro-5H1-pyrrolo[3,2-b]pyridin-5-one)

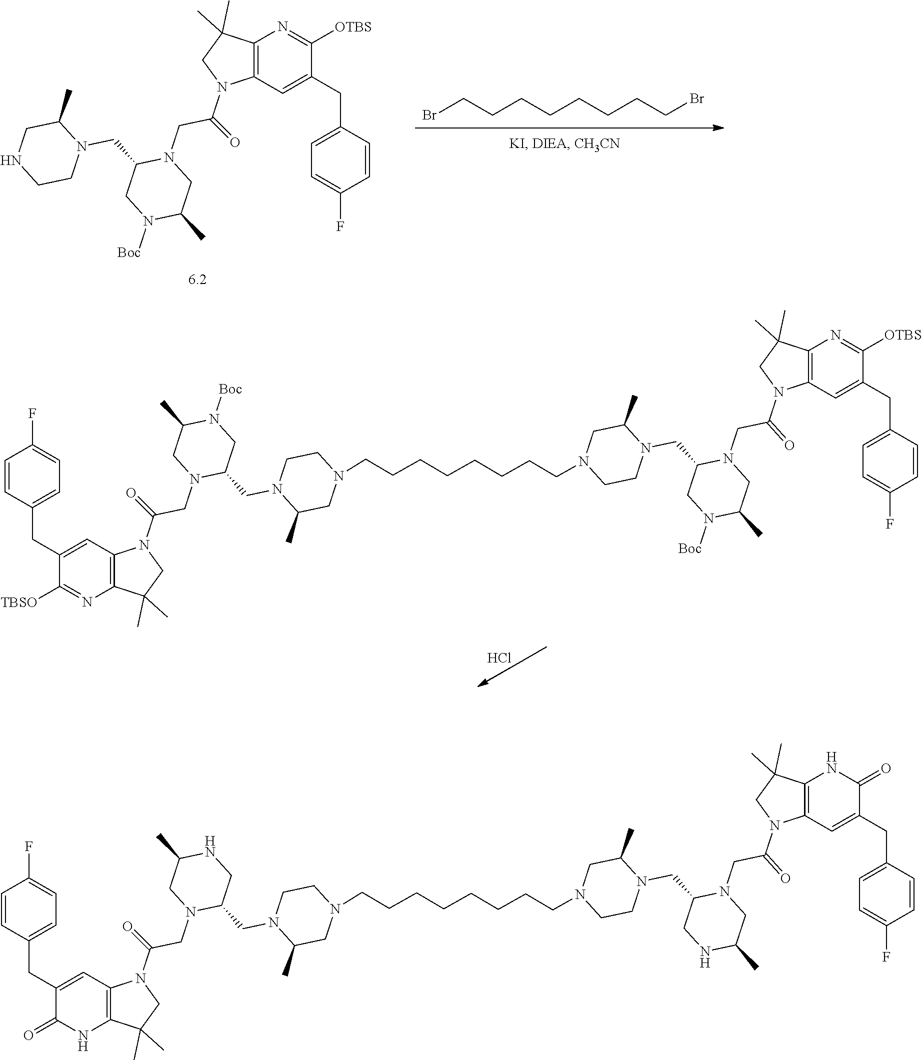

### Step 1 of 2: Synthesis of di-tert-butyl 5,5′-(((2R,2′R)-octane-1,8-diylbis(2-methylpiperazine-4,1-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate)

A mixture of tert-butyl (2R,5S)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methyl-5-(((R)-2-methylpiperazin-1-yl)methyl)piperazine-1-carboxylate (Intermediate 6.2) (149 mg, 202 umol, 2.2 eq), 1,8-dibromooctane (25 mg, 91.9 umol, 17.0 uL, 1 eq), KI (30.5 mg, 184 umol, 2 eq) and DIEA (47.5 mg, 367 umol, 64.0 uL, 4 eq) in CH₃CN (3 mL) was stirred at 45° C. for 24 h after which time LCMS indicated complete conversion to a product of target mass. The mixture was concentrated to dryness and the residue was purified by preparative TLC (Petroleum ether/ethyl acetate/NH₃·H2O=100:10:4) to give the title compound (86 mg, 54.1 umol, 58.9% yield) as a yellow oil.

LCMS (ES, m/z): 1589.1 [M+H]⁺

### Step 2 of 2: Synthesis of 1,1′-(2,2′-((3R,3′R,6R,6′R)-(((2R,2′R)-octane-1,8-diylbis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

To a solution of di-tert-butyl 5,5′-(((2R,2′R)-octane-1,8-diylbis(2-methylpiperazine-4,1-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate) (86 mg, 54.1 umol, 1 eq) in EtOAc (1.5 mL) was added HCl/EtOAc (4 M, 1.5 mL) dropwise at 15° C. The mixture was stirred at 15° C. for 2 h after which time LCMS indicated complete conversion to a product of target mass. The mixture was filtered to give the crude product. This material was purified by preparative HPLC (FA condition; column: Phenomenex Luna C18 200*40 mm*10 um; mobile phase: [water(0.2% FA)-ACN]; B %: 10%-50%, 8 min) to give the title compound (28.6 mg, 22.8 umol, 42.2% yield, 100% purity, 2 FA) as a white solid.

LCMS (ES, m/z): 580.6 [M/2+H]⁺

¹H NMR (400 MHz, METHANOL-d4) δ=8.52 (br s, 2H), 8.38 (s, 2H), 7.34 (dd, J=5.6, 7.9 Hz, 4H), 6.98 (t, J=8.6 Hz, 4H), 4.20 (br d, J=18.1 Hz, 2H), 4.00 (d, J=14.2 Hz, 2H), 3.93-3.79 (m, 4H), 3.78-3.64 (m, 4H), 3.56 (br d, J=17.6 Hz, 2H), 3.44-3.33 (m, 2H), 3.29-2.99 (m, 9H), 2.99-2.92 (m, 2H), 2.83-2.63 (m, 4H), 2.51 (br d, J=3.5 Hz, 2H), 2.39-2.09 (m, 4H), 2.08-1.76 (m, 9H), 1.52 (d, J=19.8 Hz, 12H), 1.27 (br d, J=6.3 Hz, 6H), 1.12 (br d, J=5.8 Hz, 8H), 1.03-0.72 (m, 10H). Examples 25-28 were prepared according to the same procedure as Example 24, replacing 1,8-dibromooctane with the appropriate di-bromide. The compounds of Examples 25-28 were found to have characterizing data as set forth below.

### Example 25, 1,1′-(2,2′-((3R,3′R,6R,6′R)-(((2R,2′R)-decane-1,10-diylbis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

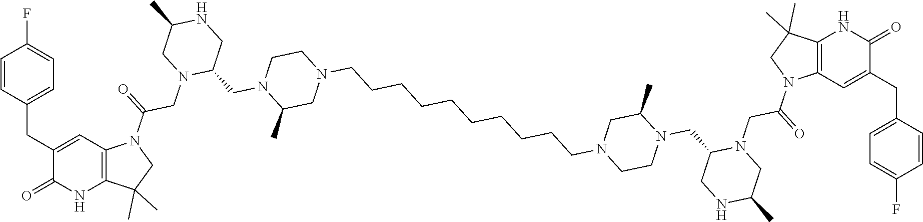 [SMILES: C[C@@H]1CN(CC(=O)N2CC(C)(C)c3[nH]c(=O)c(Cc4ccc(F)cc4)cc32)[C@@H](CN2CCN(CCCCCCCCCCN3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 594.7 [M/2+H]⁺

¹H NMR (400 MHz, METHANOL-d4) δ=8.54 (s, 2H), 8.28 (s, 2H), 7.37-7.25 (m, 4H), 6.99 (t, J=8.8 Hz, 4H), 4.07 (br d, J=17.4 Hz, 2H), 3.97-3.60 (m, 12H), 3.23-2.89 (m, 12H), 2.75-2.48 (m, 6H), 2.36-1.70 (m, 14H), 1.50 (s, 6H), 1.43 (s, 6H), 1.40-1.34 (m, 1H), 1.27-1.03 (m, 27H).

### Example 26, 1,1′-(2,2′-((3R,3′R,6R,6′R)-(((2R,2′R)-dodecane-1,12-diylbis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

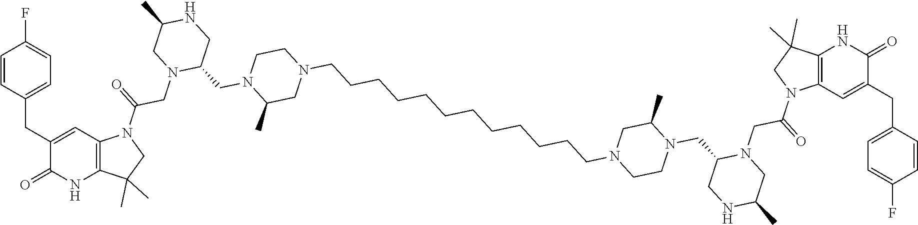 [SMILES: C[C@@H]1CN(CC(=O)N2CC(C)(C)c3[nH]c(=O)c(Cc4ccc(F)cc4)cc32)[C@@H](CN2CCN(CCCCCCCCCCCCN3CCN(C[C@H]4CN[C@H](C)CN4CC(=O)N4CC(C)(C)c5[nH]c(=O)c(Cc6ccc(F)cc6)cc54)[C@H](C)C3)C[C@H]2C)CN1]

LCMS (ES, m/z): 608.7 [M/2+H]⁺

¹H NMR (400 MHz, METHANOL-d4) δ=8.50 (s, 2H), 8.21 (s, 2H), 7.28 (dd, J=5.5, 8.4 Hz, 4H), 7.00 (t, J=8.8 Hz, 4H), 3.98-3.84 (m, 6H), 3.78 (s, 3H), 3.75-3.67 (m, 3H), 3.60 (br s, 2H), 3.26 (br d, J=9.1 Hz, 4H), 3.18-3.03 (m, 6H), 2.97 (br d, J=10.1 Hz, 6H), 2.75 (br t, J=10.8 Hz, 2H), 2.56-1.93 (m, 14H), 1.48-1.34 (m, 14H), 1.32-1.06 (m, 30H).

### Example 27, 1,1′-(2,2′-((3R,3′R,6R,6′R)-(((2R,2′R)-(1,4-phenylenebis(methylene))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

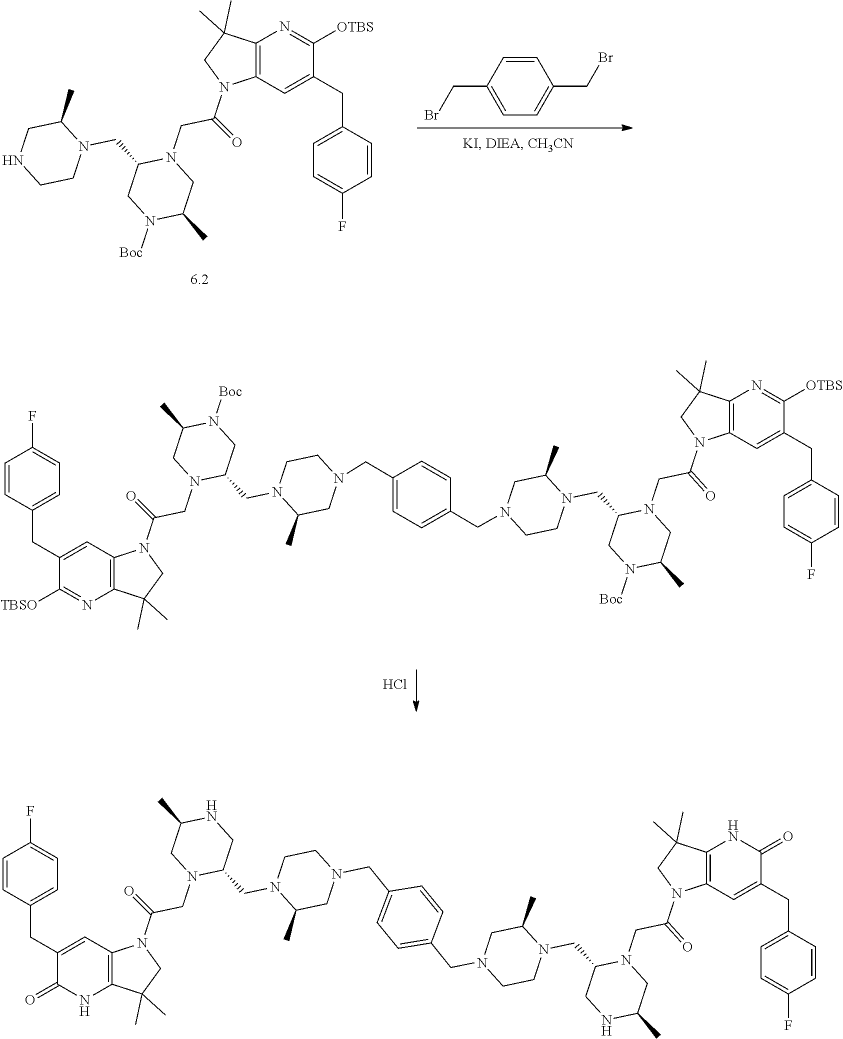

LCMS (ES, m/z): 576.5 [M/2+H]⁺

¹H NMR (400 MHz, METHANOL-d4) δ=8.41 (s, 2H), 8.22 (s, 2H), 7.30-7.24 (m, 4H), 7.02-6.93 (m, 8H), 4.07-3.90 (m, 4H), 3.83-3.68 (m, 6H), 3.66-3.55 (m, 4H), 3.42 (br d, J=13.1 Hz, 2H), 3.27-3.13 (m, 8H), 3.08-2.90 (m, 6H), 2.77-2.47 (m, 6H), 2.35-2.19 (m, 2H), 2.11-1.85 (m, 6H), 1.70 (br s, 2H), 1.41 (s, 6H), 1.29-1.22 (m, 12H), 1.00 (d, J=6.2 Hz, 6H).

### Example 28, 1,1′-(2,2′-((3R,3′R,6R,6′R)-(((2R,2′R)-((perfluoro-1,4-phenylene)bis(methylene))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo [3,2-b]pyridin-5-one)

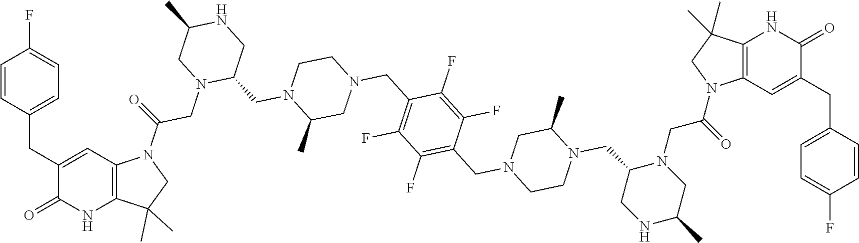 [SMILES: C[C@@H]1CN(CC(=O)N2CC(C)(C)c3[nH]c(=O)c(Cc4ccc(F)cc4)cc32)[C@@H](CN2CCN(Cc3c(F)c(F)c(CN4CCN(C[C@H]5CN[C@H](C)CN5CC(=O)N5CC(C)(C)c6[nH]c(=O)c(Cc7ccc(F)cc7)cc65)[C@H](C)C4)c(F)c3F)C[C@H]2C)CN1]

LCMS (ES, m/z): 612.5 [M/2+H]⁺

¹H NMR (400 MHz, METHANOL-d4) δ=8.51 (s, 2H), 8.25 (s, 2H), 7.33 (dd, J=5.5, 8.4 Hz, 4H), 7.02 (br t, J=8.8 Hz, 4H), 4.58-4.52 (m, 2H), 4.16 (br d, J=17.6 Hz, 2H), 3.98-3.90 (m, 2H), 3.85-3.73 (m, 6H), 3.60-3.45 (m, 6H), 3.35 (s, 2H), 3.19-2.89 (m, 12H), 2.69-2.40 (m, 6H), 2.20 (br d, J=7.4 Hz, 2H), 1.99-1.59 (m, 8H), 1.43 (s, 5H), 1.25 (br d, J=6.4 Hz, 6H), 1.16-0.98 (m, 11H).

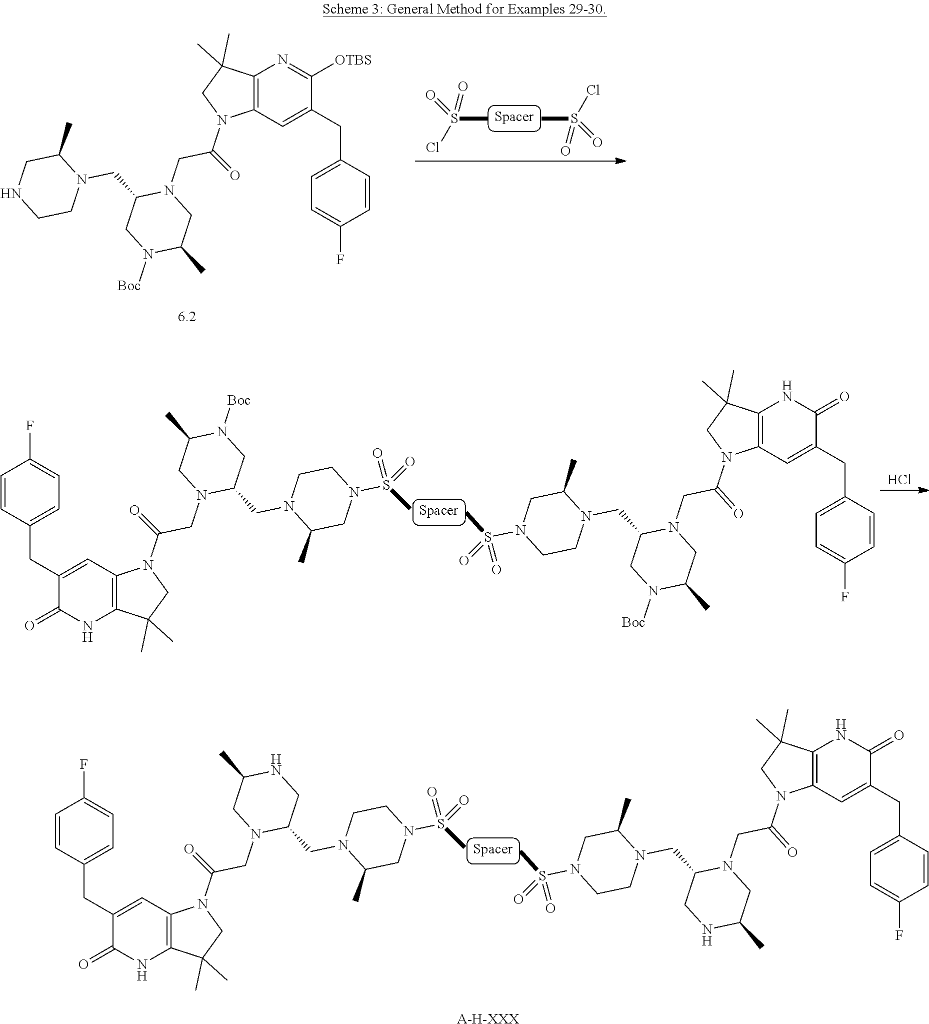

### Example 29, 1,1′-(2,2′-((3R,3′R,6R,6′R)-(((2R,2′R)-([1,1′-biphenyl]-4,4′-disulfonyl)bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

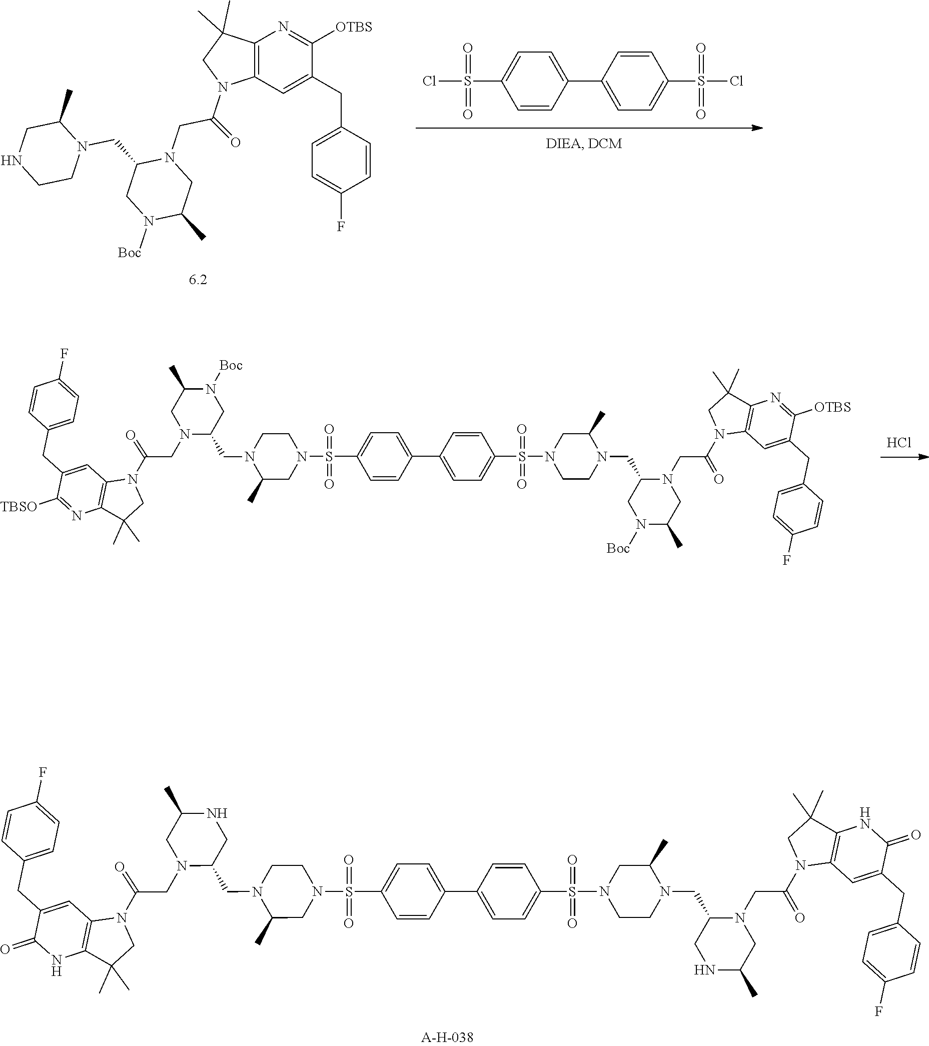

### Step 1 of 2: Synthesis of di-tert-butyl 5,5′-(((2R,2′R)-([1,1′-biphenyl]-4,4′-disulfonyl)bis(2-methylpiperazine-4,1-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate)

4-(4-chlorosulfonylphenyl)benzenesulfonyl chloride (21.6 mg, 61.5 umol, 1 eq) was added to a mixture of tert-butyl (2R,5S)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methyl-5-(((R)-2-methylpiperazin-1-yl)methyl)piperazine-1-carboxylate (Intermediate 6.2) (100 mg, 135 umol, 2.2 eq) and DIEA (39.7 mg, 307 umol, 53.5 uL, 5 eq) in DCM (2 mL) at 15° C. The mixture was stirred at 35° C. for 2 h after which time LCMS indicated complete conversion to a product of target mass. The mixture was concentrated to dryness and the crude product was purified by preparative TLC (SiO₂, Petroleum ether/Ethyl acetate=1:2) to give the title compound (85 mg, 48.4 umol, 78.7% yield) as a yellow solid.

LCMS (ES, m/z): 1757.0 [M+H]⁺

### Step 2 of 2: Synthesis of 1,1′-(2,2′-((3R,3′R,6R,6′R)-(((2R,2′R)—([1,1′-biphenyl]-4,4′-disulfonyl)bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

A solution of di-tert-butyl 5,5′-(((2R,2′R)—([1,1′-biphenyl]-4,4′-disulfonyl)bis(2-methylpiperazine-4,1-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate) (85 mg, 48.4 umol, 1 eq) in HCl/EtOAc (3 M, 2 mL) was stirred at 15° C. for 1 h after which time LCMS indicated complete conversion to a product of target mass. The precipitate was filtered off and the filter cake was dried to give the title compound (28.1 mg, 19.8 umol, 41% yield, 98% purity, 2 HCl) as a white solid.

LCMS (ES, m/z): 1327.6 [M+H]⁺

¹H NMR (400 MHz, DEUTERIUM OXIDE) 6 ppm 7.79 (br s, 2H) 7.60 (br s, 4H) 7.00-7.30 (m, 4H) 6.61-6.90 (m, 8H) 3.63-4.22 (m, 18H) 3.53 (br s, 4H) 2.94-3.40 (m, 18H) 2.22-2.85 (m, 2H) 1.12-1.50 (m, 24H).

Example 30 was prepared according to the same procedure as Example 29, replacing 4-(4-chlorosulfonylphenyl)benzenesulfonyl chloride with 4,4′-oxydibenzenesulfonyl chloride: Example 30 was found to have characterizing data as set forth below.

### Example 30, 2,2′-((3R,3′R,6R,6′R)-(((2R,2′R)-(oxybis(4,1-phenylenesulfonyl))bis(2-methylpiperazine-4,1-diyl))bis(methylene))bis(3-methylpiperazine-6,1-diyl))bis(1-(6-(4-fluorobenzyl)-3,3-dimethyl-5-oxo-2,3,4,5-tetrahydro-1H-pyrrolo[3,2-b]pyridin-1-yl)ethan-1-one)

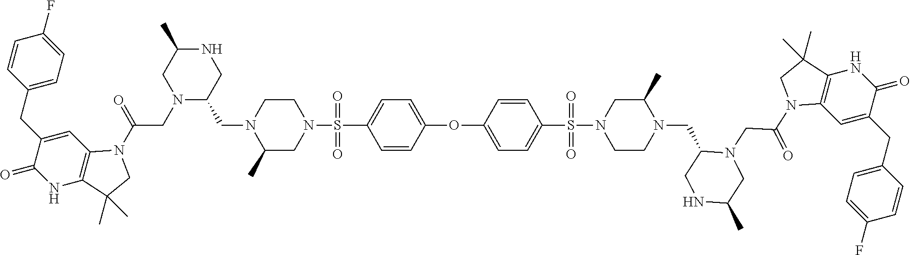 [SMILES: C[C@@H]1CN(CC(=O)N2CC(C)(C)c3[nH]c(=O)c(Cc4ccc(F)cc4)cc32)[C@@H](CN2CCN(S(=O)(=O)c3ccc(Oc4ccc(S(=O)(=O)N5CCN(C[C@H]6CN[C@H](C)CN6CC(=O)N6CC(C)(C)c7[nH]c(=O)c(Cc8ccc(F)cc8)cc76)[C@H](C)C5)cc4)cc3)C[C@H]2C)CN1]

LCMS (ES, m/z): 1343.7 [M+H]⁺

¹H NMR (400 MHz, METHANOL-d4) δ ppm 8.04 (br s, 2H) 7.88 (br d, J=2.50 Hz, 4H) 7.18 (br s, 8H) 6.98 (br t, J=8.25 Hz, 4H) 3.88-4.07 (m, 10H) 3.65-3.86 (m, 12H) 3.47-3.63 (m, 6H) 3.37 (br s, 4H) 2.98-3.29 (m, 10H) 1.42 (br d, J=6.00 Hz, 18H) 1.35 (br d, J=6.00 Hz, 6H)

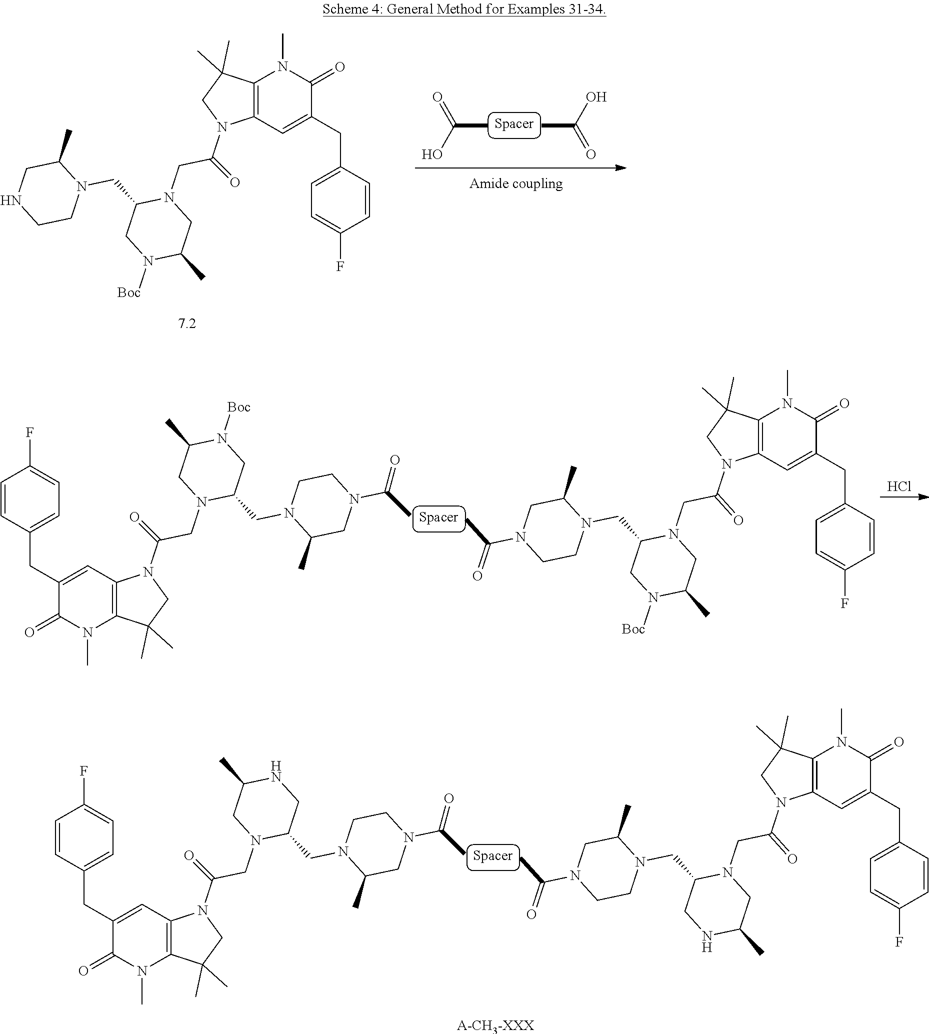

### Example 31, 1,14-bis((R)-4-(((2R,5R)-1-(2-(6-(4-fluorobenzyl)-3,3,4-trimethyl-5-oxo-2,3,4,5-tetrahydro-1H-Pyrrolo[3,2-b]pyridin-1-yl)-2-oxoethyl)-5-methylpiperazin-2-yl)methyl)-3-methylpiperazin-1-yl)tetradecane-1,14-dione

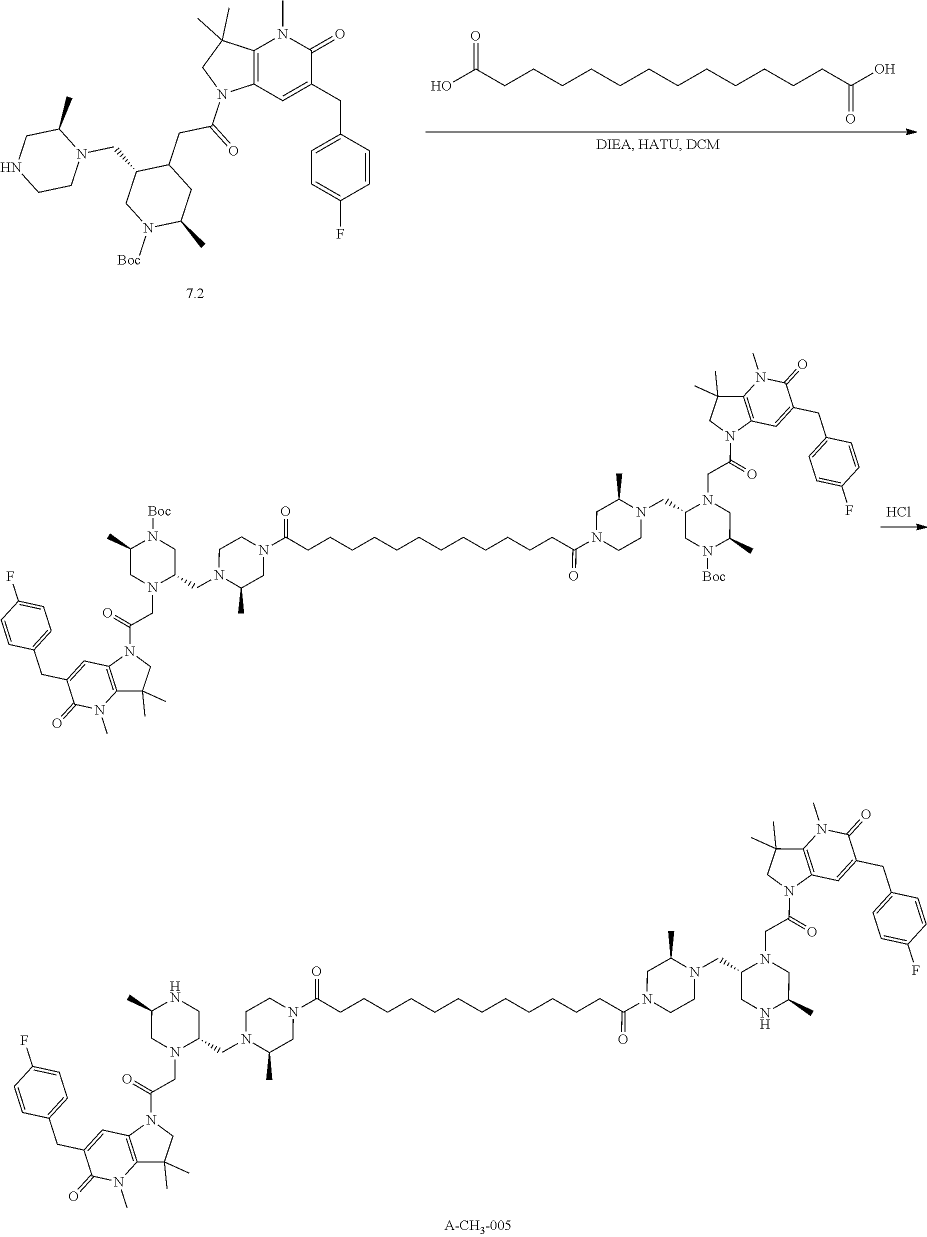

### Step 1 of 2: Synthesis of di-tert-butyl 5,5′-(((2R,2′R)-tetradecanedioylbis(2-methylpiperazine-4,1-diyl))bis(methylene))(2R,2′R,5S,5'S)-bis(4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-2-methylpiperazine-1-carboxylate)

HATU (102 mg, 267 umol, 2.5 eq) was added to a mixture of tert-butyl (2R,5S)-4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-2-methyl-5-(((R)-2-methylpiperazin-1-yl)methyl)piperazine-1-carboxylate (Intermediate 7.2) (150 mg, 235 umol, 2.2 eq), tetradecanedioic acid (27.6 mg, 107 umol, 1 eq), and DIEA (55.2 mg, 427 umol, 74.3 uL, 4 eq) in DCM (2 mL) at 15° C. The mixture was stirred at 35° C. for 12 h after which time LCMS indicated conversion to a product of target mass. The reaction mixture was filtered and concentrated under reduced pressure to give a residue. The residue was purified by preparative HPLC (TFA condition; column: Phenomenex Luna C18 150*30 mm*5 um; mobile phase: [water(0.1% TFA)-MeOH]; B %: 62%-85%, 10 min) to give the title compound (45 mg, 30.0 umol, 28.1% yield) as a white solid.

LCMS (ES, m/z): 750.9 [M/2+H]⁺

### Step 2 of 2: Synthesis of 1,14-bis((R)-4-(((2R,5R)-1-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)tetradecane-1,14-dione

A solution of di-tert-butyl 5,5′-(((2R,2′R)-adipoylbis(2-methylpiperazine-4,1-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate) (45 mg, 30.0 umol, 1 eq) in EtOAc was treated with HCl/EtOAc (4 M, 2 mL) was stirred at 15° C. for 1 h after which time LCMS indicated conversion to a product of target mass. The precipitate was filtered off and the filter cake was dried to give the crude product. The material was purified by preparative HPLC (TFA condition; column: Phenomenex Gemini-NX 150 mm*30 mm*5 um; mobile phase: [water(0.1% TFA)-ACN]; B %: 25%-45%, 12 min) to give the title compound (19.2 mg, 12.3 umol, 41.1% yield, 98% purity, 2 TFA) as a white solid.

LCMS (ES, m/z): 650.6 [M/2+H]⁺

¹H NMR (400 MHz, METHANOL-d₄) δ ppm 8.00-8.13 (m, 2H) 7.24 (dd, J=7.75, 5.75 Hz, 4H) 6.94-7.07 (m, 4H) 3.74-4.02 (m, 14H) 3.55-3.72 (m, 10H) 3.34-3.54 (m, 7H) 3.11-3.29 (m, 7H) 2.59-3.11 (m, 10H) 2.37-2.53 (m, 4H) 1.63 (br t, J=12.76 Hz, 4H) 1.54 (d, J=5.00 Hz, 12H) 1.24-1.39 (m, 27H) 1.20 (br s, 2H)

Examples 32-34 were prepared according to the same procedure as Example 31, replacing tetradecanedioic acid with the appropriate bis-carboxylic acid. The compounds of Examples 32-34 were found to have characterizing data as set forth below.

### Example 32, 1,6-bis((R)-4-(((2R,5R)-1-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)hexane-1,6-dione

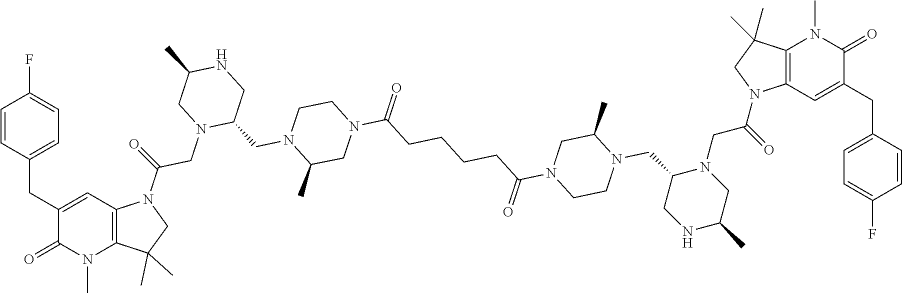 [SMILES: C[C@@H]1CN(CC(=O)N2CC(C)(C)c3c2cc(Cc2ccc(F)cc2)c(=O)n3C)[C@@H](CN2CCN(C(=O)CCCCC(=O)N3CCN(C[C@H]4CN[C@H](C)CN4CC(=O)N4CC(C)(C)c5c4cc(Cc4ccc(F)cc4)c(=O)n5C)[C@H](C)C3)C[C@H]2C)CN1]

LCMS (ES, m/z): 1187.8 [M+H]⁺

¹H NMR (400 MHz, METHANOL-d₄) δ=8.02 (br d, J=4.5 Hz, 2H), 7.26-7.22 (m, 4H), 7.02 (br t, J=8.6 Hz, 4H), 4.04-3.96 (m, 3H), 3.95-3.78 (m, 12H), 3.74-3.64 (m, 10H), 3.61-3.48 (m, 6H), 3.29-3.21 (m, 4H), 3.18-2.93 (m, 11H), 2.67-2.50 (m, 5H), 1.79 (br s, 5H), 1.57-1.53 (m, 12H), 1.34 (br s, 9H), 1.25 (br d, J=4.6 Hz, 3H)

### Example 33, 1,8-bis((R)-4-(((2R,5R)-1-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)octane-1,8-dione

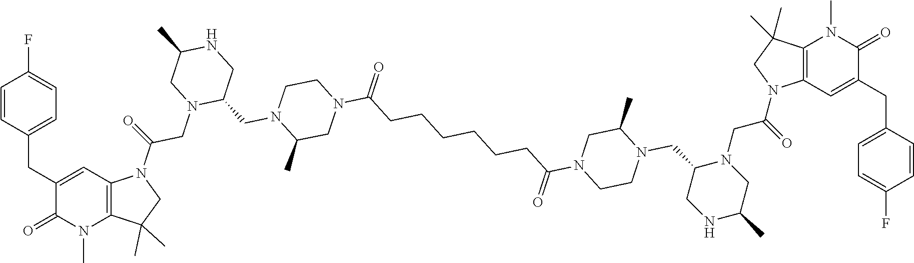 [SMILES: C[C@@H]1CN(CC(=O)N2CC(C)(C)c3c2cc(Cc2ccc(F)cc2)c(=O)n3C)[C@@H](CN2CCN(C(=O)CCCCCCC(=O)N3CCN(C[C@H]4CN[C@H](C)CN4CC(=O)N4CC(C)(C)c5c4cc(Cc4ccc(F)cc4)c(=O)n5C)[C@H](C)C3)C[C@H]2C)CN1]

LCMS (ES, m/z): 1215.9 [M/2+H]P

¹H NMR (400 MHz, DMSO-d₆) δ ppm 10.11 (br s, 2H) 9.36-9.73 (m, 2H) 8.01 (br d, J=3.50 Hz, 2H) 7.26 (dd, J=8.25, 5.75 Hz, 4H) 6.98-7.15 (m, 4H) 3.81-3.97 (m, 11H) 3.58-3.78 (m, 20H) 3.53 (s, 6H)2.85-3.21 (m, 11H) 2.40 (brs, 4H) 1.53 (brs, 4H) 1.43 (s, 12H) 1.16-1.37 (m, 16H).

### Example 34, 1,10-bis((R)-4-(((2R,5R)-1-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)decane-1,10-dione

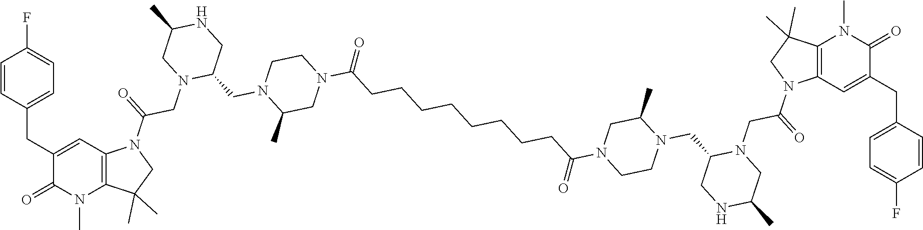 [SMILES: C[C@@H]1CN(CC(=O)N2CC(C)(C)c3c2cc(Cc2ccc(F)cc2)c(=O)n3C)[C@@H](CN2CCN(C(=O)CCCCCCCCC(=O)N3CCN(C[C@H]4CN[C@H](C)CN4CC(=O)N4CC(C)(C)c5c4cc(Cc4ccc(F)cc4)c(=O)n5C)[C@H](C)C3)C[C@H]2C)CN1]

LCMS (ES, m/z): 622.7 [M/2+H]⁺

¹H NMR (400 MHz, METHANOL-d₄) δ ppm 7.89-8.11 (m, 2H) 7.18-7.31 (m, 4H) 7.02 (br t, J=8.50 Hz, 4H) 4.41-4.72 (m, 2H) 4.10-4.33 (m, 2H) 3.77-4.09 (m, 14H) 3.70 (s, 8H) 3.59 (br s, 3H) 3.34-3.49 (m, 4H) 2.84-3.29 (m, 14H) 2.54 (br s, 4H) 1.70 (br s, 4H) 1.57 (d, J=6.50 Hz, 12H) 1.26-1.48 (m, 21H).

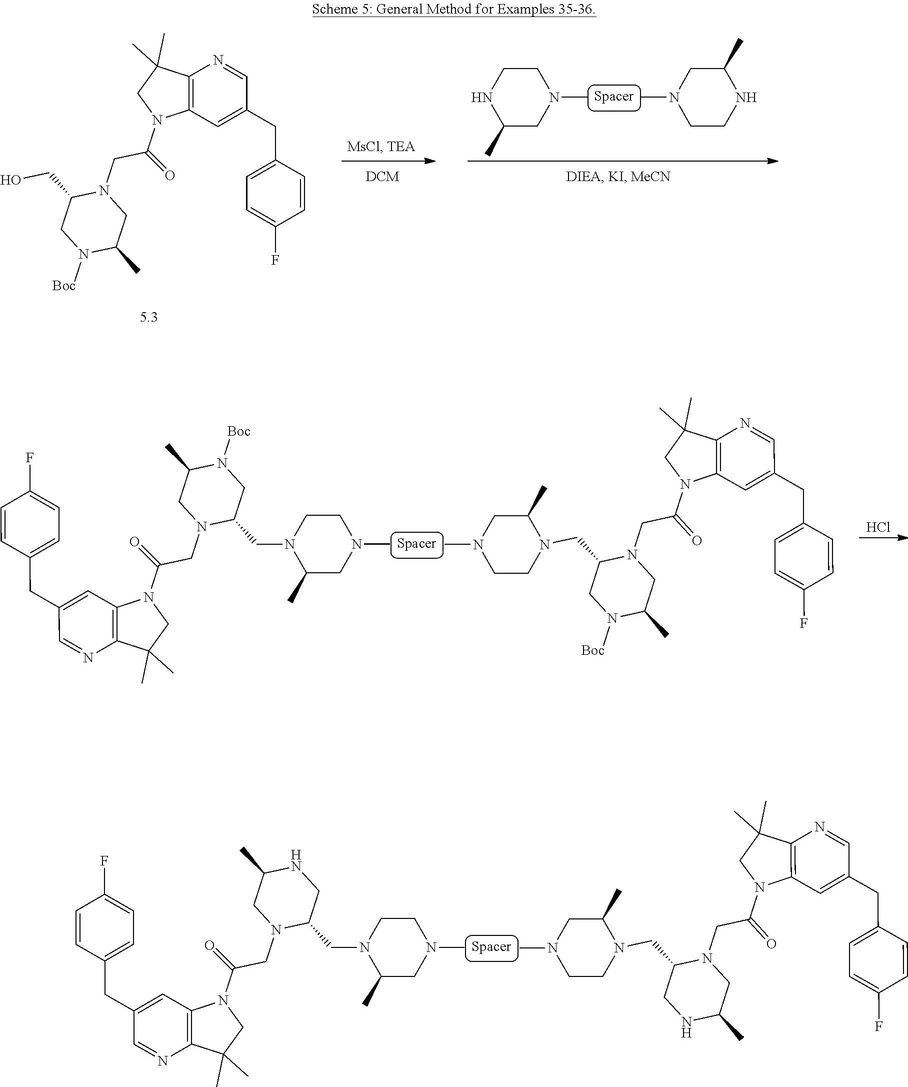

### Example 35, 2,2′-((3R,3′R,6R,6′R)-(((2R,2′R)-isophthaloylbis(2-methylpiperazine-4,1-diyl))bis(methylene))bis(3-methylpiperazine-6,1-diyl))bis(1-(6-(4-fluorobenzyl)-3,3-dimethyl-2,3-dihydro-1H-Pyrrolo[3,2-b]pyridin-1-yl)ethan-1-one)

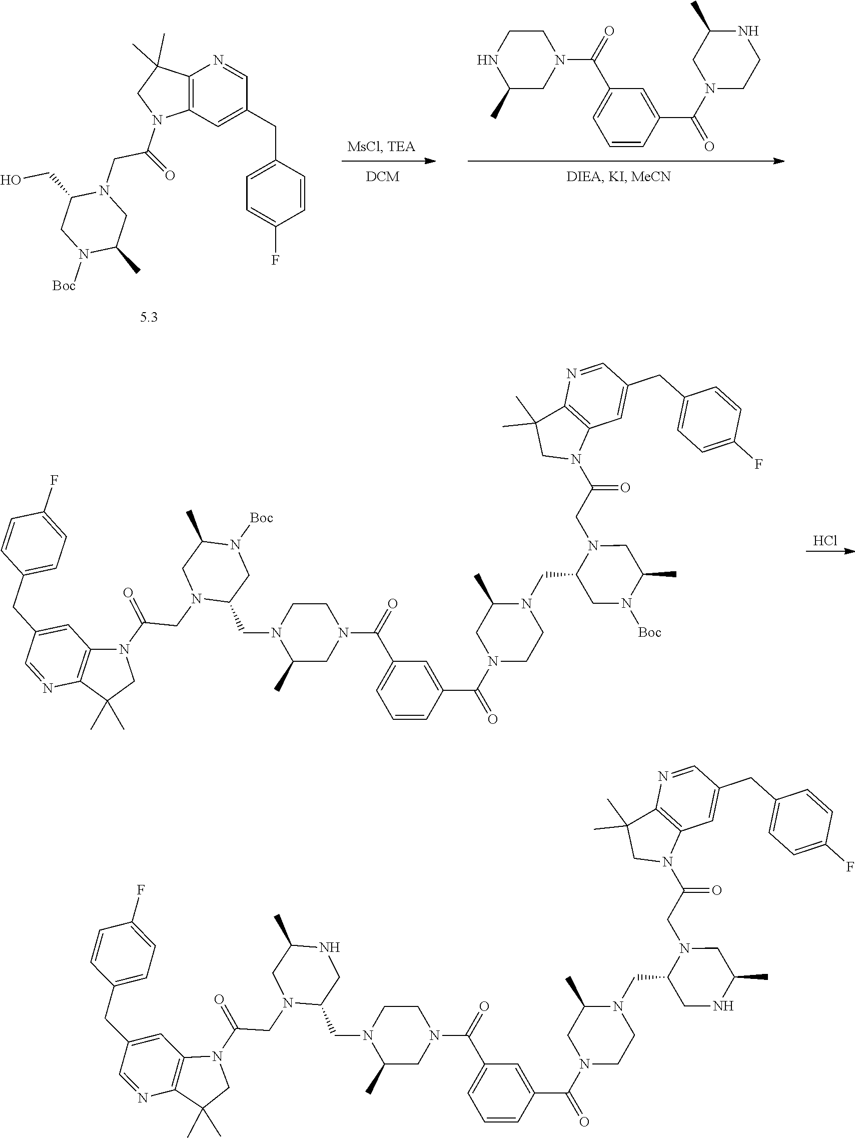

### Step 1 of 2: Synthesis of di-tert-butyl 5,5′-(((2R,2′R)-isophthaloylbis(2-methylpiperazine-4,1-diyl))bis(methylene))(2R,2′R,5S,5'S)-bis(4-(2-(6-(4-fluorobenzyl)-3,3-dimethyl-2,3-dihydro-1H-pyrrolo[3,2-b]pyridin-1-yl)-2-oxoethyl)-2-methylpiperazine-1-carboxylate)

Methylsulfonyl chloride (290 mg, 2.53 mmol) was added to a solution of (2R,5R)-4-{2-[6-(4-fluorobenzyl)-3,3-dimethyl-2,3-dihydro-pyrrolo[3,2-b]pyridin-1-yl]-2-oxo-ethyl}-5-hydroxymethyl-2-methyl-piperazine-1-carboxylic acid tert-butyl ester (760 mg, 1.45 mmol) and triethylamine (293 mg, 2.89 mmol) in dichloromethane (20 mL) at 0° C. The solution was allowed to warm to room temperature and stirred under a nitrogen atmosphere overnight. The mixture was partitioned between aqueous ammonium chloride and dichloromethane and the organic phase was dried over sodium sulphate and evaporated in vacuo. The residue was purified by flash chromatography on silica gel eluting with petroleum/ethyl acetate (1:1) to give the intermediate product (500 mg, 0.917 mmol, 63.7% yield) as a white solid.

To a solution of the intermediate product (150 mg, 0.275 mmol) and 1,3-phenylenebis(((R)-3-methylpiperazin-1-yl)methanone) dihydrochloride (Bis-Amine Intermediate A-017) (55.5 mg, 0.138 mmol) in acetonitrile (20 ml) was added potassium iodide (91.4 mg, 0.550 mmol) and potassium carbonate (152 mg, 1.101 mmol). The resulting mixture was stirred at 70° C. overnight. The reaction mixture was diluted with water (20 mL) and extracted with ethyl acetate (3*20 mL). The organic layers were combined, washed with brine, dried over anhydrous sodium sulfate and concentrated to dryness. The crude product was sequentially purified by preparative TLC (petroleum ether/ethyl acetate=1:3) and then preparative HPLC (column: Phenomenex Gemini C-18 150 mm*21.2 mm*5 um; mobile phase: [water-ACN]; B %: 5%-50%, 10 min) to give the title compound (50 mg, 0.037 mmol, 26.9% Yield) as a colorless oil.

LCMS (ES, m/z): 673.9 [M/2+H]⁺

### Step 2 of 2: Synthesis of 2,2′-((3R,3′R,6R,6′R)-(((2R,2′R)-isophthaloylbis(2-methylpiperazine-4,1-diyl))bis(methylene))bis(3-methylpiperazine-6,1-diyl))bis(1-(6-(4-fluorobenzyl)-3,3-dimethyl-2,3-dihydro-1H-pyrrolo[3,2-b]pyridin-1-yl)ethan-1-one)

To a solution of di-tert-butyl 5,5′-(((2R,2′R)-isophthaloylbis(2-methylpiperazine-4,1-diyl))bis(methylene))(2R,2′R,5S,5'S)-bis(4-(2-(6-(4-fluorobenzyl)-3,3-dimethyl-2,3-dihydro-1H-pyrrolo[3,2-b]pyridin-1-yl)-2-oxoethyl)-2-methylpiperazine-1-carboxylate) (50 mg, 0.037 mmol) in dichloromethane (5 mL) was added 4 M hydrochloric acid in dioxane (5 mL). The resulting mixture was stirred for 4 h at room temperature and then concentrated to dryness to obtain the title compound (10.5 mg, 0.008 mmol, 23.3% yield, 2 HCl) as a white solid.

LCMS (ES, m/z): 1147.5 [M+H]⁺.

¹H NMR (400 MHz, DMSO-d6) δ 10.00 (s, 2H), 9.41 (s, 2H), 8.24 (s, 2H), 8.16 (s, 2H), 7.61 (s, 4H), 7.35-7.25 (t, 4H), 7.15-7.03 (t, 4H), 4.03-3.92 (m, 12H), 3.78-3.52 (m, 12H), 3.49-3.36 (m, 6H), 3.32-3.14 (m, 6H), 3.12-2.81 (m, 6H), 1.35 (s, 12H), 1.24 (s, 12H).

Example 36 was prepared according to the same procedure as Example 35, replacing 1,3-phenylenebis(((R)-3-methylpiperazin-1-yl)methanone) dihydrochloride with 1,3-bis(((R)-3-methylpiperazin-1-yl)sulfonyl)benzene dihydrochloride. The compound of Example 36 was found to have characterizing data as set forth below.

### Example 36, 2,2′-((3R,3′R,6R,6′R)-(((2R,2′R)-(1,3-phenylenedisulfonyl)bis(2-methylpiperazine-4,1-diyl))bis(methylene))bis(3-methylpiperazine-6,1-diyl))bis(1-(6-(4-fluorobenzyl)-3,3-dimethyl-2,3-dihydro-1H-pyrrolo[3,2-b]pyridin-1-yl)ethan-1-one)

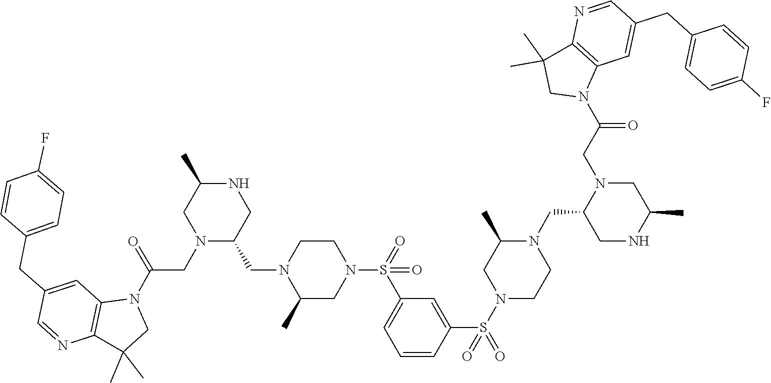 [SMILES: C[C@@H]1CN(CC(=O)N2CC(C)(C)c3ncc(Cc4ccc(F)cc4)cc32)[C@@H](CN2CCN(S(=O)(=O)c3cccc(S(=O)(=O)N4CCN(C[C@H]5CN[C@H](C)CN5CC(=O)N5CC(C)(C)c6ncc(Cc7ccc(F)cc7)cc65)[C@H](C)C4)c3)C[C@H]2C)CN1]

LCMS (ES, m/z): 609.8 [M/2+H]⁺

¹H NMR (400 MHz, DMSO) d ppm δ 10.10 (s, 2H), 9.57 (s, 2H), 8.26 (s, 2H), 8.17 (s, 2H), 8.06 (s, 2H), 8.02 (s, 1H), 7.87 (s, 1H), 7.32 (dd, J=8.4, 5.6, 4H), 7.20-7.12 (m, 4H), 4.12-3.93 (m, 12H), 3.92-3.77 (m, 4H), 3.59-3.36 (m, 12H), 3.32-3.13 (m, 8H), 3.12-2.91 (m, 6H), 1.39-1.30 (m, 12H), 1.27-1.20 (in, 12H).

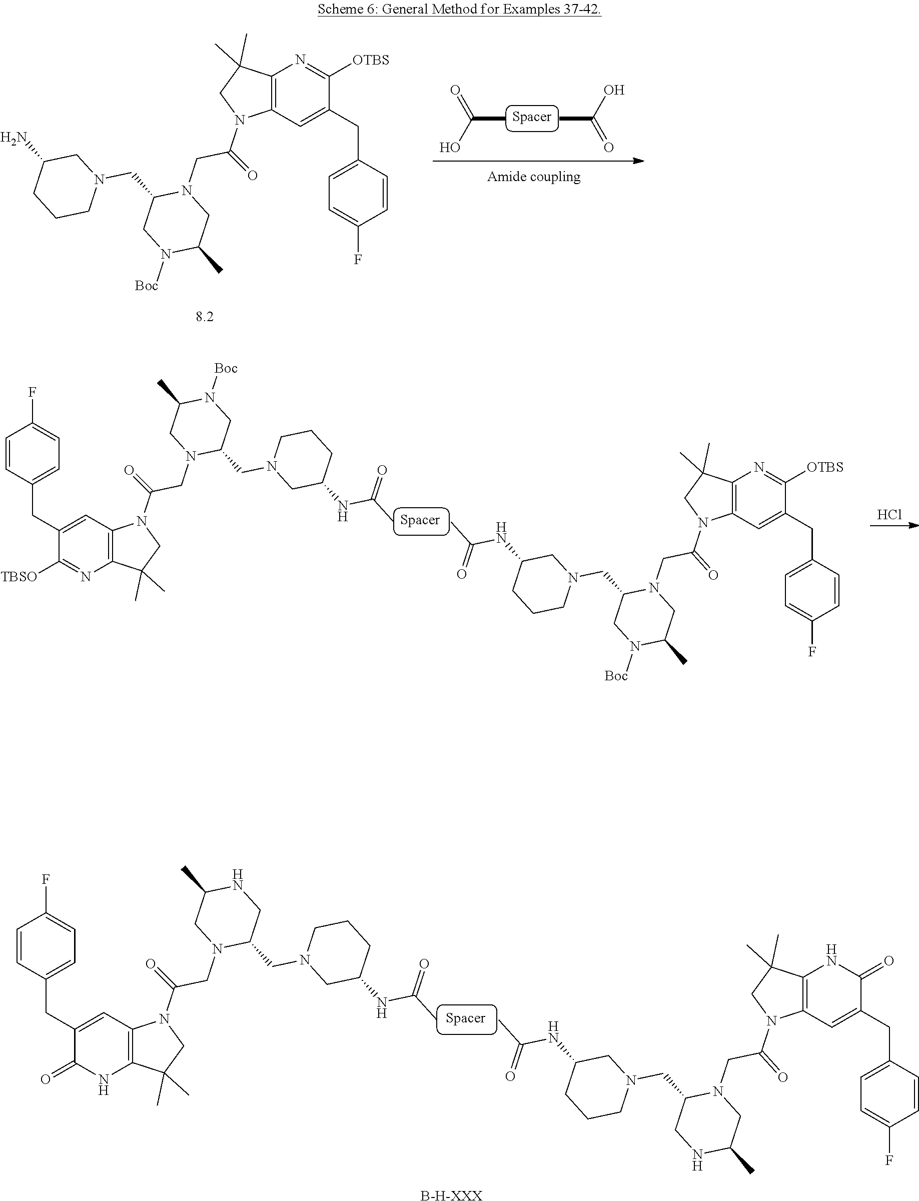

### Example 37, N1,N6-bis((S)-1-(((2R,5R)-1-(2-(6-(4-fluorobenzyl)-3,3-dimethyl-5-oxo-2,3,4,5-tetrahydro-1H-Pyrrolo [3,2-b]Pyridin-1-yl)-2-oxoethyl)-5-methylpiperazin-2-yl)methyl)piperidin-3-yl)adipamide

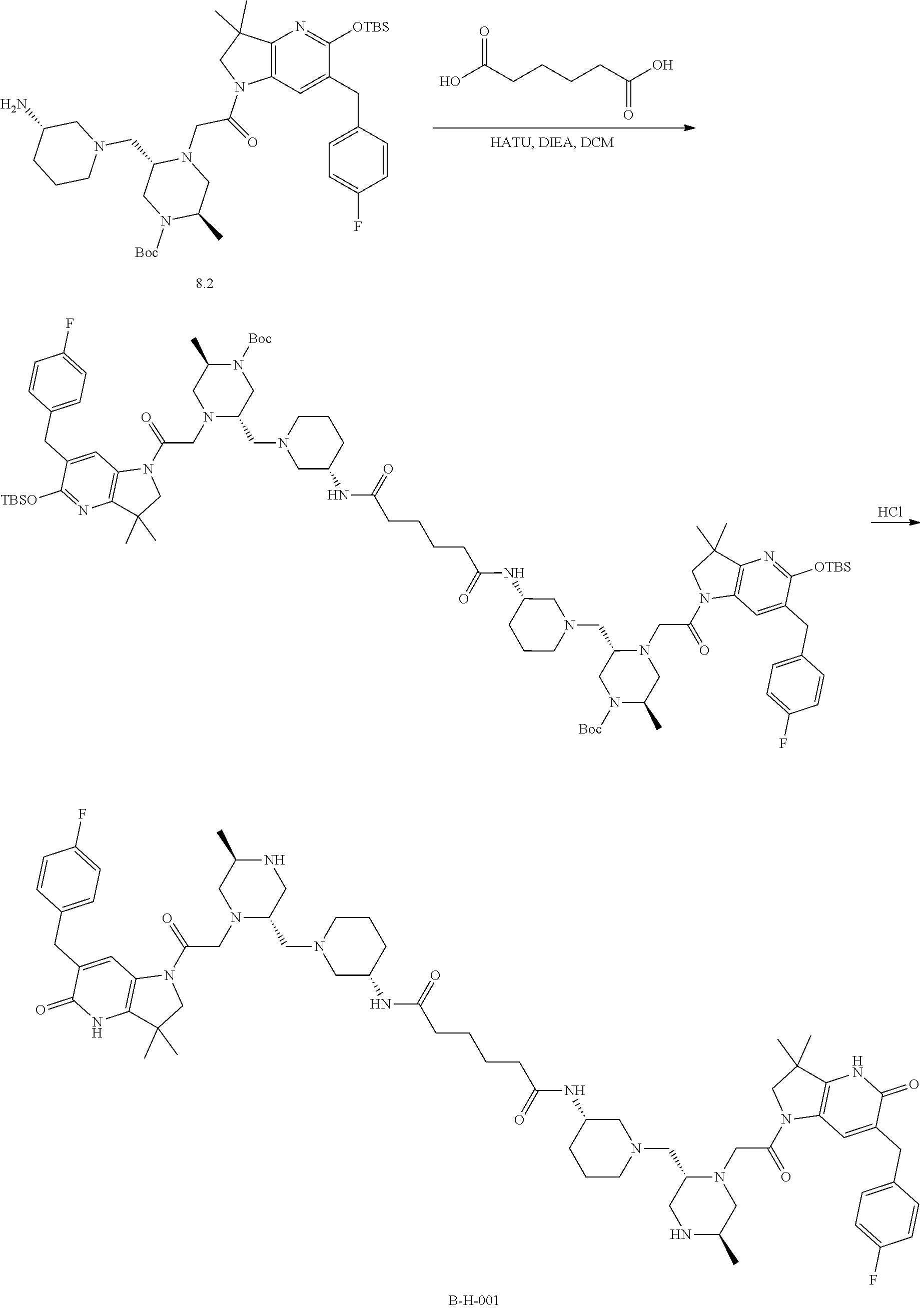

### Step 1 of 2: Synthesis of di-tert-butyl 5,5′-(((3S,3'S)-(adipoylbis(azanediyl))bis(piperidine-3,1-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-Pyrrolo[3,2-b]Pyridin-1-yl)-2-oxoethyl)-2-methylpiperazine-1-carboxylate)

To a solution of tert-butyl (2R,5S)-5-(((S)-3-aminopiperidin-1l-yl)methyl)-4-(2-(5-((tert-butyldimethylsilyl)oxy)-6-(4-fluorobenzyl)-3, 3-dimethyl-2,3-dihydro-1H-pyrrolo[3,2-b]pyridin-1-yl)-2-oxoethyl)-2-methylpiperazine-1-carboxylate (150 mg, 203 umol, 2.2 eq), adipic acid (13.5 mg, 92.3 umol, 15.3 uL, 1 eq) in DCM (2 mL) was added DIEA (47.7 mg, 369 umol, 64.3 uL, 4 eq) and HATU (87.7 mg, 231 umol, 2.5 eq). The mixture was stirred at 30° C. for 12 h after which time LCMS indicated conversion to product of target mass. The reaction was concentrated in vacuo and the residue was purified by preparative TLC (SiO₂, Ethyl acetate/Methanol=10:1) to give the title compound (90 mg, 56.7 umol, 61.4% yield) as a yellow oil.

TLC (Ethyl acetate/Methanol=10:1) R_{f}=0.43

LCMS (ES, m/z): 1589.0 [M+H]⁺

### Step 2 of 2: Synthesis of N1,N6-bis((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)adipamide

To a solution of di-tert-butyl 5,5′-(((3S,3'S)-(adipoylbis(azanediyl))bis(piperidine-3,1-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate) (90 mg, 56.7 umol, 1 eq) in EtOAc (1 mL) was added HCl/EtOAc (4 M, 2 mL, 141 eq). The mixture was stirred at 15° C. for 1 h after which time LCMS indicated complete conversion to a product of target mass. The reaction mixture was filtered to give the crude product. The crude material was purified by preparative HPLC (TFA condition; column: Phenomenex Luna C18 100*30 mm*5 um; mobile phase: [water(0.1% TFA)-ACN]; B %: 10%-30%, 10 min) to give the title compound (24.1 mg, 17.3 umol, 30.4% yield, 99.3% purity, 2 TFA) as a white solid.

LCMS (ES, m/z): 580.6 [M/2+H]⁺

¹H NMR (400 MHz, METHANOL-d₄) δ=8.15 (s, 2H), 7.23 (dd, J=5.5, 8.6 Hz, 4H), 6.98 (t, J=8.8 Hz, 4H), 4.01 (br d, J=11.0 Hz, 3H), 3.89 (br d, J=10.5 Hz, 3H), 3.85-3.76 (m, 7H), 3.70 (br s, 3H), 3.52 (br s, 3H), 3.31 (s, 5H), 3.29-3.05 (m, 10H), 3.01 (br d, J=11.4 Hz, 2H), 2.74 (br s, 3H), 2.28-1.94 (m, 8H), 1.75 (br s, 2H), 1.57 (br s, 5H), 1.42 (d, J=4.4 Hz, 12H), 1.29 (br d, J=6.6 Hz, 6H).

Examples 38-42 were prepared according to the same procedure as Example 37, replacing adipic acid with the appropriate bis-carboxylic acid. The compounds of Examples 38-42 were found to have characterizing data as set forth below.

### Example 38, N¹,N⁸-bis((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)octanediamide

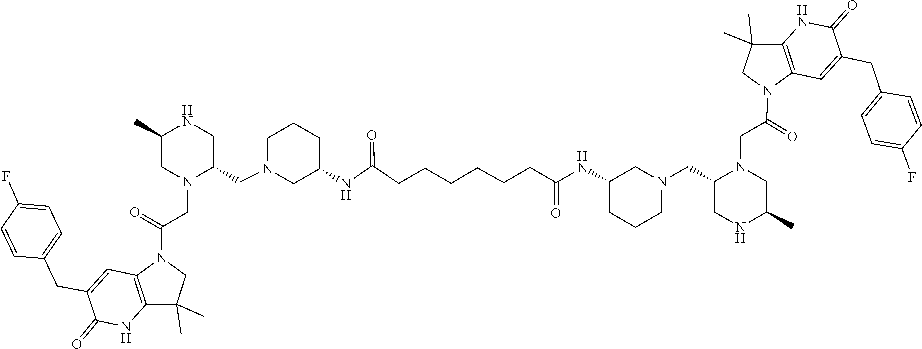 [SMILES: C[C@@H]1CN(CC(=O)N2CC(C)(C)c3[nH]c(=O)c(Cc4ccc(F)cc4)cc32)[C@@H](CN2CCC[C@H](NC(=O)CCCCCCC(=O)N[C@H]3CCCN(C[C@H]4CN[C@H](C)CN4CC(=O)N4CC(C)(C)c5[nH]c(=O)c(Cc6ccc(F)cc6)cc54)C3)C2)CN1]

LCMS (ES, m/z): 594.6 [M/2+H]⁺

¹H NMR (400 MHz, METHANOL-d4) δ=8.16 (s, 2H), 7.24 (dd, J=5.5, 8.6 Hz, 4H), 7.04-6.94 (m, 4H), 4.05-3.96 (m, 3H), 3.93-3.85 (m, 3H), 3.83 (br s, 3H), 3.78 (s, 4H), 3.67 (br s, 2H), 3.48 (br d, J=3.1 Hz, 2H), 3.30-3.29 (m, 10H), 3.26-2.97 (m, 15H), 2.19-1.94 (m, 8H), 1.53 (br s, 4H), 1.42 (d, J=4.8 Hz, 12H), 1.29 (br d, J=6.1 Hz, 10H).

### Example 39, N¹,N¹⁰-bis((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)decanediamide

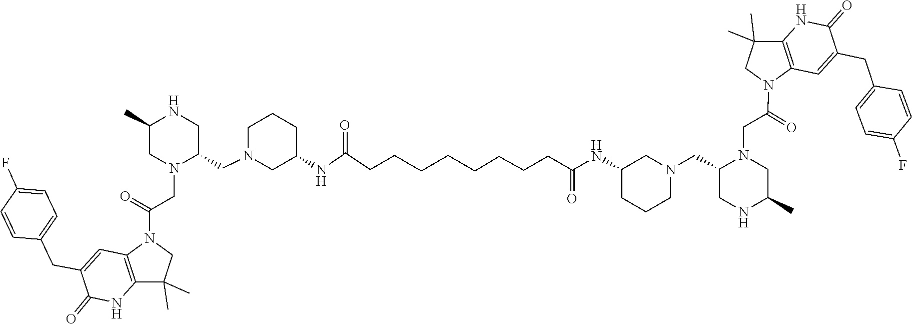 [SMILES: C[C@@H]1CN(CC(=O)N2CC(C)(C)c3[nH]c(=O)c(Cc4ccc(F)cc4)cc32)[C@@H](CN2CCC[C@H](NC(=O)CCCCCCCCC(=O)N[C@H]3CCCN(C[C@H]4CN[C@H](C)CN4CC(=O)N4CC(C)(C)c5[nH]c(=O)c(Cc6ccc(F)cc6)cc54)C3)C2)CN1]

LCMS (ES, m/z): 608.6 [M/2+H]⁺

¹H NMR (400 MHz, METHANOL-d4) δ=8.18 (s, 2H), 7.25 (dd, J=5.5, 8.6 Hz, 4H), 6.99 (t, J=8.8 Hz, 4H), 4.03 (d, J=11.0 Hz, 3H), 3.91 (br d, J=10.5 Hz, 2H), 3.87-3.77 (m, 8H), 3.76-3.63 (m, 3H), 3.53 (br s, 2H), 3.43 (br s, 1H), 3.28-3.10 (m, 11H), 3.02 (br d, J=14.0 Hz, 2H), 2.78 (br s, 4H), 2.23-1.94 (m, 8H), 1.77 (br s, 2H), 1.54 (br s, 5H), 1.42 (s, 12H), 1.40-1.33 (m, 3H), 1.30 (br d, J=6.1 Hz, 6H), 1.23 (br s, 8H).

### Example 40, N¹,N¹⁴-bis((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)tetradecanediamide

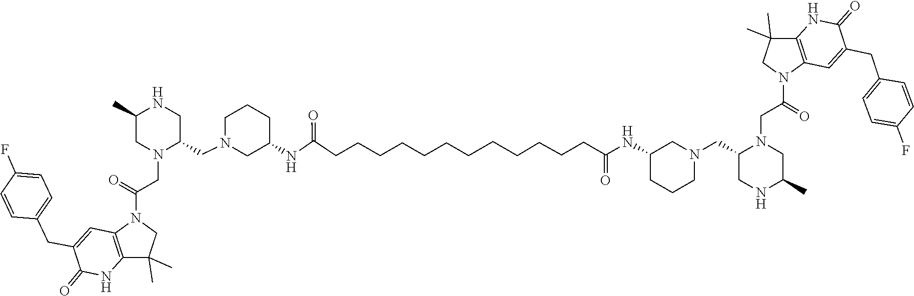 [SMILES: C[C@@H]1CN(CC(=O)N2CC(C)(C)c3[nH]c(=O)c(Cc4ccc(F)cc4)cc32)[C@@H](CN2CCC[C@H](NC(=O)CCCCCCCCCCCCC(=O)N[C@H]3CCCN(C[C@H]4CN[C@H](C)CN4CC(=O)N4CC(C)(C)c5[nH]c(=O)c(Cc6ccc(F)cc6)cc54)C3)C2)CN1]

LCMS (ES, m/z): 636.7 [M/2+H]

¹H NMR (400 MHz, METHANOL-d4) δ=8.20 (br s, 2H), 7.32-7.21 (m, 4H), 6.99 (t, J=8.8 Hz, 4H), 4.03 (br d, J=10.5 Hz, 4H), 3.90 (br d, J=10.5 Hz, 3H), 3.86 (br s, 4H), 3.78 (s, 4H), 3.69 (br s, 3H), 3.58-3.32 (m, 4H), 3.29-3.07 (m, 13H), 3.00 (br s, 2H), 2.88-2.54 (m, 3H), 2.16 (br s, 8H), 1.80 (br s, 2H), 1.56 (br s, 5H), 1.45-1.39 (m, 12H), 1.29 (br d, J=6.6 Hz, 6H), 1.22 (br d, J=17.5 Hz, 15H).

### Example 41, 2,2′-(1,3-phenylene)bis(N-((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)acetamide)

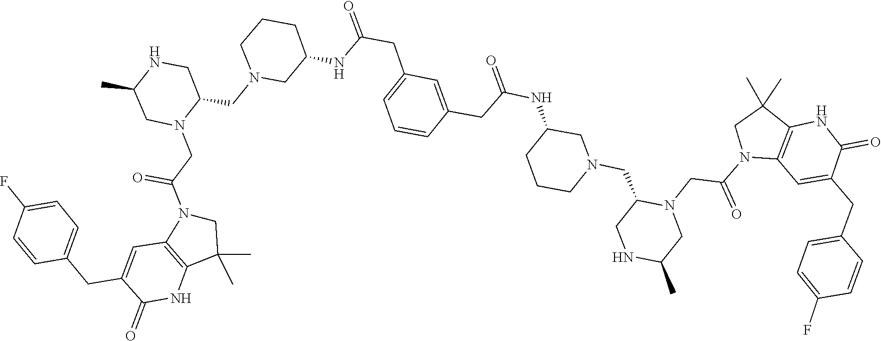 [SMILES: C[C@@H]1CN(CC(=O)N2CC(C)(C)c3[nH]c(=O)c(Cc4ccc(F)cc4)cc32)[C@@H](CN2CCC[C@H](NC(=O)Cc3cccc(CC(=O)N[C@H]4CCCN(C[C@H]5CN[C@H](C)CN5CC(=O)N5CC(C)(C)c6[nH]c(=O)c(Cc7ccc(F)cc7)cc65)C4)c3)C2)CN1]

LCMS (ES, m/z): 604.6 [M/2+H]⁺

¹H NMR (400 MHz, METHANOL-d4) δ=8.07 (br s, 2H), 7.29-7.13 (m, 4H), 7.06 (br s, 4H), 6.88 (br s, 4H), 4.01 (br d, J=11.0 Hz, 2H), 3.87 (br d, J=10.8 Hz, 4H), 3.81-3.72 (m, 7H), 3.68 (br s, 5H), 3.55-3.36 (m, 9H), 3.26-3.07 (m, 12H), 3.04-2.64 (m, 3H), 2.18-1.91 (m, 5H), 1.82-1.46 (m, 5H), 1.41 (d, J=6.4 Hz, 12H), 1.27 (br d, J=5.5 Hz, 6H).

### Example 42, 2,2′-(1,4-phenylene)bis(N-((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)acetamide)

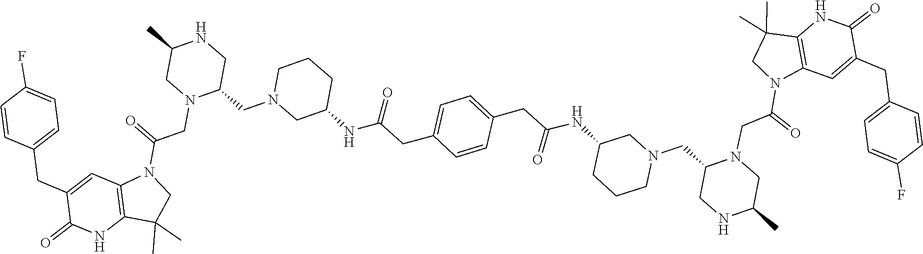 [SMILES: C[C@@H]1CN(CC(=O)N2CC(C)(C)c3[nH]c(=O)c(Cc4ccc(F)cc4)cc32)[C@@H](CN2CCC[C@H](NC(=O)Cc3ccc(CC(=O)N[C@H]4CCCN(C[C@H]5CN[C@H](C)CN5CC(=O)N5CC(C)(C)c6[nH]c(=O)c(Cc7ccc(F)cc7)cc65)C4)cc3)C2)CN1]

LCMS (ES, m/z): 604.6 [M/2+H]⁺

¹H NMR (400 MHz, METHANOL-d4) δ=8.18-7.97 (m, 2H), 7.24 (br s, 4H), 7.05 (br s, 4H), 6.87 (br s, 4H), 4.02 (br d, J=10.6 Hz, 2H), 3.87 (br d, J=11.0 Hz, 4H), 3.80-3.73 (m, 7H), 3.67 (br s, 5H), 3.54-3.37 (m, 9H), 3.16 (br d, J=19.6 Hz, 12H), 3.03-2.58 (m, 3H), 2.17-1.90 (m, 5H), 1.81-1.47 (m, 5H), 1.42 (d, J=5.4 Hz, 12H), 1.28 (br d, J=5.5 Hz, 6H).

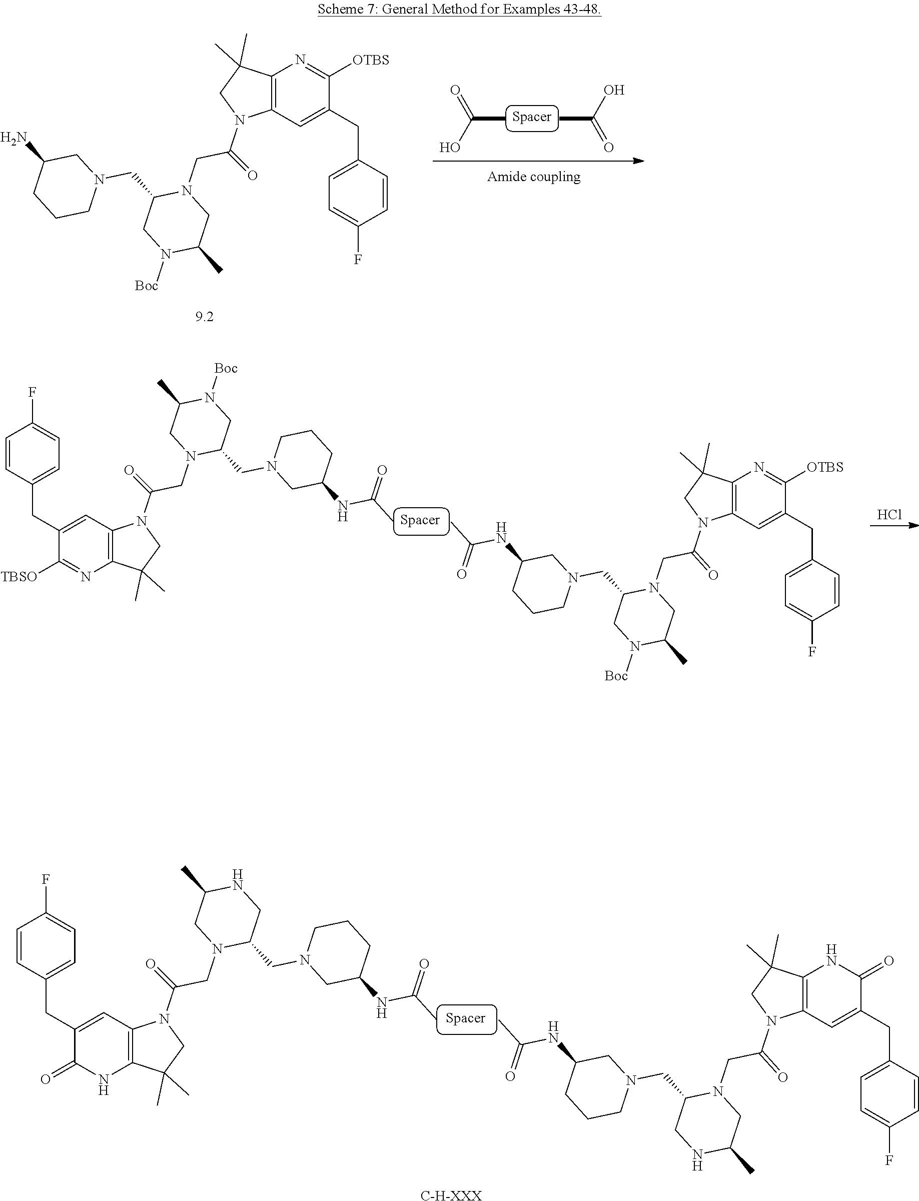

### Example 43, N1,N6-bis((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)adipamide

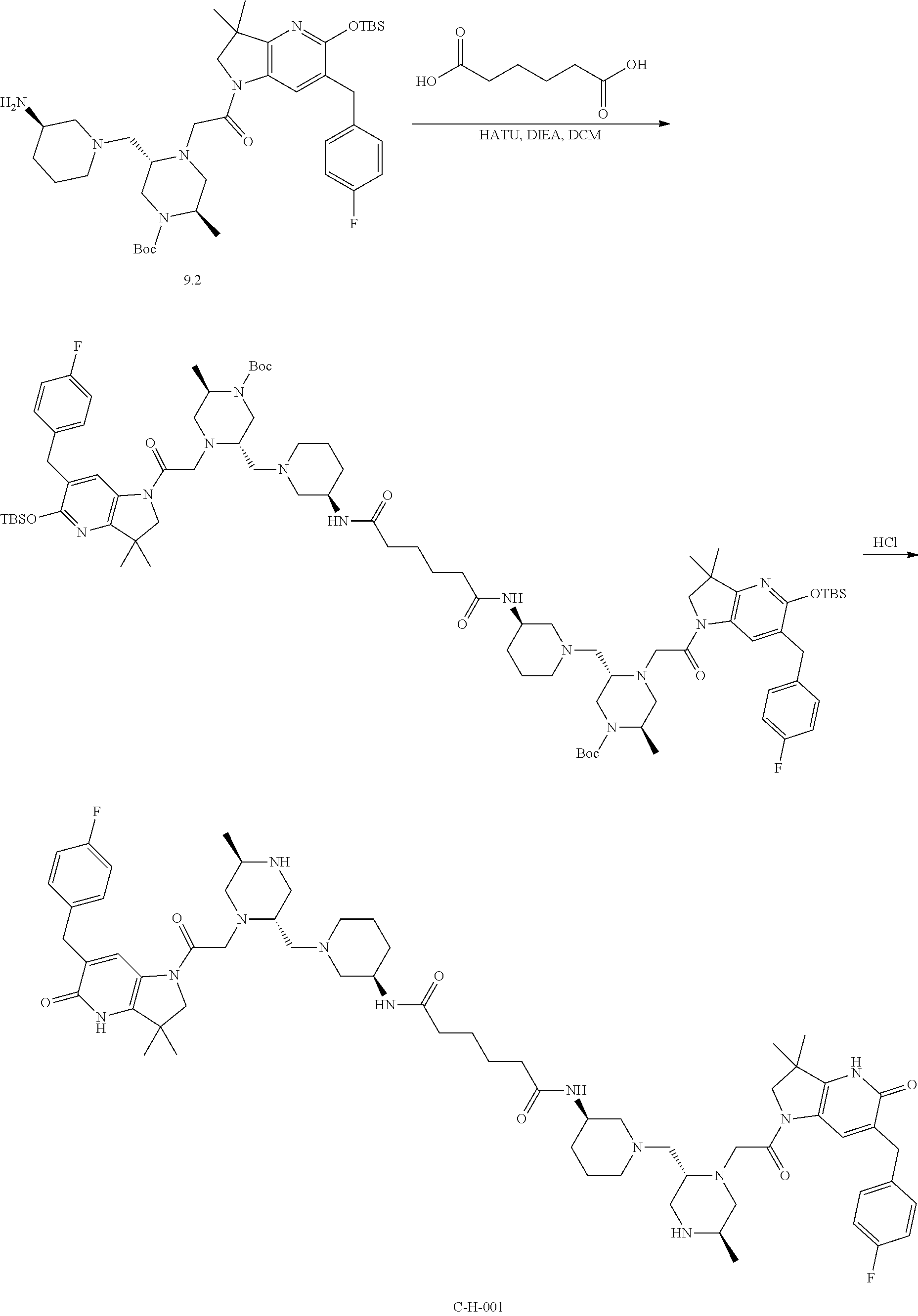

### Step 1 of 2: Synthesis of di-tert-butyl 5,5′-(((3R,3′R)-(adipoylbis(azanediyl))bis(piperidine-3,1-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b-]pyridin-1-yl)-2-oxoethyl)-2-methylpiperazine-1-carboxylate)

To a solution of tert-butyl (2R,5S)-5-(((R)-3-aminopiperidin-1-yl)methyl)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate (120 mg, 162 umol, 2.3 eq), adipic acid (10.3 mg, 70.6 umol, 11.7 uL, 1 eq) in DCM (3 mL) was added DIEA (36.5 mg, 282 umol, 49.2 uL, 4 eq) and HATU (67.1 mg, 177 umol, 2.5 eq). The mixture was stirred at 15° C. for 2 h after which time LCMS indicated conversion to product of target mass. The reaction was concentrated in vacuo and the residue was purified by preparative TLC (SiO₂, Ethyl acetate/Methanol=10:1) to give the title compound (110 mg, 69.2 umol, 98% yield) as a yellow oil.

LCMS (ES, m/z): 1588.6 [M+H]⁺

### Step 2 of 2: Synthesis of N1,N6-bis((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)adipamide

To a solution of di-tert-butyl 5,5′-(((3R,3′R)-(adipoylbis(azanediyl))bis(piperidine-3,1-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate) (110 mg, 69.2 umol, 1 eq) in EtOAc (1.5 mL) was added HCl/EtOAc (4 M, 1.5 mL). The mixture was stirred at 15° C. for 2 h after which time LCMS indicated complete conversion to a product of target mass. The reaction mixture was filtered to give the crude product. The crude material was purified by preparative HPLC (FA condition; column: Phenomenex Luna C18 100*30 mm*5 um; mobile phase: [water(0.2% FA)-ACN]; B %: 5%-35%, 9 min) to give the title compound (9.4 mg, 7.37 umol, 10.64% yield, 98% purity, 2 FA) as a white solid.

LCMS (ES, m/z): 580.6 [M/2+H]⁺

¹H NMR (400 MHz, METHANOL-d4) δ=8.46 (s, 2H), 7.23 (br t, J=6.4 Hz, 4H), 7.03-6.93 (m, 4H), 3.96-3.62 (m, 13H), 3.50 (br d, J=17.1 Hz, 2H), 3.22 (br s, 3H), 3.29-3.11 (m, 1H), 3.06-2.93 (m, 4H), 2.89-2.60 (m, 9H), 2.13 (br s, 6H), 1.99-1.84 (m, 2H), 1.80-1.47 (m, 10H), 1.40 (br d, J=4.4 Hz, 12H), 1.37-1.32 (m, 2H), 1.25 (br d, J=6.1 Hz, 6H), 1.20-1.12 (m, 2H).

Examples 44-48 were prepared according to the same procedure as Example 43, replacing adipic acid with the appropriate bis-carboxylic acid. The compounds of Examples 44-48 were found to have characterizing data as set forth below.

### Example 44, N¹,N⁸-bis((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)octanediamide

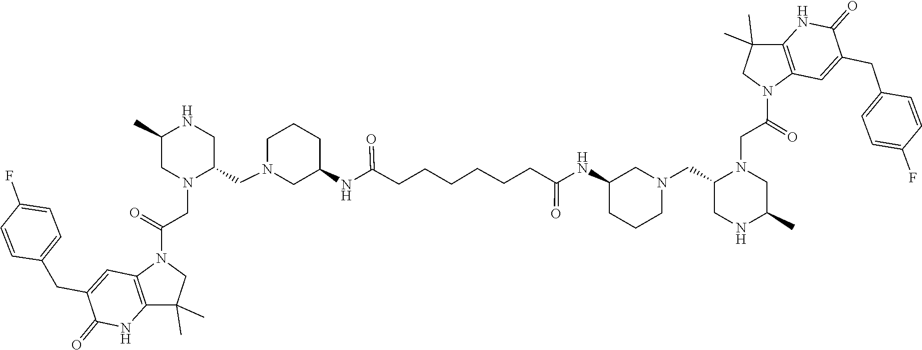 [SMILES: C[C@@H]1CN(CC(=O)N2CC(C)(C)c3[nH]c(=O)c(Cc4ccc(F)cc4)cc32)[C@@H](CN2CCC[C@@H](NC(=O)CCCCCCC(=O)N[C@@H]3CCCN(C[C@H]4CN[C@H](C)CN4CC(=O)N4CC(C)(C)c5[nH]c(=O)c(Cc6ccc(F)cc6)cc54)C3)C2)CN1]

LCMS (ES, m/z): 594.6 [M/2+H]⁺

¹H NMR (400 MHz, METHANOL-d4) δ=8.41 (s, 2H), 7.23 (br t, J=5.9 Hz, 4H), 7.03-6.93 (m, 4H), 3.97-3.57 (m, 13H), 3.51 (br d, J=17.1 Hz, 2H), 3.23 (br s, 2H), 3.01 (br d, J=10.5 Hz, 4H), 2.88-2.60 (m, 9H), 2.23-2.01 (m, 6H), 1.92 (br s, 2H), 1.82-1.44 (m, 10H), 1.40 (br d, J=3.9 Hz, 12H), 1.29 (br s, 4H), 1.26 (br d, J=5.7 Hz, 7H), 1.16 (br d, J=11.0 Hz, 3H).

### Example 45, N1,N¹⁰-bis((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)decanediamide

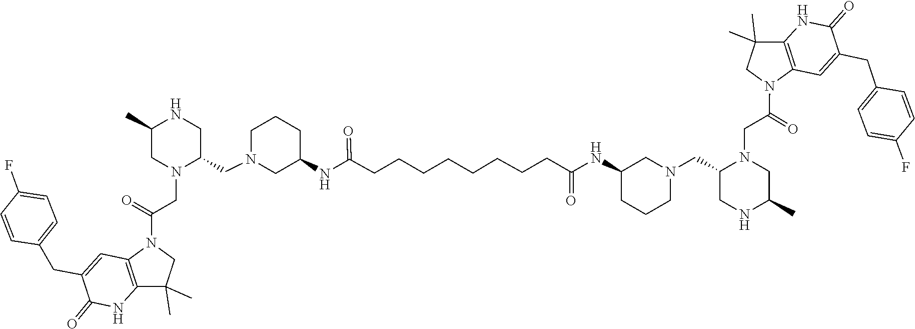 [SMILES: C[C@@H]1CN(CC(=O)N2CC(C)(C)c3[nH]c(=O)c(Cc4ccc(F)cc4)cc32)[C@@H](CN2CCC[C@@H](NC(=O)CCCCCCCCC(=O)N[C@@H]3CCCN(C[C@H]4CN[C@H](C)CN4CC(=O)N4CC(C)(C)c5[nH]c(=O)c(Cc6ccc(F)cc6)cc54)C3)C2)CN1]

LCMS (ES, m/z): 608.6 [M/2+H]⁺

¹H NMR (400 MHz, METHANOL-d4) δ=8.12 (br s, 2H), 7.25 (br s, 4H), 7.02 (br t, J=8.1 Hz, 4H), 4.11-3.95 (m, 4H), 3.89 (br d, J=10.1 Hz, 3H), 3.82 (br d, J=15.8 Hz, 6H), 3.76 (br s, 1H), 3.67 (br s, 3H), 3.52 (br s, 2H), 3.31-3.04 (m, 17H), 3.03-2.89 (m, 4H), 2.62 (br s, 1H), 2.19 (br s, 4H), 2.08-1.87 (m, 4H), 1.58 (br s, 7H), 1.42 (br d, J=9.2 Hz, 12H), 1.33 (br d, J=6.1 Hz, 6H), 1.29 (br s, 6H).

### Example 46, N1,N¹⁴-bis((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)tetradecanediamide

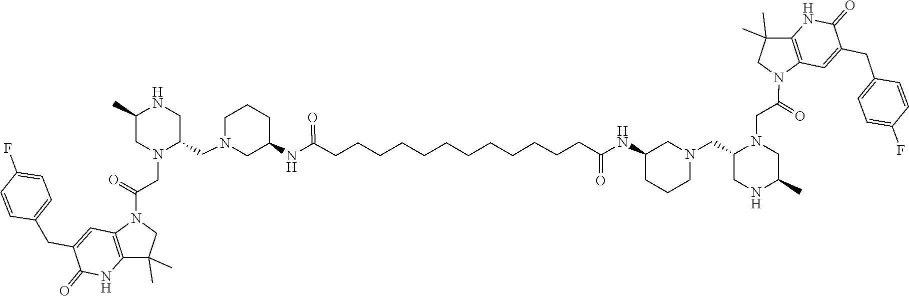 [SMILES: C[C@@H]1CN(CC(=O)N2CC(C)(C)c3[nH]c(=O)c(Cc4ccc(F)cc4)cc32)[C@@H](CN2CCC[C@@H](NC(=O)CCCCCCCCCCCCC(=O)N[C@@H]3CCCN(C[C@H]4CN[C@H](C)CN4CC(=O)N4CC(C)(C)c5[nH]c(=O)c(Cc6ccc(F)cc6)cc54)C3)C2)CN1]

LCMS (ES, m/z): 636.7 [M/2+H]⁺

¹H NMR (400 MHz, METHANOL-d4) δ=8.48 (s, 2H), 8.28 (s, 2H), 7.23 (br t, J=5.9 Hz, 4H), 7.03-6.93 (m, 4H), 3.95-3.68 (m, 13H), 3.49 (br d, J=17.5 Hz, 2H), 3.28 (br s, 2H), 3.20 (br s, 2H), 3.01 (br d, J=12.3 Hz, 4H), 2.90-2.73 (m, 5H), 2.67 (br d, J=8.3 Hz, 4H), 2.21-2.06 (m, 6H), 1.93 (br t, J=10.3 Hz, 2H), 1.81-1.47 (m, 10H), 1.40 (br d, J=3.1 Hz, 12H), 1.27 (br s, 19H), 1.25 (br s, 4H), 1.16 (br d, J=11.4 Hz, 3H).

### Example 47, 2,2′-(1,3-phenylene)bis(N-((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)acetamide)

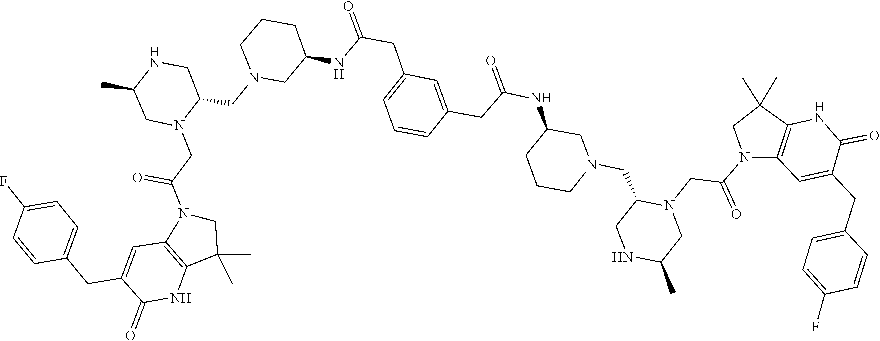 [SMILES: C[C@@H]1CN(CC(=O)N2CC(C)(C)c3[nH]c(=O)c(Cc4ccc(F)cc4)cc32)[C@@H](CN2CCC[C@@H](NC(=O)Cc3cccc(CC(=O)N[C@@H]4CCCN(C[C@H]5CN[C@H](C)CN5CC(=O)N5CC(C)(C)c6[nH]c(=O)c(Cc7ccc(F)cc7)cc65)C4)c3)C2)CN1]

LCMS (ES, m/z): 604.6 [M/2+H]⁺

¹H NMR (400 MHz, METHANOL-d4) δ=8.42 (s, 2H), 8.25 (s, 2H), 7.24-7.11 (m, 8H), 6.96 (br t, J=8.1 Hz, 4H), 3.95-3.61 (m, 14H), 3.51 (br d, J=16.2 Hz, 2H), 3.42 (s, 4H), 3.26 (br d, J=13.6 Hz, 6H), 3.06-2.91 (m, 4H), 2.90-2.50 (m, 10H), 2.14 (br d, J=11.4 Hz, 2H), 1.95 (br s, 2H), 1.74 (br s, 2H), 1.62 (br d, J=12.7 Hz, 2H), 1.38 (br d, J=4.8 Hz, 12H), 1.24 (br d, J=5.7 Hz, 6H), 1.19 (br d, J=11.4 Hz, 2H).

### Example 48, 2,2′-(1,4-phenylene)bis(N-((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)acetamide)

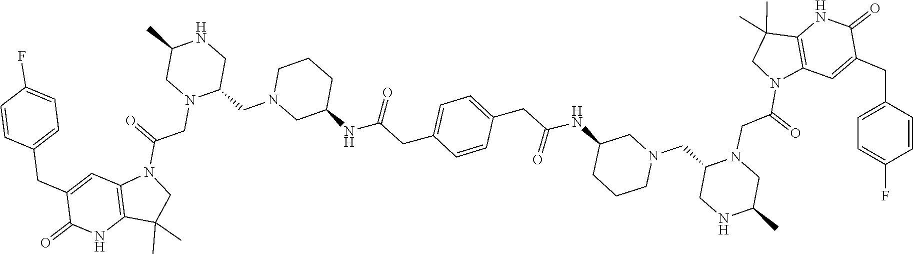 [SMILES: C[C@@H]1CN(CC(=O)N2CC(C)(C)c3[nH]c(=O)c(Cc4ccc(F)cc4)cc32)[C@@H](CN2CCC[C@@H](NC(=O)Cc3ccc(CC(=O)N[C@@H]4CCCN(C[C@H]5CN[C@H](C)CN5CC(=O)N5CC(C)(C)c6[nH]c(=O)c(Cc7ccc(F)cc7)cc65)C4)cc3)C2)CN1]

LCMS (ES, m/z): 604.5 [M/2+H]⁺

¹H NMR (400 MHz, METHANOL-d4) δ=8.09 (s, 2H), 7.24-7.13 (m, 8H), 7.00-6.92 (m, 4H), 4.07-3.94 (m, 4H), 3.91-3.76 (m, 8H), 3.75-3.57 (m, 7H), 3.56-3.41 (m, 7H), 3.40-3.33 (m, 2H), 3.28 (br s, 4H), 3.21-2.86 (m, 7H), 2.78-2.46 (m, 2H), 2.08-1.88 (m, 4H), 1.59 (br s, 4H), 1.40 (br d, J=4.8 Hz, 13H), 1.33 (br d, J=6.1 Hz, 6H).

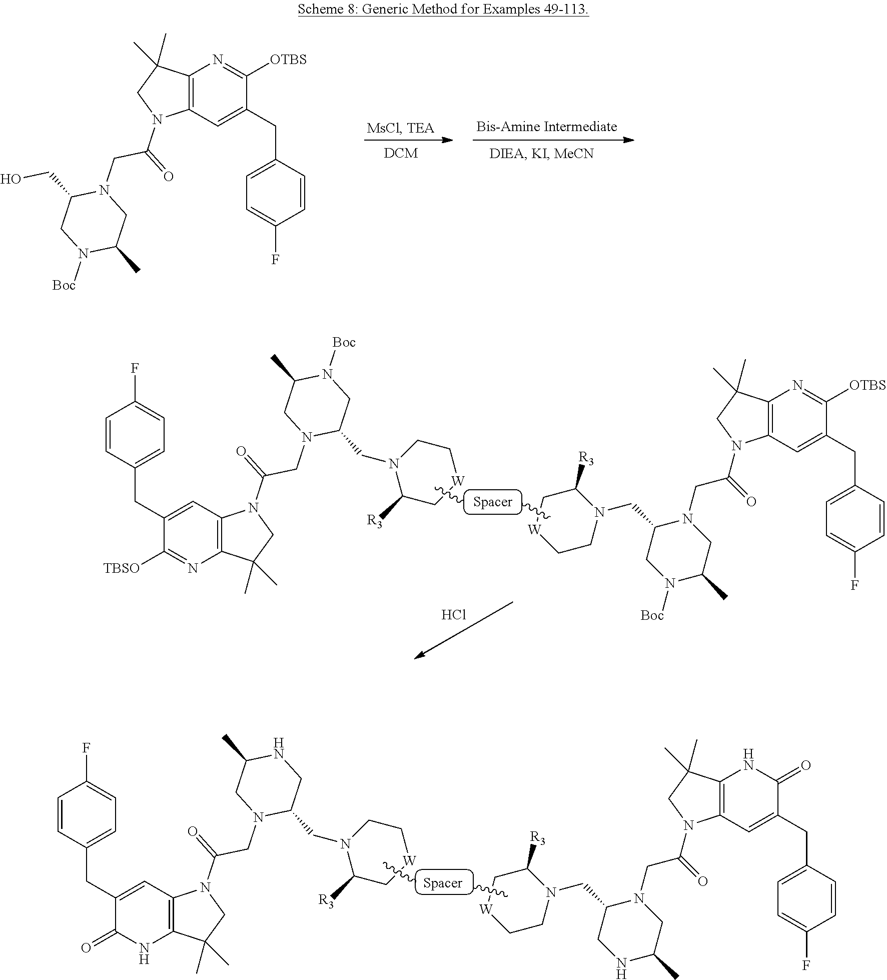

### Example 49, 1,1′-(2,2′-((3R,3′R,6R,6′R)-(((2S,2'S)-(((1,2-phenylenebis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

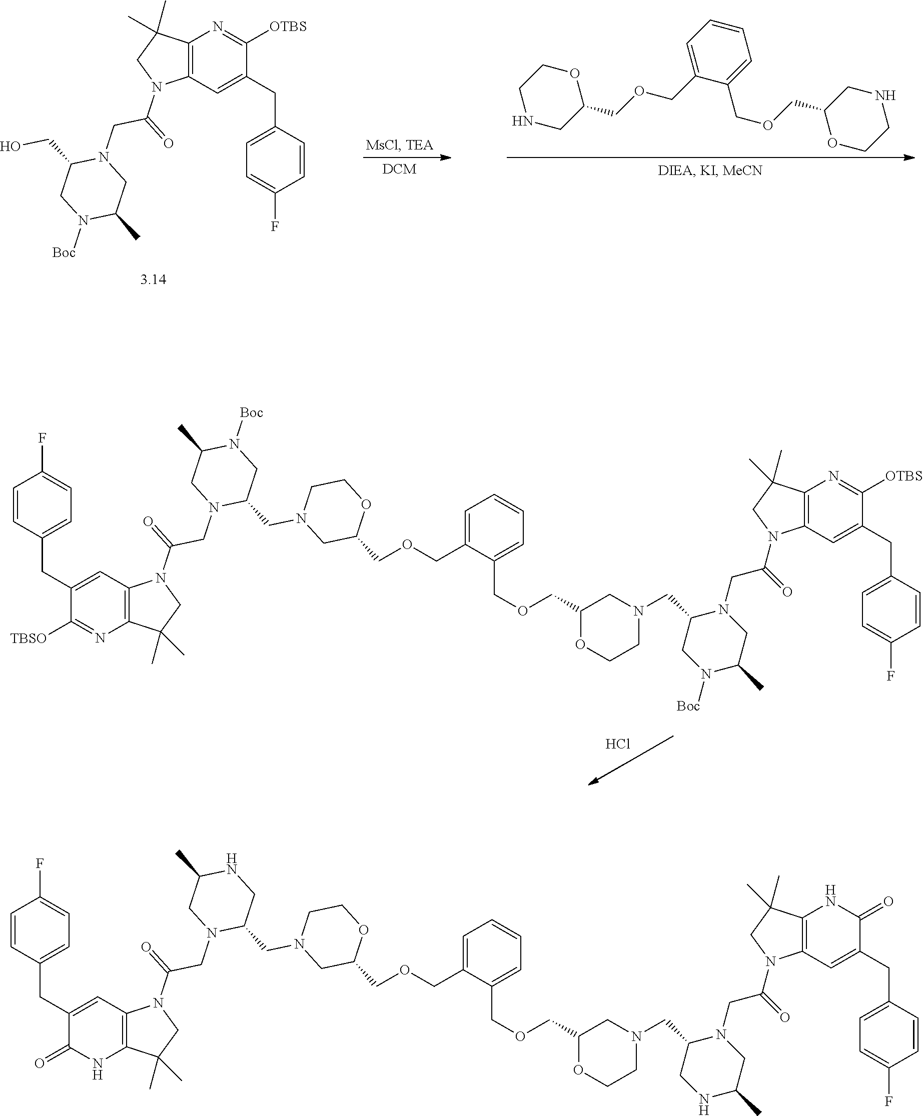

### Step 1 of 2: Synthesis of di-tert-butyl 5,5′-(((2S,2'S)-(((1,2-phenylenebis(methylene))bis(oxy))bis(methylene))bis(morpholine-2,4-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate)

To a solution of tert-butyl (2R,5R)-4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-5-(hydroxymethyl)-2-methylpiperazine-1-carboxylate (0.80 g, 1.22 mmol, 1 eq) and DIEA (393 mg, 3.04 mmol, 530 μL, 2.5 eq) in DCM (10 mL) was added MsCl (167 mg, 1.46 mmol, 113 μL, 1.2 eq) dropwise at 0° C. The mixture was stirred at 15 C for 1 h. TLC (Petroleum ether/Ethyl acetate=0:1, R_{f}=0.79) indicated complete conversion of starting material to a new product. Water (60 mL) was added to the mixture and then extracted with DCM (30 mL*2). The combined organic phases were washed with brine (60 mL), dried over Na₂SO₄, filtered, and concentrated under reduced pressure to give the intermediate product (820 mg, crude) as a yellow oil.

To a solution of the intermediate product (198 mg, 293 umol, 2 eq), 1,2-bis((((S)-morpholin-2-yl)methoxy)methyl)benzene (Linker D-147) (60 mg, 147 umol, 1 eq, 2 HCl), and DIEA (94.7 mg, 733 umol, 128 μL, 5 eq) in MeCN (5 mL) was added KI (48.7 mg, 293 umol, 2 eq). The mixture was stirred at 80° C. for 12 h after which time LCMS indicated formation of a product of target mass. The mixture was filtered and the filtrate was concentrated to dryness. The residue was purified by preparative TLC (Petroleum ether/Ethyl acetate/NH₃·H₂O=50:75:1, R_{f}=0.54) to give the title compound (115 mg, crude) as a yellow solid.

LCMS (ES, m/z): 1614.1 [M+H]⁺

### Step 2 of 2: Synthesis of 1,1′-(2,2′-((3R,3′R,6R,6′R)-(((2S,2'S)-(((1,2-phenylenebis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

To a mixture of di-tert-butyl 5,5′-(((2S,2'S)-(((1,2-phenylenebis(methylene))bis(oxy))bis(methylene))bis(morpholine-2,4-diyl))bis(methylene))(2R,2′R,5S,5'S)-bis(4-(2-(5-((tert-butyldimethylsilyl)oxy)-6-(4-fluorobenzyl)-3,3-dimethyl-2,3-dihydro-1H-pyrrolo[3,2-b]pyridin-1-yl)-2-oxoethyl)-2-methylpiperazine-1-carboxylate) (110 mg, 68.15 umol, 1 eq) in EtOAc (1.5 mL) was added HCl/EtOAc (4 M, 1.5 mL) at 15° C. The mixture was stirred at 15° C. for 2 h after which time LCMS indicated complete consumption of starting material and formation of a product of target mass. The mixture was filtered and the filter cake was dried to give a crude product. The crude product was purified by preparative HPLC (column: Phenomenex Luna C18, 75*30 mm*3 um; mobile phase: [water (0.04% HCl)-ACN]; B %: 5%-35%, 9 min) to give the title compound (25.3 mg, 20.1 umol, 29.5% yield, 100% purity, 2 HCl) as a white solid.

LCMS (ES, m/z): 593.5 [M/2+H]⁺

¹H NMR (400 MHz, METHANOL-d4) δ=7.98 (s, 2H), 7.33 (br dd, J=3.7, 5.2 Hz, 2H), 7.23 (dd, J=3.4, 5.5 Hz, 2H), 7.17 (dd, J=5.6, 8.3 Hz, 4H), 7.01 (t, J=8.7 Hz, 4H), 4.70-4.56 (m, 4H), 4.18 (br d, J=12.1 Hz, 2H), 4.09-3.86 (m, 7H), 3.86-3.68 (m, 12H), 3.66-3.50 (m, 8H), 3.49-3.38 (m, 4H), 3.29-3.16 (m, 7H), 3.15-2.97 (m, 6H), 1.44 (s, 12H), 1.32 (d, J=6.5 Hz, 6H).

Examples 50-113 were prepared according to the same procedure as Example 49, replacing 1,2-bis((((S)-morpholin-2-yl)methoxy)methyl)benzene with the appropriate bis-amine linker. The compounds of Examples 50-113 were found to have characterizing data as set forth below.

### Example 50, 1,1′-(2,2′-((3R,3′R,6R,6′R)-(((2S,2'S)—(([1,1′-biphenyl]-4,4′-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

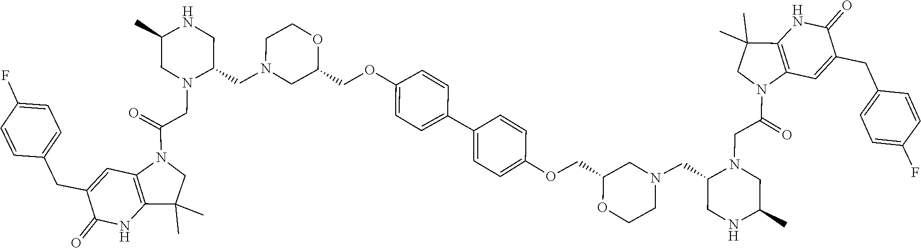 [SMILES: C[C@@H]1CN(CC(=O)N2CC(C)(C)c3[nH]c(=O)c(Cc4ccc(F)cc4)cc32)[C@@H](CN2CCO[C@H](COc3ccc(-c4ccc(OC[C@@H]5CN(C[C@H]6CN[C@H](C)CN6CC(=O)N6CC(C)(C)c7[nH]c(=O)c(Cc8ccc(F)cc8)cc76)CCO5)cc4)cc3)C2)CN1]

LCMS (ES, m/z): 617.6 [M/2+H]⁺

¹H NMR (400 MHz, METHANOL-d₄) δ=8.54 (br s, 1H), 8.22 (s, 2H), 7.37 (br d, J=8.6 Hz, 4H), 7.23 (dd, J=5.6, 8.2 Hz, 4H), 6.97 (t, J=8.7 Hz, 4H), 6.80 (br d, J=8.8 Hz, 4H), 3.97-3.70 (m, 16H), 3.63-3.43 (m, 6H), 3.19-3.04 (m, 6H), 2.97-2.83 (m, 4H), 2.82-2.61 (m, 8H), 2.13 (br d, J=13.6 Hz, 2H), 2.06-1.92 (m, 4H), 1.36 (s, 12H), 1.18 (br d, J=6.5 Hz, 6H).

### Example 51, 1,1′-(2,2′-((3R,3′R,6R,6′R)-(((2S,2'S)—(([1,1′-biphenyl]-3,3′-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

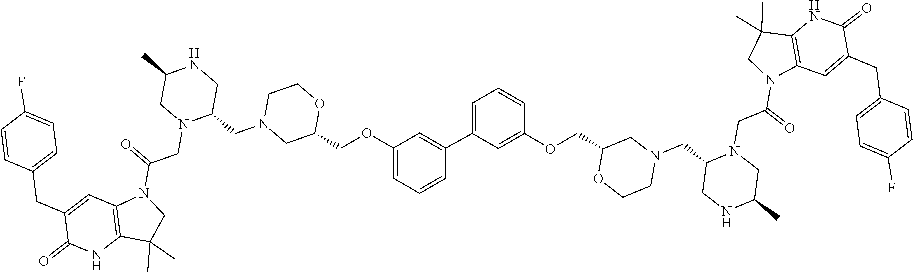 [SMILES: C[C@@H]1CN(CC(=O)N2CC(C)(C)c3[nH]c(=O)c(Cc4ccc(F)cc4)cc32)[C@@H](CN2CCO[C@H](COc3cccc(-c4cccc(OC[C@@H]5CN(C[C@H]6CN[C@H](C)CN6CC(=O)N6CC(C)(C)c7[nH]c(=O)c(Cc8ccc(F)cc8)cc76)CCO5)c4)c3)C2)CN1]

LCMS (ES, m/z): 617.5 [M/2+H]P

¹H NMR (400 MHz, METHANOL-d₄) δ=8.54 (s, 2H), 8.17 (s, 2H), 7.34-7.11 (m, 8H), 7.06 (s, 2H), 6.96 (t, J=8.8 Hz, 4H), 6.82 (dd, J=2.1, 8.2 Hz, 2H), 3.96-3.68 (m, 18H), 3.64-3.57 (m, 2H), 3.49-3.34 (m, 4H), 3.28-3.17 (m, 4H), 3.09-2.93 (m, 4H), 2.84 (br t, J=12.5 Hz, 4H), 2.76-2.62 (m, 4H), 2.16 (br d, J=11.5 Hz, 2H), 2.05-1.95 (m, 4H), 1.37 (d, J=15.3 Hz, 12H), 1.25 (d, J=6.4 Hz, 6H).

### Example 52, 1,1′-(2,2′-((3R,3′R,6R,6′R)-(((2S,2'S)—(([1,1′-biphenyl]-3,4′-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

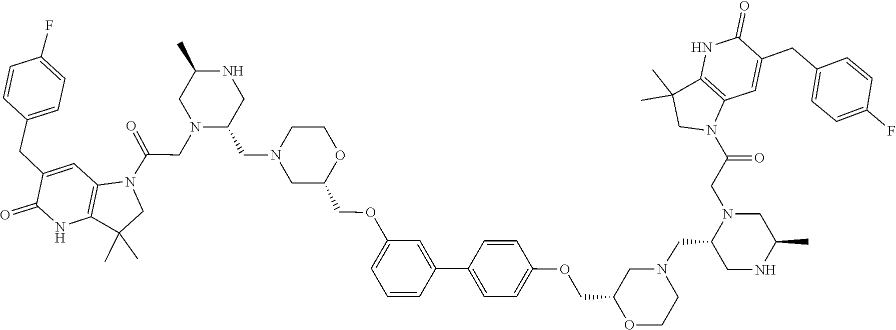 [SMILES: C[C@@H]1CN(CC(=O)N2CC(C)(C)c3[nH]c(=O)c(Cc4ccc(F)cc4)cc32)[C@@H](CN2CCO[C@H](COc3ccc(-c4cccc(OC[C@@H]5CN(C[C@H]6CN[C@H](C)CN6CC(=O)N6CC(C)(C)c7[nH]c(=O)c(Cc8ccc(F)cc8)cc76)CCO5)c4)cc3)C2)CN1]

LCMS (ES, m/z): 617.5 [M/2+H]⁺

¹H NMR (400 MHz, METHANOL-d₄) δ=8.49 (br s, 2H), 8.18 (d, J=10.0 Hz, 2H), 7.47 (d, J=8.8 Hz, 2H), 7.32-7.08 (m, 6H), 7.05-6.88 (m, 5H), 6.86-6.69 (m, 3H), 3.97-3.89 (m, 2H), 3.87-3.71 (m, 15H), 3.67-3.56 (m, 3H), 3.49-3.35 (m, 4H), 3.25 (br d, J=12.5 Hz, 4H), 3.07-2.94 (m, 4H), 2.89-2.63 (m, 8H), 2.17 (br d, J=13.8 Hz, 2H), 2.08-1.94 (m, 4H), 1.44-1.29 (m, 12H), 1.26 (br d, J=6.2 Hz, 6H).

### Example 53, 1,1′-(2,2′-((3R,3′R,6R,6′R)-(((2S,2'S)-(((methylenebis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

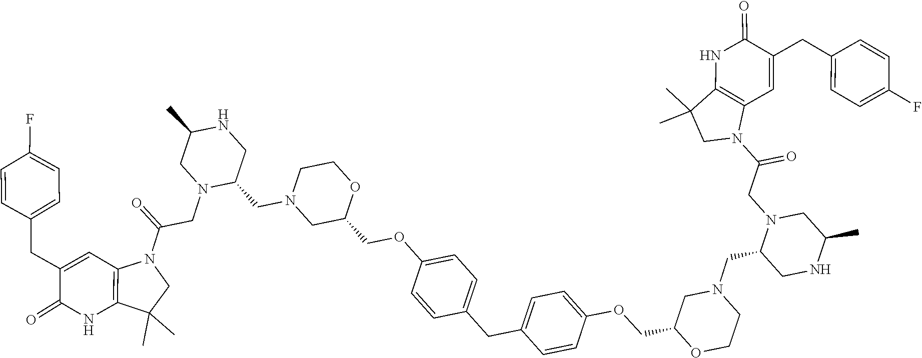 [SMILES: C[C@@H]1CN(CC(=O)N2CC(C)(C)c3[nH]c(=O)c(Cc4ccc(F)cc4)cc32)[C@@H](CN2CCO[C@H](COc3ccc(Cc4ccc(OC[C@@H]5CN(C[C@H]6CN[C@H](C)CN6CC(=O)N6CC(C)(C)c7[nH]c(=O)c(Cc8ccc(F)cc8)cc76)CCO5)cc4)cc3)C2)CN1]

LCMS (ES, m/z): 624.5 [M/2+H]⁺

¹H NMR (400 MHz, METHANOL-d₄) δ ppm 8.53 (s, 2H) 8.15 (s, 2H) 7.19 (br dd, J=8.03, 5.52 Hz, 4H) 6.89-7.06 (m, 8H) 6.72 (br d, J=8.53 Hz, 4H) 3.66-3.95 (m, 21H) 3.54-3.62 (m, 2H) 3.35-3.44 (m, 4H) 3.22 (br d, J=10.04 Hz, 4H) 2.97 (br d, J=5.02 Hz, 4H) 2.59-2.87 (m, 8H) 2.16 (br d, J=11.04 Hz, 2H) 1.88-2.06 (m, 4H) 1.36 (br d, J=3.01 Hz, 11H) 1.24 (br d, J=6.53 Hz, 6H).

### Example 54, 1,1′-(2,2′-((3R,3′R,6R,6′R)-(((2S,2'S)-(((carbonylbis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

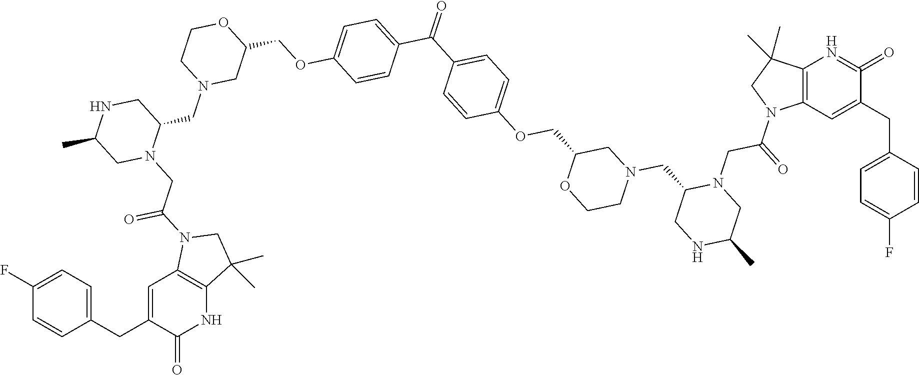

LCMS (ES, m/z): 631.5 [M/2+H]⁺

¹H NMR (400 MHz, METHANOL-d₄) δ=8.19 (s, 2H), 7.71 (d, J=8.8 Hz, 4H), 7.21 (dd, J=5.4, 8.6 Hz, 4H), 7.02-6.89 (m, 8H), 3.99 (br dd, J=5.8, 10.3 Hz, 2H), 3.92-3.84 (m, 4H), 3.83-3.58 (m, 14H), 3.44 (br t, J=10.6 Hz, 4H), 3.16 (br d, J=11.5 Hz, 4H), 2.99-2.77 (m, 8H), 2.76-2.62 (m, 4H), 2.16 (br d, J=11.4 Hz, 2H), 2.01 (br t, J=10.6 Hz, 4H), 1.38 (d, J=7.0 Hz, 12H), 1.21 (br d, J=6.4 Hz, 6H).

### Example 55, 1,1′-(2,2′-((3R,3′R,6R,6′R)-(((2S,2'S)-(((oxybis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

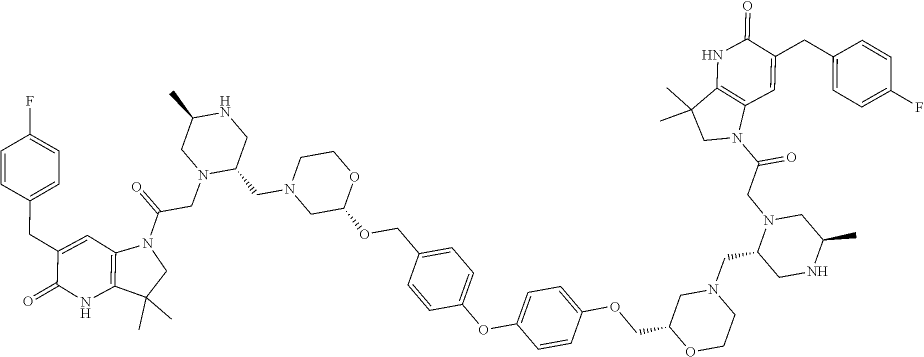 [SMILES: C[C@@H]1CN(CC(=O)N2CC(C)(C)c3[nH]c(=O)c(Cc4ccc(F)cc4)cc32)[C@@H](CN2CCO[C@H](COc3ccc(Oc4ccc(CO[C@@H]5CN(C[C@H]6CN[C@H](C)CN6CC(=O)N6CC(C)(C)c7[nH]c(=O)c(Cc8ccc(F)cc8)cc76)CCO5)cc4)cc3)C2)CN1]

LCMS (ES, m/z): 625.6 [M/2+H]⁺

¹H NMR (400 MHz, METHANOL-d₄) δ=8.55 (s, 1H), 8.18 (s, 1H), 7.21 (br dd, J=5.5, 8.3 Hz, 4H), 7.02-6.91 (m, 4H), 6.88-6.72 (m, 8H), 3.91-3.70 (m, 16H), 3.67 (s, 4H), 3.46-3.34 (m, 2H), 3.27 (br d, J=10.3 Hz, 2H), 3.22-3.07 (m, 4H), 2.98-2.74 (m, 8H), 2.74-2.59 (m, 4H), 2.14 (br d, J=11.3 Hz, 2H), 2.05-1.91 (m, 4H), 1.38 (br d, J=2.5 Hz, 12H), 1.20 (br d, J=6.4 Hz, 6H).

### Example 56, 1,1′-(2,2′-((3R,3′R,6R,6′R)-(((2S,2'S)-(((propane-2,2-diylbis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

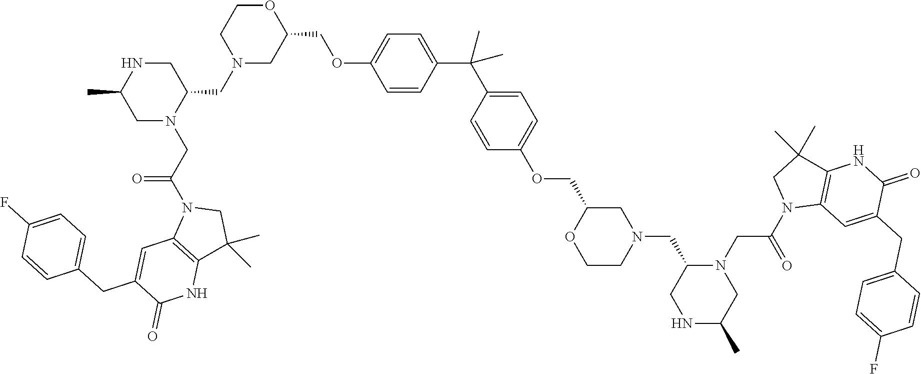 [SMILES: C[C@@H]1CN(CC(=O)N2CC(C)(C)c3[nH]c(=O)c(Cc4ccc(F)cc4)cc32)[C@@H](CN2CCO[C@H](COc3ccc(C(C)(C)c4ccc(OC[C@@H]5CN(C[C@H]6CN[C@H](C)CN6CC(=O)N6CC(C)(C)c7[nH]c(=O)c(Cc8ccc(F)cc8)cc76)CCO5)cc4)cc3)C2)CN1]

LCMS (ES, m/z): 638.6 [M/2+H]⁺

¹H NMR (400 MHz, METHANOL-d₄) δ=8.16 (s, 2H), 7.18 (dd, J=5.5, 8.5 Hz, 4H), 7.08 (d, J=8.8 Hz, 4H), 6.94 (t, J=8.8 Hz, 4H), 6.71 (d, J=8.9 Hz, 4H), 3.92-3.55 (m, 21H), 3.42 (br t, J=10.9 Hz, 2H), 3.29-3.24 (m, 3H), 3.12 (br d, J=10.9 Hz, 4H), 2.97-2.74 (m, 8H), 2.72-2.57 (m, 4H), 2.13 (br d, J=11.0 Hz, 2H), 2.05-1.91 (m, 4H), 1.59 (s, 6H), 1.38 (d, J=6.0 Hz, 12H), 1.18 (d, J=6.4 Hz, 6H).

### Example 57, 1,1′-(2,2′-((3R,3′R,6R,6′R)-(((2S,2'S)-(((sulfonylbis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

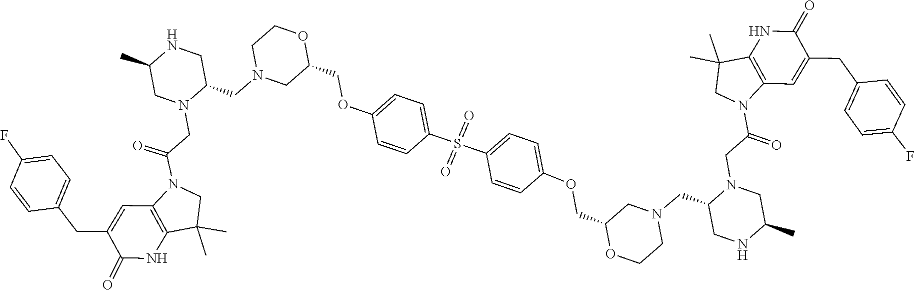 [SMILES: C[C@@H]1CN(CC(=O)N2CC(C)(C)c3[nH]c(=O)c(Cc4ccc(F)cc4)cc32)[C@@H](CN2CCO[C@H](COc3ccc(S(=O)(=O)c4ccc(OC[C@@H]5CN(C[C@H]6CN[C@H](C)CN6CC(=O)N6CC(C)(C)c7[nH]c(=O)c(Cc8ccc(F)cc8)cc76)CCO5)cc4)cc3)C2)CN1]

LCMS (ES, m/z): 649.5 [M/2+H]⁺

¹H NMR (400 MHz, METHANOL-d₄) δ=8.53 (br s, 2H), 8.14 (s, 2H), 7.81 (d, J=8.9 Hz, 4H), 7.16 (dd, J=5.5, 8.5 Hz, 4H), 7.00-6.87 (m, 8H), 3.96-3.90 (m, 2H), 3.88-3.80 (m, 4H), 3.79-3.64 (m, 12H), 3.63-3.54 (m, 2H), 3.42-3.34 (m, 4H), 3.24-3.15 (m, 4H), 3.01-2.89 (m, 4H), 2.85-2.60 (m, 8H), 2.16 (br d, J=10.5 Hz, 2H), 2.07-1.91 (m, 4H), 1.36 (d, J=10.6 Hz, 12H), 1.22 (d, J=6.5 Hz, 6H).

### Example 58, 1,1′-(2,2′-((3R,3′R,6R,6′R)-(((2S,2'S)-(((ethane-1,2-diylbis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

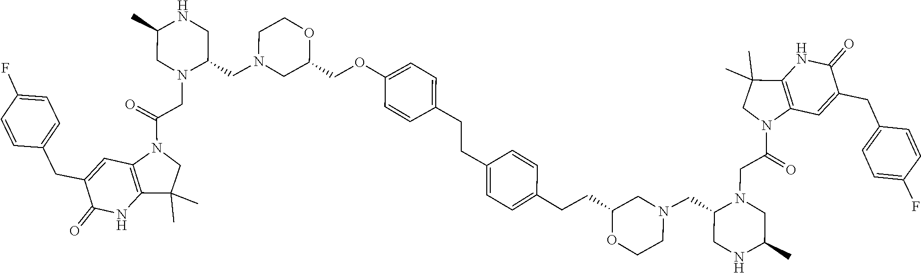 [SMILES: C[C@@H]1CN(CC(=O)N2CC(C)(C)c3[nH]c(=O)c(Cc4ccc(F)cc4)cc32)[C@@H](CN2CCO[C@H](CCc3ccc(CCc4ccc(OC[C@@H]5CN(C[C@H]6CN[C@H](C)CN6CC(=O)N6CC(C)(C)c7[nH]c(=O)c(Cc8ccc(F)cc8)cc76)CCO5)cc4)cc3)C2)CN1]

LCMS (ES, m/z): 631.6 [M/2+H]⁺

¹H NMR (400 MHz, METHANOL-d4) δ=8.53 (s, 2H), 8.15 (s, 2H), 7.20 (dd, J=5.6, 8.3 Hz, 4H), 7.02-6.91 (m, 8H), 6.73-6.63 (m, 4H), 3.86-3.71 (m, 16H), 3.62-3.53 (m, 2H), 3.46-3.34 (m, 4H), 3.23 (br d, J=9.9 Hz, 4H), 3.06-2.93 (m, 4H), 2.88-2.60 (m, 12H), 2.21-2.12 (m, 2H), 2.05-1.93 (m, 4H), 1.37 (d, J=5.3 Hz, 12H), 1.24 (br d, J=6.1 Hz, 6H).

### Example 59, 1,2-bis(4-(((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-1)methyl)morpholin-2-yl)methoxy)phenyl)ethane-1,2-dione

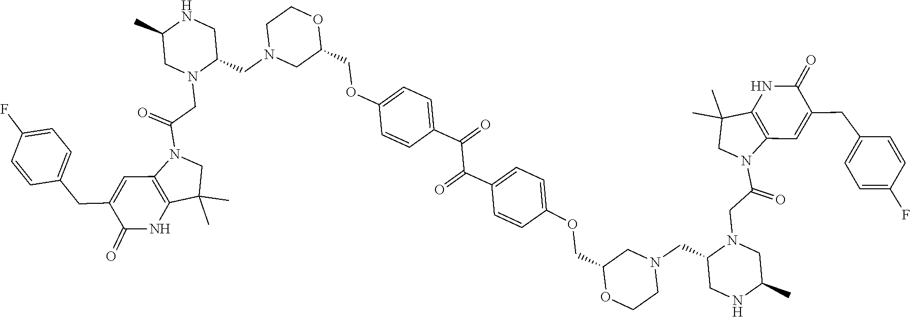 [SMILES: C[C@@H]1CN(CC(=O)N2CC(C)(C)c3[nH]c(=O)c(Cc4ccc(F)cc4)cc32)[C@@H](CN2CCO[C@H](COc3ccc(C(=O)C(=O)c4ccc(OC[C@@H]5CN(C[C@H]6CN[C@H](C)CN6CC(=O)N6CC(C)(C)c7[nH]c(=O)c(Cc8ccc(F)cc8)cc76)CCO5)cc4)cc3)C2)CN1]

LCMS (ES, m/z): 645.6 [M/2+H]⁺

¹H NMR (400 MHz, METHANOL-d₄) δ=8.52 (br s, 2H), 8.20-8.14 (m, 2H), 7.94-7.76 (m, 4H), 7.24-7.13 (m, 4H), 7.03-6.87 (m, 8H), 4.05-3.95 (m, 2H), 3.91-3.70 (m, 15H), 3.66-3.56 (m, 2H), 3.45-3.34 (m, 4H), 3.25-3.14 (m, 4H), 3.04-2.96 (m, 3H), 2.86-2.63 (m, 8H), 2.22-2.14 (m, 2H), 2.05-1.92 (m, 4H), 1.42-1.31 (m, 12H), 1.25 (br d, J=6.4 Hz, 6H).

### Example 60, 1,1′-(2,2′-((3R,3′R,6R,6′R)-(((2S,2'S)-((((1,3-phenylenebis(oxy))bis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

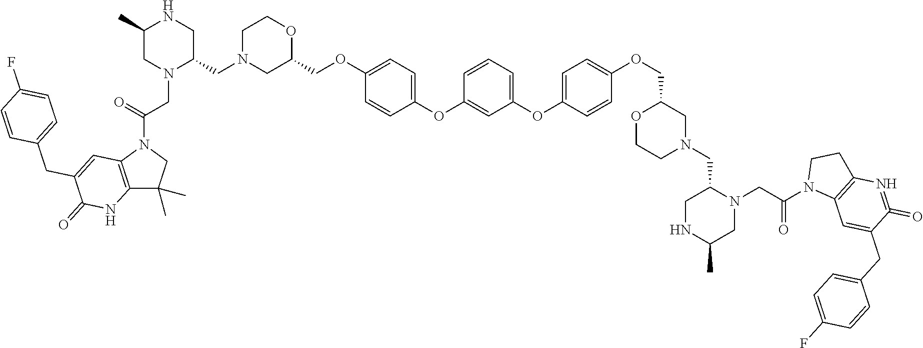

LCMS (ES, m/z): 671.5 [M/2+H]⁺

¹H NMR (400 MHz, METHANOL-d₄) δ=8.53 (s, 2H), 8.16 (s, 2H), 7.25-7.15 (m, 5H), 7.00-6.81 (m, 12H), 6.55 (dd, J=2.4, 8.3 Hz, 2H), 6.45 (t, J=2.3 Hz, 1H), 3.88-3.71 (m, 18H), 3.63-3.54 (m, 2H), 3.46-3.35 (m, 4H), 3.28-3.20 (m, 4H), 3.07-2.94 (m, 4H), 2.85-2.63 (m, 8H), 2.23-2.13 (m, 2H), 2.05-1.95 (m, 4H), 1.38 (d, J=2.8 Hz, 12H), 1.25 (d, J=6.5 Hz, 6H).

### Example 61, 1,1′-(2,2′-((3R,3′R,6R,6′R)-(((2S,2'S)-((((1,3-phenylenebis(propane-2,2-diyl))bis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

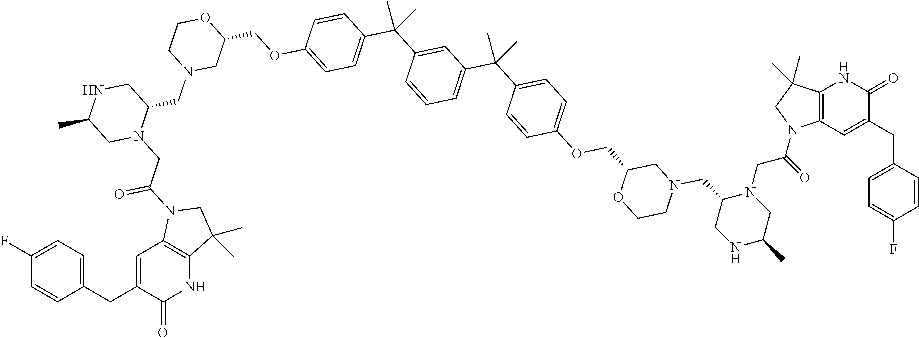 [SMILES: C[C@@H]1CN(CC(=O)N2CC(C)(C)c3[nH]c(=O)c(Cc4ccc(F)cc4)cc32)[C@@H](CN2CCO[C@H](COc3ccc(C(C)(C)c4cccc(C(C)(C)c5ccc(OC[C@@H]6CN(C[C@H]7CN[C@H](C)CN7CC(=O)N7CC(C)(C)c8[nH]c(=O)c(Cc9ccc(F)cc9)cc87)CCO6)cc5)c4)cc3)C2)CN1]

LCMS (ES, m/z): 697.7 [M/2+H]⁺

¹H NMR (400 MHz, METHANOL-d₄) δ=8.08-8.01 (m, 2H), 7.23-7.05 (m, 10H), 7.03-6.94 (m, 6H), 6.75 (br d, J=8.6 Hz, 4H), 4.19-3.58 (m, 26H), 3.55-3.39 (m, 4H), 3.29-2.95 (m, 16H), 1.56 (s, 12H), 1.47-1.38 (m, 12H), 1.30 (d, J=6.5 Hz, 6H).

### Example 62, 1,1′-(2,2′-((3R,3′R,6R,6′R)-(((2S,2'S)-((((1,4-phenylenebis(propane-2,2-diyl))bis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

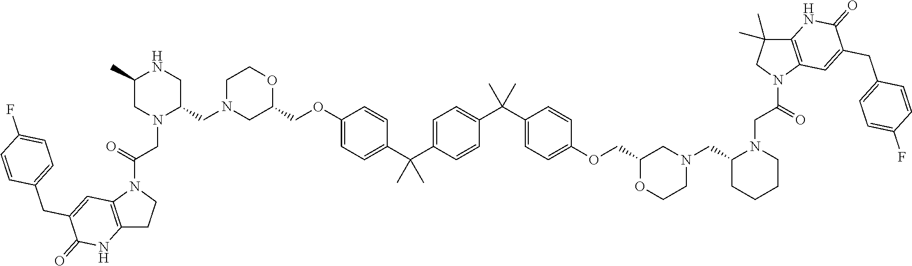 [SMILES: C[C@@H]1CN(CC(=O)N2CCc3[nH]c(=O)c(Cc4ccc(F)cc4)cc32)[C@@H](CN2CCO[C@H](COc3ccc(C(C)(C)c4ccc(C(C)(C)c5ccc(OC[C@@H]6CN(C[C@H]7CCCCN7CC(=O)N7CC(C)(C)c8[nH]c(=O)c(Cc9ccc(F)cc9)cc87)CCO6)cc5)cc4)cc3)C2)CN1]

LCMS (ES, m/z): 697.6 [M/2+H]⁺

¹H NMR (400 MHz, METHANOL-d4) δ=8.53 (s, 2H), 8.17-8.12 (m, 2H), 7.17 (dd, J=5.6, 8.1 Hz, 4H), 7.13-7.02 (m, 8H), 6.93 (br t, J=8.7 Hz, 4H), 6.72 (br d, J=8.6 Hz, 4H), 3.90-3.81 (m, 4H), 3.81-3.69 (m, 13H), 3.63-3.54 (m, 2H), 3.45-3.34 (m, 4H), 3.21 (br d, J=10.6 Hz, 4H), 3.05-2.92 (m, 4H), 2.85-2.77 (m, 4H), 2.76-2.61 (m, 4H), 2.22-2.11 (m, 2H), 2.06-1.94 (m, 4H), 1.58 (s, 11H), 1.37 (br d, J=7.4 Hz, 12H), 1.28-1.19 (m, 6H).

### Example 63, 1,1′-(2,2′-((3R,3′R,6R,6′R)-(((2S,2'S)-((hexa-2,4-diyne-1,6-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

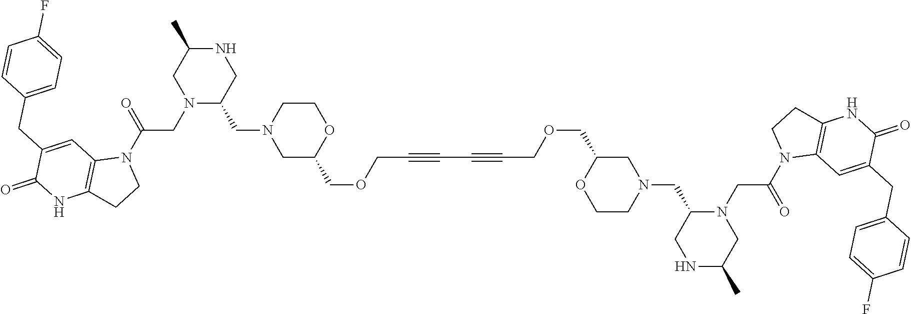 [SMILES: C[C@@H]1CN(CC(=O)N2CCc3[nH]c(=O)c(Cc4ccc(F)cc4)cc32)[C@@H](CN2CCO[C@H](COCC#CC#CCOC[C@@H]3CN(C[C@H]4CN[C@H](C)CN4CC(=O)N4CCc5[nH]c(=O)c(Cc6ccc(F)cc6)cc54)CCO3)C2)CN1]

LCMS (ES, m/z): 579.5 [M/2+H]⁺

¹H NMR (400 MHz, METHANOL-d₄) δ=8.03-7.99 (m, 2H), 7.27-7.21 (m, 4H), 7.09-7.03 (m, 4H), 4.29-4.24 (m, 4H), 4.13-4.05 (m, 2H), 4.00-3.94 (m, 2H), 3.89-3.39 (m, 25H), 3.27-3.17 (m, 8H), 3.12-2.98 (m, 6H), 2.94-2.81 (m, 3H), 1.42 (d, J=7.9 Hz, 12H), 1.30 (d, J=6.1 Hz, 6H).

### Example 64, 1,1′-(2,2′-((3R,3′R,6R,6′R)-(((2S,2'S)-((hexane-1,6-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

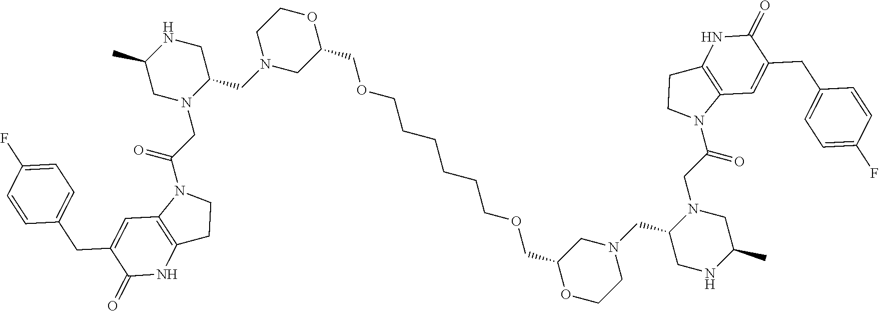 [SMILES: C[C@@H]1CN(CC(=O)N2CCc3[nH]c(=O)c(Cc4ccc(F)cc4)cc32)[C@@H](CN2CCO[C@H](COCCCCCCOC[C@@H]3CN(C[C@H]4CN[C@H](C)CN4CC(=O)N4CCc5[nH]c(=O)c(Cc6ccc(F)cc6)cc54)CCO3)C2)CN1]

LCMS (ES, m/z): 583.4 [M/2+H]⁺

¹H NMR (400 MHz, METHANOL-d4) δ=8.07-8.02 (m, 2H), 7.27-7.21 (m, 4H), 7.06 (t, J=8.8 Hz, 4H), 4.12-4.03 (m, 2H), 3.99-3.94 (m, 2H), 3.91-3.84 (m, 3H), 3.82-3.78 (m, 6H), 3.75-3.62 (m, 6H), 3.53-3.36 (m, 13H), 3.27-3.15 (m, 8H), 3.11-3.00 (m, 6H), 2.92-2.90 (m, 1H), 2.92-2.73 (m, 3H), 1.48-1.40 (m, 16H), 1.32-1.28 (m, 6H), 1.26 (br d, J=6.9 Hz, 4H).

### Example 65, 1,1′-(2,2′-((3R,3′R,6R,6′R)-(((2S,2'S)-(((1,4-phenylenebis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

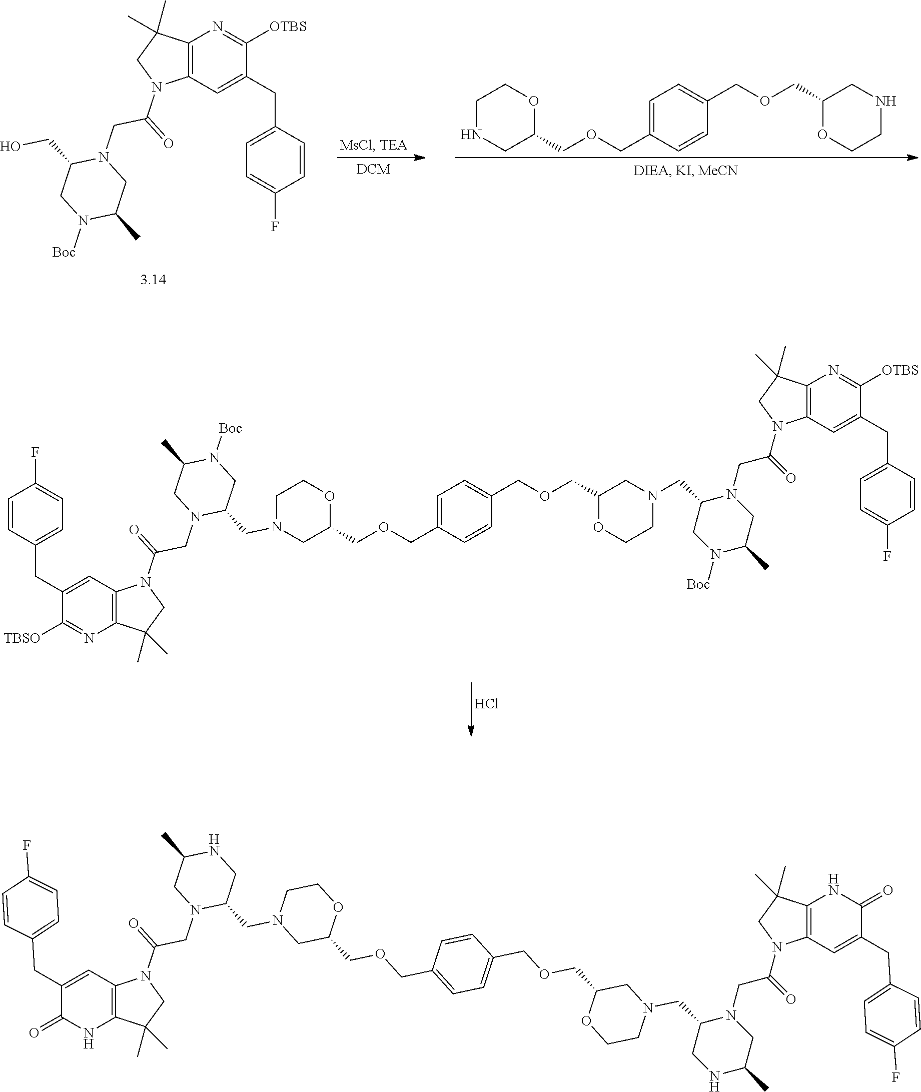

LCMS (ES, m/z): 593.5 [M/2+H]⁺

¹H NMR (400 MHz, METHANOL-d4) δ=8.03-7.95 (m, 2H), 7.25-7.12 (m, 8H), 7.03-6.94 (m, 4H), 4.51-4.42 (m, 4H), 4.14-4.03 (m, 2H), 3.99-3.92 (m, 2H), 3.90-3.82 (m, 2H), 3.82-3.63 (m, 14H), 3.63-3.55 (m, 2H), 3.55-3.37 (m, 7H), 3.27-3.15 (m, 8H), 3.14-2.97 (m, 7H), 2.96-2.77 (m, 4H), 1.46-1.36 (m, 12H), 1.30 (d, J=6.5 Hz, 6H).

### Example 66, 1,1′-(2,2′-((3R,3′R,6R,6′R)-(((2S,2'S)-(((methylenebis(2,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

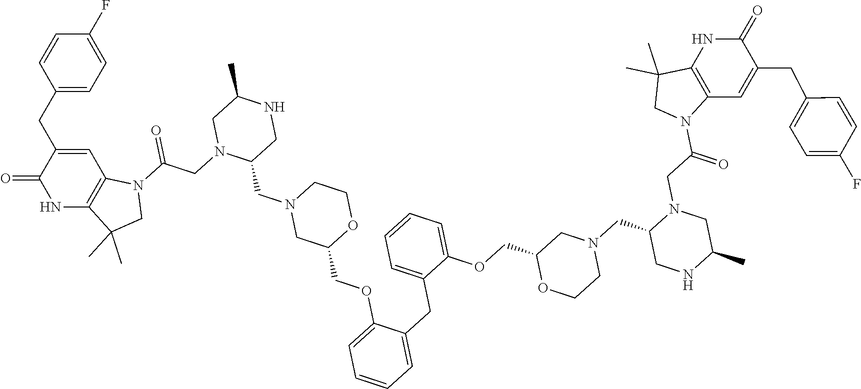 [SMILES: C[C@@H]1CN(CC(=O)N2CC(C)(C)c3[nH]c(=O)c(Cc4ccc(F)cc4)cc32)[C@@H](CN2CCO[C@H](COc3ccccc3Cc3ccccc3OC[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 624.6 [M/2+H]⁺

¹H NMR (400 MHz, METHANOL-d₄) δ=8.59-8.50 (m, 1H), 8.36-8.25 (m, 2H), 7.27-6.99 (m, 8H), 6.85-6.59 (m, 8H), 3.96-3.64 (m, 12H), 3.64-3.39 (m, 12H), 3.16-3.00 (m, 4H), 2.98-2.74 (m, 8H), 2.70-2.51 (m, 6H), 2.07-1.96 (m, 2H), 1.96-1.78 (m, 4H), 1.46-1.23 (m, 12H), 1.15 (d, J=6.4 Hz, 5H).

### Example 67, 1,1′-(2,2′-((3R,3′R,6R,6′R)-(((2S,2'S)—(([1,1′-biphenyl]-2,2′-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

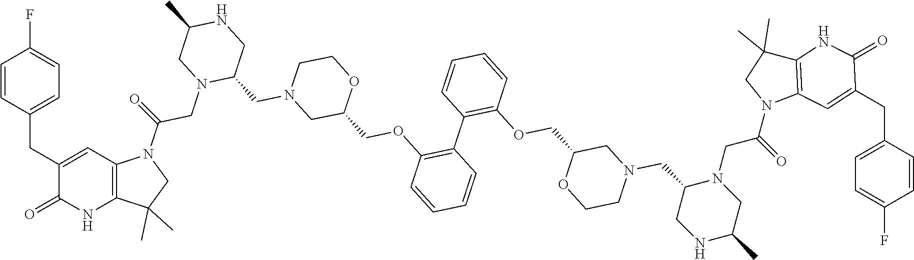 [SMILES: C[C@@H]1CN(CC(=O)N2CC(C)(C)c3[nH]c(=O)c(Cc4ccc(F)cc4)cc32)[C@@H](CN2CCO[C@H](COc3ccccc3-c3ccccc3OC[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 617.6 [M/2+H]⁺

¹H NMR (400 MHz, METHANOL-d₄) δ=8.65-8.44 (m, 2H), 8.16 (s, 2H), 7.29-7.15 (m, 8H), 7.03-6.86 (m, 8H), 3.84-3.77 (m, 5H), 3.74-3.60 (m, 6H), 3.54-3.48 (m, 3H), 3.42-3.34 (m, 2H), 3.27 (br d, J=10.6 Hz, 4H), 3.22-3.12 (m, 4H), 3.10-2.91 (m, 6H), 2.75 (br d, J=11.0 Hz, 2H), 2.70-2.57 (m, 4H), 2.51-2.39 (m, 2H), 2.12-2.02 (m, 2H), 1.90-1.76 (m, 4H), 1.33-1.17 (m, 12H), 1.16-1.02 (m, 6H).

### Example 68, 1,1′-(2,2′-((3R,3′R,6R,6′R)-(((2S,2'S)-(((oxybis(2,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

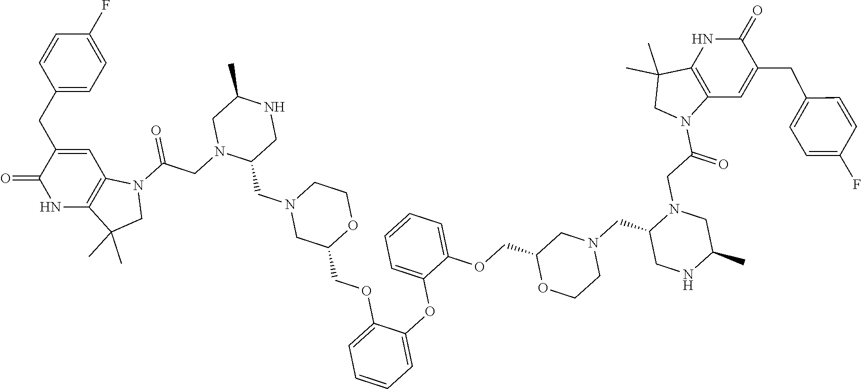 [SMILES: C[C@@H]1CN(CC(=O)N2CC(C)(C)c3[nH]c(=O)c(Cc4ccc(F)cc4)cc32)[C@@H](CN2CCO[C@H](COc3ccccc3Oc3ccccc3OC[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 625.5 [M/2+H]⁺

¹H NMR (400 MHz, METHANOL-d₄) δ=7.99-7.88 (m, 2H), 7.20-6.99 (m, 12H), 6.93 (br t, J=7.6 Hz, 2H), 6.81-6.70 (m, 2H), 4.14 (br d, J=3.9 Hz, 6H), 4.05-3.92 (m, 6H), 3.88-3.62 (m, 13H), 3.61-3.45 (m, 4H), 3.43-3.33 (m, 5H), 3.28-3.15 (m, 6H), 3.08-2.84 (m, 6H), 1.46-1.40 (m, 12H), 1.33 (br d, J=6.4 Hz, 6H).

### Example 69, 1,1′-(2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

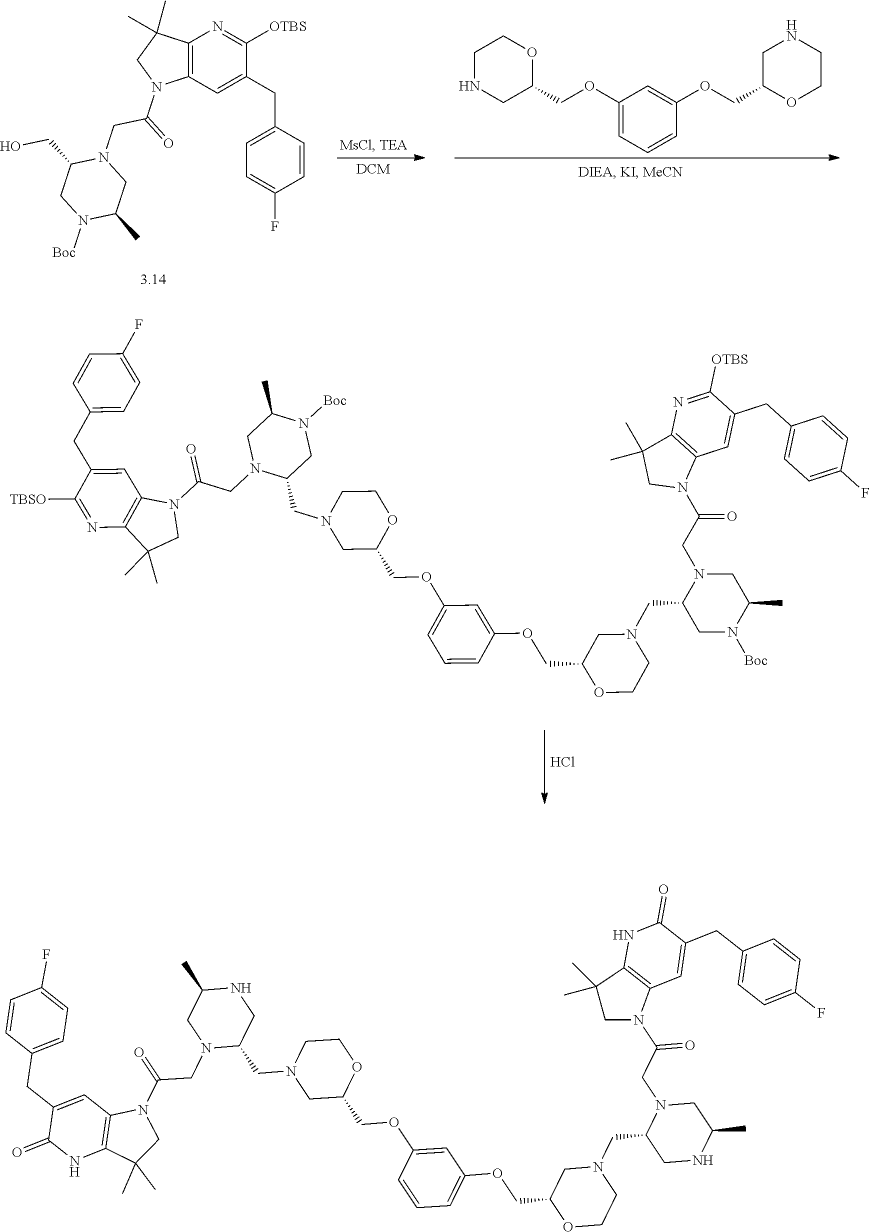

LCMS (ES, m/z): 1157.7 [M+H]⁺

¹H NMR (400 MHz, CD₃OD, 297 K) δ (ppm)=8.08-7.94 (m, 2H), 7.24-7.12 (m, 5H), 7.05-6.92 (m, 4H), 6.55 (dd, J=2.3, 8.3 Hz, 2H), 6.49-6.38 (m, 1H), 4.18-4.09 (m, 2H), 4.08-3.93 (m, 8H), 3.88 (br d, J=10.8 Hz, 3H), 3.82-3.71 (m, 10H), 3.71-3.61 (m, 2H), 3.56-3.43 (m, 4H), 3.42-3.35 (m, 2H), 3.22 (br d, J=18.7 Hz, 6H), 3.15-2.98 (m, 7H), 2.97-2.72 (m, 3H), 1.41 (d, J=13.4 Hz, 11H), 1.30 (br d, J=6.5 Hz, 6H).

### Example 70, 1,1′-(2,2′-((3R,3′R,6R,6′R)-(((2S,2'S)-((1,4-phenylenebis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

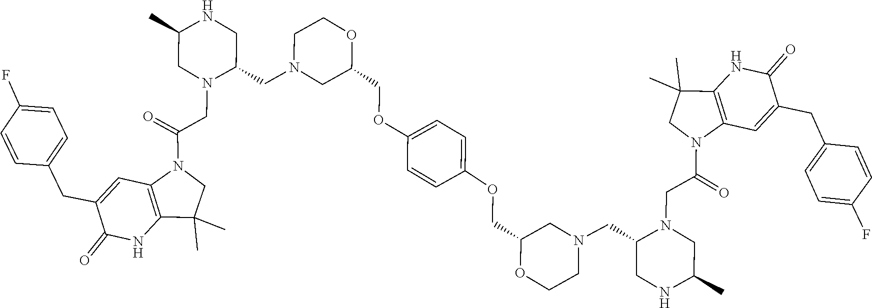 [SMILES: C[C@@H]1CN(CC(=O)N2CC(C)(C)c3[nH]c(=O)c(Cc4ccc(F)cc4)cc32)[C@@H](CN2CCO[C@H](COc3ccc(OC[C@@H]4CN(C[C@H]5CN[C@H](C)CN5CC(=O)N5CC(C)(C)c6[nH]c(=O)c(Cc7ccc(F)cc7)cc65)CCO4)cc3)C2)CN1]

LCMS (ES, m/z): 579.5 [M/2+H]⁺

¹H NMR (400 MHz, METHANOL-d4) δ=8.54 (s, 1H), 8.25 (s, 1H), 7.25-7.17 (m, 4H), 6.96 (t, J=8.8 Hz, 4H), 6.55 (s, 4H), 3.92-3.72 (m, 14H), 3.38-3.38 (m, 1H), 3.69-3.36 (m, 7H), 3.29-3.14 (m, 6H), 3.03-2.65 (m, 12H), 2.13 (br d, J=13.0 Hz, 2H), 2.05-1.92 (m, 4H), 1.49-1.33 (m, 12H), 1.25 (br d, J=6.5 Hz, 6H).

### Example 71, 1,1′-(2,2′-((3R,3′R,6R,6′R)-(((2S,2'S)-((1,2-phenylenebis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

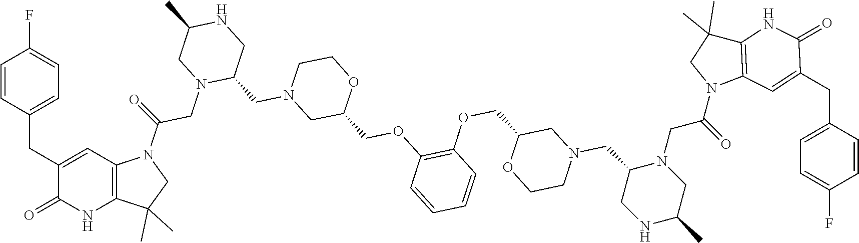 [SMILES: C[C@@H]1CN(CC(=O)N2CC(C)(C)c3[nH]c(=O)c(Cc4ccc(F)cc4)cc32)[C@@H](CN2CCO[C@H](COc3ccccc3OC[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 579.6 [M/2+H]⁺

¹H NMR (400 MHz, METHANOL-d4) δ=8.53 (s, 2H), 8.14 (s, 2H), 7.19 (dd, J=5.5, 8.4 Hz, 4H), 6.96 (t, J=8.8 Hz, 4H), 6.86 (br d, J=2.4 Hz, 4H), 3.91 (dd, J=5.6, 10.4 Hz, 2H), 3.84-3.70 (m, 16H), 3.61-3.53 (m, 2H), 3.45 (br dd, J=9.0, 9.9 Hz, 2H), 3.39-3.33 (m, 2H), 3.27-3.19 (m, 4H), 3.08-2.94 (m, 4H), 2.85-2.60 (m, 8H), 2.15 (s, 2H), 2.06-1.93 (m, 4H), 1.33 (d, J=15.6 Hz, 12H), 1.26 (d, J=6.5 Hz, 6H).

### Example 72, 1,1′-(2,2′-((3R,3′R,6R,6′R)-(((2S,2'S)-(((2-fluoro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

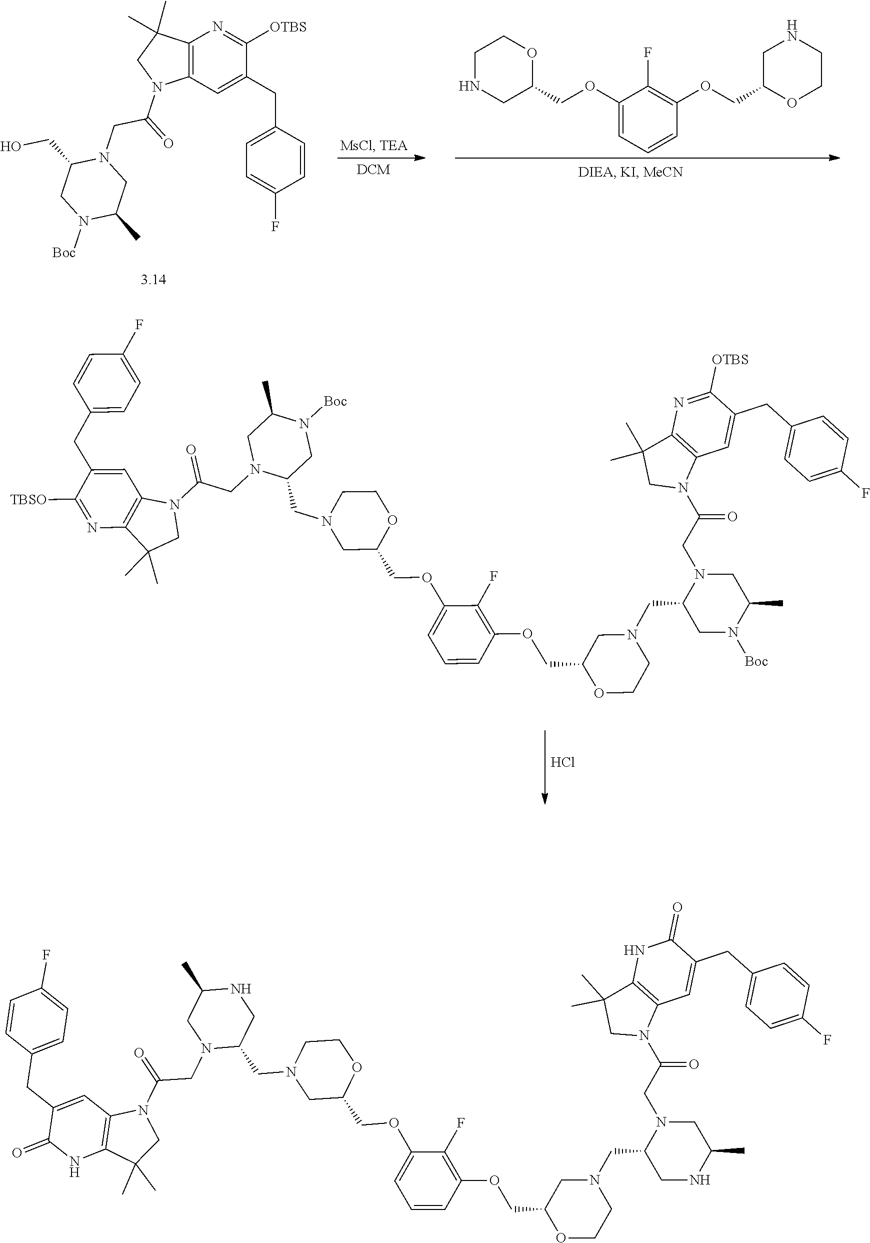

LCMS (ES, m/z): 588.5 [M/2+H]⁺

¹H NMR (400 MHz, METHANOL-d₄) δ=8.54 (s, 2H), 8.21 (s, 2H), 7.22 (dd, J=5.6, 8.3 Hz, 4H), 7.00-6.86 (m, 5H), 6.59 (t, J=7.8 Hz, 2H), 3.95-3.68 (m, 18H), 3.57 (br dd, J=4.3, 5.6 Hz, 2H), 3.44-3.33 (m, 4H), 3.23-3.12 (m, 4H), 3.03-2.91 (m, 4H), 2.84 (br d, J=10.8 Hz, 2H), 2.78-2.61 (m, 6H), 2.13 (br d, J=12.6 Hz, 2H), 1.96 (br t, J=10.9 Hz, 4H), 1.38 (d, J=9.5 Hz, 12H), 1.22 (d, J=6.4 Hz, 6H).

### Example 73, 1,1′-(2,2′-((3R,3′R,6R,6′R)-(((2S,2'S)-(((5-fluoro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

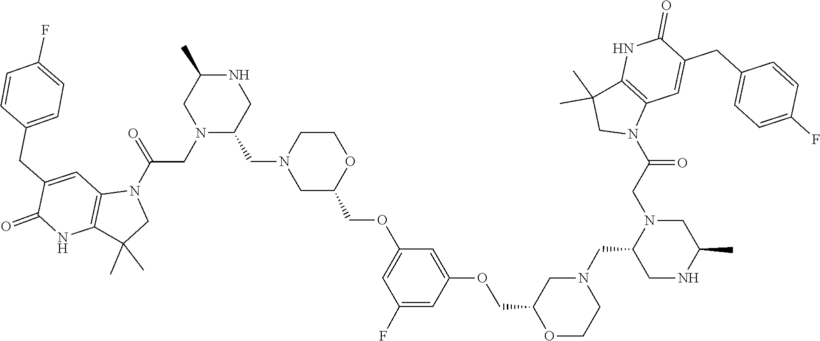 [SMILES: C[C@@H]1CN(CC(=O)N2CC(C)(C)c3[nH]c(=O)c(Cc4ccc(F)cc4)cc32)[C@@H](CN2CCO[C@H](COc3cc(F)cc(OC[C@@H]4CN(C[C@H]5CN[C@H](C)CN5CC(=O)N5CC(C)(C)c6[nH]c(=O)c(Cc7ccc(F)cc7)cc65)CCO4)c3)C2)CN1]

LCMS (ES, m/z): 588.6 [M/2+H]⁺

¹H NMR (400 MHz, METHANOL-d₄) δ=8.54 (br s, 2H), 8.21-8.16 (m, 2H), 7.25-7.18 (m, 4H), 7.00-6.92 (m, 4H), 6.28-6.21 (m, 2H), 6.17-6.12 (m, 1H), 3.91-3.66 (m, 18H), 3.58-3.49 (m, 2H), 3.44-3.34 (m, 3H), 3.24-3.13 (m, 4H), 2.99-2.91 (m, 4H), 2.84 (br d, J=11.3 Hz, 2H), 2.78-2.60 (m, 6H), 2.18-2.10 (m, 2H), 2.01-1.93 (m, 3H), 1.42-1.34 (m, 12H), 1.26-1.18 (m, 6H).

### Example 74, 1,1′-(2,2′-((3R,3′R,6R,6′R)-(((2S,2'S)-(((2,4-difluoro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

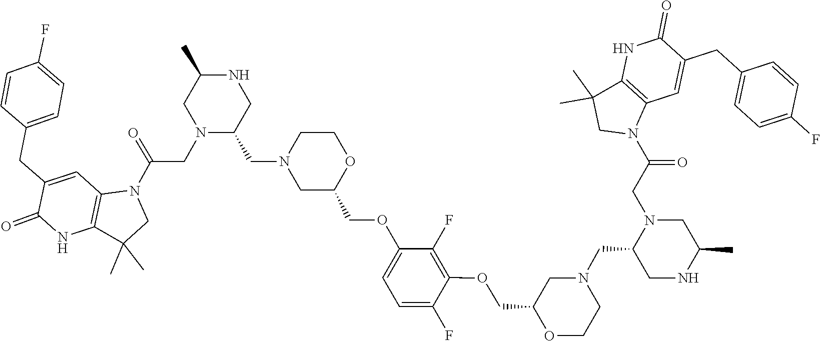 [SMILES: C[C@@H]1CN(CC(=O)N2CC(C)(C)c3[nH]c(=O)c(Cc4ccc(F)cc4)cc32)[C@@H](CN2CCO[C@H](COc3ccc(F)c(OC[C@@H]4CN(C[C@H]5CN[C@H](C)CN5CC(=O)N5CC(C)(C)c6[nH]c(=O)c(Cc7ccc(F)cc7)cc65)CCO4)c3F)C2)CN1]

LCMS (ES, m/z): 597.6 [M/2+H]⁺

¹H NMR (400 MHz, METHANOL-d₄) δ=8.55 (s, 1H), 8.17 (d, J=13.6 Hz, 2H), 7.26-7.16 (m, 4H), 6.96 (dt, J=1.9, 8.7 Hz, 4H), 6.84-6.64 (m, 2H), 4.03-3.68 (m, 18H), 3.67-3.51 (m, 3H), 3.42-3.34 (m, 2H), 3.29-3.24 (m, 1H), 3.23-3.06 (m, 4H), 2.93 (br s, 3H), 2.87-2.59 (m, 9H), 2.11 (br t, J=11.9 Hz, 2H), 2.02-1.89 (m, 4H), 1.38 (dd, J=4.1, 7.1 Hz, 12H), 1.20 (br d, J=6.4 Hz, 6H).

### Example 75, 1,1′-(2,2′-((3R,3′R,6R,6′R)-(((2S,2'S)-(((4-fluoro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

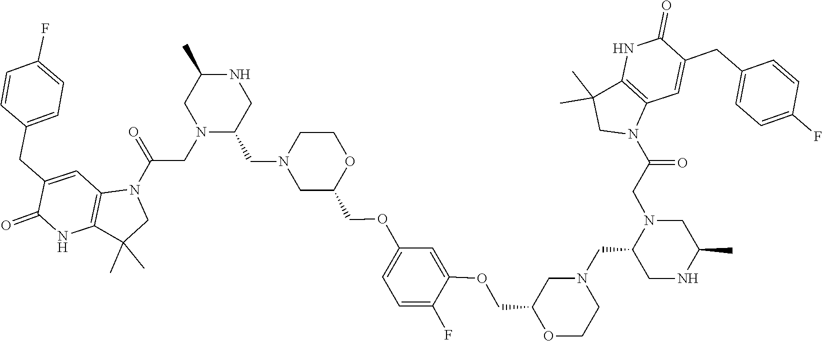 [SMILES: C[C@@H]1CN(CC(=O)N2CC(C)(C)c3[nH]c(=O)c(Cc4ccc(F)cc4)cc32)[C@@H](CN2CCO[C@H](COc3ccc(F)c(OC[C@@H]4CN(C[C@H]5CN[C@H](C)CN5CC(=O)N5CC(C)(C)c6[nH]c(=O)c(Cc7ccc(F)cc7)cc65)CCO4)c3)C2)CN1]

LCMS (ES, m/z): 588.6 [M/2+H]⁺

¹H NMR (400 MHz, METHANOL-d₄) δ=8.53 (s, 2H), 8.16 (d, J=4.1 Hz, 2H), 7.20 (br t, J=5.9 Hz, 4H), 7.03-6.89 (m, 5H), 6.52 (dd, J=2.4, 7.0 Hz, 1H), 6.38 (td, J=2.8, 5.9 Hz, 1H), 3.93-3.65 (m, 18H), 3.59 (br d, J=8.9 Hz, 2H), 3.47-3.34 (m, 4H), 3.24 (br s, 4H), 3.08-2.91 (m, 4H), 2.87-2.59 (m, 8H), 2.17 (br d, J=11.4 Hz, 2H), 2.06-1.93 (m, 4H), 1.37 (br dd, J=2.9, 7.6 Hz, 12H), 1.25 (br d, J=5.9 Hz, 6H).

### Example 76, 3,5-bis(((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholin-2-yl)methoxy)benzonitrile

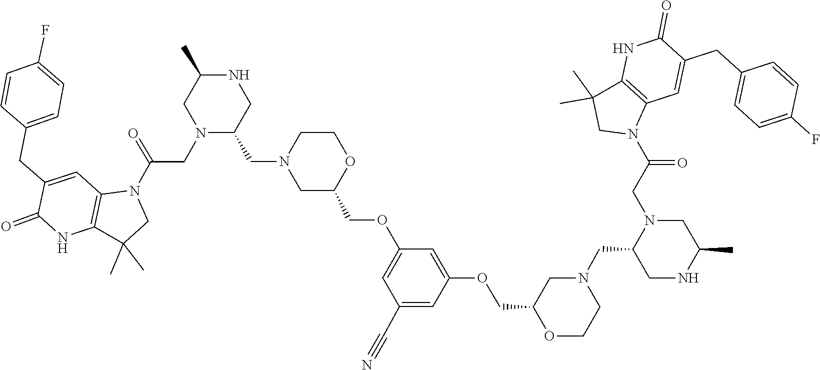 [SMILES: C[C@@H]1CN(CC(=O)N2CC(C)(C)c3[nH]c(=O)c(Cc4ccc(F)cc4)cc32)[C@@H](CN2CCO[C@H](COc3cc(C#N)cc(OC[C@@H]4CN(C[C@H]5CN[C@H](C)CN5CC(=O)N5CC(C)(C)c6[nH]c(=O)c(Cc7ccc(F)cc7)cc65)CCO4)c3)C2)CN1]

LCMS (ES, m/z): 592.0 [M/2+H]⁺

¹H NMR (400 MHz, METHANOL-d₄) δ=8.54 (s, 2H), 8.21 (s, 2H), 7.22 (dd, J=5.5, 8.4 Hz, 4H), 6.95 (t, J=8.8 Hz, 4H), 6.82 (d, J=2.1 Hz, 2H), 6.60 (s, 1H), 4.01-3.59 (m, 19H), 3.53 (br d, J=5.5 Hz, 2H), 3.42 (br t, J=10.7 Hz, 3H), 3.27-3.15 (m, 4H), 3.00-2.81 (m, 6H), 2.78-2.61 (m, 6H), 2.16 (s, 2H), 1.96 (br d, J=10.9 Hz, 4H), 1.39 (d, J=12.8 Hz, 12H), 1.24 (br d, J=6.4 Hz, 6H).

### Example 77, 2,4-bis(((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholin-2-yl)methoxy)benzonitrile

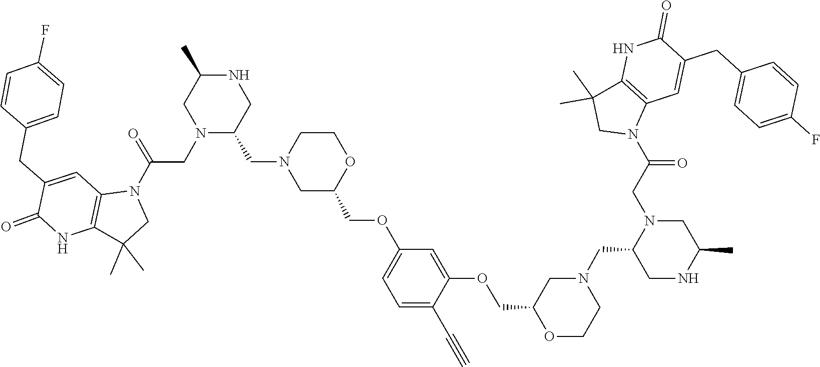 [SMILES: C#Cc1ccc(OC[C@@H]2CN(C[C@H]3CN[C@H](C)CN3CC(=O)N3CC(C)(C)c4[nH]c(=O)c(Cc5ccc(F)cc5)cc43)CCO2)cc1OC[C@@H]1CN(C[C@H]2CN[C@H](C)CN2CC(=O)N2CC(C)(C)c3[nH]c(=O)c(Cc4ccc(F)cc4)cc32)CCO1]

LCMS (ES, m/z): 592.1 [M/2+H]⁺

¹H NMR (400 MHz, METHANOL-d₄) δ=8.53 (s, 2H), 8.17 (s, 2H), 7.48 (d, J=9.1 Hz, 1H), 7.21 (dd, J=5.6, 8.3 Hz, 4H), 6.96 (t, J=8.7 Hz, 4H), 6.64-6.45 (m, 2H), 4.04-3.55 (m, 20H), 3.51-3.34 (m, 4H), 3.24 (br s, 4H), 3.13-2.93 (m, 4H), 2.92-2.59 (m, 8H), 2.25-1.89 (m, 6H), 1.48-1.32 (m, 12H), 1.25 (br d, J=6.1 Hz, 6H).

### Example 78, 1,1′-(2,2′-((3R,3′R,6R,6′R)-(((2S,2'S)—(([1,1′-biphenyl]-2,4-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

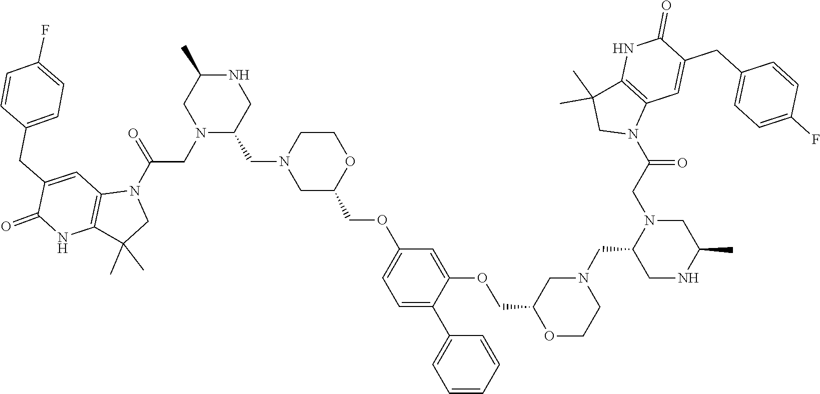 [SMILES: C[C@@H]1CN(CC(=O)N2CC(C)(C)c3[nH]c(=O)c(Cc4ccc(F)cc4)cc32)[C@@H](CN2CCO[C@H](COc3ccc(-c4ccccc4)c(OC[C@@H]4CN(C[C@H]5CN[C@H](C)CN5CC(=O)N5CC(C)(C)c6[nH]c(=O)c(Cc7ccc(F)cc7)cc65)CCO4)c3)C2)CN1]

LCMS (ES, m/z): 617.5 [M/2+H]⁺

¹H NMR (400 MHz, METHANOL-d4) δ=8.54 (s, 2H), 8.16 (d, J=10.8 Hz, 2H), 7.46-7.12 (m, 10H), 6.94 (dt, J=6.8, 8.7 Hz, 4H), 6.56-6.44 (m, 2H), 3.98-3.52 (m, 19H), 3.45-3.36 (m, 4H), 3.27-3.03 (m, 6H), 2.99-2.90 (m, 3H), 2.87-2.61 (m, 7H), 2.53 (br dd, J=8.5, 13.4 Hz, 1H), 2.20-2.09 (m, 2H), 2.05-1.87 (m, 4H), 1.37 (d, J=3.9 Hz, 6H), 1.27-1.18 (m, 9H), 1.08 (s, 3H).

### Example 79, 1,1′-(2,2′-((3R,3′R,6R,6′R)-(((2S,2'S)-(((2-methyl-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

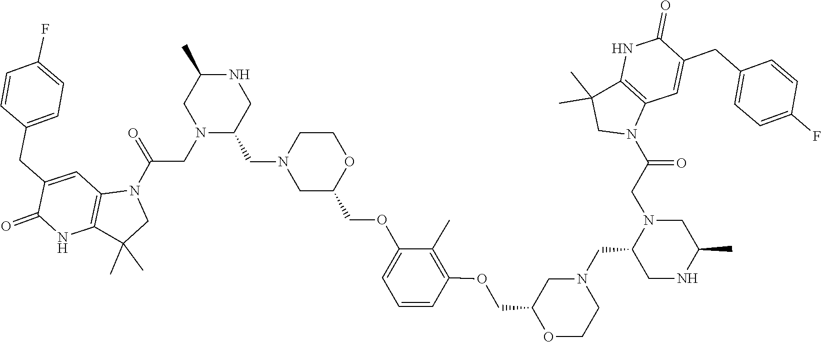 [SMILES: Cc1c(OC[C@@H]2CN(C[C@H]3CN[C@H](C)CN3CC(=O)N3CC(C)(C)c4[nH]c(=O)c(Cc5ccc(F)cc5)cc43)CCO2)cccc1OC[C@@H]1CN(C[C@H]2CN[C@H](C)CN2CC(=O)N2CC(C)(C)c3[nH]c(=O)c(Cc4ccc(F)cc4)cc32)CCO1]

LCMS (ES, m/z): 586.5 [M/2+H]⁺

¹H NMR (400 MHz, METHANOL-d4) δ=8.55 (s, 1H), 8.31-8.26 (m, 2H), 7.17 (dd, J=5.6, 8.3 Hz, 4H), 7.04-6.96 (m, 1H), 6.85 (t, J=8.7 Hz, 4H), 6.45 (d, J=8.4 Hz, 1H), 3.94-3.82 (m, 6H), 3.81-3.60 (m, 12H), 3.60-3.51 (m, 2H), 3.50-3.33 (m, 6H), 3.23-3.00 (m, 6H), 2.99-2.91 (m, 2H), 2.88-2.80 (m, 2H), 2.77-2.58 (m, 6H), 2.13 (br d, J=13.1 Hz, 2H), 2.03-1.90 (m, 4H), 1.70 (s, 2H), 1.40 (s, 5H), 1.30 (s, 6H), 1.25-1.17 (m, 6H).

### Example 80, 1,1′-(2,2′-((3R,3′R,6R,6′R)-(((2S,2'S)-(((1,3-phenylenebis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

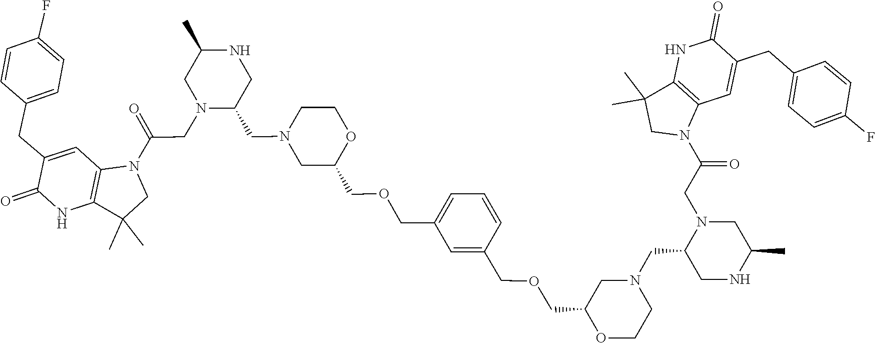 [SMILES: C[C@@H]1CN(CC(=O)N2CC(C)(C)c3[nH]c(=O)c(Cc4ccc(F)cc4)cc32)[C@@H](CN2CCO[C@H](COCc3cccc(COC[C@@H]4CN(C[C@H]5CN[C@H](C)CN5CC(=O)N5CC(C)(C)c6[nH]c(=O)c(Cc7ccc(F)cc7)cc65)CCO4)c3)C2)CN1]

LCMS (ES, m/z): 593.6 [M/2+H]⁺

¹H NMR (400 MHz, METHANOL-d4) δ=8.00 (s, 2H), 7.28-7.14 (m, 8H), 7.01 (t, J=8.6 Hz, 4H), 4.50 (s, 4H), 4.17 (br d, J=12.4 Hz, 2H), 4.05-3.89 (m, 6H), 3.87-3.50 (m, 21H), 3.49-3.37 (m, 4H), 3.26 (br d, J=8.6 Hz, 7H), 3.16-3.02 (m, 6H), 1.44 (d, J=7.4 Hz, 12H), 1.32 (d, J=6.4 Hz, 6H).

### Example 81, 1,1′-(2,2′-((3R,3′R,6R,6′R)-(((2S,2'S)-((butane-1,4-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

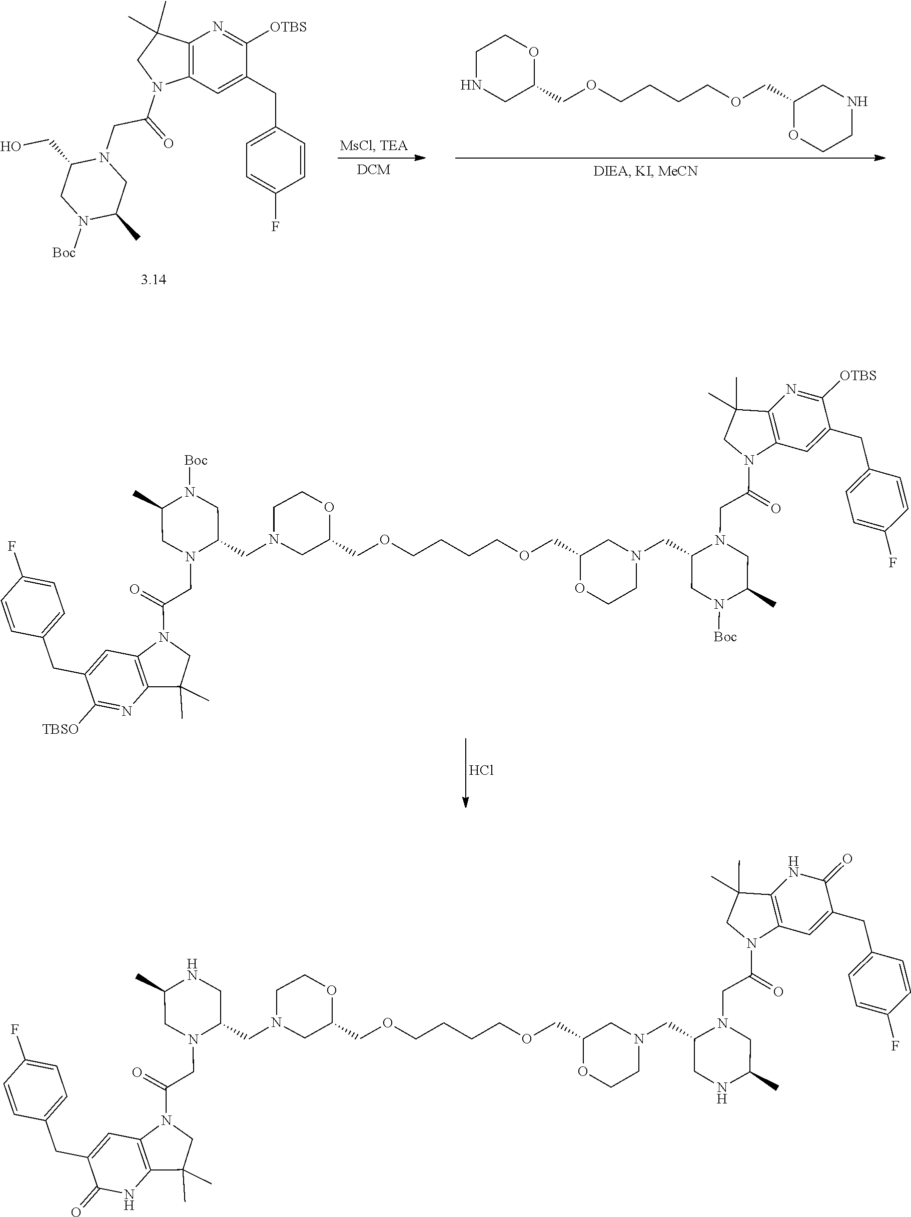

LCMS (ES, m/z): 569.5 [M/2+H]⁺

¹H NMR (400 MHz, METHANOL-d4) δ=8.02-7.97 (m, 2H), 7.28-7.19 (m, 4H), 7.05 (t, J=8.8 Hz, 4H), 4.17-4.06 (m, 2H), 4.01-3.93 (m, 2H), 3.90-3.84 (m, 2H), 3.82-3.64 (m, 14H), 3.54-3.38 (m, 12H), 3.29-3.18 (m, 8H), 3.17-3.06 (m, 4H), 3.05-2.85 (m, 6H), 1.59-1.48 (m, 4H), 1.44-1.37 (m, 12H), 1.34-1.25 (m, 6H).

### Example 82, 1,1′-(2,2′-((3R,3′R,6R,6′R)-(((2S,2'S)-((pentane-1,5-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

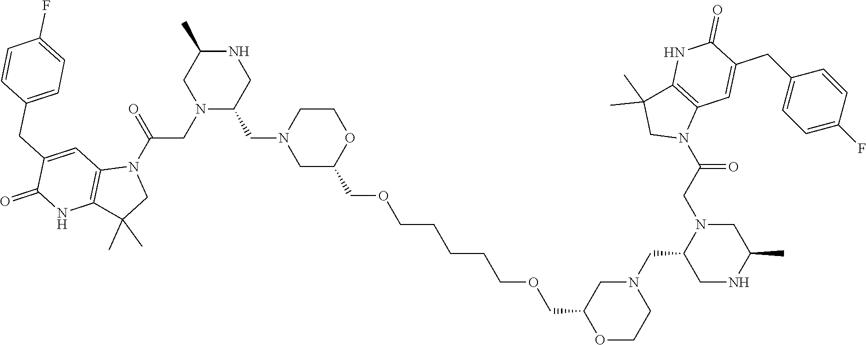 [SMILES: C[C@@H]1CN(CC(=O)N2CC(C)(C)c3[nH]c(=O)c(Cc4ccc(F)cc4)cc32)[C@@H](CN2CCO[C@H](COCCCCCOC[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 576.4 [M/2+H]⁺

¹H NMR (400 MHz, METHANOL-d4) δ=8.06-8.00 (m, 2H), 7.30-7.20 (m, 4H), 7.11-7.01 (m, 4H), 4.14-4.03 (m, 2H), 4.01-3.93 (m, 2H), 3.87 (br d, J=10.8 Hz, 2H), 3.82-3.63 (m, 14H), 3.54-3.43 (m, 8H), 3.41-3.36 (m, 4H), 3.28-3.18 (m, 9H), 3.12-3.00 (m, 6H), 2.93-2.80 (m, 3H), 1.52-1.39 (m, 16H), 1.33-1.25 (m, 8H).

### Example 83, 1,1′-(2,2′-((3R,3′R,6R,6′R)-(((2S,2'S)-(((oxybis(ethane-2,1-diyl))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

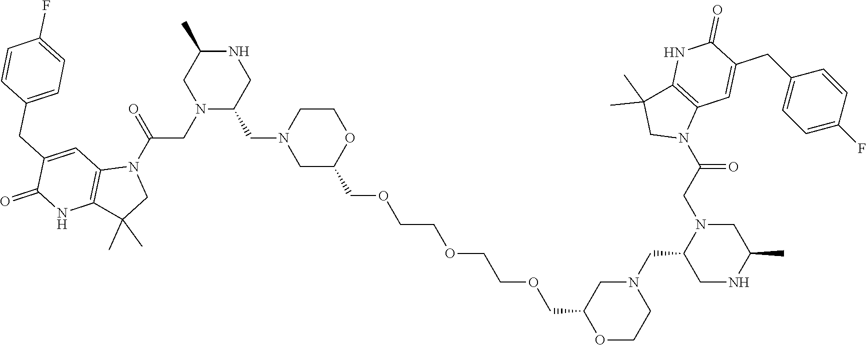 [SMILES: C[C@@H]1CN(CC(=O)N2CC(C)(C)c3[nH]c(=O)c(Cc4ccc(F)cc4)cc32)[C@@H](CN2CCO[C@H](COCCOCCOC[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 577.4 [M/2+H]⁺

¹H NMR (400 MHz, METHANOL-d4) δ=7.99 (s, 2H), 7.24 (dd, J=5.4, 8.4 Hz, 4H), 7.12-7.02 (m, 4H), 4.09 (br d, J=12.1 Hz, 2H), 4.01-3.94 (m, 2H), 3.92-3.82 (m, 3H), 3.81-3.78 (m, 7H), 3.76-3.68 (m, 5H), 3.64 (br d, J=13.5 Hz, 2H), 3.59-3.47 (m, 15H), 3.23 (br t, J=11.9 Hz, 8H), 3.15-3.06 (m, 4H), 3.05-2.98 (m, 2H), 2.90 (br s, 4H), 1.42 (d, J=7.6 Hz, 12H), 1.30 (d, J=6.5 Hz, 6H).

### Example 84, 1,1′-(2,2′-((3R,3′R,6R,6′R)-(((2S,2'S)-(((pyridine-2,6-diylbis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

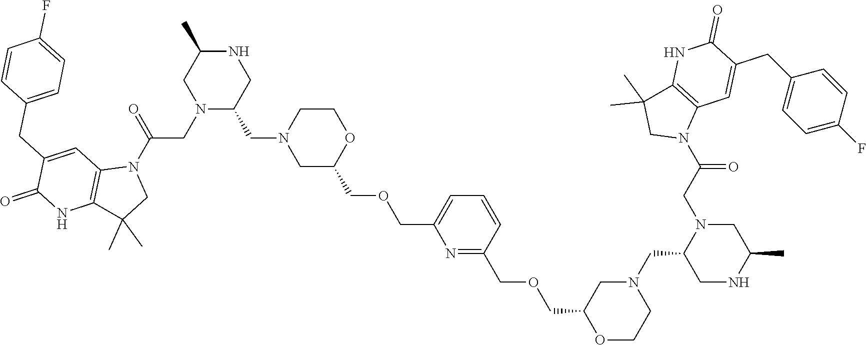 [SMILES: C[C@@H]1CN(CC(=O)N2CC(C)(C)c3[nH]c(=O)c(Cc4ccc(F)cc4)cc32)[C@@H](CN2CCO[C@H](COCc3cccc(COC[C@@H]4CN(C[C@H]5CN[C@H](C)CN5CC(=O)N5CC(C)(C)c6[nH]c(=O)c(Cc7ccc(F)cc7)cc65)CCO4)n3)C2)CN1]

LCMS (ES, m/z): 594.0 [M/2+H]⁺

¹H NMR (400 MHz, METHANOL-d4) δ=8.08-7.97 (m, 2H), 7.70-7.60 (m, 1H), 7.28 (br d, J=7.9 Hz, 6H), 6.99 (br t, J=8.6 Hz, 4H), 4.64-4.56 (m, 4H), 4.17-4.04 (m, 2H), 3.98-3.86 (m, 4H), 3.84-3.57 (m, 20H), 3.53-3.40 (m, 4H), 3.28-3.16 (m, 8H), 3.13-3.02 (m, 6H), 2.95-2.86 (m, 2H), 1.44-1.37 (m, 12H), 1.31-1.26 (m, 6H).

### Example 85, 1,1′-(2,2′-((3R,3′R,6R,6′R)-(((2S,2'S)-((((perfluoro-1,4-phenylene)bis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

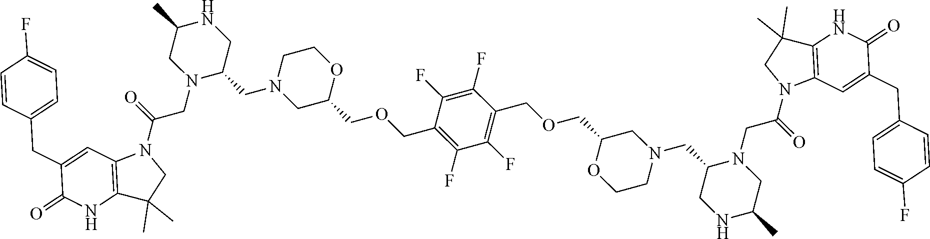 [SMILES: C[C@@H]1CN(CC(=O)N2CC(C)(C)c3[nH]c(=O)c(Cc4ccc(F)cc4)cc32)[C@@H](CN2CCO[C@H](COCc3c(F)c(F)c(COC[C@@H]4CN(C[C@H]5CN[C@H](C)CN5CC(=O)N5CC(C)(C)c6[nH]c(=O)c(Cc7ccc(F)cc7)cc65)CCO4)c(F)c3F)C2)CN1]

LCMS (ES, m/z): 629.5 [M/2+H]⁺

¹H NMR (400 MHz, METHANOL-d4) δ=8.03-7.94 (m, 2H), 7.23-7.15 (m, 4H), 7.05-6.95 (m, 4H), 4.65 (br s, 4H), 4.09-4.00 (m, 2H), 3.97-3.91 (m, 2H), 3.86-3.83 (m, 2H), 3.80-3.70 (m, 10H), 3.67-3.53 (m, 8H), 3.52-3.42 (m, 4H), 3.23-3.11 (m, 8H), 3.10-2.99 (m, 6H), 2.89-2.71 (m, 4H), 1.44-1.37 (m, 12H), 1.30 (d, J=6.5 Hz, 6H).

### Example 86, 1,1′-(2,2′-((3R,3′R,6R,6′R)-(((2S,2'S)-(((5-methyl-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

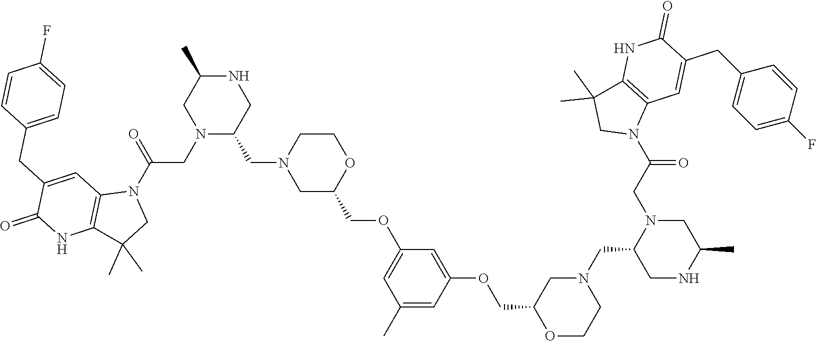 [SMILES: Cc1cc(OC[C@@H]2CN(C[C@H]3CN[C@H](C)CN3CC(=O)N3CC(C)(C)c4[nH]c(=O)c(Cc5ccc(F)cc5)cc43)CCO2)cc(OC[C@@H]2CN(C[C@H]3CN[C@H](C)CN3CC(=O)N3CC(C)(C)c4[nH]c(=O)c(Cc5ccc(F)cc5)cc43)CCO2)c1]

L CMS (ES, m/z): 586.5 [M/2+H]⁺

¹H NMR (400 MHz, METHANOL-d₄) δ=8.07-7.99 (m, 2H), 7.20-7.08 (m, 4H), 6.97 (br t, J=8.6 Hz, 4H), 6.41-6.31 (m, 2H), 6.24-6.18 (m, 1H), 4.16-3.86 (m, 12H), 3.85-3.58 (m, 14H), 3.54-3.40 (m, 4H), 3.29-3.13 (m, 8H), 3.12-2.73 (m, 10H), 2.28-2.21 (m, 3H), 1.47-1.34 (m, 12H), 1.30 (br d, J=6.4 Hz, 6H).

### Example 87, 1,1′-(2,2′-((3R,3′R,6R,6′R)-(((2S,2'S)-(((2-bromo-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

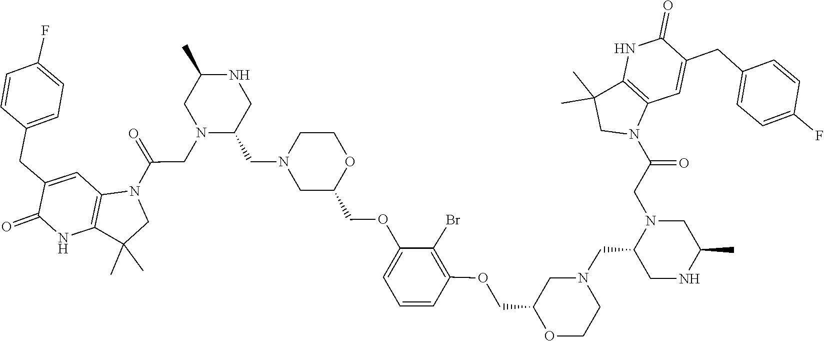 [SMILES: C[C@@H]1CN(CC(=O)N2CC(C)(C)c3[nH]c(=O)c(Cc4ccc(F)cc4)cc32)[C@@H](CN2CCO[C@H](COc3cccc(OC[C@@H]4CN(C[C@H]5CN[C@H](C)CN5CC(=O)N5CC(C)(C)c6[nH]c(=O)c(Cc7ccc(F)cc7)cc65)CCO4)c3Br)C2)CN1]

LCMS (ES, m/z): 618.3, 620.3 [M/2+H]⁺

¹H NMR (400 MHz, METHANOL-d₄) δ=8.32-8.19 (m, 2H), 7.30-7.12 (m, 5H), 6.99-6.76 (m, 4H), 6.74-6.61 (m, 2H), 4.21-3.74 (m, 22H), 3.73-3.54 (m, 4H), 3.50-3.34 (m, 4H), 3.26-2.71 (m, 16H), 1.51-1.43 (m, 6H), 1.33-1.22 (m, 12H).

### Example 88, 1,1′-(2,2′-((3R,3′R,6R,6′R)-(((2S,2'S)-(((2-chloro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

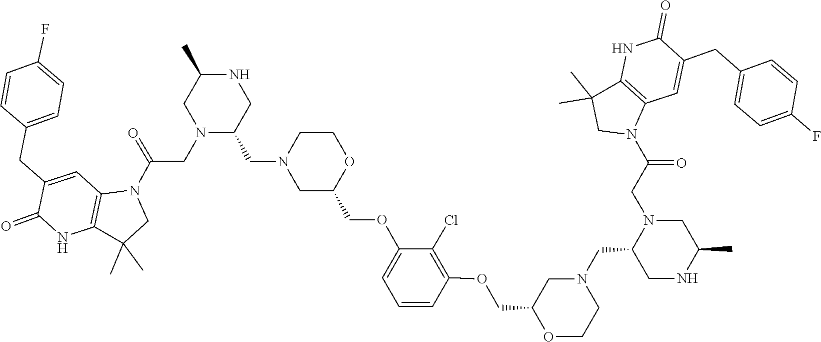 [SMILES: C[C@@H]1CN(CC(=O)N2CC(C)(C)c3[nH]c(=O)c(Cc4ccc(F)cc4)cc32)[C@@H](CN2CCO[C@H](COc3cccc(OC[C@@H]4CN(C[C@H]5CN[C@H](C)CN5CC(=O)N5CC(C)(C)c6[nH]c(=O)c(Cc7ccc(F)cc7)cc65)CCO4)c3Cl)C2)CN1]

LCMS (ES, m/z): 596.4 [M/2+H]⁺

¹H NMR (400 MHz, METHANOL-d₄) δ=8.25-8.16 (m, 2H), 7.26-7.14 (m, 5H), 6.96-6.86 (m, 4H), 6.76-6.69 (m, 2H), 4.10-3.96 (m, 6H), 3.95-3.46 (m, 22H), 3.45-3.33 (m, 4H), 3.26-3.07 (m, 10H), 3.07-2.90 (m, 4H), 1.50-1.44 (m, 6H), 1.33-1.27 (m, 12H).

### Example 89, 1,1′-(2,2′-((3R,3′R,6R,6′R)-(((2S,2'S)-(((4-chloro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

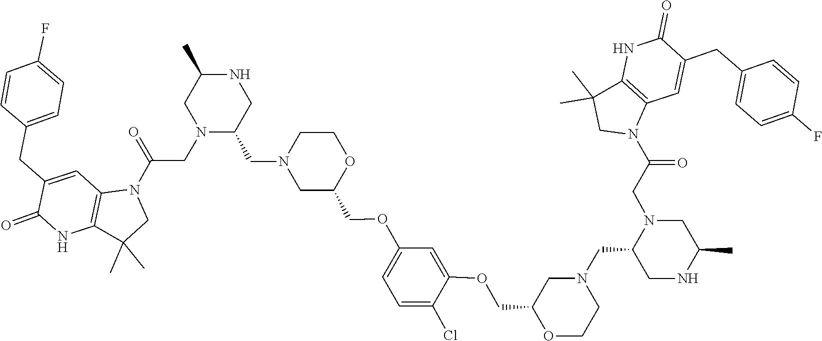 [SMILES: C[C@@H]1CN(CC(=O)N2CC(C)(C)c3[nH]c(=O)c(Cc4ccc(F)cc4)cc32)[C@@H](CN2CCO[C@H](COc3ccc(Cl)c(OC[C@@H]4CN(C[C@H]5CN[C@H](C)CN5CC(=O)N5CC(C)(C)c6[nH]c(=O)c(Cc7ccc(F)cc7)cc65)CCO4)c3)C2)CN1]

LCMS (ES, m/z): 596.4 [M/2+H]⁺

¹H NMR (400 MHz, METHANOL-d₄) δ=8.03 (d, J=8.0 Hz, 2H), 7.18 (ddd, J=5.2, 8.9, 14.1 Hz, 5H), 6.98 (q, J=9.0 Hz, 4H), 6.63 (s, 1H), 6.53 (dd, J=2.6, 8.8 Hz, 1H), 4.05 (br s, 6H), 3.98-3.83 (m, 7H), 3.80 (br d, J=4.5 Hz, 4H), 3.77-3.60 (m, 8H), 3.45 (br s, 2H), 3.27 (br d, J=12.6 Hz, 4H), 3.22-3.11 (m, 4H), 3.10-2.97 (m, 5H), 2.96-2.61 (m, 6H), 1.47-1.35 (m, 12H), 1.30 (d, J=6.4 Hz, 6H).

### Example 90, (2S,2'S)-N,N′-(hexane-1,6-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

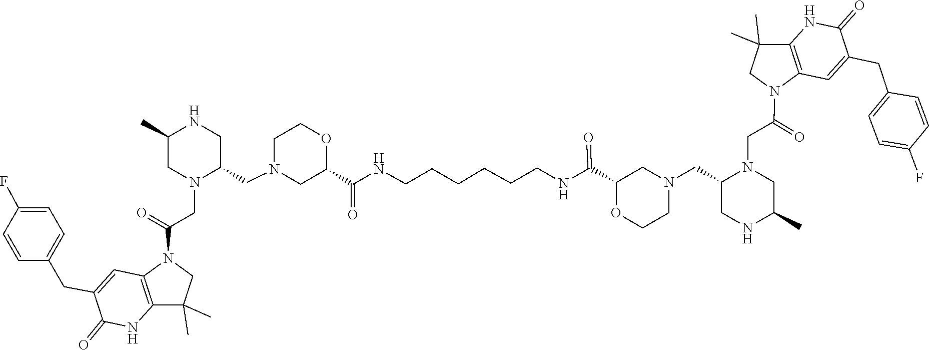

LCMS (ES, m/z): 1191.5 [M+H]⁺

¹H NMR (400 MHz, MeOD) ppm δ 8.53 (s, 1H), 8.18 (s, 2H), 7.27-7.22 (m, 4H), 7.03-6.96 (m, 4H), 3.88-3.82 (m, 8H), 3.71-3.62 (m, 4H), 3.54-3.42 (m, 2H), 3.27-3.11 (m, 8H), 3.09-3.00 (m, 6H), 2.98-2.92 (m, 2H), 2.83-2.77 (d, J=12 Hz, 2H), 2.70-2.58 (m, 4H), 2.20-2.14 (m, 2H), 2.01-1.87 (m, 4H), 1.43-1.36 (m, 14H), 1.34-1.23 (m, 8H), 1.22-1.19 (d, J=8 Hz, 6H).

### Example 91, (2S,2'S)-N,N′-(1,4-phenylenebis(methylene))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

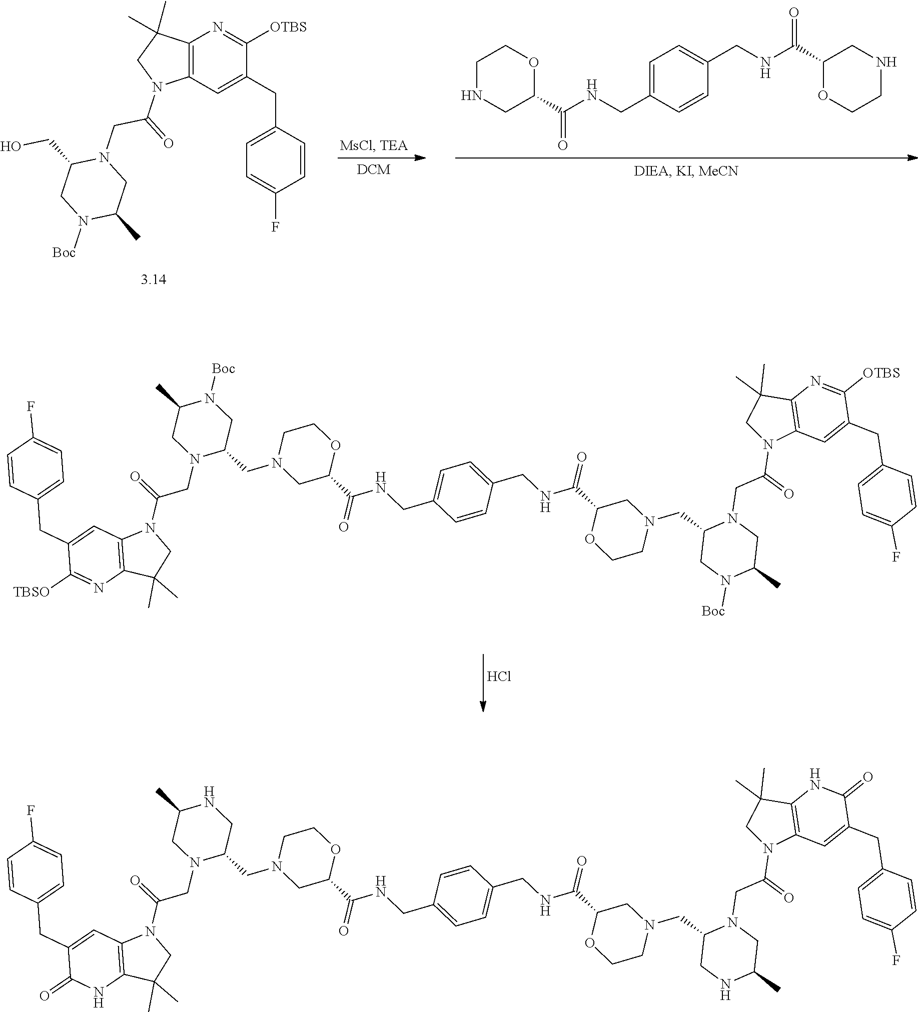

LCMS (ES, m/z): 606.4 [M/2+H]⁺

¹H NMR (400 MHz, METHANOL-d₄) δ=8.44 (s, 2H), 8.19 (s, 2H), 7.28-7.21 (m, 4H), 7.14 (s, 4H), 7.02-6.93 (m, 4H), 4.44 (d, J=15.0 Hz, 2H), 4.21 (d, J=15.0 Hz, 2H), 3.94-3.52 (m, 20H), 3.35 (s, 1H), 3.30-3.20 (m, 5H), 3.17-2.97 (m, 6H), 2.84-2.61 (m, 6H), 2.20 (dd, J=2.6, 13.2 Hz, 2H), 2.04-1.94 (m, 4H), 1.37 (d, J=13.9 Hz, 12H), 1.27 (d, J=6.4 Hz, 6H).

### Example 92, (2S,2'S)-N,N′-((1S,4S)-cyclohexane-1,4-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

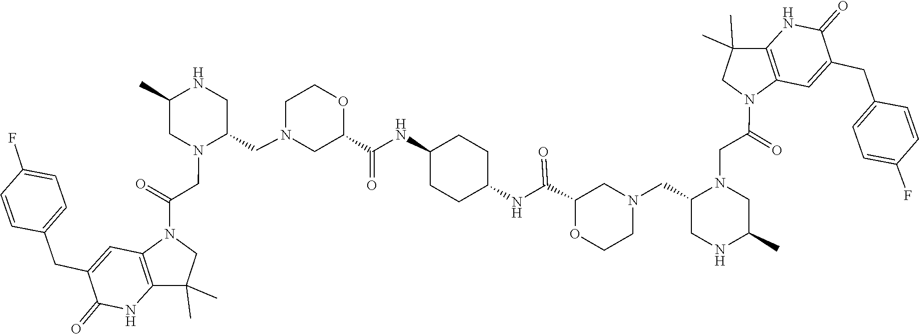 [SMILES: C[C@@H]1CN(CC(=O)N2CC(C)(C)c3[nH]c(=O)c(Cc4ccc(F)cc4)cc32)[C@@H](CN2CCO[C@H](C(=O)N[C@H]3CC[C@H](NC(=O)[C@@H]4CN(C[C@H]5CN[C@H](C)CN5CC(=O)N5CC(C)(C)c6[nH]c(=O)c(Cc7ccc(F)cc7)cc65)CCO4)CC3)C2)CN1]

LCMS (ES, m/z): 1189.5 [M/2+H]⁺

¹H NMR (400 MHz, DMSO) δ=8.32 (s, 2H), 8.06 (s, 2H), 7.41 (d, J=8.4, 2H), 7.29-7.21 (m, 4H), 7.16-7.04 (m, 4H), 3.84 (d, J=10.5, 2H), 3.80-3.67 (m, 8H), 3.66-3.53 (m, 6H), 3.47 (s, 2H), 3.34 (s, 2H), 3.21 (t, J=10.6, 2H), 3.09-2.91 (m, 6H), 2.90-2.75 (m, 4H), 2.70 (d, J=10.8, 2H), 2.59-2.52 (m, 2H), 2.50-2.40 (m, 4H), 2.16-2.02 (m, 2H), 1.90 (t, J=10.0, 2H), 1.79 (t, J=10.7, 2H), 1.59 (s, 4H), 1.35-1.30 (s, 2H), 1.28 (d, J=6.0, 12H), 1.07 (d, J=6.1, 6H).

### Example 93, (2S,2'S)-N,N′-(hexa-2,4-diyne-1,6-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

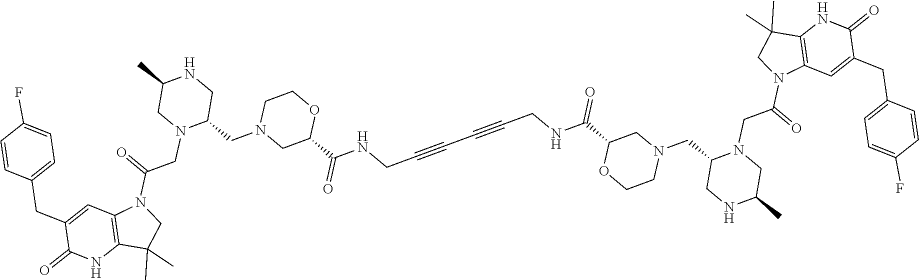 [SMILES: C[C@@H]1CN(CC(=O)N2CC(C)(C)c3[nH]c(=O)c(Cc4ccc(F)cc4)cc32)[C@@H](CN2CCO[C@H](C(=O)NCC#CC#CCNC(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 592.5 [M/2+H]⁺

¹H NMR (400 MHz, METHANOL-d₄) δ=8.46-8.37 (m, 1H), 8.11-8.04 (m, 2H), 7.28-7.19 (m, 4H), 7.07-6.96 (m, 4H), 4.17-4.06 (m, 2H), 4.05-3.93 (m, 4H), 3.93-3.71 (m, 14H), 3.70-3.61 (m, 2H), 3.58-3.46 (m, 2H), 3.45-3.33 (m, 4H), 3.29-3.20 (m, 4H), 3.19-3.02 (m, 6H), 3.00-2.83 (m, 4H), 2.74-2.58 (m, 2H), 2.54-2.33 (m, 4H), 1.46-1.34 (m, 12H), 1.32-1.23 (m, 6H).

### Example 94, 1,1′-(2,2′-((3R,3′R,6R,6′R)-((propane-1,3-diylbis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

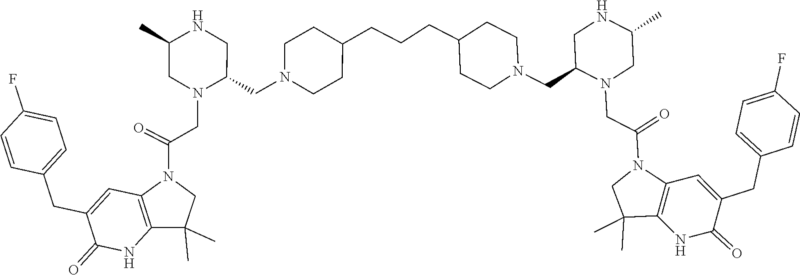 [SMILES: C[C@@H]1CN(CC(=O)N2CC(C)(C)c3[nH]c(=O)c(Cc4ccc(F)cc4)cc32)[C@@H](CN2CCC(CCCC3CCN(C[C@H]4CN[C@H](C)CN4CC(=O)N4CC(C)(C)c5[nH]c(=O)c(Cc6ccc(F)cc6)cc54)CC3)CC2)CN1]

LCMS (ES, m/z): 530.5 [M/2+H]⁺

¹H NMR (400 MHz, METHANOL-d₄) δ=8.43 (s, 2H), 8.23 (s, 2H), 7.23 (dd, J=5.5, 8.4 Hz, 4H), 6.97 (t, J=8.8 Hz, 4H), 3.90-3.71 (m, 10H), 3.68-3.50 (m, 4H), 3.27-3.12 (m, 6H), 3.02 (br t, J=13.5 Hz, 4H), 2.81 (br d, J=11.0 Hz, 2H), 2.76-2.60 (m, 4H), 2.20 (br d, J=12.9 Hz, 2H), 2.14-1.97 (m, 2H), 1.81 (br t, J=10.8 Hz, 2H), 1.55 (br d, J=12.0 Hz, 2H), 1.45 (br s, 2H), 1.38 (d, J=7.4 Hz, 12H), 1.26 (br d, J=6.1 Hz, 6H), 1.18-0.71 (m, 12H).

### Example 95, N¹,N³-bis((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)-N¹,N³-dimethylisophthalamide

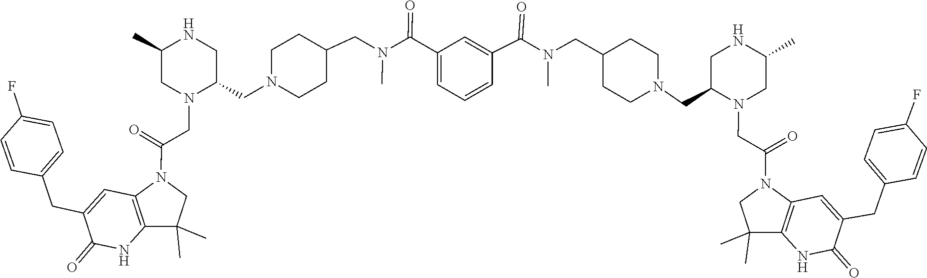 [SMILES: C[C@@H]1CN(CC(=O)N2CC(C)(C)c3[nH]c(=O)c(Cc4ccc(F)cc4)cc32)[C@@H](CN2CCC(CN(C)C(=O)c3cccc(C(=O)N(C)CC4CCN(C[C@H]5CN[C@H](C)CN5CC(=O)N5CC(C)(C)c6[nH]c(=O)c(Cc7ccc(F)cc7)cc65)CC4)c3)CC2)CN1]

LCMS (ES, m/z): 618.6 [M/2+H]⁺

¹H NMR (400 MHz, METHANOL-d₄) δ=8.43 (br s, 3H), 8.26-8.08 (m, 2H), 7.57-7.32 (m, 3H), 7.25 (br s, 4H), 7.15-6.94 (m, 4H), 3.91-3.59 (m, 11H), 3.48 (br s, 2H), 3.30-2.91 (m, 17H), 2.91-2.79 (m, 5H), 2.78-2.50 (m, 5H), 2.19 (br s, 1H), 2.13-1.99 (m, 2H), 1.89 (br d, J=10.8 Hz, 2H), 1.77 (br s, 2H), 1.69-1.51 (m, 3H), 1.42 (br d, J=13.3 Hz, 9H), 1.26 (br d, J=6.3 Hz, 10H), 1.12 (br s, 3H), 0.94-0.52 (m, 2H).

### Example 96, N¹,N⁴-bis((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)-N¹,N⁴-dimethylterephthalamide

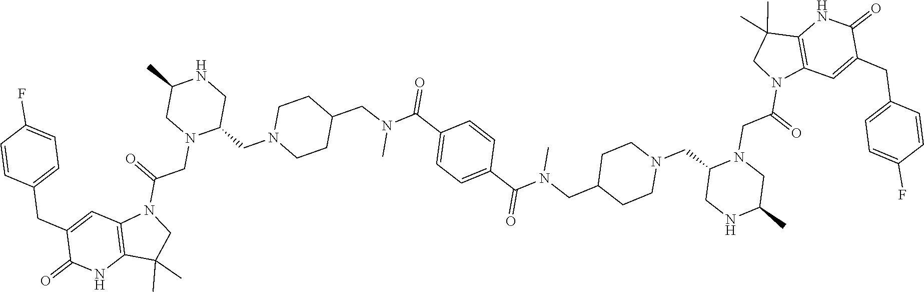 [SMILES: C[C@@H]1CN(CC(=O)N2CC(C)(C)c3[nH]c(=O)c(Cc4ccc(F)cc4)cc32)[C@@H](CN2CCC(CN(C)C(=O)c3ccc(C(=O)N(C)CC4CCN(C[C@H]5CN[C@H](C)CN5CC(=O)N5CC(C)(C)c6[nH]c(=O)c(Cc7ccc(F)cc7)cc65)CC4)cc3)CC2)CN1]

LCMS (ES, m/z): 618.6 [M/2+H]⁺

¹H NMR (400 MHz, METHANOL-d₄) δ=8.43 (br s, 3H), 8.26-8.09 (m, 2H), 7.43 (br s, 2H), 7.34-7.23 (m, 5H), 7.09-6.93 (m, 4H), 3.92-3.62 (m, 10H), 3.61-3.33 (m, 5H), 3.29-3.10 (m, 6H), 3.10-2.84 (m, 14H), 2.83-2.55 (m, 5H), 2.41-2.11 (m, 2H), 2.03 (br s, 2H), 1.94-1.77 (m, 2H), 1.76-1.51 (m, 4H), 1.50-1.35 (m, 8H), 1.34-1.20 (m, 9H), 1.20-1.09 (m, 5H), 0.83-0.41 (m, 2H).

### Example 97, N-((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)-4-(2-(((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)(methyl)amino)-2-oxoethyl)-N-methylbenzamide

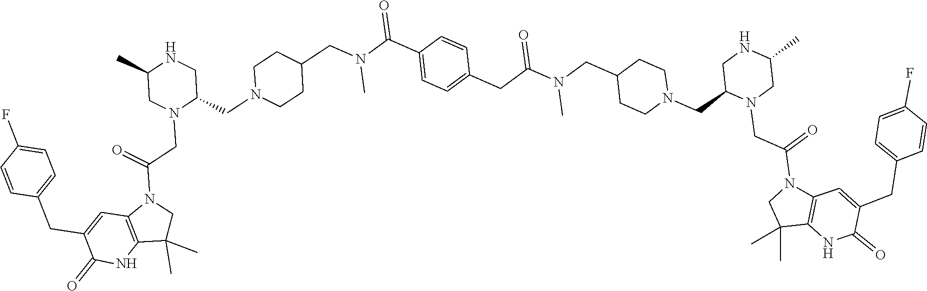 [SMILES: C[C@@H]1CN(CC(=O)N2CC(C)(C)c3[nH]c(=O)c(Cc4ccc(F)cc4)cc32)[C@@H](CN2CCC(CN(C)C(=O)Cc3ccc(C(=O)N(C)CC4CCN(C[C@H]5CN[C@H](C)CN5CC(=O)N5CC(C)(C)c6[nH]c(=O)c(Cc7ccc(F)cc7)cc65)CC4)cc3)CC2)CN1]

LCMS (ES, m/z): 625.5 [M/2+H]⁺

¹H NMR (400 MHz, METHANOL-d₄) δ=8.21 (br s, 1H), 7.97 (br s, 1H), 7.37 (br s, 4H), 7.24 (br s, 4H), 6.98 (br s, 4H), 4.05-3.96 (m, 2H), 3.95-3.65 (m, 16H), 3.63-3.33 (m, 11H), 3.28-2.81 (m, 20H), 2.30-1.79 (m, 5H), 1.78-1.51 (m, 4H), 1.42 (br d, J=5.5 Hz, 12H), 1.31 (br d, J=6.3 Hz, 6H).

### Example 98, N-((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)-3-(2-(((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)(methyl)amino)-2-oxoethyl)-N-methylbenzamide

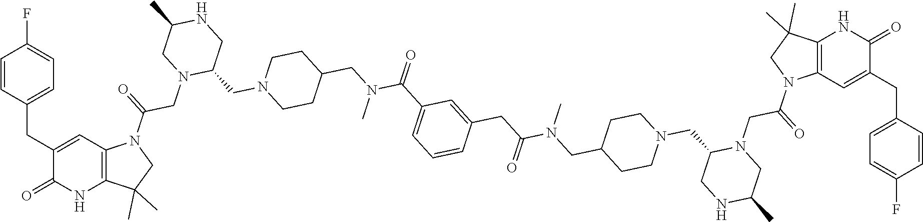 [SMILES: C[C@@H]1CN(CC(=O)N2CC(C)(C)c3[nH]c(=O)c(Cc4ccc(F)cc4)cc32)[C@@H](CN2CCC(CN(C)C(=O)Cc3cccc(C(=O)N(C)CC4CCN(C[C@H]5CN[C@H](C)CN5CC(=O)N5CC(C)(C)c6[nH]c(=O)c(Cc7ccc(F)cc7)cc65)CC4)c3)CC2)CN1]

LCMS (ES, m/z): 625.6 [M/2+H]⁺

¹H NMR (400 MHz, METHANOL-d₄) δ=8.39 (br s, 3H), 8.34-8.09 (m, 2H), 7.42-7.12 (m, 6H), 7.07-6.94 (m, 4H), 6.92-6.53 (m, 1H), 4.23-3.35 (m, 17H), 3.28-2.48 (m, 26H), 2.30-1.71 (m, 4H), 1.70-1.34 (m, 16H), 1.33-1.18 (m, 8H), 1.17-0.43 (m, 5H).

### Example 99, 2,2′-(1,4-phenylene)bis(N-((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)-N-methylacetamide)

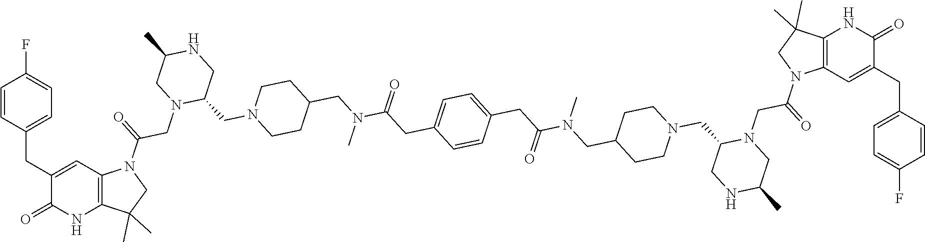 [SMILES: C[C@@H]1CN(CC(=O)N2CC(C)(C)c3[nH]c(=O)c(Cc4ccc(F)cc4)cc32)[C@@H](CN2CCC(CN(C)C(=O)Cc3ccc(CC(=O)N(C)CC4CCN(C[C@H]5CN[C@H](C)CN5CC(=O)N5CC(C)(C)c6[nH]c(=O)c(Cc7ccc(F)cc7)cc65)CC4)cc3)CC2)CN1]

LCMS (ES, m/z): 632.7 [M/2+H]⁺

¹H NMR (400 MHz, METHANOL-d₄) δ=8.45 (br s, 2H), 8.39 (s, 1H), 8.21 (s, 1H), 7.33 (dd, J=5.5, 8.3 Hz, 2H), 7.29-7.21 (m, 2H), 7.21-7.12 (m, 2H), 7.03-6.93 (m, 4H), 6.83 (s, 2H), 4.10 (br d, J=17.6 Hz, 1H), 3.96-3.58 (m, 13H), 3.57-3.35 (m, 4H), 3.28-3.12 (m, 6H), 3.09 (br d, J=11.1 Hz, 2H), 3.05-2.84 (m, 9H), 2.83-2.79 (m, 4H), 2.79-2.55 (m, 6H), 2.18-1.89 (m, 4H), 1.83-1.55 (m, 5H), 1.53-1.34 (m, 14H), 1.32-1.19 (m, 6H), 1.10-0.76 (m, 4H).

### Example 100, 1,1′-(2,2′-((3R,3′R,6R,6′R)-(((((1,4-phenylenebis(methylene))bis(methylazanediyl))bis(methylene))bis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

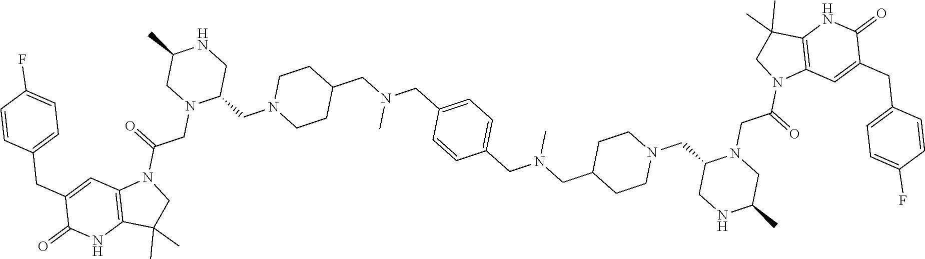 [SMILES: C[C@@H]1CN(CC(=O)N2CC(C)(C)c3[nH]c(=O)c(Cc4ccc(F)cc4)cc32)[C@@H](CN2CCC(CN(C)Cc3ccc(CN(C)CC4CCN(C[C@H]5CN[C@H](C)CN5CC(=O)N5CC(C)(C)c6[nH]c(=O)c(Cc7ccc(F)cc7)cc65)CC4)cc3)CC2)CN1]

LCMS (ES, m/z): 604.6 [M/2+H]⁺

¹H NMR (400 MHz, METHANOL-d₄) δ=8.42 (s, 4H), 8.25 (s, 2H), 7.33-7.26 (m, 6H), 7.07-6.95 (m, 4H), 4.03-3.75 (m, 10H), 3.73-3.57 (m, 7H), 3.30-3.14 (m, 6H), 3.13-2.91 (m, 5H), 2.90-2.58 (m, 6H), 2.40 (br s, 7H), 2.25-2.01 (m, 7H), 1.93-1.52 (m, 8H), 1.39 (br d, J=13.3 Hz, 12H), 1.27 (br d, J=5.4 Hz, 6H), 1.12-0.66 (m, 4H).

### Example 101, N¹,N⁴-bis((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)terephthalamide

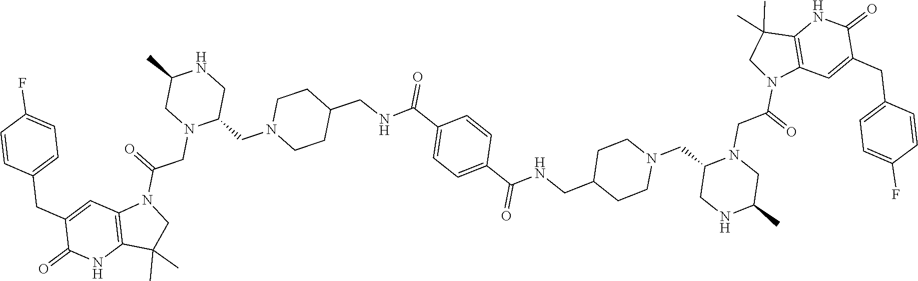 [SMILES: C[C@@H]1CN(CC(=O)N2CC(C)(C)c3[nH]c(=O)c(Cc4ccc(F)cc4)cc32)[C@@H](CN2CCC(CNC(=O)c3ccc(C(=O)NCC4CCN(C[C@H]5CN[C@H](C)CN5CC(=O)N5CC(C)(C)c6[nH]c(=O)c(Cc7ccc(F)cc7)cc65)CC4)cc3)CC2)CN1]

LCMS (ES, m/z): 604.6 [M/2+H]⁺

¹H NMR (400 MHz, METHANOL-d₄) δ=8.45 (s, 2H), 8.15 (s, 2H), 7.81 (s, 4H), 7.23 (dd, J=5.6, 8.4 Hz, 4H), 6.97 (t, J=8.8 Hz, 4H), 3.85 (s, 1H), 3.80 (dd, J=4.3, 9.6 Hz, 6H), 3.76 (s, 1H), 3.72 (s, 4H), 3.40 (br s, 2H), 3.27-3.19 (m, 4H), 3.18-3.11 (m, 2H), 3.10-2.95 (m, 8H), 2.83 (br d, J=11.8 Hz, 2H), 2.72 (br t, J=11.9 Hz, 2H), 2.64 (br dd, J=8.0, 12.8 Hz, 2H), 2.16 (br d, J=12.4 Hz, 2H), 2.02 (br t, J=11.1 Hz, 2H), 1.89 (br t, J=11.0 Hz, 2H), 1.74-1.51 (m, 6H), 1.41 (d, J=6.1 Hz, 12H), 1.25 (d, J=6.4 Hz, 6H), 1.18-1.04 (m, 4H).

### Example 102, 2,2′-(1,4-phenylene)bis(N-((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)acetamide)

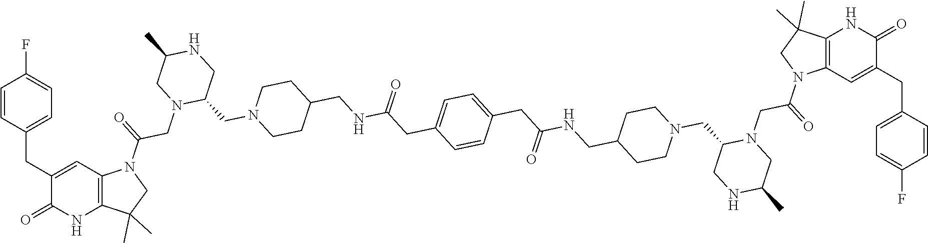 [SMILES: C[C@@H]1CN(CC(=O)N2CC(C)(C)c3[nH]c(=O)c(Cc4ccc(F)cc4)cc32)[C@@H](CN2CCC(CNC(=O)Cc3ccc(CC(=O)NCC4CCN(C[C@H]5CN[C@H](C)CN5CC(=O)N5CC(C)(C)c6[nH]c(=O)c(Cc7ccc(F)cc7)cc65)CC4)cc3)CC2)CN1]

LCMS (ES, m/z): 618.7 [M/2+H]⁺

¹H NMR (400 MHz, METHANOL-d₄) δ=8.40 (s, 3H), 8.16 (s, 2H), 7.25-7.19 (m, 7H), 6.98 (t, J=8.8 Hz, 4H), 4.01-3.74 (m, 9H), 3.72 (br s, 3H), 3.53-3.38 (m, 6H), 3.22 (br d, J=11.9 Hz, 4H), 3.02 (br d, J=6.6 Hz, 6H), 2.97-2.62 (m, 10H), 2.37-1.83 (m, 6H), 1.75-1.45 (m, 6H), 1.40 (br d, J=10.0 Hz, 12H), 1.26 (br d, J=6.1 Hz, 6H), 1.05 (br d, J=9.9 Hz, 4H).

### Example 103, N¹,N⁴-bis(2-(1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)ethyl)terephthalamide

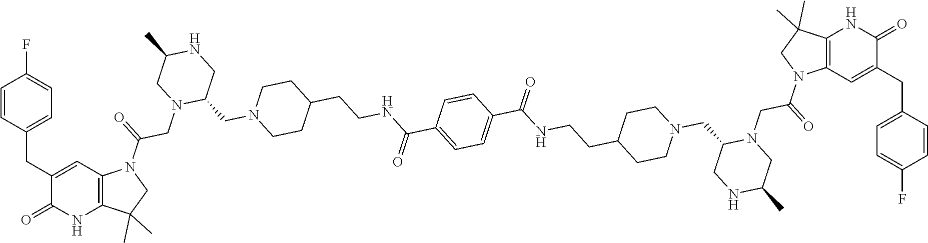 [SMILES: C[C@@H]1CN(CC(=O)N2CC(C)(C)c3[nH]c(=O)c(Cc4ccc(F)cc4)cc32)[C@@H](CN2CCC(CCNC(=O)c3ccc(C(=O)NCCC4CCN(C[C@H]5CN[C@H](C)CN5CC(=O)N5CC(C)(C)c6[nH]c(=O)c(Cc7ccc(F)cc7)cc65)CC4)cc3)CC2)CN1]

LCMS (ES, m/z): 618.6 [M/2+H]⁺

¹H NMR (400 MHz, METHANOL-d₄) δ=8.44 (br s, 2H), 8.18 (s, 2H), 7.85 (s, 4H), 7.24 (dd, J=5.6, 8.3 Hz, 4H), 6.99 (t, J=8.7 Hz, 4H), 3.93-3.72 (m, 10H), 3.72-3.62 (m, 2H), 3.53 (br s, 2H), 3.37-3.32 (m, 4H), 3.21 (br d, J=11.8 Hz, 5H), 3.15-2.97 (m, 6H), 2.89 (br d, J=9.6 Hz, 2H), 2.83-2.63 (m, 4H), 2.30 (br d, J=12.0 Hz, 2H), 2.18 (br s, 2H), 2.07-1.92 (m, 2H), 1.79-1.63 (m, 4H), 1.43 (s, 6H), 1.41-1.30 (m, 12H), 1.26 (br d, J=6.3 Hz, 6H), 1.11 (br s, 3H).

### Example 104, N¹,N⁴-bis(2-(1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)ethyl)-N¹,N⁴-dimethylterephthalamide

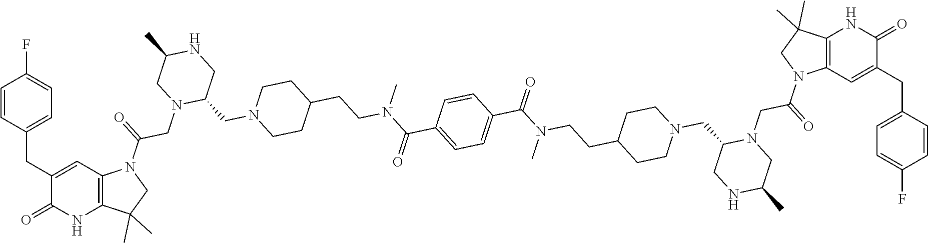 [SMILES: C[C@@H]1CN(CC(=O)N2CC(C)(C)c3[nH]c(=O)c(Cc4ccc(F)cc4)cc32)[C@@H](CN2CCC(CCN(C)C(=O)c3ccc(C(=O)N(C)CCC4CCN(C[C@H]5CN[C@H](C)CN5CC(=O)N5CC(C)(C)c6[nH]c(=O)c(Cc7ccc(F)cc7)cc65)CC4)cc3)CC2)CN1]

LCMS (ES, m/z): 632.6 [M/2+H]P

¹H NMR (400 MHz, METHANOL-d₄) δ=8.40 (br s, 3H), 8.27-8.17 (m, 2H), 7.51-7.44 (m, 2H), 7.42-7.37 (m, 1H), 7.34-7.23 (m, 4H), 7.00 (br t, J=8.6 Hz, 4H), 3.98-3.71 (m, 10H), 3.71-3.56 (m, 4H), 3.56-3.33 (m, 3H), 3.29-3.06 (m, 9H), 3.06-2.83 (m, 8H), 2.83-2.54 (m, 8H), 2.36-2.09 (m, 3H), 2.06-1.54 (m, 6H), 1.46 (s, 4H), 1.38 (br s, 12H), 1.27 (br d, J=5.0 Hz, 8H), 1.18-0.63 (m, 3H).

### Example 105, N,N′-(1,4-phenylenebis(methylene))bis(2-(1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)acetamide)

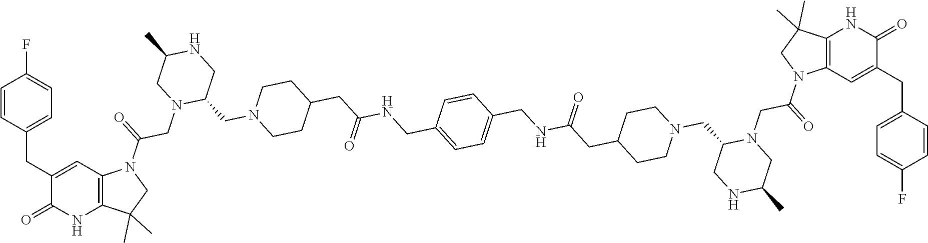 [SMILES: C[C@@H]1CN(CC(=O)N2CC(C)(C)c3[nH]c(=O)c(Cc4ccc(F)cc4)cc32)[C@@H](CN2CCC(CC(=O)NCc3ccc(CNC(=O)CC4CCN(C[C@H]5CN[C@H](C)CN5CC(=O)N5CC(C)(C)c6[nH]c(=O)c(Cc7ccc(F)cc7)cc65)CC4)cc3)CC2)CN1]

LCMS (ES, m/z): 618.6 [M/2+H]⁺

¹H NMR (400 MHz, METHANOL-d₄) δ=8.42 (s, 3H), 8.16 (s, 2H), 7.25-7.19 (m, 7H), 6.98 (t, J=8.8 Hz, 4H), 4.29 (s, 4H), 3.92-3.83 (m, 3H), 3.82-3.74 (m, 6H), 3.71 (s, 4H), 3.47 (br s, 2H), 3.29-3.18 (m, 3H), 3.11-2.97 (m, 6H), 2.93-2.63 (m, 6H), 2.29 (br d, J=12.1 Hz, 2H), 2.16 (br t, J=10.6 Hz, 2H), 2.08-1.91 (m, 6H), 1.75 (br s, 2H), 1.64 (br s, 4H), 1.40 (d, J=8.3 Hz, 12H), 1.26 (d, J=6.4 Hz, 6H), 1.15 (br s, 4H).

### Example 106, N,N′-(1,4-phenylenebis(methylene))bis(2-(1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)-N-methylacetamide)

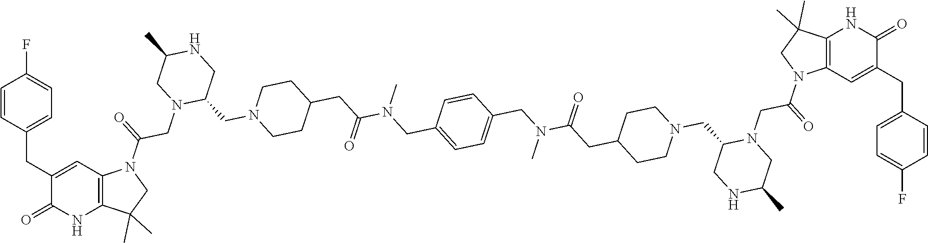 [SMILES: C[C@@H]1CN(CC(=O)N2CC(C)(C)c3[nH]c(=O)c(Cc4ccc(F)cc4)cc32)[C@@H](CN2CCC(CC(=O)N(C)Cc3ccc(CN(C)C(=O)CC4CCN(C[C@H]5CN[C@H](C)CN5CC(=O)N5CC(C)(C)c6[nH]c(=O)c(Cc7ccc(F)cc7)cc65)CC4)cc3)CC2)CN1]

LCMS (ES, m/z): 632.7 [M/2+H]⁺

¹H NMR (400 MHz, METHANOL-d₄) δ=8.43-8.36 (m, 3H), 8.21 (d, J=2.9 Hz, 1H), 8.18 (s, 1H), 7.31 (dd, J=5.6, 8.4 Hz, 1H), 7.27-7.18 (m, 3H), 7.18-7.13 (m, 2H), 7.07-6.91 (m, 5H), 4.64-4.27 (m, 3H), 4.15-3.87 (m, 3H), 3.86-3.67 (m, 9H), 3.67-3.32 (m, 5H), 3.27-3.11 (m, 5H), 3.10-2.92 (m, 4H), 2.91-2.76 (m, 5H), 2.76-2.49 (m, 7H), 2.27-1.80 (m, 9H), 1.79-1.48 (m, 7H), 1.48-1.42 (m, 4H), 1.42-1.36 (m, 5H), 1.35 (d, J=3.5 Hz, 2H), 1.31-1.17 (m, 6H), 1.14-0.74 (m, 4H).

### Example 107, 1,1′-(2,2′-((3R,3′R,6R,6′R)-((((1,3-phenylenebis(oxy))bis(methylene))bis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

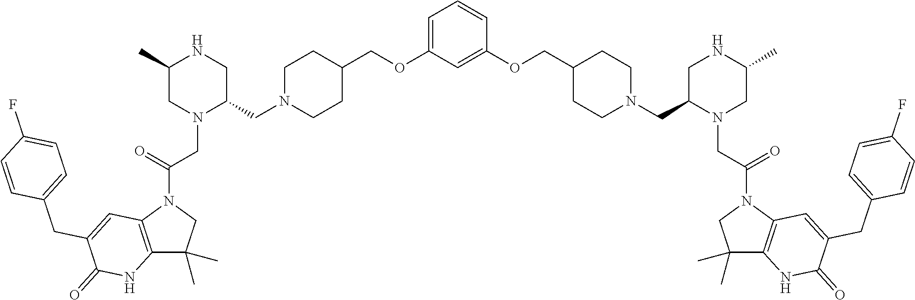 [SMILES: C[C@@H]1CN(CC(=O)N2CC(C)(C)c3[nH]c(=O)c(Cc4ccc(F)cc4)cc32)[C@@H](CN2CCC(COc3cccc(OCC4CCN(C[C@H]5CN[C@H](C)CN5CC(=O)N5CC(C)(C)c6[nH]c(=O)c(Cc7ccc(F)cc7)cc65)CC4)c3)CC2)CN1]

LCMS (ES, m/z): 577.5 [M/2+H]⁺

¹H NMR (400 MHz, METHANOL-d₄) δ=8.46 (br s, 2H), 8.23-8.13 (m, 2H), 7.18 (dd, J=5.4, 8.6 Hz, 4H), 7.13-7.05 (m, 1H), 6.99-6.88 (m, 4H), 6.35-6.16 (m, 3H), 3.87-3.81 (m, 4H), 3.80-3.60 (m, 8H), 3.59-3.37 (m, 6H), 3.26-3.12 (m, 6H), 3.09-2.95 (m, 4H), 2.87 (br d, J=10.6 Hz, 2H), 2.76-2.59 (m, 4H), 2.23-2.03 (m, 4H), 1.90-1.59 (m, 8H), 1.37 (d, J=8.1 Hz, 12H), 1.26 (d, J=6.3 Hz, 6H), 1.21-1.06 (m, 4H).

### Example 108, 1,1′-(2,2′-((3R,3′R,6R,6′R)-((((1,3-phenylenebis(oxy))bis(ethane-2,1-diyl))bis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

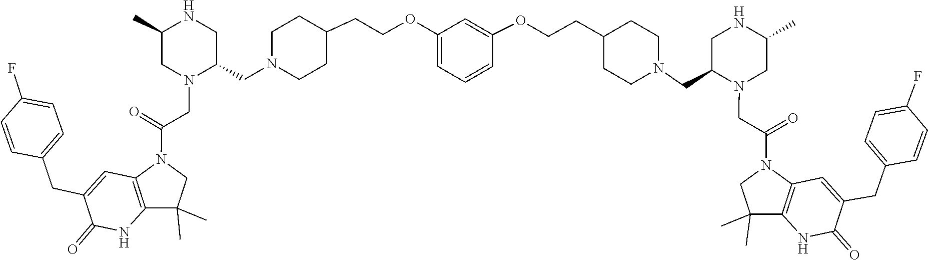 [SMILES: C[C@@H]1CN(CC(=O)N2CC(C)(C)c3[nH]c(=O)c(Cc4ccc(F)cc4)cc32)[C@@H](CN2CCC(CCOc3cccc(OCCC4CCN(C[C@H]5CN[C@H](C)CN5CC(=O)N5CC(C)(C)c6[nH]c(=O)c(Cc7ccc(F)cc7)cc65)CC4)c3)CC2)CN1]

LCMS (ES, m/z): 591.6 [M/2+H]⁺

¹H NMR (400 MHz, METHANOL-d₄) δ=8.47 (br s, 2H), 8.20 (s, 2H), 7.22 (dd, J=5.6, 8.3 Hz, 4H), 7.11 (t, J=8.3 Hz, 1H), 6.95 (t, J=8.7 Hz, 4H), 6.41-6.30 (m, 3H), 3.94-3.62 (m, 16H), 3.53 (br s, 2H), 3.29-2.90 (m, 11H), 2.84 (br s, 2H), 2.79-2.60 (m, 4H), 2.33-1.99 (m, 4H), 1.90 (br s, 2H), 1.71-1.56 (m, 3H), 1.41 (br d, J=5.3 Hz, 18H), 1.26 (br d, J=6.3 Hz, 6H), 1.07 (br s, 4H).

### Example 109, 1,1′-(2,2′-((3R,3′R,6R,6′R)-((((1,3-phenylenebis(oxy))bis(propane-3,1-diyl))bis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

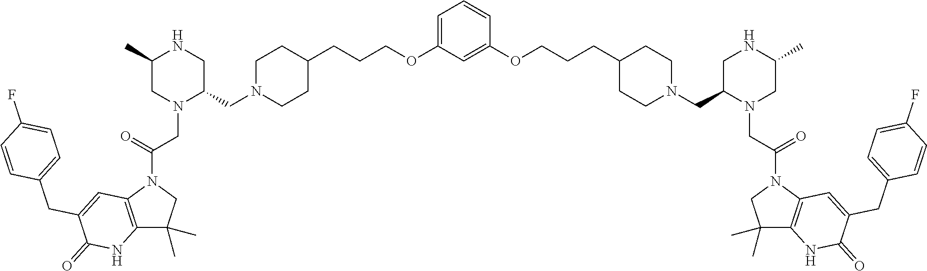 [SMILES: C[C@@H]1CN(CC(=O)N2CC(C)(C)c3[nH]c(=O)c(Cc4ccc(F)cc4)cc32)[C@@H](CN2CCC(CCCOc3cccc(OCCCC4CCN(C[C@H]5CN[C@H](C)CN5CC(=O)N5CC(C)(C)c6[nH]c(=O)c(Cc7ccc(F)cc7)cc65)CC4)c3)CC2)CN1]

LCMS (ES, m/z): 605.6 [M/2+H]⁺

¹H NMR (400 MHz, METHANOL-d₄) δ=8.45 (s, 2H), 8.24 (s, 2H), 7.31-7.18 (m, 4H), 7.10 (t, J=8.1 Hz, 1H), 6.96 (t, J=8.8 Hz, 4H), 6.48-6.39 (m, 3H), 3.93-3.71 (m, 14H), 3.63 (br d, J=17.1 Hz, 4H), 3.30-3.07 (m, 8H), 3.01 (br d, J=11.9 Hz, 2H), 2.89 (br d, J=8.1 Hz, 2H), 2.82-2.62 (m, 4H), 2.37-2.09 (m, 4H), 1.93 (br s, 2H), 1.70-1.42 (m, 13H), 1.41-1.22 (m, 15H), 1.21-0.91 (m, 8H).

### Example 110, 1,1′-(2,2′-((3R,3′R,6R,6′R)-((((1,3-phenylenebis(oxy))bis(1,1-difluoroethane-2,1-diyl))bis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

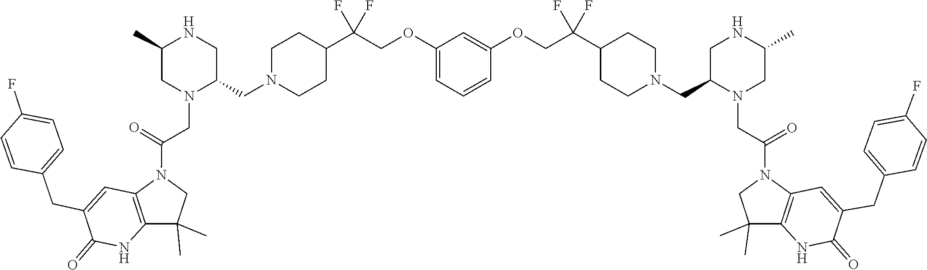 [SMILES: C[C@@H]1CN(CC(=O)N2CC(C)(C)c3[nH]c(=O)c(Cc4ccc(F)cc4)cc32)[C@@H](CN2CCC(C(F)(F)COc3cccc(OCC(F)(F)C4CCN(C[C@H]5CN[C@H](C)CN5CC(=O)N5CC(C)(C)c6[nH]c(=O)c(Cc7ccc(F)cc7)cc65)CC4)c3)CC2)CN1]

LCMS (ES, m/z): 627.5 [M/2+H]⁺

¹H NMR (400 MHz, METHANOL-d4) δ=8.55 (s, 2H), 8.19 (s, 2H), 7.25-7.14 (m, 5H), 6.93 (t, J=8.8 Hz, 4H), 6.57-6.49 (m, 3H), 4.08-3.93 (m, 4H), 3.90-3.56 (m, 13H), 3.44 (br t, J=9.5 Hz, 2H), 3.19-3.01 (m, 8H), 2.95-2.81 (m, 4H), 2.70-2.53 (m, 4H), 2.10-1.87 (m, 6H), 1.73-1.54 (m, 6H), 1.39 (d, J=6.0 Hz, 12H), 1.36-1.16 (m, 11H).

### Example 111, (2R,2′R)-N,N′-(1,4-phenylenebis(methylene))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

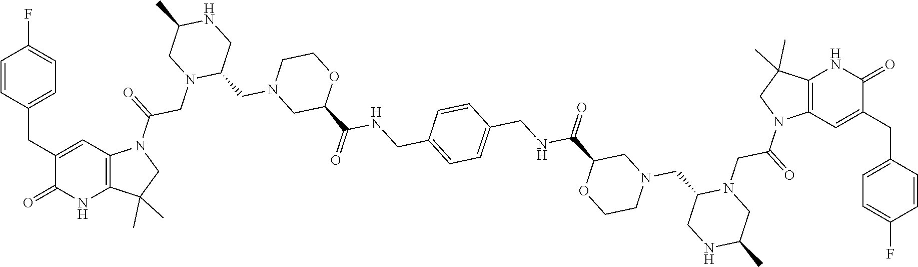 [SMILES: C[C@@H]1CN(CC(=O)N2CC(C)(C)c3[nH]c(=O)c(Cc4ccc(F)cc4)cc32)[C@@H](CN2CCO[C@@H](C(=O)NCc3ccc(CNC(=O)[C@H]4CN(C[C@H]5CN[C@H](C)CN5CC(=O)N5CC(C)(C)c6[nH]c(=O)c(Cc7ccc(F)cc7)cc65)CCO4)cc3)C2)CN1]

LCMS (ES, m/z): 1211.4 [M+H]⁺

¹H NMR (400 MHz, DMSO) d ppm δ 8.33 (s, 2H), 8.17 (t, J=6.2, 2H), 8.11 (s, 2H), 7.28-7.18 (m, 4H), 7.13 (s, 4H), 7.10-7.02 (m, 4H), 4.26 (dd, J=14.8, 6.4, 2H), 4.16 (dd, J=14.8, 6.1, 2H), 3.89-3.50 (m, 16H), 3.39-3.22 (m, 4H), 3.07 (dd, J=28.1, 10.6, 4H), 2.99-2.88 (m, 2H), 2.87-2.73 (m, 4H), 2.65 (d, J=11.5, 2H), 2.58-2.52 (m, 2H), 2.50-2.42 (m, 4H), 2.15-1.95 (m, 4H), 1.78 (t, J=10.6, 2H), 1.28 (d, J=11.0, 12H), 1.07 (d, J=6.1, 6H).

### Example 112, 1,1′-(2,2′-((3R,3′R,6R,6′R)-(((2R,2′R)-((1,3-phenylenebis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

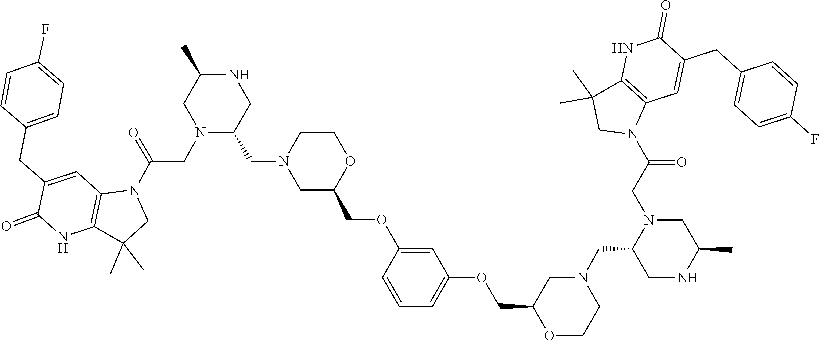 [SMILES: C[C@@H]1CN(CC(=O)N2CC(C)(C)c3[nH]c(=O)c(Cc4ccc(F)cc4)cc32)[C@@H](CN2CCO[C@@H](COc3cccc(OC[C@H]4CN(C[C@H]5CN[C@H](C)CN5CC(=O)N5CC(C)(C)c6[nH]c(=O)c(Cc7ccc(F)cc7)cc65)CCO4)c3)C2)CN1]

LCMS (ES, m/z): 578.7 [M/2+H]⁺

¹H NMR (400 MHz, CD₃OD) δ ppm 8.55 (s, 2H), 8.17 (s, 2H), 7.20 (t, J=8.2 Hz, 1H), 7.13-7.08 (m, 4H), 6.91 (t, J=8.7 Hz, 4H), 6.52 (dd, J=8.3, 1.9 Hz, 2H), 6.44 (s, 1H), 3.96-3.72 (m, 15H), 3.69 (s, 1H), 3.68-3.52 (m, 8H), 3.47 (t, J=11.3 Hz, 2H), 3.29-3.20 (m, 4H), 3.19-3.05 (m, 4H), 2.99 (d, J=10.8 Hz, 2H), 2.79-2.64 (m, 6H), 2.24-2.14 (m, 4H), 1.84 (t, J=10.5 Hz, 2H), 1.48 (d, J=7.3 Hz, 1H), 1.43 (d, J=15.5 Hz, 12H), 1.38 (s, 1H), 1.27 (d, J=5.9 Hz, 6H).

### Example 113, 1,1′-(2,2′-((3R,3′R,6R,6′R)-(((2R,2′R)-((butane-1,4-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

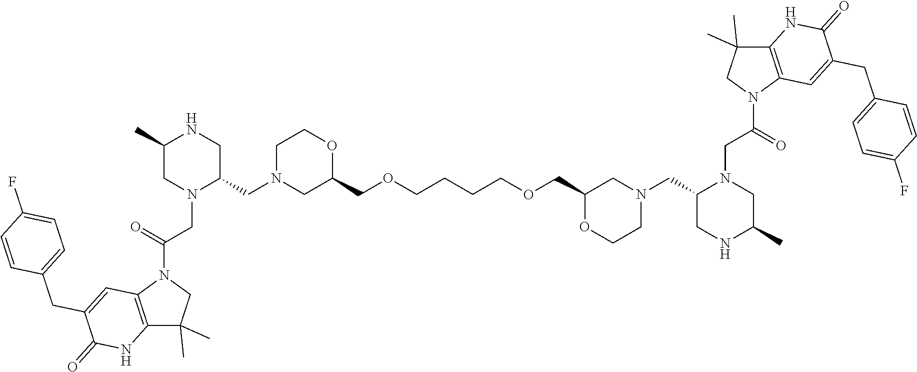 [SMILES: C[C@@H]1CN(CC(=O)N2CC(C)(C)c3[nH]c(=O)c(Cc4ccc(F)cc4)cc32)[C@@H](CN2CCO[C@@H](COCCCCOC[C@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 1136.5 [M+H]⁺

¹H NMR (400 MHz, DMSO) d ppm δ 9.44-9.29 (m, 2H), 8.96-8.81 (m, 4H), 7.92 (d, J=8.8, 2H), 7.28-7.16 (m, 8H), 5.51 (t, J=7.9, 2H), 5.34 (dd, J=8.6, 5.4, 2H), 4.80-4.70 (m, 2H), 4.25 (s, 2H), 4.11 (dd, J=9.3, 4.0, 2H), 3.89 (dd, J=11.5, 6.7, 2H), 3.45-3.39 (m, 4H), 3.25-3.15 (m, 2H), 2.98 (d, J=3.7, 4H), 2.95-2.87 (m, 2H), 2.48 (t, J=5.0, 6H), 2.25-2.11 (m, 4H), 1.89-1.77 (m, 4H), 1.48-1.36 (m, 10H), 1.25-1.20 (m, 32H), 1.07 (d, J=10.8, 12H).

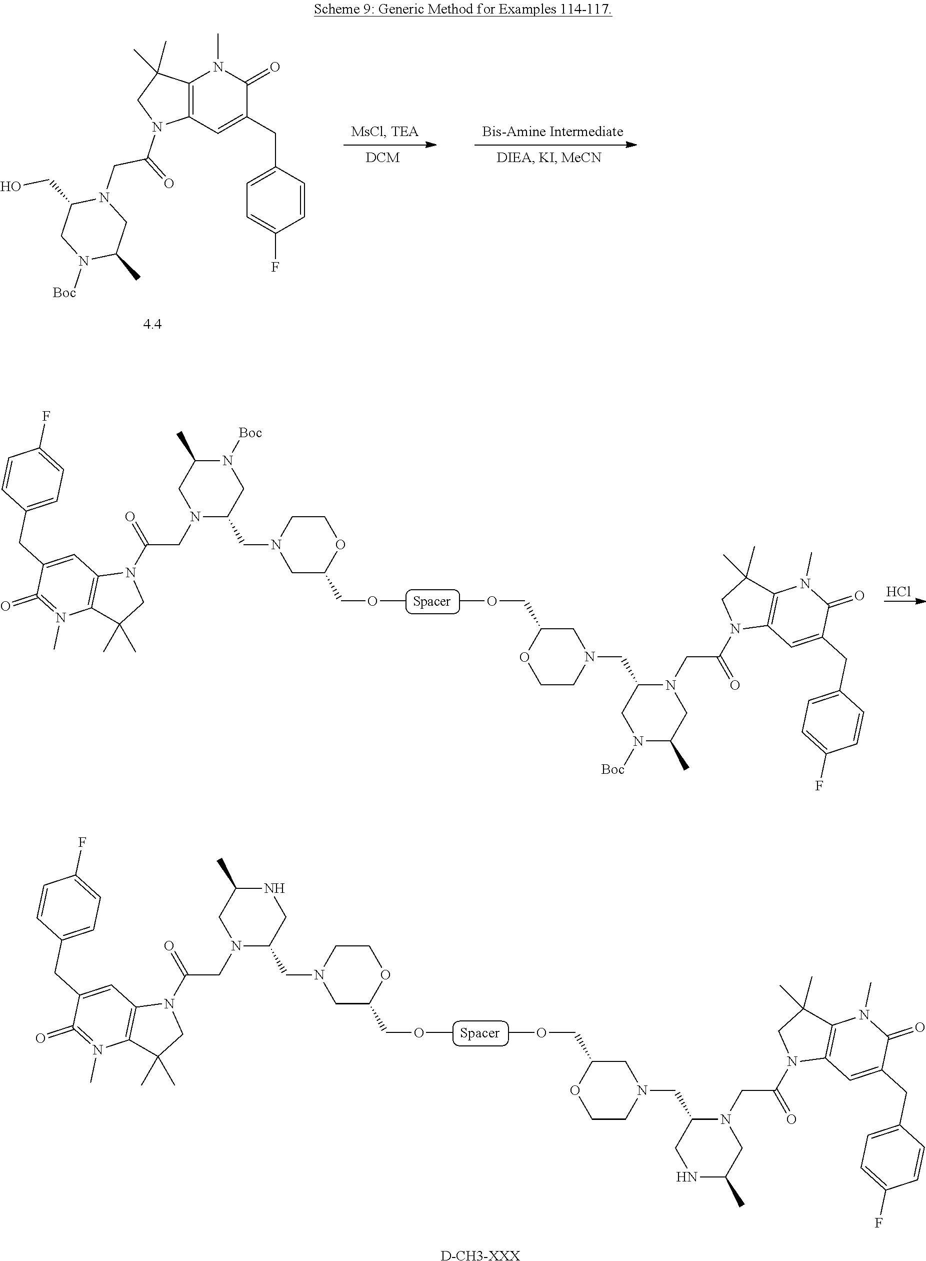

### Example 114, 1,1′-(2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(acetyl))bis(6-(4-fluorobenzyl)-3,3,4-trimethyl-1,2,3,4-tetrahydro-5H-pyrrolo[3,2-b]pyridin-5-one)

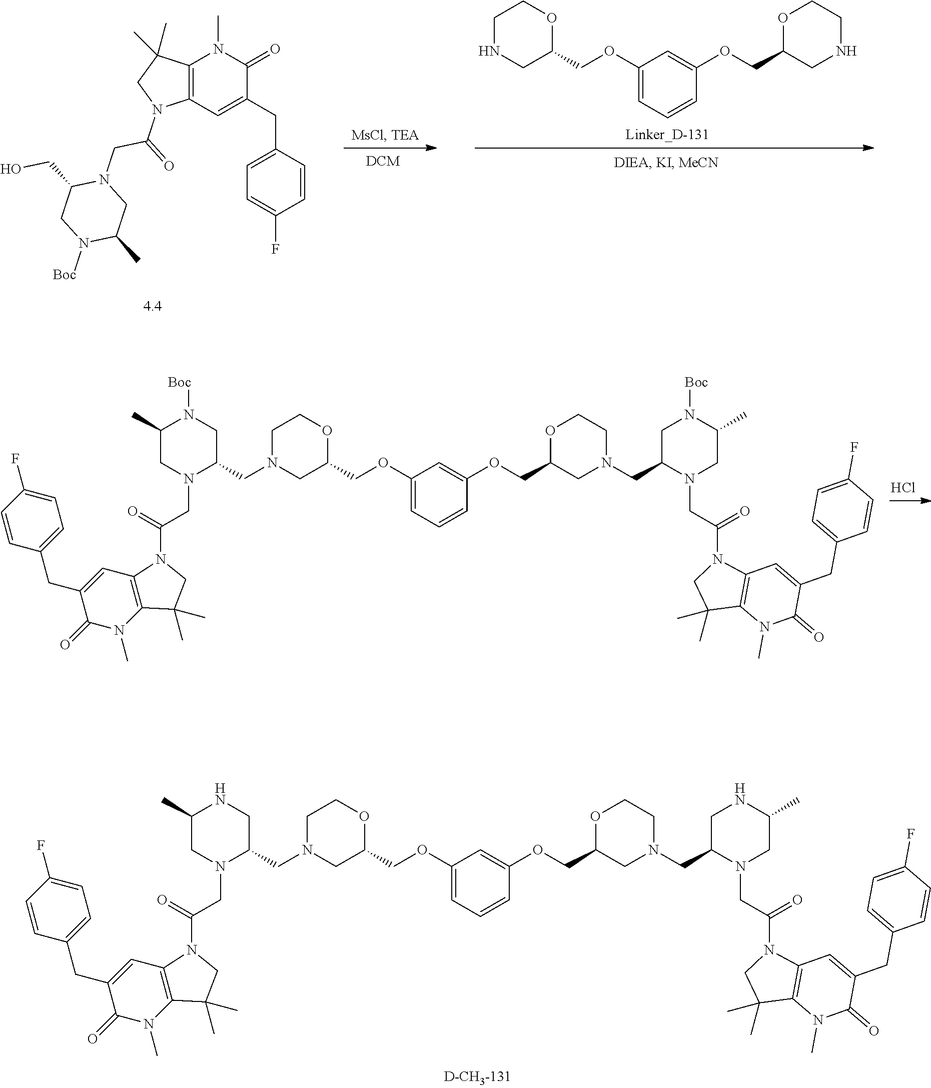

### Step 1 of 2: Synthesis of di-tert-butyl 5,5′-(((2S,2'S)-((1,3-phenylenebis(oxy))bis(methylene))bis(morpholine-2,4-diyl))bis(methylene))(2R,2′R,5S,5'S)-bis(4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-2-methylpiperazine-1-carboxylate)

To a solution of tert-butyl (2R,5R)-4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-(hydroxymethyl)-2-methylpiperazine-1-carboxylate (500 mg, 898 umol, 1 eq) and TEA (136 mg, 1.35 mmol, 187 μL, 1.5 eq) in DCM (5 mL) was added MsCl (123 mg, 1.08 mmol, 83.4 uL, 1.2 eq) at 0° C. The mixture was stirred at 25° C. for 0.5 h after which time TLC indicated complete conversion to a new product (TLC (Petroleum ether/Ethyl acetate=0:1, R_{f}=0.13). The reaction was quenched with saturated NaHCO₃ (20 mL) and then extracted with DCM (10 mL*2). The combined organic phases were washed with brine (10 mL), dried over anhydrous Na₂SO₄, filtered and concentrated in vacuo to give the intermediate product (500 mg, crude) as a yellow solid.

A mixture of the intermediate product (506 mg, 881 umol, 2.1 eq), (2S)-2-[[3-[[(2S)-morpholin-2-yl]methoxy]phenoxy]methyl]morpholine (Linker D-131, 160 mg, 419 umol, 1 eq, 2 HCl), DIEA (271 mg, 2.10 mmol, 365 μL, 5 eq) and KI (139 mg, 839 umol, 2 eq) in MeCN (5 mL) was stirred at 80° C. for 12 h after which time LCMS indicated formation of a product of target mass. The mixture was filtered and the filtrate was concentrated to dryness. The residue was purified by preparative TLC (Dichloromethane/Methanol=20:1) to give the title compound (470 mg, crude) as a yellow solid.

LCMS (ES, m/z): 1385.8 [M+H]⁺

### Step 2 of 2: Synthesis of 1,1′-(2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(acetyl))bis(6-(4-fluorobenzyl)-3,3,4-trimethyl-1,2,3,4-tetrahydro-5H-pyrrolo[3,2-b]pyridin-5-one)

To a mixture of di-tert-butyl 5,5′-(((2S,2'S)-((1,3-phenylenebis(oxy))bis(methylene))bis(morpholine-2,4-diyl))bis(methylene))(2R,2′R,5S,5'S)-bis(4-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-2-methylpiperazine-1-carboxylate) (460 mg, 332 umol, 1 eq) in EtOAc (8 mL) was added HCl/EtOAc (4M, 8 mL, 96 eq) dropwise at 15° C. The mixture was stirred at 15° C. for 1 h after which time LCMS indicated complete conversion to a product of target mass. The mixture was filtered and the filtrate was concentrated to dryness. The residue was purified by preparative HPLC (column: Phenomenex Luna C18 200 mm*40 mm*10 um; mobile phase: [water(0.2% FA)-ACN]; B %: 1%-50%, 8 min) to give the title compound (24.2 mg, 18.5 umol, 13% yield, 97% purity, 2 FA) as a yellow solid.

LCMS (ES, m/z): 1185.7 [M+H]⁺

¹H NMR (400 MHz, CD₃OD, 298 K) δ (ppm)=8.53 (s, 2H), 8.24 (s, 2H), 7.24-7.17 (m, 4H), 7.10 (t, J=8.2 Hz, 1H), 7.01-6.93 (m, 4H), 6.43 (dd, J=2.3, 8.3 Hz, 2H), 6.35 (t, J=2.1 Hz, 1H), 3.87-3.82 (m, 4H), 3.80-3.72 (m, 13H), 3.64-3.58 (m, 7H), 3.49-3.35 (m, 4H), 3.29-3.21 (m, 4H), 3.09-2.93 (m, 4H), 2.84-2.61 (m, 8H), 2.17 (br dd, J=2.7, 13.2 Hz, 2H), 2.04-1.94 (m, 4H), 1.48 (d, J=4.3 Hz, 12H), 1.26 (d, J=6.5 Hz, 6H).

Examples 115-117 were prepared according to the same procedure as Example 114, replacing 1,3-bis(((S)-morpholin-2-yl)methoxy)benzene (Linker D-131) with the appropriate bis-amine linker. The compounds were found to have characterizing data as set forth below.

### Example 115, 1,1′-(2,2′-((3R,3′R,6R,6′R)-(((2S,2'S)-(((methylenebis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3,4-trimethyl-1,2,3,4-tetrahydro-5H-pyrrolo[3,2-b]pyridin-5-one)

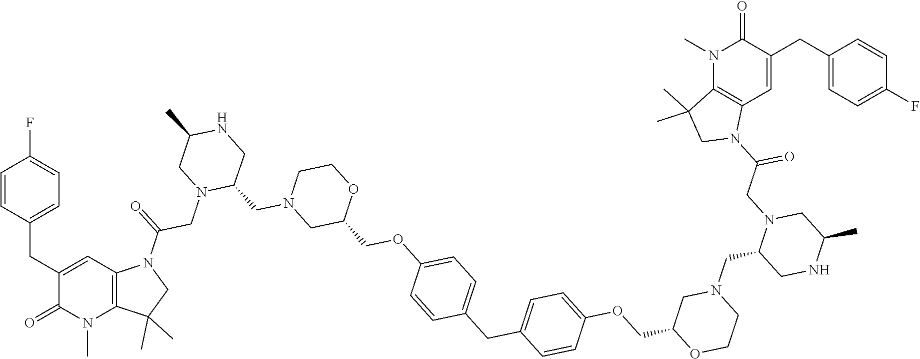 [SMILES: C[C@@H]1CN(CC(=O)N2CC(C)(C)c3c2cc(Cc2ccc(F)cc2)c(=O)n3C)[C@@H](CN2CCO[C@H](COc3ccc(Cc4ccc(OC[C@@H]5CN(C[C@H]6CN[C@H](C)CN6CC(=O)N6CC(C)(C)c7c6cc(Cc6ccc(F)cc6)c(=O)n7C)CCO5)cc4)cc3)C2)CN1]

LCMS (ES, m/z): 638.5 [M/2+H]⁺

¹H NMR (400 MHz, METHANOL-d₄) δ ppm 8.06 (s, 2H) 7.10-7.18 (m, 4H) 7.07 (br d, J=8.33 Hz, 4H) 6.94-7.02 (m, 4H) 6.79 (br d, J=8.33 Hz, 4H) 3.87-4.24 (m, 12H) 3.72-3.86 (m, 12H) 3.63-3.71 (m, 8H) 3.43-3.62 (m, 6H) 3.33-3.41 (m, 2H) 2.97-3.28 (m, 14H) 1.53 (br d, J=8.77 Hz, 12H) 1.30 (br d, J=6.14 Hz, 6H).

### Example 116, 1,1′-(2,2′-((3R,3′R,6R,6′R)-(((2S,2'S)-((hexa-2,4-diyne-1,6-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3,4-trimethyl-1,2,3,4-tetrahydro-5H-pyrrolo[3,2-b]pyridin-5-one)

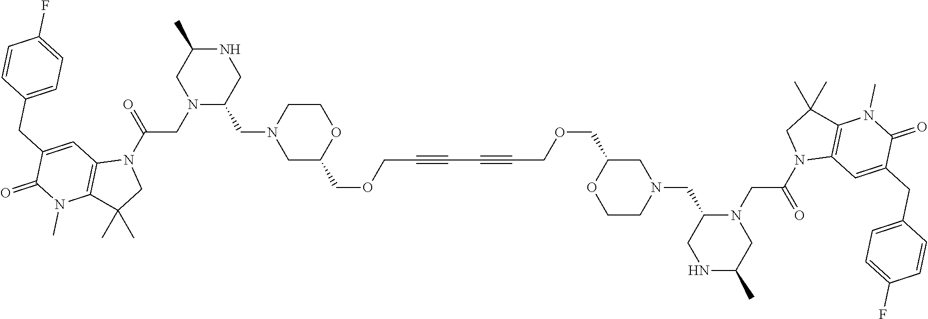 [SMILES: C[C@@H]1CN(CC(=O)N2CC(C)(C)c3c2cc(Cc2ccc(F)cc2)c(=O)n3C)[C@@H](CN2CCO[C@H](COCC#CC#CCOC[C@@H]3CN(C[C@H]4CN[C@H](C)CN4CC(=O)N4CC(C)(C)c5c4cc(Cc4ccc(F)cc4)c(=O)n5C)CCO3)C2)CN1]

LCMS (ES, m/z): 593.5 [M/2+H]⁺

¹H NMR (400 MHz, METHANOL-d₄) δ=8.06 (s, 2H), 7.24 (dd, J=5.5, 8.6 Hz, 4H), 7.10-7.03 (m, 4H), 4.27 (s, 4H), 4.14 (br d, J=11.8 Hz, 2H), 4.02-3.96 (m, 2H), 3.93-3.72 (m, 15H), 3.71-3.62 (m, 10H), 3.61-3.48 (m, 6H), 3.38-3.32 (m, 2H), 3.28-3.08 (m, 9H), 3.02 (br dd, J=10.3, 15.1 Hz, 6H), 1.53 (d, J=8.8 Hz, 12H), 1.30 (d, J=6.6 Hz, 5H), 1.32-1.28 (m, 1H).

### Example 117, 1,1′-(2,2′-((3R,3′R,6R,6′R)-(((2S,2'S)-(((1,4-phenylenebis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3,4-trimethyl-1,2,3,4-tetrahydro-5H-pyrrolo[3,2-b]pyridin-5-one)

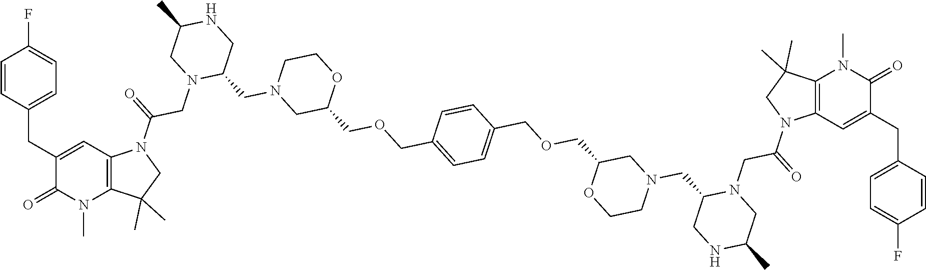 [SMILES: C[C@@H]1CN(CC(=O)N2CC(C)(C)c3c2cc(Cc2ccc(F)cc2)c(=O)n3C)[C@@H](CN2CCO[C@H](COCc3ccc(COC[C@@H]4CN(C[C@H]5CN[C@H](C)CN5CC(=O)N5CC(C)(C)c6c5cc(Cc5ccc(F)cc5)c(=O)n6C)CCO4)cc3)C2)CN1]

LCMS (ES, m/z): 607.5 [M/2+H]⁺

¹H NMR (400 MHz, METHANOL-d₄) δ ppm 8.04 (s, 2H) 7.22 (s, 4H) 7.16 (dd, J=8.55, 5.48 Hz, 4H) 6.97-7.04 (m, 4H) 4.50 (s, 4H) 4.09-4.20 (m, 2H) 3.97 (br d, J=10.52 Hz, 2H) 3.85-3.92 (m, 2H) 3.68-3.83 (m, 14H) 3.65 (s, 6H) 3.59-3.64 (m, 2H) 3.46-3.58 (m, 6H) 3.32-3.39 (m, 2H) 2.90-3.28 (m, 16H) 1.51 (d, J=15.35 Hz, 12H) 1.30 (d, J=6.14 Hz, 6H).

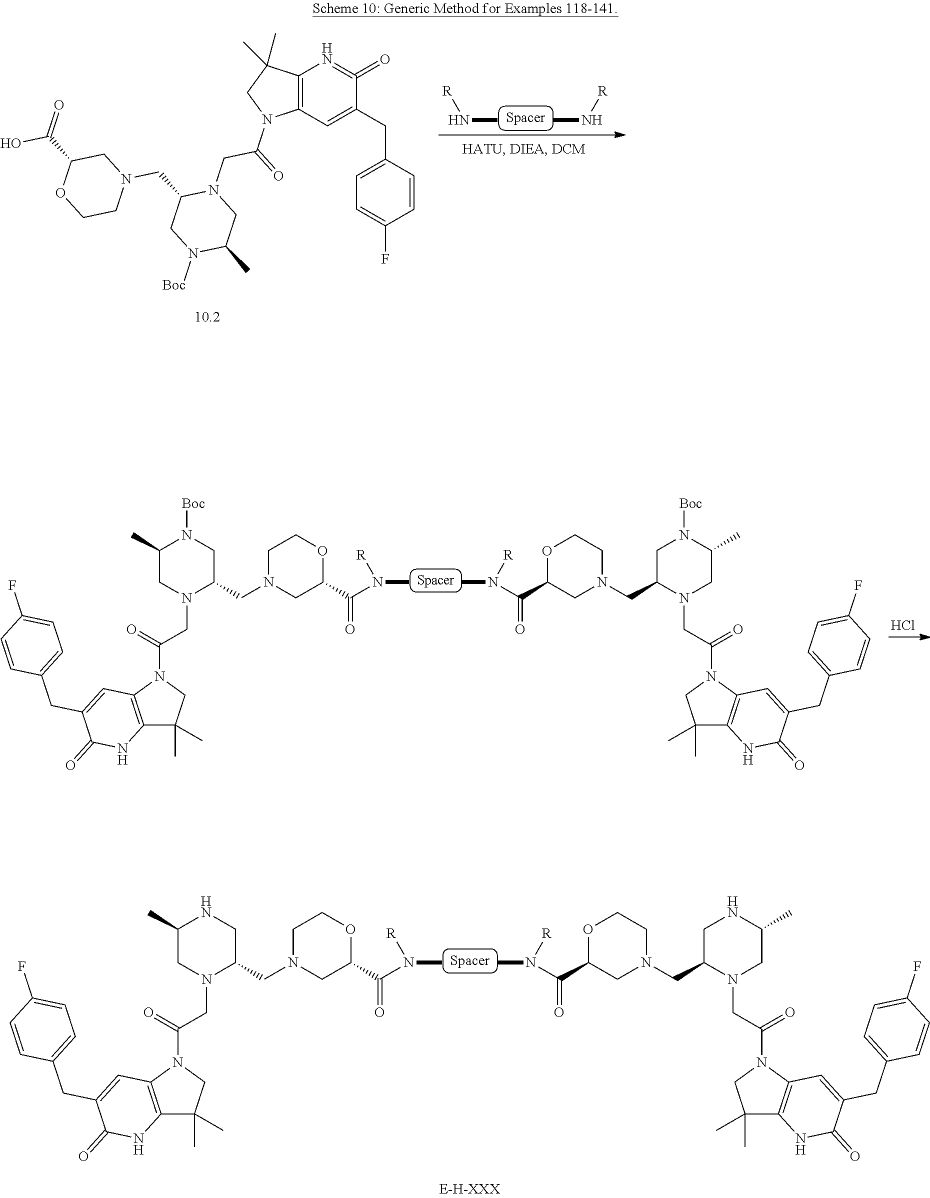

### Example 118, 1,1′-(2,2′-((3R,3′R,6R,6′R)-(((2S,2'S)-(piperazine-1,4-dicarbonyl)bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

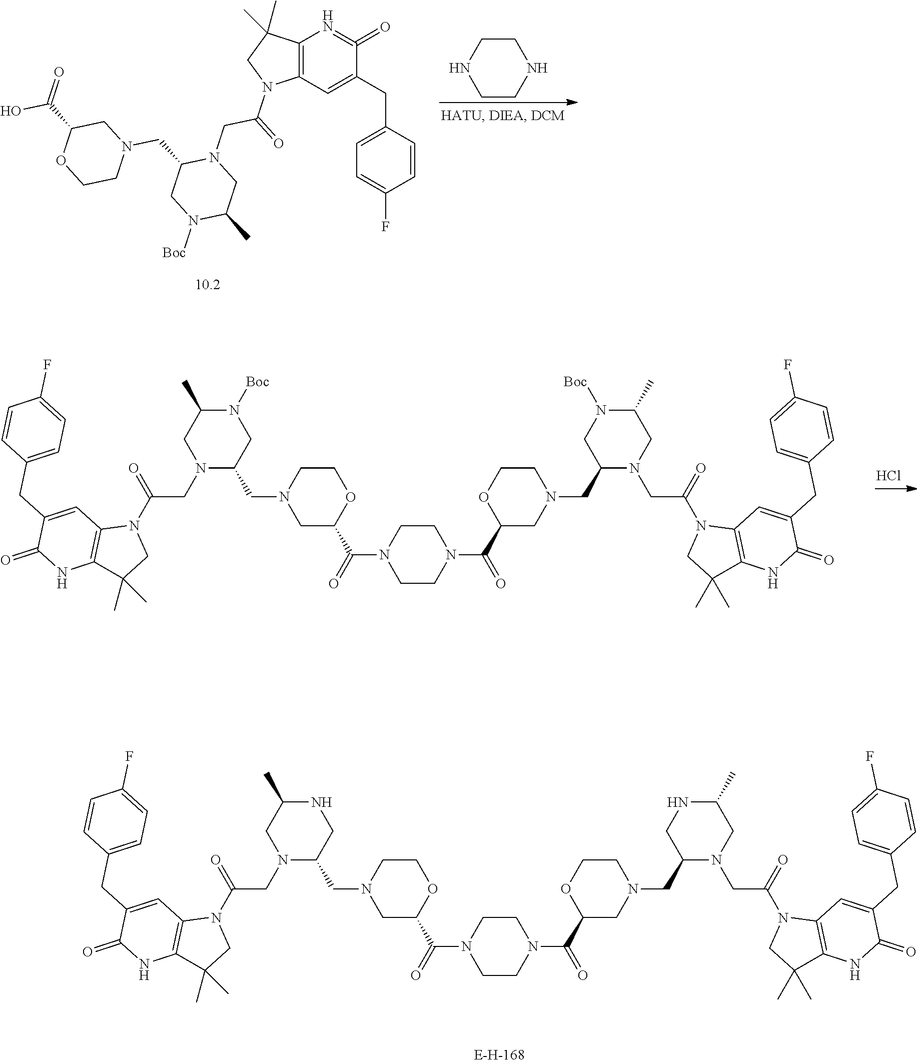

### Step 1 of 2: Synthesis of di-tert-butyl 5,5′-(((2S,2'S)-(piperazine-1,4-dicarbonyl)bis(morpholine-2,4-diyl))bis(methylene))(2R,2′R,5S,5'S)-bis(4-(2-(6-(4-fluorobenzyl)-3,3-dimethyl-5-oxo-2,3,4,5-tetrahydro-1H-pyrrolo[3,2-b]pyridin-1-yl)-2-oxoethyl)-2-methylpiperazine-1-carboxylate)

To a solution of (S)-4-(((2S,5R)-4-(tert-butoxycarbonyl)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxylic acid (150 mg, 229 umol, 2.6 eq) in DCM (3 mL) were added piperazine (7.58 mg, 88.0 umol, 1 eq), DIEA (45.5 mg, 352 umol, 61.3 uL, 4 eq), and HATU (83.6 mg, 220 umol, 2.5 eq). The mixture was stirred at 15° C. for 12 h after which time LCMS indicated complete conversion to a product of target mass. The mixture was concentrated under reduced pressure and the residue was purified by column chromatography (SiO₂, Dichloromethane/Methanol=1:0 to 0:1) to give the title compound (190 mg, crude) as a yellow oil.

LCMS (ES, m/z): 1362.1 [M+H]⁺

### Step 2 of 2: Synthesis of 1,1′-(2,2′-((3R,3′R,6R,6′R)-(((2S,2'S)-(piperazine-1,4-dicarbonyl)bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

To a solution of di-tert-butyl 5,5′-(((2S,2'S)-(piperazine-1,4-dicarbonyl)bis(morpholine-2,4-diyl))bis(methylene))(2R,2′R,5S,5'S)-bis(4-(2-(6-(4-fluorobenzyl)-3,3-dimethyl-5-oxo-2,3,4,5-tetrahydro-1H-pyrrolo[3,2-b]pyridin-1-yl)-2-oxoethyl)-2-methylpiperazine-1-carboxylate) (190 mg, 139 umol, 1 eq) in EtOAc (2 mL) was added HCl/EtOAc (4 M, 3 mL). The mixture was stirred at 15° C. for 1 h after which time LCMS indicated conversion to a product of target mass. The reaction mixture was filtered and the filtrate concentrated under high vacuum. The residue was purified by preparative HPLC (TFA condition; column: Phenomenex Gemini-NX 150*30 mm*5 um; mobile phase: [water(0.1% TFA)-ACN]; B %: 10%-40%, 9 min) to give the title compound (29.2 mg, 21.0 umol, 15% yield, 100% purity, 2 TFA) as an off-white solid.

LCMS (ES, m/z): 581.5 [M/2+H]⁺

¹H NMR (400 MHz, METHANOL-d₄) δ=8.14 (br s, 2H), 7.33-7.18 (m, 4H), 7.07-6.94 (m, 4H), 4.05-3.84 (m, 7H), 3.84-3.52 (m, 13H), 3.51-3.35 (m, 7H), 3.25 (br d, J=13.4 Hz, 5H), 3.18-2.94 (m, 10H), 2.89 (br s, 4H), 2.44 (br s, 4H), 1.51 (br s, 4H), 1.45-1.36 (m, 8H), 1.29 (br d, J=6.3 Hz, 6H).

Examples 119-141 were prepared according to the same procedure as E-H-168, replacing piperazine with the appropriate bis-amine. The compounds were found to have characterizing data as set forth below.

### Example 119, (2S,2'S)-N,N′-(octane-1,8-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

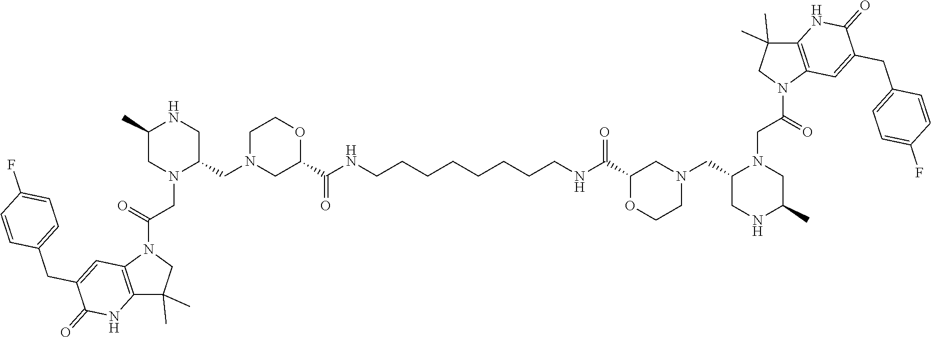 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCCCCCCCNC(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 610.4 [M/2+H]

¹H NMR (400 MHz, METHANOL-d₄) δ=8.55 (br s, 1H), 8.20 (s, 2H), 7.25 (dd, J=5.5, 8.5 Hz, 4H), 6.99 (t, J=8.8 Hz, 4H), 4.13-3.59 (m, 18H), 3.43-3.32 (m, 3H), 3.24-2.97 (m, 11H), 2.93-2.77 (m, 6H), 2.67-2.55 (m, 4H), 2.15 (br d, J=11.1 Hz, 2H), 2.03-1.88 (m, 4H), 1.46-1.35 (m, 16H), 1.26 (br s, 8H), 1.16 (br d, J=6.3 Hz, 6H).

### Example 120, (2S,2'S)-N,N′-(decane-1,10-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

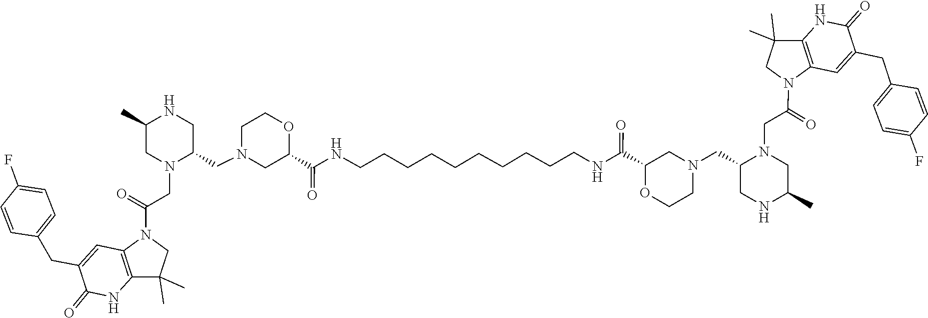 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCCCCCCCCCNC(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 624.4 [M/2+H]⁺

¹H NMR (400 MHz, METHANOL-d₄) δ=8.04-7.85 (m, 2H), 7.25-7.08 (m, 4H), 7.01-6.83 (m, 4H), 4.05-3.93 (m, 2H), 3.93-3.80 (m, 4H), 3.80-3.67 (m, 7H), 3.67-3.45 (m, 6H), 3.44-3.23 (m, 4H), 3.19-3.09 (m, 6H), 3.09-3.03 (m, 4H), 3.02-2.77 (m, 8H), 2.77-2.64 (m, 2H), 2.63-2.41 (m, 3H), 1.39 (br d, J=6.6 Hz, 4H), 1.36-1.25 (m, 12H), 1.19 (br d, J=6.5 Hz, 18H).

### Example 121, (2S,2'S)-N,N′-(((oxybis(ethane-2,1-diyl))bis(oxy))bis(ethane-2,1-diyl))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

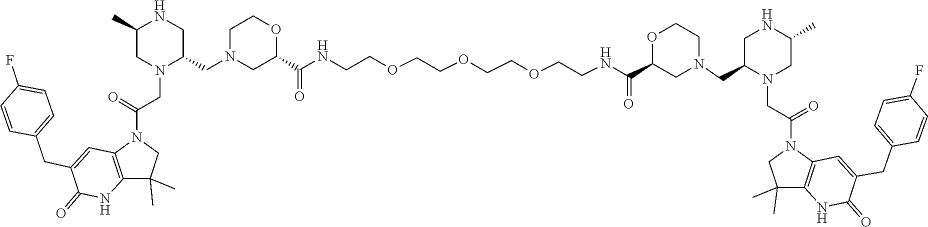 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCOCCOCCOCCNC(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 634.4 [M/2+H]⁺

¹H NMR (400 MHz, METHANOL-d₄) δ=8.60-8.45 (m, 2H), 8.24-8.14 (m, 2H), 7.29-7.20 (m, 4H), 7.08-6.95 (m, 4H), 3.90-3.75 (m, 12H), 3.73-3.63 (m, 4H), 3.60-3.45 (m, 14H), 3.44-3.34 (m, 4H), 3.29-3.22 (m, 4H), 3.16-2.94 (m, 6H), 2.84-2.59 (m, 6H), 2.28-2.13 (m, 2H), 2.07-1.90 (m, 4H), 1.42-1.34 (m, 12H), 1.32-1.22 (m, 6H).

### Example 122, (2S,2'S)-N,N′-(1,3-phenylenebis(methylene))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

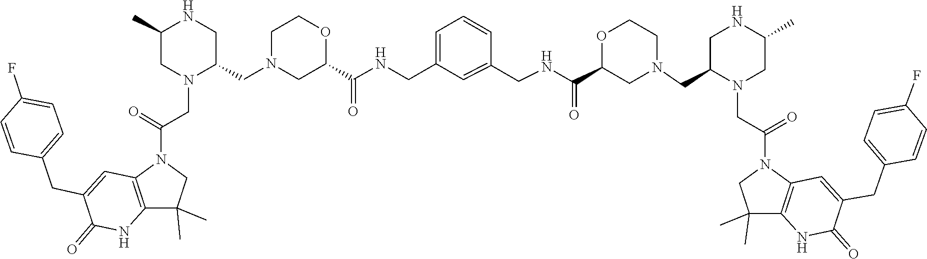 [SMILES: C[C@@H]1CN(CC(=O)N2CC(C)(C)c3[nH]c(=O)c(Cc4ccc(F)cc4)cc32)[C@@H](CN2CCO[C@H](C(=O)NCc3cccc(CNC(=O)[C@@H]4CN(C[C@H]5CN[C@H](C)CN5CC(=O)N5CC(C)(C)c6[nH]c(=O)c(Cc7ccc(F)cc7)cc65)CCO4)c3)C2)CN1]

LCMS (ES, m/z): 606.3 [M/2+H]

¹H NMR (400 MHz, METHANOL-d₄) δ=8.08-7.97 (m, 2H), 7.33-7.08 (m, 8H), 7.07-6.87 (m, 4H), 4.49-4.27 (m, 4H), 4.22-4.08 (m, 2H), 4.07-3.56 (m, 18H), 3.56-3.33 (m, 6H), 3.29-3.22 (m, 2H), 3.21-2.98 (m, 9H), 2.98-2.85 (m, 2H), 2.84-2.61 (m, 3H), 1.49-1.33 (m, 12H), 1.29 (d, J=6.5 Hz, 6H).

### Example 123, (2S,2'S)-N,N′-(piperazine-1,4-diylbis(propane-3,1-diyl))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

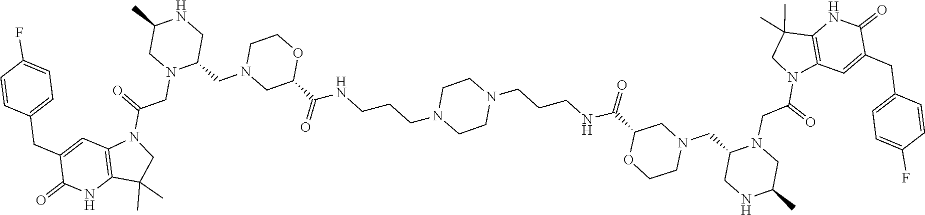 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCCN3CCN(CCCNC(=O)[C@@H]4CN(C[C@H]5CN[C@H](C)CN5CC(=O)N5CC(C)(C)c6[nH]c(=O)c(Cc7ccc(F)cc7)cc65)CCO4)CC3)C2)CN1]

LCMS (ES, m/z): 638.4 [M/2+H]⁺

¹H NMR (400 MHz, METHANOL-d₄) δ=8.20-8.14 (m, 2H), 7.30-7.21 (m, 4H), 7.08-6.96 (m, 4H), 3.95-3.85 (m, 6H), 3.84-3.78 (m, 7H), 3.77-3.59 (m, 6H), 3.55-3.36 (m, 6H), 3.27-3.09 (m, 12H), 3.09-2.89 (m, 10H), 2.66 (s, 11H), 1.85-1.74 (m, 4H), 1.45-1.37 (m, 12H), 1.30-1.25 (m, 6H).

### Example 124, (2S,2'S)-N,N′-(hexane-1,6-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-methylmorpholine-2-carboxamide)

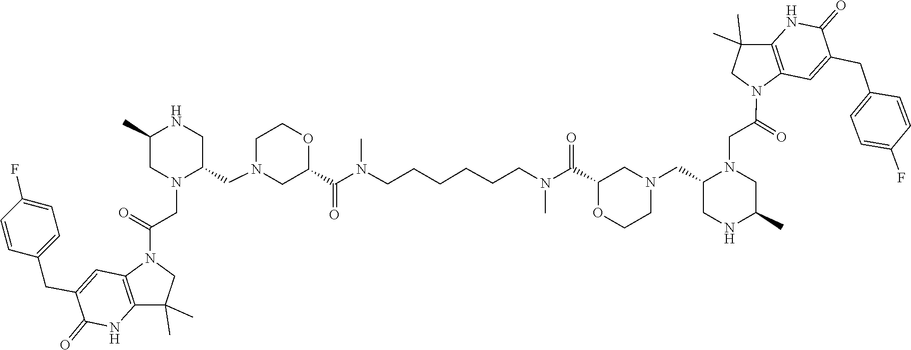 [SMILES: C[C@@H]1CN(CC(=O)N2CC(C)(C)c3[nH]c(=O)c(Cc4ccc(F)cc4)cc32)[C@@H](CN2CCO[C@H](C(=O)N(C)CCCCCCN(C)C(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 610.4 [M/2+H]⁺

¹H NMR (400 MHz, METHANOL-d₄) δ=8.20-8.04 (m, 2H), 7.32-7.15 (m, 4H), 7.10-6.93 (m, 4H), 4.08-3.88 (m, 2H), 3.87-3.49 (m, 14H), 3.50-3.32 (m, 6H), 3.27 (br s, 30H), 1.59-1.36 (m, 16H), 1.33-1.15 (m, 10H).

### Example 125, (2S,2'S)-N,N′-(Propane-1,3-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

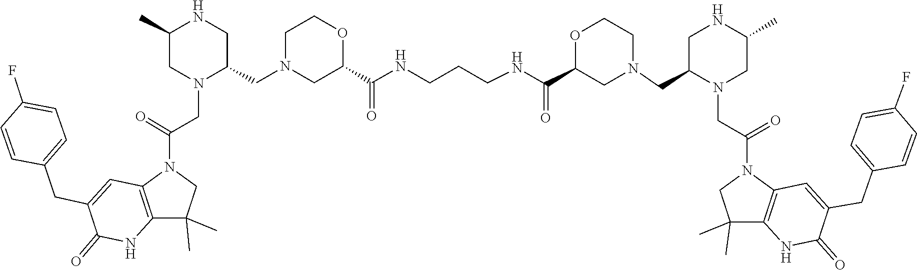 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCCNC(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 575.3 [M/2+H]⁺

¹H NMR (400 MHz, METHANOL-d₄) δ=8.14-8.07 (m, 2H), 8.04-7.99 (m, 1H), 7.26-7.18 (m, 4H), 7.00 (t, J=8.7 Hz, 4H), 3.99-3.91 (m, 2H), 3.91-3.86 (m, 2H), 3.86-3.75 (m, 10H), 3.74-3.67 (m, 2H), 3.67-3.57 (m, 2H), 3.57-3.36 (m, 4H), 3.28-3.13 (m, 10H), 3.12-2.99 (m, 6H), 2.94-2.79 (m, 4H), 2.62-2.45 (m, 2H), 2.39-2.27 (m, 2H), 1.72-1.60 (m, 2H), 1.39 (d, J=9.6 Hz, 11H), 1.28 (d, J=6.5 Hz, 6H).

### Example 126, (2S,2'S)-N,N′-(butane-1,4-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

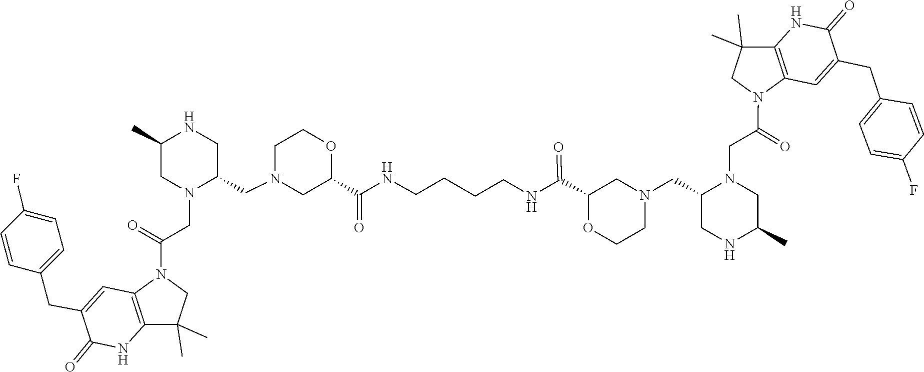 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCCCNC(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 582.3 [M/2+H]⁺

¹H NMR (400 MHz, METHANOL-d₄) δ=8.11-8.00 (m, 2H), 7.29-7.17 (m, 4H), 7.07-6.95 (m, 4H), 4.19-4.05 (m, 2H), 4.04-3.91 (m, 4H), 3.85 (br d, J=10.8 Hz, 2H), 3.79 (br s, 8H), 3.75-3.57 (m, 4H), 3.50-3.33 (m, 6H), 3.28 (br s, 2H), 3.26-3.13 (m, 6H), 3.13-2.84 (m, 8H), 2.81-2.57 (m, 4H), 1.55 (br s, 4H), 1.41 (br d, J=4.9 Hz, 12H), 1.29 (d, J=6.5 Hz, 6H).

### Example 127, (2S,2'S)-N,N′-(butane-1,4-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-methylmorpholine-2-carboxamide)

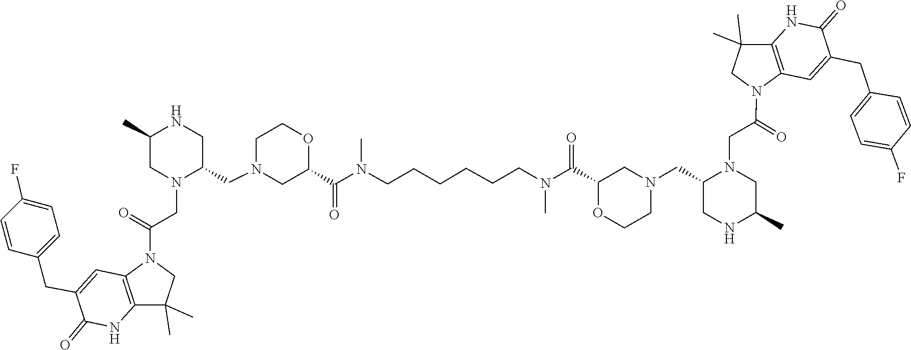 [SMILES: C[C@@H]1CN(CC(=O)N2CC(C)(C)c3[nH]c(=O)c(Cc4ccc(F)cc4)cc32)[C@@H](CN2CCO[C@H](C(=O)N(C)CCCCCCN(C)C(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 596.3 [M/2+H]⁺

¹H NMR (400 MHz, METHANOL-d₄) δ=8.10 (br d, J=9.3 Hz, 2H), 7.27-7.19 (m, 4H), 7.00 (br t, J=8.6 Hz, 4H), 3.95-3.69 (m, 16H), 3.64 (br s, 4H), 3.50-3.35 (m, 4H), 3.28-2.88 (m, 22H), 2.84 (br d, J=5.3 Hz, 4H), 1.57-1.33 (m, 18H), 1.28 (br d, J=6.3 Hz, 6H).

### Example 128, (2S,2'S)-N,N′-(ethane-1,2-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

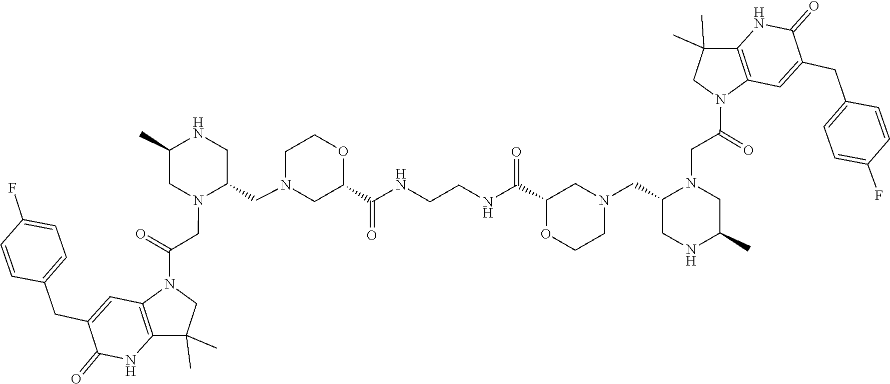 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCNC(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 568.3 [M/2+H]⁺

¹H NMR (400 MHz, METHANOL-d₄) δ=8.17 (br s, 1H), 8.04 (s, 2H), 7.22 (dd, J=5.6, 8.2 Hz, 4H), 7.00 (t, J=8.8 Hz, 4H), 4.08 (br d, J=10.8 Hz, 2H), 3.99-3.82 (m, 7H), 3.78 (br s, 8H), 3.74-3.53 (m, 5H), 3.47-3.36 (m, 6H), 3.27 (br d, J=13.3 Hz, 4H), 3.20-2.95 (m, 9H), 2.84 (br d, J=15.8 Hz, 2H), 2.64 (br s, 3H), 1.40 (d, J=2.9 Hz, 12H), 1.29 (br d, J=6.5 Hz, 6H).

### Example 129, (S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-(1-((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carbonyl)piperidin-4-yl)morpholine-2-carboxamide

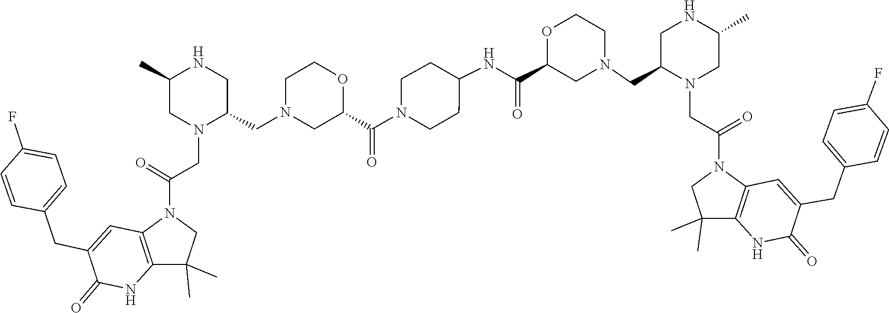 [SMILES: C[C@@H]1CN(CC(=O)N2CC(C)(C)c3[nH]c(=O)c(Cc4ccc(F)cc4)cc32)[C@@H](CN2CCO[C@H](C(=O)NC3CCN(C(=O)[C@@H]4CN(C[C@H]5CN[C@H](C)CN5CC(=O)N5CC(C)(C)c6[nH]c(=O)c(Cc7ccc(F)cc7)cc65)CCO4)CC3)C2)CN1]

LCMS (ES, m/z): 588.3 [M/2+H]⁺

¹H NMR (400 MHz, METHANOL-d₄) δ=8.22-8.10 (m, 2H), 7.30-7.19 (m, 4H), 7.06-6.94 (m, 4H), 4.43-4.17 (m, 1H), 3.90 (br s, 5H), 3.81 (br t, J=14.7 Hz, 12H), 3.71-3.45 (m, 6H), 3.37 (br d, J=14.9 Hz, 3H), 3.27 (br s, 3H), 3.21-2.79 (m, 14H), 2.78-2.22 (m, 3H), 1.91-1.60 (m, 2H), 1.40 (br d, J=15.0 Hz, 14H), 1.29 (br d, J=6.0 Hz, 6H).

### Example 130, (2S,2'S)-N,N′-(Propane-1,3-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-methylmorpholine-2-carboxamide)

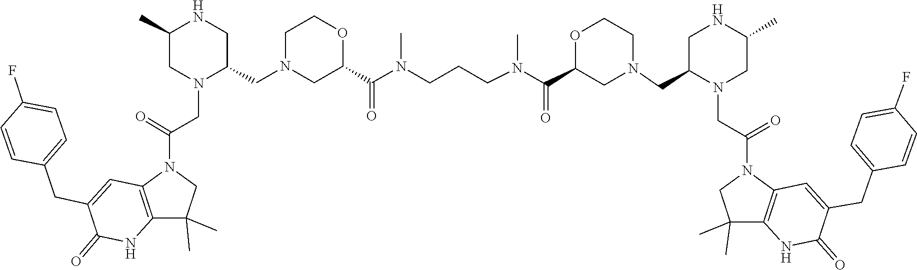 [SMILES: C[C@@H]1CN(CC(=O)N2CC(C)(C)c3[nH]c(=O)c(Cc4ccc(F)cc4)cc32)[C@@H](CN2CCO[C@H](C(=O)N(C)CCCN(C)C(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 589.3 [M/2+H]⁺

¹H NMR (400 MHz, METHANOL-d₄) δ=8.24-7.99 (m, 2H), 7.22 (br dd, J=5.3, 8.6 Hz, 4H), 7.06-6.93 (m, 4H), 3.99-3.89 (m, 3H), 3.89-3.80 (m, 6H), 3.77 (br s, 5H), 3.72 (br d, J=9.6 Hz, 5H), 3.51-3.33 (m, 5H), 3.27 (br s, 3H), 3.22-2.91 (m, 18H), 2.89-2.52 (m, 7H), 1.85-1.63 (m, 2H), 1.45-1.34 (m, 12H), 1.29 (br d, J=6.4 Hz, 6H).

### Example 131, 1,1′-(2,2′-((3R,3′R,6R,6′R)-(((2S,2'S)-(1,4-diazepane-1,4-dicarbonyl)bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)

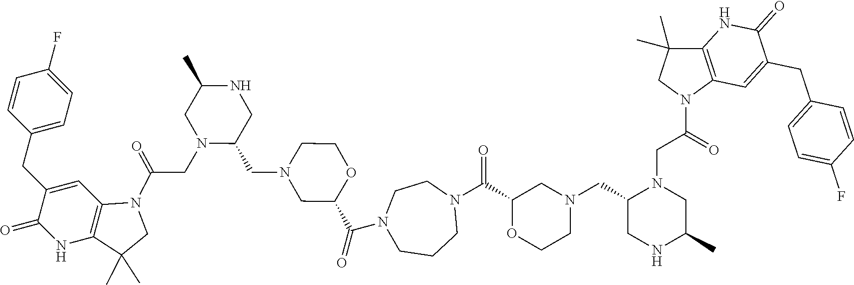 [SMILES: C[C@@H]1CN(CC(=O)N2CC(C)(C)c3[nH]c(=O)c(Cc4ccc(F)cc4)cc32)[C@@H](CN2CCO[C@H](C(=O)N3CCCN(C(=O)[C@@H]4CN(C[C@H]5CN[C@H](C)CN5CC(=O)N5CC(C)(C)c6[nH]c(=O)c(Cc7ccc(F)cc7)cc65)CCO4)CC3)C2)CN1]

LCMS (ES, m/z): 588.3 [M/2+H]⁺

¹H NMR (400 MHz, METHANOL-d₄) δ=8.14 (br s, 2H), 7.32-7.16 (m, 4H), 7.07-6.91 (m, 4H), 4.60-4.18 (m, 2H), 4.01-3.84 (m, 6H), 3.83-3.64 (m, 13H), 3.64-3.35 (m, 9H), 3.17 (br d, J=14.4 Hz, 7H), 3.10-2.95 (m, 7H), 2.94-2.27 (m, 6H), 1.88-1.61 (m, 2H), 1.47-1.35 (m, 12H), 1.29 (br d, J=6.5 Hz, 6H).

### Example 132, (S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-((1-((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carbonyl)piperidin-4-yl)methyl)-N-methylmorpholine-2-carboxamide

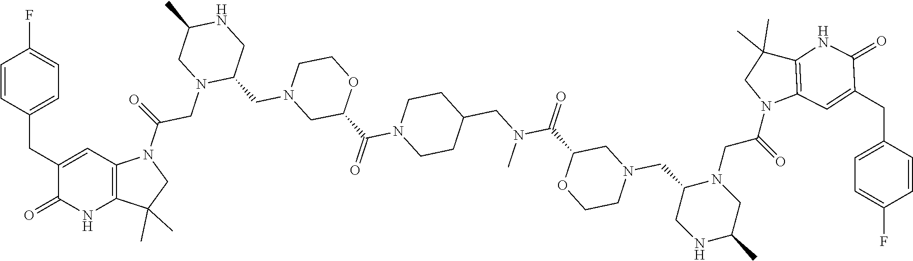 [SMILES: C[C@@H]1CN(CC(=O)N2CC(C)(C)c3[nH]c(=O)c(Cc4ccc(F)cc4)cc32)[C@@H](CN2CCO[C@H](C(=O)N(C)CC3CCN(C(=O)[C@@H]4CN(C[C@H]5CN[C@H](C)CN5CC(=O)N5CC(C)(C)c6[nH]c(=O)c(Cc7ccc(F)cc7)cc65)CCO4)CC3)C2)CN1]

LCMS (ES, m/z): 602.3 [M/2+H]⁺

¹H NMR (400 MHz, METHANOL-d₄) δ=8.23-8.04 (m, 2H), 7.23 (br s, 4H), 7.07-6.95 (m, 4H), 4.45-4.23 (m, 2H), 3.95-3.86 (m, 4H), 3.83 (br d, J=10.4 Hz, 4H), 3.79-3.70 (m, 7H), 3.69-3.47 (m, 5H), 3.44-3.36 (m, 3H), 3.24 (br s, 2H), 3.20-2.91 (m, 15H), 2.91-2.72 (m, 4H), 2.70-2.11 (m, 6H), 1.87 (br s, 1H), 1.57 (br s, 1H), 1.46-1.35 (m, 12H), 1.32-1.26 (m, 6H), 1.16-0.85 (m, 2H).

### Example 133, (S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-((1-((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carbonyl)piperidin-4-yl)methyl)morpholine-2-carboxamide

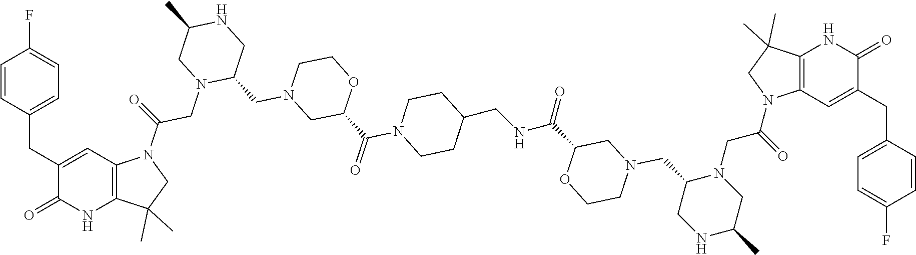 [SMILES: C[C@@H]1CN(CC(=O)N2CC(C)(C)c3[nH]c(=O)c(Cc4ccc(F)cc4)cc32)[C@@H](CN2CCO[C@H](C(=O)NCC3CCN(C(=O)[C@@H]4CN(C[C@H]5CN[C@H](C)CN5CC(=O)N5CC(C)(C)c6[nH]c(=O)c(Cc7ccc(F)cc7)cc65)CCO4)CC3)C2)CN1]

LCMS (ES, m/z): 595.3 [M/2+H]⁺

¹H NMR (400 MHz, METHANOL-d₄) δ=8.21-8.01 (m, 3H), 7.29-7.17 (m, 4H), 7.07-6.93 (m, 4H), 4.33 (br d, J=12.9 Hz, 1H), 4.06 (br s, 2H), 3.97-3.89 (m, 3H), 3.88-3.74 (m, 11H), 3.74-3.52 (m, 5H), 3.41 (br s, 3H), 3.26 (br s, 2H), 3.23-2.89 (m, 14H), 2.82 (br s, 3H), 2.67-2.35 (m, 4H), 1.67 (br d, J=12.9 Hz, 3H), 1.49-1.34 (m, 12H), 1.29 (br d, J=5.5 Hz, 6H), 1.22-0.87 (m, 2H).

### Example 134, (S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-(2-(4-((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carbonyl)piperazin-1-yl)ethyl)morpholine-2-carboxamide

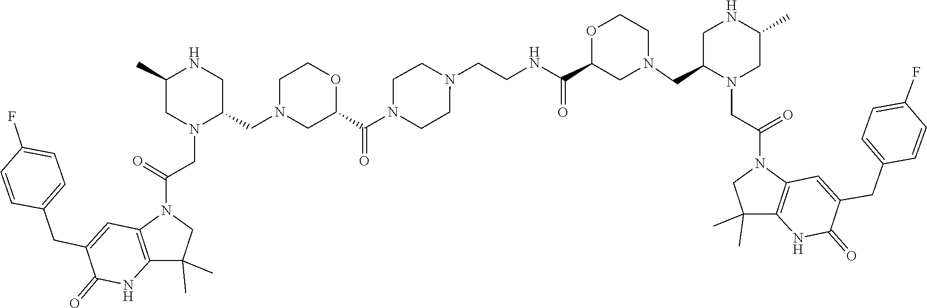 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCN3CCN(C(=O)[C@@H]4CN(C[C@H]5CN[C@H](C)CN5CC(=O)N5CC(C)(C)c6[nH]c(=O)c(Cc7ccc(F)cc7)cc65)CCO4)CC3)C2)CN1]

LCMS (ES, m/z): 602.8 [M/2+H]⁺

¹H NMR (400 MHz, METHANOL-d₄) δ=8.14 (s, 2H), 7.24 (td, J=6.1, 8.2 Hz, 4H), 7.00 (t, J=8.8 Hz, 4H), 4.04 (br dd, J=10.9, 18.7 Hz, 3H), 3.98-3.52 (m, 25H), 3.50-3.35 (m, 5H), 3.26 (br s, 6H), 3.21-2.91 (m, 12H), 2.89-2.47 (m, 3H), 1.41 (dd, J=3.4, 8.2 Hz, 12H), 1.29 (br d, J=5.8 Hz, 6H).

### Example 135, (S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-(3-((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamido)propyl)-N-methylmorpholine-2-carboxamide

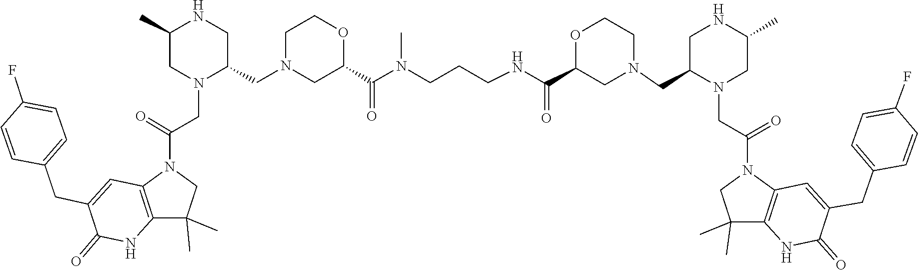 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCCN(C)C(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 582.3 [M/2+H]⁺

¹H NMR (400 MHz, METHANOL-d₄) δ=8.16-8.00 (m, 2H), 7.22 (br dd, J=5.1, 8.0 Hz, 4H), 7.00 (br t, J=8.6 Hz, 4H), 4.10-3.88 (m, 5H), 3.86 (br s, 2H), 3.84-3.73 (m, 9H), 3.72-3.52 (m, 4H), 3.51-3.35 (m, 4H), 3.28-3.05 (m, 13H), 3.04-2.89 (m, 6H), 2.86 (s, 2H), 2.80-2.62 (m, 2H), 2.53 (br s, 2H), 1.88-1.63 (m, 2H), 1.46-1.35 (m, 12H), 1.29 (br d, J=6.4 Hz, 6H).

### Example 136, (2S,2'S)-N,N′-(pentane-1,5-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

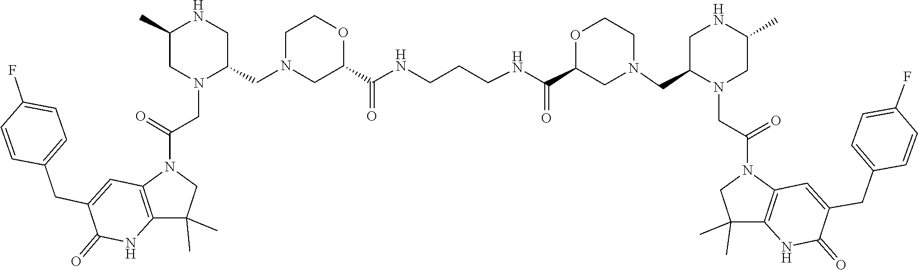 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCCNC(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 589.3 [M/2+H]⁺

¹H NMR (400 MHz, METHANOL-d₄) δ=8.12 (s, 2H), 7.89 (br s, 1H), 7.29-7.18 (m, 4H), 7.00 (t, J=8.7 Hz, 4H), 4.00-3.66 (m, 16H), 3.62 (br s, 2H), 3.48 (br s, 3H), 3.28-2.95 (m, 16H), 2.86 (br s, 4H), 2.52 (br s, 2H), 2.28 (br s, 3H), 1.54-1.44 (m, 4H), 1.39 (d, J=5.4 Hz, 12H), 1.28 (br d, J=6.4 Hz, 8H).

### Example 137, (2S,2'S)-N,N′-(2-hydroxypropane-1,3-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

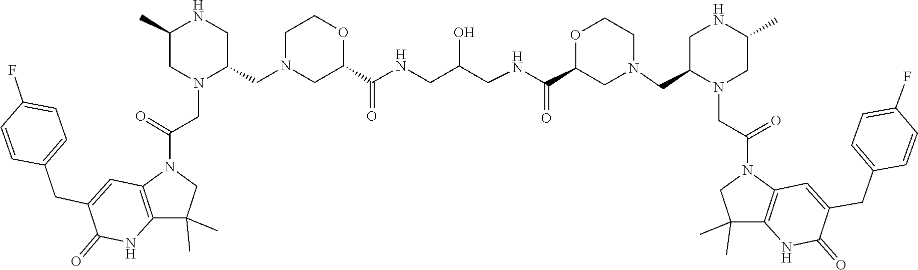 [SMILES: C[C@@H]1CN(CC(=O)N2CC(C)(C)c3[nH]c(=O)c(Cc4ccc(F)cc4)cc32)[C@@H](CN2CCO[C@H](C(=O)NCC(O)CNC(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 583.3 [M/2+H]P

¹H NMR (400 MHz, METHANOL-d₄) δ=8.08 (s, 2H), 7.91 (br s, 2H), 7.23 (dd, J=5.7, 7.8 Hz, 4H), 7.01 (t, J=8.8 Hz, 4H), 4.01 (br s, 2H), 3.95-3.86 (m, 4H), 3.79 (br t, J=17.8 Hz, 11H), 3.68 (br d, J=17.8 Hz, 3H), 3.60-3.43 (m, 3H), 3.38 (br s, 3H), 3.27 (br d, J=12.8 Hz, 6H), 3.13-3.02 (m, 5H), 2.94 (br d, J=11.4 Hz, 5H), 2.70 (br s, 2H), 2.49 (br s, 3H), 1.39 (br d, J=8.0 Hz, 12H), 1.28 (d, J=6.5 Hz, 6H).

### Example 138, (2S,2'S)-N,N′-(oxybis(ethane-2,1-diyl))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

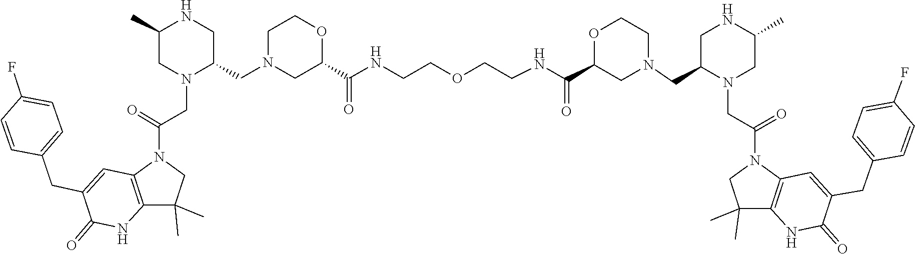 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCOCCNC(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 590.3 [M/2+H]⁺

¹H NMR (400 MHz, METHANOL-d₄) δ=8.08 (br s, 2H), 7.91 (br s, 1H), 7.26-7.20 (m, 4H), 7.01 (br t, J=8.6 Hz, 4H), 4.10-3.82 (m, 10H), 3.80 (br s, 6H), 3.77-3.60 (m, 6H), 3.52 (br s, 8H), 3.43 (br s, 4H), 3.11 (br s, 7H), 2.94 (br s, 5H), 2.80-2.31 (m, 4H), 1.40 (br d, J=7.0 Hz, 12H), 1.29 (br s, 6H).

### Example 139, (2S,2'S)-N,N′-(2,2-dimethylpropane-1,3-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

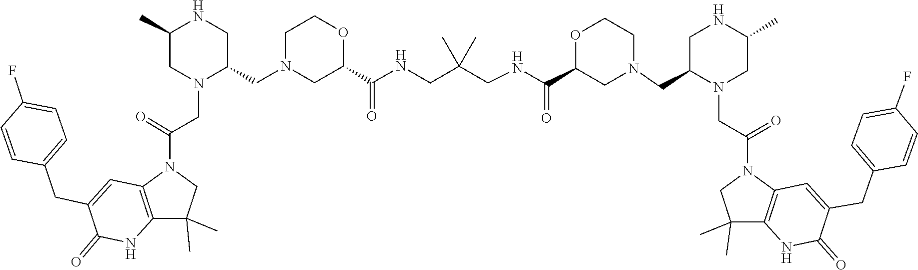 [SMILES: C[C@@H]1CN(CC(=O)N2CC(C)(C)c3[nH]c(=O)c(Cc4ccc(F)cc4)cc32)[C@@H](CN2CCO[C@H](C(=O)NCC(C)(C)CNC(=O)[C@@H]3CN(C[C@H]4CN[C@H](C)CN4CC(=O)N4CC(C)(C)c5[nH]c(=O)c(Cc6ccc(F)cc6)cc54)CCO3)C2)CN1]

LCMS (ES, m/z): 589.3 [M/2+H]⁺

¹H NMR (400 MHz, METHANOL-d₄) δ=8.12 (br s, 4H), 7.28-7.16 (m, 4H), 7.00 (t, J=8.5 Hz, 4H), 3.94 (br s, 2H), 3.89-3.77 (m, 12H), 3.76-3.65 (m, 3H), 3.62 (br d, J=11.5 Hz, 2H), 3.48 (br s, 3H), 3.27-3.16 (m, 4H), 3.15-3.04 (m, 5H), 2.95 (br s, 5H), 2.85 (br d, J=11.0 Hz, 4H), 2.49 (br s, 2H), 2.37-2.08 (m, 4H), 1.38 (br d, J=5.8 Hz, 12H), 1.28 (br d, J=6.4 Hz, 6H), 0.81 (br d, J=2.6 Hz, 6H).

### Example 140, (2S,2'S)-N,N′-(1,3-phenylene)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

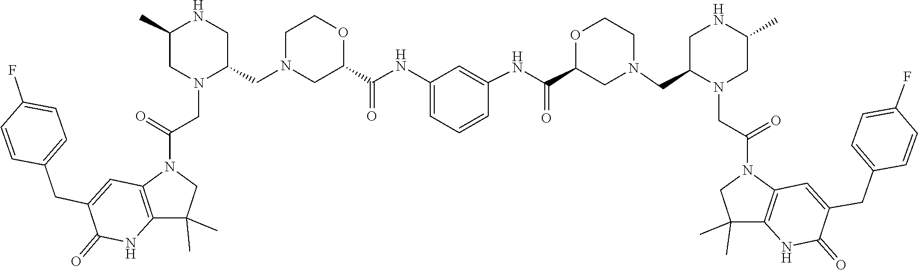 [SMILES: C[C@@H]1CN(CC(=O)N2CC(C)(C)c3[nH]c(=O)c(Cc4ccc(F)cc4)cc32)[C@@H](CN2CCO[C@H](C(=O)Nc3cccc(NC(=O)[C@@H]4CN(C[C@H]5CN[C@H](C)CN5CC(=O)N5CC(C)(C)c6[nH]c(=O)c(Cc7ccc(F)cc7)cc65)CCO4)c3)C2)CN1]

LCMS (ES, m/z): 592.3 [M/2+H]⁺

¹H NMR (400 MHz, METHANOL-d₄) δ=8.14 (s, 2H), 7.88 (br s, 1H), 7.36-7.20 (m, 7H), 6.98 (t, J=8.8 Hz, 4H), 3.96 (br s, 4H), 3.89-3.73 (m, 12H), 3.63-3.44 (m, 5H), 3.35 (br s, 3H), 3.19-3.01 (m, 7H), 3.00-2.69 (m, 6H), 2.49-2.21 (m, 5H), 1.37 (s, 12H), 1.28 (br d, J=6.5 Hz, 6H).

### Example 141, (2S,2'S)-N,N′-(2-methyl-1,3-phenylene)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

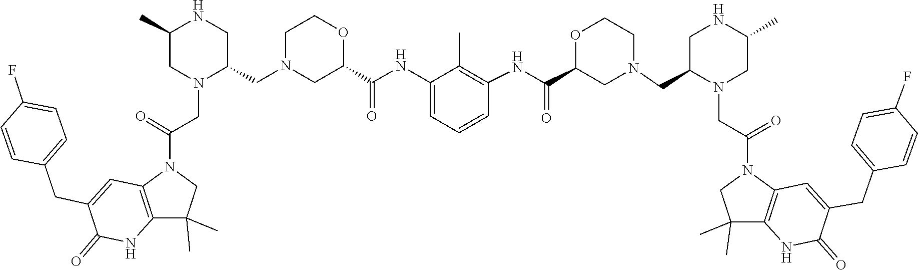 [SMILES: Cc1c(NC(=O)[C@@H]2CN(C[C@H]3CN[C@H](C)CN3CC(=O)N3CC(C)(C)c4[nH]c(=O)c(Cc5ccc(F)cc5)cc43)CCO2)cccc1NC(=O)[C@@H]1CN(C[C@H]2CN[C@H](C)CN2CC(=O)N2CC(C)(C)c3[nH]c(=O)c(Cc4ccc(F)cc4)cc32)CCO1]

LCMS (ES, m/z): 599.3 [M/2+H]P

¹H NMR (400 MHz, METHANOL-d₄) δ=8.17 (s, 2H), 7.33-7.15 (m, 7H), 6.99 (t, J=8.8 Hz, 4H), 4.08-3.93 (m, 4H), 3.92-3.84 (m, 4H), 3.84-3.78 (m, 6H), 3.77-3.68 (m, 3H), 3.67-3.43 (m, 5H), 3.27 (br s, 4H), 3.20-2.93 (m, 7H), 2.92-2.79 (m, 4H), 2.59-2.26 (m, 5H), 1.93 (s, 3H), 1.38 (d, J=14.6 Hz, 12H), 1.29 (d, J=6.4 Hz, 6H).

### Example 142, 1-[6-(4-fluorophenoxy)-3,3-dimethyl-2H-pyrrolo[3,2-b]pyridin-1-yl]-2-[(2R,5R)-2-[[(2S)-2-[[3-[[(2S)-4-[[(2R,5R)-1-[2-[6-(4-fluorophenoxy)-3,3-dimethyl-2H-pyrrolo[3,2-b]pyridin-1-yl]-2-oxo-ethyl]-5-methyl-piperazin-2-yl]methyl]morpholin-2-yl]methoxy]phenoxy]methyl]morpholin-4-yl]methyl]-5-methyl-piperazin-1-yl]ethanone

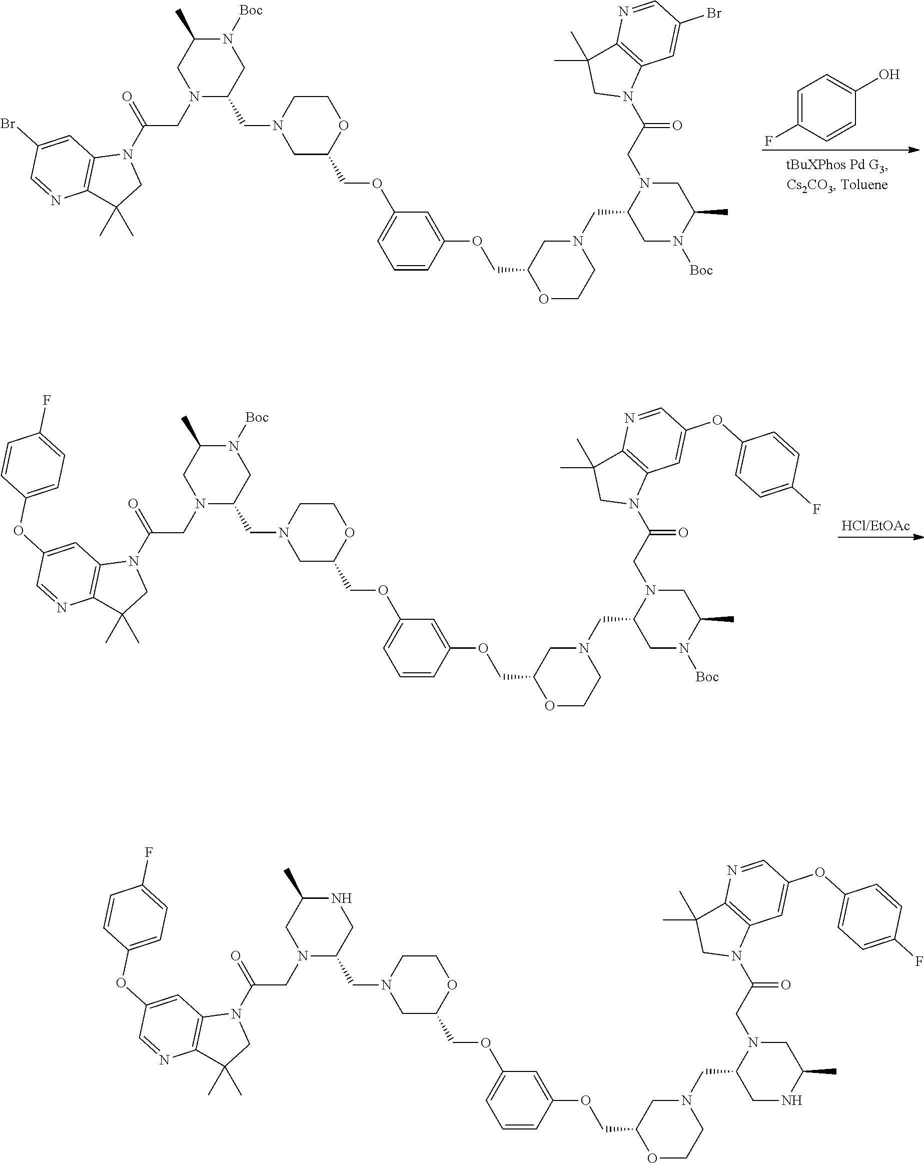

LCMS (ES, m/z): 565.3 [M/2+H]⁺

¹H NMR (400 MHz, METHANOL-d₄) δ=7.99-7.95 (m, 2H), 7.95-7.92 (m, 2H), 7.18-7.11 (m, 2H), 7.11-7.07 (m, 3H), 7.06-6.96 (m, 4H), 6.50 (dd, J=2.3, 8.3 Hz, 2H), 6.39 (s, 1H), 4.08-3.86 (m, 16H), 3.82 (br s, 2H), 3.76-3.57 (m, 5H), 3.45 (br s, 3H), 3.27-3.15 (m, 5H), 3.12-2.97 (m, 5H), 2.93-2.59 (m, 6H), 1.41 (d, J=2.6 Hz, 12H), 1.32 (br d, J=6.6 Hz, 6H)

### Example 143, 1-[6-(4-fluoroanilino)-3,3-dimethyl-2H-pyrrolo[3,2-b]pyridin-1-yl]-2-[(2R,5R)-2-[[(2S)-2-[[3-[[(2S)-4-[[(2R,5R)-1-[2-[6-(4-fluoroanilino)-3,3-dimethyl-2H-pyrrolo[3,2-b]pyridin-1-yl]-2-oxo-ethyl]-5-methyl-piperazin-2-yl]methyl]morpholin-2-yl]methoxy]phenoxy]methyl]morpholin-4-yl]methyl]-5-methyl-piperazin-1-yl]ethenone

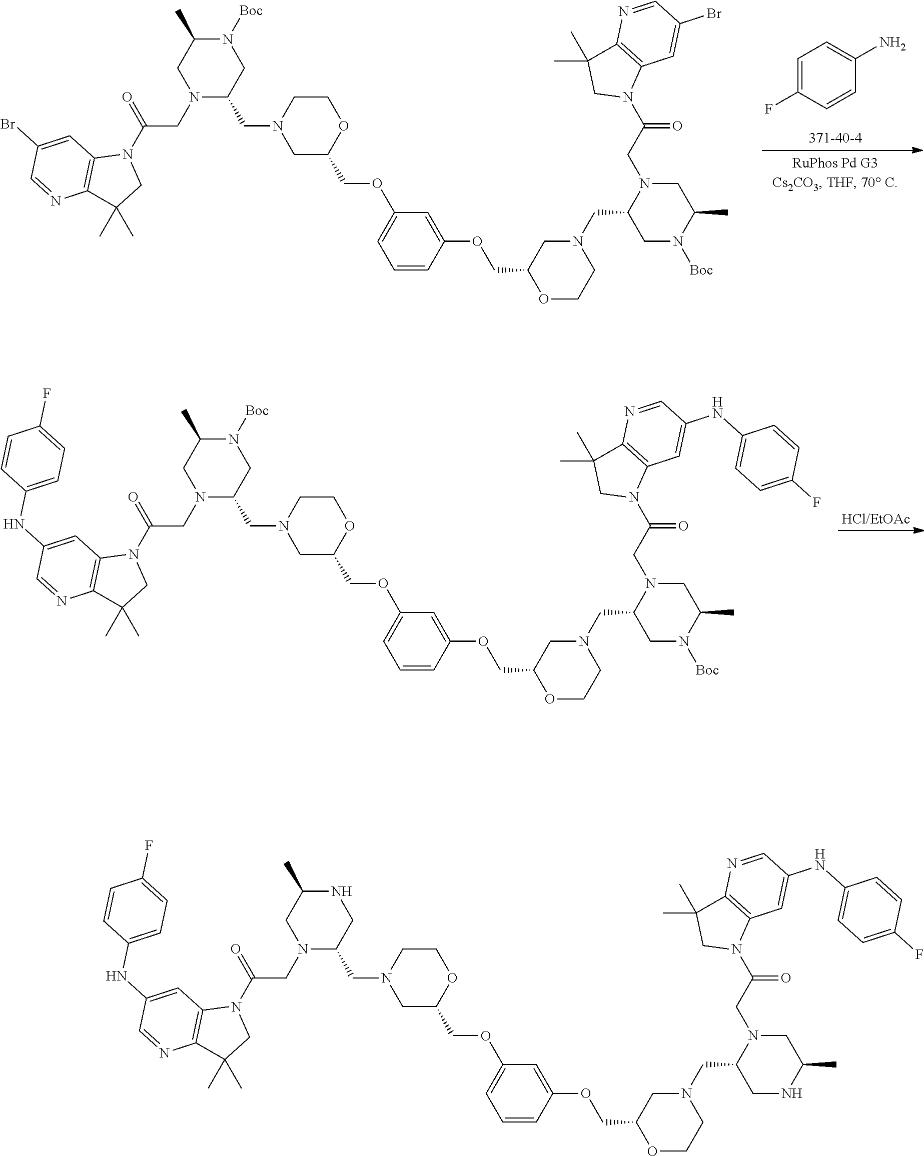

LCMS (ES, m/z): 564.5 [M/2+H]⁺

¹H NMR (400 MHz, METHANOL-d₄) δ=8.29-8.25 (m, 2H), 7.84-7.81 (m, 2H), 7.14-6.98 (m, 9H), 6.44 (dd, J=1.8, 8.1 Hz, 2H), 6.32 (s, 1H), 4.07-3.99 (m, 4H), 3.98-3.79 (m, 13H), 3.75-3.63 (m, 4H), 3.51-3.40 (m, 3H), 3.26-3.16 (m, 5H), 3.15-2.96 (m, 7H), 2.92-2.51 (m, 6H), 1.44-1.39 (m, 12H), 1.33 (br d, J=6.5 Hz, 6H)

Compound 144 was prepared according to the same procedure as Compound 143, replacing 4-fluoroaniline with 4-fluoro-N-methylaniline. Compound 144 was found to have characterizing data as set forth below.

### Example 144, 2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(6-((4-fluorophenyl)(methyl)amino)-3,3-dimethyl-2,3-dihydro-1H-pyrrolo[3,2-b]pyridin-1-yl)ethan-1-one)

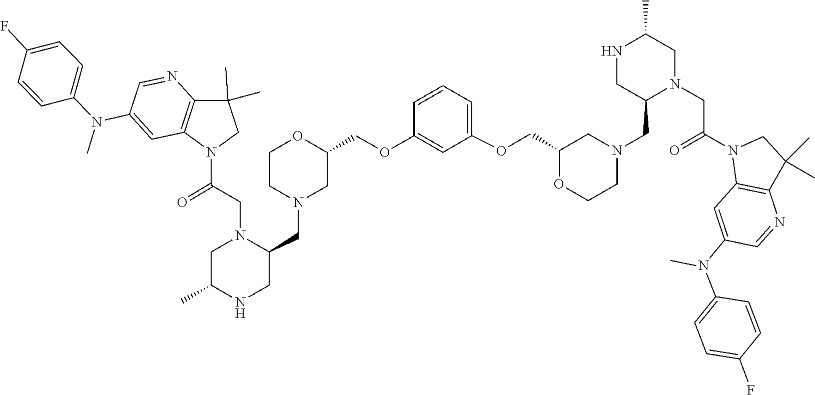 [SMILES: C[C@@H]1CN(CC(=O)N2CC(C)(C)c3ncc(N(C)c4ccc(F)cc4)cc32)[C@@H](CN2CCO[C@H](COc3cccc(OC[C@@H]4CN(C[C@H]5CN[C@H](C)CN5CC(=O)N5CC(C)(C)c6ncc(N(C)c7ccc(F)cc7)cc65)CCO4)c3)C2)CN1]

LCMS (ES, m/z): 578.5 [M/2+H]⁺

¹H NMR (400 MHz, METHANOL-d₄) δ=8.09 (d, J=2.3 Hz, 2H), 7.75 (d, J=2.4 Hz, 2H), 7.25-7.13 (m, 9H), 6.53 (dd, J=2.1, 8.3 Hz, 2H), 6.44 (s, 1H), 4.17-4.09 (m, 4H), 4.07-3.97 (m, 6H), 3.96 (s, 6H), 3.86-3.70 (m, 4H), 3.62-3.48 (m, 6H), 3.47-3.33 (m, 4H), 3.29 (s, 6H), 3.27-3.17 (m, 4H), 3.16-2.95 (m, 8H), 1.49 (s, 12H), 1.33 (d, J=6.7 Hz, 6H).

Examples 145-190 were prepared according to the same general procedures and coupling/deprotection techniques as disclosed in Examples 1-144. The compounds of Examples 145-190 were found to have characterizing data as set forth below.

### Example 145, 2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(6-(4-fluorobenzyl)-3,3-dimethyl-2,3-dihydro-1H-pyrrolo[3,2-b]pyridin-1-yl)ethan-1-one)

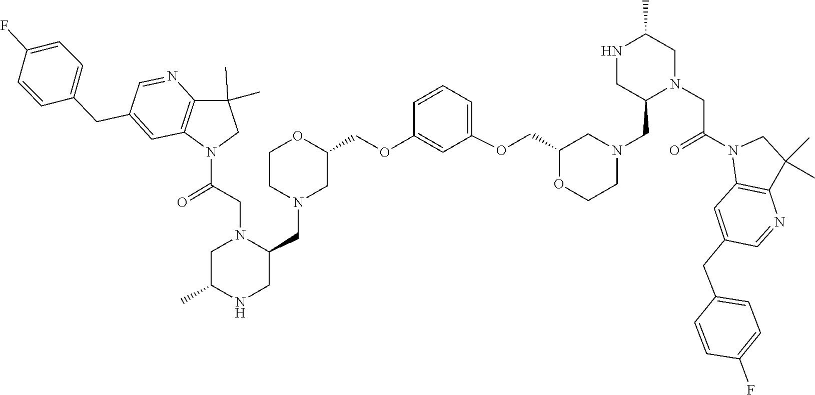 [SMILES: C[C@@H]1CN(CC(=O)N2CC(C)(C)c3ncc(Cc4ccc(F)cc4)cc32)[C@@H](CN2CCO[C@H](COc3cccc(OC[C@@H]4CN(C[C@H]5CN[C@H](C)CN5CC(=O)N5CC(C)(C)c6ncc(Cc7ccc(F)cc7)cc65)CCO4)c3)C2)CN1]

LCMS (ES, m/z): 563.4 [M/2+H]⁺

¹H NMR (400 MHz, METHANOL-d₄) δ=8.20 (d, J=1.8 Hz, 2H), 8.11 (d, J=1.3 Hz, 2H), 7.21-7.10 (m, 5H), 6.98 (t, J=8.8 Hz, 4H), 6.49 (dd, J=2.2, 8.3 Hz, 2H), 6.39 (s, 1H), 4.14-3.82 (m, 20H), 3.74 (br s, 5H), 3.55-3.33 (m, 6H), 3.22 (br d, J=14.3 Hz, 4H), 3.15-2.99 (m, 5H), 2.99-2.58 (m, 6H), 1.41 (d, J=3.5 Hz, 12H), 1.32 (d, J=6.6 Hz, 6H).

### Example 146, 2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(3,3-dimethyl-6-(pyridin-3-ylmethyl)-2,3-dihydro-1H-pyrrolo[3,2-b]pyridin-1-yl)ethan-1-one)

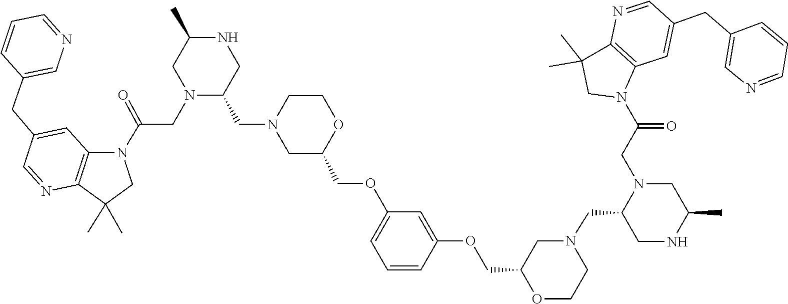 [SMILES: C[C@@H]1CN(CC(=O)N2CC(C)(C)c3ncc(Cc4cccnc4)cc32)[C@@H](CN2CCO[C@H](COc3cccc(OC[C@@H]4CN(C[C@H]5CN[C@H](C)CN5CC(=O)N5CC(C)(C)c6ncc(Cc7cccnc7)cc65)CCO4)c3)C2)CN1]

LCMS (ES, m/z): 546.4 [M/2+H]⁺

¹H NMR (400 MHz, METHANOL-d₄) δ=8.76-8.55 (m, 4H), 8.35-8.22 (m, 4H), 8.17 (d, J=1.8 Hz, 2H), 7.87-7.76 (m, 2H), 7.12 (t, J=8.3 Hz, 1H), 6.48-6.43 (m, 2H), 6.32-6.28 (m, 1H), 4.22-4.15 (m, 4H), 4.14-4.03 (m, 6H), 4.03-3.90 (m, 10H), 3.89-3.75 (m, 4H), 3.56-3.39 (m, 6H), 3.38-3.33 (m, 2H), 3.28-3.22 (m, 2H), 3.16-3.02 (m, 6H), 2.92 (br d, J=10.6 Hz, 4H), 1.46-1.36 (m, 12H), 1.35-1.27 (m, 6H).

### Example 147, 2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(6-(4-chlorobenzyl)-3,3-dimethyl-2,3-dihydro-1H-pyrrolo[3,2-b]pyridin-1-yl)ethan-1-one)

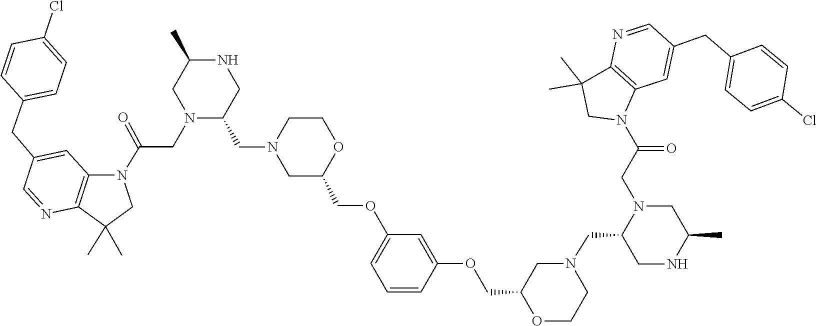 [SMILES: C[C@@H]1CN(CC(=O)N2CC(C)(C)c3ncc(Cc4ccc(Cl)cc4)cc32)[C@@H](CN2CCO[C@H](COc3cccc(OC[C@@H]4CN(C[C@H]5CN[C@H](C)CN5CC(=O)N5CC(C)(C)c6ncc(Cc7ccc(Cl)cc7)cc65)CCO4)c3)C2)CN1]

LCMS (ES, m/z): 579.4 [M/2+H]P

¹H NMR (400 MHz, METHANOL-d₄) δ=8.22 (d, J=1.5 Hz, 2H), 7.97 (d, J=1.6 Hz, 2H), 7.40-6.97 (m, 9H), 6.46-6.20 (m, 3H), 4.72-4.52 (m, 3H), 4.01-3.60 (m, 18H), 3.52-3.40 (m, 2H), 3.35 (br s, 1H), 3.13-2.49 (m, 18H), 2.12-1.84 (m, 6H), 1.37 (d, J=9.8 Hz, 12H), 1.09 (d, J=6.4 Hz, 6H).

### Example 148, 2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(3,3-dimethyl-6-(4-methylbenzyl)-2,3-dihydro-1H-pyrrolo[3,2-b]pyridin-1-yl)ethan-1-one)

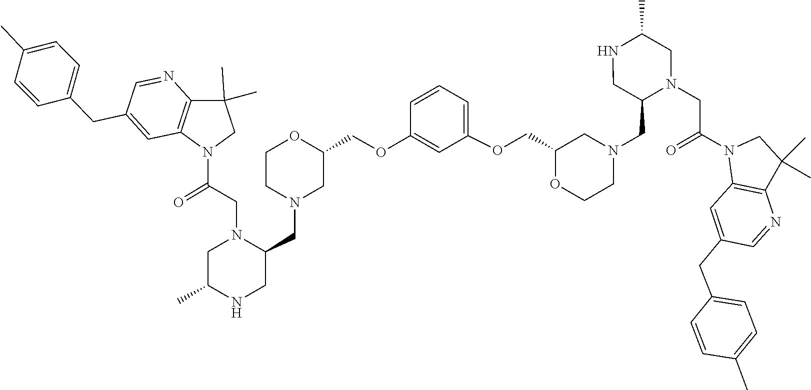

LCMS (ES, m/z): 559.5 [M/2+H]⁺

¹H NMR (400 MHz, METHANOL-d₄) δ=8.41 (br s, 3H), 8.21 (d, J=1.4 Hz, 2H), 8.00 (d, J=1.4 Hz, 2H), 7.14-6.99 (m, 9H), 6.41 (dd, J=2.1, 8.3 Hz, 2H), 6.30 (s, 1H), 3.95-3.89 (m, 3H), 3.87 (s, 5H), 3.86-3.81 (m, 2H), 3.81-3.73 (m, 4H), 3.69 (br dd, J=4.5, 10.0 Hz, 2H), 3.63 (br d, J=11.0 Hz, 2H), 3.56-3.44 (m, 4H), 3.29-3.19 (m, 5H), 3.18-3.07 (m, 2H), 3.06-2.95 (m, 2H), 2.87-2.74 (m, 6H), 2.74-2.60 (m, 3H), 2.26 (s, 6H), 2.17 (br d, J=12.4 Hz, 2H), 2.02-1.89 (m, 4H), 1.37 (d, J=4.1 Hz, 12H), 1.28 (br d, J=6.3 Hz, 6H).

### Example 149, 2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(3,3-dimethyl-6-(4-(trifluoromethyl)benzyl)-2,3-dihydro-1H-pyrrolo[3,2-b]pyridin-1-yl)ethan-1-one)

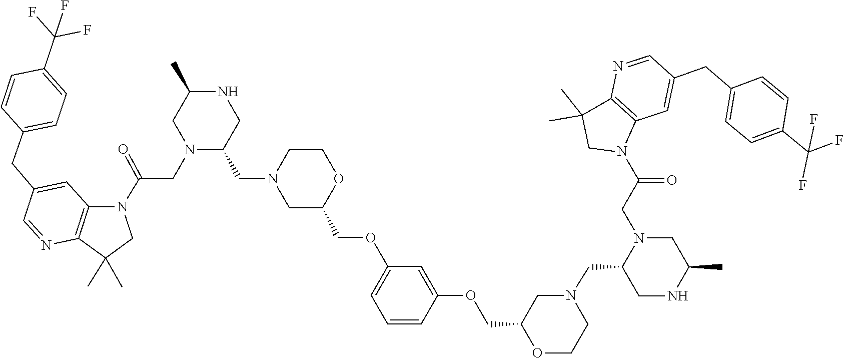 [SMILES: C[C@@H]1CN(CC(=O)N2CC(C)(C)c3ncc(Cc4ccc(C(F)(F)F)cc4)cc32)[C@@H](CN2CCO[C@H](COc3cccc(OC[C@@H]4CN(C[C@H]5CN[C@H](C)CN5CC(=O)N5CC(C)(C)c6ncc(Cc7ccc(C(F)(F)F)cc7)cc65)CCO4)c3)C2)CN1]

LCMS (ES, m/z): 613.4 [M/2+H]⁺

¹H NMR (400 MHz, METHANOL-d₄) δ=10.60-10.30 (m, 1H), 10.18 (s, 2H), 10.04 (s, 2H), 9.54 (br d, J=8.1 Hz, 4H), 9.35 (br d, J=8.0 Hz, 4H), 9.08 (t, J=8.3 Hz, 1H), 8.45-8.22 (m, 3H), 6.65-6.51 (m, 4H), 5.94-5.56 (m, 14H), 5.55-5.41 (m, 4H), 5.26-4.96 (m, 10H), 4.85-4.58 (m, 8H), 4.15 (br d, J=12.3 Hz, 2H), 4.01-3.85 (m, 4H), 3.36 (d, J=5.1 Hz, 12H), 3.29-3.23 (m, 6H).

### Example 150, 2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(6-benzyl-3,3-dimethyl-2,3-dihydro-1H-pyrrolo[3,2-b]pyridin-1-yl)ethan-1-one)

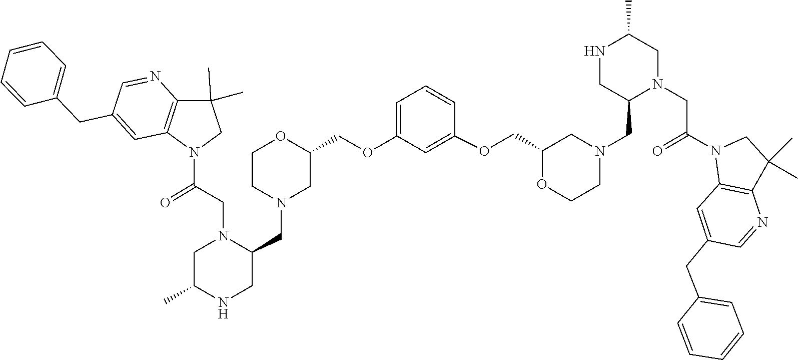 [SMILES: C[C@@H]1CN(CC(=O)N2CC(C)(C)c3ncc(Cc4ccccc4)cc32)[C@@H](CN2CCO[C@H](COc3cccc(OC[C@@H]4CN(C[C@H]5CN[C@H](C)CN5CC(=O)N5CC(C)(C)c6ncc(Cc7ccccc7)cc65)CCO4)c3)C2)CN1]

LCMS (ES, m/z): 545.5 [M/2+H]⁺

¹H NMR (400 MHz, METHANOL-d₄) δ=8.55 (br s, 1H), 8.23 (d, J=1.5 Hz, 2H), 8.00 (d, J=1.6 Hz, 2H), 7.28-7.22 (m, 4H), 7.21-7.04 (m, 7H), 6.40 (dd, J=2.1, 8.3 Hz, 2H), 6.29 (s, 1H), 3.97-3.89 (m, 6H), 3.85 (br d, J=10.5 Hz, 2H), 3.82-3.72 (m, 6H), 3.66 (td, J=5.0, 9.9 Hz, 4H), 3.46 (br s, 2H), 3.29-3.23 (m, 3H), 3.14-2.99 (m, 4H), 2.97-2.71 (m, 9H), 2.70-2.57 (m, 4H), 2.10 (br d, J=11.6 Hz, 2H), 1.97-1.86 (m, 4H), 1.37 (d, J=6.4 Hz, 12H), 1.16 (d, J=6.4 Hz, 6H).

### Example 151, 2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(6-(4-fluoro-2-methoxybenzyl)-3,3-dimethyl-2,3-dihydro-1H-pyrrolo[3,2-b]pyridin-1-yl)ethan-1-one)

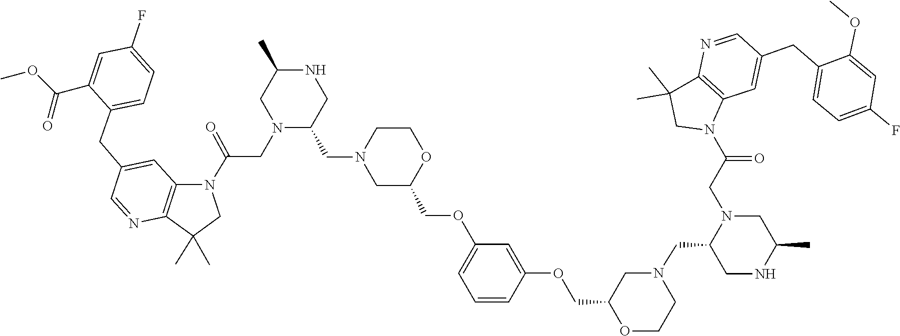 [SMILES: COC(=O)c1cc(F)ccc1Cc1cnc2c(c1)N(C(=O)CN1C[C@@H](C)NC[C@@H]1CN1CCO[C@H](COc3cccc(OC[C@@H]4CN(C[C@H]5CN[C@H](C)CN5CC(=O)N5CC(C)(C)c6ncc(Cc7ccc(F)cc7OC)cc65)CCO4)c3)C1)CC2(C)C]

LCMS (ES, m/z): 593.4 [M/2+H]⁺

¹H NMR (400 MHz, METHANOL-d₄) δ=8.23 (s, 2H), 8.11 (s, 2H), 7.17-7.06 (m, 3H), 6.71 (dd, J=2.4, 11.0 Hz, 2H), 6.59 (dt, J=2.4, 8.3 Hz, 2H), 6.48 (br d, J=8.3 Hz, 2H), 6.38 (br s, 1H), 4.11-3.95 (m, 10H), 3.92 (br s, 6H), 3.87 (br d, J=3.4 Hz, 4H), 3.85-3.67 (m, 12H), 3.57-3.39 (m, 5H), 3.34 (br s, 3H), 3.24 (br d, J=11.8 Hz, 4H), 3.08 (br dd, J=10.3, 14.1 Hz, 6H), 2.87 (br s, 2H), 1.41 (br d, J=4.9 Hz, 12H), 1.32 (d, J=6.5 Hz, 6H).

### Example 152, dimethyl 6,6′-(((2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(acetyl))bis(3,3-dimethyl-2,3-dihydro-1H-pyrrolo[3,2-b]pyridine-1,6-diyl))bis(methylene))bis(3-fluorobenzoate)

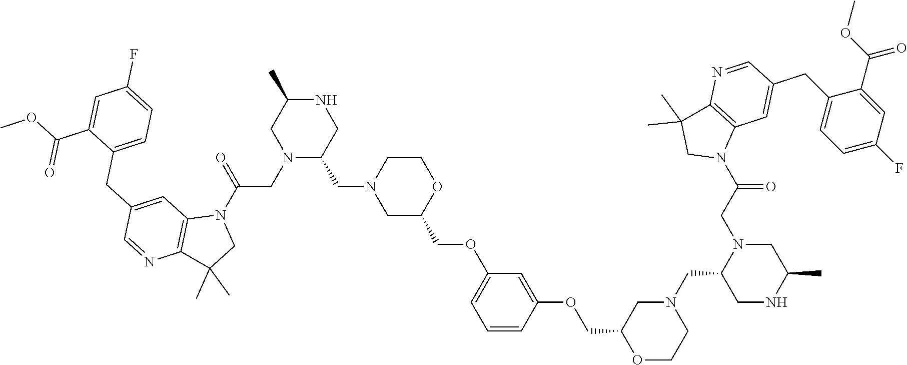 [SMILES: COC(=O)c1cc(F)ccc1Cc1cnc2c(c1)N(C(=O)CN1C[C@@H](C)NC[C@@H]1CN1CCO[C@H](COc3cccc(OC[C@@H]4CN(C[C@H]5CN[C@H](C)CN5CC(=O)N5CC(C)(C)c6ncc(Cc7ccc(F)cc7C(=O)OC)cc65)CCO4)c3)C1)CC2(C)C]

LCMS (ES, m/z): 621.4 [M/2+H]⁺

¹H NMR (400 MHz, METHANOL-d₄) δ=8.46 (br s, 2H), 8.14 (d, J=1.6 Hz, 2H), 7.97 (d, J=1.5 Hz, 2H), 7.59 (dd, J=2.8, 9.4 Hz, 2H), 7.38-7.30 (m, 2H), 7.28-7.18 (m, 2H), 7.09 (t, J=8.2 Hz, 1H), 6.40 (dd, J=2.1, 8.3 Hz, 2H), 6.29 (s, 1H), 4.37-4.26 (m, 4H), 3.94-3.88 (m, 3H), 3.85 (br d, J=6.5 Hz, 3H), 3.81 (s, 6H), 3.78 (br t, J=5.0 Hz, 3H), 3.75-3.62 (m, 5H), 3.54-3.42 (m, 4H), 3.29-3.17 (m, 6H), 3.16-3.06 (m, 2H), 3.04-2.96 (m, 2H), 2.86-2.63 (m, 8H), 2.20-2.13 (m, 2H), 2.02-1.89 (m, 4H), 1.36 (d, J=6.0 Hz, 12H), 1.27 (d, J=6.5 Hz, 6H)

### Example 153, 1-[6-[(4-bromophenyl)methyl]-3,3-dimethyl-2H-pyrrolo[3,2-b]pyridin-1-yl]-2-[(2R,5R)-2-[[(2S)-2-[[3-[[(2S)-4-[[(2R,5R)-1-[2-[6-[(4-bromophenyl)methyl]-3,3-dimethyl-2H-pyrrolo[3,2-b]pyridin-1-yl]-2-oxo-ethyl]-5-methyl-piperazin-2-yl]methyl]morpholin-2-yl]methoxy]phenoxy]methyl]morpholin-4-yl]methyl]-5-methyl-piperazin-1-yl]ethanone

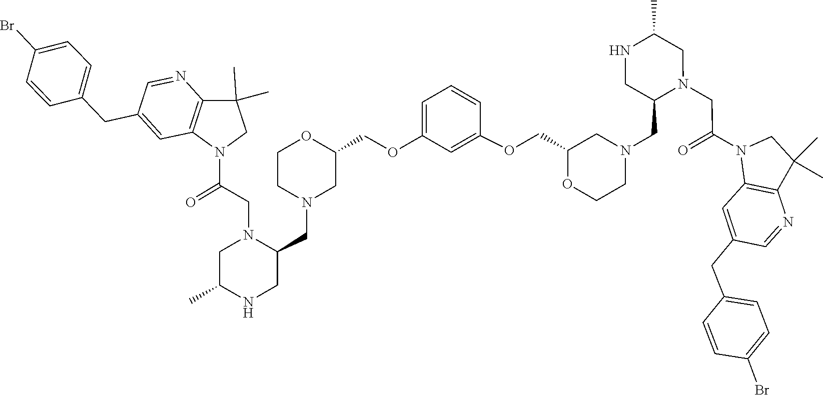 [SMILES: C[C@@H]1CN(CC(=O)N2CC(C)(C)c3ncc(Cc4ccc(Br)cc4)cc32)[C@@H](CN2CCO[C@H](COc3cccc(OC[C@@H]4CN(C[C@H]5CN[C@H](C)CN5CC(=O)N5CC(C)(C)c6ncc(Cc7ccc(Br)cc7)cc65)CCO4)c3)C2)CN1]

LCMS (ES, m/z): 624.4 [M/2+H]⁺

¹H NMR (400 MHz, METHANOL-d₄) δ=8.64-8.50 (m, 1H), 8.27-8.15 (m, 2H), 7.97 (s, 2H), 7.39 (br d, J=7.9 Hz, 4H), 7.27-6.97 (m, 5H), 6.40 (br d, J=8.1 Hz, 2H), 6.31-6.18 (m, 1H), 4.07-3.81 (m, 9H), 3.80-3.71 (m, 5H), 3.71-3.59 (m, 4H), 3.45 (br s, 2H), 3.17 (br s, 2H), 2.99 (br d, J=13.3 Hz, 5H), 2.87 (br t, J=10.1 Hz, 4H), 2.82-2.70 (m, 4H), 2.69-2.51 (m, 4H), 2.35 (s, 1H), 2.08 (br d, J=13.4 Hz, 2H), 1.92 (br t, J=11.0 Hz, 4H), 1.42-1.32 (m, 12H), 1.14 (br d, J=5.9 Hz, 6H)

### Example 154, 6-(4-fluorobenzyl)-1-(2-((2R,5R)-2-(((R)-4-(2-(1-(3-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-3-oxopropyl)-1H-1,2,3-triazol-4-yl)ethyl)-2-methylpiperazin-1-yl)methyl)-5-methylpiperazin-1-yl)acetyl)-3,3-dimethyl-1,2,3,4-tetrahydro-5H-pyrrolo[3,2-b]pyridin-5-one

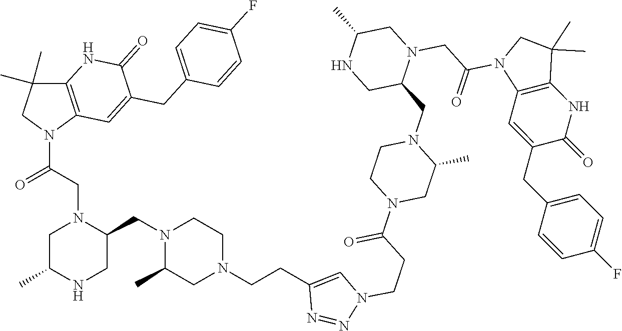 [SMILES: C[C@@H]1CN(CC(=O)N2CC(C)(C)c3[nH]c(=O)c(Cc4ccc(F)cc4)cc32)[C@@H](CN2CCN(CCc3cn(CCC(=O)N4CCN(C[C@H]5CN[C@H](C)CN5CC(=O)N5CC(C)(C)c6[nH]c(=O)c(Cc7ccc(F)cc7)cc65)[C@H](C)C4)nn3)C[C@H]2C)CN1]

LCMS (ES, m/z): 599.96 [M/2+H]⁺.

¹H NMR (400 MHz, Methanol-d₄) δ 8.13 (s, 1H), 7.95-7.86 (m, 2H), 7.23 (ddd, J=8.6, 5.4, 3.1 Hz, 4H), 7.04-6.93 (m, 4H), 4.80-4.72 (m, 1H), 4.04-3.95 (m, 2H), 3.92-3.76 (m, 11H), 3.78-3.59 (m, 4H), 3.59-3.33 (m, 10H), 3.31-3.16 (m, 6H), 3.17-2.97 (m, 8H), 2.90 (q, J=12.7 Hz, 3H), 2.63 (s, 2H), 2.31 (s, 1H), 1.45-1.39 (m, 13H), 1.36-1.21 (m, 10H), 1.17 (d, J=6.1 Hz, 3H).

### Example 155, 6-(4-fluorobenzyl)-1-(2-((2R,5R)-2-(((R)-4-((1-(2-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-2-oxoethyl)-1H-1,2,3-triazol-4-yl)methyl)-2-methylpiperazin-1-yl)methyl)-5-methylpiperazin-1-yl)acetyl)-3,3-dimethyl-1,2,3,4-tetrahydro-5H-pyrrolo[3,2-b]pyridin-5-one

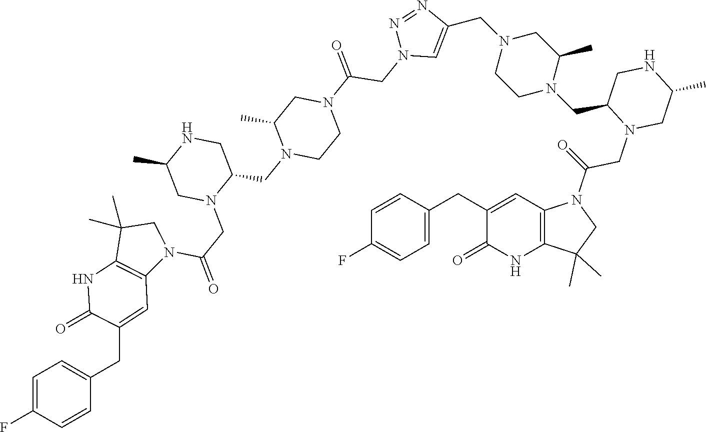 [SMILES: C[C@@H]1CN(CC(=O)N2CC(C)(C)c3[nH]c(=O)c(Cc4ccc(F)cc4)cc32)[C@@H](CN2CCN(Cc3cn(CC(=O)N4CCN(C[C@H]5CN[C@H](C)CN5CC(=O)N5CC(C)(C)c6[nH]c(=O)c(Cc7ccc(F)cc7)cc65)[C@H](C)C4)nn3)C[C@H]2C)CN1]

LCMS (ES, m/z): 585.95 [M/2+H]⁺

¹H NMR (400 MHz, Methanol-d₄) δ 8.10 (d, J=7.0 Hz, 2H), 7.98 (s, 1H), 7.26 (dd, J=8.5, 5.5 Hz, 4H), 7.03 (q, J=9.1 Hz, 4H), 5.61 (d, J=13.5 Hz, 2H), 4.30 (s, 2H), 4.06-3.95 (m, 1H), 3.93-3.79 (m, 10H), 3.79-3.61 (m, 4H), 3.61-3.45 (m, 4H), 3.43-3.33 (m, 5H), 3.28-2.31 (m, 18H), 1.43 (s, 3H), 1.42 (s, 6H), 1.40 (s, 4H), 1.38-1.30 (m, 4H), 1.28 (d, J=6.6 Hz, 3H), 1.25 (d, J=6.2 Hz, 1H), 1.22-1.12 (m, 3H).

### Example 156, 6-(4-fluorobenzyl)-1-(2-((2R,5R)-2-(((R)-4-((1-(3-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-3-oxopropyl)-1H-1,2,3-triazol-4-yl)methyl)-2-methylpiperazin-1-yl)methyl)-5-methylpiperazin-1-yl)acetyl)-3,3-dimethyl-1,2,3,4-tetrahydro-5H-pyrrolo[3,2-b]pyridin-5-one

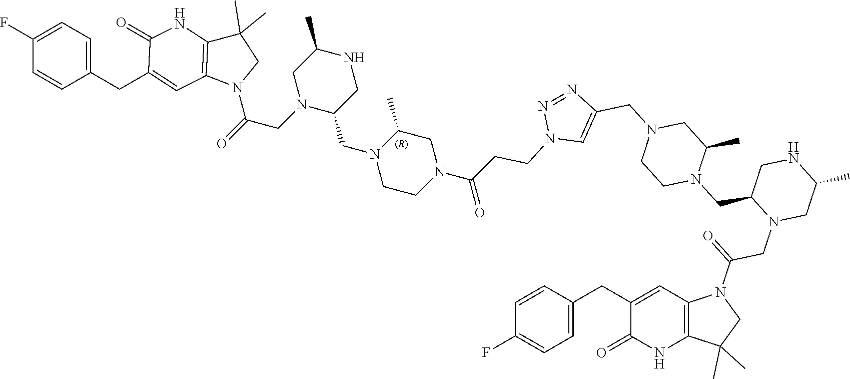

LCMS (ES, m/z): 592.85 [M/2+H]⁺

1H NMR (400 MHz, Methanol-d₄) δ 8.06 (d, J=57.2 Hz, 2H), 7.87 (s, 1H), 7.24 (dd, J=8.7, 5.3 Hz, 4H), 7.02 (dt, J=17.2, 8.7 Hz, 4H), 4.01-3.93 (m, 2H), 3.92-3.75 (m, 10H), 3.69 (d, J=17.0 Hz, 2H), 3.57-3.34 (m, 9H), 3.28-2.53 (m, 23H), 1.43 (s, 3H), 1.41 (d, J=3.5 Hz, 6H), 1.40 (s, 4H), 1.39-1.30 (m, 5H), 1.28 (d, J=6.5 Hz, 3H), 1.27-1.20 (m, 2H), 1.18 (d, J=5.1 Hz, 3H).

### Example 157, 6-(4-fluorobenzyl)-1-(2-((2R,5R)-2-(((R)-4-(2-(1-(4-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-4-oxobutyl)-1H-1,2,3-triazol-4-yl)ethyl)-2-methylpiperazin-1-yl)methyl)-5-methylpiperazin-1-yl)acetyl)-3,3-dimethyl-1,2,3,4-tetrahydro-5H-pyrrolo[3,2-b]pyridin-5-one

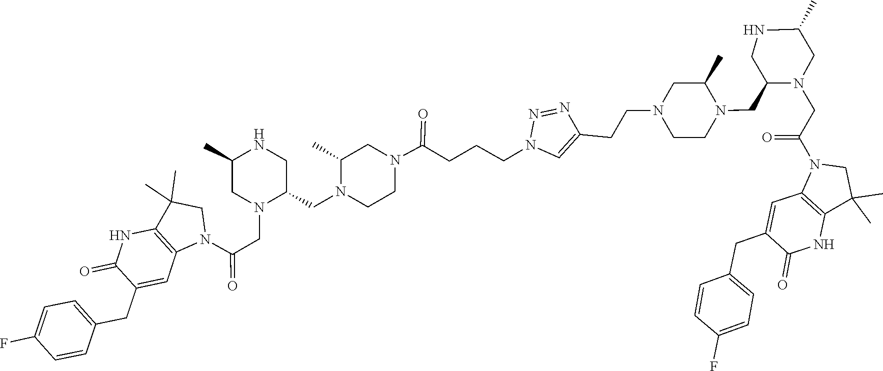 [SMILES: C[C@@H]1CN(CC(=O)N2CC(C)(C)c3[nH]c(=O)c(Cc4ccc(F)cc4)cc32)[C@@H](CN2CCN(CCc3cn(CCCC(=O)N4CCN(C[C@H]5CN[C@H](C)CN5CC(=O)N5CC(C)(C)c6[nH]c(=O)c(Cc7ccc(F)cc7)cc65)[C@H](C)C4)nn3)C[C@H]2C)CN1]

LCMS (ES, m/z): 607.10 [M/2+H]⁺

1H NMR (400 MHz, Methanol-d₄) δ 8.14 (d, J=1.0 Hz, 1H), 7.92 (d, J=7.4 Hz, 1H), 7.88 (s, 1H), 7.24 (ddd, J=8.9, 5.4, 2.0 Hz, 4H), 7.05-6.94 (m, 4H), 4.51 (t, J=7.0 Hz, 2H), 4.04-3.61 (m, 16H), 3.61-3.31 (m, 10H), 3.28-2.83 (m, 18H), 2.66-2.50 (m, 2H), 2.31-2.22 (m, 2H), 1.41 (t, J=6.9 Hz, 13H), 1.37-1.23 (m, 10H), 1.17 (d, J=6.0 Hz, 3H).

### Example 158, 6-(4-fluorobenzyl)-1-(2-((2R,5R)-2-(((R)-4-(2-(1-(5-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-5-oxopentyl)-1H-1,2,3-triazol-4-yl)ethyl)-2-methylpiperazin-1-yl)methyl)-5-methylpiperazin-1-yl)acetyl)-3,3-dimethyl-1,2,3,4-tetrahydro-5H-pyrrolo[3,2-b]pyridin-5-one

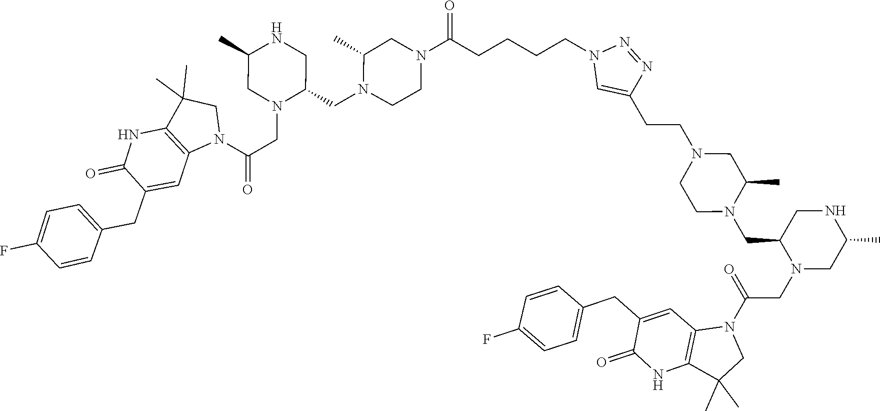 [SMILES: C[C@@H]1CN(CC(=O)N2CC(C)(C)c3[nH]c(=O)c(Cc4ccc(F)cc4)cc32)[C@@H](CN2CCN(CCc3cn(CCCCC(=O)N4CCN(C[C@H]5CN[C@H](C)CN5CC(=O)N5CC(C)(C)c6[nH]c(=O)c(Cc7ccc(F)cc7)cc65)[C@H](C)C4)nn3)C[C@H]2C)CN1]

LCMS (ES, m/z): 614.10 [M/2+H]⁺

1H NMR (400 MHz, Methanol-d₄) δ 8.14 (d, J=0.9 Hz, 1H), 7.91 (d, J=9.2 Hz, 1H), 7.86 (s, 1H), 7.35-7.14 (m, 4H), 7.00 (td, J=8.8, 6.0 Hz, 4H), 4.47 (t, J=6.9 Hz, 2H), 4.04-3.76 (m, 13H), 3.78-3.61 (m, 3H), 3.60-3.31 (m, 10H), 3.29-2.84 (m, 18H), 2.58 (s, 2H), 2.02 (p, J=7.1 Hz, 2H), 1.68 (dt, J=14.4, 6.9 Hz, 2H), 1.41 (t, J=6.3 Hz, 13H), 1.32 (d, J=6.3 Hz, 4H), 1.26 (dd, J=18.5, 6.5 Hz, 6H), 1.18 (d, J=6.1 Hz, 3H).

### Example 159, (S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-(2-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-2-oxoethyl)morpholine-2-carboxamide

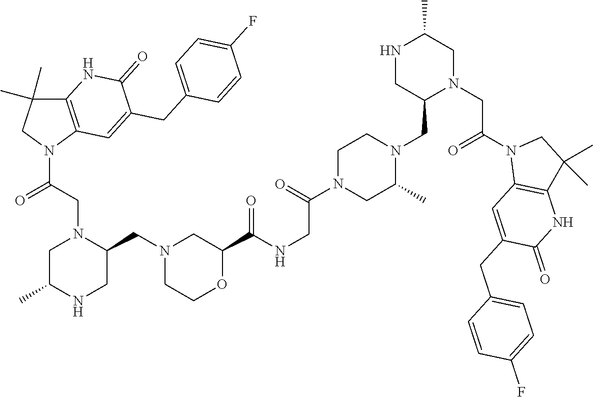

LCMS (ES, m/z): 560.47 [M/2+H]⁺

1H NMR (400 MHz, Methanol-d₄) δ 8.08 (s, 1H), 7.96 (s, 1H), 7.24 (dt, J=9.2, 5.1 Hz, 4H), 7.03 (td, J=8.7, 5.9 Hz, 4H), 4.24-4.09 (m, 3H), 4.07-3.74 (m, 14H), 3.71-3.58 (m, 3H), 3.55-3.46 (m, 2H), 3.41 (s, 2H), 3.29-2.86 (m, 14H), 2.77 (s, 1H), 2.61 (s, 2H), 1.44-1.39 (m, 12H), 1.30 (tt, J=15.9, 8.9 Hz, 12H).

### Example 160, (S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-(4-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-4-oxobutyl)morpholine-2-carboxamide

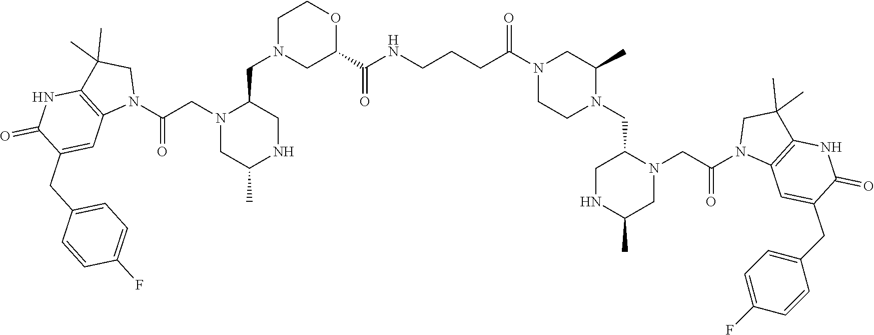 [SMILES: C[C@@H]1CN(CC(=O)N2CC(C)(C)c3[nH]c(=O)c(Cc4ccc(F)cc4)cc32)[C@@H](CN2CCO[C@H](C(=O)NCCCC(=O)N3CCN(C[C@H]4CN[C@H](C)CN4CC(=O)N4CC(C)(C)c5[nH]c(=O)c(Cc6ccc(F)cc6)cc54)[C@H](C)C3)C2)CN1]

LCMS (ES, m/z): 574.40 [M/2+H]⁺

1H NMR (400 MHz, Methanol-d₄) δ 8.07 (s, 1H), 7.92 (d, J=6.5 Hz, 1H), 7.23 (t, J=6.9 Hz, 4H), 7.01 (td, J=8.7, 2.1 Hz, 4H), 4.17-4.05 (m, 1H), 4.05-3.93 (m, 3H), 3.94-3.74 (m, 12H), 3.74-3.36 (m, 10H), 3.29-2.74 (m, 14H), 2.65 (s, 1H), 2.53 (s, 2H), 1.96-1.85 (m, 2H), 1.47-1.38 (m, 12H), 1.39-1.19 (m, 12H).

### Example 161, (R,R,2S,2'S)-N,N′-(1,4-phenylenebis(methylene))bis(4-(((2R,5R)-1-(2-(6-(4-chlorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 622.4 [M/2+H]⁺

¹H NMR (400 MHz, METHANOL-d₄) δ=8.52 (br d, J=6.1 Hz, 1H), 8.01 (s, 1H), 7.27 (s, 4H), 7.21-7.16 (m, 4H), 7.14-7.10 (m, 4H), 4.50-4.41 (m, 2H), 4.34-4.25 (m, 2H), 4.15 (br d, J=11.3 Hz, 2H), 4.03-3.91 (m, 4H), 3.85 (br d, J=10.8 Hz, 2H), 3.81-3.69 (m, 10H), 3.66-3.61 (m, 2H), 3.50-3.34 (m, 6H), 3.28-3.22 (m, 2H), 3.20-3.01 (m, 8H), 2.97-2.88 (m, 2H), 2.75 (br s, 4H), 1.40 (d, J=6.3 Hz, 12H), 1.29 (d, J=6.5 Hz, 6H)

### Example 162, (R,R,2S,2'S)-N,N′-(1,4-phenylenebis(methylene))bis(4-(((2R,5R)-1-(2-(6-(4-bromobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 667.4 [M/2+H]⁺

¹H NMR (400 MHz, METHANOL-d₄) δ=8.44-8.35 (m, 1H), 8.09 (s, 2H), 7.36 (d, J=8.3 Hz, 4H), 7.21 (s, 4H), 7.10 (d, J=8.3 Hz, 4H), 4.46 (br dd, J=6.6, 14.9 Hz, 2H), 4.25 (br dd, J=5.4, 14.7 Hz, 2H), 3.98 (br s, 2H), 3.94-3.60 (m, 18H), 3.48 (br s, 4H), 3.25 (br d, J=10.4 Hz, 4H), 3.10 (br d, J=7.8 Hz, 6H), 2.89 (br s, 4H), 2.41 (br s, 4H), 1.38 (d, J=10.8 Hz, 12H), 1.28 (d, J=6.5 Hz, 6H)

### Example 163, (R,R,2S,2'S)-N,N′-(1,4-phenylenebis(methylene))bis(4-(((2R,5R)-1-(2-(3,3-dimethyl-6-(4-methylbenzyl)-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 602.4 [M/2+H]⁺

¹H NMR (400 MHz, METHANOL-d₄) δ=8.60 (br s, 1H), 7.94 (br s, 2H), 7.30 (d, J=3.1 Hz, 4H), 7.00 (s, 8H), 4.52-4.43 (m, 2H), 4.35-4.27 (m, 2H), 4.13 (br s, 2H), 3.96-3.81 (m, 6H), 3.80-3.62 (m, 10H), 3.61-3.50 (m, 2H), 3.49-3.33 (m, 6H), 3.25-2.86 (m, 12H), 2.81-2.65 (m, 3H), 2.25 (s, 6H), 1.40 (d, J=8.8 Hz, 12H), 1.28 (d, J=6.5 Hz, 6H)

### Example 164, (R,R,2S,2'S)-N,N′-(1,4-phenylenebis(methylene))bis(4-(((2R,5R)-1-(2-(6-(4-chloro-2-hydroxybenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 639.2 [M/2+H]⁺

¹H NMR (400 MHz, METHANOL-d₄) δ=8.13 (s, 2H), 7.24 (s, 4H), 6.99 (d, J=8.0 Hz, 2H), 6.78 (d, J=2.0 Hz, 2H), 6.66 (dd, J=1.9, 8.1 Hz, 2H), 4.48-4.40 (m, 2H), 4.29 (d, J=14.9 Hz, 2H), 4.10 (br d, J=8.1 Hz, 2H), 3.98 (br d, J=8.9 Hz, 2H), 3.94-3.90 (m, 2H), 3.86-3.67 (m, 14H), 3.65-3.60 (m, 2H), 3.45-3.35 (m, 4H), 3.28-3.21 (m, 4H), 3.18-2.95 (m, 10H), 2.84 (br d, J=19.4 Hz, 2H), 2.62 (br s, 2H), 1.39 (d, J=11.3 Hz, 12H), 1.28 (d, J=6.5 Hz, 6H)

### Example 165, (R,R,2S,2'S)-N,N′-(1,4-phenylenebis(methylene))bis(4-(((2R,5R)-1-(2-(6-(2,4-dichlorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 656.3 [M/2+H]⁺

¹H NMR (400 MHz, METHANOL-d₄) δ=7.96 (s, 2H), 7.40 (d, J=1.9 Hz, 2H), 7.22 (s, 8H), 4.46 (d, J=14.8 Hz, 2H), 4.25 (d, J=14.9 Hz, 2H), 4.04-3.93 (m, 2H), 3.90-3.76 (m, 12H), 3.72-3.59 (m, 4H), 3.50-3.43 (m, 2H), 3.40-3.32 (m, 3H), 3.24 (br dd, J=2.1, 12.4 Hz, 4H), 3.14-3.03 (m, 6H), 2.94-2.82 (m, 4H), 2.63-2.49 (m, 2H), 2.47-2.29 (m, 3H), 1.39 (d, J=9.1 Hz, 12H), 1.27 (d, J=6.5 Hz, 6H)

### Example 166, (R,R,2S,2'S)-N,N′-(1,4-phenylenebis(methylene))bis(4-(((2R,5R)-1-(2-(6-(4-(difluoromethyl)benzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 638.4 [M/2+H]⁺

¹H NMR (400 MHz, METHANOL-d₄) δ=8.57 (br s, 1H), 8.04 (s, 2H), 7.36 (br d, J=7.9 Hz, 4H), 7.29-7.22 (m, 8H), 6.85-6.52 (m, 2H), 4.48-4.39 (m, 2H), 4.35-4.25 (m, 2H), 4.10 (br d, J=11.0 Hz, 2H), 3.94 (br d, J=10.6 Hz, 4H), 3.90-3.70 (m, 12H), 3.59 (br t, J=11.3 Hz, 2H), 3.50-3.32 (m, 8H), 3.28-2.97 (m, 10H), 2.97-2.83 (m, 2H), 2.71 (br s, 2H), 1.40 (d, J=7.2 Hz, 12H), 1.28 (d, J=6.5 Hz, 6H)

### Example 167, (2S)-4-[[(2R, 5R)-1-[2-[3,3-dimethyl-5-oxo-6-[(2,4,6-trifluorophenyl)methyl]-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[3,3-dimethyl-5-oxo-6-[(2,4,6-trifluorophenyl)methyl]-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 642.3 [M/2+H]⁺

¹H NMR (400 MHz, METHANOL-d₄) δ=8.52 (br s, 1H), 7.98 (s, 2H), 7.24 (s, 4H), 6.81 (t, J=8.3 Hz, 4H), 4.49-4.41 (m, 2H), 4.34-4.27 (m, 2H), 4.09 (br d, J=10.4 Hz, 2H), 3.96-3.87 (m, 4H), 3.86-3.78 (m, 8H), 3.77-3.68 (m, 4H), 3.56 (br t, J=11.3 Hz, 2H), 3.46-3.34 (m, 4H), 3.29-3.24 (m, 3H), 3.18-3.02 (m, 6H), 3.00-2.91 (m, 3H), 2.85-2.72 (m, 2H), 2.70-2.46 (m, 4H), 1.39 (d, J=11.8 Hz, 12H), 1.29 (d, J=6.6 Hz, 6H)

### Example 168, (2S)-4-[[(2R, 5R)-1-[2-[6-[(4-chloro-2-methyl-phenyl)methyl]-3,3-dimethyl-5-oxo-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[6-[(4-chloro-2-methyl-phenyl)methyl]-3,3-dimethyl-5-oxo-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 637.2 [M/2+H]⁺

¹H NMR (400 MHz, METHANOL-d₄) δ=7.68 (s, 2H), 7.36 (s, 4H), 7.06 (d, J=2.0 Hz, 2H), 7.03-6.99 (m, 2H), 6.98-6.93 (m, 2H), 4.55-4.48 (m, 2H), 4.36-4.19 (m, 4H), 4.00-3.94 (m, 2H), 3.91-3.82 (m, 4H), 3.80-3.68 (m, 10H), 3.60-3.40 (m, 8H), 3.24-3.13 (m, 6H), 3.11-2.98 (m, 6H), 2.95-2.82 (m, 4H), 2.03 (s, 6H), 1.42 (d, J=5.5 Hz, 12H), 1.27 (d, J=6.6 Hz, 6H)

### Example 169, (2S)-4-[[(2R, 5R)-1-[2-[6-[(4-chloro-2-methoxy-phenyl)methyl]-3,3-dimethyl-5-oxo-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[6-[(4-chloro-2-methoxy-phenyl)methyl]-3,3-dimethyl-5-oxo-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 653.2 [M/2+H]⁺

¹H NMR (400 MHz, METHANOL-d₄) δ=8.27 (br t, J=6.1 Hz, 1H), 7.48 (s, 2H), 7.00 (s, 4H), 6.65 (d, J=7.9 Hz, 2H), 6.53 (d, J=1.7 Hz, 2H), 6.44 (dd, J=1.8, 7.9 Hz, 2H), 4.20-4.09 (m, 2H), 4.04-3.95 (m, 2H), 3.85 (br d, J=12.2 Hz, 2H), 3.66-3.59 (m, 2H), 3.57-3.46 (m, 5H), 3.40 (br d, J=19.7 Hz, 8H), 3.34 (s, 2H), 3.29-3.03 (m, 10H), 3.01-2.67 (m, 18H), 2.48 (br s, 4H), 1.08 (d, J=6.8 Hz, 12H), 0.95 (d, J=6.6 Hz, 6H)

### Example 170, (2S)-4-[[(2R, 5R)-1-[2-[6-[[4-chloro-2-(trifluoromethyl)phenyl]methyl]-3,3-dimethyl-5-oxo-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[6-[[4-chloro-2-(trifluoromethyl)phenyl]methyl]-3,3-dimethyl-5-oxo-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 691.0 [M/2+H]⁺

¹H NMR (400 MHz, METHANOL-d₄) δ=8.52 (br s, 1H), 7.84 (s, 2H), 7.66 (d, J=2.0 Hz, 2H), 7.47 (dd, J=2.0, 8.3 Hz, 2H), 7.31-7.21 (m, 6H), 4.60-4.60 (m, 1H), 4.48-4.41 (m, 2H), 4.36-4.28 (m, 2H), 4.10 (br d, J=11.0 Hz, 2H), 4.03-3.89 (m, 8H), 3.87-3.81 (m, 2H), 3.80-3.62 (m, 6H), 3.56 (br t, J=11.3 Hz, 2H), 3.47-3.33 (m, 4H), 3.25 (br dd, J=2.4, 12.8 Hz, 4H), 3.18-3.11 (m, 2H), 3.09-2.93 (m, 6H), 2.91-2.55 (m, 6H), 1.41 (d, J=6.6 Hz, 12H), 1.28 (d, J=6.6 Hz, 6H)

### Example 171, (2S)-4-[[(2R, 5R)-1-[2-[6-[(4-chloro-2-cyano-phenyl)methyl]-3,3-dimethyl-5-oxo-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[6-[(4-chloro-2-cyano-phenyl)methyl]-3,3-dimethyl-5-oxo-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 648.1 [M/2+H]⁺

¹H NMR (400 MHz, METHANOL-d₄) δ=8.26 (s, 2H), 7.60 (d, J=1.8 Hz, 2H), 7.52-7.43 (m, 4H), 7.23 (s, 4H), 4.49-4.41 (m, 2H), 4.33-4.14 (m, 6H), 4.00-3.93 (m, 6H), 3.91-3.70 (m, 10H), 3.45 (br s, 6H), 3.32 (br s, 2H), 3.31-3.30 (m, 4H), 3.29-2.98 (m, 10H), 2.95-2.67 (m, 4H), 1.40 (d, J=8.3 Hz, 12H), 1.29 (d, J=6.4 Hz, 6H)

### Example 172, (2S)-4-[[(2R,5R)-1-[2-[6-[[4-chloro-2-(dimethylcarbamoyl)phenyl]methyl]-5-hydroxy-3,3-dimethyl-2H-pyrrolo[3,2-b]pyridin-1-yl]-2-oxo-ethyl]-5-methyl-piperazin-2-yl]methyl]-N-[[4-[[[(2S)-4-[[(2R,5R)-1-[2-[6-[[4-chloro-2-(dimethylcarbamoyl)phenyl]methyl]-5-hydroxy-3,3-dimethyl-2H-pyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 693.4 [M/2+H]⁺

¹H NMR (400 MHz, METHANOL-d₄) δ=8.14 (s, 2H), 7.42-7.22 (m, 8H), 7.20-7.06 (m, 2H), 4.50-4.26 (m, 6H), 4.20 (br d, J=11.8 Hz, 2H), 4.01-3.95 (m, 2H), 3.84 (br s, 12H), 3.56-3.43 (m, 6H), 3.30-3.14 (m, 8H), 3.12-3.02 (m, 6H), 3.00-2.82 (m, 10H), 2.69 (s, 6H), 1.40 (d, J=5.3 Hz, 12H), 1.30 (d, J=6.5 Hz, 6H)

### Example 173, (2S)-4-[[(2R, 5R)-1-[2-[6-[(2-isopropylphenyl)methyl]-3,3-dimethyl-5-oxo-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[6-[(2-isopropylphenyl)methyl]-3,3-dimethyl-5-oxo-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 630.5 [M/2+H]⁺

¹H NMR (400 MHz, METHANOL-d₄) δ=8.67 (br s, 1H), 7.73 (s, 2H), 7.37 (s, 4H), 7.22-7.12 (m, 4H), 7.06-6.93 (m, 4H), 4.57-4.46 (m, 2H), 4.40-4.32 (m, 2H), 4.09 (br d, J=10.6 Hz, 2H), 3.97 (br d, J=10.6 Hz, 2H), 3.88-3.79 (m, 6H), 3.75-3.63 (m, 8H), 3.50-3.34 (m, 8H), 3.25-3.12 (m, 6H), 3.06-2.93 (m, 6H), 2.85 (td, J=6.8, 13.6 Hz, 3H), 2.78 (br s, 3H), 1.42 (d, J=9.0 Hz, 12H), 1.28 (d, J=6.5 Hz, 6H), 1.10 (d, J=6.8 Hz, 6H), 1.07 (d, J=6.8 Hz, 6H)

### Example 174, (2S)-4-[[(2R,5R)-1-[2-[3,3-dimethyl-5-oxo-6-[(2,3,4,5-tetrafluorophenyl)methyl]-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[3,3-dimethyl-5-oxo-6-[(2,3,4,5-tetrafluorophenyl)methyl]-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 660.4 [M/2+H]⁺.

¹H NMR (400 MHz, METHANOL-d₄) δ=8.44-8.33 (m, 1H), 8.26 (s, 2H), 7.20-7.04 (m, 6H), 4.47-4.38 (m, 2H), 4.30-4.19 (m, 2H), 4.13-3.96 (m, 4H), 3.94-3.80 (m, 10H), 3.79-3.50 (m, 6H), 3.49-3.33 (m, 4H), 3.28-3.23 (m, 2H), 3.22-3.06 (m, 6H), 3.03-2.85 (m, 4H), 2.82-2.34 (m, 6H), 1.39 (d, J=11.8 Hz, 12H), 1.30 (d, J=6.5 Hz, 6H)

### Example 175, (2S)-4-[[(2R,5R)-1-[2-[3,3-dimethyl-5-oxo-6-[(2,4,5-trifluorophenyl)methyl]-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[3,3-dimethyl-5-oxo-6-[(2,4,5-trifluorophenyl)methyl]-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 642.5 [M/2+H]⁺

¹H NMR (400 MHz, METHANOL-d₄) δ=8.18 (s, 2H), 7.27-7.18 (m, 6H), 7.01 (dt, J=6.8, 9.9 Hz, 2H), 4.45-4.27 (m, 4H), 4.22-4.06 (m, 4H), 3.98-3.92 (m, 1H), 3.98-3.92 (m, 1H), 3.89-3.80 (m, 6H), 3.79-3.67 (m, 8H), 3.51-3.35 (m, 6H), 3.30-3.28 (m, 1H), 3.26 (br s, 1H), 3.22-3.01 (m, 8H), 2.98-2.66 (m, 6H), 1.40 (d, J=7.8 Hz, 12H), 1.30 (d, J=6.5 Hz, 6H)

### Example 176, (2S)-4-[[(2R,5R)-1-[2-[3,3-dimethyl-5-oxo-6-[(2,3,4,5,6-pentafluorophenyl)methyl]-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[3,3-dimethyl-5-oxo-6-[(2,3,4,5,6-pentafluorophenyl)methyl]-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 678.3 [M/2+H]⁺

¹H NMR (400 MHz, METHANOL-d₄) δ=8.52 (br t, J=5.8 Hz, 1H), 8.20 (s, 2H), 7.19 (s, 4H), 4.45-4.26 (m, 4H), 4.22-4.02 (m, 4H), 3.98-3.70 (m, 16H), 3.52-3.33 (m, 6H), 3.27 (br s, 2H), 3.23-3.00 (m, 8H), 2.98-2.62 (m, 6H), 1.40 (d, J=11.5 Hz, 12H), 1.30 (br d, J=6.4 Hz, 6H)

### Example 177, (2S)-4-[[(2R,5R)-1-[2-[6-[(2,4-difluorophenyl)methyl]-3,3-dimethyl-5-oxo-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[6-[(2,4-difluorophenyl)methyl]-3,3-dimethyl-5-oxo-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 624.4 [M/2+H]⁺

¹H NMR (400 MHz, METHANOL-d₄) δ=8.56 (br s, 1H), 8.05 (s, 2H), 7.27-7.18 (m, 6H), 6.88-6.77 (m, 4H), 4.49-4.41 (m, 2H), 4.34-4.26 (m, 2H), 4.13 (br d, J=12.3 Hz, 2H), 4.00-3.92 (m, 4H), 3.88-3.65 (m, 14H), 3.64-3.58 (m, 2H), 3.49-3.33 (m, 6H), 3.26 (br dd, J=2.3, 12.7 Hz, 2H), 3.19-3.14 (m, 2H), 3.11-2.96 (m, 6H), 2.93-2.83 (m, 2H), 2.69 (br d, J=8.4 Hz, 2H), 1.40 (d, J=8.9 Hz, 12H), 1.29 (d, J=6.6 Hz, 6H)

### Example 178, (2S)-4-[[(2R,5R)-1-[2-[3,3-dimethyl-5-oxo-6-[(2,3,4-trifluorophenyl)methyl]-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[3,3-dimethyl-5-oxo-6-[(2,3,4-trifluorophenyl)methyl]-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 642.3 [M/2+H]⁺

¹H NMR (400 MHz, METHANOL-d₄) δ=8.53 (br d, J=5.4 Hz, 1H), 8.13 (s, 2H), 7.30-7.12 (m, 4H), 7.06-6.89 (m, 4H), 4.48-4.37 (m, 2H), 4.35-4.24 (m, 2H), 4.19-3.91 (m, 6H), 3.89-3.63 (m, 14H), 3.49-3.37 (m, 4H), 3.29-3.23 (m, 2H), 3.21-2.98 (m, 8H), 2.96-2.58 (m, 6H), 1.40 (br d, J=8.1 Hz, 12H), 1.29 (d, J=6.5 Hz, 6H)

### Example 179, (2S)-4-[[(2R,5R)-1-[2-[6-[(2-cyano-4-fluoro-phenyl)methyl]-3,3-dimethyl-5-oxo-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[6-[(2-cyano-4-fluoro-phenyl)methyl]-3,3-dimethyl-5-oxo-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 631.4 [M/2+H]⁺

¹H NMR (400 MHz, METHANOL-d₄) δ=8.26 (s, 2H), 7.51 (dd, J=5.5, 8.6 Hz, 2H), 7.36 (br d, J=7.8 Hz, 2H), 7.32-7.21 (m, 6H), 4.47-4.38 (m, 2H), 4.32-4.11 (m, 6H), 4.00-3.93 (m, 6H), 3.90-3.68 (m, 10H), 3.54-3.36 (m, 6H), 3.25 (br s, 2H), 3.20-3.00 (m, 8H), 2.97-2.88 (m, 2H), 2.88-2.65 (m, 4H), 1.40 (d, J=7.7 Hz, 12H), 1.29 (d, J=6.4 Hz, 6H)

### Example 180, (R,R,2S,2'S)-N,N′-(1,4-phenylenebis(methylene))bis(4-(((2R,5R)-1-(2-(3,3-dimethyl-5-oxo-6-(2,4,6-trichlorobenzyl)-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 691.3 [M/2+H]⁺

¹H NMR (400 MHz, METHANOL-d₄) δ=7.58 (s, 2H), 7.42-7.30 (m, 8H), 4.58 (br d, J=14.8 Hz, 2H), 4.29-4.15 (m, 4H), 4.11-3.99 (m, 4H), 3.98-3.77 (m, 8H), 3.75-3.63 (m, 6H), 3.55-3.34 (m, 8H), 3.23 (br d, J=12.1 Hz, 2H), 3.18-3.08 (m, 4H), 3.01 (br dd, J=10.5, 14.1 Hz, 6H), 2.87-2.65 (m, 2H), 1.42 (d, J=7.0 Hz, 12H), 1.27 (d, J=6.4 Hz, 6H)

### Example 181, (R,R,2S,2'S)-N,N′-(1,4-phenylenebis(methylene))bis(4-(((2R,5R)-1-(2-(6-(4-fluorophenoxy)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 608.4 [M/2+H]⁺

¹H NMR (400 MHz, METHANOL-d₄) δ=8.57-8.39 (m, 1H), 7.87 (s, 1H), 7.29-7.22 (m, 4H), 7.01 (d, J=6.3 Hz, 8H), 4.44-4.30 (m, 4H), 4.05 (br d, J=10.0 Hz, 2H), 3.98-3.84 (m, 7H), 3.82-3.73 (m, 4H), 3.71-3.62 (m, 2H), 3.56 (br t, J=12.3 Hz, 2H), 3.45-3.34 (m, 4H), 3.26 (br s, 2H), 3.16 (br d, J=14.1 Hz, 3H), 3.10-3.01 (m, 4H), 3.00-2.91 (m, 2H), 2.87-2.75 (m, 2H), 2.62 (ddd, J=7.9, 10.2, 14.9 Hz, 4H), 1.40 (br d, J=7.0 Hz, 12H), 1.29 (br d, J=6.5 Hz, 6H)

### Example 182, (R,R,2S,2'S)-N,N′-(1,4-phenylenebis(methylene))bis(4-(((2R,5R)-1-(2-(6-(4-chlorophenoxy)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 624.4 [M/2+H]⁺

¹H NMR (400 MHz, METHANOL-d₄) δ=7.99 (s, 2H), 7.34-7.19 (m, 8H), 7.01-6.92 (m, 4H), 4.47-4.25 (m, 4H), 4.06-3.34 (m, 20H), 3.29-2.85 (m, 14H), 2.72-2.36 (m, 6H), 1.40 (d, J=8.0 Hz, 12H), 1.30-1.26 (m, 1H), 1.28 (d, J=6.5 Hz, 5H)

### Example 183, (R,R,2S,2'S)-N,N′-(1,4-phenylenebis(methylene))bis(4-(((2R,5R)-1-(2-(6-(4-bromophenoxy)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 669.3 [M/2+H]⁺

¹H NMR (400 MHz, METHANOL-d₄) δ=8.54 (s, 1H), 8.08 (s, 2H), 7.46 (d, J=9.0 Hz, 4H), 7.17 (s, 4H), 6.93 (d, J=9.0 Hz, 4H), 4.44-4.36 (m, 2H), 4.30-4.23 (m, 2H), 3.94-3.82 (m, 6H), 3.80-3.65 (m, 6H), 3.43-3.33 (m, 4H), 3.15 (br s, 4H), 3.06 (br d, J=10.1 Hz, 2H), 2.95 (br d, J=6.6 Hz, 4H), 2.81 (br d, J=12.7 Hz, 2H), 2.72-2.59 (m, 4H), 2.17 (br d, J=14.0 Hz, 2H), 2.06-1.92 (m, 4H), 1.37 (d, J=5.7 Hz, 12H), 1.20 (br d, J=5.7 Hz, 6H)

### Example 184, (R,R,2S,2'S)-N,N′-(1,4-phenylenebis(methylene))bis(4-(((2R,5R)-1-(2-(3,3-dimethyl-5-oxo-6-(p-tolyloxy)-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 604.5 [M/2+H]⁺

¹H NMR (400 MHz, METHANOL-d₄) δ=8.55-8.40 (m, 1H), 7.86-7.80 (m, 1H), 7.30-7.23 (m, 4H), 7.14-7.05 (m, 4H), 6.90 (d, J=7.8 Hz, 4H), 4.47-4.39 (m, 2H), 4.36-4.26 (m, 2H), 4.04-3.88 (m, 5H), 3.87-3.67 (m, 9H), 3.65-3.56 (m, 2H), 3.49-3.42 (m, 2H), 3.39-3.33 (m, 2H), 3.27-3.22 (m, 2H), 3.15-2.99 (m, 6H), 2.96-2.78 (m, 4H), 2.72-2.34 (m, 6H), 2.27 (d, J=3.6 Hz, 6H), 1.38 (br d, J=11.8 Hz, 12H), 1.27 (br d, J=6.5 Hz, 6H)

### Example 185, (R,R,2S,2'S)-N,N′-(1,4-phenylenebis(methylene))bis(4-(((2R,5R)-1-(2-(3,3-dimethyl-5-oxo-6-phenoxy-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 590.5 [M/2+H]⁺

¹H NMR (400 MHz, METHANOL-d₄) δ=8.56 (br s, 1H), 7.83 (s, 2H), 7.34-7.22 (m, 8H), 7.11-6.96 (m, 6H), 4.47-4.30 (m, 4H), 4.08-3.94 (m, 4H), 3.92-3.85 (m, 2H), 3.79 (br s, 5H), 3.71-3.60 (m, 2H), 3.56-3.34 (m, 7H), 3.29-2.94 (m, 12H), 2.91-2.54 (m, 6H), 1.41 (d, J=8.4 Hz, 12H), 1.30 (d, J=6.5 Hz, 6H)

### Example 186, (R,R,2S,2'S)-N,N′-(1,4-phenylenebis(methylene))bis(4-(((2R,5R)-1-(2-(6-benzyl-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide)

LCMS (ES, m/z): 588.5 [M/2+H]⁺

¹H NMR (400 MHz, METHANOL-d₄) δ=8.54 (br dd, J=6.5, 8.4 Hz, 1H), 7.98 (br s, 2H), 7.28 (br s, 4H), 7.23-7.10 (m, 10H), 4.52-4.42 (m, 2H), 4.30 (br d, J=14.1 Hz, 2H), 4.13-3.99 (m, 2H), 3.94-3.81 (m, 6H), 3.80-3.62 (m, 10H), 3.60-3.35 (m, 6H), 3.24 (br d, J=10.8 Hz, 4H), 3.16-2.94 (m, 8H), 2.89-2.71 (m, 2H), 2.70-2.49 (m, 4H), 1.40 (d, J=9.4 Hz, 12H), 1.28 (d, J=6.5 Hz, 6H)

### Example 187, 1,1′-(2,2′-((3R,3′R,6R,6′R)-6,6′-(((2S,2'S)-2,2′-((1,3-phenylenebis(oxy))bis(methylene))bis(morpholine-4,2-diyl))bis(methylene))bis(3-methylpiperazine-6,1-diyl))bis(acetyl))bis(6-(2,4-dichlorobenzyl)-3,3-dimethyl-2,3-dihydro-1H-pyrrolo[3,2-b]pyridin-5(4H)-one)

LCMS (ES, m/z): 630.3 [M/2+H]⁺

¹H NMR (400 MHz, METHANOL-d₄) δ=7.98 (s, 2H), 7.43 (s, 2H), 7.25 (s, 4H), 7.19 (t, J=8.3 Hz, 1H), 6.54 (br d, J=8.3 Hz, 2H), 6.39 (br s, 1H), 4.09-3.57 (m, 28H), 3.51-3.37 (m, 4H), 3.27-3.19 (m, 4H), 3.18-2.93 (m, 10H), 1.42 (br d, J=15.6 Hz, 12H), 1.29 (br s, 6H)

### Example 188, 1-[2-[(2R,5R)-2-[[(2S)-2-[[3-[[(2S)-4-[[(2R,5R)-1-[2-[3,3-dimethyl-5-oxo-6-[(2,4,6-trifluorophenyl)methyl]-2,4-dihydropyrrolo[3,2-b]pyridin-1-yl]-2-oxo-ethyl]-5-methyl-piperazin-2-yl]methyl]morpholin-2-yl]methoxy]phenoxy]methyl]morpholin-4-yl]methyl]-5-methyl-piperazin-1-yl]acetyl]-3,3-dimethyl-6-[(2,4,6-trifluorophenyl)methyl]-2,4-dihydropyrrolo[3,2-b]pyridin-5-one

LCMS (ES, m/z): 615.5 [M/2+H]⁺

¹H NMR (400 MHz, METHANOL-d₄) δ=8.03 (s, 2H), 7.18 (t, J=8.3 Hz, 1H), 6.84 (br t, J=8.3 Hz, 4H), 6.54 (dd, J=2.1, 8.3 Hz, 2H), 6.39 (br s, 1H), 4.15-3.64 (m, 26H), 3.47 (br s, 4H), 3.30-3.14 (m, 8H), 3.06 (br dd, J=10.6, 13.8 Hz, 10H), 1.43 (d, J=13.8 Hz, 12H), 1.30 (d, J=6.5 Hz, 6H)

### Example 189, 5-chloro-2-[[1-[2-[(2R,5R)-2-[[(2S)-2-[[3-[[(2S)-4-[[(2R,5R)-1-[2-[6-[(4-chloro-2-cyano-phenyl)methyl]-3,3-dimethyl-5-oxo-2,4-dihydropyrrolo[3,2-b]pyridin-1-yl]-2-oxo-ethyl]-5-methyl-piperazin-2-yl]methyl]morpholin-2-yl]methoxy]phenoxy]methyl]morpholin-4-yl]methyl]-5-methyl-piperazin-1-yl]acetyl]-3,3-dimethyl-5-oxo-2,4-dihydropyrrolo[3,2-b]pyridin-6-yl]methyl]benzonitrile

LCMS (ES, m/z): 621.3 [M/2+H]⁺

¹H NMR (400 MHz, METHANOL-d₄) δ=8.28 (s, 2H), 7.64 (s, 2H), 7.56-7.44 (m, 4H), 7.14 (t, J=8.3 Hz, 1H), 6.51 (dd, J=2.2, 8.2 Hz, 2H), 6.39 (br s, 1H), 4.23-4.03 (m, 6H), 4.03-3.92 (m, 8H), 3.89 (br d, J=10.4 Hz, 2H), 3.87-3.71 (m, 8H), 3.57-3.42 (m, 4H), 3.41-3.34 (m, 2H), 3.29-3.18 (m, 6H), 3.17-2.83 (m, 10H), 1.41 (d, J=11.9 Hz, 12H), 1.30 (d, J=6.5 Hz, 6H)

### Example 190, (2S)-4-[[(2R,5R)-1-[2-[3,3-dimethyl-5-oxo-6-[(2,4,5-trifluorophenyl)methyl]-2,4-dihydropyrrolo[3,2-b]pyridin-1-yl]-2-oxo-ethyl]-5-methyl-piperazin-2-yl]methyl]-N-[[4-[[[(2S)-4-[[(2R,5R)-1-[2-[3,3-dimethyl-5-oxo-6-[(2,4,5-trifluorophenyl)methyl]-2,4-dihydropyrrolo[3,2-b]pyridin-1-yl]-2-oxo-ethyl]-5-methyl-piperazin-2-yl]methyl]morpholine-2-carbonyl]amino]methyl]phenyl]methyl]morpholine-2-carboxamide

LCMS (ES, m/z): 597.4 [M/2+H]⁺

¹H NMR (400 MHz, METHANOL-d₄) δ=8.06 (br s, 2H), 7.30-7.22 (m, 2H), 7.18 (t, J=8.3 Hz, 1H), 6.92-6.80 (m, 4H), 6.55 (br d, J=8.1 Hz, 2H), 6.43 (br s, 1H), 4.19-3.95 (m, 10H), 3.88 (br d, J=11.4 Hz, 2H), 3.82-3.70 (m, 12H), 3.58-3.40 (m, 6H), 3.30-3.16 (m, 8H), 3.07 (br d, J=10.4 Hz, 8H), 1.42 (br d, J=13.0 Hz, 12H), 1.30 (br d, J=6.4 Hz, 6H)

### Jurkat Latency Reversal Assay

Jurkat HIV-luciferase clones were maintained in RPMI medium 1640 (Gibco by Life Technologies) containing 10% (vol/vol) fetal bovine serum (SAFC/Sigma-Aldrich) and 25 units/mL penicillin, 25 units/mL streptomycin (Gibco by Life Technologies), and were split 1:4 every 3 to 4 days to maintain a cell density of ~0.3 to 1 million cells/mL. The Jurkat clones were maintained with the addition of 500 nM EFV in the medium. Three Jurkat cell clones (C16, 115, and N6), each harboring one or two integrated HIV proviruses expressing the luciferase reporter gene, were added at equal amounts for a total of 5,000 cells per well to 384-well plates containing compound titrations. Dose-response testing was performed on compounds dissolved in dimethyl sulfoxide (DMSO; Fisher Scientific, Merelbeke, Belgium) dispensed in duplicate serial 3-fold, 14-point titrations using a D300e Digital Droplet Dispenser (Hewlett-Packard) to give final assay concentrations of 10 μM to 2.1 μM in 50 μL of medium at 0.5% DMSO (vol/vol) final concentration. Cells and compound were incubated at 37° C. for 48 hours, unless otherwise indicated, followed by the addition of 20 μL of Steady-Glo® Luciferase (Promega). Luminescence resulting from the induction of the virally expressed luciferase was measured using an EnVision 2102 Multilabel Plate Reader (Perkin Elmer). Dose-response relationships were analyzed with GraphPad PRISM 6 using a four-parameter logistic regression model to calculate the concentration of compound that gives half-maximal response (EC₅₀) and the maximal percent activation compared to the vehicle control.

### Jurkat Cytotoxicity Assay

Cell viability of Jurkat cells was determined using the CellTiter-Glo® Luminescent Cell Viability Assay (Promega), a homogeneous method to determine the ATP levels in a culture well, which corresponds to the presence of metabolically active cells in culture. Cells were cultured as indicated elsewhere for the Jurkat assays. A proportion of cells was removed and 30 μL of Promega CellTiter-Glo® reagent was added to each well containing cells and luminescence was measured using a Perkin Elmer EnVision plate reader. Dose-response relationships were analyzed with GraphPad PRISM 6 using a four-parameter model logistic regression model to calculate the concentration of compound that reduces cell viability by 50% when compared to untreated controls (CC50).

### BIR Binding Assay

The BIR domain binding assays are FRET based competition assays that utilize His-tagged versions of each of the BIR2 and BIR3 domains from cIAP1, cIAP2, and XIAP (each domain assayed separately) each at a custom optimal concentration and a probe, 200 nM SMAC/DIABLO peptide AVPIAQKSE labelled with AlexaFluor647 (part #crb 1110326h, Discovery Peptides). The assay is conducted in 50 mM HEPES, 150 nM NaCl, 1 mM CHAPS, 5% Glycerol, 1 mM DTT, in dI water with a final pH of 7.4 and final volume of 20 uL. Final protein domain concentrations are: 50 nM cIAP1-BIR3 (Part #APT-11-370 Reaction Biology), 100 nM XIAP-BIR3 (Part #APT-11-351 Reaction Biology), 200 nM XIAP-BIR2 (Part #APT-11-470 Reaction Biology), 325 nM cIAP2-BIR2 (Part #APT-11-489 Reaction Biology), 50 nM cIAP2-BIR3 (Part #APT-11-372 Reaction Biology), 325 nM cIAP1-BIR2 (Part #APT-11-487 Reaction Biology). Compounds were plated using a HP Tecan D300 printer in a final total volume of 202 nl and DMSO concentration normalized across the 15-point, 3-fold dilution series. Peptide and probe were prepared at 2× relative to the concentrations above in binding buffer, 10 uL added to the prepared compound plates, and incubated for 60 minutes. LANCE Eu-W1024 (part #AD0401 Perkin Elmer #, vendor) was prepared at 2× concentration in binding buffer for final concentration of 2 nM, 10 uL added to each well, and plates incubated for 30 minutes. Plates were read on an ENVISION multifunction plate reader with 320 nM laser excitation to obtain the 615 nm/665 nm emission ratio as indicative of probe:protein proximity. Data was analyzed using GraphPad PRISM and curves fit using log(inhibitor) vs. response—Variable slope (four parametersXXX method to obtain IC50 and pIC50 values.

The assay results are set forth in Table 8, with desired properties denoted with “+” symbols and non-desired properties denoted with “−” symbols as defined below. Compounds were optimized to have potency in the Jurkat latency reversal assay, limited or no cytotoxicity, and increased fold separation between BIR3 and BIR2 binding activity, specifically with respect to cIAP1 and cIAP2 BIR3.
^{a} Jurkat assay potency. ++++: 9.31>pEC₅₀>=7.90; +++: 7.90>pEC₅₀>=7.20; ++: 7.20>pEC₅₀>=6.50; +: 6.50>pEC₅₀>=5.48.
^{b} Jurkat assay toxicity. ++++: pCC₅₀<5.00; +++: 5.00<=pCC50<5.50; ++: 5.50<=pCC₅₀<6.00; +: 6.00<=pCC₅₀<6.72.
^{c} IAP1 BIR3 binding assays. ++++: 8.86>pIC₅₀>=8.10; +++: 8.10>pIC₅₀>=7.80; ++: 7.80>pIC₅₀>=7.50; +: 7.50>pIC₅₀>=6.30.
^{d} cIAP1 BIR2 binding assays. +: 4.29<pIC₅₀<=5.00; ++: 5.00<pIC₅₀<=5.50; +++: 5.50<pIC₅₀<=6.00; ++++: 6.00<pIC₅₀<=7.65.
^{e} cIAP1 BIR3 versus BIR2 binding assay window (fold separation (FS) for cIAP1). ++++: FS>10,000; +++: 10,000>FS>1,000; ++: 1,000>FS>100; +: 100>FS>1.
^{f} cIAP2 BIR3 binding assays. ++++: 8.86>pIC₅₀>=8.10; +++: 8.10>pIC₅₀>=7.80; ++: 7.80>pIC₅₀>=7.50; +: 7.50>pIC₅₀>=6.30.
^{g} cIAP2 BIR2 binding assays. +: 4.29<pIC₅₀<=5.00; ++: 5.00<pIC₅₀<=5.50; +++: 5.50<pIC₅₀<=6.00; ++++: 6.00<pIC₅₀<=7.65.
^{h} cIAP2 BIR3 versus BIR2 binding assay window (fold separation (FS) for cIAP2). ++++: FS>10,000; +++: 10,000>FS>1,000; ++: 1,000>FS>100; +: 100>FS>1.
ⁱ XIAP BIR3 binding assays. ++++: 8.86>pIC₅₀>=8.10; +++: 8.10>pIC₅₀>=7.80; ++: 7.80>pIC₅₀>=7.50; +: 7.50>pIC₅₀>=6.30.
^{j} XIAP BIR2 binding assays. +: 4.29<pIC₅₀<=5.00; ++: 5.00<pIC₅₀<=5.50; +++: 5.50<pIC₅₀<=6.00; ++++: 6.00<pIC₅₀<=7.65.
^{k} XIAP BIR3 versus BIR2 binding assay window (fold separation (FS) for XIAP). ++++: FS>10,000; +++: 10,000>FS>1,000; ++: 1,000>FS>100; +: 100>FS>1.

TABLE 8

| | | | | | cIAP1 BIR3 | | | cIAP2 BIR3 | | | XIAP BIR3 |
|---|---|---|---|---|---|---|---|---|---|---|---|
| | | | cIAP1 | cIAP1 | vs BIR2 | cIAP2 | cIAP2 | vs BIR2 | XIAP | XIAP | vs BIR2 |
| Compound | Jurkat | Jurkat | BIR3 | BIR2 | separation | BIR3 | BIR2 | separation | BIR3 | BIR2 | separation |
| No. | pEC₅₀^{a} | pCC₅₀^{b} | (pIC50)^{c} | (pIC50)^{d} | window^{e} | (pIC50)^{f} | (pIC50)^{g} | window^{h} | (pIC50)ⁱ | (pIC50)^{j} | window^{k} |
| 1 | ++ | ++++ | ++ | +++ | ++ | ++ | +++ | ++ | ++ | +++ | ++ |
| 2 | +++ | ++++ | + | +++ | ++ | ++ | +++ | ++ | + | +++ | ++ |
| 3 | +++ | ++++ | +++ | ++++ | ++ | ++ | +++ | ++ | + | + | +++ |
| 4 | ++ | +++ | + | ND | ND | ++ | ND | ND | + | + | ++++ |
| 5 | ++ | ++++ | + | ++ | ++ | ++ | +++ | ++ | + | + | ++++ |
| 6 | ++ | ++++ | ++++ | ++++ | +++ | ++++ | ++++ | +++ | ++++ | + | ++++ |
| 7 | ++ | ++++ | ++ | ++++ | ++ | ++ | ++++ | ++ | ++ | +++ | ++ |
| 8 | + | ++++ | ++ | ++++ | ++ | ++ | +++ | ++ | ++ | + | ++++ |
| 9 | ++ | ++++ | ++ | ++++ | ++ | ++ | +++ | ++ | + | + | +++ |
| 10 | ++ | ++++ | ++ | ++++ | ++ | ++ | ++++ | ++ | ++ | +++ | ++ |
| 11 | ++ | ++++ | ++ | +++ | ++ | ++ | +++ | ++ | + | + | ++++ |
| 12 | ++ | ++++ | ++ | ++++ | ++ | ++ | +++ | ++ | ++ | + | ++++ |
| 13 | ++ | ++++ | ++ | ++++ | ++ | ++ | +++ | ++ | ++ | ND | ND |
| 14 | ++ | ++++ | ++ | ++++ | ++ | ++ | ++++ | ++ | ++ | ND | ND |
| 15 | ++ | ++++ | ++++ | ++++ | ++ | ++++ | ++++ | +++ | ++++ | + | ++++ |
| 16 | +++ | ++++ | ++ | +++ | ++ | ++ | +++ | ++ | ++ | + | ++++ |
| 17 | + | ++++ | ++++ | ++++ | ++ | ++++ | ++++ | ++ | ++++ | + | ++++ |
| 18 | +++ | ++++ | ++++ | +++ | +++ | ++++ | +++ | +++ | ++++ | + | ++++ |
| 19 | ++ | ++++ | ++++ | ++++ | +++ | ++++ | ++++ | +++ | ++++ | + | ++++ |
| 20 | +++ | ++++ | ++ | ++++ | + | ++ | ++++ | ++ | + | ND | ND |
| 21 | ++ | ++++ | + | +++ | ++ | + | +++ | ++ | + | + | +++ |
| 22 | ++ | ++++ | +++ | ++++ | ++ | +++ | ++++ | ++ | +++ | + | ++++ |
| 23 | ++ | ++++ | ++++ | +++ | +++ | ++++ | +++ | +++ | ++++ | + | ++++ |
| 24 | +++ | +++ | + | +++ | ++ | + | +++ | ++ | + | + | +++ |
| 25 | +++ | + | +++ | ++++ | ++ | ++++ | ++++ | +++ | +++ | ND | ND |
| 26 | +++ | + | + | +++ | ++ | +++ | +++ | +++ | ++ | ND | ND |
| 27 | ++++ | +++ | ++ | +++ | ++ | +++ | +++ | +++ | + | + | ++++ |
| 28 | + | + | ++ | +++ | +++ | ++ | ++ | +++ | + | + | +++ |
| 29 | ++++ | +++ | ++ | ND | ND | ++ | ND | ND | ++ | ND | ND |
| 30 | + | ++++ | + | ND | ND | ++ | ND | ND | + | ND | ND |
| 31 | ++ | ++ | + | ++ | ++ | + | ++ | ++ | + | +++ | ++ |
| 32 | + | ++++ | + | ++ | ++ | + | ++ | ++ | + | +++ | ++ |
| 33 | + | ++++ | + | ++ | ++ | + | ++ | ++ | + | +++ | ++ |
| 34 | ++ | ++++ | ++ | +++ | ++ | + | ++ | ++ | + | + | +++ |
| 35 | ++ | ++++ | ++ | ++ | +++ | ++ | + | +++ | + | + | +++ |
| 36 | ++++ | +++ | ++ | ++ | +++ | ++ | ND | ND | + | ND | ND |
| 37 | + | ++++ | + | ++ | +++ | + | ++ | ++ | + | + | ++++ |
| 38 | ++ | ++++ | + | ++ | ++ | + | ++ | ++ | + | + | ++++ |
| 39 | ++ | ++++ | ++ | +++ | ++ | ++ | +++ | +++ | + | + | +++ |
| 40 | ++ | +++ | ++ | + | +++ | ++ | + | +++ | + | ND | ND |
| 41 | + | ++++ | + | ++ | +++ | + | ++ | +++ | + | + | +++ |
| 42 | + | ++++ | + | ++ | ++ | ++ | ++ | +++ | + | + | +++ |
| 43 | + | ++++ | ++ | + | +++ | ++ | + | +++ | + | + | ++++ |
| 44 | + | ++++ | + | + | +++ | ++ | + | ++++ | ++ | + | ++++ |
| 45 | ++ | ++++ | +++ | ++ | +++ | +++ | ++ | +++ | + | + | ++++ |
| 46 | ++ | +++ | ++ | ++ | +++ | ++ | ND | ND | + | ND | ND |
| 47 | + | ++++ | ++ | + | +++ | ++ | + | ++++ | ++ | + | ++++ |
| 48 | + | ++++ | ++ | + | +++ | ++ | + | ++++ | ++ | + | ++++ |
| 49 | +++ | ++++ | ++++ | +++ | +++ | ++++ | ++ | ++++ | ++++ | + | ++++ |
| 50 | ++++ | + | ++++ | ND | ND | ++++ | ND | ND | ++++ | ND | ND |
| 51 | ++++ | ++ | ++++ | ND | ND | +++ | ND | ND | ++ | ND | ND |
| 52 | ++++ | ++ | ++++ | ND | ND | +++ | ND | ND | ++ | ND | ND |
| 53 | +++ | ++ | + | ND | ND | + | ND | ND | + | ND | ND |
| 54 | ++++ | ++ | ++ | ND | ND | ++ | ND | ND | ++ | ND | ND |
| 55 | ++++ | ++ | + | + | ++++ | +++ | + | ++++ | ++ | ND | ND |
| 56 | +++ | + | + | ND | ND | + | ND | ND | + | ND | ND |
| 57 | +++ | ++++ | + | ND | ND | ++ | ND | ND | + | ND | ND |
| 58 | +++ | + | ++ | ND | ND | +++ | ND | ND | ++ | ND | ND |
| 59 | ++++ | +++ | ++ | ++ | +++ | +++ | ++ | +++ | ++ | ND | ND |
| 60 | +++ | + | + | ND | ND | ++ | + | ++++ | + | + | ++++ |
| 61 | +++ | + | + | ND | ND | + | ND | ND | + | ND | ND |
| 62 | +++ | + | + | ND | ND | + | ND | ND | + | ND | ND |
| 63 | +++ | ++++ | +++ | +++ | +++ | ++ | ++ | +++ | ++ | + | ++++ |
| 64 | ++++ | +++ | +++ | + | ++++ | ++++ | + | ++++ | +++ | ND | ND |
| 65 | ++++ | +++ | +++ | ND | ND | ++ | ++++ | + | +++ | ND | ND |
| 66 | ++++ | + | ++ | ND | ND | +++ | ND | ND | ++ | + | ++++ |
| 67 | ++++ | ++ | +++ | +++ | +++ | +++ | ++ | +++ | +++ | ND | ND |
| 68 | ++++ | +++ | ++ | ++ | +++ | +++ | +++ | +++ | +++ | + | +++ |
| 69 | ++++ | +++ | +++ | +++ | +++ | +++ | +++ | +++ | ++ | + | ++++ |
| 70 | ++++ | +++ | ++ | +++ | +++ | +++ | +++ | +++ | ++ | + | ++++ |
| 71 | ++ | ++++ | +++ | +++ | ++ | +++ | +++ | +++ | ++ | + | ++++ |
| 72 | ++++ | +++ | +++ | ++++ | ++ | ++ | +++ | +++ | ++ | + | +++ |
| 73 | ++++ | ++ | +++ | ++ | +++ | ++++ | ++ | +++ | ++ | ND | ND |
| 74 | ++++ | +++ | ++++ | +++ | +++ | ++++ | +++ | +++ | ++++ | ND | ND |
| 75 | ++++ | ++++ | ++++ | ++++ | +++ | ++++ | ++++ | +++ | ++++ | + | ++++ |
| 76 | ++++ | +++ | +++ | ++ | +++ | ++++ | +++ | +++ | ++ | ND | ND |
| 77 | +++ | ++++ | ++++ | +++ | +++ | ++++ | +++ | +++ | ++ | + | ++++ |
| 78 | +++ | +++ | ++++ | ND | ND | ++++ | ND | ND | +++ | ++ | +++ |
| 79 | ++++ | ++ | ++++ | +++ | +++ | ++++ | ++ | +++ | +++ | + | ++++ |
| 80 | ++++ | ++++ | ++++ | +++ | +++ | ++++ | ++ | +++ | ++++ | + | ++++ |
| 81 | ++++ | ++++ | ++ | ND | ND | ++ | ND | ND | ++ | ND | ND |
| 82 | ++++ | ++++ | ++ | + | ++++ | ++ | +++ | +++ | + | ND | ND |
| 83 | +++ | ++++ | ++ | ++ | +++ | ++ | ++ | +++ | ++ | ND | ND |
| 84 | ++++ | ++++ | +++ | +++ | +++ | +++ | ++ | +++ | ++ | + | ++++ |
| 85 | ++++ | ++ | +++ | ++ | +++ | ++ | ++ | +++ | + | + | ++++ |
| 86 | ++++ | +++ | +++ | +++ | +++ | +++ | +++ | +++ | ++ | ND | ND |
| 87 | ++++ | +++ | +++ | +++ | +++ | +++ | ++ | +++ | +++ | + | ++++ |
| 88 | ++++ | +++ | +++ | +++ | +++ | +++ | +++ | +++ | +++ | + | ++++ |
| 89 | ++++ | ++++ | ++ | ++ | +++ | +++ | +++ | +++ | ++ | ND | ND |
| 90 | +++ | ++++ | ++++ | +++ | +++ | ++++ | ++++ | +++ | +++ | + | ++++ |
| 91 | ++++ | ++++ | +++ | ++++ | ++ | +++ | +++ | +++ | +++ | + | ++++ |
| 92 | +++ | ++++ | ++++ | +++ | +++ | +++ | +++ | +++ | +++ | + | ++++ |
| 93 | +++ | ++++ | +++ | ++++ | ++ | +++ | +++ | +++ | + | + | ++++ |
| 94 | ++++ | + | +++ | ND | ND | +++ | ND | ND | ++ | ND | ND |
| 95 | ++ | ++++ | + | +++ | ++ | ++++ | +++ | +++ | +++ | + | ++++ |
| 96 | ++ | ++++ | ++++ | +++ | +++ | +++ | +++ | +++ | +++ | + | ++++ |
| 97 | ++ | ++++ | ++++ | +++ | ++++ | ++++ | ++ | +++ | +++ | + | ++++ |
| 98 | ++ | ++++ | ++++ | +++ | +++ | ++++ | + | +++ | +++ | + | ++++ |
| 99 | ++ | ++++ | ++++ | ++ | +++ | +++ | ++ | +++ | +++ | + | ++++ |
| 100 | ++++ | + | +++ | +++ | +++ | +++ | ++ | +++ | ++ | + | ++++ |
| 101 | ++ | ++++ | ++++ | ++ | +++ | ++++ | ++ | ++++ | +++ | + | ++++ |
| 102 | ++ | ++++ | +++ | ++ | +++ | +++ | ++ | ++++ | +++ | + | ++++ |
| 103 | ++ | ++++ | +++ | ++ | +++ | +++ | ++ | +++ | +++ | + | ++++ |
| 104 | ++ | ++++ | ++++ | +++ | +++ | ++++ | +++ | +++ | ++++ | + | ++++ |
| 105 | ++ | ++++ | +++ | ++ | +++ | +++ | ++ | +++ | +++ | + | ++++ |
| 106 | +++ | ++++ | ++++ | +++ | +++ | ++++ | +++ | +++ | ++++ | + | ++++ |
| 107 | +++ | + | +++ | +++ | +++ | ++++ | + | ++++ | +++ | + | ++++ |
| 108 | +++ | + | ++ | ND | ND | ++ | + | ND | + | ND | ND |
| 109 | +++ | + | ++ | ND | ND | +++ | ND | ND | ND | ND | ND |
| 110 | ++++ | + | ++ | ND | ND | ++ | ND | ND | ++ | + | ++++ |
| 111 | +++ | ++++ | +++ | ++ | +++ | +++ | ++ | +++ | + | + | +++ |
| 112 | +++ | ++++ | +++ | ++ | +++ | +++ | +++ | +++ | ++ | + | +++ |
| 113 | ++++ | ++++ | +++ | ++ | +++ | +++ | ++ | +++ | ++ | + | ++++ |
| 114 | + | ++++ | +++ | +++ | +++ | ++ | ++ | +++ | ++ | + | ++++ |
| 115 | ++ | +++ | + | ++ | +++ | + | + | +++ | + | ++ | ++ |
| 116 | + | ++++ | ++ | ++ | +++ | ++ | ++ | +++ | + | ND | ND |
| 117 | ++ | ++++ | ++ | ++ | +++ | ++ | ++ | +++ | + | + | ++++ |
| 118 | +++ | ++++ | +++ | +++ | +++ | +++ | +++ | +++ | +++ | + | ++++ |
| 119 | +++ | ++++ | ++++ | +++ | +++ | ++++ | ++++ | +++ | +++ | ND | ND |
| 120 | ++ | +++ | +++ | +++ | +++ | +++ | +++ | +++ | ++ | ND | ND |
| 121 | ++ | ++++ | +++ | +++ | +++ | +++ | +++ | +++ | +++ | + | ++++ |
| 122 | +++ | ++++ | +++ | +++ | ++ | +++ | +++ | +++ | ++ | + | ++++ |
| 123 | + | ++++ | +++ | ++ | +++ | +++ | ++ | +++ | +++ | + | ++++ |
| 124 | +++ | ++++ | +++ | +++ | +++ | +++ | +++ | +++ | +++ | + | ++++ |
| 125 | ++ | ++++ | +++ | +++ | +++ | +++ | ++ | +++ | ++ | + | ++++ |
| 126 | ++ | ++++ | +++ | +++ | +++ | +++ | +++ | +++ | +++ | + | ++++ |
| 127 | ++ | ++++ | +++ | +++ | +++ | +++ | +++ | +++ | +++ | + | ++++ |
| 128 | + | ++++ | ++ | ++ | +++ | +++ | ++ | +++ | ++ | + | ++++ |
| 129 | ++++ | ++++ | +++ | ++++ | ++ | +++ | +++ | +++ | ++ | + | ++++ |
| 130 | +++ | ++++ | ++++ | +++ | +++ | ++++ | +++ | +++ | +++ | + | ++++ |
| 131 | +++ | ++++ | + | +++ | ++ | +++ | +++ | +++ | ++ | + | ++++ |
| 132 | +++ | ++++ | +++ | ++ | +++ | +++ | +++ | +++ | ++ | + | ++++ |
| 133 | ++ | ++++ | + | +++ | ++ | +++ | ++ | +++ | ++ | + | ++++ |
| 134 | +++ | ++++ | ++ | +++ | ++ | +++ | +++ | +++ | + | + | +++ |
| 135 | ++ | ++++ | ++ | ++ | +++ | +++ | ++ | +++ | ++ | + | ++++ |
| 136 | ++ | ++++ | +++ | +++ | +++ | +++ | +++ | +++ | ++ | + | ++++ |
| 137 | ++ | ++++ | +++ | +++ | +++ | +++ | +++ | +++ | ++ | + | ++++ |
| 138 | ++ | ++++ | ++++ | +++ | +++ | ++++ | +++ | +++ | +++ | + | ++++ |
| 139 | ++ | ++++ | +++ | +++ | +++ | +++ | +++ | +++ | + | + | +++ |
| 140 | ++ | ++++ | +++ | +++ | +++ | +++ | +++ | +++ | ++ | + | ++++ |
| 141 | +++ | ++++ | +++ | +++ | +++ | +++ | +++ | +++ | ++ | + | +++ |
| 142 | +++ | ++ | ++ | + | +++ | ++ | ND | ND | + | + | +++ |
| 143 | + | ++++ | + | + | ND | ++ | + | ++++ | + | + | +++ |
| 144 | + | +++ | + | + | +++ | + | + | +++ | + | + | +++ |
| 145 | +++ | +++ | +++ | ++ | +++ | +++ | + | +++ | + | + | ++++ |
| 146 | + | ++++ | ++++ | + | ++++ | ++++ | + | ++++ | + | + | ++++ |
| 147 | +++ | ++ | ++ | ++ | +++ | ++ | ND | ND | + | ND | ND |
| 148 | +++ | ++ | ++ | + | ++++ | ++ | ND | ND | + | ND | ND |
| 149 | ++ | ++ | + | + | +++ | + | ND | ND | + | ND | ND |
| 150 | +++ | +++ | +++ | ++ | +++ | +++ | + | ++++ | ++ | + | ++++ |
| 151 | ++ | +++ | ++ | ++ | +++ | +++ | ++ | +++ | + | ND | ND |
| 152 | ++ | +++ | +++ | ++ | +++ | +++ | ++ | +++ | + | ND | ND |
| 153 | ++ | +++ | + | + | +++ | + | ND | ND | + | ND | ND |
| 154 | ++ | ++++ | +++ | +++ | +++ | +++ | +++ | +++ | +++ | + | ++++ |
| 155 | + | ++++ | ++++ | +++ | +++ | +++ | +++ | +++ | ++ | + | ++++ |
| 156 | ++ | ++++ | ++ | +++ | ++ | +++ | +++ | +++ | ++ | + | ++++ |
| 157 | ++ | ++++ | +++ | +++ | ++ | +++ | ++++ | ++ | ++ | + | ++++ |
| 158 | ++ | ++++ | +++ | +++ | ++ | +++ | ++++ | ++ | +++ | + | ++++ |
| 159 | ++ | ++++ | +++ | ++++ | ++ | ++++ | ++++ | +++ | +++ | + | ++++ |
| 160 | ++ | ++++ | +++ | +++ | ++ | +++ | +++ | +++ | +++ | + | ++++ |
| 161 | ++++ | ++++ | ++++ | ++++ | +++ | ++++ | + | ++++ | +++ | ND | ND |
| 162 | +++ | ++++ | ++++ | ++++ | ++ | ++++ | ++++ | +++ | ++ | ND | ND |
| 163 | +++ | ++++ | ++++ | ++++ | ++ | ++++ | ++++ | +++ | +++ | + | ++++ |
| 164 | ++ | ++++ | ++++ | ++++ | ++ | ++++ | ++++ | ++ | +++ | ND | ND |
| 165 | +++ | ++++ | +++ | +++ | +++ | ++++ | ++ | +++ | + | ND | ND |
| 166 | + | ++++ | +++ | ++++ | ++ | ++++ | ++++ | +++ | ++ | + | ++++ |
| 167 | ++++ | ++++ | +++ | ++++ | ++ | ++++ | ++++ | ++ | ++ | + | ++++ |
| 168 | ++++ | ++++ | ++ | ND | ND | +++ | ++++ | ++ | + | ND | ND |
| 169 | +++ | ++++ | +++ | ND | ND | ++++ | ND | ND | + | ND | ND |
| 170 | +++ | ++ | ++++ | ++ | +++ | ++++ | +++ | +++ | ++ | ND | ND |
| 171 | +++ | ++++ | ++++ | ++++ | ++ | ++++ | ++++ | ++ | + | ND | ND |
| 172 | + | ++++ | +++ | + | +++ | +++ | + | ++++ | + | + | ++++ |
| 173 | ++ | +++ | + | + | +++ | ++ | ++ | +++ | + | ND | ND |
| 174 | ++++ | +++ | +++ | ++ | +++ | +++ | ++ | +++ | ++ | ND | ND |
| 175 | ++++ | ++++ | ++ | +++ | +++ | +++ | ++ | +++ | ++ | + | ++++ |
| 176 | ++++ | ++ | + | ++ | ++ | ++ | ++ | +++ | + | ND | ND |
| 177 | ++++ | ++++ | ++ | +++ | +++ | +++ | +++ | +++ | + | + | ++++ |
| 178 | ++++ | ++++ | ++ | +++ | +++ | +++ | +++ | +++ | ++ | ND | ND |
| 179 | +++ | ++++ | ++ | +++ | ++ | ++ | +++ | +++ | + | + | +++ |
| 180 | ++ | ++ | + | + | ++ | + | + | ++ | + | ND | ND |
| 181 | ++ | ++++ | ++++ | ++ | ++++ | ++++ | ++ | ++++ | ++++ | + | ++++ |
| 182 | ++ | ++++ | +++ | ND | ND | ++++ | ++ | +++ | + | + | +++ |
| 183 | ++ | ++++ | ++++ | +++ | +++ | ++++ | +++ | +++ | + | + | +++ |
| 184 | + | ++++ | +++ | ++ | +++ | ++++ | ++ | ++++ | + | + | +++ |
| 185 | + | ++++ | ++ | ++ | +++ | ++++ | + | ++++ | + | + | +++ |
| 186 | +++ | ++++ | +++ | ++++ | ++ | ++++ | +++ | +++ | ++ | + | ++++ |
| 187 | ++++ | + | +++ | ND | ND | +++ | ND | ND | + | ND | ND |
| 188 | ++++ | ++ | ++ | +++ | +++ | ++ | +++ | ++ | ++ | ND | ND |
| 189 | ++++ | ++++ | ++ | +++ | +++ | ++ | ++ | +++ | + | ND | ND |
| 190 | ++++ | +++ | ++ | ++ | +++ | ++ | ++ | +++ | ++ | + | ++++ |

As noted in Table 8 above, in addition to activation of the ncNFkB signaling pathway, the activity of SMACm can also promote cell death in otherwise sensitive cells. Degradation of cIAP1/2 also affects intracellular signaling from TNFR family members, such as occurs after TNFa engagement with TNFR1. cIAP1/2 attach K48-linked ubiquitin chains to RIPK1 to facilitate recruitment of NEMO, which promotes activation of the canonical NFkB pathway. In the absence of cIAP1/2, TNFa ligation to TNFR1 instead leads to activation of the kinase activity of RIPK1, which leads to apoptosis or necroptosis. Binding of SMAC to the BIR domains of XIAP and ML-IAP antagonizes the caspase inhibition activities of these molecules, often overexpressed in tumor cells, leading to potentiation of apoptosis.

Most SMACm compounds developed over the past 20 years in the oncology space have been designed to kill tumor cells by specifically binding to and inhibiting XIAP by binding to the BIR2 and BIR3 domains in XIAP and in some cases have attempted to limit cIAP1/2 engagement. However, for the purposes of HIV latency reversal through the activation of ncNF-kB, cIAP1/2 binding is the preferred activity and XIAP inhibition is an off-target and may contribute in heretofore unknown mechanisms to potential cytotoxicity or in vivo toxicity. It is noted that bivalent SMACm can more optimally activate ncNF-kB than monovalent compounds, perhaps through bridging cIAP1 and cIAP2 together in a ternary complex where each SMACm motif in the bivalent molecule interacts with one BIR domain from each of cIAP1 and cIAP2, leading to the efficient degradation of both cIAP1 and cIAP2. Bivalent SMACm may also interact with cIAP1, cIAP2, and XIAP through intramolecular binding to each of the BIR2 and BIR3 domains within the same molecule. The bivalent molecules of Table 1 are optimized to bind to BIR3 more potently than BIR2 and as such may form the ternary complex between molecules as opposed to the intramolecular binding to BIR2 and BIR3, thereby favoring the depletion of cIAP1 and cIAP2 over inhibition of XIAP. The novel bivalent SMACm compounds of Table 1 (Compounds 1-180) are optimized to bind the BIR3 domains of the IAP family to disfavor intramolecular BIR2-BIR3 binding, promote ternary complex formation and activation of ncNF-kB, while limiting XIAP binding.

In some embodiments, the compounds were optimized to have potency in the Jurkat latency reversal assay presented in Table 8 indicated by the Jurkat assay potency pEC50 values. In some embodiments, the compound or pharmaceutically acceptable salt or stereoisomer thereof is selected from the group consisting of Compounds 2, 3, 16, 18, 20, 24-27, 29, 36, 49, 50-70, 72-94, 100, 106-113, 118-119, 122, 124, 129, 130-132, 134, 141-142, 145, 147-148, 150, 161-163, 165, 167-171, and 174-179. In other embodiments, the compound or pharmaceutically acceptable salt or stereoisomer thereof is selected from the group consisting of Compounds 27, 29, 36, 50-52, 54-55, 59, 64-70, 72-76, 79-82, 84-89, 91, 94, 100, 110, 113, 129, 161, 167-168, and 174-178.

In some embodiments, the compounds were optimized to have limited or no cytotoxicity as presented in Table 8 indicated by the Jurkat assay toxicity pCC₅₀. In some embodiments, the compound or pharmaceutically acceptable salt or stereoisomer thereof is selected from the group consisting of Compounds 1-24, 27, 29-30, 32-49, 57, 59, 63-65, 68-72, 74-78, 80-84, 86-93, 95-99, 101-106, 111-141, 143-146, 150-169, 171-175, and 177-179. In other embodiments, the compound or pharmaceutically acceptable salt or stereoisomer thereof is selected from the group consisting of Compounds 1-3, 5-23, 30, 32-35, 37-39, 41-45, 47-49, 57, 63, 71, 75, 77, 80-84, 89-93, 95-99, 101-106, 111-114, 116-119, 121-141, 143, 146, 154-169, 171-172, 175, and 177-179.

In some embodiments, the compounds were optimized to increased fold separation for BIR3 as presented in Table 8 indicated by cIAP1 BIR3 binding assays pIC₅₀. In some embodiments, the compound or pharmaceutically acceptable salt or stereoisomer thereof is selected from the group consisting of Compounds 3, 6, 15, 17-19, 22-23, 25, 45, 49-52, 63-65, 67, 69, 71-80, 84-88, 90-94, 96-107, 111-114, 118-127, 129-130, 136-141, 145-146, 150, 152, 154-155, 157-167, 169-172, and 174. In other embodiments, the compound or pharmaceutically acceptable salt or stereoisomer thereof is selected from the group consisting of Compounds 15, 17-19, 23, 49-52, 74-80, 90, 92, 96-99, 101, 104, 106, 119, 130, 138, 146, 155, 161-164, 170, and 171.

In some embodiments, the compounds were optimized to increased fold separation for BIR2 as presented in Table 8 indicated by cIAP1 BIR2 binding assays pIC₅₀. In some embodiments, the compound or pharmaceutically acceptable salt or stereoisomer thereof is selected from the group consisting of Compounds 1-3, 6-28, 34, 39, 49, 63, 67, 69-72, 74-75, 77, 79-80, 84, 86-88, 90-93, 95-98, 100, 104, 106, 107, 111-114, 118-122, 124-127, 129, 130-131, 133-134, 136-141, 154-158, 159-167, 171, 175, and 177-179. In other embodiments, the compound or pharmaceutically acceptable salt or stereoisomer thereof is selected from the group consisting of Compounds 3, 6-10, 12-15, 17, 19, 22, 25, 72, 75, 91, 93, 129, 159, 161-164, 166-167, and 171.

In some embodiments, the compounds were optimized to increased fold separation between BIR3 and BIR2 binding activity as presented in Table 8 indicated by cIAP1 BIR3 vs BIR2 separation window. In some embodiments, the compound or pharmaceutically acceptable salt or stereoisomer thereof is selected from the group consisting of Compounds 6, 18-19, 23, 28, 35-37, 40-41, 43-49, 55, 59, 63, 64, 67-70, 73-77, 79-80, 82, 83-90, 92, 96-107, 111-121, 123-128, 130, 132, 135-142, 144-155, 161, 165, 170, 172-175, 177, and 178. In other embodiments, the compound or pharmaceutically acceptable salt or stereoisomer thereof is selected from the group consisting of Compounds 55, 64, 82, 97, 146, and 148.

In some embodiments, the compounds were optimized to increased fold separation for BIR3 as presented in Table 8 indicated by cIAP2 BIR3 binding assays pIC₅₀. In some embodiments, the compound or pharmaceutically acceptable salt or stereoisomer thereof is selected from the group consisting of Compounds 6, 15, 17-19, 22-23, 25-27, 45, 49-52, 55, 58-59, 64, 66-71, 73-80, 84, 86-107, 109, 111-113, 118-141, 145-146, 150-152, 154-172, 174, 175, and 177. In other embodiments, the compound or pharmaceutically acceptable salt or stereoisomer thereof is selected from the group consisting of Compounds 6, 15, 17-19, 23, 25, 49-50, 64, 73-80, 90, 95, 97-98, 101, 104, 106-107, 119, 130, 138, 146, 159, 161-167, and 169-171.

In some embodiments, the compounds were optimized to increased fold separation for BIR2 as presented in Table 8 indicated by cIAP2 BIR2 binding assays pIC₅₀. In some embodiments, the compound or pharmaceutically acceptable salt or stereoisomer thereof is selected from the group consisting of Compounds 1-3, 5-27, 39, 65, 68-72, 74-77, 82, 86, 88-93, 95-96, 104, 106, 112, 118-122, 124, 126-127, 129-132, 134, 136-141, 154-156, 157-160, 162-164, 166-168, 170-171, and 177-179. In other embodiments, the compound or pharmaceutically acceptable salt or stereoisomer thereof is selected from the group consisting of Compounds 6-7, 10, 14-15, 17, 19-20, 22, 25, 65, 75, 90, 119, 157-159, 162-164, 166-168, and 171.

In some embodiments, the compounds were optimized to increased fold separation between BIR3 and BIR2 binding activity as presented in Table 8 indicated by cIAP2 BIR3 vs BIR2 separation window. In some embodiments, the compound or pharmaceutically acceptable salt or stereoisomer thereof is selected from the group consisting of Compounds 6, 15, 18-19, 23, 25-28, 35, 39-45, 47-49, 55, 59-60, 63-64, 67-77, 79-80, 82-93, 95-107, 111-141, 143-146, 150-152, 154-156, 159-163, 165-166, 170, 172, and 173-179. In other embodiments, the compound or pharmaceutically acceptable salt or stereoisomer thereof is selected from the group consisting of Compounds 44, 47-49, 60, 64, 101-102, 107, 143, 146, 150, 161, and 172.

In some embodiments, the compounds were optimized to increased fold separation for BIR3 as presented in Table 8 indicated by XIAP BIR3 binding assays pIC₅₀. In some embodiments, the compound or pharmaceutically acceptable salt or stereoisomer thereof is selected from the group consisting of Compounds 6, 15, 17-19, 22-23, 25, 49-50, 64-65, 67-68, 74-75, 78-79, 80, 87-88, 90-92, 95-99, 101-107, 118-119, 121, 123-124, 126-127, 130, 138, 154, 158-161, and 163-164. In other embodiments, the compound or pharmaceutically acceptable salt or stereoisomer thereof is selected from the group consisting of Compounds 6, 15, 17-19, 23, 49-50, 74-75, 80, 104, and 106.

In some embodiments, the compounds were optimized to increased fold separation for BIR2 as presented in Table 8 indicated by XIAP BIR2 binding assays pIC₅₀. In some embodiments, the compound or pharmaceutically acceptable salt or stereoisomer thereof is selected from the group consisting of Compounds 1, 2, 7, 10, 31, 32, and 33.

In some embodiments, the compounds were optimized to increased fold separation between BIR3 and BIR2 binding activity as presented in Table 8 indicated by XIAP BIR3 vs BIR2 separation window. In some embodiments, the compound or pharmaceutically acceptable salt or stereoisomer thereof is selected from the group consisting of Compounds 3,4-6, 8-9, 11-12, 15-19, 21-24, 27-28, 34-35, 37-39, 41-45, 47-49, 60, 63, 66, 68-72, 75, 77-80, 84-85, 87-88, 90-93, 95-107, 110-114, 117-118, 121-133, 134-146, 150, 154-160, 163, 166-167, 172, 175, 177, and 179. In other embodiments, the compound or pharmaceutically acceptable salt or stereoisomer thereof is selected from the group consisting of Compounds 4-6, 8, 11-12, 15-19, 22-23, 27, 37-38, 44-45, 47-49, 60, 63, 66, 69-71, 75, 79-80, 84-85, 87-88, 90-93, 95-107, 110, 113-114, 117-118, 121-133, 135-138, 140, 145-146, 150, 154-160, 163, 166-167, 172, 175, and 177.

In view of the combined potency, cytotoxicity, and increased fold separation between BIR3 and BIR2 binding activity, specifically with respect to cIAP1 and cIAP2 BIR3, in some embodiments, the compound or pharmaceutically acceptable salt or stereoisomer thereof is selected from the group consisting of Compounds 15, 18-19, 23, 49, 69-70, 74-75, 77, 79-80, 84, 86-88, 90-93, 96-98, 104, 106-107, 112, 118-119, 121-122, 124, 126-127, 129-130, 132, 136-141, 154-155, 159-161, 163, 177, 183, and 186. In other embodiments, in view of the combined potency, cytotoxicity, and increased fold separation between BIR3 and BIR2 binding activity, specifically with respect to cIAP1 and cIAP2 BIR3, the compound or pharmaceutically acceptable salt or stereoisomer thereof is selected from the group consisting of Compounds 18, 75, 90, 91, 92, 106, 119, 124, and 130.

### Pharmacokinetic Data

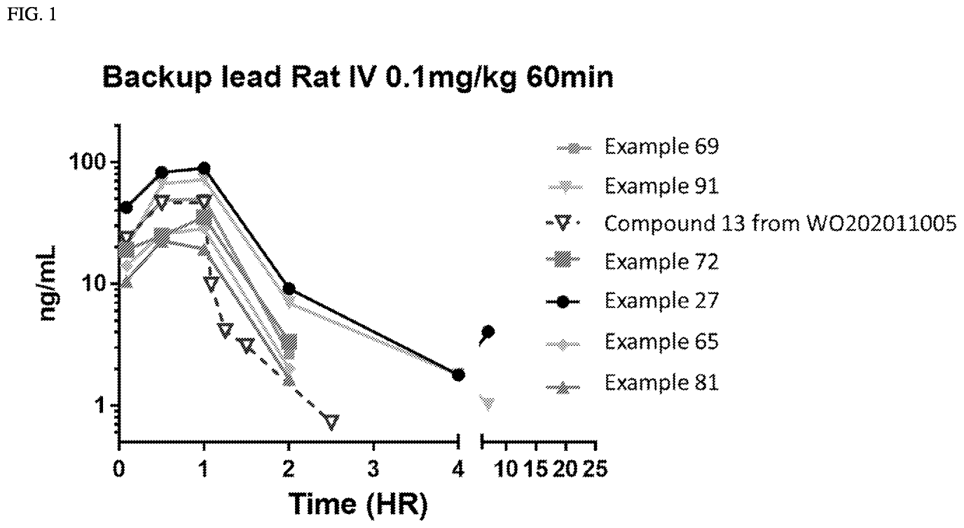

Rodent pharmacokinetic (PK) data of several SMAC mimetic compounds of Formula I was compared with that of SMACm AZD5582 PK data (FIG. 1). As can be seen in FIG. 1, the plasma drug concentration versus time curves for the compounds of Formula I demonstrate a PK relationship comparable to that of AZD5582 and is expected to have the comparable ability to translate to in vivo models of HIV latency.

### NFkB2 Gene Induction

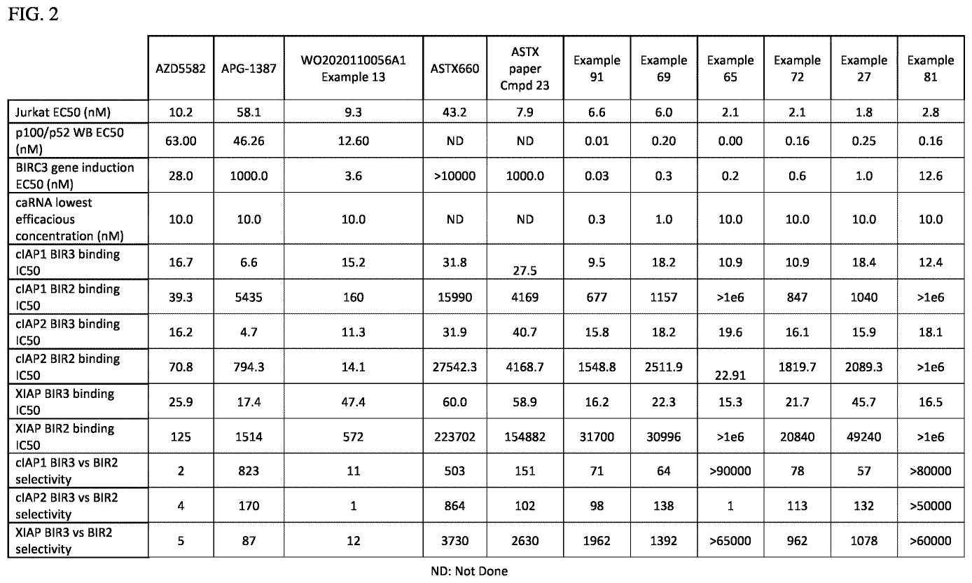

5×10⁵ normal donor CD4 T cells were treated for 24 h with serial dilutions of compounds starting at 1.0 μM at 6 fold, 7 places. Total RNA was isolated using the RNEasy Mini kit (Qiagen) per the manufacturer's instructions. The following TaqMan primer probe sets were sourced from Applied Biosystems: Hs00174517_ml (NFκB2) and Hs02800695 (HPRT1). TaqMan-based real-time PCR (Fast Virus 1-Step Master Mix, Applied Biosystems) was used to amplify host genes of interest. Gene expression was normalized to HIPRT1 and comparative threshold cycle (CT) method (ΔΔCT) was used for relative fold change of gene expression as compared to untreated cells. The data was analyzed by QuantStudio3 Real-Time PCR System. Results are provided in FIG. 2.

### p100/p52 Western Blot

### Cell Culture and Treatment

Cell cultures were set up to evaluate several SMACm compounds. Frozen PBMC cells from healthy donors were thawed and plated in 2 ml deepwell plates at 2×10⁶ cells/ml. Serial dilutions of each compound were carried out as a 7 fold 1:6 dilution with the starting final dose of 1000 nM. Cells were then combined with compound and place at 370 for 24 hrs. The following day cells were pelleted and placed at −80° until lysis.

### Harvest of Whole Cell Lysis

In preparation for cell lysis, the frozen cell pellets and an aliquot of NP40 Cell Lysis Buffer (Invitrogen, Part #FNN0021), as well as an aliquot of 10× Protease Inhibitor Cocktail (Sigma, Part #P-2714) are thawed on ice. Once thawed, the complete lysis buffer is prepared by adding 10% final volume PI cocktail to the NP40 buffer and then adding in sufficient volume of 0.1 M PMSF (Sigma, Part #93482) and 1 M DTT (Sigma, Part #43816) to create a final concentration of 1 mM each in the complete lysis buffer. Cell lysis is then carried out by resuspending each cell pellet in 30 L per 10\hat{ }6 cells then incubating on ice for 30 minutes with vigorous vortexing every 10 minutes. The lysed cells are then centrifuged at 13K RPM for 10 minutes at 4° C. in a refrigerated microcentrifuge. The supernatants (lysates) are then transferred into new microcentrifuge tubes and either stored at −80° or utilized immediately for protein concentration and Western blot analysis.

### Bradford Microplate Procedure

Protein concentration for each lysate is determined using a detergent compatible Bradford assay (Pierce, Part #23246) following the manufacturer's microplate instructions.

### Protein Separation and Immunodetection

Capillary Western analysis were performed using the ProteinSimple Jess System. A total of lug of cell lysate was prepared according to the provided protocol supplied by ProteinSimple. The primary antibody for p100/p52 (Cell, Signaling #3017) is diluted 1:10 for use in the Jess system. The 12-230 kDa Jess Separation Module capillary cartridges Separation Module (SM-W004) is used in conjunction with the Anti-Rabbit Secondary NIR conjugate (043-819). The Separation module includes prefilled plates that the samples, primary and secondary antibodies, as well as protein normalization reagent and diluents are loaded onto to perform separation and detection. Once the assay has run, the data is then analyzed utilizing the Compass software provided by ProteinSimple.

### Immunoblot Analysis

Alternatively to capillary electrophoresis, traditional immunoblot assays were carried out, 10 μg of cell lysate was loaded per well into 4-20% Tris-Glycine SDS-PAGE gels. Protein from the SDS-PAGE gels were transferred to Turbo Midi PVDF Transfer Packs (BioRad) using the “Mixed MW” protocol for one Midi Format Gel (constant 2.5A up to 25V, for 7 minutes) of the Trans-Blot Turbo Transfer System (Bio-Rad) with premade Trans-Blot per the manufacturer's instructions. After transfer, PVDF membranes were blocked in 5% bovine serum albumin (BSA) in 1× TBS (BioRad) with 0.1% Tween-20 for 1 hour at room temperature with gentle rocking. Primary antibodies were added and incubated overnight at 4° C. (anti-p100/p52, Cell Signaling Technology #3017, 1:1000; anti-Actin-HRP conjugate, Abcam #49900, 1:30,000). Following primary staining, the membrane washed three times with 1× Tris Buffered Saline (TBS)+0.1% TWEEN® 20 for 10 minutes each wash. After washing, the membrane was incubated in 5% BSA in 1× TBS+0.1% TWEEN® 20 with the appropriate secondary antibody for 2 hours at room temperature. Following secondary stain the membrane was washed twice for 10 minutes with 1× TBS+0.1% TWEEN® 20 followed by a 10-minute wash with 1× TBS. The membrane was then patted dry with filter paper and an image was captured of the undeveloped membrane on the ChemiDoc MP Imaging System using Image Lab software (BioRad). Sufficient ECL reagent (GE Healthcare) was used to cover the membrane and a series of images were taken starting with 0.001 second and doubling to tripling the exposure time until the luminescence from the developed membrane saturated the image. The developed membrane was then washed three times with 1× TBS for 5 minutes to remove the residual ECL reagent and then stored at 4° C. in sufficient 1× TBS to submerse the entire membrane. Densitometry of images of the developed membrane were then carried out using the Image Lab software. Some membranes were stripped for one minute with One Minute Plus Western Blot Stripping Buffer (GM Biosciences) and then washed three times for 10 minutes with 1× TBS. The stripped membranes were then blocked in 5% BSA in 1× TBS+0.1% TWEEN® 20 for an hour and reprobed overnight with a new primary antibody. Results of the p100/p52 WB line assay are provided in FIG. 2.

### Cell Associated HIV RNA (caRNA)

During the productive phase of the viral life cycle, HIV produces a large number of differentially spliced transcripts collectively termed cell-associated HIV RNA (ca-HIV RNA) within some cells that are infected. In HIV-infected individuals on suppressive antiretroviral therapy (ART), changes in the levels of caHIV RNA is an accepted surrogate measure of the efficacy of HIV latency reversal.

To measure ca-HIVRNA, peripheral blood mononuclear cells (PBMCs) are isolated from leukocytes obtained by continuous-flow leukapheresis. Total CD4⁺ T cells were isolated from PBMCs by negative selection using the EasySep Human CD4⁺ T cell Enrichment kit (StemCell, Vancouver, Canada) per the manufacturer's recommendations. Resting CD4⁺ T cells are isolated by negative selection with an immunomagnetic column as described previously (25) and either cryopreserved immediately or maintained for two days in IMDM medium (Gibco), 10% FBS, 2 μg/mL IL-2 (Peprotech), and antiretrovirals to prevent viral expansion.

For SMACm treatment, three to five replicates of 2-5×10⁶ CD4⁺ T cells were treated for 48 hours at 37° C. in 1 mL of RPMI medium 1640, 10% FBS, 10 μg/mL of enfuvirtide (Sigma), and 200 nM rilpivirine. 10 nM phorbol 12-myristate 13-acetate (PMA; Sigma) with 1 μM ionomycin (Sigma) was used as a positive control for LRA activation and 0.2% DMSO vehicle was used as a negative control. Following treatment, cells were lysed and RNA and DNA were co-extracted using an AllPrep 96 RNA/DNA kit (Qiagen, Valencia, CA) per the manufacturer's instructions, adjusting the volume of lysis buffer to 0.6 mL, adding an on-column DNase I treatment (Qiagen) and eluting RNA in 50 μL of water.

RT-qPCR was performed in triplicate for each of three replicate wells using TaqMan Fast Virus 1-step RT-qPCR Master Mix (Applied Biosciences) with 5 μL isolated RNA and 900 nM of HIV capsid primers HIV-gag (5′-ATCAAGCAGCTATGCAAATGTT-3′ (SEQ ID NO: 1)) and gag reverse (5′-CTGAAGGGTACTAGTAGTTCCTGCTATGTC-3′ (SEQ ID NO: 2)) and 250 nM of FAM/ZEN/IABFQ HIV gag probe (5′-ACCATCAATGAGGAAGCTGCAGAATGGGA-3′ (SEQ ID NO: 3)). Samples were amplified and data was collected using a QuantStudio™ 3 Real-Time PCR system (Applied Biosystems) with the following cycling conditions: one cycle at 50° C. for 5 minutes (reverse transcription), one cycle at 95° C. for 20 seconds (reverse transcriptase inactivation), and 50 cycles at 95° C. for 3 seconds and 60° C. for 20 seconds (denaturation and annealing/extension).

HIV absolute HIV gag RNA copies per reaction were determined using an HIV gag gBlock qPCR standard corresponding to the DNA sequence of the qPCR product (5′-ATCAAGCAGCCATGCAAATGTTAAAAGAGACCATCAATGAGGAAGCTGCAGA ATGGGATAGATTGCATCCAGTGCATGCAGGGCCTATTGCACCAGGCCAGATGA GAGAACCAAGGGGAAGTGACATAGCAGGAACTACTAGTACCCTTCAG-3′ (SEQ ID NO: 4); Integrated DNA Technologies), and copies were normalized to cell counts as determined by bright field microscopy. RT-qPCR efficiency was required to be between 90% to 110%. Assay values with a positive signal that were less than the lower limit of detection (LLOD) of seven HIV gag copies per reaction were adjusted to the LLOD. Analysis was performed using QuantStudio™ Design and Analysis Software (Applied Biosystems) and the R software package (described in detail below).

Provided in FIG. 2 are data comparing the Jurkat EC50, p100/p52 WB assay, NFkB2 gene induction assay and caRNA assay data of several SMAC mimetics disclosed herein with known mimetics. To help exemplify the potency, selectivity, and toxicity advantages of the inventive compounds disclosed herein over those compounds in the art, the data for several known compounds in the art is provided for comparison. The chemical structures for AZD5582, Example 13 of the WO 2020/110056, ASXT660, APG-1387, and compound 23 of the J. Med. Chem. 2018, 61, 7314 is shown below:

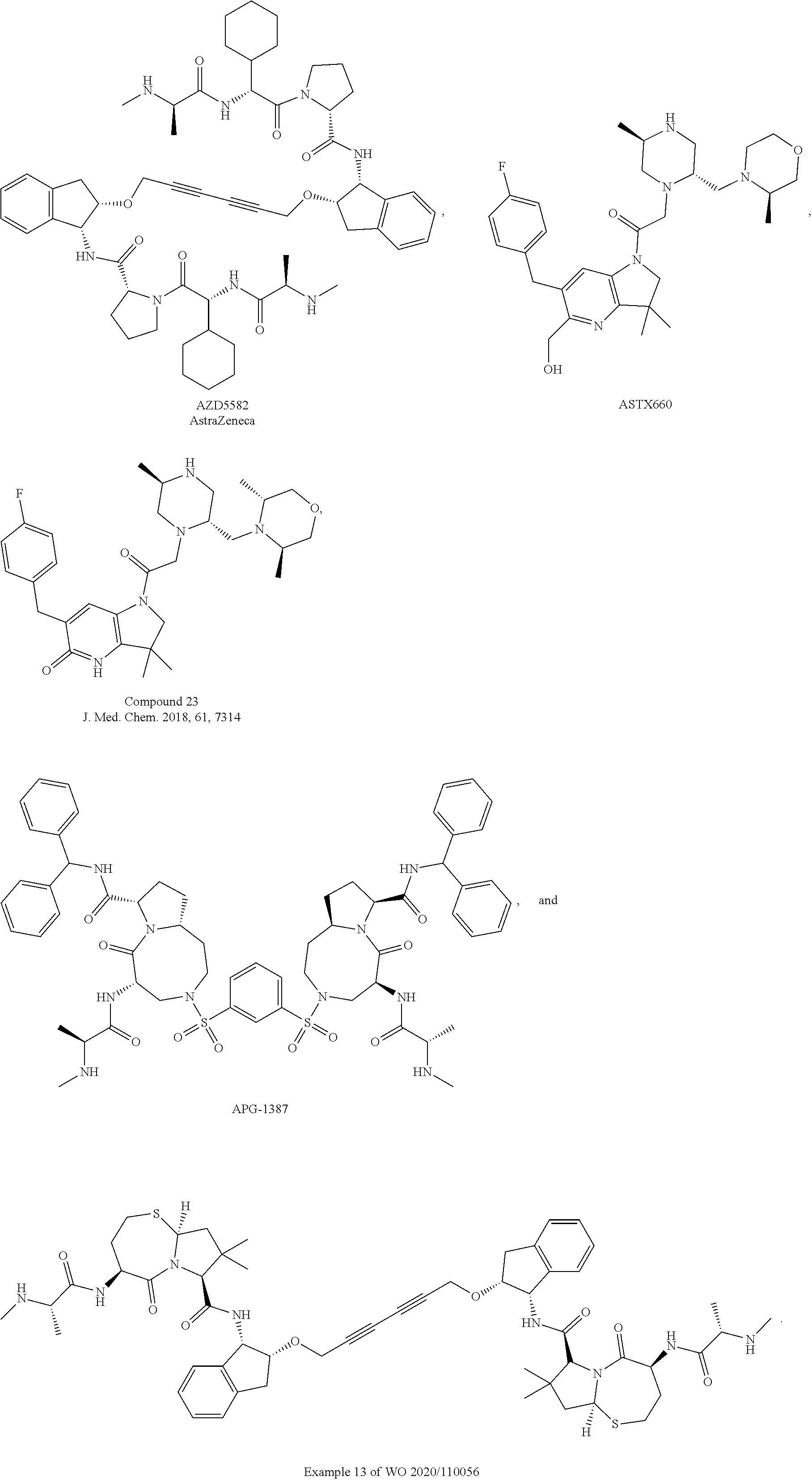

As exemplified in FIG. 2 for select representative compounds, not to be limiting, the compounds of the present invention are potent dimeric SMACm which are able to activate the ncNF-kB pathway and induce HIV expression. These molecules efficiently deplete cIAP1 and cIAP2, lead to cleavage of p100 to release p52, activate the ncNF-kB pathway and induce HIV expression. Moreover, these have properties that engage the ncNF-kB pathway in vivo. For example, in some embodiments, Examples 72 and 81 demonstrate preferred BIR3, cIAP1, cIAP2, and XIAP selectivity compared to their selectivity to BIR2. These compounds also demonstrate the preferred selectivity for BIR over BIR3, for either or both of the cIAP1 and XIAP proteins, theorized to favor ternary complex formation over bivalent intramolecular BIR2-BIR3 engagement. In some embodiments, the compound or pharmaceutically acceptable salt or stereoisomer thereof is selected from the group consisting of Compounds 27, 65, 69, 72, 81, and 91.

Efforts have been made to ensure accuracy with respect to numbers used (e.g. amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A compound of Formula (I): 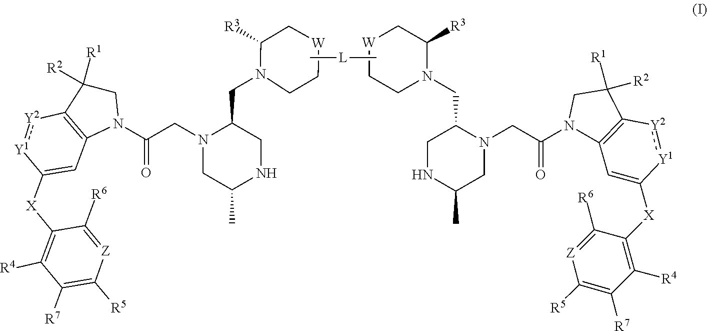
or a pharmaceutically acceptable salt thereof, wherein:
each R¹ and R² is —H, or —CH₃;
each R³ is —H or —CH₃;
each R⁴ is —H, —F, —Cl, —CH₃, —CF₃, —CN, —OH, —OCH₃, —C(O)N(CH₃)₂, —CH(CH₃)₂, or —C(O)OCH₃;
each R⁵ is —H, —F, —Cl, —Br, —CH₃, —CHF₂, or —CF₃;
each R⁶ is —H, —F, or —Cl;
each R⁷ is —H or —F;
each W is —CH—, —CH₂—, —O—, or —N—;
each X is —CH₂—, —O—, —NH—, or —NCH₃—;
each Y¹ is —CH— or —C(O)—;
each Y² is —N—, —NH—, or —NCH₃—;
each [symbol] represents a single or double bond, wherein when Y¹ is —CH—, Y² is —N— and [symbol] represents a double bond and when Y¹ is —C(O)—, Y² is —NH— or —NCH₃—, and [symbol] represents a single bond;
each Z is —CH—, —CF— or —N—; and
L is a linker selected from the group consisting of 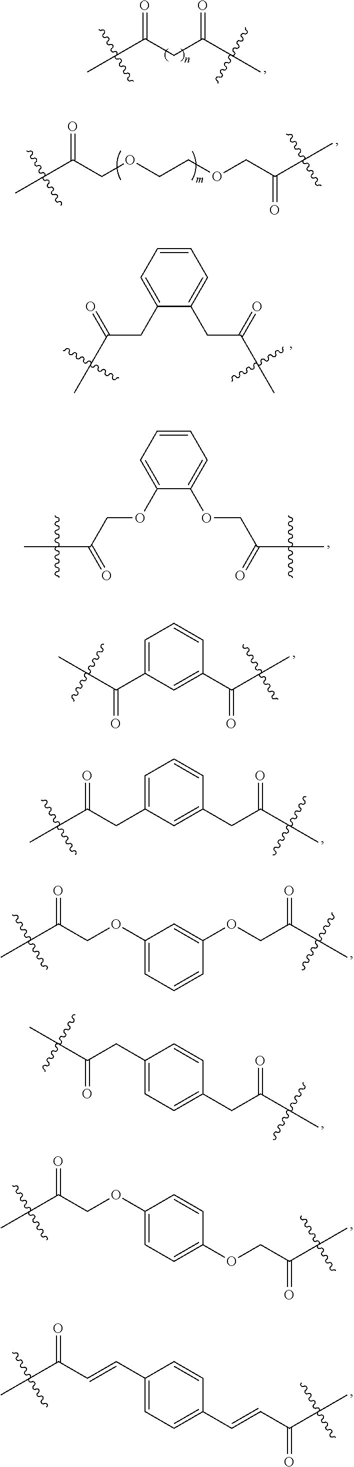 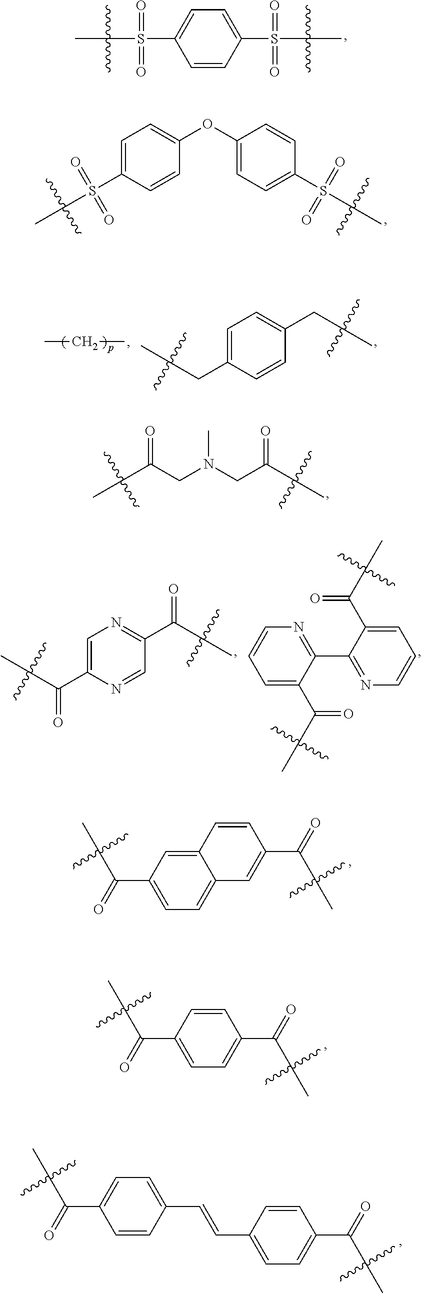 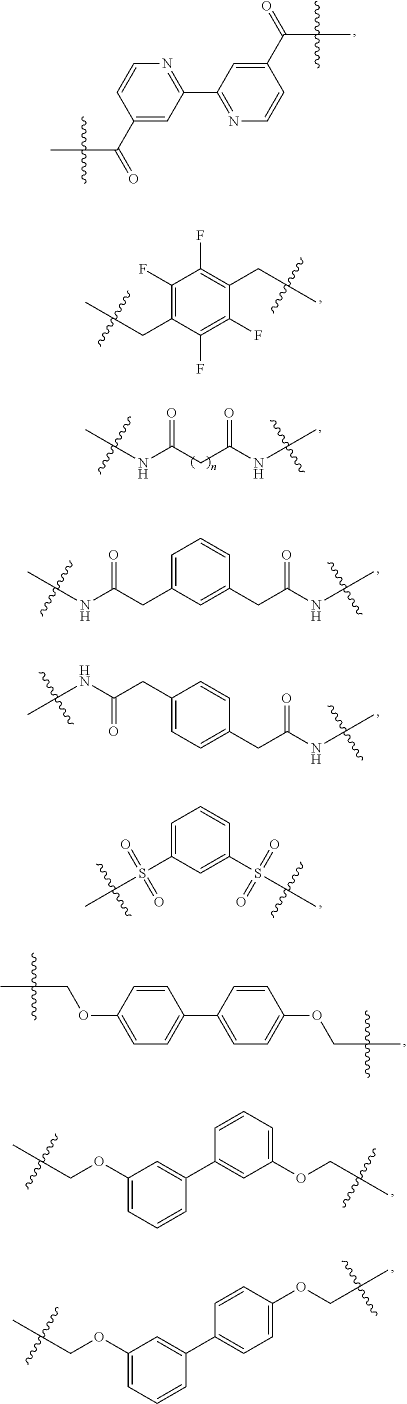 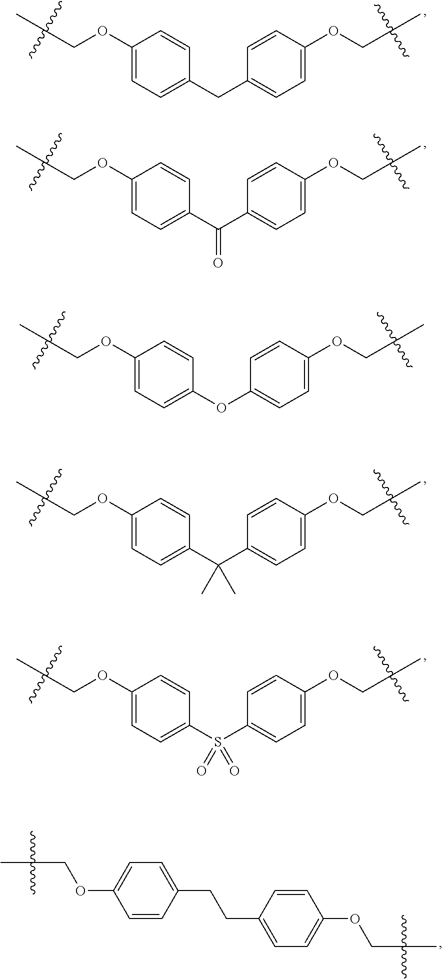 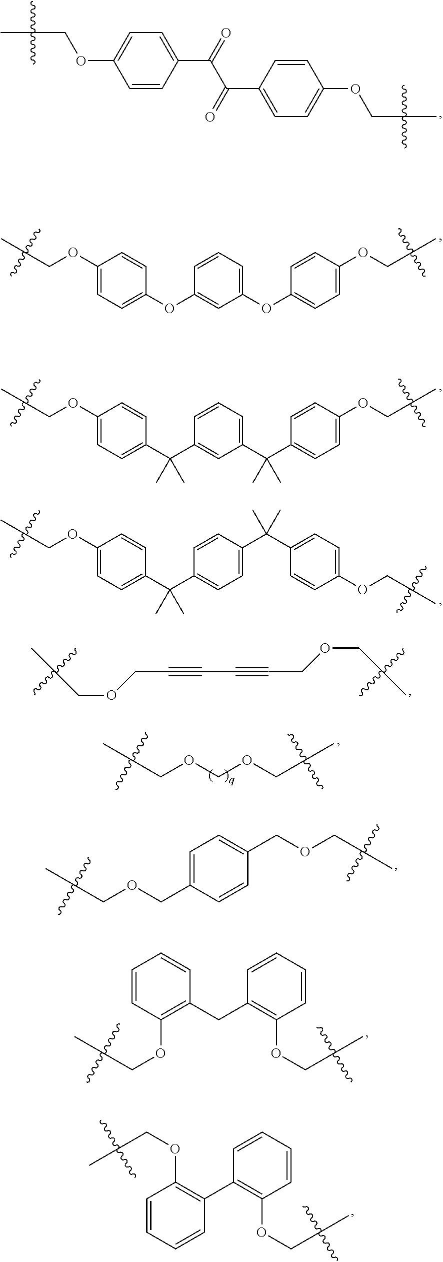 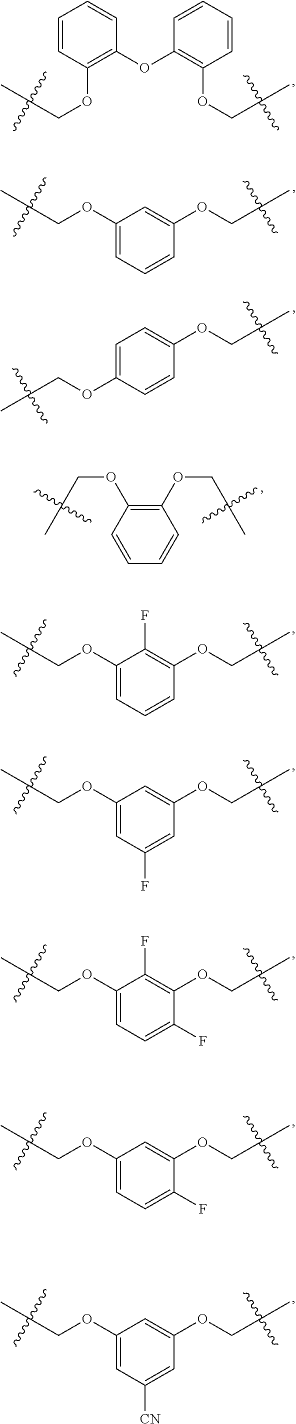 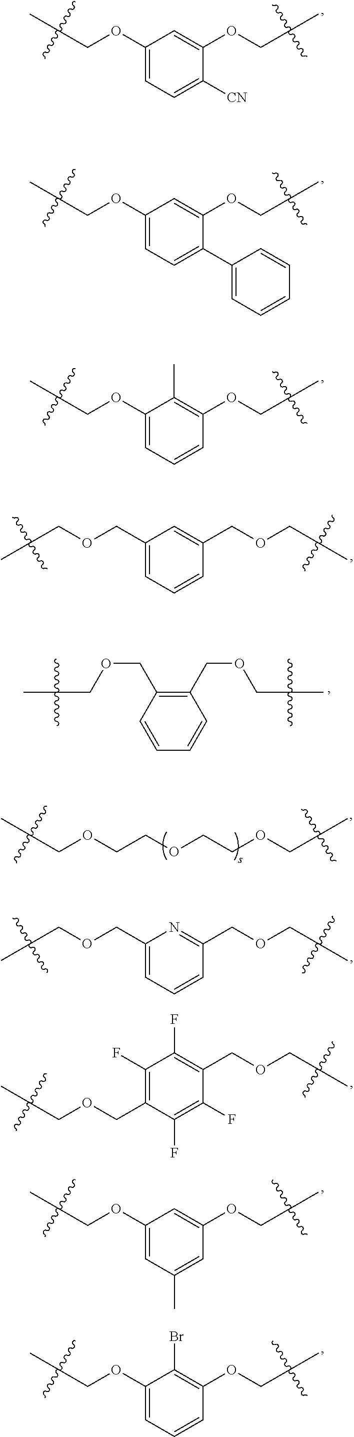 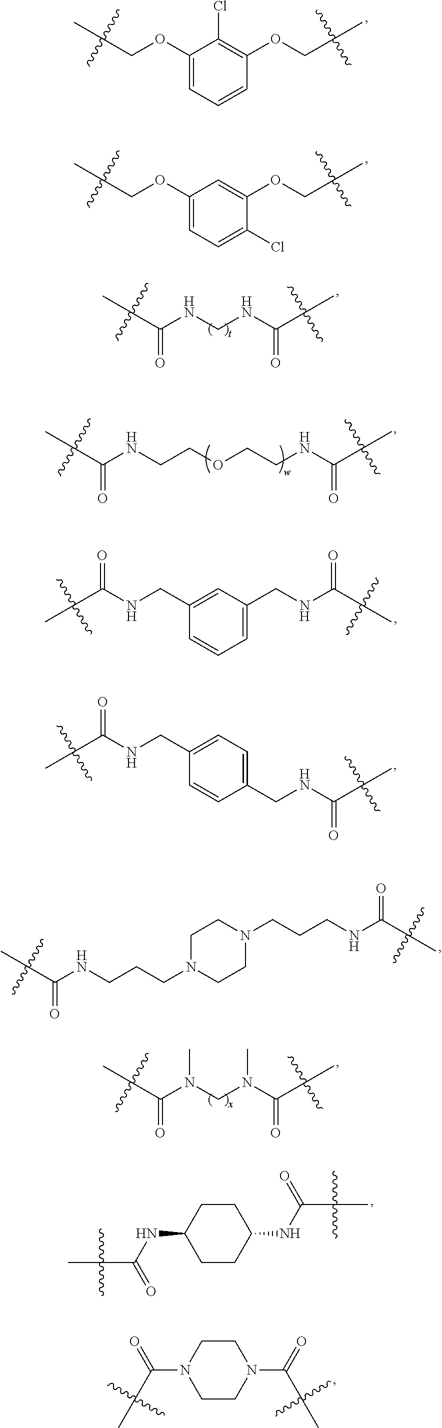 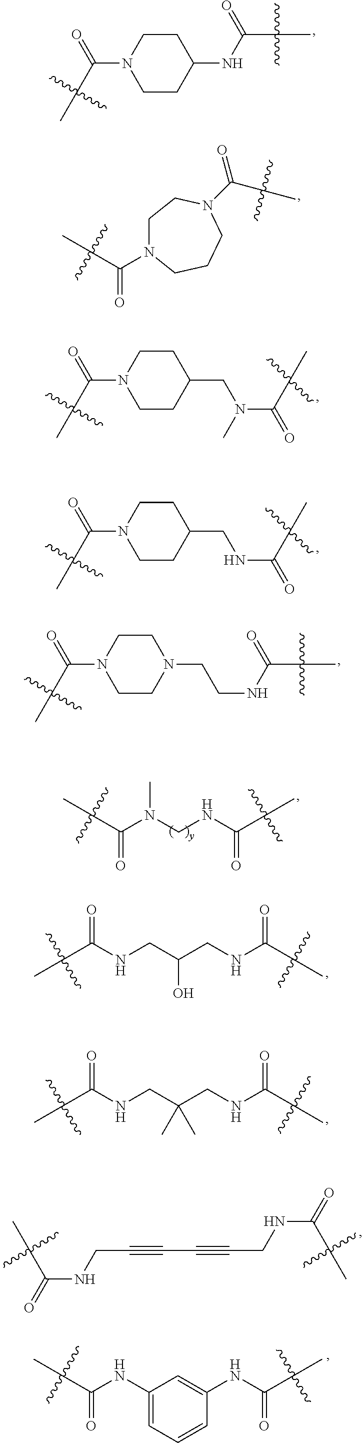 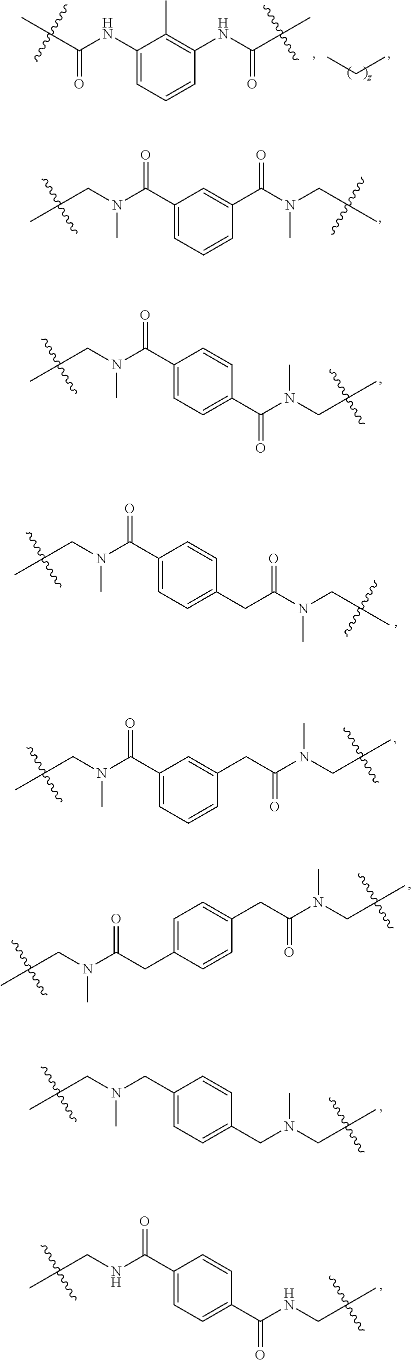 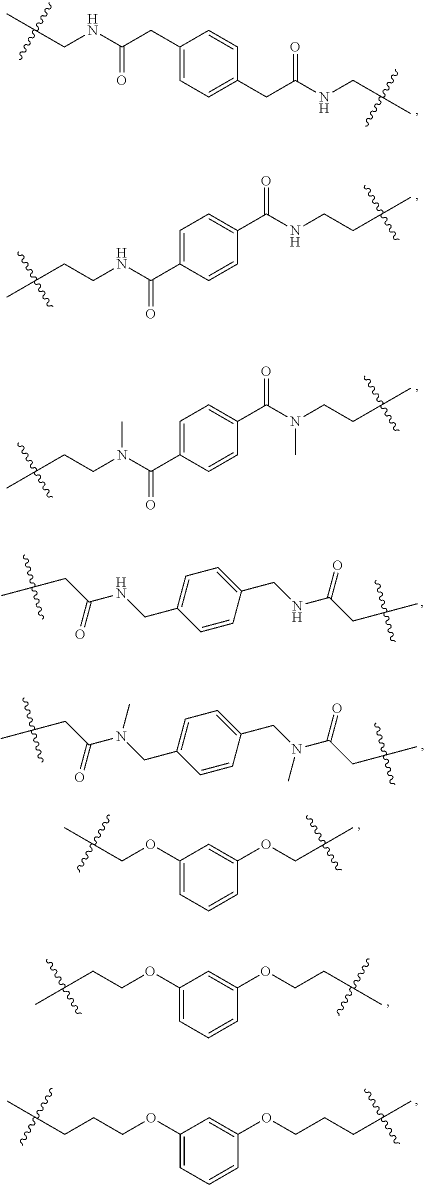 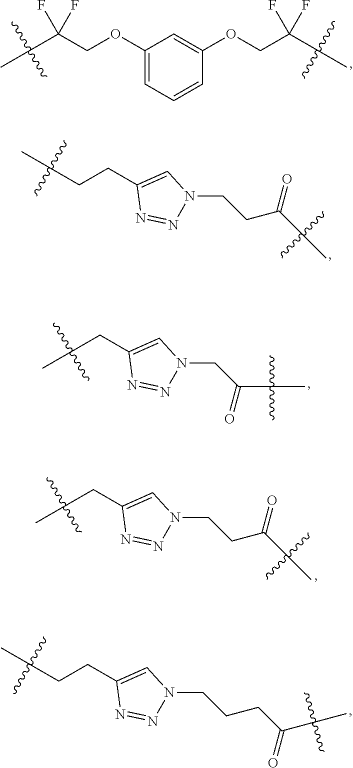 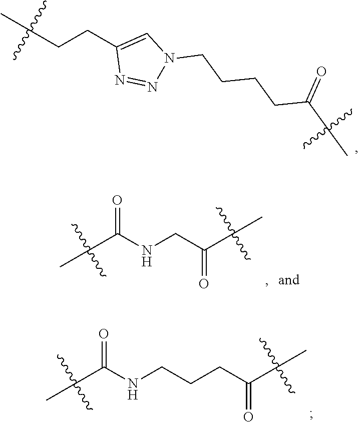
wherein n is an integer from 2 to 15, m is an integer from 1 to 5, p is an integer from 1 to 20, q is an integer from 2 to 15, s is an integer from 1 to 8, t is an integer from 2 to 15, w is an integer from 1 to 10, x is an integer from 2 to 15, y is an integer from 2 to 15, and z is an integer from 2 to 15.

2. The compound or pharmaceutically acceptable salt thereof according to claim 1, wherein R¹ and R² in each instance are each —CH₃.

3. The compound or pharmaceutically acceptable salt thereof according to claim 1, wherein each X is —CH₂—, each Z is —CH—, and each R⁴ is —H.

4. The compound or pharmaceutically acceptable salt thereof according to claim 1, wherein each Y¹ is —C(O)—, each Y² is —NH— or —NCH₃—, and each [symbol] represents a single bond.

5. A compound which is 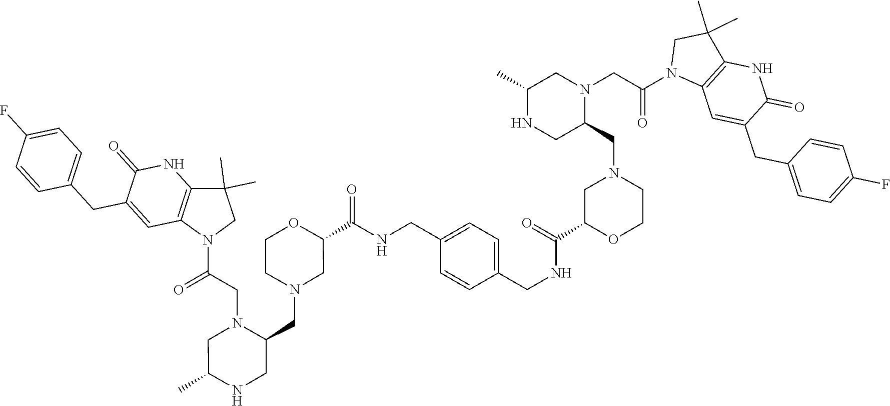 [SMILES: C[C@@H]1CN(CC(=O)N2CC(C)(C)c3[nH]c(=O)c(Cc4ccc(F)cc4)cc32)[C@@H](CN2CCO[C@H](C(=O)NCc3ccc(CNC(=O)[C@@H]4CN(C[C@H]5CN[C@H](C)CN5CC(=O)N5CC(C)(C)c6[nH]c(=O)c(Cc7ccc(F)cc7)cc65)CCO4)cc3)C2)CN1]
or a pharmaceutically acceptable salt thereof.

6. The compound or pharmaceutically acceptable salt thereof according to claim 1, wherein the compound is selected from the group consisting of

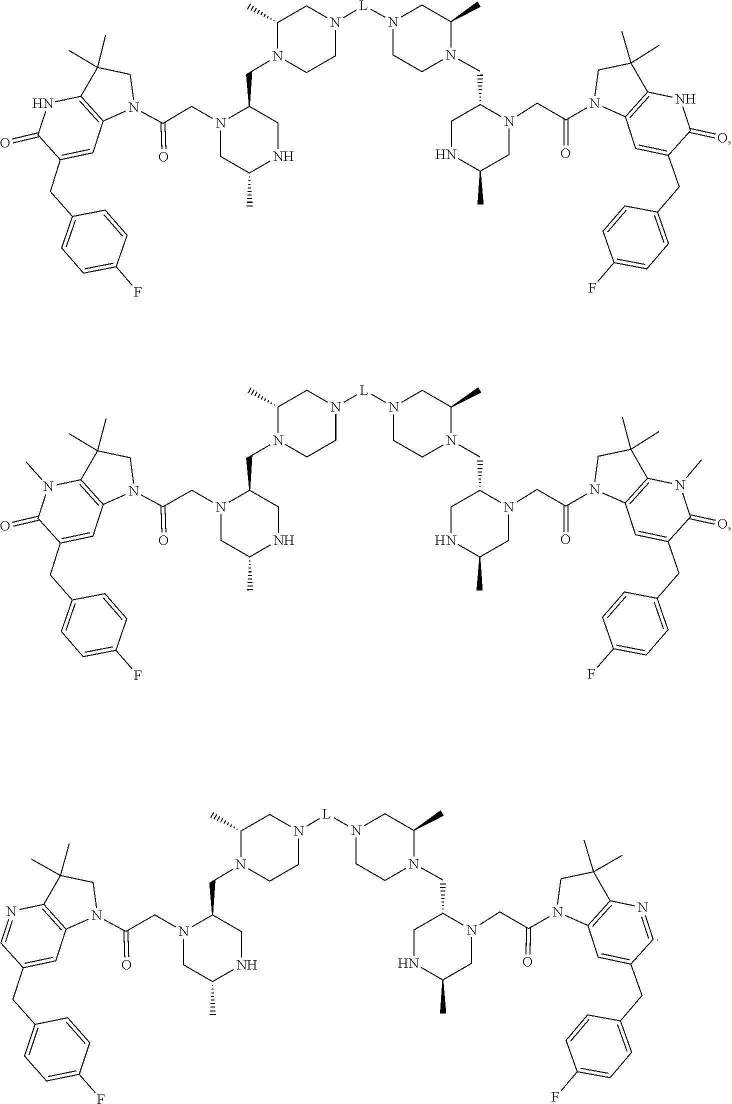

7. The compound or pharmaceutically acceptable salt thereof according to claim 6, wherein L is a linker selected from the group consisting of 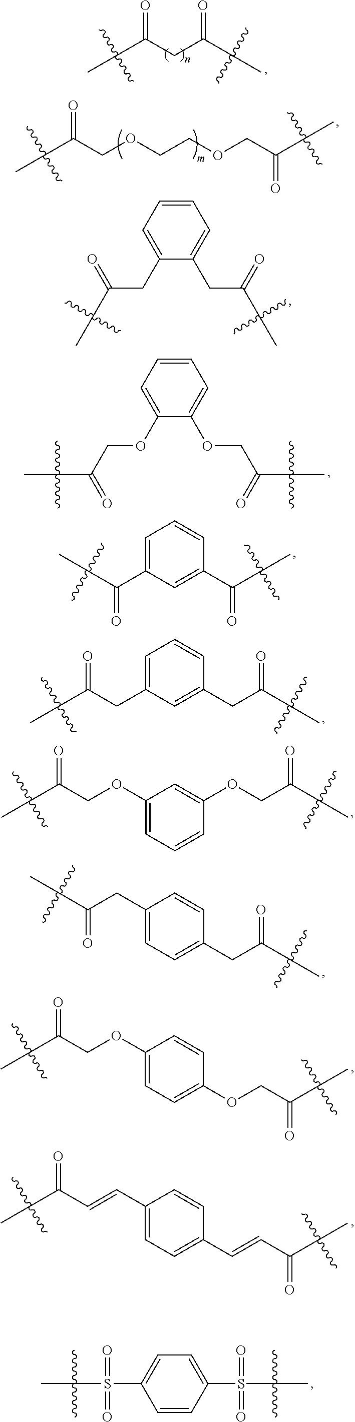 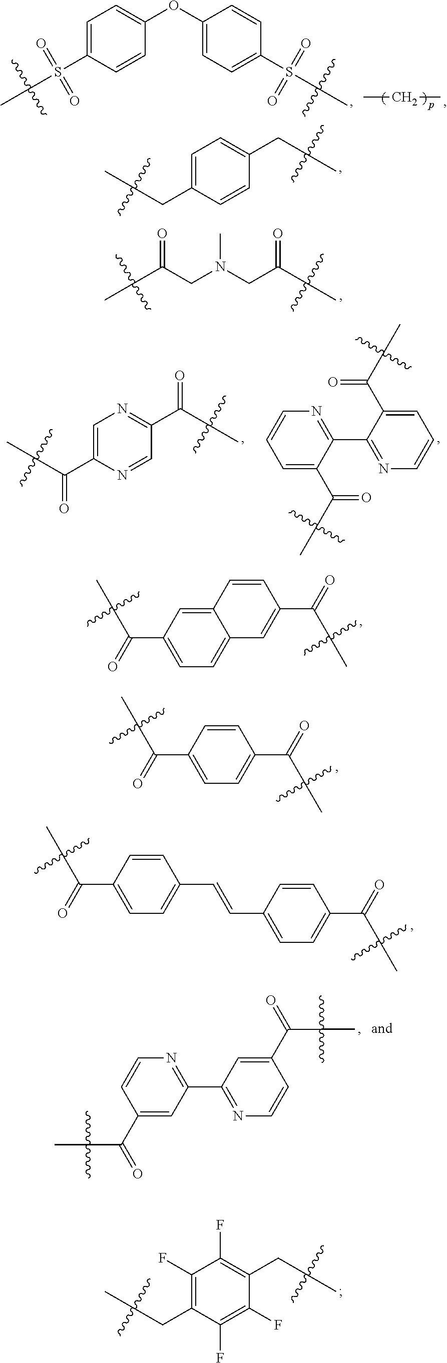
wherein n is an integer from 2 to 12, m is an integer from 1 to 3, and p is an integer from 1 to 12.

8. The compound or pharmaceutically acceptable salt thereof according to claim 1, wherein the compound is 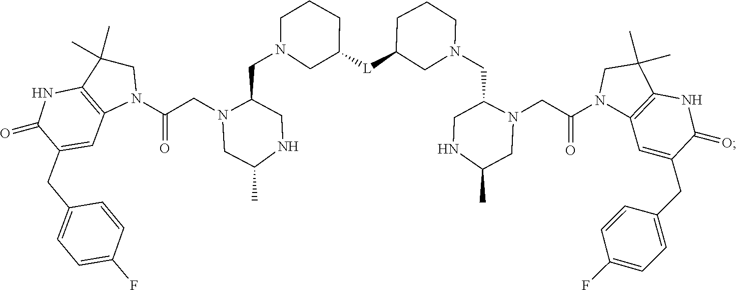
or a pharmaceutically acceptable salt thereof.

9. The compound or pharmaceutically acceptable salt thereof according to claim 8, wherein L is selected from the group consisting of: 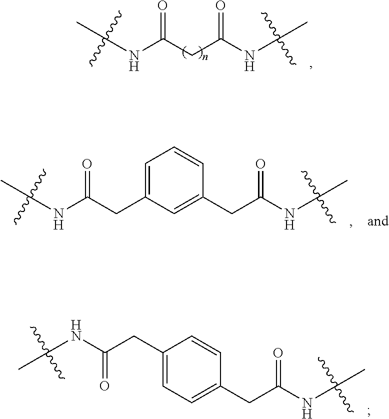
wherein n is an integer from 2 to 8.

10. The compound or pharmaceutically acceptable salt thereof according to claim 1, wherein the compound is selected from the group consisting of

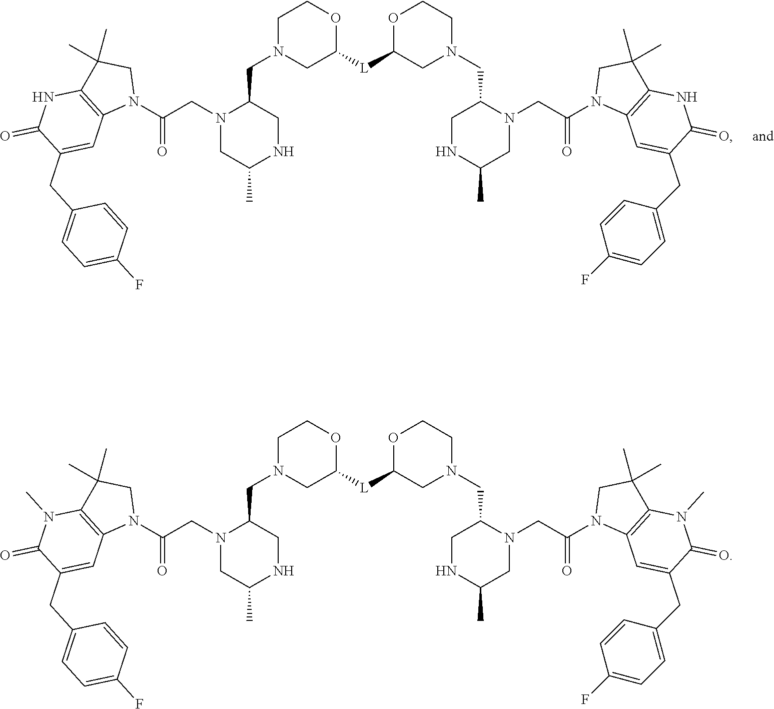

11. The compound or pharmaceutically acceptable salt thereof according to claim 10, wherein L is selected from the group consisting of 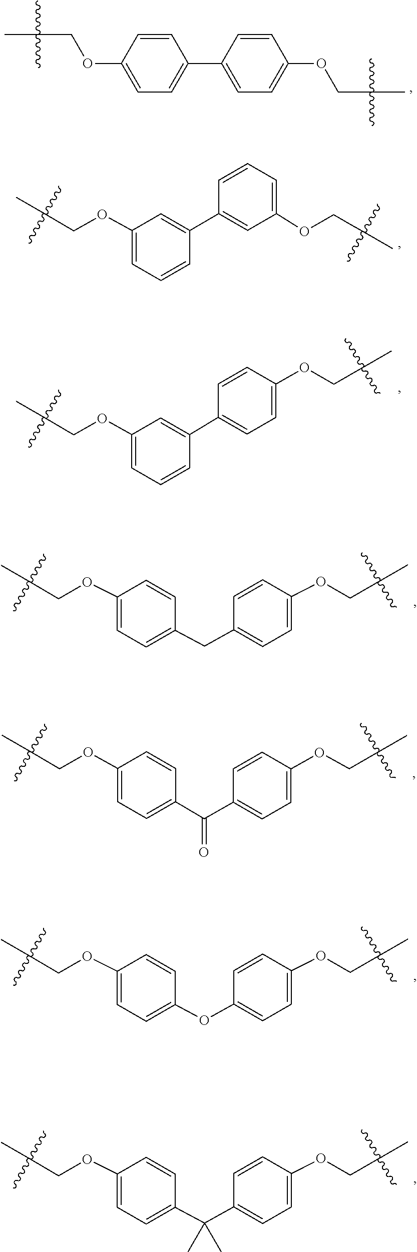 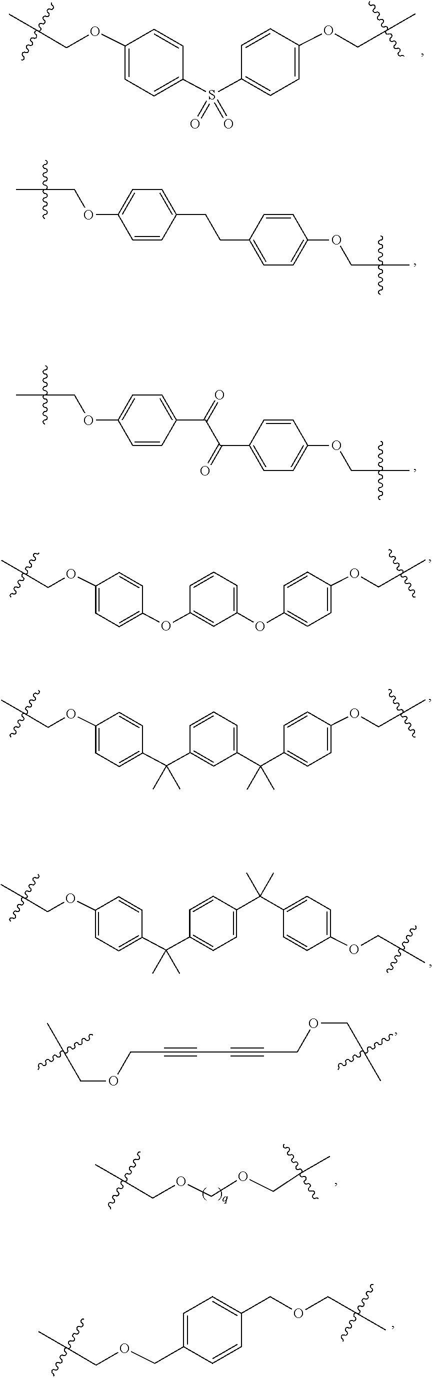 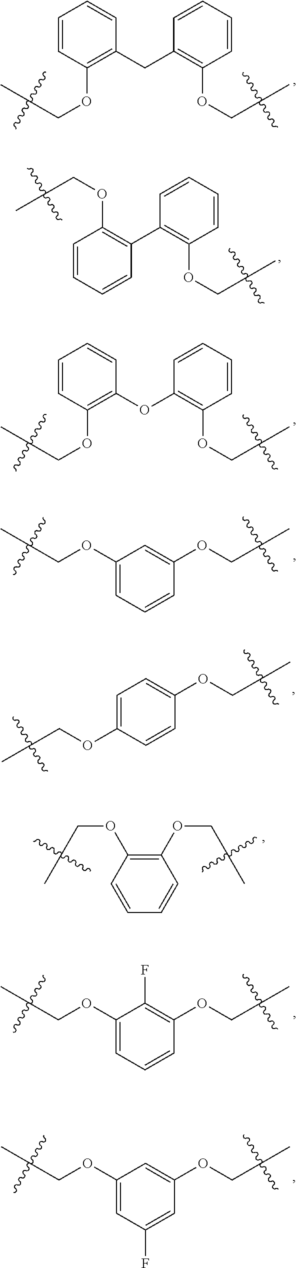 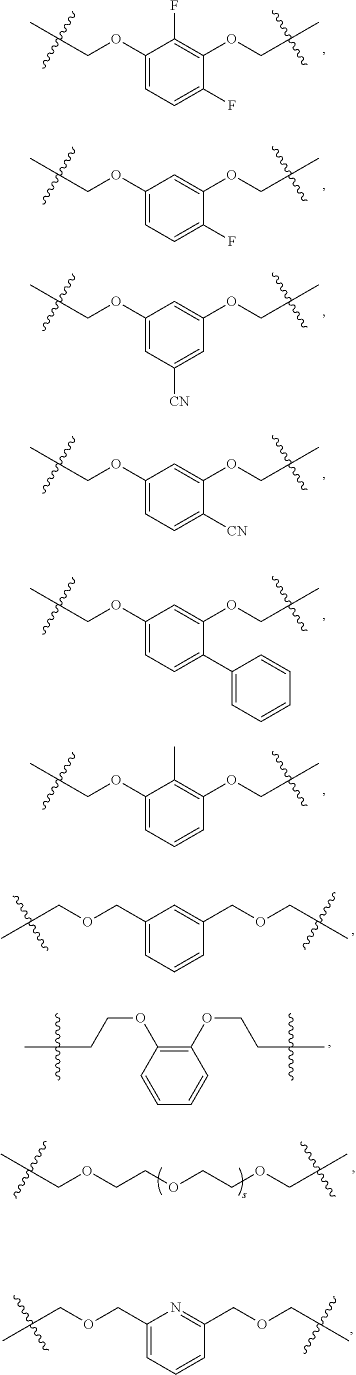 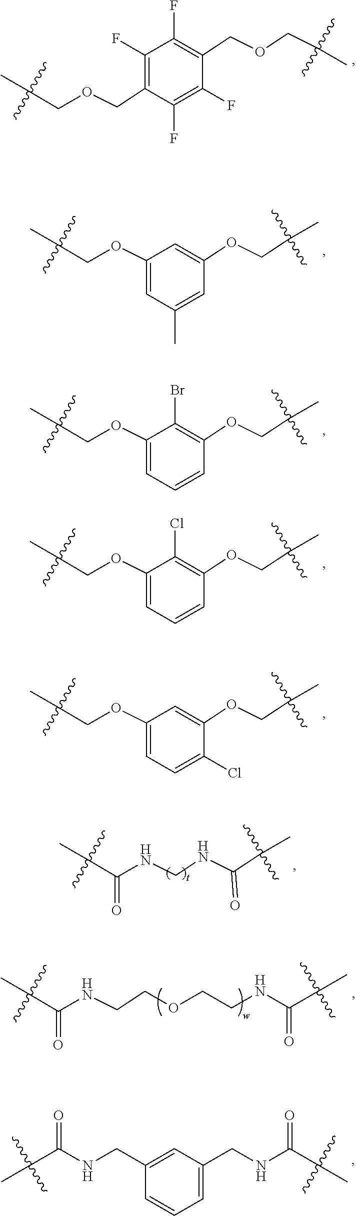 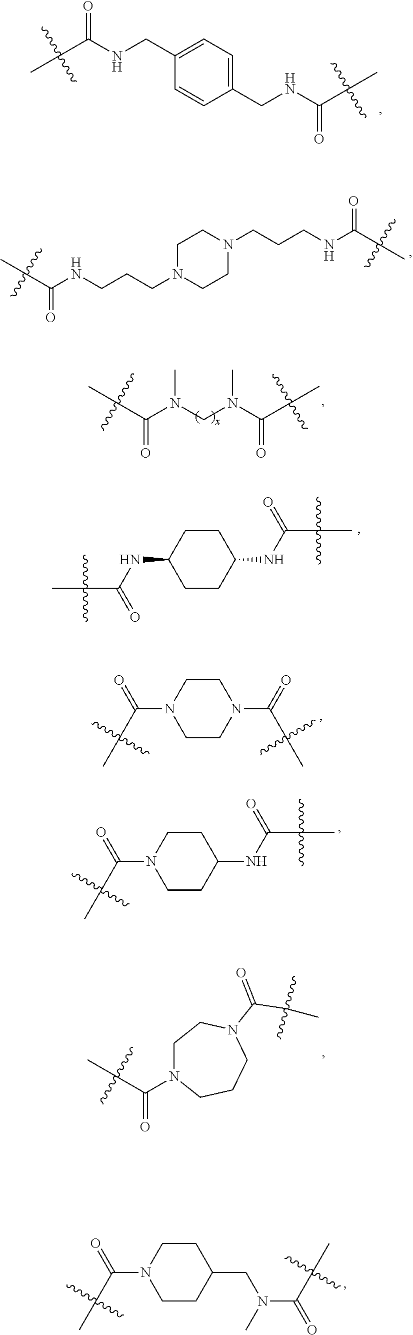 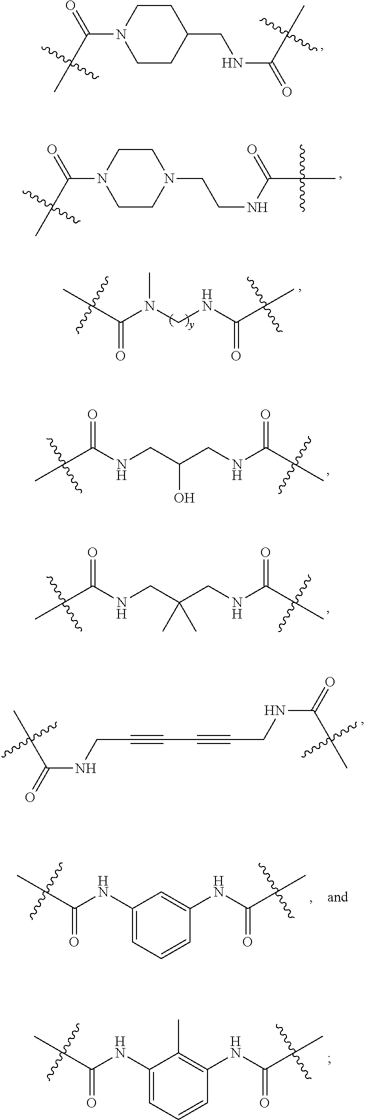
wherein q is an integer from 2 to 8, s in an integer from 1 to 5, t is an integer from 2 to 8, w is an integer from 1 to 5, x is an integer from 2 to 8, and y is an integer from 2 to 8.

12. The compound or pharmaceutically acceptable salt thereof according to claim 1, wherein the compound is

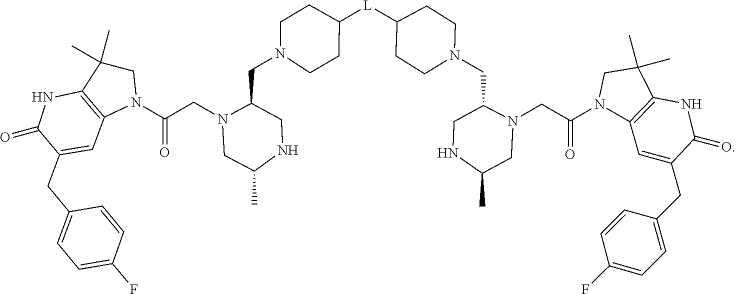

13. The compound or pharmaceutically acceptable salt thereof according to claim 1, wherein L is selected from the group consisting of 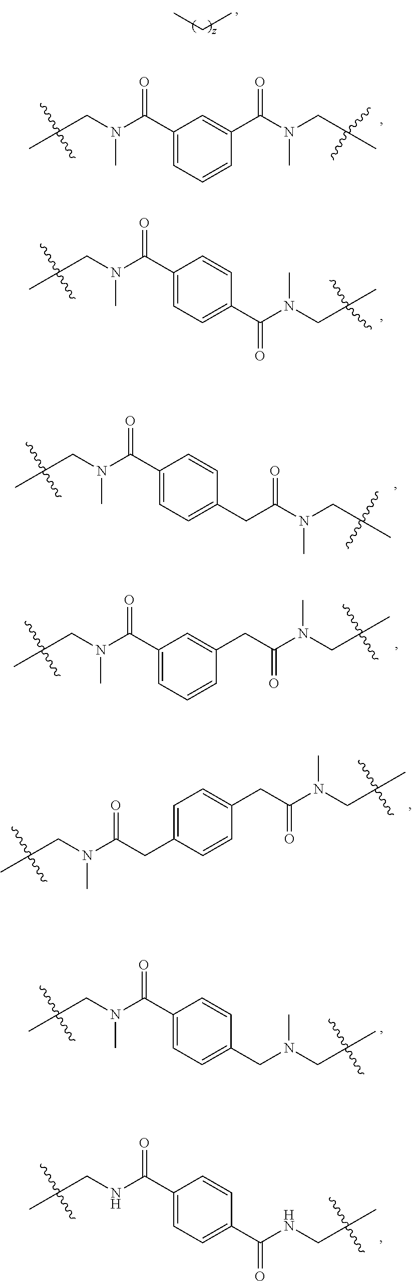 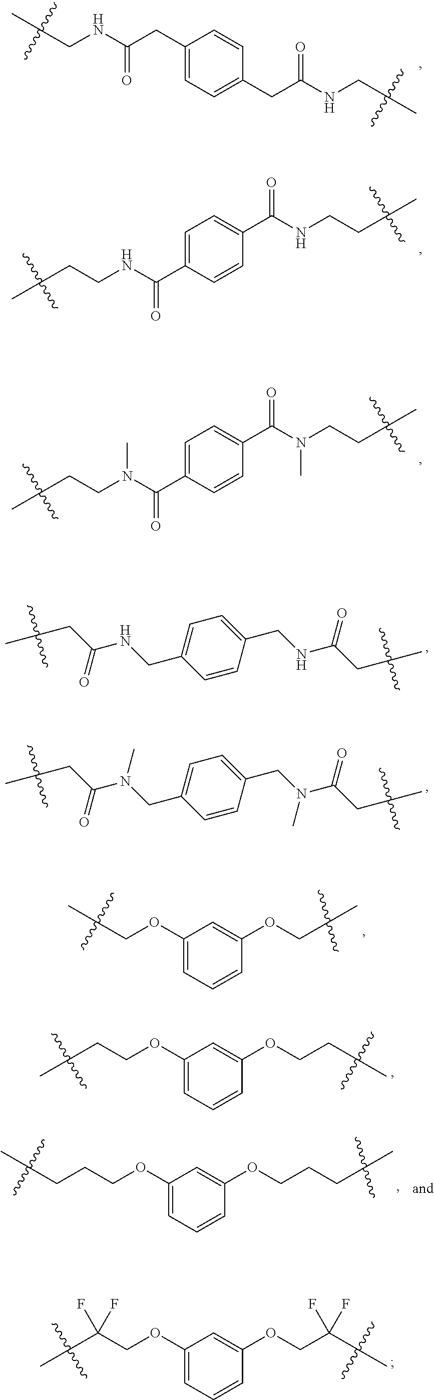
wherein z is an integer from 2 to 8.

14. A compound selected from the group consisting of
1,6-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)hexane-1,6-dione;
1,8-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)octane-1,8-dione;
1,10-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)decane-1,10-dione;
1,14-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)tetradecane-1,14-dione;
1,1′-(2,2′-((3R,3′R,6R,6′R)-(((2R,2′R)-(2,2′-(ethane-1,2-diylbis(oxy))bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(2,2′-((oxybis(ethane-2,1-diyl))bis(oxy))bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(2,2′-(1,2-phenylene)bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(2,2′-(1,2-phenylenebis(oxy))bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-isophthaloylbis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(2,2′-(1,3-phenylene)bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(2,2′-(1,3-phenylenebis(oxy))bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(2,2′-(1,4-phenylene)bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(2,2′-(1,4-phenylenebis(oxy))bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-((2E,2′E)-3,3′-(1,4-phenylene)bis(acryloyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(2,2′-(methylazanediyl)bis(acetyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(pyrazine-2,5-dicarbonyl)bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)—([2,2′-bipyridine]-3,3′-dicarbonyl)bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(naphthalene-2,6-dicarbonyl)bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-terephthaloylbis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(4,4′-((E)-ethene-1,2-diyl)bis(benzoyl))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,4-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)butane-1,4-dione;
1,5-bis((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)pentane-1,5-dione;
1,1′-(2,2′-((3R,3′R,6R,6′R)-(((2R,2′R)—([2,2′-bipyridine]-4,4′-dicarbonyl)bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-octane-1,8-diylbis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-decane-1,10-diylbis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-dodecane-1,12-diylbis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-(1,4-phenylenebis(methylene))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-((perfluoro-1,4-phenylene)bis(methylene))bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)—([1,1′-biphenyl]-4,4′-disulfonyl)bis(2-methylpiperazine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
2,2′-((3R,3′R,6R,6′R)-(((2R,2′R)-(oxybis(4,1-phenylenesulfonyl))bis(2-methylpiperazine-4,1-diyl))bis(methylene))bis(3-methylpiperazine-6,1-diyl))bis(1-(6-(4-fluorobenzyl)-3,3-dimethyl-5-oxo-2,3,4,5-tetrahydro-1H-pyrrolo[3,2-b]pyridin-1-yl)ethan-1-one);
1,14-bis((R)-4-(((2R,5R)-1-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)tetradecane-1,14-dione;
1,6-bis((R)-4-(((2R,5R)-1-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)hexane-1,6-dione;
1,8-bis((R)-4-(((2R,5R)-1-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)octane-1,8-dione;
1,10-bis((R)-4-(((2R,5R)-1-(2-(6-(4-fluorobenzyl)-3,3,4-trimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)decane-1,10-dione;
2,2′-((3R,3′R,6R,6′R)-(((2R,2′R)-isophthaloylbis(2-methylpiperazine-4,1-diyl))bis(methylene))bis(3-methylpiperazine-6,1-diyl))bis(1-(6-(4-fluorobenzyl)-3,3-dimethyl-2,3-dihydro-1H-pyrrolo[3,2-b]pyridin-1-yl)ethan-1-one);
2,2′-((3R,3′R,6R,6′R)-(((2R,2′R)-(1,3-phenylenedisulfonyl)bis(2-methylpiperazine-4,1-diyl))bis(methylene))bis(3-methylpiperazine-6,1-diyl))bis(1-(6-(4-fluorobenzyl)-3,3-dimethyl-2,3-dihydro-1H-pyrrolo[3,2-b]pyridin-1-yl)ethan-1-one);
N1,N6-bis((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)adipamide;
N1,N8-bis((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)octanediamide;
N1,N10-bis((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)decanediamide;
N1,N14-bis((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)tetradecanediamide;
2,2′-(1,3-phenylene)bis(N-((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)acetamide);
2,2′-(1,4-phenylene)bis(N-((S)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)acetamide);
N1,N6-bis((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)adipamide;
N1,N8-bis((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)octanediamide;
N1,N10-bis((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)decanediamide;
N1,N14-bis((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)tetradecanediamide;
2,2′-(1,3-phenylene)bis(N-((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)acetamide);
2,2′-(1,4-phenylene)bis(N-((R)-1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-3-yl)acetamide);
1,1′-(2,2′-((3R,3′R,6R,6′R)-(((2S,2'S)-(((1,2-phenylenebis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)—(([1,1′-biphenyl]-4,4′-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)—(([1,1′-biphenyl]-3,3′-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)—(([1,1′-biphenyl]-3,4′-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((methylenebis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((carbonylbis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((oxybis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((propane-2,2-diylbis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((sulfonylbis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((ethane-1,2-diylbis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,2-bis(4-(((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholin-2-yl)methoxy)phenyl)ethane-1,2-dione;
1,1′-(2,2′-((3R,3′R,6R,6′R)-(((2S,2'S)-((((1,3-phenylenebis(oxy))bis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((((1,3-phenylenebis(propane-2,2-diyl))bis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((((1,4-phenylenebis(propane-2,2-diyl))bis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((hexa-2,4-diyne-1,6-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((hexane-1,6-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((1,4-phenylenebis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((methylenebis(2,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)—(([1,1′-biphenyl]-2,2′-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((oxybis(2,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((1,4-phenylenebis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((1,2-phenylenebis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((2-fluoro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((5-fluoro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((2,4-difluoro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one)
1,1′-(2,2′-((3R,3′R,6R,6′R)-(((2S,2'S)-(((4-fluoro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
3,5-bis(((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholin-2-yl)methoxy)benzonitrile;
2,4-bis(((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholin-2-yl)methoxy)benzonitrile;
1,1′-(2,2′-((3R,3′R,6R,6′R)-(((2S,2'S)—(([1,1′-biphenyl]-2,4-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((2-methyl-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((1,3-phenylenebis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((butane-1,4-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((pentane-1,5-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((oxybis(ethane-2,1-diyl))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((pyridine-2,6-diylbis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((((perfluoro-1,4-phenylene)bis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((5-methyl-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((2-bromo-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((2-chloro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-(((4-chloro-1,3-phenylene)bis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
(2S,2'S)-N,N′-(hexane-1,6-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-((1 S,4S)-cyclohexane-1,4-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(hexa-2,4-diyne-1,6-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
1,1′-(2,2′-((3R,3′R,6R,6′R)-((propane-1,3-diylbis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
N1,N3-bis((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)-N1,N3-dimethylisophthalamide;
N1,N4-bis((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)-N1,N4-dimethylterephthalamide;
N-((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)-4-(2-(((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)(methyl)amino)-2-oxoethyl)-N-methylbenzamide;
N-((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)-3-(2-(((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)(methyl)amino)-2-oxoethyl)-N-methylbenzamide;
2,2′-(1,4-phenylene)bis(N-((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)-N-methylacetamide);
1,1′-(2,2′-((3R,3′R,6R,6′R)-(((((1,4-phenylenebis(methylene))bis(methylazanediyl))bis(methylene))bis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
N1,N4-bis((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)terephthalamide;
2,2′-(1,4-phenylene)bis(N-((1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)methyl)acetamide);
N1,N4-bis(2-(1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)ethyl)terephthalamide;
N1,N4-bis(2-(1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)ethyl)-N1,N4-dimethylterephthalamide;
N,N′-(1,4-phenylenebis(methylene))bis(2-(1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)acetamide);
N,N′-(1,4-phenylenebis(methylene))bis(2-(1-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)piperidin-4-yl)-N-methylacetamide);
1,1′-(2,2′-((3R,3′R,6R,6′R)-((((1,3-phenylenebis(oxy))bis(methylene))bis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-((((1,3-phenylenebis(oxy))bis(ethane-2,1-diyl))bis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-((((1,3-phenylenebis(oxy))bis(propane-3,1-diyl))bis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-((((1,3-phenylenebis(oxy))bis(1,1-difluoroethane-2,1-diyl))bis(piperidine-4,1-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
(2R,2′R)-N,N′-(1,4-phenylenebis(methylene))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
1,1′-(2,2′-((3R,3′R,6R,6′R)-(((2R,2′R)-((1,3-phenylenebis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2R,2′R)-((butane-1,4-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(acetyl))bis(6-(4-fluorobenzyl)-3,3,4-trimethyl-1,2,3,4-tetrahydro-5H-pyrrolo[3,2-b]pyridin-5-one);
1,1′-(2,2′-((3R,3′R,6R,6′R)-(((2S,2'S)-(((methylenebis(4,1-phenylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3,4-trimethyl-1,2,3,4-tetrahydro-5H-pyrrolo[3,2-b]pyridin-5-one);
1,1′-(2,2′-((3R,3′R,6R,6′R)-(((2S,2'S)-((hexa-2,4-diyne-1,6-diylbis(oxy))bis(methylene))bis(morpholine-2,4-diyl))bis(methylene))bis(3-methylpiperazine-6,1-diyl))bis(acetyl))bis(6-(4-fluorobenzyl)-3,3,4-trimethyl-1,2,3,4-tetrahydro-5H-pyrrolo[3,2-b]pyridin-5-one);
1,1′-(2,2′-((3R,3′R,6R,6′R)-(((2S,2'S)-(((1,4-phenylenebis(methylene))bis(oxy))bis(methylene))bis(morpholine-2,4-diyl))bis(methylene))bis(3-methylpiperazine-6,1-diyl))bis(acetyl))bis(6-(4-fluorobenzyl)-3,3,4-trimethyl-1,2,3,4-tetrahydro-5H-pyrrolo[3,2-b]pyridin-5-one);
1,1′-(2,2′-((3R,3′R,6R,6′R)-(((2S,2'S)-(piperazine-1,4-dicarbonyl)bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
(2S,2'S)-N,N′-(octane-1,8-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(decane-1,10-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(((oxybis(ethane-2,1-diyl))bis(oxy))bis(ethane-2,1-diyl))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,3-phenylenebis(methylene))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(piperazine-1,4-diylbis(propane-3,1-diyl))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(hexane-1,6-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-methylmorpholine-2-carboxamide);
(2S,2'S)-N,N′-(propane-1,3-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(butane-1,4-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(butane-1,4-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-methylmorpholine-2-carboxamide);
(2S,2'S)-N,N′-(ethane-1,2-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-(1-((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carbonyl)piperidin-4-yl)morpholine-2-carboxamide;
(2S,2'S)-N,N′-(propane-1,3-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-methylmorpholine-2-carboxamide);
1,1′-(2,2′-((3R,3′R,6R,6′R)-(((2S,2'S)-(1,4-diazepane-1,4-dicarbonyl)bis(morpholine-2,4-diyl))bis(methylene))bis(3-methylpiperazine-6,1-diyl))bis(acetyl))bis(6-(4-fluorobenzyl)-3,3-dimethyl-1,2,3,4-tetrahydro-5H-pyrrolo[3,2-b]pyridin-5-one);
(S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-((1-((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carbonyl)piperidin-4-yl)methyl)-N-methylmorpholine-2-carboxamide;
(S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-((1-((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carbonyl)piperidin-4-yl)methyl)morpholine-2-carboxamide;
(S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-(2-(4-((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carbonyl)piperazin-1-yl)ethyl)morpholine-2-carboxamide;
(S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-(3-((S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamido)propyl)-N-methylmorpholine-2-carboxamide;
(2S,2'S)-N,N′-(pentane-1,5-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(2-hydroxypropane-1,3-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(oxybis(ethane-2,1-diyl))bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(2,2-dimethylpropane-1,3-diyl)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,3-phenylene)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(2-methyl-1,3-phenylene)bis(4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
1-[6-(4-fluorophenoxy)-3,3-dimethyl-2H-pyrrolo[3,2-b]pyridin-1-yl]-2-[(2R,5R)-2-[[(2S)-2-[[3-[[(2S)-4-[[(2R,5R)-1-[2-[6-(4-fluorophenoxy)-3,3-dimethyl-2H-pyrrolo[3,2-b]pyridin-1-yl]-2-oxo-ethyl]-5-methyl-piperazin-2-yl]methyl]morpholin-2-yl]methoxy]phenoxy]methyl]morpholin-4-yl]methyl]-5-methyl-piperazin-1-yl]ethenone;
-[6-(4-fluoroanilino)-3,3-dimethyl-2H-pyrrolo[3,2-b]pyridin-1-yl]-2-[(2R,5R)-2-[[(2S)-2-[[3-[[(2S)-4-[[(2R,5R)-1-[2-[6-(4-fluoroanilino)-3,3-dimethyl-2H-pyrrolo[3,2-b]pyridin-1-yl]-2-oxo-ethyl]-5-methyl-piperazin-2-yl]methyl]morpholin-2-yl]methoxy]phenoxy]methyl]morpholin-4-yl]methyl]-5-methyl-piperazin-1-yl]ethenone;
2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(6-((4-fluorophenyl)(methyl)amino)-3,3-dimethyl-2,3-dihydro-1H-pyrrolo[3,2-b]pyridin-1-yl)ethan-1-one);
2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(6-(4-fluorobenzyl)-3,3-dimethyl-2,3-dihydro-1H-pyrrolo[3,2-b]pyridin-1-yl)ethan-1-one);
2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(3,3-dimethyl-6-(pyridin-3-ylmethyl)-2,3-dihydro-1H-pyrrolo[3,2-b]pyridin-1-yl)ethan-1-one);
2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(6-(4-chlorobenzyl)-3,3-dimethyl-2,3-dihydro-1H-pyrrolo[3,2-b]pyridin-1-yl)ethan-1-one);
2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(3,3-dimethyl-6-(4-methylbenzyl)-2,3-dihydro-1H-pyrrolo[3,2-b]pyridin-1-yl)ethan-1-one);
2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(3,3-dimethyl-6-(4-(trifluoromethyl)benzyl)-2,3-dihydro-1H-pyrrolo[3,2-b]pyridin-1-yl)ethan-1-one);
2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(6-benzyl-3,3-dimethyl-2,3-dihydro-1H-pyrrolo[3,2-b]pyridin-1-yl)ethan-1-one);
2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(1-(6-(4-fluoro-2-methoxybenzyl)-3,3-dimethyl-2,3-dihydro-1H-pyrrolo[3,2-b]pyridin-1-yl)ethan-1-one);
dimethyl 6,6′-(((2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(acetyl))bis(3,3-dimethyl-2,3-dihydro-1H-pyrrolo[3,2-b]pyridine-1,6-diyl))bis(methylene))bis(3-fluorobenzoate);
1-[6-[(4-bromophenyl)methyl]-3,3-dimethyl-2H-pyrrolo[3,2-b]pyridin-1-yl]-2-[(2R,5R)-2-[[(2S)-2-[[3-[[(2S)-4-[[(2R,5R)-1-[2-[6-[(4-bromophenyl)methyl]-3,3-dimethyl-2H-pyrrolo[3,2-b]pyridin-1-yl]-2-oxo-ethyl]-5-methyl-piperazin-2-yl]methyl]morpholin-2-yl]methoxy]phenoxy]methyl]morpholin-4-yl]methyl]-5-methyl-piperazin-1-yl]ethenone;
6-(4-fluorobenzyl)-1-(2-((2R,5R)-2-(((R)-4-(2-(1-(3-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-3-oxopropyl)-1H-1,2,3-triazol-4-yl)ethyl)-2-methylpiperazin-1-yl)methyl)-5-methylpiperazin-1-yl)acetyl)-3,3-dimethyl-1,2,3,4-tetrahydro-5H-pyrrolo[3,2-b]pyridin-5-one;
6-(4-fluorobenzyl)-1-(2-((2R,5R)-2-(((R)-4-((1-(2-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-2-oxoethyl)-1H-1,2,3-triazol-4-yl)methyl)-2-methylpiperazin-1-yl)methyl)-5-methylpiperazin-1-yl)acetyl)-3,3-dimethyl-1,2,3,4-tetrahydro-5H-pyrrolo[3,2-b]pyridin-5-one;
6-(4-fluorobenzyl)-1-(2-((2R,5R)-2-(((R)-4-((1-(3-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-3-oxopropyl)-1H-1,2,3-triazol-4-yl)methyl)-2-methylpiperazin-1-yl)methyl)-5-methylpiperazin-1-yl)acetyl)-3,3-dimethyl-1,2,3,4-tetrahydro-5H-pyrrolo[3,2-b]pyridin-5-one;
6-(4-fluorobenzyl)-1-(2-((2R,5R)-2-(((R)-4-(2-(1-(4-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-4-oxobutyl)-1H-1,2,3-triazol-4-yl)ethyl)-2-methylpiperazin-1-yl)methyl)-5-methylpiperazin-1-yl)acetyl)-3,3-dimethyl-1,2,3,4-tetrahydro-5H-pyrrolo[3,2-b]pyridin-5-one;
6-(4-fluorobenzyl)-1-(2-((2R,5R)-2-(((R)-4-(2-(1-(5-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-5-oxopentyl)-1H-1,2,3-triazol-4-yl)ethyl)-2-methylpiperazin-1-yl)methyl)-5-methylpiperazin-1-yl)acetyl)-3,3-dimethyl-1,2,3,4-tetrahydro-5H-pyrrolo[3,2-b]pyridin-5-one;
(S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-(2-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-2-oxoethyl)morpholine-2-carboxamide;
(S)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-N-(4-((R)-4-(((2R,5R)-1-(2-(6-(4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)-3-methylpiperazin-1-yl)-4-oxobutyl)morpholine-2-carboxamide;
(2S,2'S)-N,N′-(1,4-phenylenebis(methylene))bis(4-(((2R,5R)-1-(2-(6-(4-chlorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-bromobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(3,3-dimethyl-6-(4-methylbenzyl)-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-chloro-2-hydroxybenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(2,4-dichlorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-(difluoromethyl)benzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(3,3-dimethyl-5-oxo-6-(2,4,6-trifluorobenzyl)-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-chloro-2-methylbenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-chloro-2-methoxybenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-chloro-2-(trifluoromethyl)benzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-chloro-2-cyanobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-chloro-2-(dimethylcarbamoyl)benzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(2-isopropylbenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(3,3-dimethyl-5-oxo-6-(2,3,4,5-tetrafluorobenzyl)-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(3,3-dimethyl-5-oxo-6-(2,4,5-trifluorobenzyl)-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(3,3-dimethyl-5-oxo-6-((perfluorophenyl)methyl)-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(2,4-difluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(3,3-dimethyl-5-oxo-6-(2,3,4-trifluorobenzyl)-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(2-cyano-4-fluorobenzyl)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(3,3-dimethyl-5-oxo-6-(2,4,6-trichlorobenzyl)-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-fluorophenoxy)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-chlorophenoxy)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-(4-bromophenoxy)-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(3,3-dimethyl-5-oxo-6-(p-tolyloxy)-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(3,3-dimethyl-5-oxo-6-phenoxy-2,3,4,5-tetrahydro-1H-pyrrolo[3,2-b]pyridin-1-yl)-2-oxoethyl)-5-methylpiperazin-2-yl)methyl)morpholine-2-carboxamide);
(2S,2'S)-N,N′-(1,4-phenylenebis(methylene))bis(4-(((2R,5R)-1-(2-(6-benzyl-3,3-dimethyl-5-oxo-2,3,4,5-tetrahydro-1H-pyrrolo[3,2-b]pyridin-1-yl)-2-oxoethyl)-5-methylpiperazin-2-yl)methyl)morpholine-2-carboxamide);
1,1′-(2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(acetyl))bis(6-(2,4-dichlorobenzyl)-3,3-dimethyl-1,2,3,4-tetrahydro-5H-pyrrolo[3,2-b]pyridin-5-one);
1,1′-(2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(acetyl))bis(3,3-dimethyl-6-(2,4,6-trifluorobenzyl)-1,2,3,4-tetrahydro-5H-pyrrolo[3,2-b]pyridin-5-one);
6,6′-(((2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6, 1-diyl))bis(acetyl))bis(3,3-dimethyl-5-oxo-2,3,4,5-tetrahydro-1H-pyrrolo[3,2-b]pyridine-1,6-diyl))bis(methylene))bis(3-chlorobenzonitrile); and
1,1′-(2,2′-((3R,3′R,6R,6′R)-(((2S,2'S)-((1,3-phenylenebis(oxy))bis(methylene))bis(morpholine-2,4-diyl))bis(methylene))bis(3-methylpiperazine-6,1-diyl))bis(acetyl))bis(6-(2,4-difluorobenzyl)-3,3-dimethyl-1,2,3,4-tetrahydro-5H-pyrrolo[3,2-b]pyridin-5-one);
or a pharmaceutically acceptable salt thereof.

15. A pharmaceutical composition comprising the compound or pharmaceutically acceptable salt thereof according to claim 1, and a pharmaceutically acceptable excipient.

16. A method of treating an HIV infection in a human comprising administering to the human a therapeutically effective amount of the compound or pharmaceutically acceptable salt thereof according to claim 1.

17. A method of treating an HIV infection in a human comprising administering to the human a therapeutically effective amount of pharmaceutical composition according to claim 15.

18. (canceled)

19. A method of treating cancer and pre-cancerous syndromes, in a human in need thereof, which comprises administering to the human a therapeutically effective amount of the compound or pharmaceutically acceptable salt thereof according to claim 1.

20. A method of depleting latent HIV infected cells in a human comprising administering to the human a therapeutically effective amount of the compound or pharmaceutically acceptable salt thereof according to claim 1.

21. A combination comprising the compound or pharmaceutically acceptable salt thereof according to claim 1, and one or more pharmaceutical agents active against HIV.

22. The combination according to claim 21, wherein the one or more pharmaceutical agents active against HIV is selected from the group consisting of nucleotide reverse transcriptase inhibitors, non-nucleotide reverse transcriptase inhibitors, protease inhibitors, entry inhibitors, attachment and fusion inhibitors, integrase inhibitors, maturation inhibitors, CXCR4 and/or CCR5 inhibitors, histone deacetylase inhibitors, histone crotonyl transferase inhibitors, protein kinase C agonists, proteasome inhibitors, TLR7 agonists, bromodomain inhibitors, and antibodies for clearance therapy.

23. A method of treating an HIV infection in a human comprising administering to the human a therapeutically effective amount of the combination according to claim 21.

24. A method of depleting latent HIV infected cells in a human comprising administering to the human a therapeutically effective amount of the combination according to claim 21.


SEQUENCE LISTING (4 sequences)

SEQ ID NO: 1 - DNA, 22 bp, synthetic construct
  misc_feature 1..22: note = synthetic primer
  source 1..22: mol_type = other DNA
atcaagcagctatgcaaatgtt

SEQ ID NO: 2 - DNA, 30 bp, synthetic construct
  misc_feature 1..30: note = synthetic primer
  source 1..30: mol_type = other DNA
ctgaagggtactagtagttcctgctatgtc

SEQ ID NO: 3 - DNA, 29 bp, synthetic construct
  misc_feature 1..29: note = synthetic probe
  source 1..29: mol_type = other DNA
accatcaatgaggaagctgcagaatggga

SEQ ID NO: 4 - DNA, 152 bp, synthetic construct
  misc_feature 1..152: note = synthetic standard control
  source 1..152: mol_type = other DNA
atcaagcagccatgcaaatgttaaaagagaccatcaatgaggaagctgcagaatgggatagattgcatccagtgcatgcagggcctattgcaccaggccagatgagagaaccaaggggaagtgacatagcaggaactactagtacccttcag